(19) United States
(12) Patent Application Publication
(10) Pub. No.: US 2024/0057473 A1
(43) Pub. Date: Feb. 15, 2024

(54) LIGHT EMITTING ELEMENT AND AMINE COMPOUND FOR THE SAME

(71) Applicant: Samsung Display Co., Ltd., Yongin-si (KR)

(72) Inventors: HANKYU PAK, Yongin-si (KR); MINJI KIM, Yongin-si (KR); BYEONGWOOK YOO, Yongin-si (KR); Jeongmin LEE, Yongin-si (KR); SOHEE JO, Yongin-si (KR)

(21) Appl. No.: 18/349,010

(22) Filed: Jul. 7, 2023

(30) Foreign Application Priority Data
Jul. 19, 2022 (KR) 10-2022-0089180

(51) Int. Cl. H10K 85/60; C07C 211/61; C09K 11/06

(52) U.S. Cl. CPC H10K 85/633; C07C 211/61; C09K 11/06; H10K 50/156

(57) ABSTRACT

A light emitting element of one or more embodiments includes a first electrode, a second electrode provided on the first electrode, and at least one functional layer provided between the first electrode and the second electrode, wherein the functional layer includes an amine compound including a spiro-bonded linker of two hydrocarbon rings and two amine groups connected with the linker. The light emitting element may have improved emission efficiency and element life.

### CROSS-REFERENCE TO RELATED APPLICATIONS

This application claims priority to and the benefit of Korean Patent Application No. 10-2022-0089180, filed on Jul. 19, 2022, in the Korean Intellectual Property Office, the entire content of which is incorporated herein by reference.

### BACKGROUND

### 1. Field

One or more aspects of embodiments of the present disclosure herein are directed to an amine compound and a light emitting element including the same, and more particularly, to a light emitting element including a novel amine compound in a hole transport region.

### 2. Description of Related Art

In the recent years, the development of an organic electroluminescence display device as an image display device is being actively conducted. The organic electroluminescence display device, etc. is a display device including a self-luminescent light emitting element in which holes and electrons injected from a first electrode and a second electrode recombine in an emission layer so that a light emitting material in the emission layer emits light to achieve display of images.

In the application of a light emitting element to a display device, the decrease of a driving voltage, the improvement of emission efficiency, and/or the increase of lifetime is (are) required (or desired), and development of materials for a light emitting element, capable of suitably achieving one or more of these characteristics is being consistently required (or desired).

In addition, in an effort to accomplish a light emitting element with high efficiency, the development of the materials for a hole transport region to suppress or reduce the diffusion of the exciton energy of an emission layer, etc., is being conducted.

### SUMMARY

One or more aspects of embodiments of the present disclosure are directed toward a light emitting element having reduced driving voltage, excellent (or improved) emission efficiency, and/or long-life characteristic(s).

One or more aspects of embodiments of the present disclosure are directed toward an amine compound which is a material for a light emitting element having reduced driving voltage, high efficiency, and/or long-life characteristic(s).

One or more embodiments provide a light emitting element including a first electrode, a second electrode provided on the first electrode, and at least one functional layer provided between the first electrode and the second electrode, and including an amine compound represented by Formula 1.

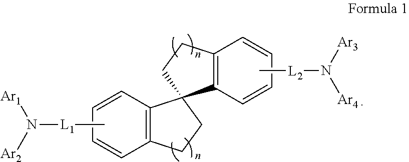

In Formula 1, Ar₁ to Ar₄ may be each independently a substituted or unsubstituted hydrocarbon ring of 6 to 30 ring-forming carbon atoms, or a substituted or unsubstituted heteroaryl group of 2 to 30 ring-forming carbon atoms. L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. “n” may be an integer of 1 to 3.

In one or more embodiments, the at least one functional layer may include an emission layer, a hole transport region provided between the first electrode and the emission layer, and an electron transport region provided between the emission layer and the second electrode, and the hole transport region may include the amine compound.

In one or more embodiments, the hole transport region may include a hole injection layer provided on the first electrode, and a hole transport layer provided on the hole injection layer, and at least one selected from the hole injection layer and the hole transport layer may include the amine compound.

In one or more embodiments, the hole transport region may include a first hole transport layer provided adjacent to the first electrode, a second hole transport layer provided on the first hole transport layer, and a third hole transport layer provided on the second hole transport layer.

In one or more embodiments, the first hole transport layer and the third hole transport layer may include the amine compound, and the second hole transport layer may include a compound represented by Formula H-1.

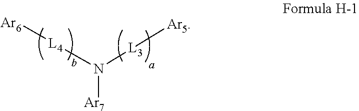

In Formula H-1, Ar₅ and Ar₆ may be each independently a substituted or unsubstituted aryl group of 6 to 30 ring-forming carbon atoms, or a substituted or unsubstituted heteroaryl group of 2 to 30 ring-forming carbon atoms. Ar₇ may be a substituted or unsubstituted aryl group of 6 to 30 ring-forming carbon atoms. L₃ and L₄ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10.

In one or more embodiments, the hole transport region may include a hole injection layer provided on the first electrode, a hole transport layer provided on the hole injection layer, and an electron blocking layer provided on the hole transport layer, and at least one selected from the hole injection layer, the hole transport layer, and the electron blocking layer may include the amine compound.

In one or more embodiments, the amine compound represented by Formula 1 may be represented by Formula 2-1 or Formula 2-2.

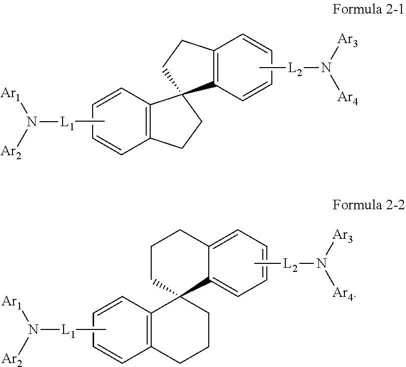

In Formula 2-1 and Formula 2-2, Ar₁ to Ar₄, L₁ and L₂ are the same as defined in Formula 1.

In one or more embodiments, the amine compound represented by Formula 1 may be represented by Formula 3-1 or Formula 3-2.

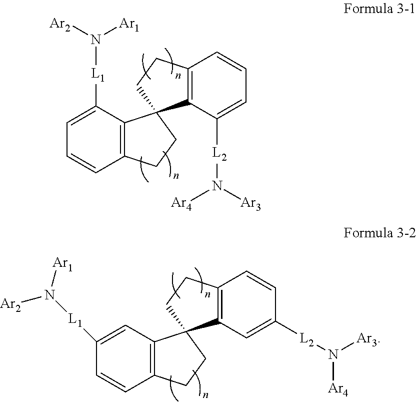

In Formula 3-1 and Formula 3-2, Ar₁ to Ar₄, L₁, L₂, and “n” are the same as defined in Formula 1.

In one or more embodiments, the amine compound represented by Formula 1 may be represented by Formula 4-1.

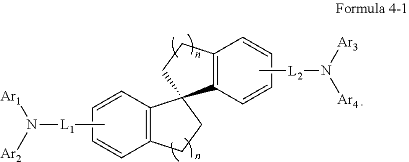

In Formula 4-1, Ar₁ to Ar₄, and “n” are the same as defined in Formula 1.

In one or more embodiments, Ar₁ to Ar₄ in the amine compound represented by Formula 1 may be each independently represented by Formula 1-1a or Formula 1-1b.

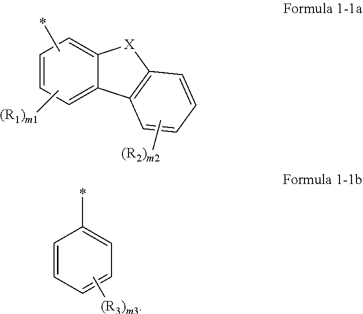

In Formula 1-1a, X may be O, S, CRₓR_{y}, or NR_{z}, and Rₓ, R_{y}, and R_{z} may be each independently a hydrogen atom, a deuterium atom, a substituted or unsubstituted alkyl group of 1 to 10 carbon atoms, or a substituted or unsubstituted aryl group of 6 to 15 ring-forming carbon atoms. In Formula 1-1a and Formula 1-1b, R₁ to R₃ may be each independently a hydrogen atom, a deuterium atom, a substituted or unsubstituted alkyl group of 1 to 10 carbon atoms, a substituted or unsubstituted cycloalkyl group of 6 to 15 ring-forming carbon atoms, or a substituted or unsubstituted aryl group of 6 to 15 ring-forming carbon atoms, and/or combined with an adjacent group to form a ring. m1 may be an integer of 0 to 3, m2 may be an integer of 0 to 4, and m3 may be an integer of 0 to 5.

In one or more embodiments, the amine compound represented by Formula 1 may be represented by Formula 5-1 or Formula 5-2.

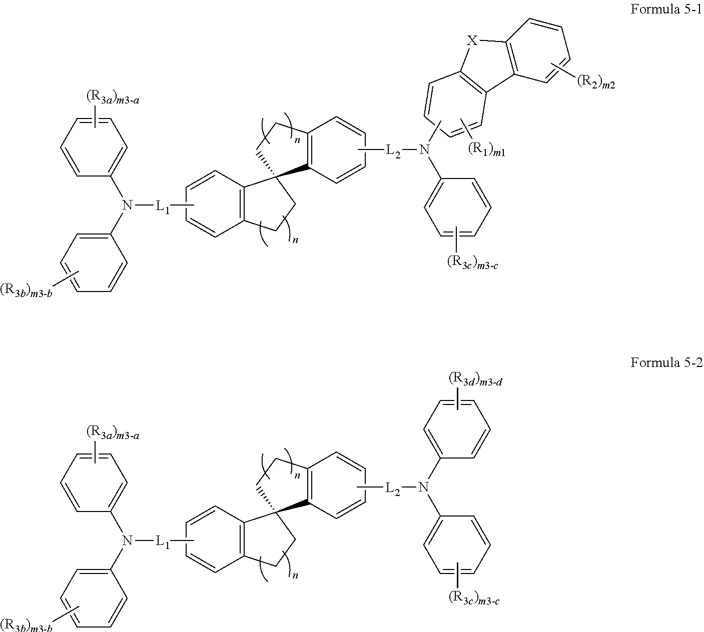

In Formula 5-1 and Formula 5-2, L₁, L₂, and “n” are the same as defined in Formula 1. X, R₁, R₂, m1 and m2 are the same as defined in Formula 1-1a, R₃ₐ, R_{3b}, R_{3c}, and R_{3d} are each independently the same as R₃ defined in Formula 1-1b, and m3-a, m3-b, m3-c, and m3-d are each independently the same as m3 defined in Formula 1-1b.

An amine compound according to one or more embodiments of the present disclosure may be represented by Formula 1 above.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view showing a display device according to one or more embodiments;

FIG. 2 is a partial cross-sectional view taken along line I-I′ in FIG. 1;

FIG. 3 is a cross-sectional view schematically showing a light emitting element of one or more embodiments;

FIG. 4 is a cross-sectional view schematically showing a light emitting element of one or more embodiments;

FIG. 5 is a cross-sectional view schematically showing a light emitting element of one or more embodiments;

FIG. 6 is a cross-sectional view schematically showing a light emitting element of one or more embodiments;

FIG. 7 is a cross-sectional view schematically showing a light emitting element of one or more embodiments;

FIG. 8 is a cross-sectional view showing a display device according to one or more embodiments;

FIG. 9 is a cross-sectional view showing a display device according to one or more embodiments;

FIG. 10 is a cross-sectional view showing a display device according to one or more embodiments; and

FIG. 11 is a cross-sectional view showing a display device according to one or more embodiments.

### DETAILED DESCRIPTION

The present disclosure may have various modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

In the description, when an element (or a region, a layer, a part, etc.) is referred to as being “on”, “connected with” or “combined with” another element, it can be directly connected with/bonded on the other element (e.g., without any third elements therebetween), or one or more intervening third elements may also be provided.

Like reference numerals refer to like elements throughout. In the drawings, the thicknesses, ratios, and dimensions of elements are exaggerated for effective explanation of technical contents.

As used herein, expressions such as “at least one of”, “one of”, and “selected from”, when preceding a list of elements, modify the entire list of elements and do not modify the individual elements of the list. For example, “at least one selected from a, b and c”, “at least one of a, b or c”, and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” may include one or more combinations that may define relevant elements. Further, the use of “may” when describing embodiments of the present disclosure refers to “one or more embodiments of the present disclosure.”

It will be understood that, although the terms first, second, etc. may be used herein to describe various elements, these elements should not be limited by these terms. These terms are only used to distinguish one element from another element. For example, a first element could be termed a second element without departing from the scope of the present invention. Similarly, a second element could be termed a first element. As used herein, the singular forms are intended to include the plural forms as well, unless the context clearly indicates otherwise.

In addition, the terms “below”, “beneath”, “on” and “above” are used for explaining the relation of elements shown in the drawings. The terms are relative concept and are explained based on the direction shown in the drawing.

It will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

Unless otherwise defined, all terms (including technical and scientific terms) used herein have the same meaning as commonly understood by one of ordinary skill in the art to which this present disclosure belongs. In addition, it will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the description, when a layer, a film, a region, a plate, etc. is referred to as being “on” or “above” another part, it can be “directly on” the other part (e.g., without any intervening layers therebetween), or intervening layers may also be present. Similarly, when a layer, a film, a region, a plate, etc. is referred to as being “under” or “below” another part, it can be “directly under” the other part (e.g., without any intervening layers therebetween), or intervening layers may also be present. Also, when an element is referred to as being provided “on” another element, it can be provided under the other element.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic device and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the description, the term “substituted or unsubstituted” corresponds to a group that is unsubstituted or that is substituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, a heterocyclic group, and combinations thereof. In addition, each of the exemplified substituents may itself be substituted or unsubstituted. For example, a biphenyl group may be interpreted as an aryl group or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the description, the term “adjacent group” may refer to a pair of substituent groups where the first substituent is connected to an atom which is directly connected to another atom substituted with the second substituent; a pair of substituent groups connected to the same atom; or a pair of substituent groups where the first substituent is sterically positioned at the nearest position to the second substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, a halogen atom may be a fluorine atom, a chlorine atom, a bromine atom, and/or an iodine atom.

In the description, an alkyl group may be a linear, branched, or cyclic alkyl group. The carbon number of the alkyl group may be 1 to 50, 1 to 30, 1 to 20, 1 to 10, or 1 to 6. Examples of the alkyl group may include methyl group, ethyl group, n-propyl group, isopropyl group, n-butyl group, s-butyl group, t-butyl group, i-butyl group, 2-ethylbutyl group, 3,3-dimethylbutyl group, n-pentyl group, i-pentyl group, neopentyl group, t-pentyl group, cyclopentyl group, 1-methylpentyl group, 3-methylpentyl group, 2-ethylpentyl group, 4-methyl-2-pentyl group, n-hexyl group, 1-methylhexyl group, 2-ethylhexyl group, 2-butylhexyl group, cyclohexyl group, 4-methylcyclohexyl group, 4-t-butylcyclohexyl group, n-heptyl group, 1-methylheptyl group, 2,2-dimethylheptyl group, 2-ethylheptyl group, 2-butylheptyl group, n-octyl group, t-octyl group, 2-ethyloctyl group, 2-butyloctyl group, 2-hexyloctyl group, 3,7-dimethyloctyl group, cyclooctyl group, n-nonyl group, n-decyl group, adamantyl group, 2-ethyldecyl group, 2-butyldecyl group, 2-hexyldecyl group, 2-octyldecyl group, n-undecyl group, n-dodecyl group, 2-ethyldodecyl group, 2-butyldodecyl group, 2-hexyldocecyl group, 2-octyldodecyl group, n-tridecyl group, n-tetradecyl group, n-pentadecyl group, n-hexadecyl group, 2-ethylhexadecyl group, 2-butylhexadecyl group, 2-hexylhexadecyl group, 2-octylhexadecyl group, n-heptadecyl group, n-octadecyl group, n-nonadecyl group, n-eicosyl group, 2-ethyleicosyl group, 2-butyleicosyl group, 2-hexyleicosyl group, 2-octyleicosyl group, n-henicosyl, n-docosyl group, n-tricosyl group, n-tetracosyl group, n-pentacosyl group, n-hexacosyl group, n-heptacosyl group, n-octacosyl group, n-nonacosyl group, n-triacontyl group, etc., without limitation.

In the description, an alkenyl group means a hydrocarbon group including one or more carbon double bonds in the middle and/or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, a 1-butenyl group, a 1-pentenyl group, a 1,3-butadienyl group, a styrenyl group, a styrylvinyl group, etc., without limitation.

In the description, an alkynyl group means a hydrocarbon group including one or more carbon triple bonds in the middle or at the terminal of an alkyl group having a carbon number of 2 or more. The alkynyl group may be a linear chain or a branched chain. The carbon number is not specifically limited, but may be 2 to 30, 2 to 20, or 2 to 10. Particular examples of the alkynyl group may include an ethynyl group, a propynyl group, etc., without limitation.

In the description, a hydrocarbon ring group means an optional functional group or substituent derived from an aliphatic hydrocarbon ring. The hydrocarbon ring group may be a saturated hydrocarbon ring group of 5 to 20 ring-forming carbon atoms.

In the description, an aryl group means an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl group, naphthyl group, fluorenyl group, anthracenyl group, phenanthryl group, biphenyl group, terphenyl group, quaterphenyl group, quinquephenyl group, sexiphenyl group, triphenylenyl group, pyrenyl group, benzofluoranthenyl group, chrysenyl group, etc., without limitation.

In the description, a fluorenyl group may be substituted, and two substituents may be combined with each other to form a spiro structure. Examples of a substituted fluorenyl group are as follows, but one or more embodiments of the present disclosure is not limited thereto.

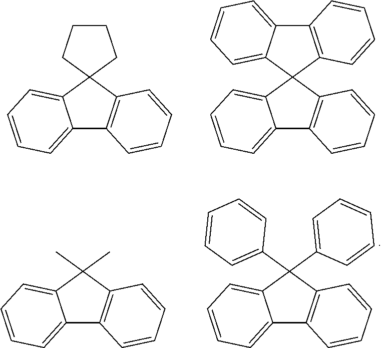

In the description, a heterocyclic group means an optional functional group or substituent derived from a ring including one or more selected from B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may each independently be a monocycle or a polycycle.

In the description, a heterocyclic group may include one or more selected from B, O, N, P, Si and S as heteroatoms. If the heterocyclic group includes two or more heteroatoms, two or more heteroatoms may be the same or different. In the description, the heterocyclic group may be a monocyclic heterocyclic group or polycyclic heterocyclic group and has concept including a heteroaryl group. The carbon number for forming rings of the heterocyclic group may be 2 to 30, 2 to 20, or 2 to 10.

In the description, an aliphatic heterocyclic group may include one or more selected from B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, a heteroaryl group may include one or more selected from B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene group, furan group, pyrrole group, imidazole group, triazole group, pyridine group, bipyridine group, pyrimidine group, triazine group, triazole group, acridyl group, pyridazine group, pyrazinyl group, quinoline group, quinazoline group, quinoxaline group, phenoxazine group, phthalazine group, pyrido pyrimidine group, pyrido pyrazine group, pyrazino pyrazine group, isoquinoline group, indole group, carbazole group, N-arylcarbazole group, N-heteroarylcarbazole group, N-alkylcarbazole group, benzoxazole group, benzoimidazole group, benzothiazole group, benzocarbazole group, benzothiophene group, dibenzothiophene group, thienothiophene group, benzofuran group, phenanthroline group, thiazole group, isooxazole group, oxazole group, oxadiazole group, thiadiazole group, phenothiazine group, dibenzosilole group, dibenzofuran group, etc., without limitation.

In the description, the same explanation as provided for the above-described aryl group may be applied to an arylene group except that the arylene group is a divalent group. The same explanation as provided for the above-described heteroaryl group may be applied to a heteroarylene group except that the heteroarylene group is a divalent group.

In the description, a boron group may mean the above-defined alkyl group or aryl group bonded to a boron atom. The boron group may include an alkyl boron group and an aryl boron group. Examples of the boron group may include a dimethylboron group, a diethylboron group, a t-butylmethylboron group, a diphenylboron group, a phenylboron group, etc., without limitation.

In the description, a silyl group includes an alkyl silyl group and an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, the carbon number of a carbonyl group is not specifically limited, but the carbon number may be 1 to 40, 1 to 30, or 1 to 20. For example, the carbonyl group may have the structures below, but is not limited thereto.

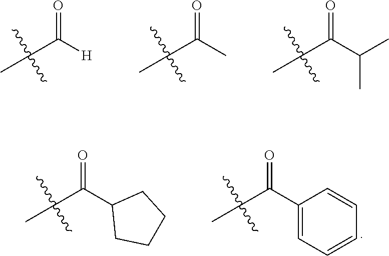

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may mean the above-defined alkyl group or aryl group combined with a sulfur atom. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may mean the above-defined alkyl group or aryl group which is combined with an oxygen atom. The oxy group may include an alkoxy group and an aryl oxy group. The alkoxy group may include a linear, branched and/or cyclic alkyl group chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, one or more embodiments of the present disclosure is not limited thereto.

In the description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., without limitation.

In the description, alkyl groups in an alkyl thio group, alkyl sulfinyl group, alkyl sulfonyl group, an alkoxy group, an alkyl boron group, an alkyl silyl group and an alkyl amine group may be the same as the examples of the above-described alkyl group.

In the description, aryl groups in an aryl oxy group, aryl thio group, aryl sulfinyl group, an aryl sulfonyl group, an aryl boron group, aryl silyl group, and an aryl amine group may be the same as the examples of the above-described aryl group.

In the description, a direct linkage may mean a single bond.

Meanwhile, in the description,

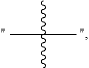

or “-*” means a position to be connected (e.g., a bonding site).

Hereinafter, a light emitting element according to one or more embodiments of the present disclosure and a display device including the same will be explained referring to attached drawings.

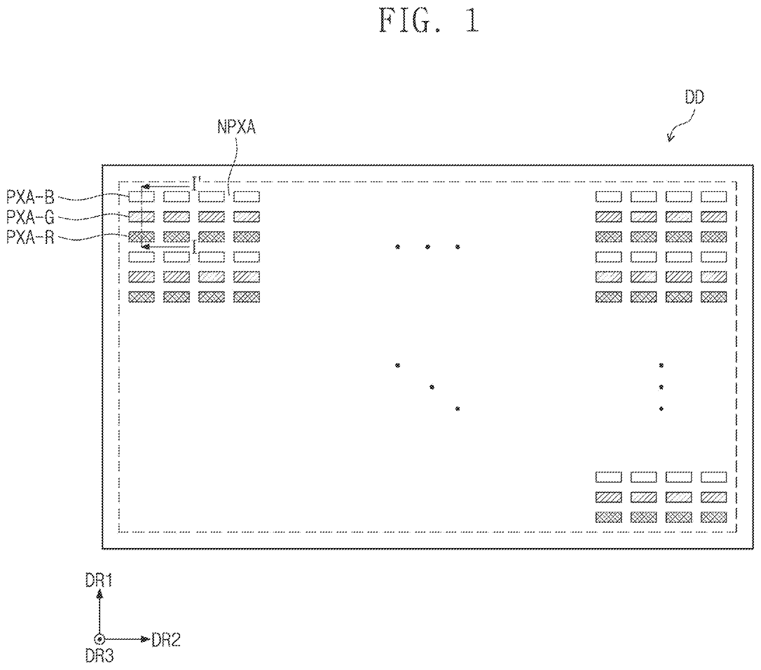

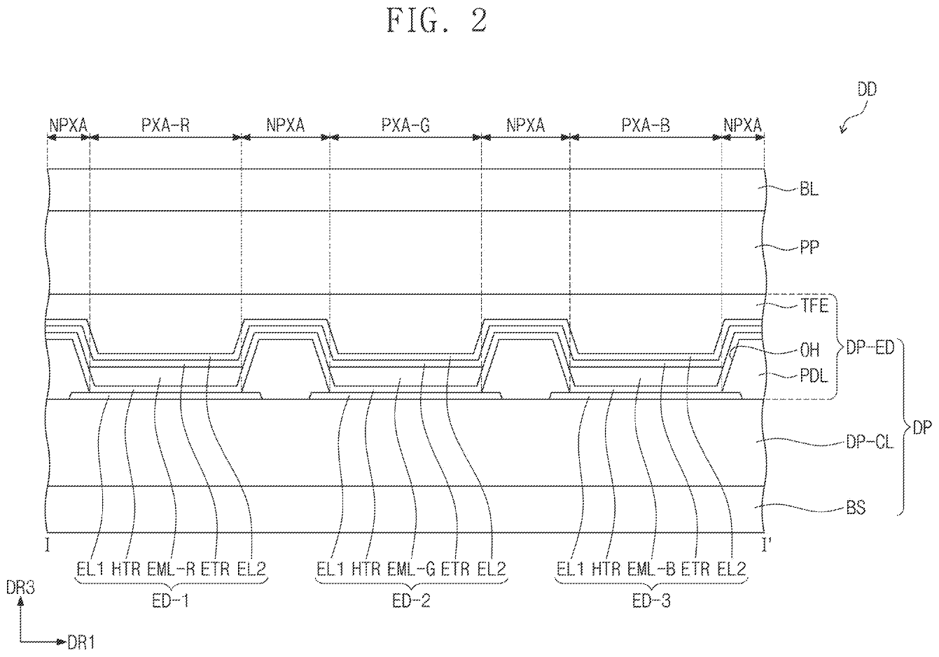

FIG. 1 is a plan view showing a display device DD according to one or more embodiments. FIG. 2 is a cross-sectional view of a display device DD of one or more embodiments. FIG. 2 is a cross-sectional view showing a part corresponding to line I-I′ of FIG. 1.

The display device DD may include a display panel DP and an optical layer PP provided on the display panel DP. The display panel DP may include light emitting elements ED-1, ED-2 and ED-3. The display device DD may include multiple light emitting elements ED-1, ED-2 and ED-3. The optical layer PP may be provided on the display panel DP and control reflected light by external light at the display panel DP. The optical layer PP may include, for example, a polarization layer and/or a color filter layer. In some embodiments, the optical layer PP may not be provided (e.g., may be omitted) in the display device DD of one or more embodiments.

On the optical layer PP, a base substrate BL may be provided. The base substrate BL may be a member providing a base surface where the optical layer PP is provided. The base substrate BL may be a glass substrate, a metal substrate, a plastic substrate, etc. However, one or more embodiments of the present disclosure is not limited thereto, and the base substrate BL may be an inorganic layer, an organic layer, or a composite material layer (e.g., including an organic material and an inorganic material). In some embodiments, the base substrate BL may not be provided (e.g., may be omitted).

The display device DD according to one or more embodiments may further include a plugging layer. The plugging layer may be provided between a display element layer DP-ED and a base substrate BL. The plugging layer may be an organic layer. The plugging layer may include at least one selected from an acrylic-based resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 provided in the pixel definition layer PDL, and an encapsulating layer TFE provided on the light emitting elements ED-1, ED-2 and ED-3.

The base layer BS may be a member providing a base surface where the display element layer DP-ED is provided. The base layer BS may be a glass substrate, a metal substrate, a plastic substrate, etc. However, one or more embodiments of the present disclosure is not limited thereto, and the base layer BS may be an inorganic layer, an organic layer, or a composite material layer.

In one or more embodiments, the circuit layer DP-CL is provided on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

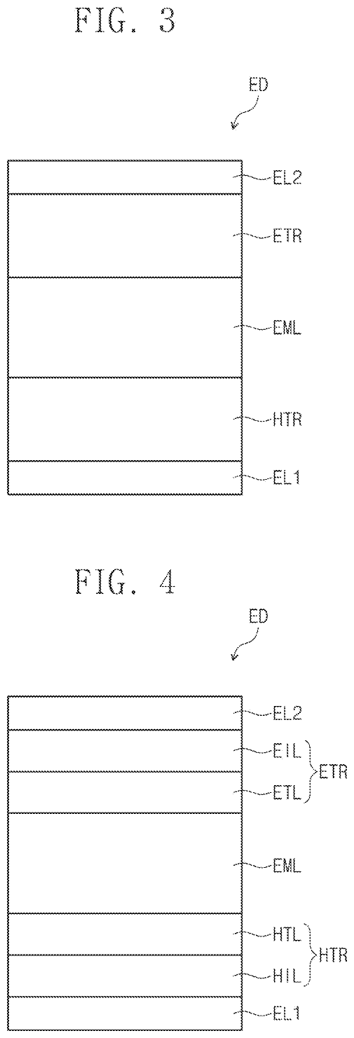

The light emitting elements ED-1, ED-2 and ED-3 may have the structures of the light emitting element ED of embodiments according to FIG. 3 to FIG. 6, which will be explained in more detail herein below. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, the emission layers EML-R, EML-G and EML-B of light emitting elements ED-1, ED-2 and ED-3 are provided in opening portions OH defined in a pixel definition layer PDL, and a hole transport region HTR, an electron transport region ETR and a second electrode EL2 are provided as common layers in (e.g., across) all light emitting elements ED-1, ED-2 and ED-3. However, one or more embodiments of the present disclosure is not limited thereto. In some embodiments, the hole transport region HTR and the electron transport region ETR may be patterned and provided in the opening portions OH defined in the pixel definition layer PDL. For example, in one or more embodiments, the hole transport region HTR, the emission layers EML-R, EML-G and EML-B, and the electron transport region ETR of the light emitting elements ED-1, ED-2 and ED-3 may be patterned by an inkjet printing method and provided.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to one or more embodiments may include at least one inorganic layer (hereinafter, encapsulating inorganic layer). In one or more embodiments, the encapsulating layer TFE according to one or more embodiments may include at least one organic layer (hereinafter, encapsulating organic layer) and at least one encapsulating inorganic layer.

The encapsulating inorganic layer may protect the display element layer DP-ED from moisture/oxygen, and the encapsulating organic layer may protect the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic-based compound, an epoxy-based compound, etc. The encapsulating organic layer may include a photopolymerizable organic material, without specific limitation.

The encapsulating layer TFE may be provided on the second electrode EL2 and may be provided while filling (e.g., to fill) the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting (e.g., configured to emit) light produced from the light emitting elements ED-1, ED-2 and ED-3, respectively. The luminous areas PXA-R, PXA-G and PXA-B may be separated from each other on a plane (e.g., in a plan view).

Each of the luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In the disclosure, each of the luminous areas PXA-R, PXA-G and PXA-B may correspond to each pixel, respectively. The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display device DD of one or more embodiments, shown in FIG. 1 and FIG. 2, three luminous areas PXA-R, PXA-G and PXA-B emitting (e.g., configured to emit) red light, green light and blue light are illustrated as one or more embodiments. For example, the display device DD of one or more embodiments may include a red luminous area PXA-R, a green luminous area PXA-G and a blue luminous area PXA-B, which are separated from each other.

In the display device DD according to one or more embodiments, multiple light emitting elements ED-1, ED-2 and ED-3 may emit light having different wavelength regions. For example, in one or more embodiments, the display device DD may include a first light emitting element ED-1 emitting (e.g., configured to emit) red light, a second light emitting element ED-2 emitting (e.g., configured to emit) green light, and a third light emitting element ED-3 emitting (e.g., configured to emit) blue light. For example, each of the red luminous area PXA-R, the green luminous area PXA-G, and the blue luminous area PXA-B of the display device DD may correspond to the first light emitting element ED-1, the second light emitting element ED-2, and the third light emitting element ED-3, respectively.

However, one or more embodiments of the present disclosure is not limited thereto, and the first to third light emitting elements ED-1, ED-2 and ED-3 may emit light in the same wavelength region, or at least one thereof may emit light in a different wavelength region. For example, all the first to third light emitting elements ED-1, ED-2 and ED-3 may emit blue light.

The luminous areas PXA-R, PXA-G and PXA-B in the display device DD according to one or more embodiments may be arranged in a stripe shape. Referring to FIG. 1, multiple red luminous areas PXA-R may be arranged with each other along a second directional axis DR2, multiple green luminous areas PXA-G may be arranged with each other along the second directional axis DR2, and multiple blue luminous areas PXA-B may be arranged with each other along the second directional axis DR2. In addition, the red luminous area PXA-R, the green luminous area PXA-G and the blue luminous area PXA-B may be arranged by turns (e.g., alternatingly with each other) along a first directional axis DR1.

In FIG. 1 and FIG. 2, the areas of the luminous areas PXA-R, PXA-G and PXA-B are shown to be similar, but one or more embodiments of the present disclosure is not limited thereto. The areas of the luminous areas PXA-R, PXA-G and PXA-B may be different from each other according to the wavelength region of light emitted. Herein, the areas of the luminous areas PXA-R, PXA-G and PXA-B may mean areas on a plane (e.g., in a plan view) defined by the first directional axis DR1 and the second directional axis DR2.

The arrangement type (or order) of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various suitable combinations according to the properties of display quality required (or desired) for the display device DD. For example, the arrangement type (or order) of the luminous areas PXA-R, PXA-G and PXA-B may be a pentile (PENTILE®) arrangement, or a diamond (Diamond Pixel™) arrangement (PENTILE® is a registered trademark owned by Samsung Display Co., Ltd.).

In some embodiments, the areas of the luminous areas PXA-R, PXA-G and PXA-B may be different from each other. For example, in one or more embodiments, the area of the green luminous area PXA-G may be smaller than the area of the blue luminous area PXA-B, but one or more embodiments of the present disclosure is not limited thereto.

Hereinafter, FIG. 3 to FIG. 7 are cross-sectional views schematically showing light emitting elements according to embodiments. The light emitting element ED according to one or more embodiments may include a first electrode EL1, a second electrode EL2 oppositely provided to the first electrode EL1, and at least one functional layer provided between the first electrode EL1 and the second electrode EL2. The light emitting element ED of one or more embodiments may include the amine compound of one or more embodiments, which will be explained in more detail herein below, in the at least one functional layer.

The light emitting element ED of one or more embodiments may include a hole transport region HTR, an emission layer EML, an electron transport region ETR, etc., as the at least one functional layer. Referring to FIG. 3, the light emitting element ED of one or more embodiments may include a first electrode EL1, a hole transport region HTR, an emission layer EML, an electron transport region ETR, and a second electrode EL2, stacked in this order.

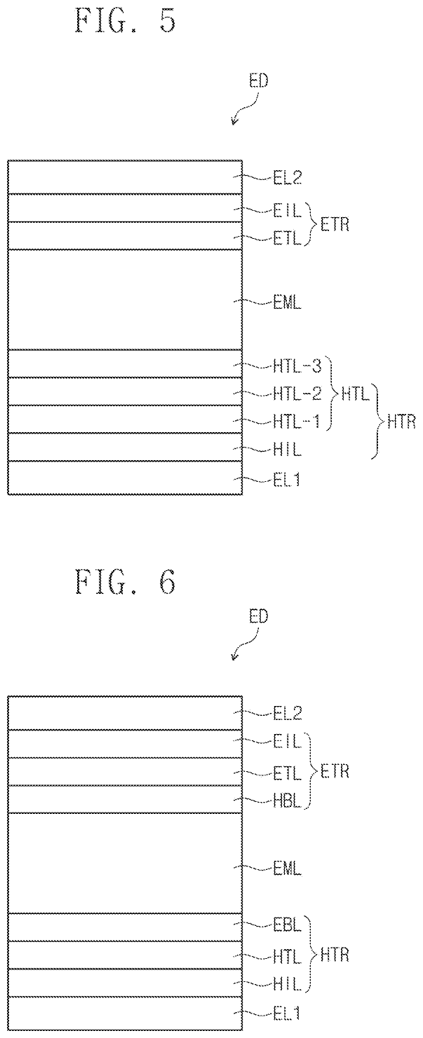

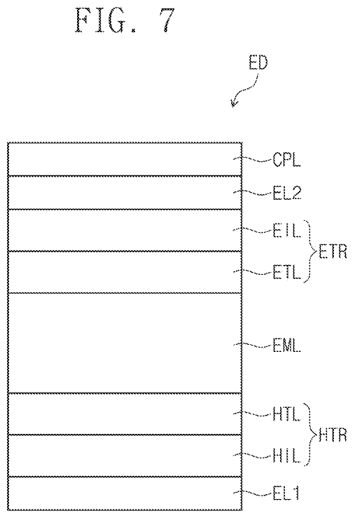

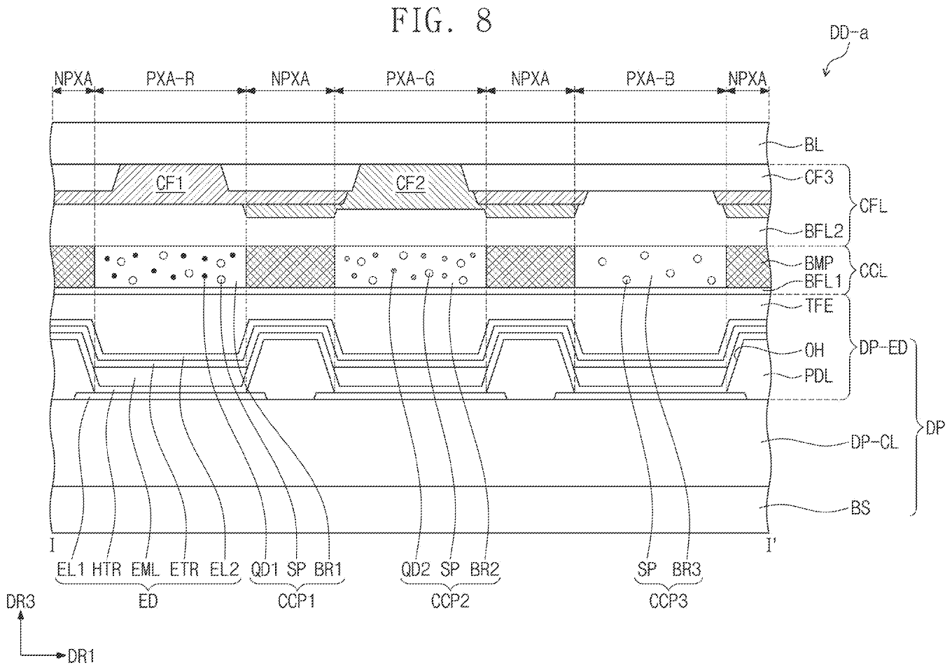

When compared to FIG. 3, FIG. 4 shows the cross-sectional view of a light emitting element ED of one or more embodiments, wherein a hole transport region HTR includes a hole injection layer HIL and a hole transport layer HTL, and an electron transport region ETR includes an electron injection layer EIL and an electron transport layer ETL. When compared to FIG. 4, FIG. 5 shows the cross-sectional view of a light emitting element ED of one or more embodiments, wherein a hole transport layer HTL includes a stacked structure of multiple hole transport layers. FIG. 5 shows a light emitting element ED of one or more embodiments, including first to third hole transport layers HTL-1, HTL-2 and HTL-3 as an illustration. When compared to FIG. 3, FIG. 6 shows the cross-sectional view of a light emitting element ED of one or more embodiments, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared to FIG. 4, FIG. 7 shows the cross-sectional view of a light emitting element ED of one or more embodiments, including a capping layer CPL provided on the second electrode EL2.

In the light emitting element ED according to one or more embodiments, the first electrode EL1 has conductivity. The first electrode EL1 may be formed using a metal material, a metal alloy or a suitable conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from Ag, Mg, Cu, Al, Pt, Pd, Au, Ni, Nd, Ir, Cr, Li, Ca, LiF, Mo, Ti, W, In, Sn, Zn, compounds of two or more selected from Ag, Mg, Cu, Al, Pt, Pd, Au, Ni, Nd, Ir, Cr, Li, Ca, LiF, Mo, Ti, W, In, Sn, and Zn, mixtures of two or more selected from Ag, Mg, Cu, Al, Pt, Pd, Au, Ni, Nd, Ir, Cr, Li, Ca, LiF, Mo, Ti, W, In, Sn, and Zn, and oxides of Ag, Mg, Cu, Al, Pt, Pd, Au, Ni, Nd, Ir, Cr, Li, Ca, LiF, Mo, Ti, W, In, Sn, and Zn.

If the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compound(s) thereof, and/or mixture(s) thereof (for example, a mixture of Ag and Mg). Also, the first electrode EL1 may have a structure including multiple layers including a reflective layer or a transflective layer formed using any of the above materials, and a transmissive conductive layer formed using ITO, IZO, ZnO, and/or ITZO. For example, the first electrode EL1 may have a three-layer structure of ITO/Ag/ITO. In some embodiments, the first electrode EL1 may include the above-described metal materials, combinations of two or more metal materials selected from the above-described metal materials, and/or oxides of the above-described metal materials. However, one or more embodiments of the present disclosure is not limited thereto. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer, an emission auxiliary layer, or an electron blocking layer EBL.

The hole transport region HTR may have a single layer formed using a single material, a single layer formed using multiple different materials, or a multilayer structure including multiple layers formed using multiple different materials. For example, the hole transport region HTR may have the structure of a single layer of a hole injection layer HIL or a hole transport layer HTL, and may have a structure of a single layer formed using a hole injection material and a hole transport material.

In some embodiments, the hole transport region HTR may have a structure of a single layer formed using multiple different materials, or a structure stacked from the first electrode EL1 of hole injection layer HIL/hole transport layer HTL, hole injection layer HIL/first hole transport layer HTL-1/second hole transport layer HTL-2/third hole transport layer HTL-3, hole injection layer HIL/hole transport layer HTL/buffer layer, hole injection layer HIL/buffer layer, or hole transport layer HTL/buffer layer, without limitation.

If the hole transport region HTR includes the first to third hole transport layers HTL-1, HTL-2 and HTL-3, at least one selected from the first to third hole transport layers HTL-1, HTL-2 and HTL-3 may include the amine compound of one or more embodiments. For example, the first and third hole transport layers HTL-1, and HTL-3 may include the amine compound of one or more embodiments. The first and third hole transport layers HTL-1 and HTL-3 may include the same material, and the second hole transport layer HTL-2 may include a different material from that of the first and third hole transport layers HTL-1 and HTL-3. However, these are illustrations, and one or more embodiments of the present disclosure is not limited thereto.

The thickness of the hole transport region HTR may be, for example, about 50 Å to about 15,000 Å. The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of one or more embodiments may include the amine compound of one or more embodiments in a hole transport region HTR. The light emitting element ED of one or more embodiments may include the amine compound of one or more embodiments in at least one selected from a hole injection layer HIL and a hole transport layer HTL. In some embodiments, the light emitting element ED may include the amine compound of one or more embodiments in at least one selected from the hole injection layer HIL, the hole transport layer HTL, and the electron blocking layer EBL of a hole transport region HTR. For example, the light emitting element ED of one or more embodiments may include the amine compound of one or more embodiments in a hole transport layer HTL. However, one or more embodiments of the present disclosure is not limited thereto, and the light emitting element ED of one or more embodiments may include the amine compound of one or more embodiments in a hole injection layer HIL and/or an electron blocking layer EBL.

The amine compound of one or more embodiments may include a linker of two spiro-bonded hydrocarbon rings, and two amine groups connected to the linker. In the amine compound of one or more embodiments, the linker may have a spiro-bonded structure of a first hydrocarbon ring and a second hydrocarbon ring. In one or more embodiments, the first hydrocarbon ring and the second hydrocarbon ring may be the same. Each of the first and second hydrocarbon rings may have a fused structure of one benzene ring and one cycloalkyl group. For example, the first and second hydrocarbon rings may have a fused structure of a benzene ring and cyclopentane, a fused structure of a benzene ring and cyclohexane, or a fused structure of a benzene ring and cycloheptane. The amine compound of one or more embodiments may include a spiro-bonded linker of cycloalkyl group of the first hydrocarbon ring and cycloalkyl group of the second hydrocarbon ring.

The amine compound of one or more embodiments includes a first amine group and a second amine group, connected with the linker. The first amine group and the second amine group may be connected with the benzene ring of the first hydrocarbon ring and the benzene ring of the second hydrocarbon ring, respectively. For example, the first amine group may be connected with the benzene ring of the first hydrocarbon ring, and the second amine group may be connected with the benzene ring of the second hydrocarbon ring. The first amine group and the second amine group may each independently make a direct linkage with the linker, or may be bonded thereto with an additional linker therebetween. In one or more embodiments, the first amine group and the second amine group may be the same or different from each other.

The amine compound of one or more embodiments may be represented by Formula 1 below.

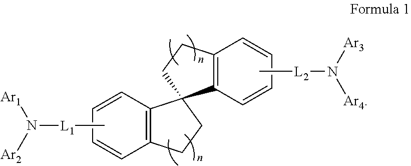

In Formula 1, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. For example, L₁ and L₂ may be each independently a direct linkage, or a substituted or unsubstituted phenyl group. However, one or more embodiments of the present disclosure is not limited thereto.

In Formula 1, “n” may be an integer of 1 to 3. For example, “n” may be 1 or 2. However, one or more embodiments of the present disclosure is not limited thereto, and “n” may be 3. If “n” is 1, the amine compound of one or more embodiments may include a spiro-bonded linker of two indane groups. If “n” is 2, the amine compound of one or more embodiments may include a spiro-bonded linker of two tetralin groups. If “n” is 3, the amine compound of one or more embodiments may include a spiro-bonded linker of two benzocycloheptene groups.

In Formula 1, Ar₁ to Ar₄ may be each independently a substituted or unsubstituted hydrocarbon ring of 6 to 30 ring-forming carbon atoms, or a substituted or unsubstituted heteroaryl group of 2 to 30 ring-forming carbon atoms. For example, Ar₁ to Ar₄ may be each independently a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted benzofluorene group, a substituted or unsubstituted dibenzofuran group, a substituted or unsubstituted benzonaphthofuran group, a substituted or unsubstituted dibenzothiophene group, a substituted or unsubstituted benzonaphthothiophene group, a substituted or unsubstituted carbazole group, a substituted or unsubstituted benzocarbazole group, a substituted or unsubstituted cyclohexylbenzene group, or a substituted or unsubstituted tetrahydronaphthyl group. However, one or more embodiments of the present disclosure is not limited thereto. In Formula 1, an amine group substituted with Ar₁ and Ar₂ corresponds to the first amine group, and an amine group substituted with Ar₃ and Ar₄ corresponds to the second amine group.

In one or more embodiments, Ar₁ to Ar₄ may be each independently represented by Formula 1-1a or Formula 1-1b. For example, any one selected from Ar₁ to Ar₄ may be represented by Formula 1-1a, and the remainder may be represented by Formula 1-1b. In some embodiments, Ar₁ to Ar₄ may be all represented by Formula 1-1b. However, one or more embodiments of the present disclosure is not limited thereto, and Ar₁ to Ar₄ may be all represented by Formula 1-1a.

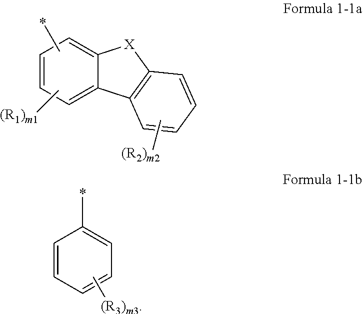

In Formula 1-1a, X may be O, S, CR_{X}R_{y}, or NR_{z}. For example, if Formula 1-1a is bonded to the nitrogen atoms of the first amine group and/or the second amine group, the amine compound of one or more embodiments may include the bonded substituent of a substituted or unsubstituted dibenzofuran moiety, a substituted or unsubstituted dibenzothiophene moiety, a substituted or unsubstituted fluorenyl moiety, or a substituted or unsubstituted carbazole moiety.

In Formula 1-1a, Rₓ, R_{y}, and R_{z} may be each independently a hydrogen atom, a deuterium atom, a substituted or unsubstituted alkyl group of 1 to 10 carbon atoms, or a substituted or unsubstituted aryl group of 6 to 15 ring-forming carbon atoms. For example, Rₓ, R_{y}, and R_{z} may be each independently a hydrogen atom, a deuterium atom, a substituted or unsubstituted methyl group, or a substituted or unsubstituted phenyl group.

In Formula 1-1a and Formula 1-1b, R₁ to R₃ may be each independently a hydrogen atom, a deuterium atom, a substituted or unsubstituted alkyl group of 1 to 10 carbon atoms, a substituted or unsubstituted cycloalkyl group of 6 to 15 ring-forming carbon atoms, or a substituted or unsubstituted aryl group of 6 to 15 ring-forming carbon atoms. For example, R₁ to R₃ may be each independently a hydrogen atom, a deuterium atom, a substituted or unsubstituted isopropyl group, a substituted or unsubstituted t-butyl group, a substituted or unsubstituted cyclohexyl group, a substituted or unsubstituted phenyl group, a substituted or unsubstituted biphenyl group, or a substituted or unsubstituted naphthyl group. In some embodiments, R₁ to R₃ may be each independently combined with an adjacent group to form a ring. For example, adjacent two R₁ may be combined with each other to form a substituted or unsubstituted hydrocarbon ring, or adjacent two R₂ may be combined with each other to form a substituted or unsubstituted hydrocarbon ring. In some embodiments, adjacent two R₃ may be combined with each other to form a substituted or unsubstituted hydrocarbon ring.

In Formula 1-1a and Formula 1-1b, m1 is an integer of 0 to 3, m2 is an integer of 0 to 4, and m3 is an integer of 0 to 5. If m1 is 0, the amine compound of one or more embodiments may be unsubstituted with R₁. A case where m1 is 3, and all R₁ are hydrogen atoms, may be the same as a case where m1 is 0. If m2 is 0, the amine compound of one or more embodiments may be unsubstituted with R₂. A case where m2 is 4, and all R₂ are hydrogen atoms, may be the same as a case where m2 is 0. If m3 is 0, the amine compound of one or more embodiments may be unsubstituted with R₃. A case where m3 is 5, and all R₃ are hydrogen atoms, may be the same as a case where m3 is 0. If each of m1, m2, and m3 is an integer of 2 or more, multiple R₁, R₂, and R₃ may be each independently the same, or at least one may be different from the remainder. In Formula 1-1a and Formula 1-1b, “-*” is a position connected with the nitrogen atom in Formula 1.

In one or more embodiments, the amine compound represented by Formula 1 may be represented by Formula 2-1 or Formula 2-2. Formula 2-1 corresponds to Formula 1 where “n” is 1, and Formula 2-2 corresponds to Formula 1 where “n” is 2. For example, the amine compound of one or more embodiments may include a spiro-bonded linker of two indane moieties, or a spiro-bonded linker of two tetralin, connecting the first amine group and the second amine group. In Formula 2-1 and Formula 2-2, the same descriptions as those provided in connection with Formula 1 may be applied for Ar₁ to Ar₄, L₁ and L₂.

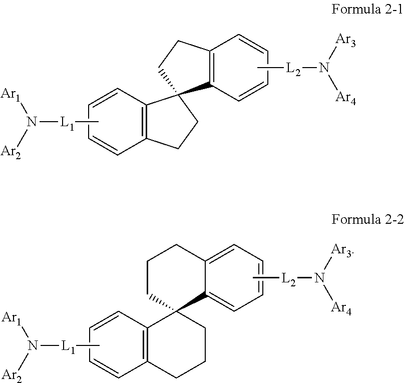

In one or more embodiments, the amine compound represented by Formula 1 may be represented by Formula 3-1 or Formula 3-2. Each of Formula 3-1 and Formula 3-2 represents Formula 1 where the bonding positions of the first amine group connected with L₁ and the second amine group connected with L₂ are specified in Formula 1. In Formula 3-1 or Formula 3-2, the same descriptions as those provided in connection with Formula 1 may be applied for Ar₁ to Ar₄, L₁, L₂, and “n”.

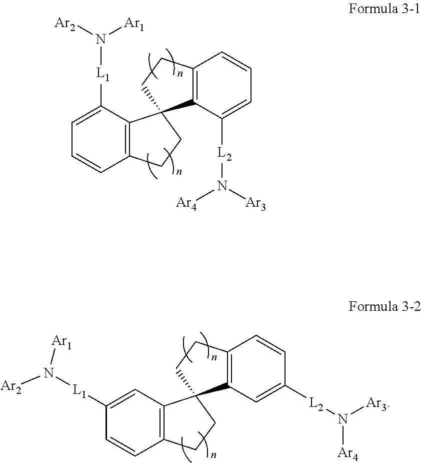

In one or more embodiments, the amine compound represented by Formula 1 may be represented by Formula 4-1. Formula 4-1 represents a case of Formula 1 where L₁ and L₂ are embodied. Formula 4-1 corresponds to a case where both L₁ and L₂ are direct linkages. For example, in the amine compound of one or more embodiments, represented by Formula 4-1, the first amine group and the second amine group may be directly bonded to a spiro-bonded linker of two hydrocarbon rings. In Formula 4-1, the same descriptions as those provided in connection with Formula 1 may be applied for Ar₁ to Ar₄, and “n”.

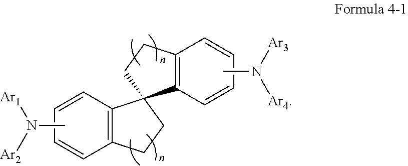

In one or more embodiments, the amine compound represented by Formula 1 may be represented by Formula 5-1 or Formula 5-2. Formula 5-1 and Formula 5-2 are amine compounds of embodiments of Formula 1 wherein substituents substituted at the first amine group and the second amine group, are specified. Formula 5-1 corresponds to a case of Formula 1 where any one selected from Ar₁ to Ar₄ is represented by Formula 1-1a above, and the remainders are represented by Formula 1-1b above. Formula 5-2 corresponds to a case where Ar₁ to Ar₄ are all represented by Formula 1-1b. In Formula 5-1 and 5-2, the same descriptions as those provided in connection with Formula 1 may be applied for L₁, L₂, and “n”.

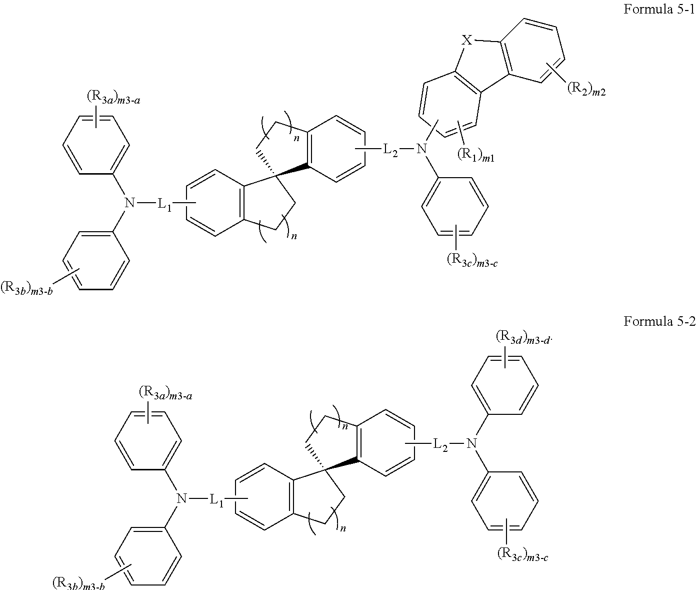

In one or more embodiments, the same description for X as provided in connection with Formula 1-1a may be applied for X in Formula 5-1. In some embodiments, the same descriptions for m1, m2, R₁ and R₂ as provided in connection with Formula 1-1a may be applied for m1, m2, R₁ and R₂, in Formula 5-1, respectively.

In Formula 5-1 and Formula 5-2, R₃ₐ, R_{3b}, R_{3c}, and R_{3d} may be each independently a hydrogen atom, a deuterium atom, a substituted or unsubstituted alkyl group of 1 to 10 carbon atoms, a substituted or unsubstituted cycloalkyl group of 6 to 15 ring-forming carbon atoms, or a substituted or unsubstituted aryl group of 6 to 15 ring-forming carbon atoms. In some embodiments, R₃ₐ, R_{3b}, R_{3c}, and R_{3d} may be each independently combined with an adjacent group to form a ring. The same description as provided for R₃ in connection with Formula 1-1b may be applied for R₃ₐ, R_{3b}, R_{3c}, and R_{3d}.

In Formula 5-1 and Formula 5-2, m3-a, m3-b, m3-c, and m3-d may be each independently an integer of 0 to 5. The same description as provided for m3 in connection with Formula 1-1b may be applied for m3-a, m3-b, m3-c, and m3-d.

In one or more embodiments, the amine compound represented by Formula 5-1 may be represented by Formula 5-1a or Formula 5-1b. Each of Formula 5-1a and Formula 5-1b specifies the bonding position of Formula 1-1a which is bonded to the nitrogen atom of Formula 1. Each of Formula 5-1a and Formula 5-1b corresponds to a case of Formula 5-1 where Ar₁ to Ar₄ are embodied. In Formula 5-1a and Formula 5-1b, the same descriptions as those provided in connection with Formula 5-1 may be applied for L₁, L₂, X, R₁, R₂, R₃ₐ, R_{3b}, R_{3c}, “n”, m1, m2, m3-a, m3-b, and m3-c. For example, in Formula 5-1a and Formula 5-1b, the same descriptions as those provided in connection with Formula 1 may be applied for L₁, L₂, and “n”. In some embodiments, in Formula 5-1a and Formula 5-1b, the same descriptions as those provided in connection with Formula 1-1a may be applied for X, R₁, R₂, m1 and m2, and the same descriptions as those provided in connection with Formula 1-1b may be applied for R₃ₐ, R_{3b}, R_{3c}, and R_{3d}. The same description as provided for m3 in connection with in Formula 1-1b may be applied for m3-a, m3-b, m3-c, and m3-d.

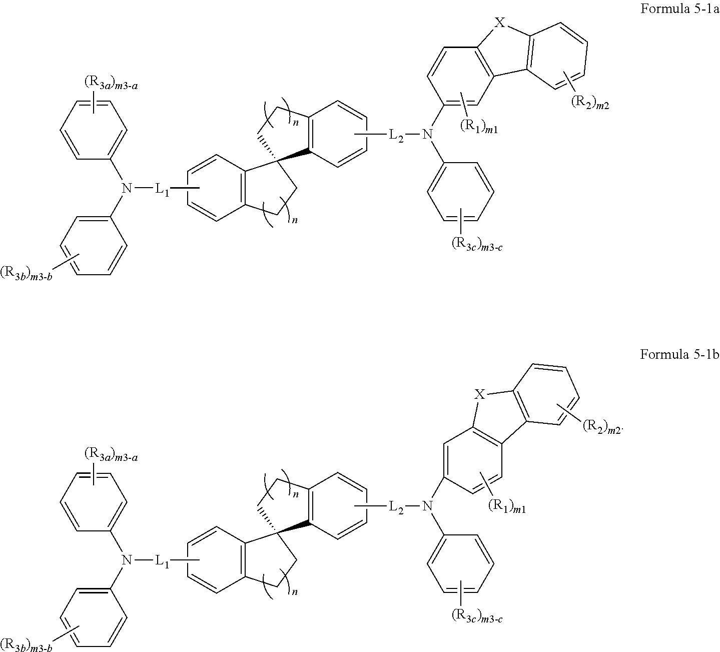

The amine compound represented by Formula 1 may be represented by any one selected from compounds in Compound Group 1. The light emitting element ED of one or more embodiments may include at least one selected from the amine compounds shown in Compound Group 1 in a hole transport region HTR.

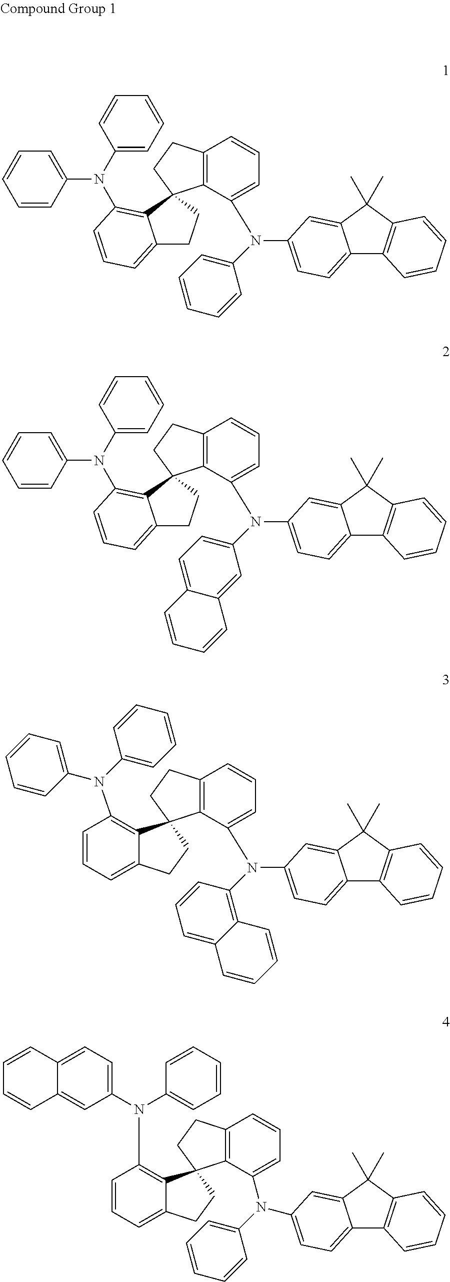 [SMILES: CC1(C)c2ccccc2-c2ccc(N(c3ccccc3)c3cccc4c3[C@]3(CCc5cccc(N(c6ccccc6)c6ccccc6)c53)CC4)cc21 ; CC1(C)c2ccccc2-c2ccc(N(c3ccc4ccccc4c3)c3cccc4c3[C@]3(CCc5cccc(N(c6ccccc6)c6ccccc6)c53)CC4)cc21 ; CC1(C)c2ccccc2-c2ccc(N(c3cccc4c3[C@]3(CCc5cccc(N(c6ccccc6)c6ccccc6)c53)CC4)c3cccc4ccccc34)cc21 ; CC1(C)c2ccccc2-c2ccc(N(c3ccccc3)c3cccc4c3[C@@]3(CC4)CCc4cccc(N(c5ccccc5)c5ccc6ccccc6c5)c43)cc21]

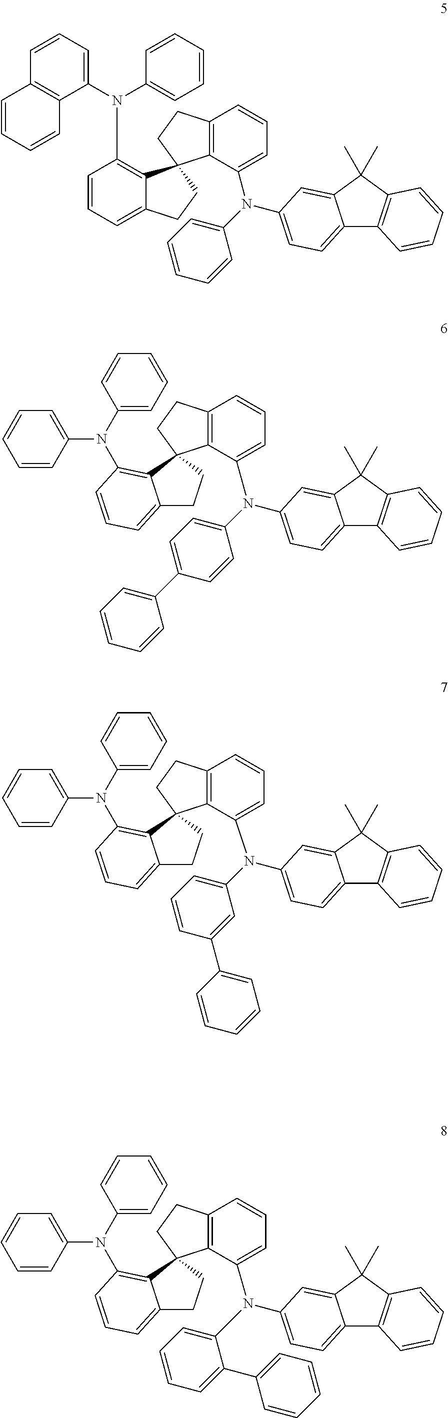 [SMILES: CC1(C)c2ccccc2-c2ccc(N(c3ccccc3)c3cccc4c3[C@@]3(CC4)CCc4cccc(N(c5ccccc5)c5cccc6ccccc56)c43)cc21 ; CC1(C)c2ccccc2-c2ccc(N(c3ccc(-c4ccccc4)cc3)c3cccc4c3[C@]3(CCc5cccc(N(c6ccccc6)c6ccccc6)c53)CC4)cc21 ; CC1(C)c2ccccc2-c2ccc(N(c3cccc(-c4ccccc4)c3)c3cccc4c3[C@]3(CCc5cccc(N(c6ccccc6)c6ccccc6)c53)CC4)cc21 ; CC1(C)c2ccccc2-c2ccc(N(c3ccccc3-c3ccccc3)c3cccc4c3[C@]3(CCc5cccc(N(c6ccccc6)c6ccccc6)c53)CC4)cc21]

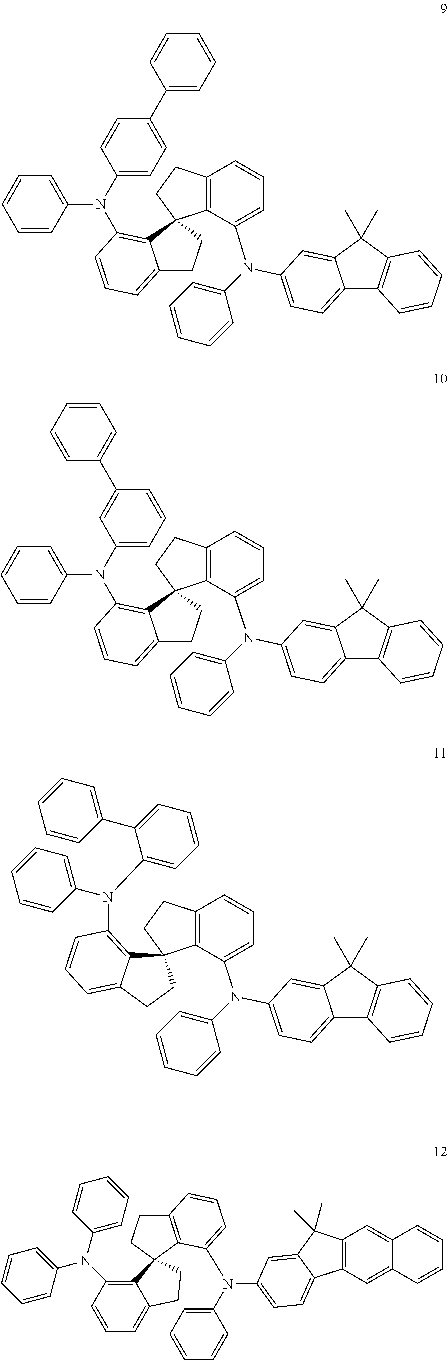 [SMILES: CC1(C)c2ccccc2-c2ccc(N(c3ccccc3)c3cccc4c3[C@]3(CCc5cccc(N(c6ccccc6)c6ccc(-c7ccccc7)cc6)c53)CC4)cc21 ; CC1(C)c2ccccc2-c2ccc(N(c3ccccc3)c3cccc4c3[C@]3(CCc5cccc(N(c6ccccc6)c6cccc(-c7ccccc7)c6)c53)CC4)cc21 ; CC1(C)c2ccccc2-c2ccc(N(c3ccccc3)c3cccc4c3[C@@]3(CC4)CCc4cccc(N(c5ccccc5)c5ccccc5-c5ccccc5)c43)cc21 ; CC1(C)c2cc(N(c3ccccc3)c3cccc4c3[C@]3(CCc5cccc(N(c6ccccc6)c6ccccc6)c53)CC4)ccc2-c2cc3ccccc3cc21]

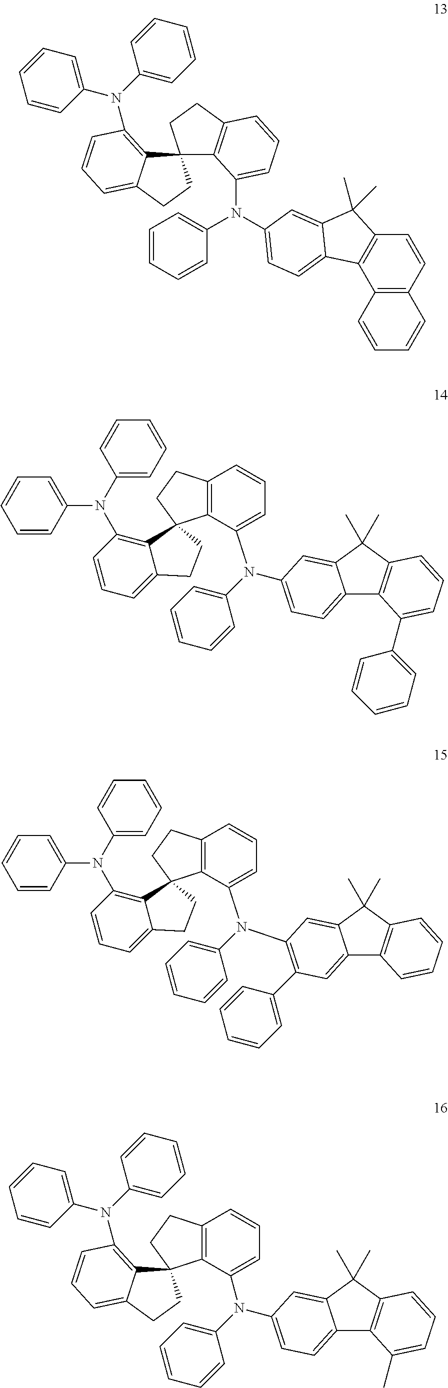 [SMILES: CC1(C)c2cc(N(c3ccccc3)c3cccc4c3[C@]3(CCc5cccc(N(c6ccccc6)c6ccccc6)c53)CC4)ccc2-c2c1ccc1ccccc21 ; CC1(C)c2cc(N(c3ccccc3)c3cccc4c3[C@]3(CCc5cccc(N(c6ccccc6)c6ccccc6)c53)CC4)ccc2-c2c(-c3ccccc3)cccc21 ; CC1(C)c2ccccc2-c2cc(-c3ccccc3)c(N(c3ccccc3)c3cccc4c3[C@]3(CCc5cccc(N(c6ccccc6)c6ccccc6)c53)CC4)cc21 ; Cc1cccc2c1-c1ccc(N(c3ccccc3)c3cccc4c3[C@]3(CCc5cccc(N(c6ccccc6)c6ccccc6)c53)CC4)cc1C2(C)C]

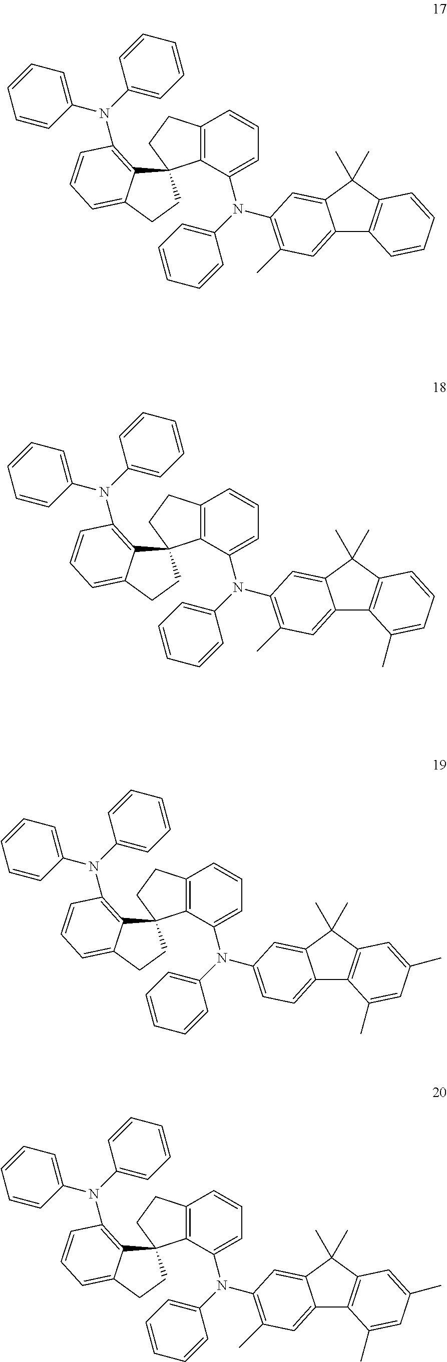 [SMILES: Cc1cc2c(cc1N(c1ccccc1)c1cccc3c1[C@]1(CCc4cccc(N(c5ccccc5)c5ccccc5)c41)CC3)C(C)(C)c1ccccc1-2 ; Cc1cc2c(cc1N(c1ccccc1)c1cccc3c1[C@]1(CCc4cccc(N(c5ccccc5)c5ccccc5)c41)CC3)C(C)(C)c1cccc(C)c1-2 ; Cc1cc(C)c2c(c1)C(C)(C)c1cc(N(c3ccccc3)c3cccc4c3[C@]3(CCc5cccc(N(c6ccccc6)c6ccccc6)c53)CC4)ccc1-2 ; Cc1cc(C)c2c(c1)C(C)(C)c1cc(N(c3ccccc3)c3cccc4c3[C@]3(CCc5cccc(N(c6ccccc6)c6ccccc6)c53)CC4)c(C)cc1-2]

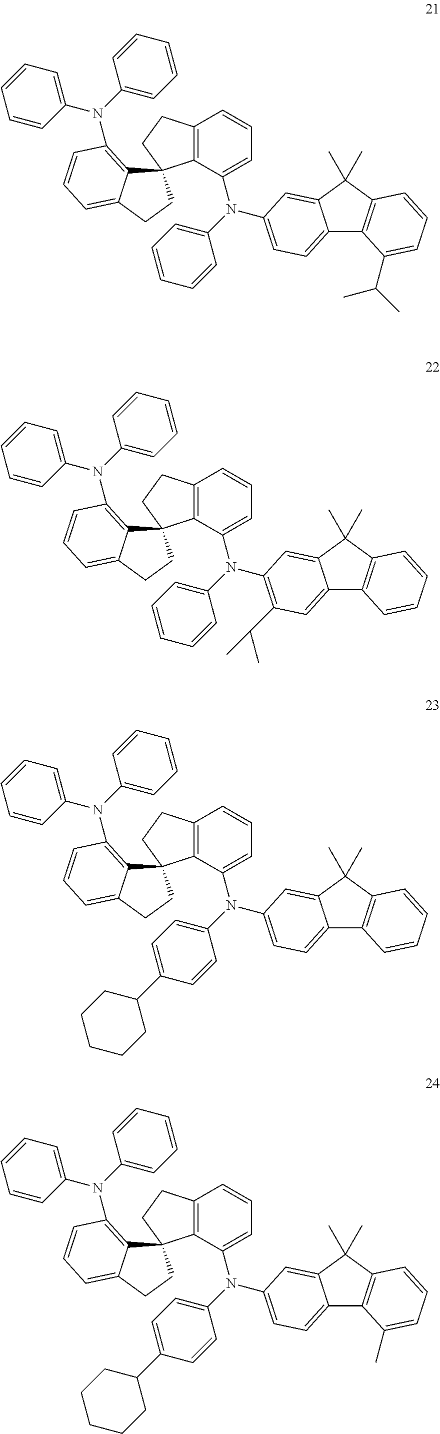 [SMILES: CC(C)c1cccc2c1-c1ccc(N(c3ccccc3)c3cccc4c3[C@]3(CCc5cccc(N(c6ccccc6)c6ccccc6)c53)CC4)cc1C2(C)C ; CC(C)c1cc2c(cc1N(c1ccccc1)c1cccc3c1[C@]1(CCc4cccc(N(c5ccccc5)c5ccccc5)c41)CC3)C(C)(C)c1ccccc1-2 ; CC1(C)c2ccccc2-c2ccc(N(c3ccc(C4CCCCC4)cc3)c3cccc4c3[C@]3(CCc5cccc(N(c6ccccc6)c6ccccc6)c53)CC4)cc21 ; Cc1cccc2c1-c1ccc(N(c3ccc(C4CCCCC4)cc3)c3cccc4c3[C@]3(CCc5cccc(N(c6ccccc6)c6ccccc6)c53)CC4)cc1C2(C)C]

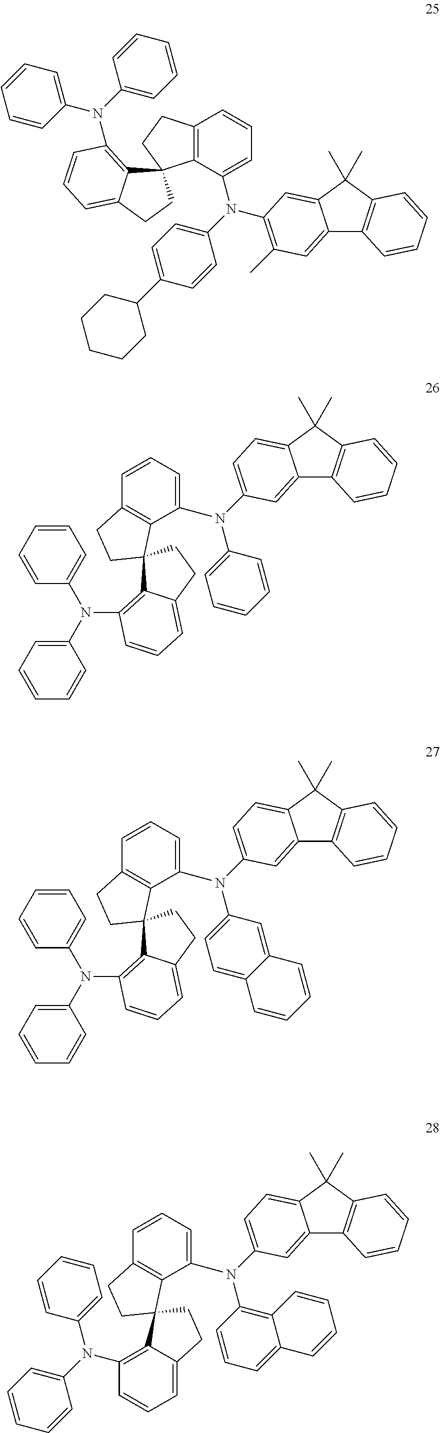 [SMILES: Cc1cc2c(cc1N(c1ccc(C3CCCCC3)cc1)c1cccc3c1[C@]1(CCc4cccc(N(c5ccccc5)c5ccccc5)c41)CC3)C(C)(C)c1ccccc1-2 ; CC1(C)c2ccccc2-c2cc(N(c3ccccc3)c3cccc4c3[C@]3(CCc5cccc(N(c6ccccc6)c6ccccc6)c53)CC4)ccc21 ; CC1(C)c2ccccc2-c2cc(N(c3ccc4ccccc4c3)c3cccc4c3[C@]3(CCc5cccc(N(c6ccccc6)c6ccccc6)c53)CC4)ccc21 ; CC1(C)c2ccccc2-c2cc(N(c3cccc4c3[C@]3(CCc5cccc(N(c6ccccc6)c6ccccc6)c53)CC4)c3cccc4ccccc34)ccc21]

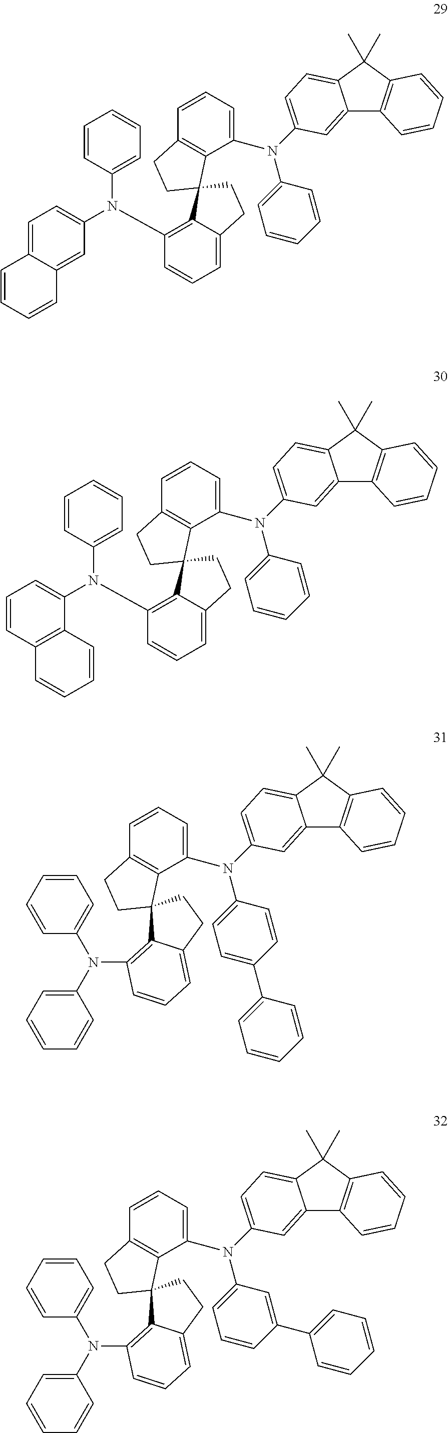 [SMILES: CC1(C)c2ccccc2-c2cc(N(c3ccccc3)c3cccc4c3[C@@]3(CC4)CCc4cccc(N(c5ccccc5)c5ccc6ccccc6c5)c43)ccc21 ; CC1(C)c2ccccc2-c2cc(N(c3ccccc3)c3cccc4c3[C@@]3(CC4)CCc4cccc(N(c5ccccc5)c5cccc6ccccc56)c43)ccc21 ; CC1(C)c2ccccc2-c2cc(N(c3ccc(-c4ccccc4)cc3)c3cccc4c3[C@]3(CCc5cccc(N(c6ccccc6)c6ccccc6)c53)CC4)ccc21 ; CC1(C)c2ccccc2-c2cc(N(c3cccc(-c4ccccc4)c3)c3cccc4c3[C@]3(CCc5cccc(N(c6ccccc6)c6ccccc6)c53)CC4)ccc21]

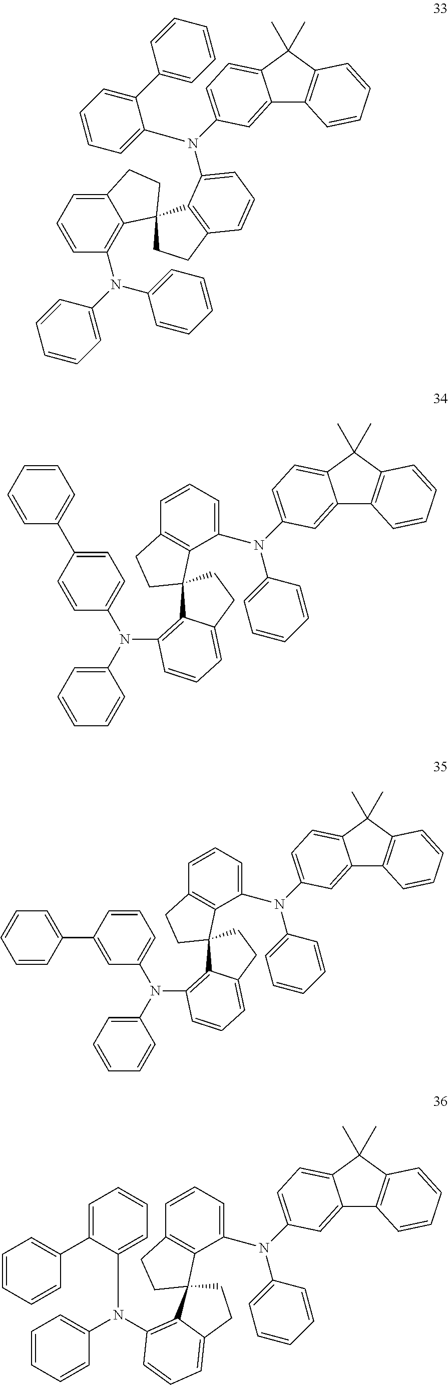 [SMILES: CC1(C)c2ccccc2-c2cc(N(c3ccccc3-c3ccccc3)c3cccc4c3[C@]3(CCc5cccc(N(c6ccccc6)c6ccccc6)c53)CC4)ccc21 ; CC1(C)c2ccccc2-c2cc(N(c3ccccc3)c3cccc4c3[C@]3(CCc5cccc(N(c6ccccc6)c6ccc(-c7ccccc7)cc6)c53)CC4)ccc21 ; CC1(C)c2ccccc2-c2cc(N(c3ccccc3)c3cccc4c3[C@]3(CCc5cccc(N(c6ccccc6)c6cccc(-c7ccccc7)c6)c53)CC4)ccc21 ; CC1(C)c2ccccc2-c2cc(N(c3ccccc3)c3cccc4c3[C@@]3(CC4)CCc4cccc(N(c5ccccc5)c5ccccc5-c5ccccc5)c43)ccc21]

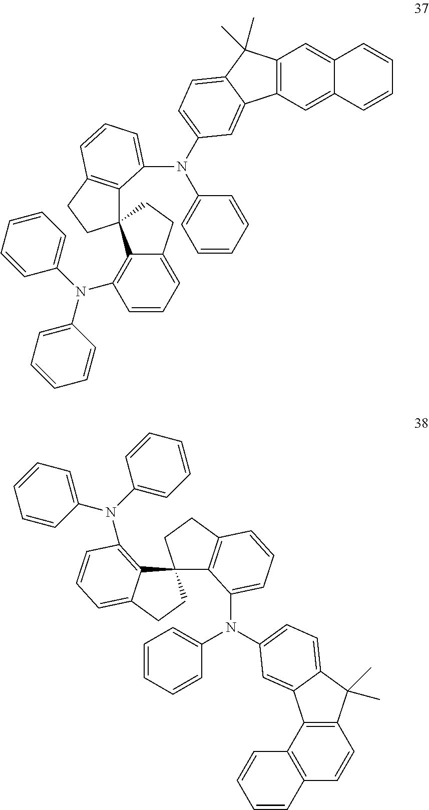 [SMILES: CC1(C)c2ccc(N(c3ccccc3)c3cccc4c3[C@]3(CCc5cccc(N(c6ccccc6)c6ccccc6)c53)CC4)cc2-c2cc3ccccc3cc21 ; CC1(C)c2ccc(N(c3ccccc3)c3cccc4c3[C@]3(CCc5cccc(N(c6ccccc6)c6ccccc6)c53)CC4)cc2-c2c1ccc1ccccc21]

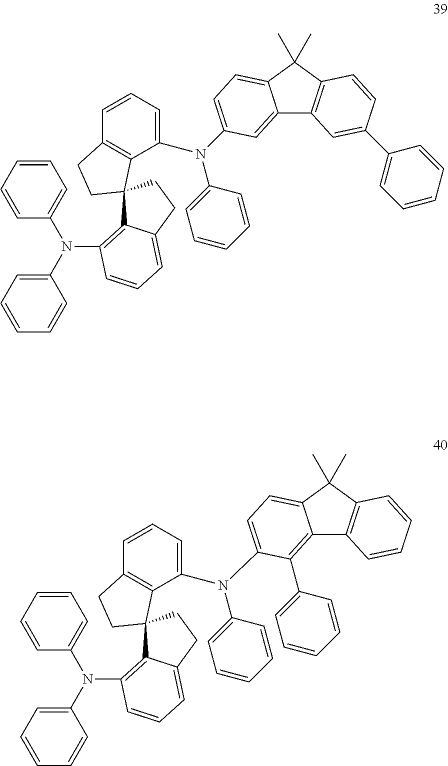 [SMILES: CC1(C)c2ccc(-c3ccccc3)cc2-c2cc(N(c3ccccc3)c3cccc4c3[C@]3(CCc5cccc(N(c6ccccc6)c6ccccc6)c53)CC4)ccc21 ; CC1(C)c2ccccc2-c2c1ccc(N(c1ccccc1)c1cccc3c1[C@]1(CCc4cccc(N(c5ccccc5)c5ccccc5)c41)CC3)c2-c1ccccc1]

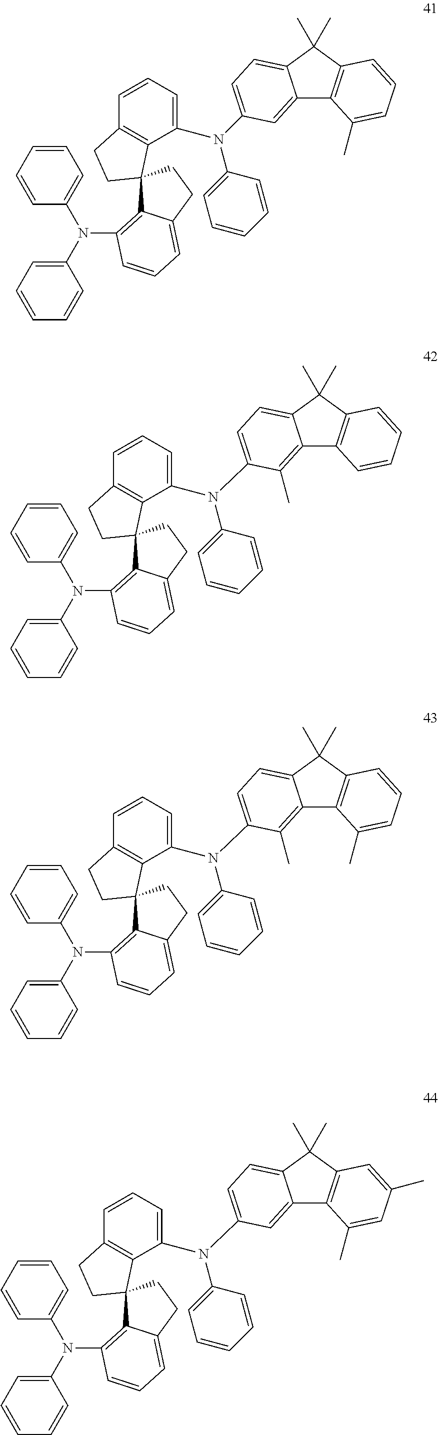 [SMILES: Cc1cccc2c1-c1cc(N(c3ccccc3)c3cccc4c3[C@]3(CCc5cccc(N(c6ccccc6)c6ccccc6)c53)CC4)ccc1C2(C)C ; Cc1c(N(c2ccccc2)c2cccc3c2[C@]2(CCc4cccc(N(c5ccccc5)c5ccccc5)c42)CC3)ccc2c1-c1ccccc1C2(C)C ; Cc1cccc2c1-c1c(ccc(N(c3ccccc3)c3cccc4c3[C@]3(CCc5cccc(N(c6ccccc6)c6ccccc6)c53)CC4)c1C)C2(C)C ; Cc1cc(C)c2c(c1)C(C)(C)c1ccc(N(c3ccccc3)c3cccc4c3[C@]3(CCc5cccc(N(c6ccccc6)c6ccccc6)c53)CC4)cc1-2]

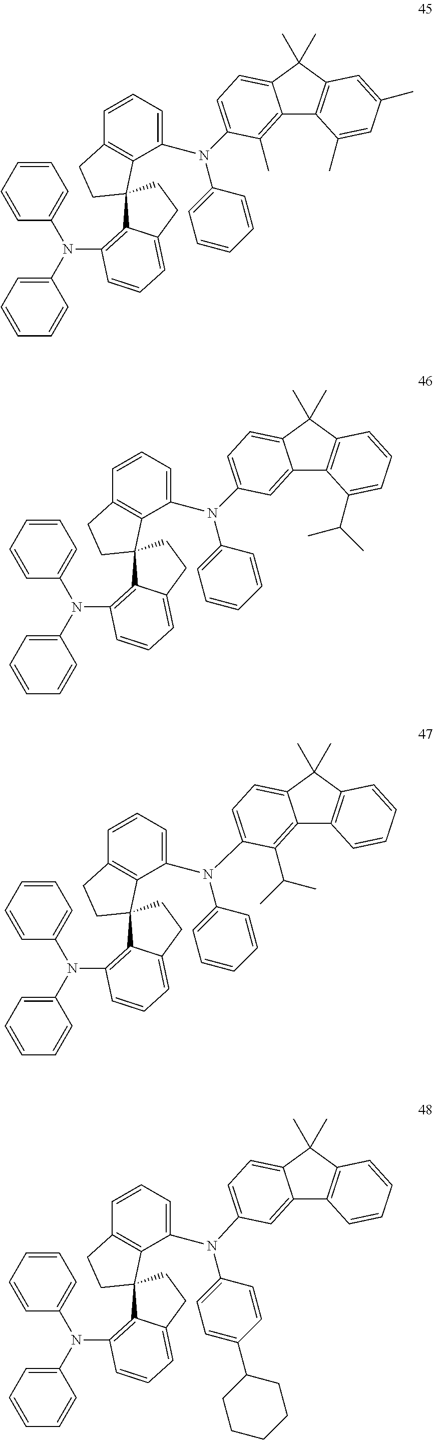 [SMILES: Cc1cc(C)c2c(c1)C(C)(C)c1ccc(N(c3ccccc3)c3cccc4c3[C@]3(CCc5cccc(N(c6ccccc6)c6ccccc6)c53)CC4)c(C)c1-2 ; CC(C)c1cccc2c1-c1cc(N(c3ccccc3)c3cccc4c3[C@]3(CCc5cccc(N(c6ccccc6)c6ccccc6)c53)CC4)ccc1C2(C)C ; CC(C)c1c(N(c2ccccc2)c2cccc3c2[C@]2(CCc4cccc(N(c5ccccc5)c5ccccc5)c42)CC3)ccc2c1-c1ccccc1C2(C)C ; CC1(C)c2ccccc2-c2cc(N(c3ccc(C4CCCCC4)cc3)c3cccc4c3[C@]3(CCc5cccc(N(c6ccccc6)c6ccccc6)c53)CC4)ccc21]

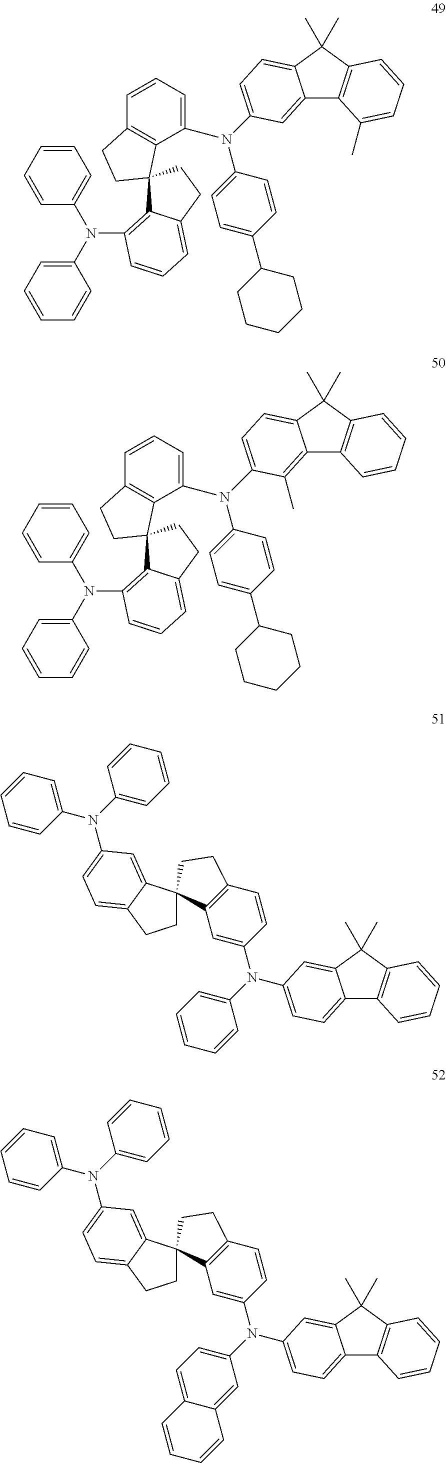 [SMILES: Cc1cccc2c1-c1cc(N(c3ccc(C4CCCCC4)cc3)c3cccc4c3[C@]3(CCc5cccc(N(c6ccccc6)c6ccccc6)c53)CC4)ccc1C2(C)C ; Cc1c(N(c2ccc(C3CCCCC3)cc2)c2cccc3c2[C@]2(CCc4cccc(N(c5ccccc5)c5ccccc5)c42)CC3)ccc2c1-c1ccccc1C2(C)C ; CC1(C)c2ccccc2-c2ccc(N(c3ccccc3)c3ccc4c(c3)[C@]3(CCc5ccc(N(c6ccccc6)c6ccccc6)cc53)CC4)cc21 ; CC1(C)c2ccccc2-c2ccc(N(c3ccc4c(c3)[C@]3(CCc5ccc(N(c6ccccc6)c6ccccc6)cc53)CC4)c3ccc4ccccc4c3)cc21]

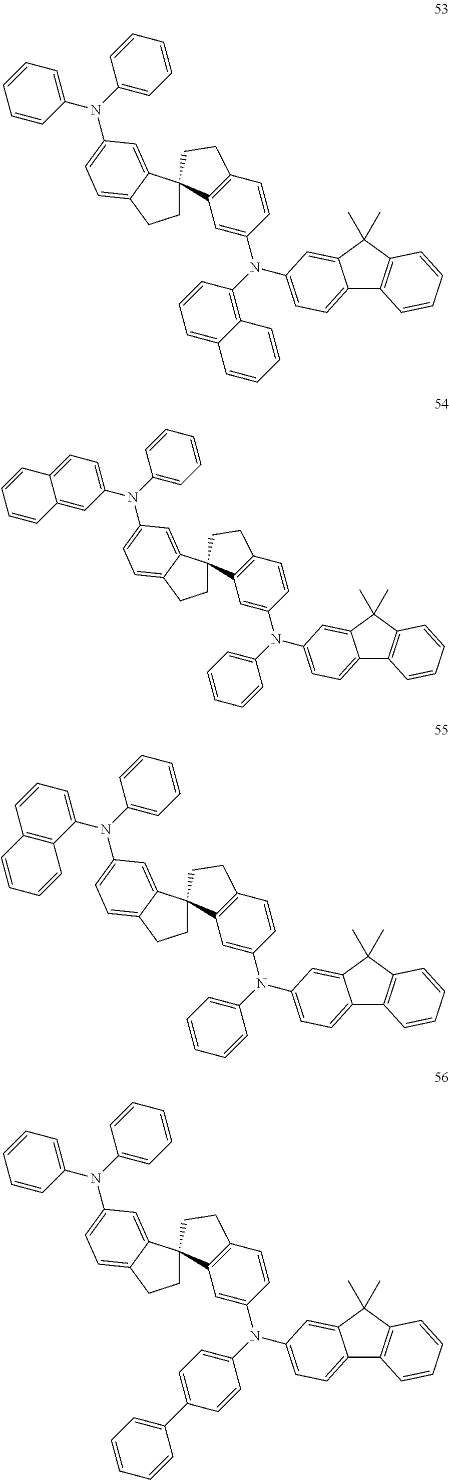 [SMILES: CC1(C)c2ccccc2-c2ccc(N(c3ccc4c(c3)[C@]3(CCc5ccc(N(c6ccccc6)c6ccccc6)cc53)CC4)c3cccc4ccccc34)cc21 ; CC1(C)c2ccccc2-c2ccc(N(c3ccccc3)c3ccc4c(c3)[C@@]3(CC4)CCc4ccc(N(c5ccccc5)c5ccc6ccccc6c5)cc43)cc21 ; CC1(C)c2ccccc2-c2ccc(N(c3ccccc3)c3ccc4c(c3)[C@@]3(CC4)CCc4ccc(N(c5ccccc5)c5cccc6ccccc56)cc43)cc21 ; CC1(C)c2ccccc2-c2ccc(N(c3ccc(-c4ccccc4)cc3)c3ccc4c(c3)[C@]3(CCc5ccc(N(c6ccccc6)c6ccccc6)cc53)CC4)cc21]

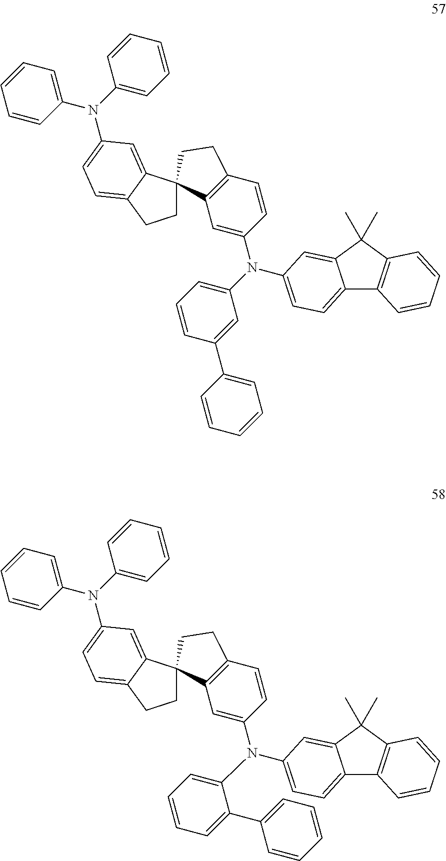 [SMILES: CC1(C)c2ccccc2-c2ccc(N(c3cccc(-c4ccccc4)c3)c3ccc4c(c3)[C@]3(CCc5ccc(N(c6ccccc6)c6ccccc6)cc53)CC4)cc21 ; CC1(C)c2ccccc2-c2ccc(N(c3ccc4c(c3)[C@]3(CCc5ccc(N(c6ccccc6)c6ccccc6)cc53)CC4)c3ccccc3-c3ccccc3)cc21]

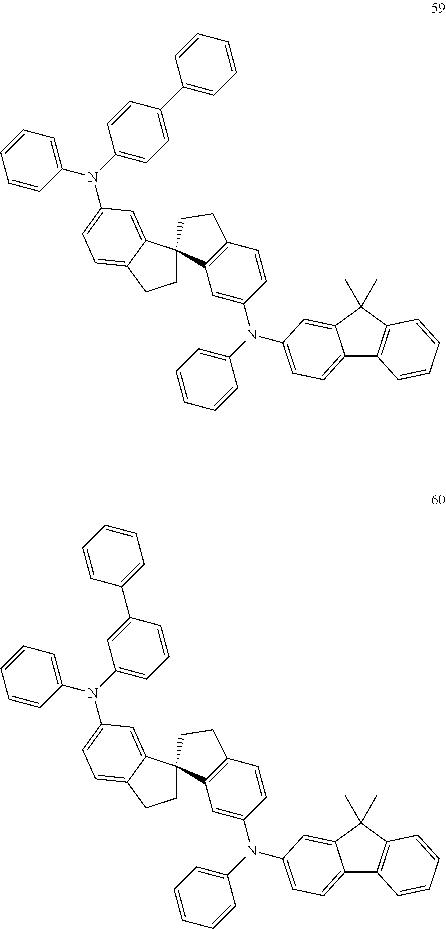 [SMILES: CC1(C)c2ccccc2-c2ccc(N(c3ccccc3)c3ccc4c(c3)[C@]3(CCc5ccc(N(c6ccccc6)c6ccc(-c7ccccc7)cc6)cc53)CC4)cc21 ; CC1(C)c2ccccc2-c2ccc(N(c3ccccc3)c3ccc4c(c3)[C@]3(CCc5ccc(N(c6ccccc6)c6cccc(-c7ccccc7)c6)cc53)CC4)cc21]

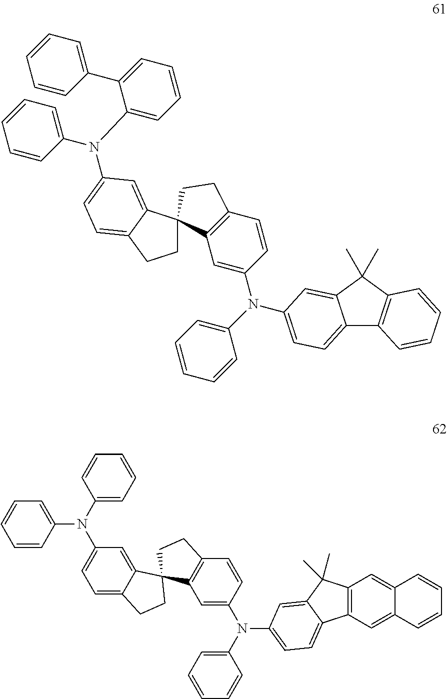 [SMILES: CC1(C)c2ccccc2-c2ccc(N(c3ccccc3)c3ccc4c(c3)[C@@]3(CC4)CCc4ccc(N(c5ccccc5)c5ccccc5-c5ccccc5)cc43)cc21 ; CC1(C)c2cc(N(c3ccccc3)c3ccc4c(c3)[C@]3(CCc5ccc(N(c6ccccc6)c6ccccc6)cc53)CC4)ccc2-c2cc3ccccc3cc21]

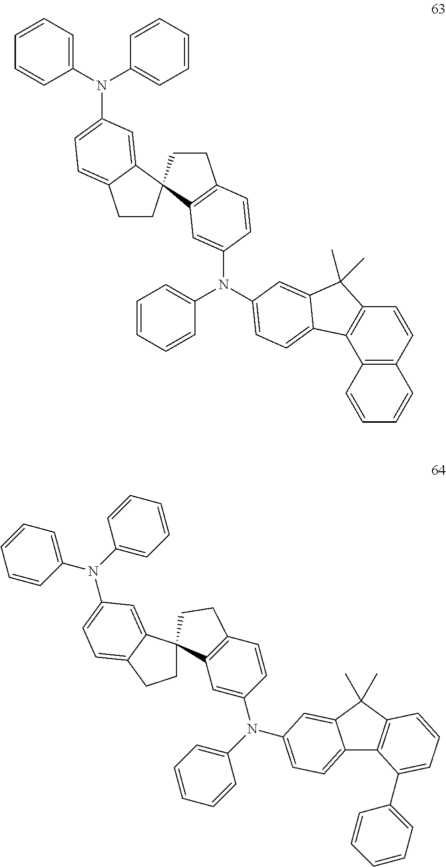 [SMILES: CC1(C)c2cc(N(c3ccccc3)c3ccc4c(c3)[C@]3(CCc5ccc(N(c6ccccc6)c6ccccc6)cc53)CC4)ccc2-c2c1ccc1ccccc21 ; CC1(C)c2cc(N(c3ccccc3)c3ccc4c(c3)[C@]3(CCc5ccc(N(c6ccccc6)c6ccccc6)cc53)CC4)ccc2-c2c(-c3ccccc3)cccc21]

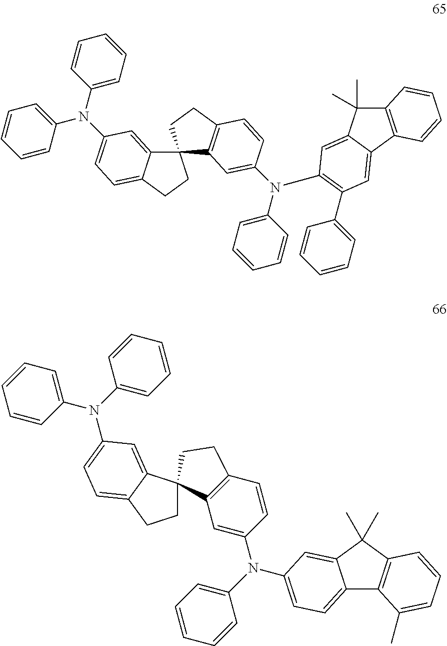 [SMILES: CC1(C)c2ccccc2-c2cc(-c3ccccc3)c(N(c3ccccc3)c3ccc4c(c3)[C@]3(CCc5ccc(N(c6ccccc6)c6ccccc6)cc53)CC4)cc21 ; Cc1cccc2c1-c1ccc(N(c3ccccc3)c3ccc4c(c3)[C@]3(CCc5ccc(N(c6ccccc6)c6ccccc6)cc53)CC4)cc1C2(C)C]

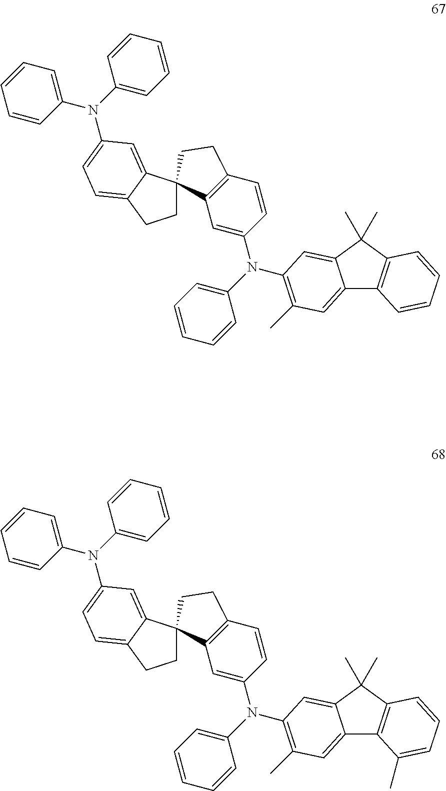 [SMILES: Cc1cc2c(cc1N(c1ccccc1)c1ccc3c(c1)[C@]1(CCc4ccc(N(c5ccccc5)c5ccccc5)cc41)CC3)C(C)(C)c1ccccc1-2 ; Cc1cc2c(cc1N(c1ccccc1)c1ccc3c(c1)[C@]1(CCc4ccc(N(c5ccccc5)c5ccccc5)cc41)CC3)C(C)(C)c1cccc(C)c1-2]

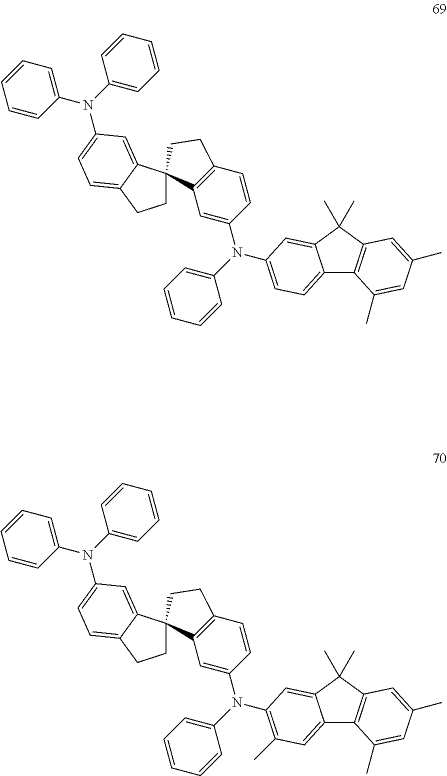 [SMILES: Cc1cc(C)c2c(c1)C(C)(C)c1cc(N(c3ccccc3)c3ccc4c(c3)[C@]3(CCc5ccc(N(c6ccccc6)c6ccccc6)cc53)CC4)ccc1-2 ; Cc1cc(C)c2c(c1)C(C)(C)c1cc(N(c3ccccc3)c3ccc4c(c3)[C@]3(CCc5ccc(N(c6ccccc6)c6ccccc6)cc53)CC4)c(C)cc1-2]

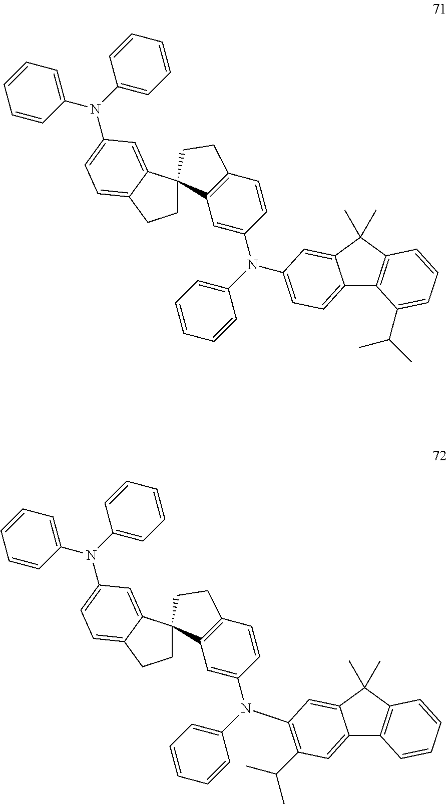 [SMILES: CC(C)c1cccc2c1-c1ccc(N(c3ccccc3)c3ccc4c(c3)[C@]3(CCc5ccc(N(c6ccccc6)c6ccccc6)cc53)CC4)cc1C2(C)C ; CC(C)c1cc2c(cc1N(c1ccccc1)c1ccc3c(c1)[C@]1(CCc4ccc(N(c5ccccc5)c5ccccc5)cc41)CC3)C(C)(C)c1ccccc1-2]

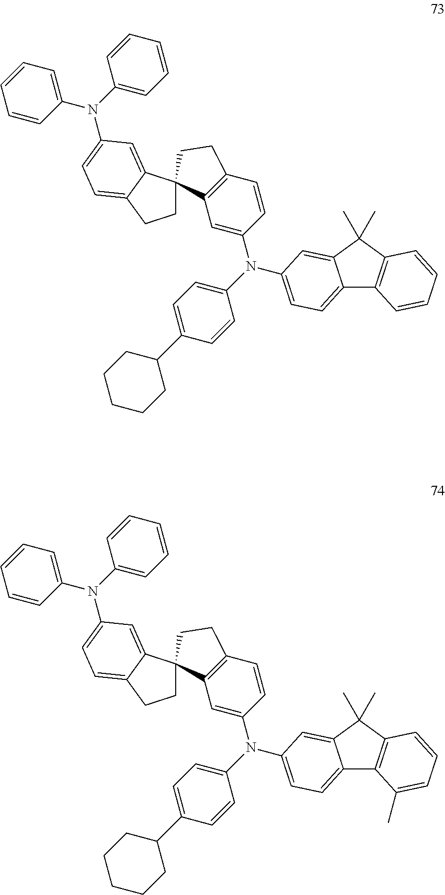 [SMILES: CC1(C)c2ccccc2-c2ccc(N(c3ccc(C4CCCCC4)cc3)c3ccc4c(c3)[C@]3(CCc5ccc(N(c6ccccc6)c6ccccc6)cc53)CC4)cc21 ; Cc1cccc2c1-c1ccc(N(c3ccc(C4CCCCC4)cc3)c3ccc4c(c3)[C@]3(CCc5ccc(N(c6ccccc6)c6ccccc6)cc53)CC4)cc1C2(C)C]

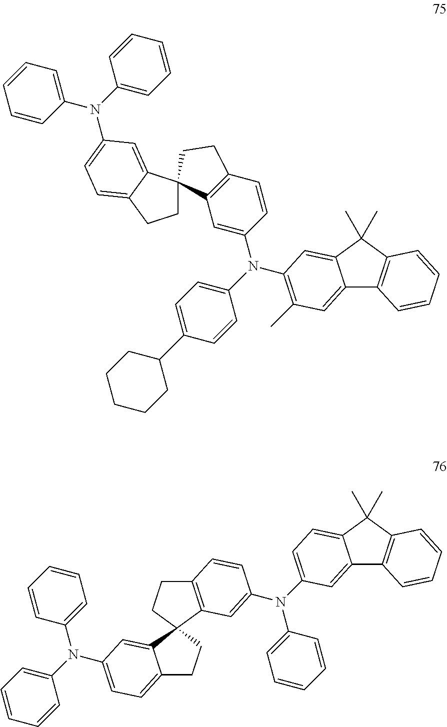 [SMILES: Cc1cc2c(cc1N(c1ccc(C3CCCCC3)cc1)c1ccc3c(c1)[C@]1(CCc4ccc(N(c5ccccc5)c5ccccc5)cc41)CC3)C(C)(C)c1ccccc1-2 ; CC1(C)c2ccccc2-c2cc(N(c3ccccc3)c3ccc4c(c3)[C@]3(CCc5ccc(N(c6ccccc6)c6ccccc6)cc53)CC4)ccc21]

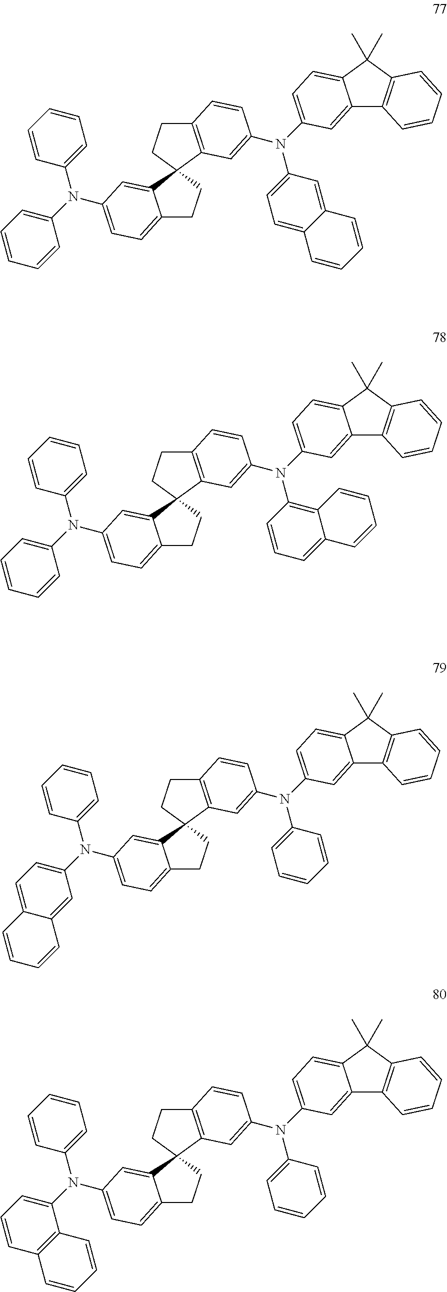 [SMILES: CC1(C)c2ccccc2-c2cc(N(c3ccc4c(c3)[C@]3(CCc5ccc(N(c6ccccc6)c6ccccc6)cc53)CC4)c3ccc4ccccc4c3)ccc21 ; CC1(C)c2ccccc2-c2cc(N(c3ccc4c(c3)[C@]3(CCc5ccc(N(c6ccccc6)c6ccccc6)cc53)CC4)c3cccc4ccccc34)ccc21 ; CC1(C)c2ccccc2-c2cc(N(c3ccccc3)c3ccc4c(c3)[C@@]3(CC4)CCc4ccc(N(c5ccccc5)c5ccc6ccccc6c5)cc43)ccc21 ; CC1(C)c2ccccc2-c2cc(N(c3ccccc3)c3ccc4c(c3)[C@@]3(CC4)CCc4ccc(N(c5ccccc5)c5cccc6ccccc56)cc43)ccc21]

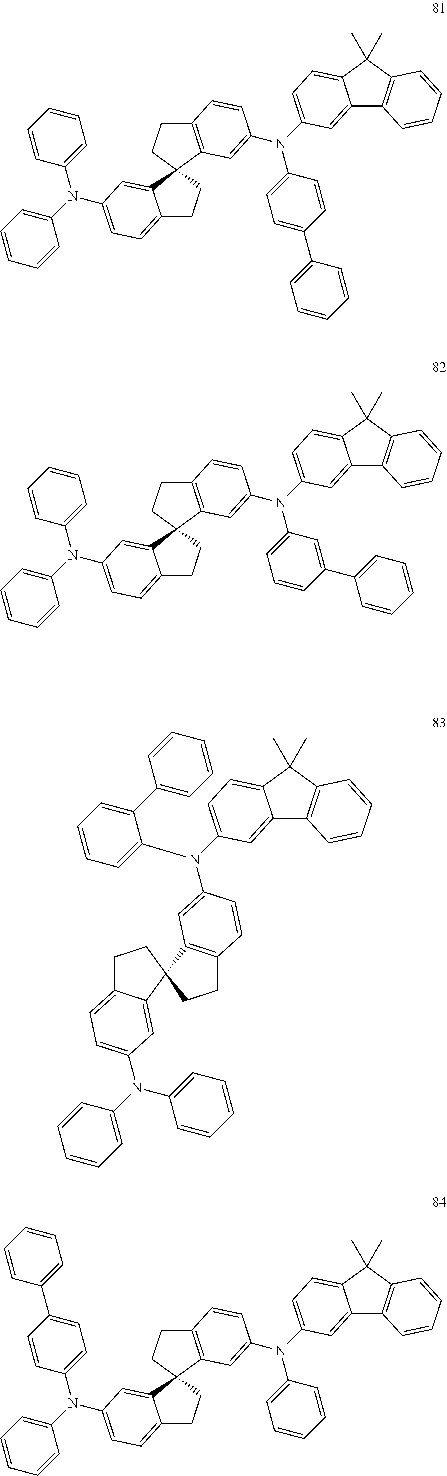 [SMILES: CC1(C)c2ccccc2-c2cc(N(c3ccc(-c4ccccc4)cc3)c3ccc4c(c3)[C@]3(CCc5ccc(N(c6ccccc6)c6ccccc6)cc53)CC4)ccc21 ; CC1(C)c2ccccc2-c2cc(N(c3cccc(-c4ccccc4)c3)c3ccc4c(c3)[C@]3(CCc5ccc(N(c6ccccc6)c6ccccc6)cc53)CC4)ccc21 ; CC1(C)c2ccccc2-c2cc(N(c3ccc4c(c3)[C@]3(CCc5ccc(N(c6ccccc6)c6ccccc6)cc53)CC4)c3ccccc3-c3ccccc3)ccc21 ; CC1(C)c2ccccc2-c2cc(N(c3ccccc3)c3ccc4c(c3)[C@]3(CCc5ccc(N(c6ccccc6)c6ccc(-c7ccccc7)cc6)cc53)CC4)ccc21]

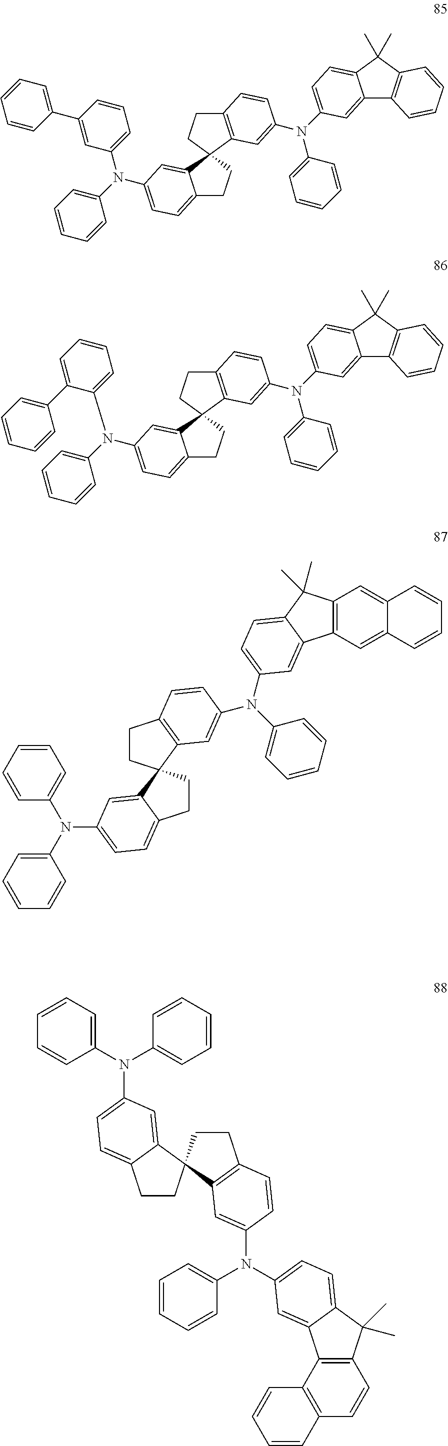 [SMILES: CC1(C)c2ccccc2-c2cc(N(c3ccccc3)c3ccc4c(c3)[C@]3(CCc5ccc(N(c6ccccc6)c6cccc(-c7ccccc7)c6)cc53)CC4)ccc21 ; CC1(C)c2ccccc2-c2cc(N(c3ccccc3)c3ccc4c(c3)[C@@]3(CC4)CCc4ccc(N(c5ccccc5)c5ccccc5-c5ccccc5)cc43)ccc21 ; CC1(C)c2ccc(N(c3ccccc3)c3ccc4c(c3)[C@]3(CCc5ccc(N(c6ccccc6)c6ccccc6)cc53)CC4)cc2-c2cc3ccccc3cc21 ; CC1(C)c2ccc(N(c3ccccc3)c3ccc4c(c3)[C@]3(CCc5ccc(N(c6ccccc6)c6ccccc6)cc53)CC4)cc2-c2c1ccc1ccccc21]

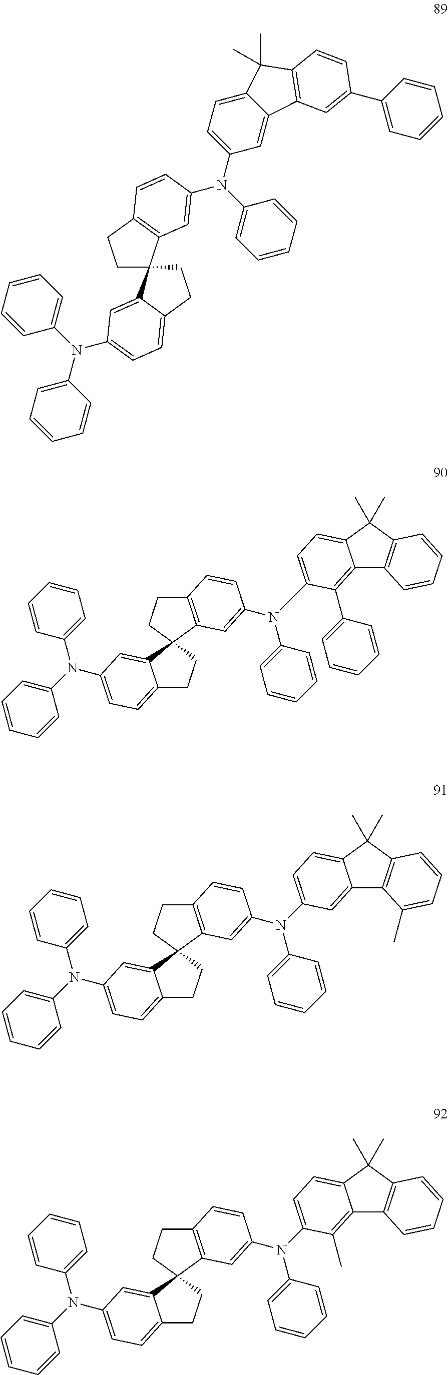 [SMILES: CC1(C)c2ccc(-c3ccccc3)cc2-c2cc(N(c3ccccc3)c3ccc4c(c3)[C@]3(CCc5ccc(N(c6ccccc6)c6ccccc6)cc53)CC4)ccc21 ; CC1(C)c2ccccc2-c2c1ccc(N(c1ccccc1)c1ccc3c(c1)[C@]1(CCc4ccc(N(c5ccccc5)c5ccccc5)cc41)CC3)c2-c1ccccc1 ; Cc1cccc2c1-c1cc(N(c3ccccc3)c3ccc4c(c3)[C@]3(CCc5ccc(N(c6ccccc6)c6ccccc6)cc53)CC4)ccc1C2(C)C ; Cc1c(N(c2ccccc2)c2ccc3c(c2)[C@]2(CCc4ccc(N(c5ccccc5)c5ccccc5)cc42)CC3)ccc2c1-c1ccccc1C2(C)C]

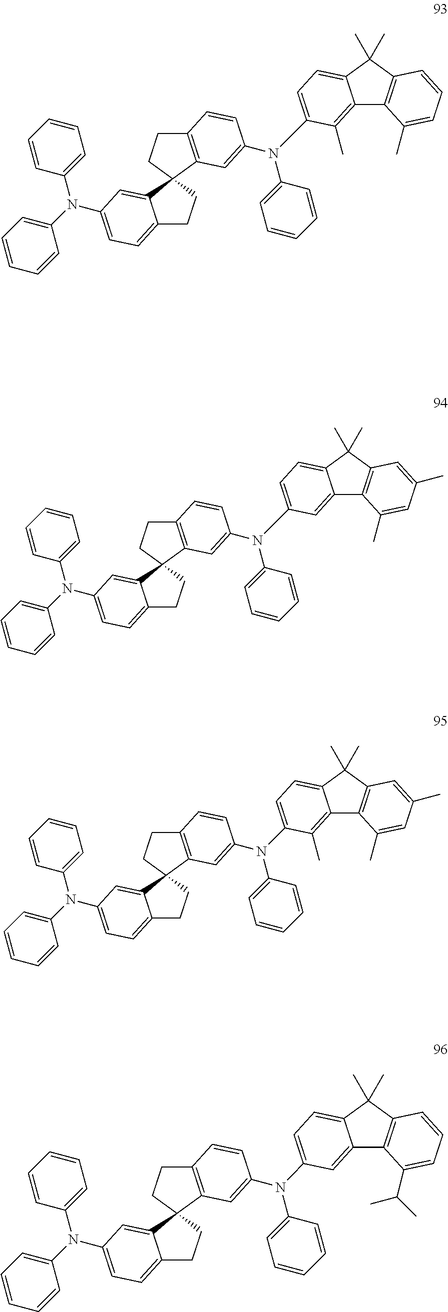 [SMILES: Cc1cccc2c1-c1c(ccc(N(c3ccccc3)c3ccc4c(c3)[C@]3(CCc5ccc(N(c6ccccc6)c6ccccc6)cc53)CC4)c1C)C2(C)C ; Cc1cc(C)c2c(c1)C(C)(C)c1ccc(N(c3ccccc3)c3ccc4c(c3)[C@]3(CCc5ccc(N(c6ccccc6)c6ccccc6)cc53)CC4)cc1-2 ; Cc1cc(C)c2c(c1)C(C)(C)c1ccc(N(c3ccccc3)c3ccc4c(c3)[C@]3(CCc5ccc(N(c6ccccc6)c6ccccc6)cc53)CC4)c(C)c1-2 ; CC(C)c1cccc2c1-c1cc(N(c3ccccc3)c3ccc4c(c3)[C@]3(CCc5ccc(N(c6ccccc6)c6ccccc6)cc53)CC4)ccc1C2(C)C]

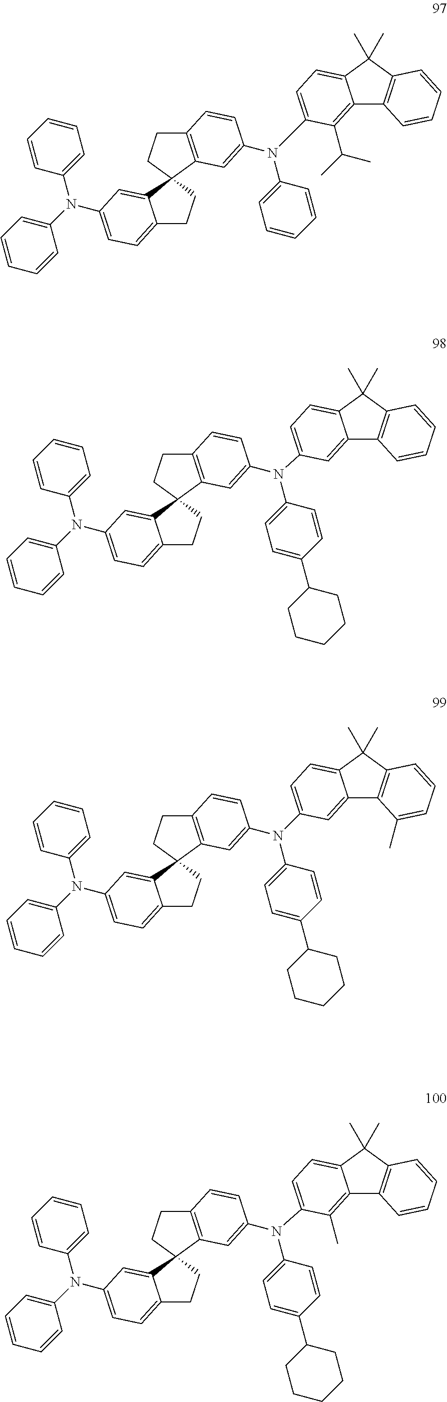 [SMILES: CC(C)c1c(N(c2ccccc2)c2ccc3c(c2)[C@]2(CCc4ccc(N(c5ccccc5)c5ccccc5)cc42)CC3)ccc2c1-c1ccccc1C2(C)C ; CC1(C)c2ccccc2-c2cc(N(c3ccc(C4CCCCC4)cc3)c3ccc4c(c3)[C@]3(CCc5ccc(N(c6ccccc6)c6ccccc6)cc53)CC4)ccc21 ; Cc1cccc2c1-c1cc(N(c3ccc(C4CCCCC4)cc3)c3ccc4c(c3)[C@]3(CCc5ccc(N(c6ccccc6)c6ccccc6)cc53)CC4)ccc1C2(C)C ; Cc1c(N(c2ccc(C3CCCCC3)cc2)c2ccc3c(c2)[C@]2(CCc4ccc(N(c5ccccc5)c5ccccc5)cc42)CC3)ccc2c1-c1ccccc1C2(C)C]

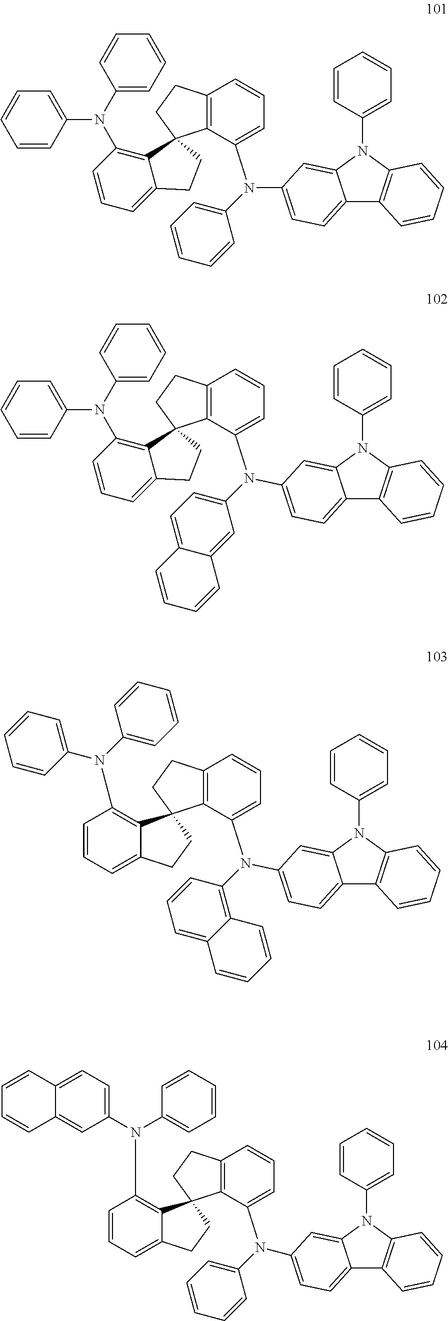 [SMILES: c1ccc(N(c2ccccc2)c2cccc3c2[C@@]2(CC3)CCc3cccc(N(c4ccccc4)c4ccc5c6ccccc6n(-c6ccccc6)c5c4)c32)cc1 ; c1ccc(N(c2ccccc2)c2cccc3c2[C@@]2(CC3)CCc3cccc(N(c4ccc5ccccc5c4)c4ccc5c6ccccc6n(-c6ccccc6)c5c4)c32)cc1 ; c1ccc(N(c2ccccc2)c2cccc3c2[C@@]2(CC3)CCc3cccc(N(c4ccc5c6ccccc6n(-c6ccccc6)c5c4)c4cccc5ccccc45)c32)cc1 ; c1ccc(N(c2ccc3ccccc3c2)c2cccc3c2[C@@]2(CC3)CCc3cccc(N(c4ccccc4)c4ccc5c6ccccc6n(-c6ccccc6)c5c4)c32)cc1]

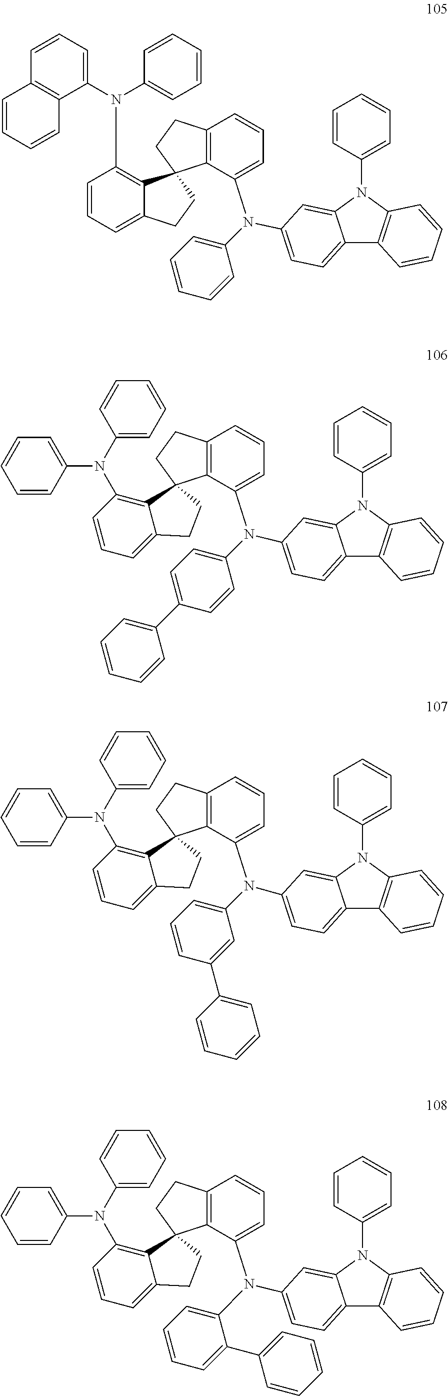 [SMILES: c1ccc(N(c2ccc3c4ccccc4n(-c4ccccc4)c3c2)c2cccc3c2[C@@]2(CC3)CCc3cccc(N(c4ccccc4)c4cccc5ccccc45)c32)cc1 ; c1ccc(-c2ccc(N(c3ccc4c5ccccc5n(-c5ccccc5)c4c3)c3cccc4c3[C@]3(CCc5cccc(N(c6ccccc6)c6ccccc6)c53)CC4)cc2)cc1 ; c1ccc(-c2cccc(N(c3ccc4c5ccccc5n(-c5ccccc5)c4c3)c3cccc4c3[C@]3(CCc5cccc(N(c6ccccc6)c6ccccc6)c53)CC4)c2)cc1 ; c1ccc(-c2ccccc2N(c2ccc3c4ccccc4n(-c4ccccc4)c3c2)c2cccc3c2[C@]2(CCc4cccc(N(c5ccccc5)c5ccccc5)c42)CC3)cc1]

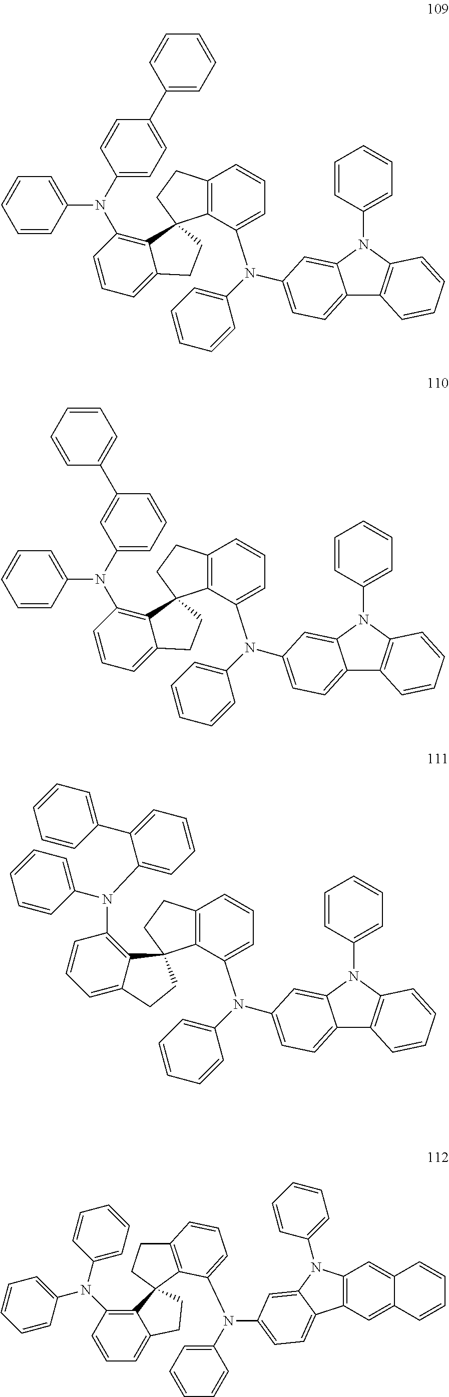 [SMILES: c1ccc(-c2ccc(N(c3ccccc3)c3cccc4c3[C@@]3(CC4)CCc4cccc(N(c5ccccc5)c5ccc6c7ccccc7n(-c7ccccc7)c6c5)c43)cc2)cc1 ; c1ccc(-c2cccc(N(c3ccccc3)c3cccc4c3[C@@]3(CC4)CCc4cccc(N(c5ccccc5)c5ccc6c7ccccc7n(-c7ccccc7)c6c5)c43)c2)cc1 ; c1ccc(-c2ccccc2N(c2ccccc2)c2cccc3c2[C@]2(CCc4cccc(N(c5ccccc5)c5ccc6c7ccccc7n(-c7ccccc7)c6c5)c42)CC3)cc1 ; c1ccc(N(c2ccccc2)c2cccc3c2[C@@]2(CC3)CCc3cccc(N(c4ccccc4)c4ccc5c6cc7ccccc7cc6n(-c6ccccc6)c5c4)c32)cc1]

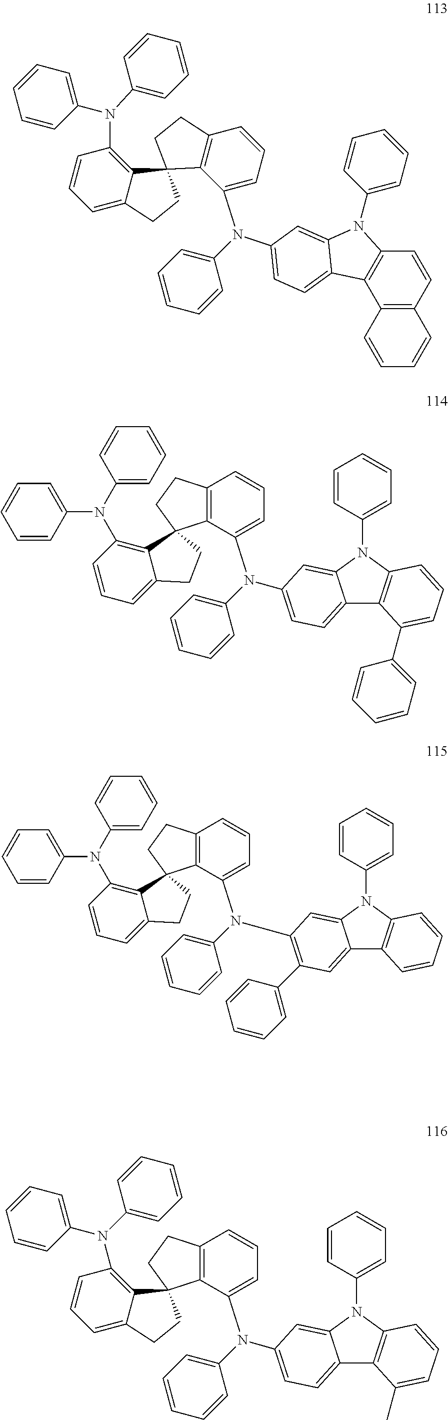 [SMILES: c1ccc(N(c2ccccc2)c2cccc3c2[C@@]2(CC3)CCc3cccc(N(c4ccccc4)c4ccc5c6c7ccccc7ccc6n(-c6ccccc6)c5c4)c32)cc1 ; c1ccc(-c2cccc3c2c2ccc(N(c4ccccc4)c4cccc5c4[C@]4(CCc6cccc(N(c7ccccc7)c7ccccc7)c64)CC5)cc2n3-c2ccccc2)cc1 ; c1ccc(-c2cc3c4ccccc4n(-c4ccccc4)c3cc2N(c2ccccc2)c2cccc3c2[C@]2(CCc4cccc(N(c5ccccc5)c5ccccc5)c42)CC3)cc1 ; Cc1cccc2c1c1ccc(N(c3ccccc3)c3cccc4c3[C@]3(CCc5cccc(N(c6ccccc6)c6ccccc6)c53)CC4)cc1n2-c1ccccc1]

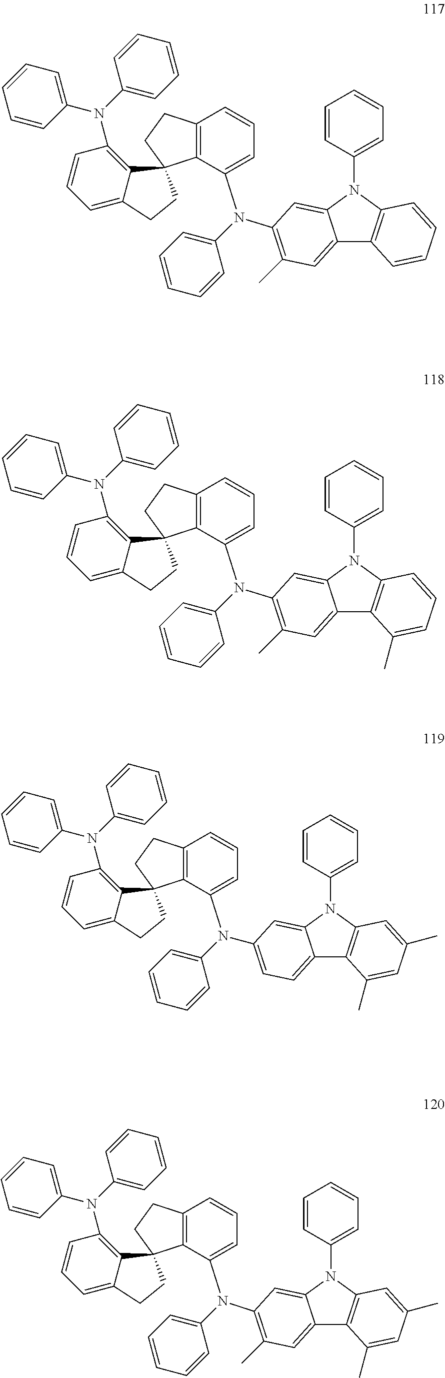 [SMILES: Cc1cc2c3ccccc3n(-c3ccccc3)c2cc1N(c1ccccc1)c1cccc2c1[C@]1(CCc3cccc(N(c4ccccc4)c4ccccc4)c31)CC2 ; Cc1cc2c3c(C)cccc3n(-c3ccccc3)c2cc1N(c1ccccc1)c1cccc2c1[C@]1(CCc3cccc(N(c4ccccc4)c4ccccc4)c31)CC2 ; Cc1cc(C)c2c3ccc(N(c4ccccc4)c4cccc5c4[C@]4(CCc6cccc(N(c7ccccc7)c7ccccc7)c64)CC5)cc3n(-c3ccccc3)c2c1 ; Cc1cc(C)c2c3cc(C)c(N(c4ccccc4)c4cccc5c4[C@]4(CCc6cccc(N(c7ccccc7)c7ccccc7)c64)CC5)cc3n(-c3ccccc3)c2c1]

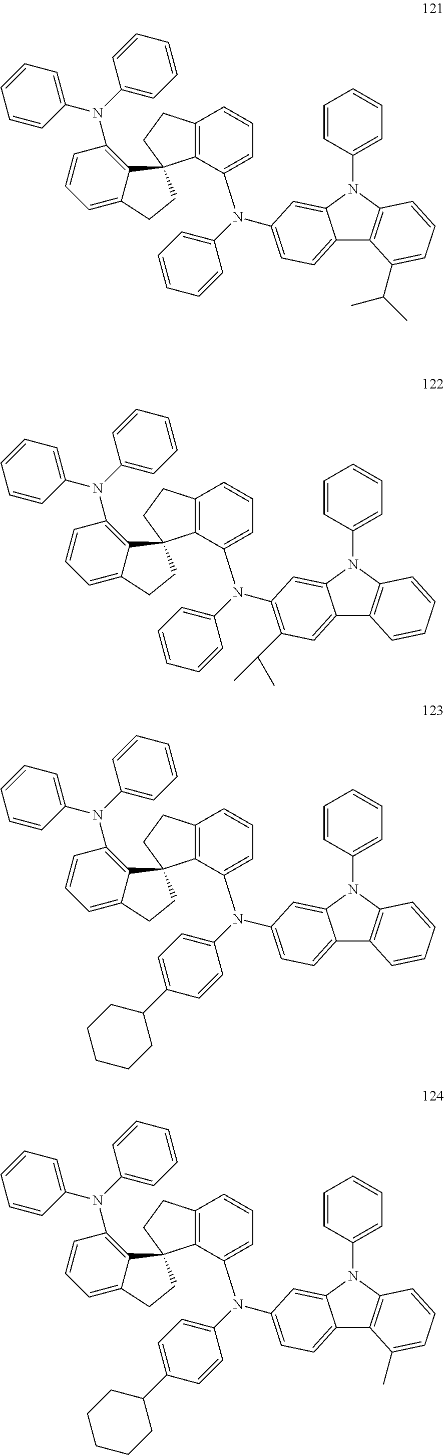 [SMILES: CC(C)c1cccc2c1c1ccc(N(c3ccccc3)c3cccc4c3[C@]3(CCc5cccc(N(c6ccccc6)c6ccccc6)c53)CC4)cc1n2-c1ccccc1 ; CC(C)c1cc2c3ccccc3n(-c3ccccc3)c2cc1N(c1ccccc1)c1cccc2c1[C@]1(CCc3cccc(N(c4ccccc4)c4ccccc4)c31)CC2 ; c1ccc(N(c2ccccc2)c2cccc3c2[C@@]2(CC3)CCc3cccc(N(c4ccc(C5CCCCC5)cc4)c4ccc5c6ccccc6n(-c6ccccc6)c5c4)c32)cc1 ; Cc1cccc2c1c1ccc(N(c3ccc(C4CCCCC4)cc3)c3cccc4c3[C@]3(CCc5cccc(N(c6ccccc6)c6ccccc6)c53)CC4)cc1n2-c1ccccc1]

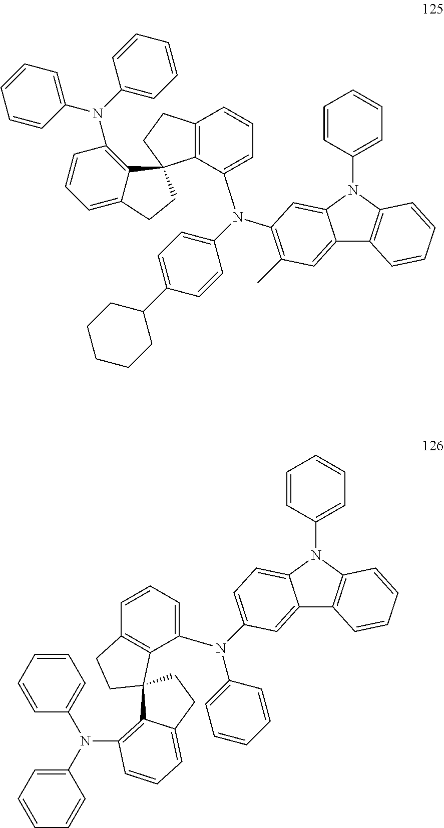 [SMILES: Cc1cc2c3ccccc3n(-c3ccccc3)c2cc1N(c1ccc(C2CCCCC2)cc1)c1cccc2c1[C@]1(CCc3cccc(N(c4ccccc4)c4ccccc4)c31)CC2 ; c1ccc(N(c2ccccc2)c2cccc3c2[C@@]2(CC3)CCc3cccc(N(c4ccccc4)c4ccc5c(c4)c4ccccc4n5-c4ccccc4)c32)cc1]

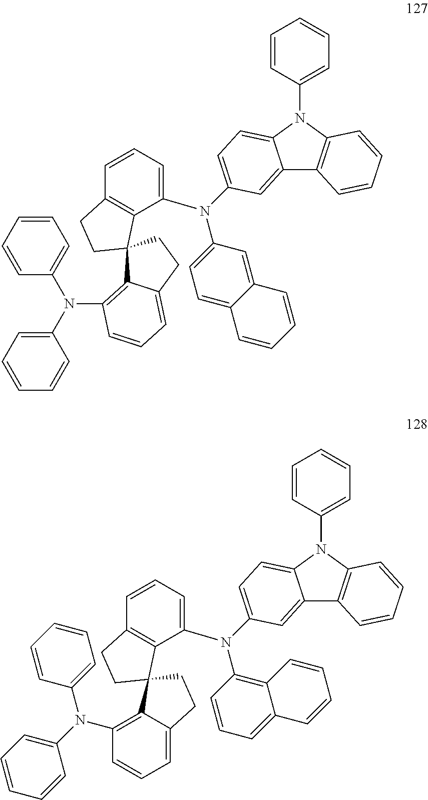 [SMILES: c1ccc(N(c2ccccc2)c2cccc3c2[C@@]2(CC3)CCc3cccc(N(c4ccc5ccccc5c4)c4ccc5c(c4)c4ccccc4n5-c4ccccc4)c32)cc1 ; c1ccc(N(c2ccccc2)c2cccc3c2[C@@]2(CC3)CCc3cccc(N(c4ccc5c(c4)c4ccccc4n5-c4ccccc4)c4cccc5ccccc45)c32)cc1]

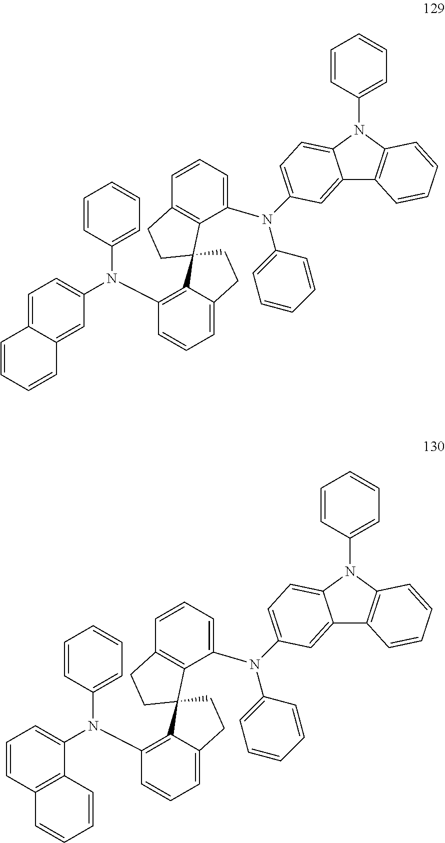 [SMILES: c1ccc(N(c2ccc3ccccc3c2)c2cccc3c2[C@@]2(CC3)CCc3cccc(N(c4ccccc4)c4ccc5c(c4)c4ccccc4n5-c4ccccc4)c32)cc1 ; c1ccc(N(c2ccc3c(c2)c2ccccc2n3-c2ccccc2)c2cccc3c2[C@@]2(CC3)CCc3cccc(N(c4ccccc4)c4cccc5ccccc45)c32)cc1]

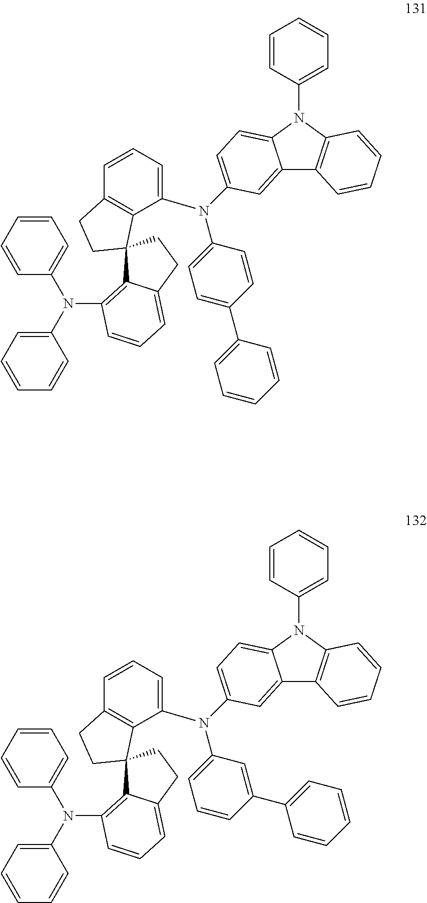 [SMILES: c1ccc(-c2ccc(N(c3ccc4c(c3)c3ccccc3n4-c3ccccc3)c3cccc4c3[C@]3(CCc5cccc(N(c6ccccc6)c6ccccc6)c53)CC4)cc2)cc1 ; c1ccc(-c2cccc(N(c3ccc4c(c3)c3ccccc3n4-c3ccccc3)c3cccc4c3[C@]3(CCc5cccc(N(c6ccccc6)c6ccccc6)c53)CC4)c2)cc1]

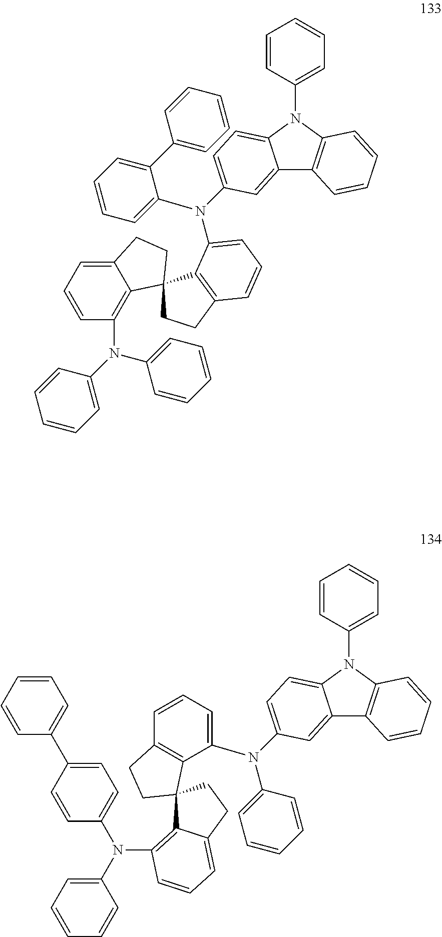 [SMILES: c1ccc(-c2ccccc2N(c2ccc3c(c2)c2ccccc2n3-c2ccccc2)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6c(c5)c5ccccc5n6-c5ccccc5)c43)cc2)cc1]

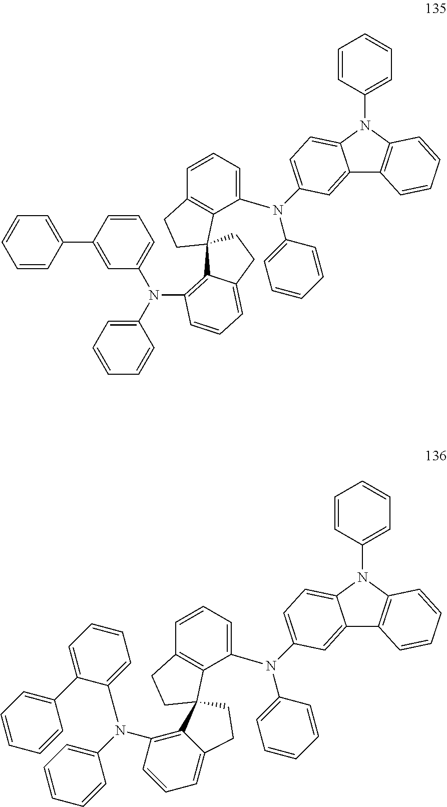 [SMILES: c1ccc(-c2cccc(N(c3ccccc3)c3cccc4c3[C@@]3(CC4)CCc4cccc(N(c5ccccc5)c5ccc6c(c5)c5ccccc5n6-c5ccccc5)c43)c2)cc1 ; c1ccc(-c2ccccc2N(c2ccccc2)c2cccc3c2[C@]2(CCc4cccc(N(c5ccccc5)c5ccc6c(c5)c5ccccc5n6-c5ccccc5)c42)CC3)cc1]

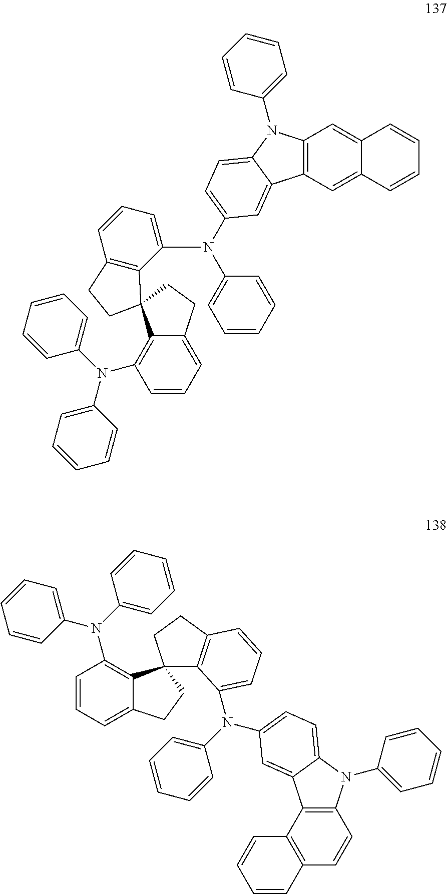 [SMILES: c1ccc(N(c2ccccc2)c2cccc3c2[C@@]2(CC3)CCc3cccc(N(c4ccccc4)c4ccc5c(c4)c4cc6ccccc6cc4n5-c4ccccc4)c32)cc1 ; c1ccc(N(c2ccccc2)c2cccc3c2[C@@]2(CC3)CCc3cccc(N(c4ccccc4)c4ccc5c(c4)c4c6ccccc6ccc4n5-c4ccccc4)c32)cc1]

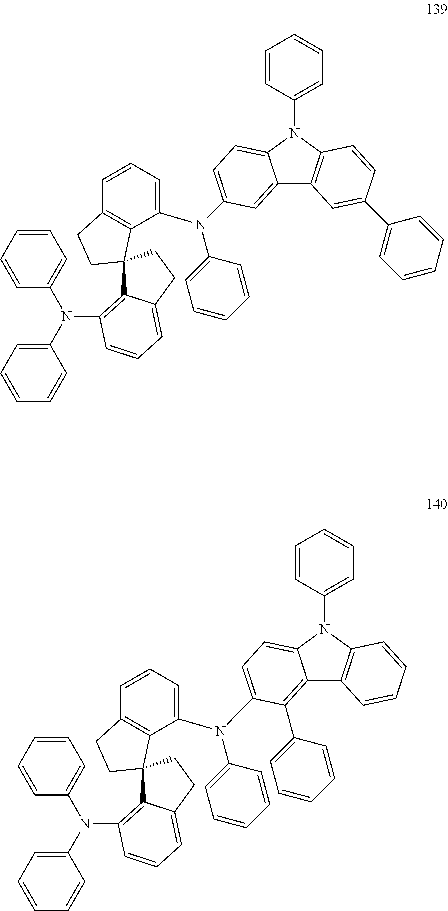 [SMILES: c1ccc(-c2ccc3c(c2)c2cc(N(c4ccccc4)c4cccc5c4[C@]4(CCc6cccc(N(c7ccccc7)c7ccccc7)c64)CC5)ccc2n3-c2ccccc2)cc1 ; c1ccc(-c2c(N(c3ccccc3)c3cccc4c3[C@]3(CCc5cccc(N(c6ccccc6)c6ccccc6)c53)CC4)ccc3c2c2ccccc2n3-c2ccccc2)cc1]

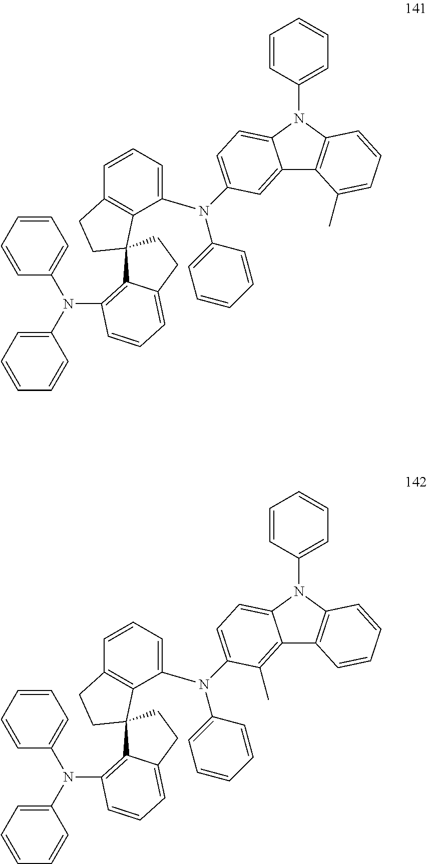 [SMILES: Cc1cccc2c1c1cc(N(c3ccccc3)c3cccc4c3[C@]3(CCc5cccc(N(c6ccccc6)c6ccccc6)c53)CC4)ccc1n2-c1ccccc1 ; Cc1c(N(c2ccccc2)c2cccc3c2[C@]2(CCc4cccc(N(c5ccccc5)c5ccccc5)c42)CC3)ccc2c1c1ccccc1n2-c1ccccc1]

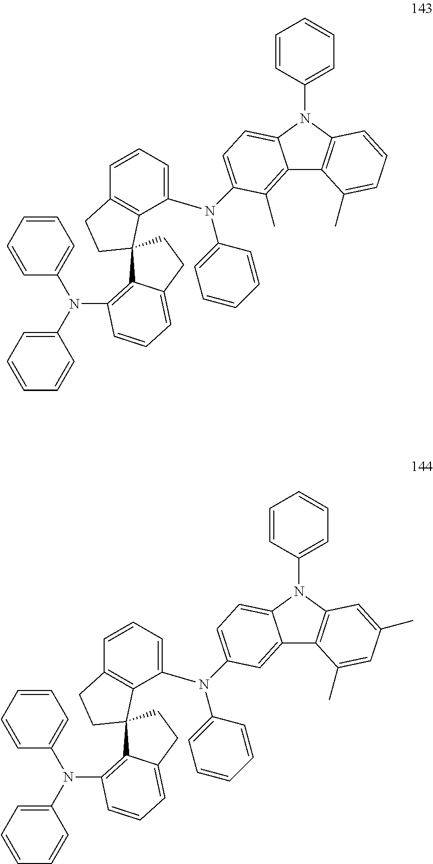 [SMILES: Cc1cccc2c1c1c(C)c(N(c3ccccc3)c3cccc4c3[C@]3(CCc5cccc(N(c6ccccc6)c6ccccc6)c53)CC4)ccc1n2-c1ccccc1 ; Cc1cc(C)c2c3cc(N(c4ccccc4)c4cccc5c4[C@]4(CCc6cccc(N(c7ccccc7)c7ccccc7)c64)CC5)ccc3n(-c3ccccc3)c2c1]

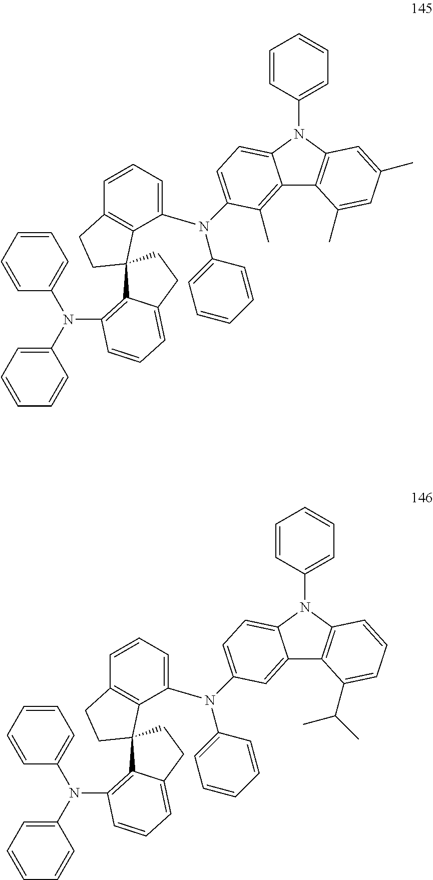 [SMILES: Cc1cc(C)c2c3c(C)c(N(c4ccccc4)c4cccc5c4[C@]4(CCc6cccc(N(c7ccccc7)c7ccccc7)c64)CC5)ccc3n(-c3ccccc3)c2c1 ; CC(C)c1cccc2c1c1cc(N(c3ccccc3)c3cccc4c3[C@]3(CCc5cccc(N(c6ccccc6)c6ccccc6)c53)CC4)ccc1n2-c1ccccc1]

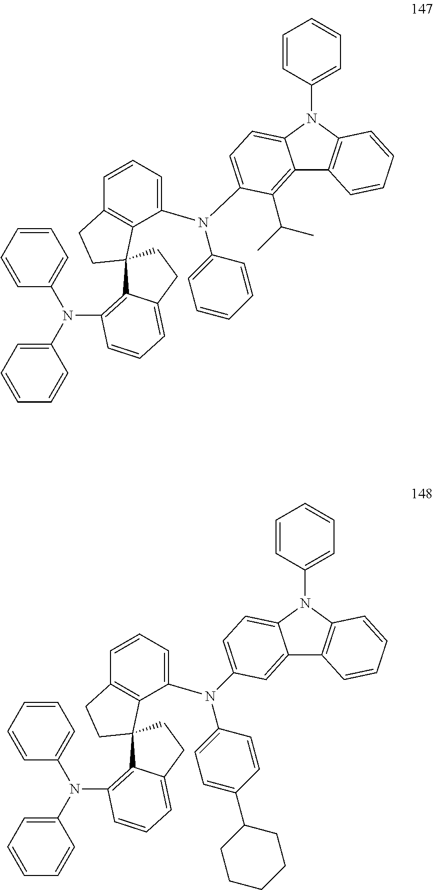 [SMILES: CC(C)c1c(N(c2ccccc2)c2cccc3c2[C@]2(CCc4cccc(N(c5ccccc5)c5ccccc5)c42)CC3)ccc2c1c1ccccc1n2-c1ccccc1 ; c1ccc(N(c2ccccc2)c2cccc3c2[C@@]2(CC3)CCc3cccc(N(c4ccc(C5CCCCC5)cc4)c4ccc5c(c4)c4ccccc4n5-c4ccccc4)c32)cc1]

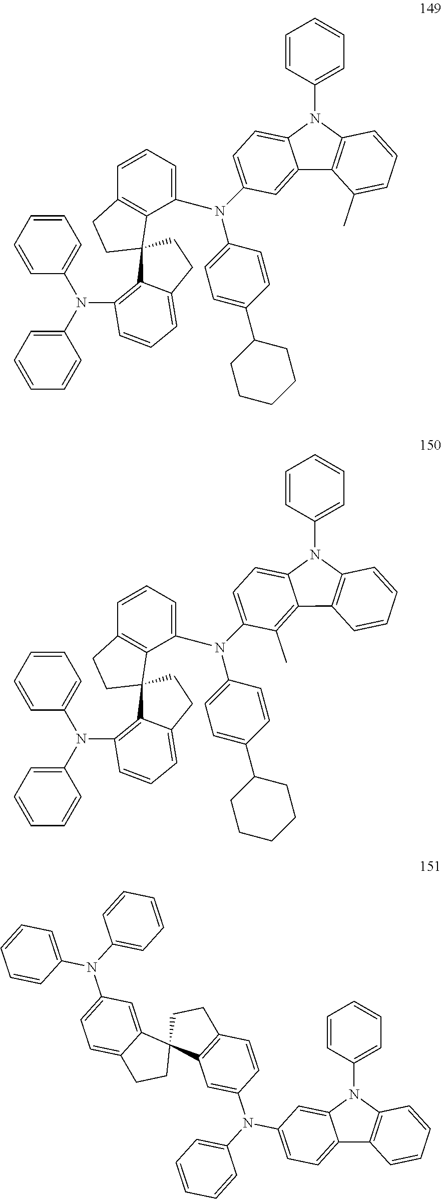 [SMILES: Cc1cccc2c1c1cc(N(c3ccc(C4CCCCC4)cc3)c3cccc4c3[C@]3(CCc5cccc(N(c6ccccc6)c6ccccc6)c53)CC4)ccc1n2-c1ccccc1 ; Cc1c(N(c2ccc(C3CCCCC3)cc2)c2cccc3c2[C@]2(CCc4cccc(N(c5ccccc5)c5ccccc5)c42)CC3)ccc2c1c1ccccc1n2-c1ccccc1 ; c1ccc(N(c2ccccc2)c2ccc3c(c2)[C@@]2(CC3)CCc3ccc(N(c4ccccc4)c4ccc5c6ccccc6n(-c6ccccc6)c5c4)cc32)cc1]

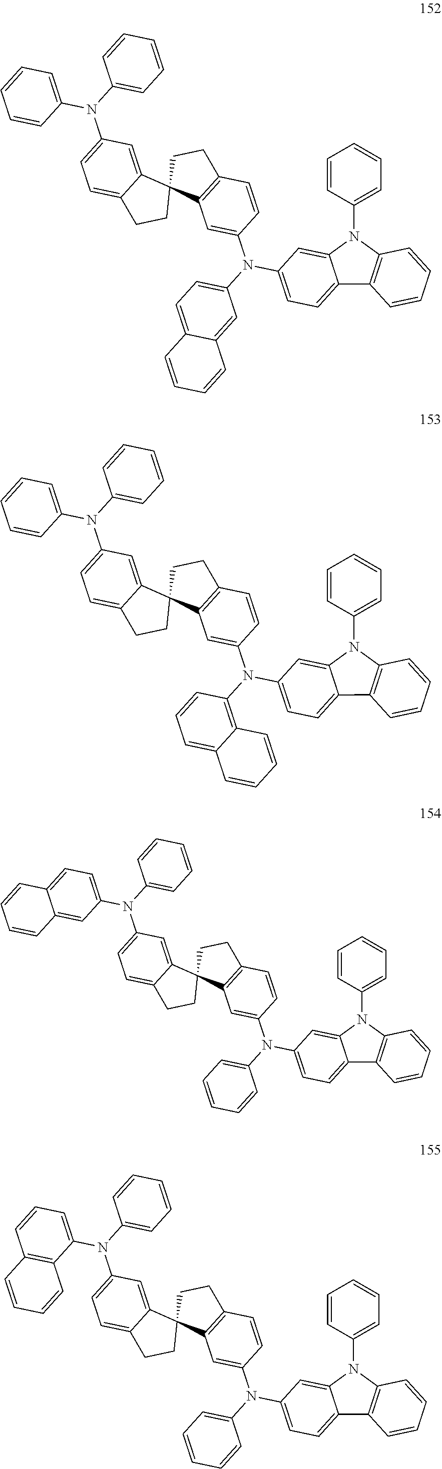 [SMILES: c1ccc(N(c2ccccc2)c2ccc3c(c2)[C@@]2(CC3)CCc3ccc(N(c4ccc5ccccc5c4)c4ccc5c6ccccc6n(-c6ccccc6)c5c4)cc32)cc1 ; c1ccc(N(c2ccccc2)c2ccc3c(c2)[C@@]2(CC3)CCc3ccc(N(c4ccc5c6ccccc6n(-c6ccccc6)c5c4)c4cccc5ccccc45)cc32)cc1 ; c1ccc(N(c2ccc3c(c2)[C@@]2(CC3)CCc3ccc(N(c4ccccc4)c4ccc5c6ccccc6n(-c6ccccc6)c5c4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c4ccccc4n(-c4ccccc4)c3c2)cc1]

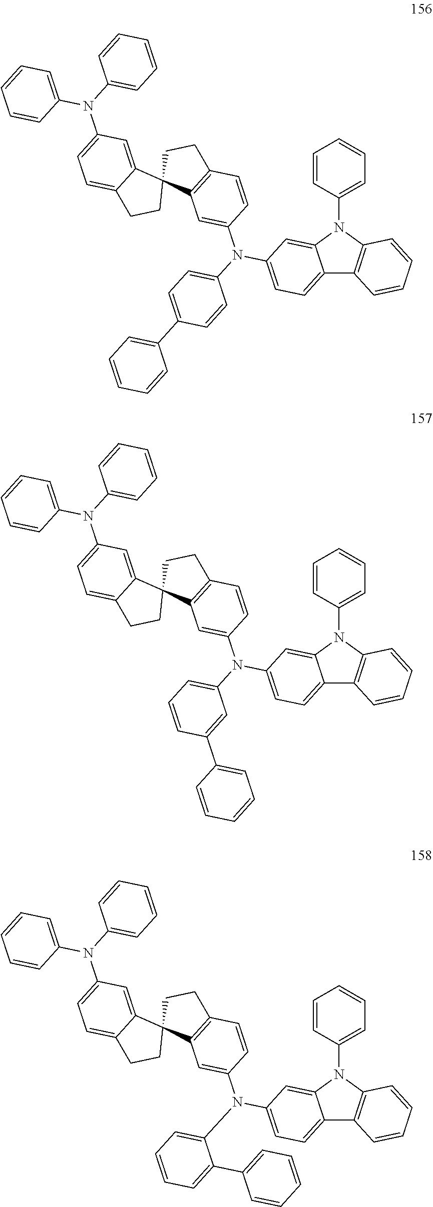 [SMILES: c1ccc(-c2ccc(N(c3ccc4c(c3)[C@]3(CCc5ccc(N(c6ccccc6)c6ccccc6)cc53)CC4)c3ccc4c5ccccc5n(-c5ccccc5)c4c3)cc2)cc1 ; c1ccc(-c2cccc(N(c3ccc4c(c3)[C@]3(CCc5ccc(N(c6ccccc6)c6ccccc6)cc53)CC4)c3ccc4c5ccccc5n(-c5ccccc5)c4c3)c2)cc1 ; c1ccc(-c2ccccc2N(c2ccc3c(c2)[C@]2(CCc4ccc(N(c5ccccc5)c5ccccc5)cc42)CC3)c2ccc3c4ccccc4n(-c4ccccc4)c3c2)cc1]

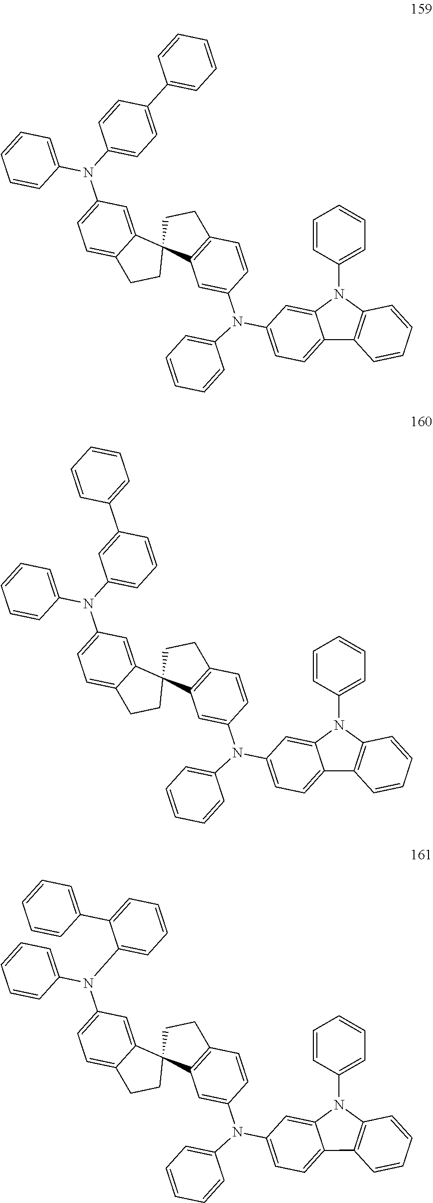 [SMILES: c1ccc(-c2ccc(N(c3ccccc3)c3ccc4c(c3)[C@@]3(CC4)CCc4ccc(N(c5ccccc5)c5ccc6c7ccccc7n(-c7ccccc7)c6c5)cc43)cc2)cc1 ; c1ccc(-c2cccc(N(c3ccccc3)c3ccc4c(c3)[C@@]3(CC4)CCc4ccc(N(c5ccccc5)c5ccc6c7ccccc7n(-c7ccccc7)c6c5)cc43)c2)cc1 ; c1ccc(-c2ccccc2N(c2ccccc2)c2ccc3c(c2)[C@]2(CCc4ccc(N(c5ccccc5)c5ccc6c7ccccc7n(-c7ccccc7)c6c5)cc42)CC3)cc1]

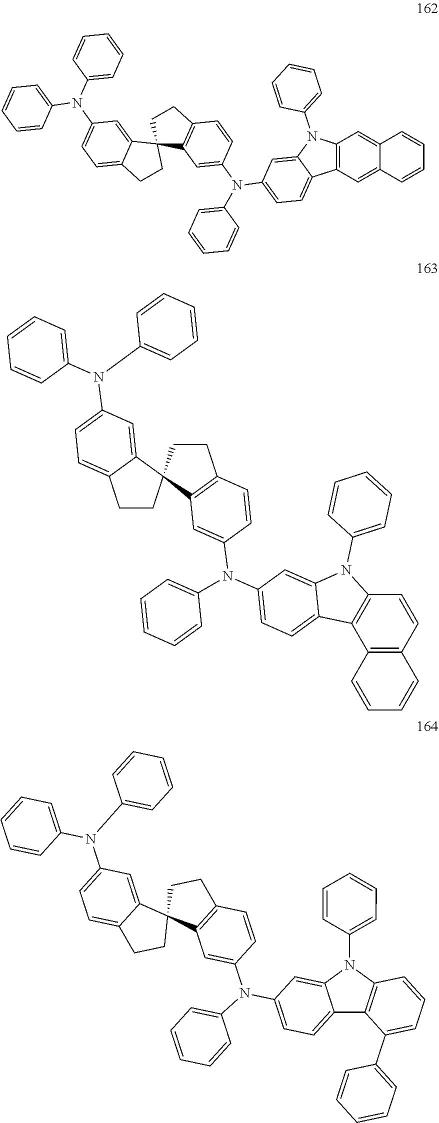 [SMILES: c1ccc(N(c2ccccc2)c2ccc3c(c2)[C@@]2(CC3)CCc3ccc(N(c4ccccc4)c4ccc5c6cc7ccccc7cc6n(-c6ccccc6)c5c4)cc32)cc1 ; c1ccc(N(c2ccccc2)c2ccc3c(c2)[C@@]2(CC3)CCc3ccc(N(c4ccccc4)c4ccc5c6c7ccccc7ccc6n(-c6ccccc6)c5c4)cc32)cc1 ; c1ccc(-c2cccc3c2c2ccc(N(c4ccccc4)c4ccc5c(c4)[C@]4(CCc6ccc(N(c7ccccc7)c7ccccc7)cc64)CC5)cc2n3-c2ccccc2)cc1]

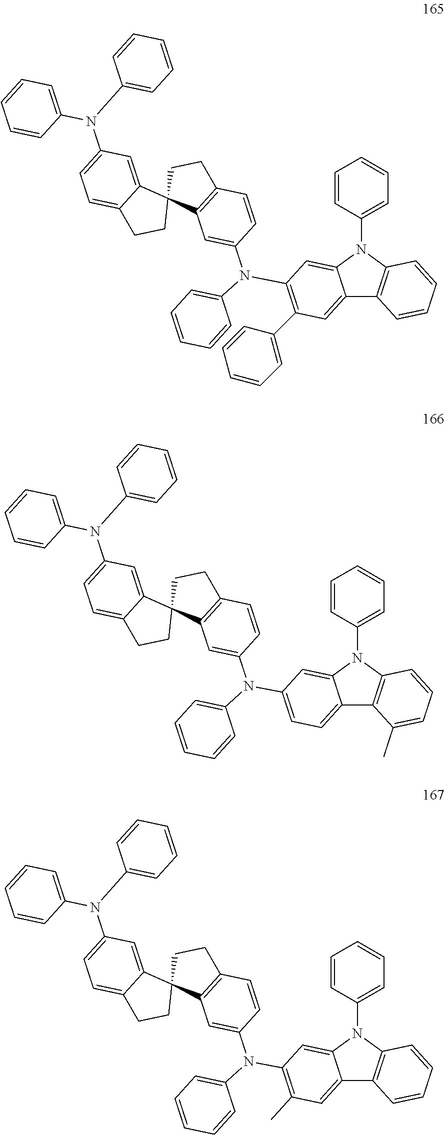 [SMILES: c1ccc(-c2cc3c4ccccc4n(-c4ccccc4)c3cc2N(c2ccccc2)c2ccc3c(c2)[C@]2(CCc4ccc(N(c5ccccc5)c5ccccc5)cc42)CC3)cc1 ; Cc1cccc2c1c1ccc(N(c3ccccc3)c3ccc4c(c3)[C@]3(CCc5ccc(N(c6ccccc6)c6ccccc6)cc53)CC4)cc1n2-c1ccccc1 ; Cc1cc2c3ccccc3n(-c3ccccc3)c2cc1N(c1ccccc1)c1ccc2c(c1)[C@]1(CCc3ccc(N(c4ccccc4)c4ccccc4)cc31)CC2]

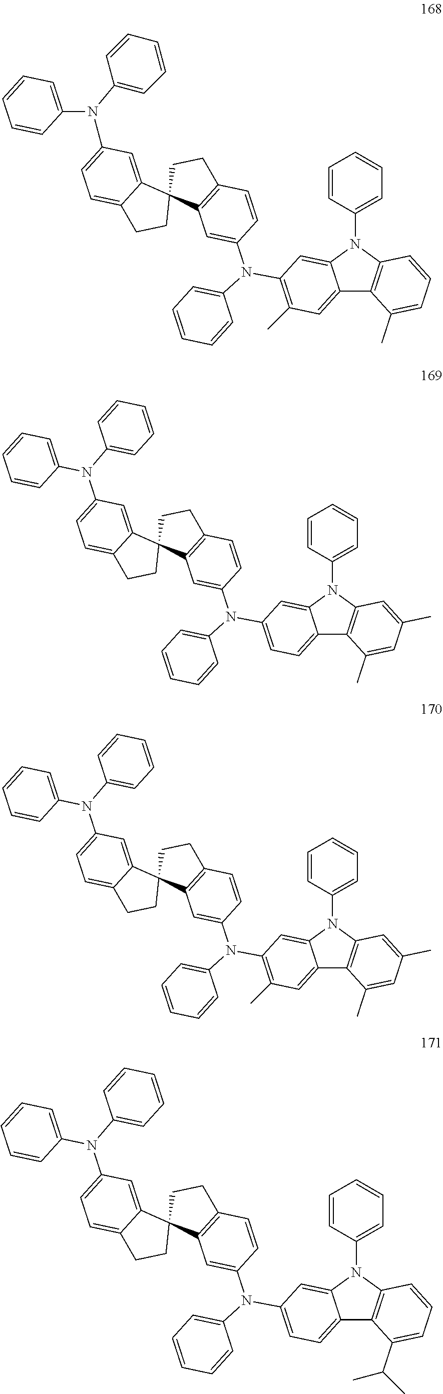 [SMILES: Cc1cc2c3c(C)cccc3n(-c3ccccc3)c2cc1N(c1ccccc1)c1ccc2c(c1)[C@]1(CCc3ccc(N(c4ccccc4)c4ccccc4)cc31)CC2 ; Cc1cc(C)c2c3ccc(N(c4ccccc4)c4ccc5c(c4)[C@]4(CCc6ccc(N(c7ccccc7)c7ccccc7)cc64)CC5)cc3n(-c3ccccc3)c2c1 ; Cc1cc(C)c2c3cc(C)c(N(c4ccccc4)c4ccc5c(c4)[C@]4(CCc6ccc(N(c7ccccc7)c7ccccc7)cc64)CC5)cc3n(-c3ccccc3)c2c1 ; CC(C)c1cccc2c1c1ccc(N(c3ccccc3)c3ccc4c(c3)[C@]3(CCc5ccc(N(c6ccccc6)c6ccccc6)cc53)CC4)cc1n2-c1ccccc1]

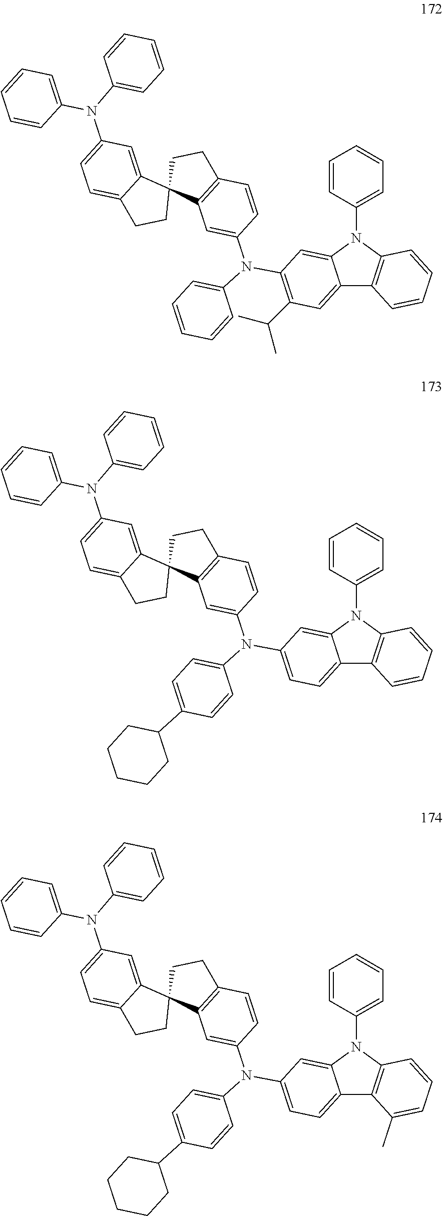 [SMILES: CC(C)c1cc2c3ccccc3n(-c3ccccc3)c2cc1N(c1ccccc1)c1ccc2c(c1)[C@]1(CCc3ccc(N(c4ccccc4)c4ccccc4)cc31)CC2 ; c1ccc(N(c2ccccc2)c2ccc3c(c2)[C@@]2(CC3)CCc3ccc(N(c4ccc(C5CCCCC5)cc4)c4ccc5c6ccccc6n(-c6ccccc6)c5c4)cc32)cc1 ; Cc1cccc2c1c1ccc(N(c3ccc(C4CCCCC4)cc3)c3ccc4c(c3)[C@]3(CCc5ccc(N(c6ccccc6)c6ccccc6)cc53)CC4)cc1n2-c1ccccc1]

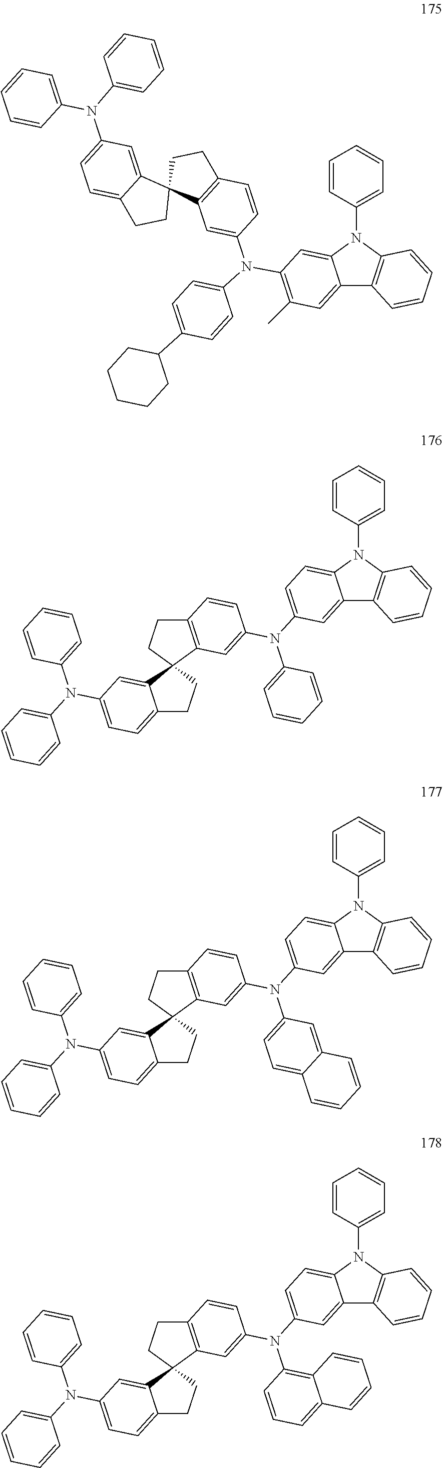 [SMILES: Cc1cc2c3ccccc3n(-c3ccccc3)c2cc1N(c1ccc(C2CCCCC2)cc1)c1ccc2c(c1)[C@]1(CCc3ccc(N(c4ccccc4)c4ccccc4)cc31)CC2 ; c1ccc(N(c2ccccc2)c2ccc3c(c2)[C@@]2(CC3)CCc3ccc(N(c4ccccc4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5ccccc5c4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5c(c4)c4ccccc4n5-c4ccccc4)c4cccc5ccccc45)cc32)cc1]

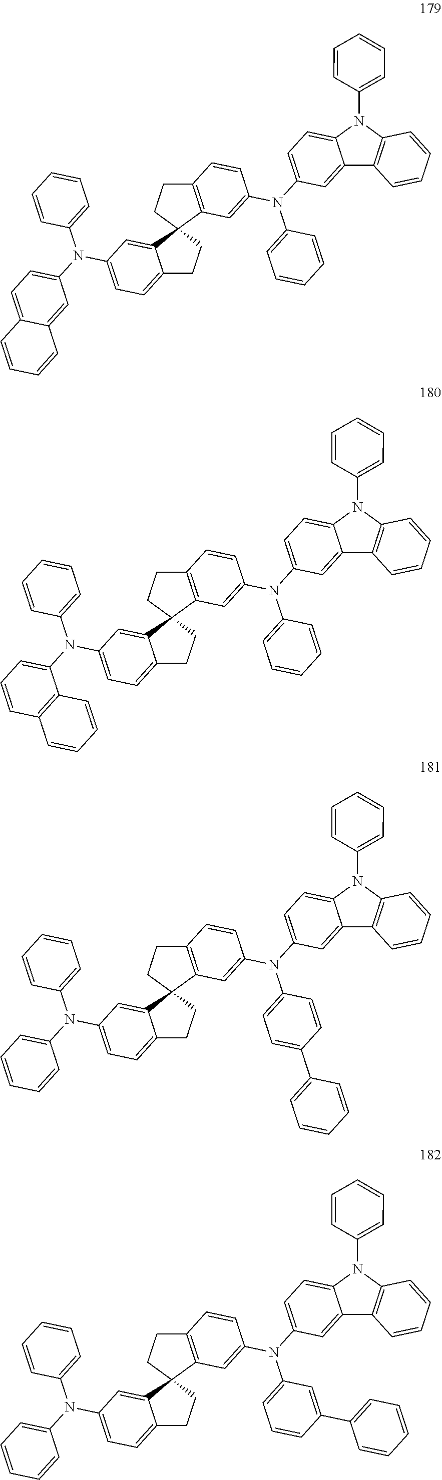 [SMILES: c1ccc(N(c2ccc3c(c2)[C@@]2(CC3)CCc3ccc(N(c4ccccc4)c4ccc5c(c4)c4ccccc4n5-c4ccccc4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)c2ccccc2n3-c2ccccc2)cc1 ; c1ccc(-c2ccc(N(c3ccc4c(c3)[C@]3(CCc5ccc(N(c6ccccc6)c6ccccc6)cc53)CC4)c3ccc4c(c3)c3ccccc3n4-c3ccccc3)cc2)cc1 ; c1ccc(-c2cccc(N(c3ccc4c(c3)[C@]3(CCc5ccc(N(c6ccccc6)c6ccccc6)cc53)CC4)c3ccc4c(c3)c3ccccc3n4-c3ccccc3)c2)cc1]

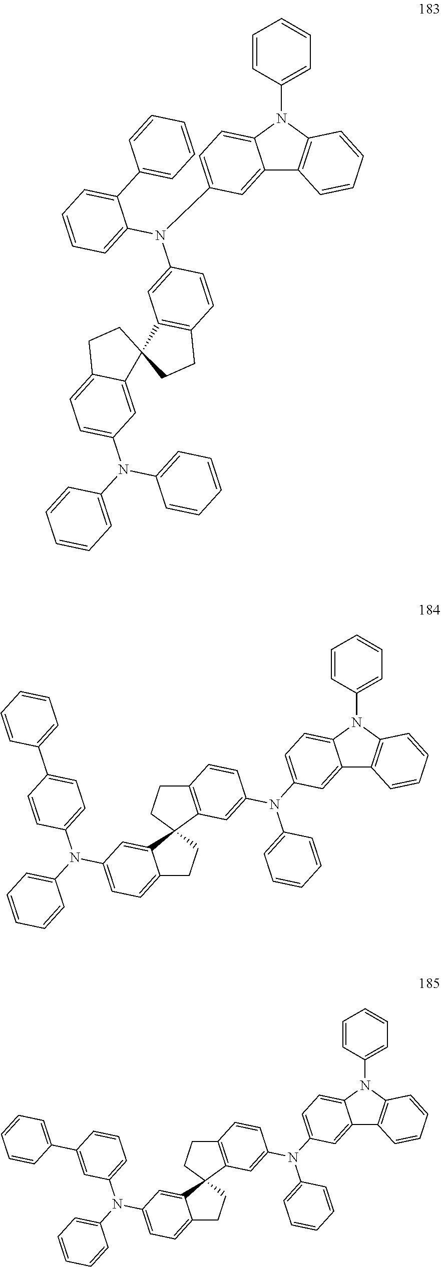 [SMILES: c1ccc(-c2ccccc2N(c2ccc3c(c2)[C@]2(CCc4ccc(N(c5ccccc5)c5ccccc5)cc42)CC3)c2ccc3c(c2)c2ccccc2n3-c2ccccc2)cc1 ; c1ccc(-c2ccc(N(c3ccccc3)c3ccc4c(c3)[C@@]3(CC4)CCc4ccc(N(c5ccccc5)c5ccc6c(c5)c5ccccc5n6-c5ccccc5)cc43)cc2)cc1 ; c1ccc(-c2cccc(N(c3ccccc3)c3ccc4c(c3)[C@@]3(CC4)CCc4ccc(N(c5ccccc5)c5ccc6c(c5)c5ccccc5n6-c5ccccc5)cc43)c2)cc1]

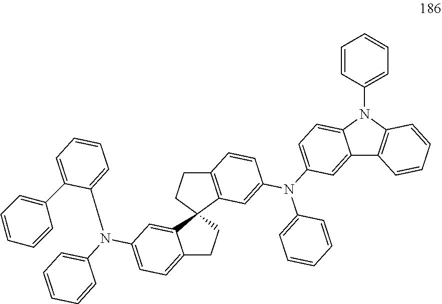 [SMILES: c1ccc(-c2ccccc2N(c2ccccc2)c2ccc3c(c2)[C@]2(CCc4ccc(N(c5ccccc5)c5ccc6c(c5)c5ccccc5n6-c5ccccc5)cc42)CC3)cc1]

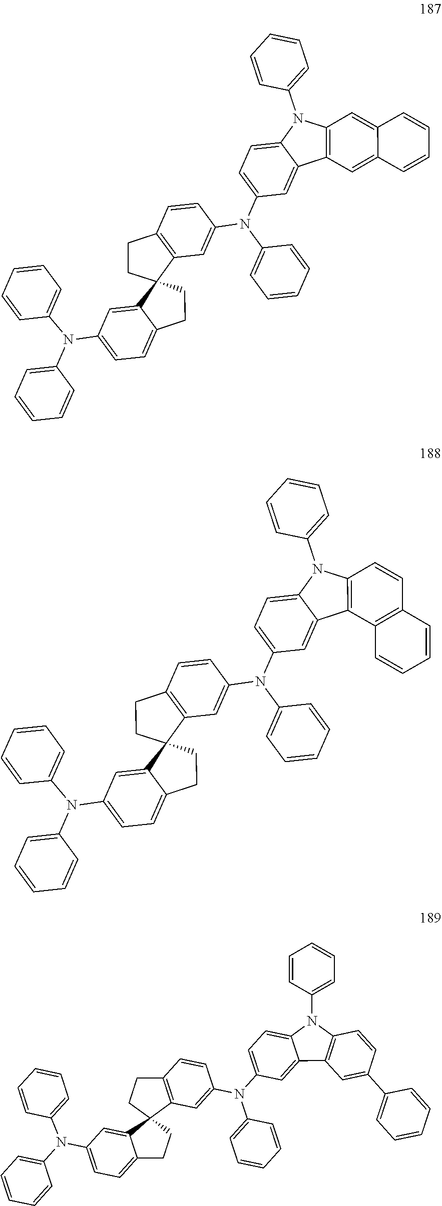 [SMILES: c1ccc(N(c2ccccc2)c2ccc3c(c2)[C@@]2(CC3)CCc3ccc(N(c4ccccc4)c4ccc5c(c4)c4cc6ccccc6cc4n5-c4ccccc4)cc32)cc1 ; c1ccc(N(c2ccccc2)c2ccc3c(c2)[C@@]2(CC3)CCc3ccc(N(c4ccccc4)c4ccc5c(c4)c4c6ccccc6ccc4n5-c4ccccc4)cc32)cc1 ; c1ccc(-c2ccc3c(c2)c2cc(N(c4ccccc4)c4ccc5c(c4)[C@]4(CCc6ccc(N(c7ccccc7)c7ccccc7)cc64)CC5)ccc2n3-c2ccccc2)cc1]

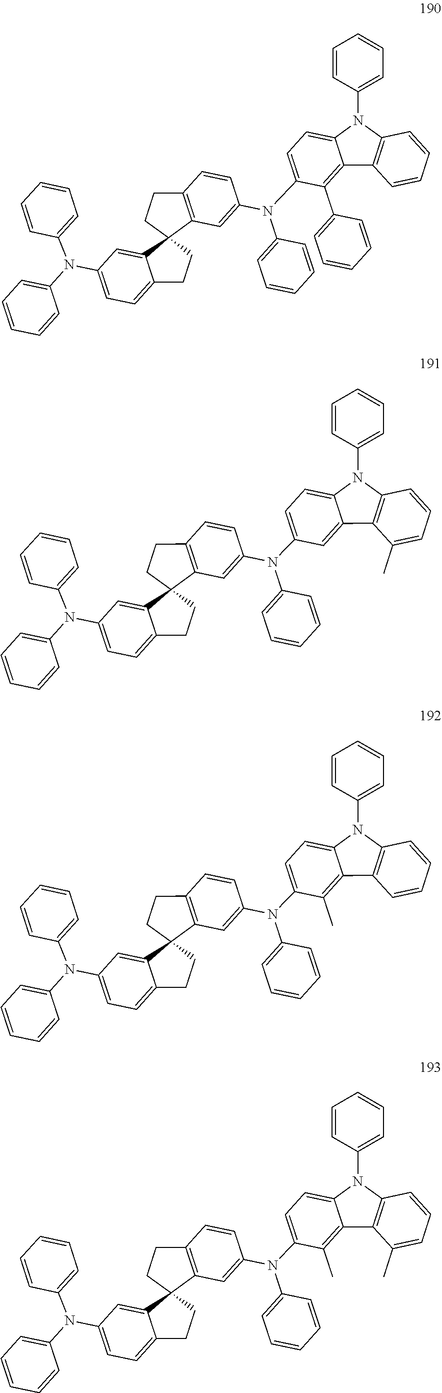 [SMILES: c1ccc(-c2c(N(c3ccccc3)c3ccc4c(c3)[C@]3(CCc5ccc(N(c6ccccc6)c6ccccc6)cc53)CC4)ccc3c2c2ccccc2n3-c2ccccc2)cc1 ; Cc1cccc2c1c1cc(N(c3ccccc3)c3ccc4c(c3)[C@]3(CCc5ccc(N(c6ccccc6)c6ccccc6)cc53)CC4)ccc1n2-c1ccccc1 ; Cc1c(N(c2ccccc2)c2ccc3c(c2)[C@]2(CCc4ccc(N(c5ccccc5)c5ccccc5)cc42)CC3)ccc2c1c1ccccc1n2-c1ccccc1 ; Cc1cccc2c1c1c(C)c(N(c3ccccc3)c3ccc4c(c3)[C@]3(CCc5ccc(N(c6ccccc6)c6ccccc6)cc53)CC4)ccc1n2-c1ccccc1]

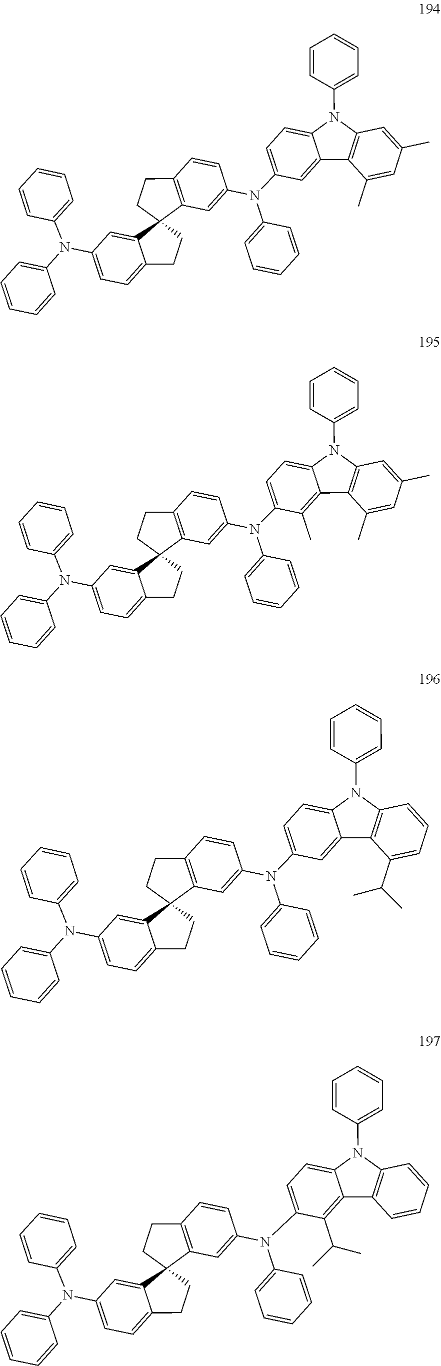 [SMILES: Cc1cc(C)c2c3cc(N(c4ccccc4)c4ccc5c(c4)[C@]4(CCc6ccc(N(c7ccccc7)c7ccccc7)cc64)CC5)ccc3n(-c3ccccc3)c2c1 ; Cc1cc(C)c2c3c(C)c(N(c4ccccc4)c4ccc5c(c4)[C@]4(CCc6ccc(N(c7ccccc7)c7ccccc7)cc64)CC5)ccc3n(-c3ccccc3)c2c1 ; CC(C)c1cccc2c1c1cc(N(c3ccccc3)c3ccc4c(c3)[C@]3(CCc5ccc(N(c6ccccc6)c6ccccc6)cc53)CC4)ccc1n2-c1ccccc1 ; CC(C)c1c(N(c2ccccc2)c2ccc3c(c2)[C@]2(CCc4ccc(N(c5ccccc5)c5ccccc5)cc42)CC3)ccc2c1c1ccccc1n2-c1ccccc1]

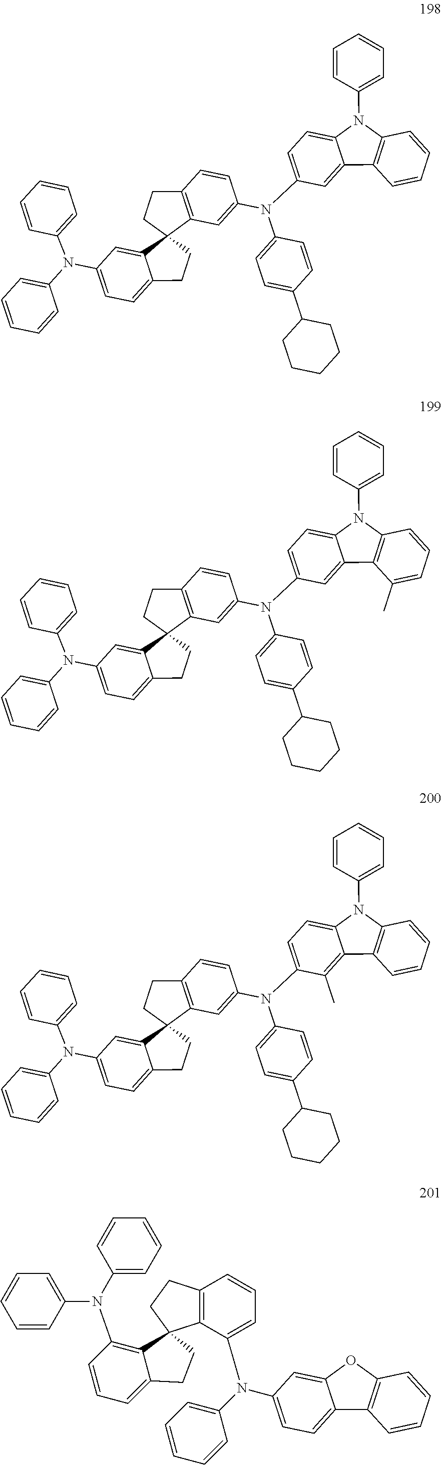 [SMILES: c1ccc(N(c2ccccc2)c2ccc3c(c2)[C@@]2(CC3)CCc3ccc(N(c4ccc(C5CCCCC5)cc4)c4ccc5c(c4)c4ccccc4n5-c4ccccc4)cc32)cc1 ; Cc1cccc2c1c1cc(N(c3ccc(C4CCCCC4)cc3)c3ccc4c(c3)[C@]3(CCc5ccc(N(c6ccccc6)c6ccccc6)cc53)CC4)ccc1n2-c1ccccc1 ; Cc1c(N(c2ccc(C3CCCCC3)cc2)c2ccc3c(c2)[C@]2(CCc4ccc(N(c5ccccc5)c5ccccc5)cc42)CC3)ccc2c1c1ccccc1n2-c1ccccc1 ; c1ccc(N(c2ccccc2)c2cccc3c2[C@@]2(CC3)CCc3cccc(N(c4ccccc4)c4ccc5c(c4)oc4ccccc45)c32)cc1]

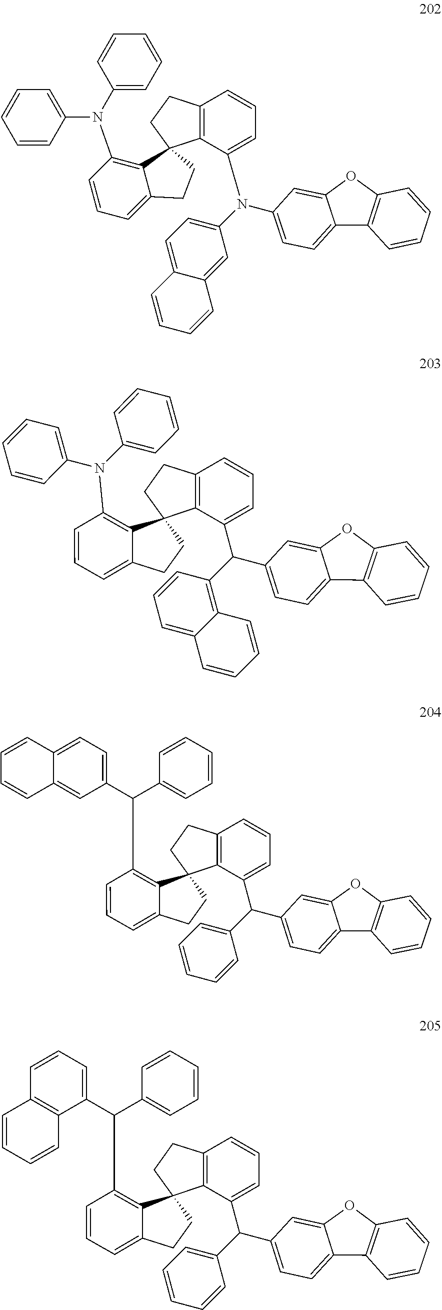 [SMILES: c1ccc(N(c2ccccc2)c2cccc3c2[C@@]2(CC3)CCc3cccc(N(c4ccc5ccccc5c4)c4ccc5c(c4)oc4ccccc45)c32)cc1 ; c1ccc(N(c2ccccc2)c2cccc3c2[C@]2(CCc4cccc(C(c5ccc6c(c5)oc5ccccc56)c5cccc6ccccc56)c42)CC3)cc1 ; c1ccc(C(c2ccc3ccccc3c2)c2cccc3c2[C@@]2(CC3)CCc3cccc(C(c4ccccc4)c4ccc5c(c4)oc4ccccc45)c32)cc1 ; c1ccc(C(c2ccc3c(c2)oc2ccccc23)c2cccc3c2[C@@]2(CC3)CCc3cccc(C(c4ccccc4)c4cccc5ccccc45)c32)cc1]

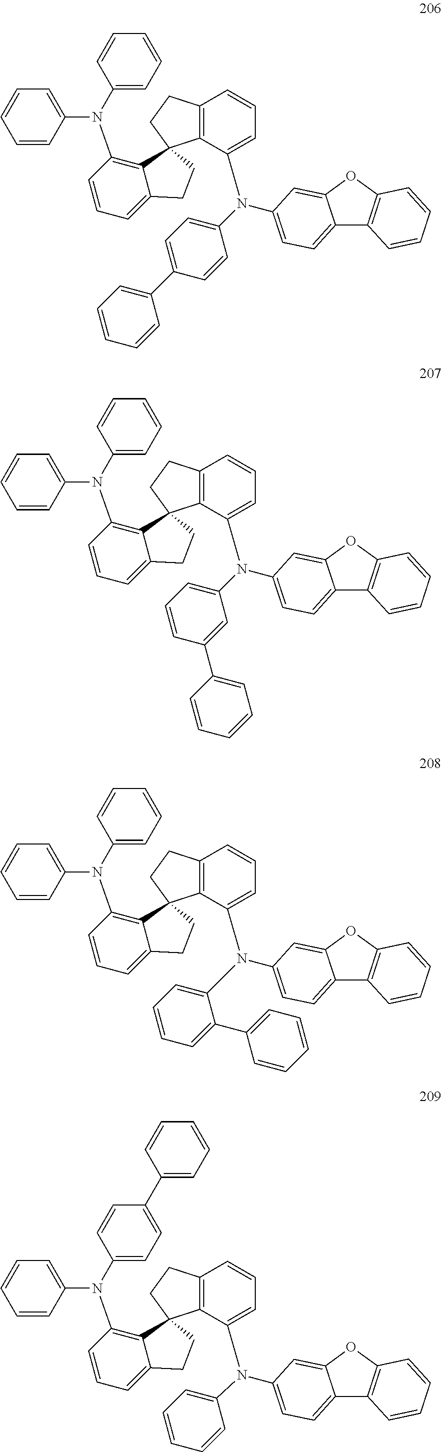 [SMILES: c1ccc(-c2ccc(N(c3ccc4c(c3)oc3ccccc34)c3cccc4c3[C@]3(CCc5cccc(N(c6ccccc6)c6ccccc6)c53)CC4)cc2)cc1 ; c1ccc(-c2cccc(N(c3ccc4c(c3)oc3ccccc34)c3cccc4c3[C@]3(CCc5cccc(N(c6ccccc6)c6ccccc6)c53)CC4)c2)cc1 ; c1ccc(-c2ccccc2N(c2ccc3c(c2)oc2ccccc23)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6c(c5)oc5ccccc56)c43)cc2)cc1]

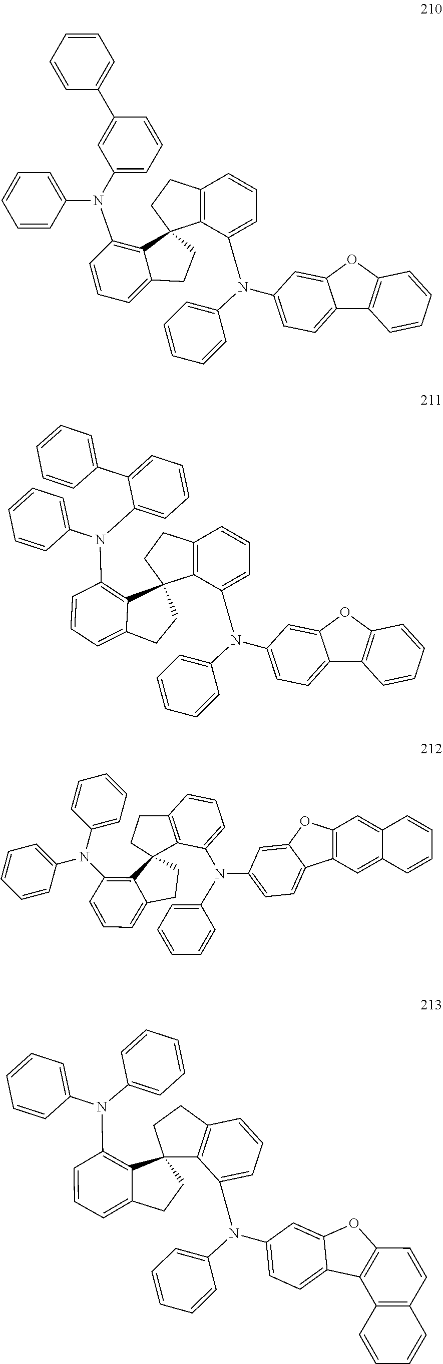 [SMILES: c1ccc(-c2cccc(N(c3ccccc3)c3cccc4c3[C@@]3(CC4)CCc4cccc(N(c5ccccc5)c5ccc6c(c5)oc5ccccc56)c43)c2)cc1 ; c1ccc(-c2ccccc2N(c2ccccc2)c2cccc3c2[C@]2(CCc4cccc(N(c5ccccc5)c5ccc6c(c5)oc5ccccc56)c42)CC3)cc1 ; c1ccc(N(c2ccccc2)c2cccc3c2[C@@]2(CC3)CCc3cccc(N(c4ccccc4)c4ccc5c(c4)oc4cc6ccccc6cc45)c32)cc1 ; c1ccc(N(c2ccccc2)c2cccc3c2[C@@]2(CC3)CCc3cccc(N(c4ccccc4)c4ccc5c(c4)oc4ccc6ccccc6c45)c32)cc1]

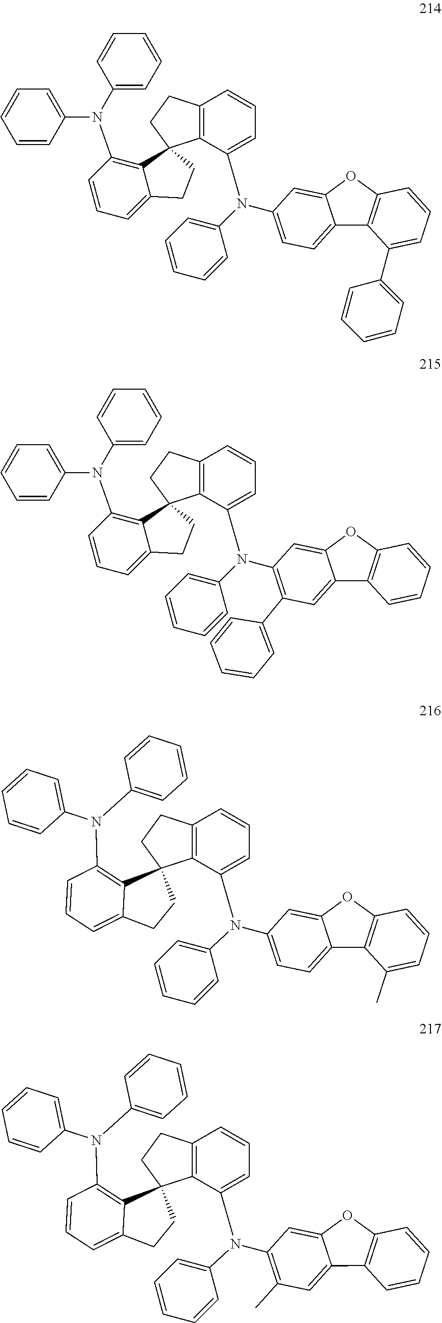 [SMILES: c1ccc(-c2cccc3oc4cc(N(c5ccccc5)c5cccc6c5[C@]5(CCc7cccc(N(c8ccccc8)c8ccccc8)c75)CC6)ccc4c23)cc1 ; c1ccc(-c2cc3c(cc2N(c2ccccc2)c2cccc4c2[C@]2(CCc5cccc(N(c6ccccc6)c6ccccc6)c52)CC4)oc2ccccc23)cc1 ; Cc1cccc2oc3cc(N(c4ccccc4)c4cccc5c4[C@]4(CCc6cccc(N(c7ccccc7)c7ccccc7)c64)CC5)ccc3c12 ; Cc1cc2c(cc1N(c1ccccc1)c1cccc3c1[C@]1(CCc4cccc(N(c5ccccc5)c5ccccc5)c41)CC3)oc1ccccc12]

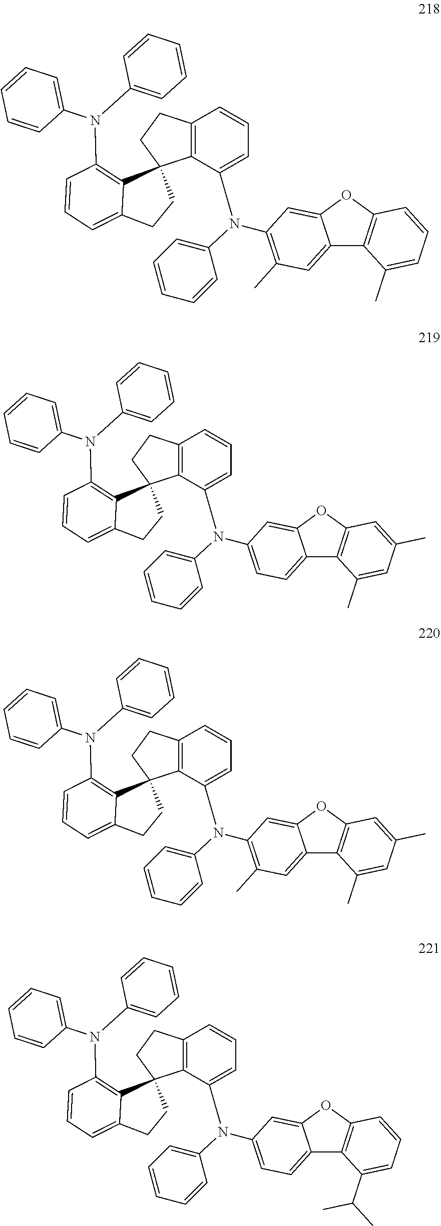 [SMILES: Cc1cc2c(cc1N(c1ccccc1)c1cccc3c1[C@]1(CCc4cccc(N(c5ccccc5)c5ccccc5)c41)CC3)oc1cccc(C)c12 ; Cc1cc(C)c2c(c1)oc1cc(N(c3ccccc3)c3cccc4c3[C@]3(CCc5cccc(N(c6ccccc6)c6ccccc6)c53)CC4)ccc12 ; Cc1cc(C)c2c(c1)oc1cc(N(c3ccccc3)c3cccc4c3[C@]3(CCc5cccc(N(c6ccccc6)c6ccccc6)c53)CC4)c(C)cc12 ; CC(C)c1cccc2oc3cc(N(c4ccccc4)c4cccc5c4[C@]4(CCc6cccc(N(c7ccccc7)c7ccccc7)c64)CC5)ccc3c12]

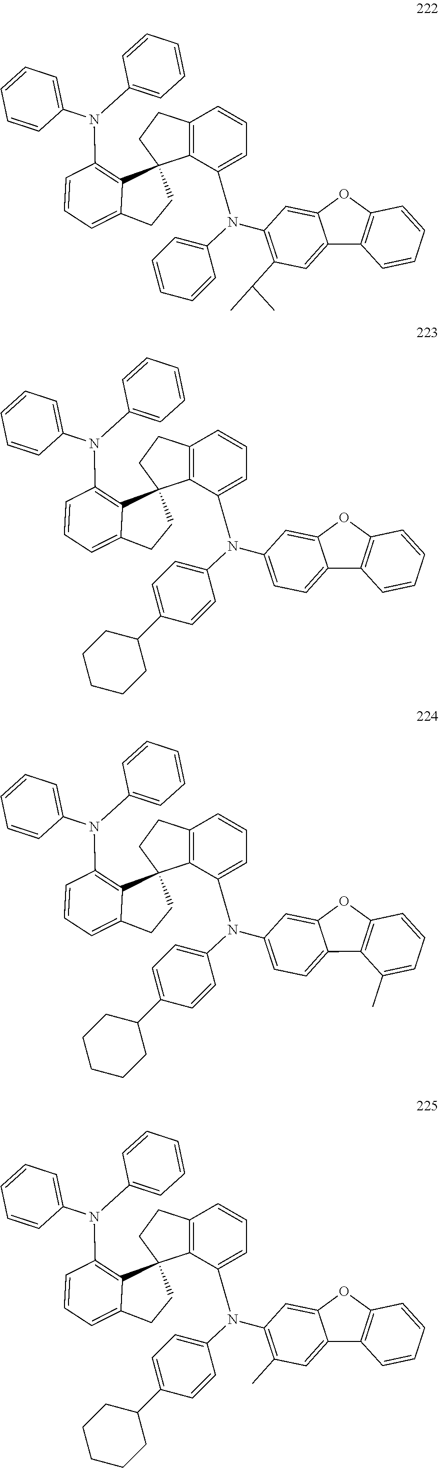 [SMILES: CC(C)c1cc2c(cc1N(c1ccccc1)c1cccc3c1[C@]1(CCc4cccc(N(c5ccccc5)c5ccccc5)c41)CC3)oc1ccccc12 ; c1ccc(N(c2ccccc2)c2cccc3c2[C@@]2(CC3)CCc3cccc(N(c4ccc(C5CCCCC5)cc4)c4ccc5c(c4)oc4ccccc45)c32)cc1 ; Cc1cccc2oc3cc(N(c4ccc(C5CCCCC5)cc4)c4cccc5c4[C@]4(CCc6cccc(N(c7ccccc7)c7ccccc7)c64)CC5)ccc3c12 ; Cc1cc2c(cc1N(c1ccc(C3CCCCC3)cc1)c1cccc3c1[C@]1(CCc4cccc(N(c5ccccc5)c5ccccc5)c41)CC3)oc1ccccc12]

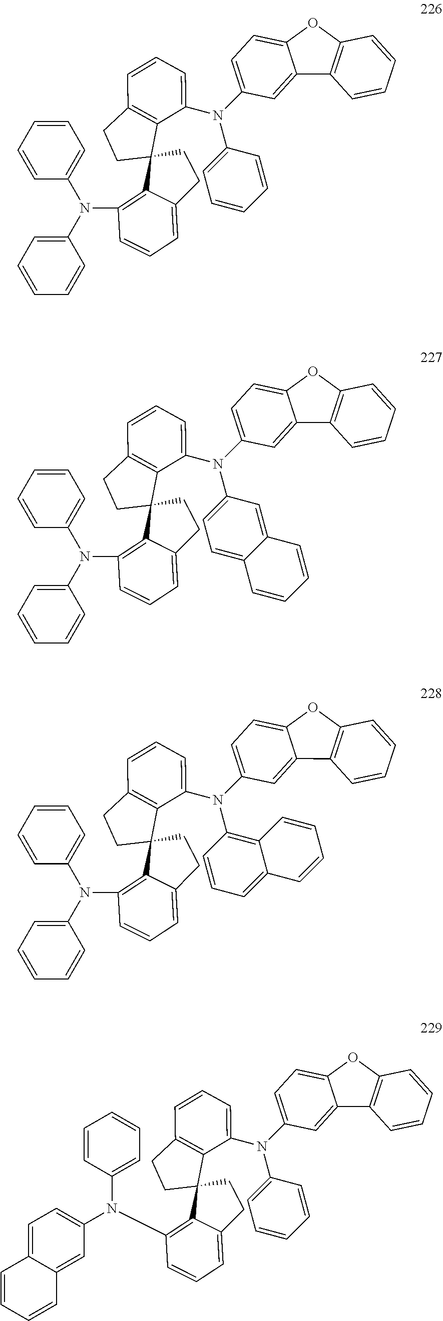 [SMILES: c1ccc(N(c2ccccc2)c2cccc3c2[C@@]2(CC3)CCc3cccc(N(c4ccccc4)c4ccc5oc6ccccc6c5c4)c32)cc1 ; c1ccc(N(c2ccccc2)c2cccc3c2[C@@]2(CC3)CCc3cccc(N(c4ccc5ccccc5c4)c4ccc5oc6ccccc6c5c4)c32)cc1 ; c1ccc(N(c2ccccc2)c2cccc3c2[C@@]2(CC3)CCc3cccc(N(c4ccc5oc6ccccc6c5c4)c4cccc5ccccc45)c32)cc1 ; c1ccc(N(c2ccc3ccccc3c2)c2cccc3c2[C@@]2(CC3)CCc3cccc(N(c4ccccc4)c4ccc5oc6ccccc6c5c4)c32)cc1]

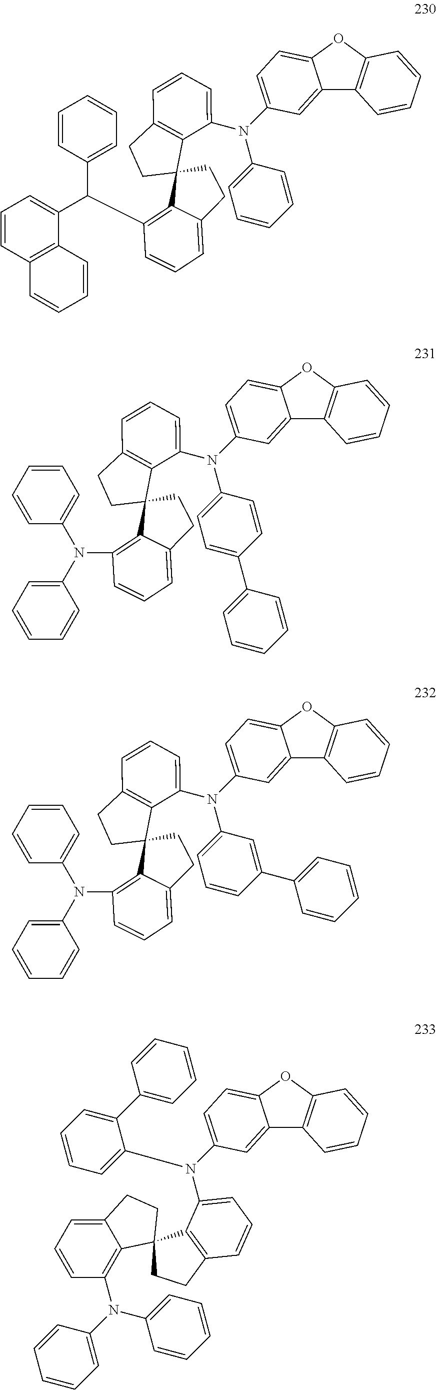 [SMILES: c1ccc(C(c2cccc3c2[C@@]2(CC3)CCc3cccc(N(c4ccccc4)c4ccc5oc6ccccc6c5c4)c32)c2cccc3ccccc23)cc1 ; c1ccc(-c2ccc(N(c3ccc4oc5ccccc5c4c3)c3cccc4c3[C@]3(CCc5cccc(N(c6ccccc6)c6ccccc6)c53)CC4)cc2)cc1 ; c1ccc(-c2cccc(N(c3ccc4oc5ccccc5c4c3)c3cccc4c3[C@]3(CCc5cccc(N(c6ccccc6)c6ccccc6)c53)CC4)c2)cc1 ; c1ccc(-c2ccccc2N(c2ccc3oc4ccccc4c3c2)c2cccc3c2[C@]2(CCc4cccc(N(c5ccccc5)c5ccccc5)c42)CC3)cc1]

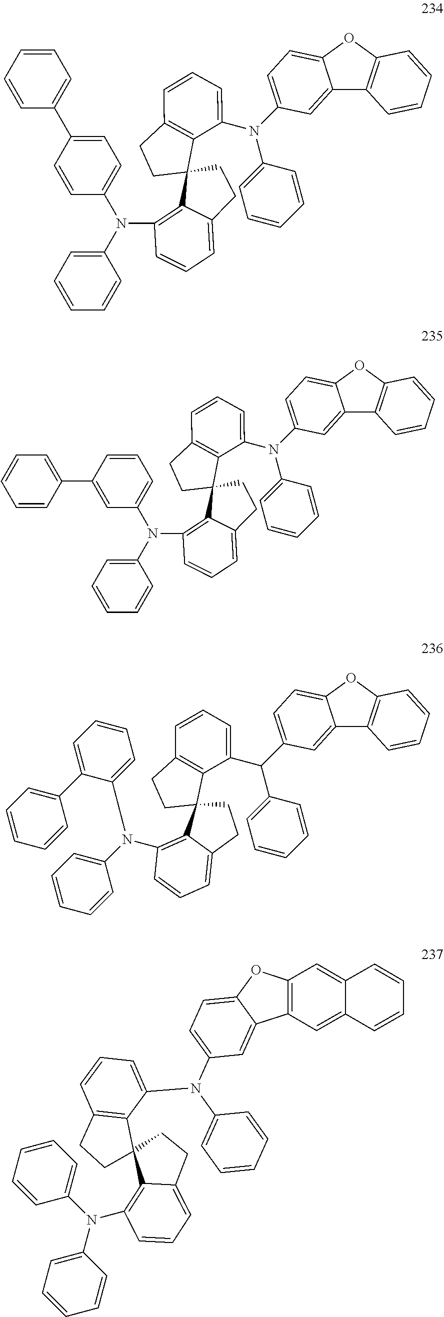

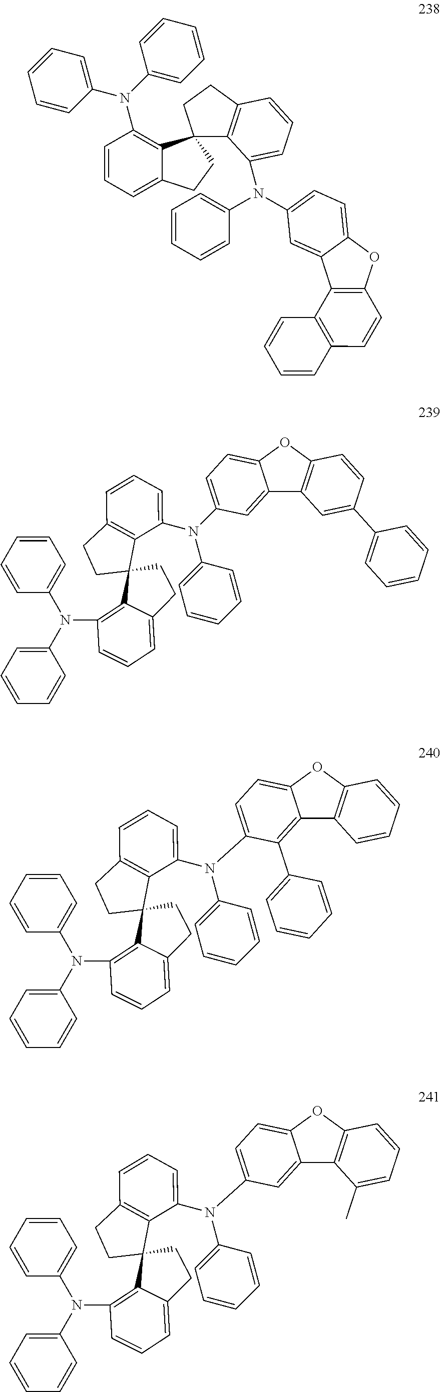 [SMILES: c1ccc(N(c2ccccc2)c2cccc3c2[C@@]2(CC3)CCc3cccc(N(c4ccccc4)c4ccc5oc6ccc7ccccc7c6c5c4)c32)cc1 ; c1ccc(-c2ccc3oc4ccc(N(c5ccccc5)c5cccc6c5[C@]5(CCc7cccc(N(c8ccccc8)c8ccccc8)c75)CC6)cc4c3c2)cc1 ; c1ccc(-c2c(N(c3ccccc3)c3cccc4c3[C@]3(CCc5cccc(N(c6ccccc6)c6ccccc6)c53)CC4)ccc3oc4ccccc4c23)cc1 ; Cc1cccc2oc3ccc(N(c4ccccc4)c4cccc5c4[C@]4(CCc6cccc(N(c7ccccc7)c7ccccc7)c64)CC5)cc3c12]

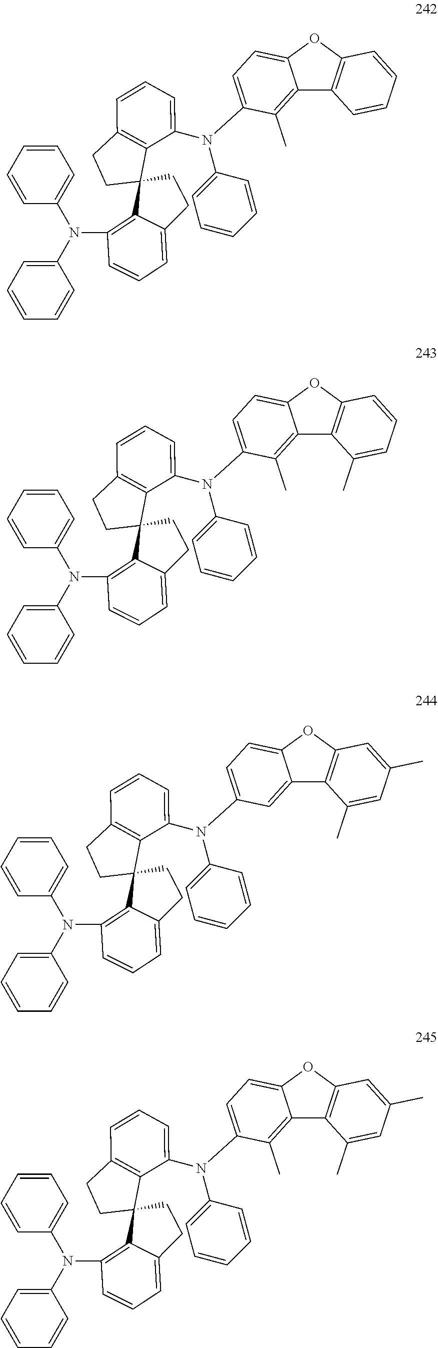 [SMILES: Cc1c(N(c2ccccc2)c2cccc3c2[C@]2(CCc4cccc(N(c5ccccc5)c5ccccc5)c42)CC3)ccc2oc3ccccc3c12 ; Cc1cccc2oc3ccc(N(c4ccccc4)c4cccc5c4[C@]4(CCc6cccc(N(c7ccccc7)c7ccccc7)c64)CC5)c(C)c3c12 ; Cc1cc(C)c2c(c1)oc1ccc(N(c3ccccc3)c3cccc4c3[C@]3(CCc5cccc(N(c6ccccc6)c6ccccc6)c53)CC4)cc12 ; Cc1cc(C)c2c(c1)oc1ccc(N(c3ccccc3)c3cccc4c3[C@]3(CCc5cccc(N(c6ccccc6)c6ccccc6)c53)CC4)c(C)c12]

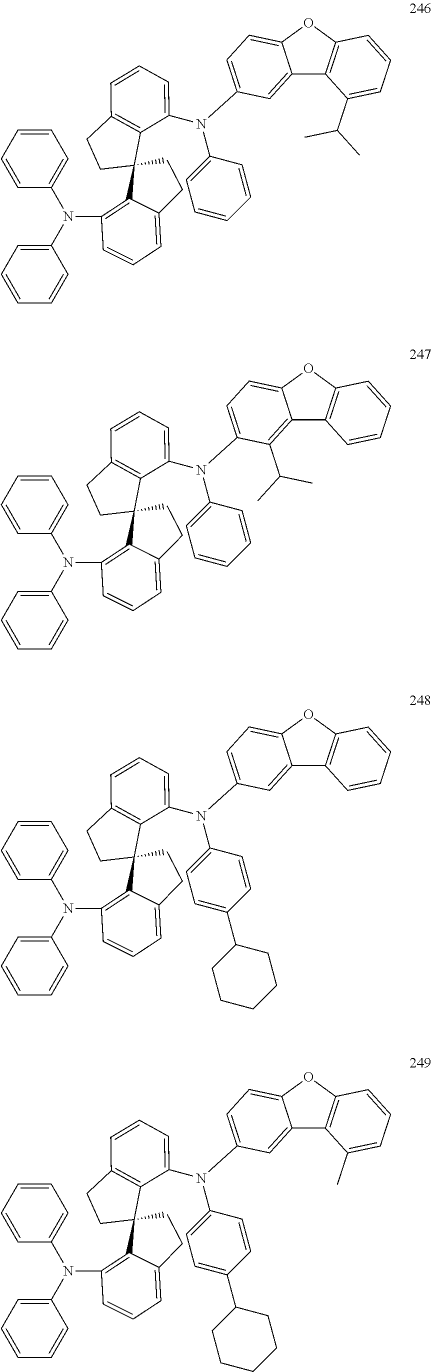 [SMILES: CC(C)c1cccc2oc3ccc(N(c4ccccc4)c4cccc5c4[C@]4(CCc6cccc(N(c7ccccc7)c7ccccc7)c64)CC5)cc3c12 ; CC(C)c1c(N(c2ccccc2)c2cccc3c2[C@]2(CCc4cccc(N(c5ccccc5)c5ccccc5)c42)CC3)ccc2oc3ccccc3c12 ; c1ccc(N(c2ccccc2)c2cccc3c2[C@@]2(CC3)CCc3cccc(N(c4ccc(C5CCCCC5)cc4)c4ccc5oc6ccccc6c5c4)c32)cc1 ; Cc1cccc2oc3ccc(N(c4ccc(C5CCCCC5)cc4)c4cccc5c4[C@]4(CCc6cccc(N(c7ccccc7)c7ccccc7)c64)CC5)cc3c12]

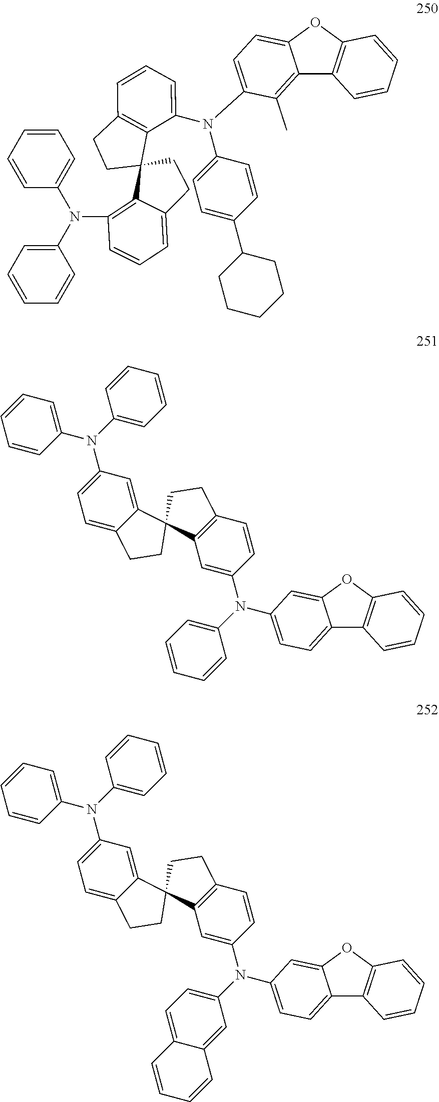 [SMILES: Cc1c(N(c2ccc(C3CCCCC3)cc2)c2cccc3c2[C@]2(CCc4cccc(N(c5ccccc5)c5ccccc5)c42)CC3)ccc2oc3ccccc3c12 ; c1ccc(N(c2ccccc2)c2ccc3c(c2)[C@@]2(CC3)CCc3ccc(N(c4ccccc4)c4ccc5c(c4)oc4ccccc45)cc32)cc1 ; c1ccc(N(c2ccccc2)c2ccc3c(c2)[C@@]2(CC3)CCc3ccc(N(c4ccc5ccccc5c4)c4ccc5c(c4)oc4ccccc45)cc32)cc1]

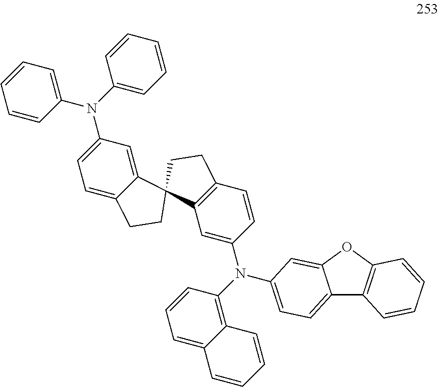 [SMILES: c1ccc(N(c2ccccc2)c2ccc3c(c2)[C@@]2(CC3)CCc3ccc(N(c4ccc5c(c4)oc4ccccc45)c4cccc5ccccc45)cc32)cc1]

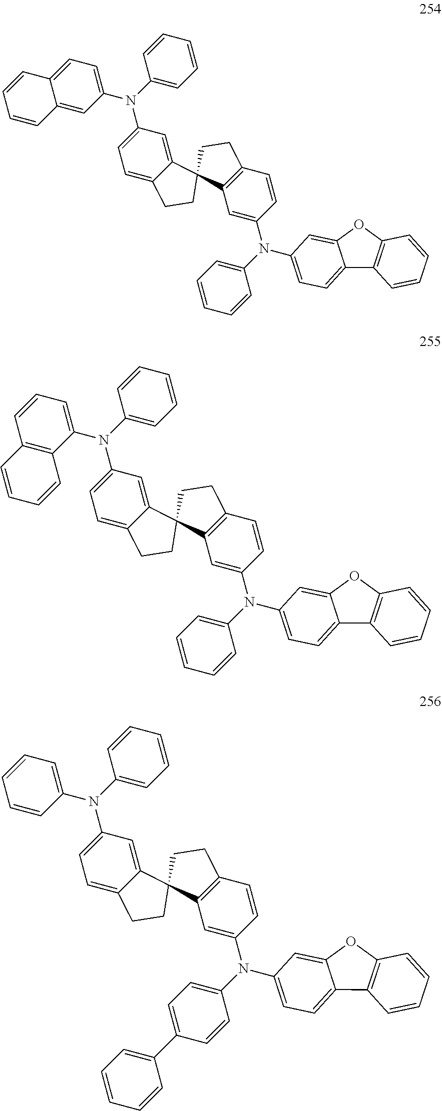 [SMILES: c1ccc(N(c2ccc3c(c2)[C@@]2(CC3)CCc3ccc(N(c4ccccc4)c4ccc5c(c4)oc4ccccc45)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)oc2ccccc23)cc1 ; c1ccc(-c2ccc(N(c3ccc4c(c3)[C@]3(CCc5ccc(N(c6ccccc6)c6ccccc6)cc53)CC4)c3ccc4c(c3)oc3ccccc34)cc2)cc1]

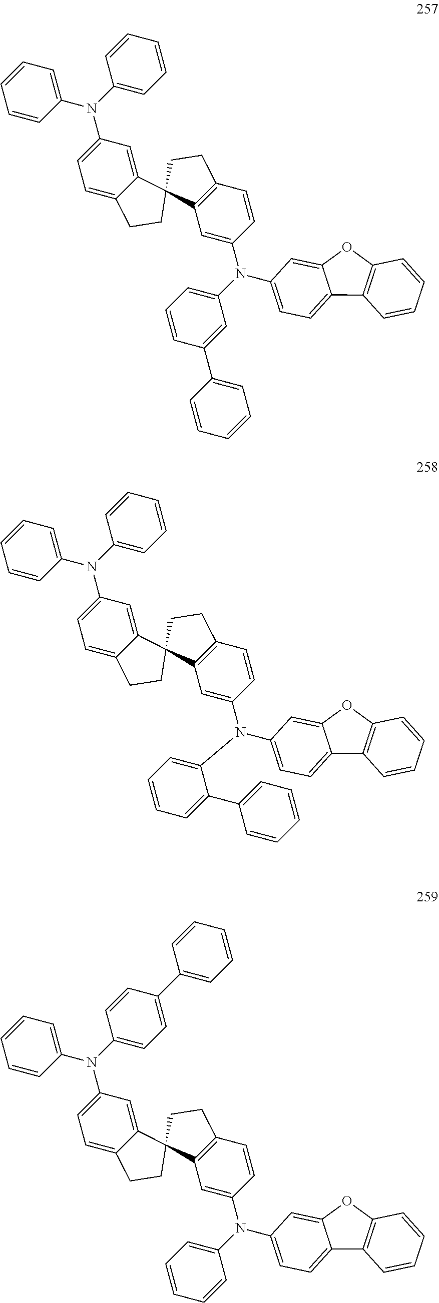 [SMILES: c1ccc(-c2cccc(N(c3ccc4c(c3)[C@]3(CCc5ccc(N(c6ccccc6)c6ccccc6)cc53)CC4)c3ccc4c(c3)oc3ccccc34)c2)cc1 ; c1ccc(-c2ccccc2N(c2ccc3c(c2)[C@]2(CCc4ccc(N(c5ccccc5)c5ccccc5)cc42)CC3)c2ccc3c(c2)oc2ccccc23)cc1 ; c1ccc(-c2ccc(N(c3ccccc3)c3ccc4c(c3)[C@@]3(CC4)CCc4ccc(N(c5ccccc5)c5ccc6c(c5)oc5ccccc56)cc43)cc2)cc1]

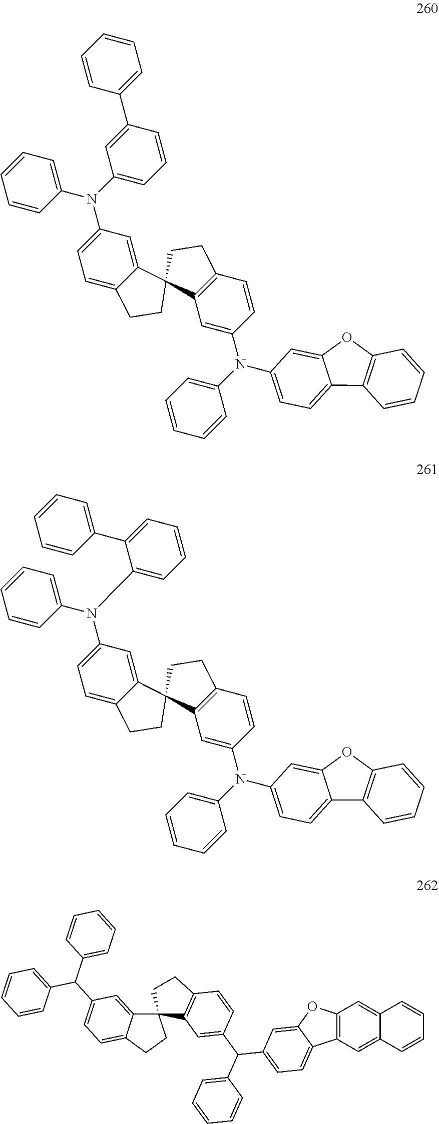 [SMILES: c1ccc(-c2cccc(N(c3ccccc3)c3ccc4c(c3)[C@@]3(CC4)CCc4ccc(N(c5ccccc5)c5ccc6c(c5)oc5ccccc56)cc43)c2)cc1 ; c1ccc(-c2ccccc2N(c2ccccc2)c2ccc3c(c2)[C@]2(CCc4ccc(N(c5ccccc5)c5ccc6c(c5)oc5ccccc56)cc42)CC3)cc1 ; c1ccc(C(c2ccccc2)c2ccc3c(c2)[C@@]2(CC3)CCc3ccc(C(c4ccccc4)c4ccc5c(c4)oc4cc6ccccc6cc45)cc32)cc1]

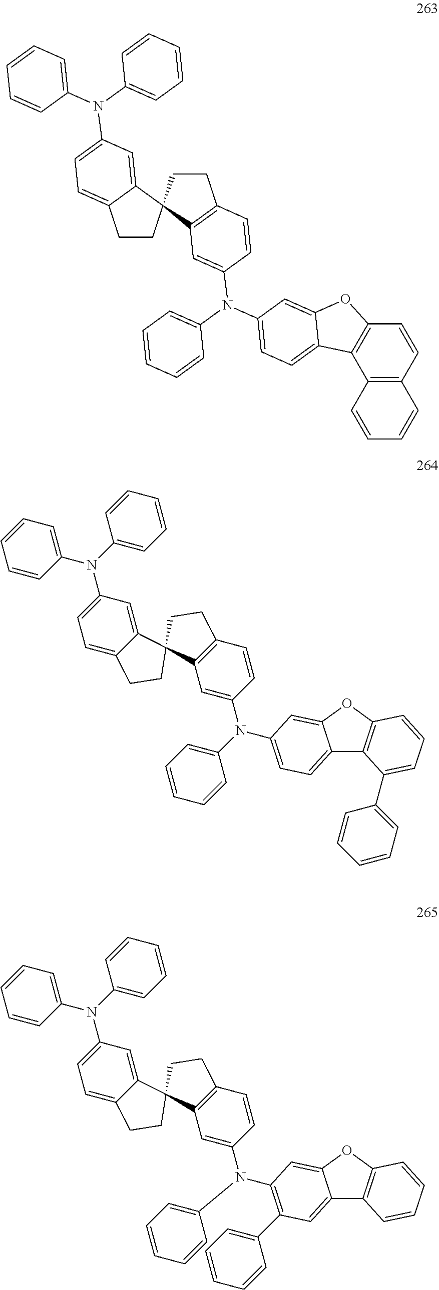 [SMILES: c1ccc(N(c2ccccc2)c2ccc3c(c2)[C@@]2(CC3)CCc3ccc(N(c4ccccc4)c4ccc5c(c4)oc4ccc6ccccc6c45)cc32)cc1 ; c1ccc(-c2cccc3oc4cc(N(c5ccccc5)c5ccc6c(c5)[C@]5(CCc7ccc(N(c8ccccc8)c8ccccc8)cc75)CC6)ccc4c23)cc1 ; c1ccc(-c2cc3c(cc2N(c2ccccc2)c2ccc4c(c2)[C@]2(CCc5ccc(N(c6ccccc6)c6ccccc6)cc52)CC4)oc2ccccc23)cc1]

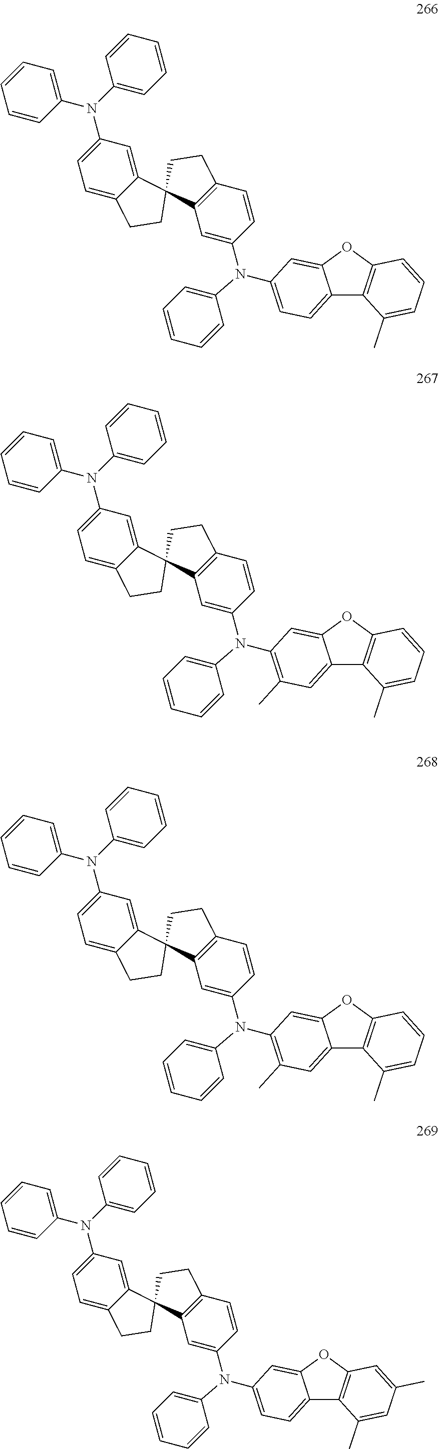 [SMILES: Cc1cccc2oc3cc(N(c4ccccc4)c4ccc5c(c4)[C@]4(CCc6ccc(N(c7ccccc7)c7ccccc7)cc64)CC5)ccc3c12 ; Cc1cc2c(cc1N(c1ccccc1)c1ccc3c(c1)[C@]1(CCc4ccc(N(c5ccccc5)c5ccccc5)cc41)CC3)oc1cccc(C)c12 ; Cc1cc2c(cc1N(c1ccccc1)c1ccc3c(c1)[C@]1(CCc4ccc(N(c5ccccc5)c5ccccc5)cc41)CC3)oc1cccc(C)c12 ; Cc1cc(C)c2c(c1)oc1cc(N(c3ccccc3)c3ccc4c(c3)[C@]3(CCc5ccc(N(c6ccccc6)c6ccccc6)cc53)CC4)ccc12]

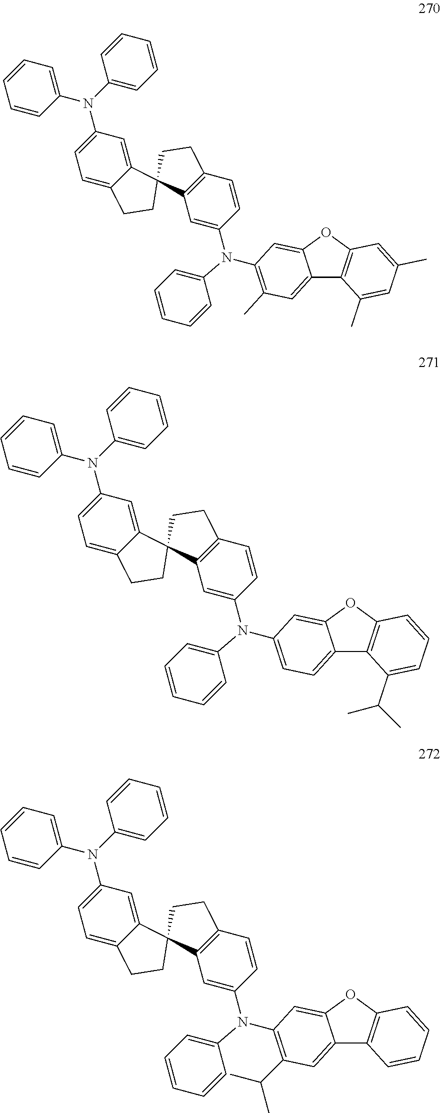 [SMILES: Cc1cc(C)c2c(c1)oc1cc(N(c3ccccc3)c3ccc4c(c3)[C@]3(CCc5ccc(N(c6ccccc6)c6ccccc6)cc53)CC4)c(C)cc12 ; CC(C)c1cccc2oc3cc(N(c4ccccc4)c4ccc5c(c4)[C@]4(CCc6ccc(N(c7ccccc7)c7ccccc7)cc64)CC5)ccc3c12 ; CC(C)c1cc2c(cc1N(c1ccccc1)c1ccc3c(c1)[C@]1(CCc4ccc(N(c5ccccc5)c5ccccc5)cc41)CC3)oc1ccccc12]

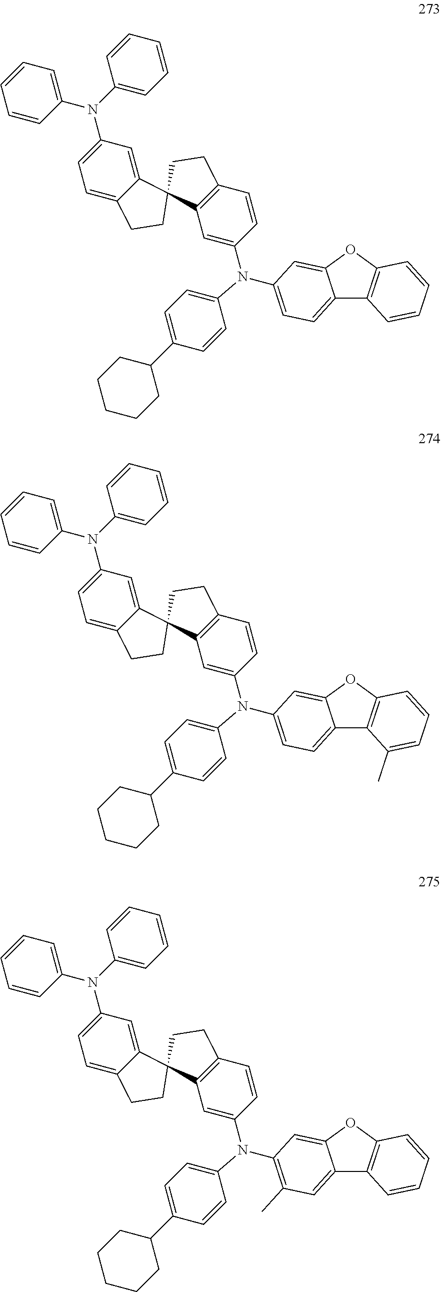 [SMILES: c1ccc(N(c2ccccc2)c2ccc3c(c2)[C@@]2(CC3)CCc3ccc(N(c4ccc(C5CCCCC5)cc4)c4ccc5c(c4)oc4ccccc45)cc32)cc1 ; Cc1cccc2oc3cc(N(c4ccc(C5CCCCC5)cc4)c4ccc5c(c4)[C@]4(CCc6ccc(N(c7ccccc7)c7ccccc7)cc64)CC5)ccc3c12 ; Cc1cc2c(cc1N(c1ccc(C3CCCCC3)cc1)c1ccc3c(c1)[C@]1(CCc4ccc(N(c5ccccc5)c5ccccc5)cc41)CC3)oc1ccccc12]

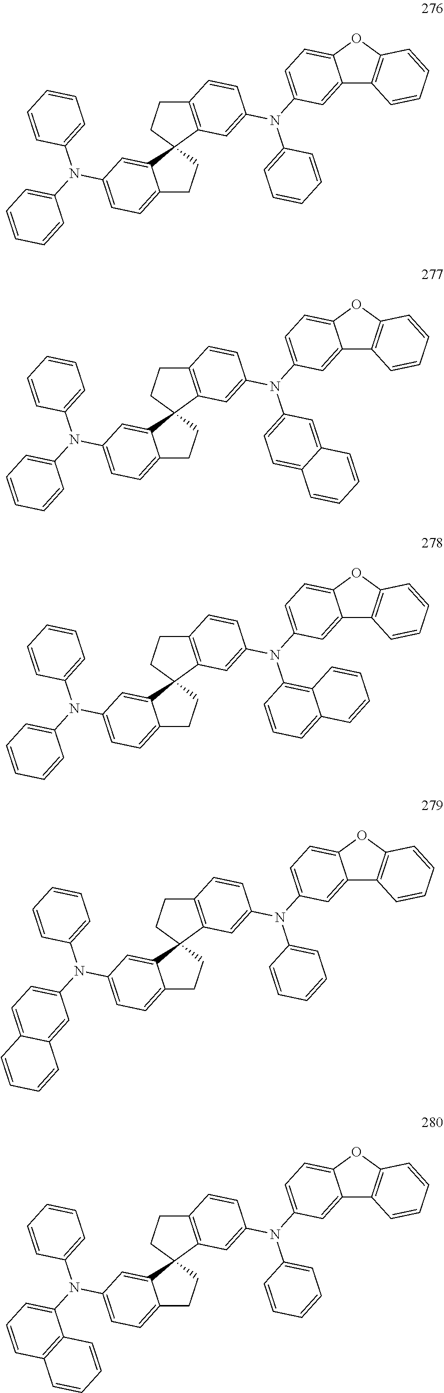 [SMILES: c1ccc(N(c2ccccc2)c2ccc3c(c2)[C@@]2(CC3)CCc3ccc(N(c4ccccc4)c4ccc5oc6ccccc6c5c4)cc32)cc1 ; c1ccc(N(c2ccccc2)c2ccc3c(c2)[C@@]2(CC3)CCc3ccc(N(c4ccc5ccccc5c4)c4ccc5oc6ccccc6c5c4)cc32)cc1 ; c1ccc(N(c2ccccc2)c2ccc3c(c2)[C@@]2(CC3)CCc3ccc(N(c4ccc5oc6ccccc6c5c4)c4cccc5ccccc45)cc32)cc1 ; c1ccc(N(c2ccc3c(c2)[C@@]2(CC3)CCc3ccc(N(c4ccccc4)c4ccc5oc6ccccc6c5c4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3oc4ccccc4c3c2)cc1]

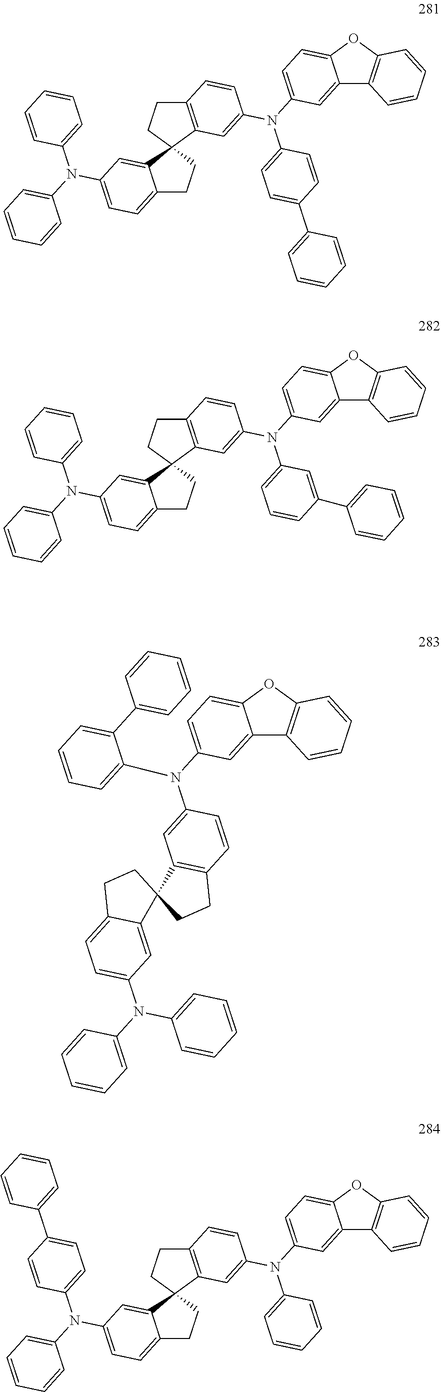 [SMILES: c1ccc(-c2ccc(N(c3ccc4c(c3)[C@]3(CCc5ccc(N(c6ccccc6)c6ccccc6)cc53)CC4)c3ccc4oc5ccccc5c4c3)cc2)cc1 ; c1ccc(-c2cccc(N(c3ccc4c(c3)[C@]3(CCc5ccc(N(c6ccccc6)c6ccccc6)cc53)CC4)c3ccc4oc5ccccc5c4c3)c2)cc1 ; c1ccc(-c2ccccc2N(c2ccc3c(c2)[C@]2(CCc4ccc(N(c5ccccc5)c5ccccc5)cc42)CC3)c2ccc3oc4ccccc4c3c2)cc1 ; c1ccc(-c2ccc(N(c3ccccc3)c3ccc4c(c3)[C@@]3(CC4)CCc4ccc(N(c5ccccc5)c5ccc6oc7ccccc7c6c5)cc43)cc2)cc1]

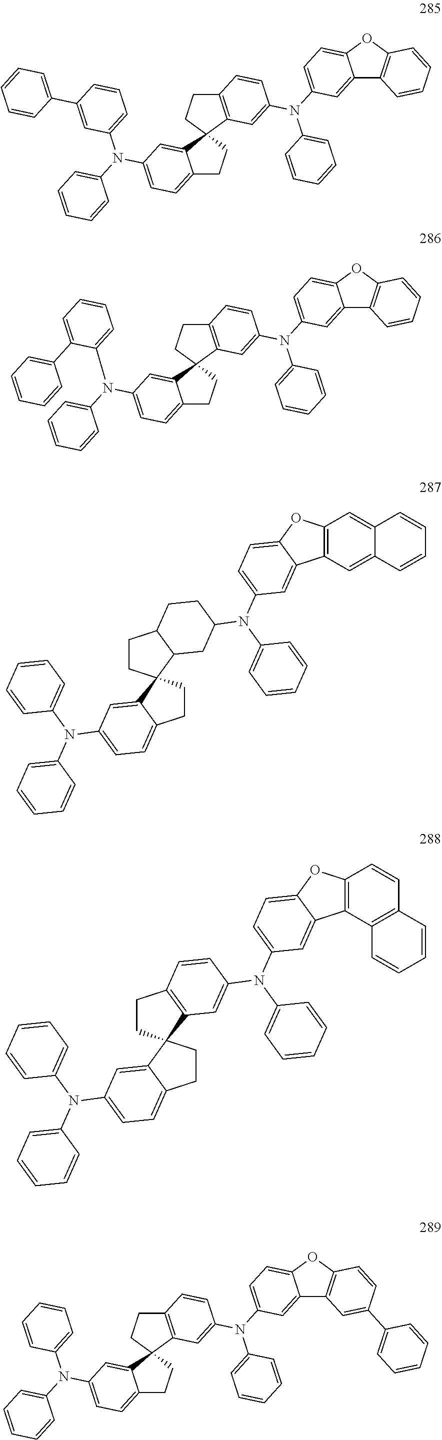 [SMILES: c1ccc(-c2cccc(N(c3ccccc3)c3ccc4c(c3)[C@@]3(CC4)CCc4ccc(N(c5ccccc5)c5ccc6oc7ccccc7c6c5)cc43)c2)cc1 ; c1ccc(-c2ccccc2N(c2ccccc2)c2ccc3c(c2)[C@]2(CCc4ccc(N(c5ccccc5)c5ccc6oc7ccccc7c6c5)cc42)CC3)cc1 ; c1ccc(N(c2ccccc2)c2ccc3c(c2)[C@@]2(CC3)CCC3CCC(N(c4ccccc4)c4ccc5oc6cc7ccccc7cc6c5c4)CC32)cc1 ; c1ccc(N(c2ccccc2)c2ccc3c(c2)[C@@]2(CC3)CCc3ccc(N(c4ccccc4)c4ccc5oc6ccc7ccccc7c6c5c4)cc32)cc1 ; c1ccc(-c2ccc3oc4ccc(N(c5ccccc5)c5ccc6c(c5)[C@]5(CCc7ccc(N(c8ccccc8)c8ccccc8)cc75)CC6)cc4c3c2)cc1]

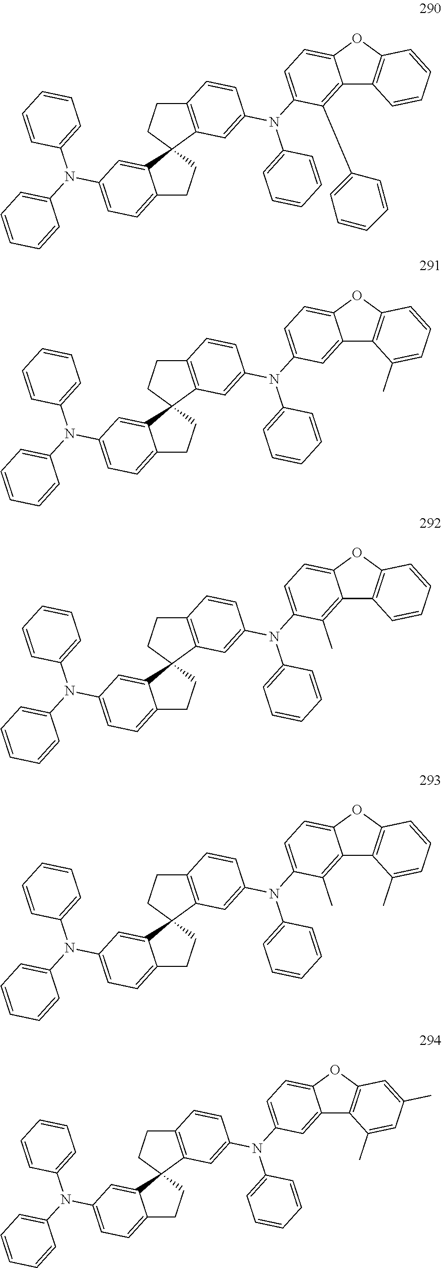 [SMILES: c1ccc(-c2c(N(c3ccccc3)c3ccc4c(c3)[C@]3(CCc5ccc(N(c6ccccc6)c6ccccc6)cc53)CC4)ccc3oc4ccccc4c23)cc1 ; Cc1cccc2oc3ccc(N(c4ccccc4)c4ccc5c(c4)[C@]4(CCc6ccc(N(c7ccccc7)c7ccccc7)cc64)CC5)cc3c12 ; Cc1c(N(c2ccccc2)c2ccc3c(c2)[C@]2(CCc4ccc(N(c5ccccc5)c5ccccc5)cc42)CC3)ccc2oc3ccccc3c12 ; Cc1cccc2oc3ccc(N(c4ccccc4)c4ccc5c(c4)[C@]4(CCc6ccc(N(c7ccccc7)c7ccccc7)cc64)CC5)c(C)c3c12 ; Cc1cc(C)c2c(c1)oc1ccc(N(c3ccccc3)c3ccc4c(c3)[C@]3(CCc5ccc(N(c6ccccc6)c6ccccc6)cc53)CC4)cc12]

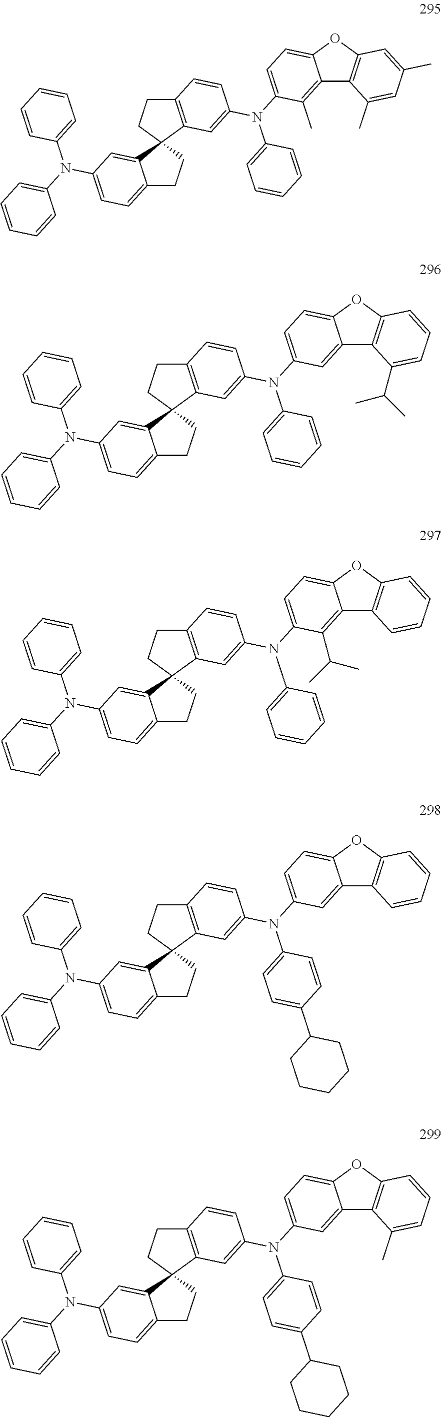 [SMILES: Cc1cc(C)c2c(c1)oc1ccc(N(c3ccccc3)c3ccc4c(c3)[C@]3(CCc5ccc(N(c6ccccc6)c6ccccc6)cc53)CC4)c(C)c12 ; CC(C)c1cccc2oc3ccc(N(c4ccccc4)c4ccc5c(c4)[C@]4(CCc6ccc(N(c7ccccc7)c7ccccc7)cc64)CC5)cc3c12 ; CC(C)c1c(N(c2ccccc2)c2ccc3c(c2)[C@]2(CCc4ccc(N(c5ccccc5)c5ccccc5)cc42)CC3)ccc2oc3ccccc3c12 ; c1ccc(N(c2ccccc2)c2ccc3c(c2)[C@@]2(CC3)CCc3ccc(N(c4ccc(C5CCCCC5)cc4)c4ccc5oc6ccccc6c5c4)cc32)cc1 ; Cc1cccc2oc3ccc(N(c4ccc(C5CCCCC5)cc4)c4ccc5c(c4)[C@]4(CCc6ccc(N(c7ccccc7)c7ccccc7)cc64)CC5)cc3c12]

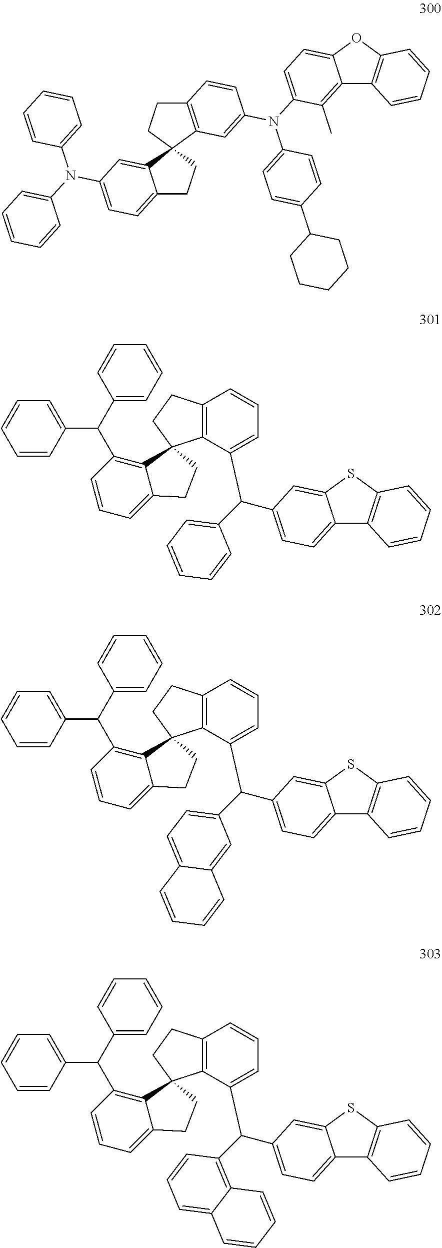 [SMILES: Cc1c(N(c2ccc(C3CCCCC3)cc2)c2ccc3c(c2)[C@]2(CCc4ccc(N(c5ccccc5)c5ccccc5)cc42)CC3)ccc2oc3ccccc3c12 ; c1ccc(C(c2ccccc2)c2cccc3c2[C@@]2(CC3)CCc3cccc(C(c4ccccc4)c4ccc5c(c4)sc4ccccc45)c32)cc1 ; c1ccc(C(c2ccccc2)c2cccc3c2[C@@]2(CC3)CCc3cccc(C(c4ccc5ccccc5c4)c4ccc5c(c4)sc4ccccc45)c32)cc1 ; c1ccc(C(c2ccccc2)c2cccc3c2[C@@]2(CC3)CCc3cccc(C(c4ccc5c(c4)sc4ccccc45)c4cccc5ccccc45)c32)cc1]

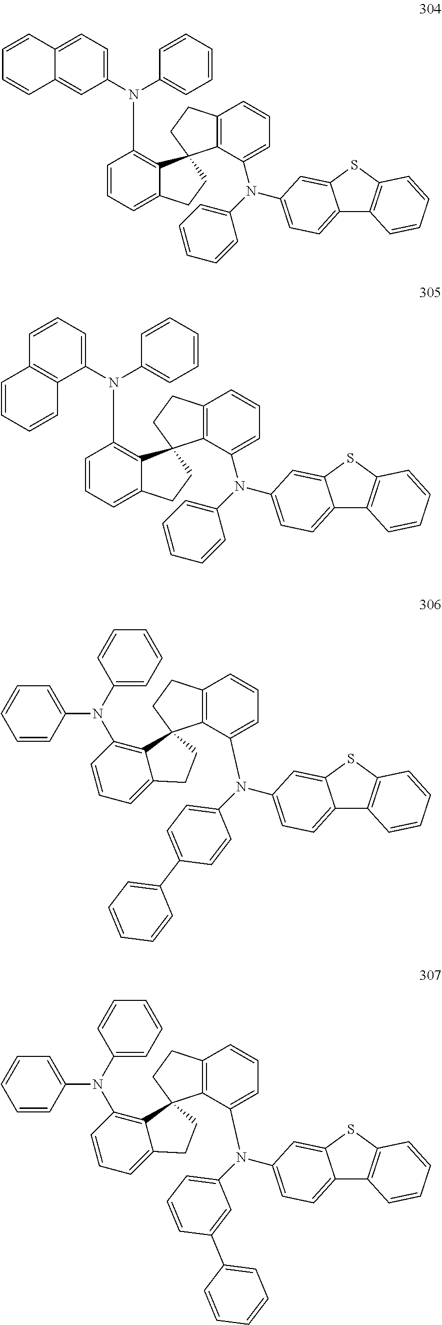 [SMILES: c1ccc(N(c2ccc3ccccc3c2)c2cccc3c2[C@@]2(CC3)CCc3cccc(N(c4ccccc4)c4ccc5c(c4)sc4ccccc45)c32)cc1 ; c1ccc(N(c2ccc3c(c2)sc2ccccc23)c2cccc3c2[C@@]2(CC3)CCc3cccc(N(c4ccccc4)c4cccc5ccccc45)c32)cc1 ; c1ccc(-c2ccc(N(c3ccc4c(c3)sc3ccccc34)c3cccc4c3[C@]3(CCc5cccc(N(c6ccccc6)c6ccccc6)c53)CC4)cc2)cc1 ; c1ccc(-c2cccc(N(c3ccc4c(c3)sc3ccccc34)c3cccc4c3[C@]3(CCc5cccc(N(c6ccccc6)c6ccccc6)c53)CC4)c2)cc1]

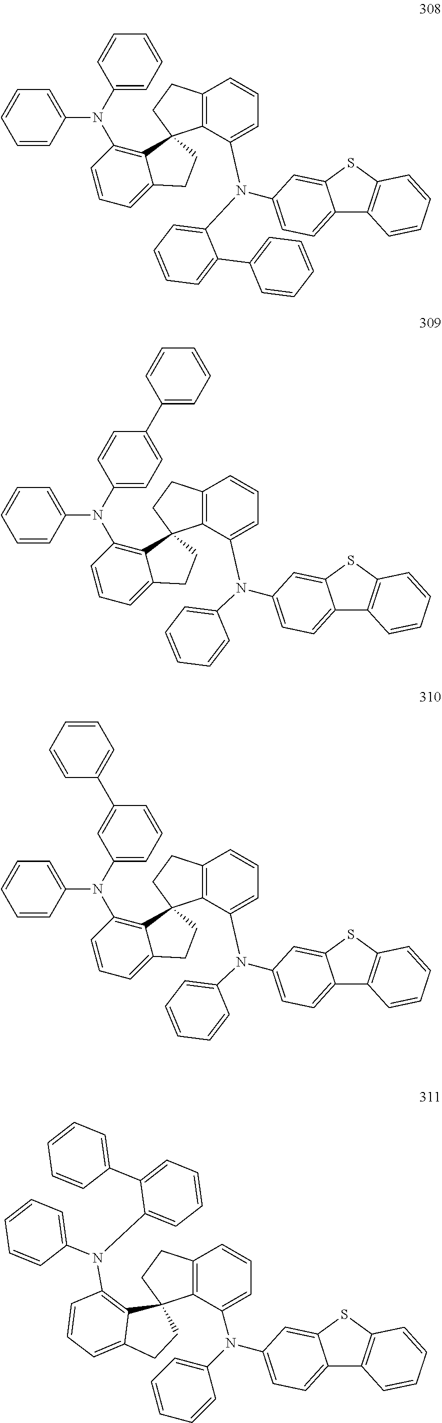 [SMILES: c1ccc(-c2ccccc2N(c2ccc3c(c2)sc2ccccc23)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6c(c5)sc5ccccc56)c43)cc2)cc1 ; c1ccc(-c2cccc(N(c3ccccc3)c3cccc4c3[C@@]3(CC4)CCc4cccc(N(c5ccccc5)c5ccc6c(c5)sc5ccccc56)c43)c2)cc1 ; c1ccc(-c2ccccc2N(c2ccccc2)c2cccc3c2[C@]2(CCc4cccc(N(c5ccccc5)c5ccc6c(c5)sc5ccccc56)c42)CC3)cc1]

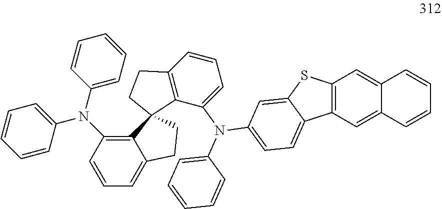 [SMILES: c1ccc(N(c2ccccc2)c2cccc3c2[C@@]2(CC3)CCc3cccc(N(c4ccccc4)c4ccc5c(c4)sc4cc6ccccc6cc45)c32)cc1]

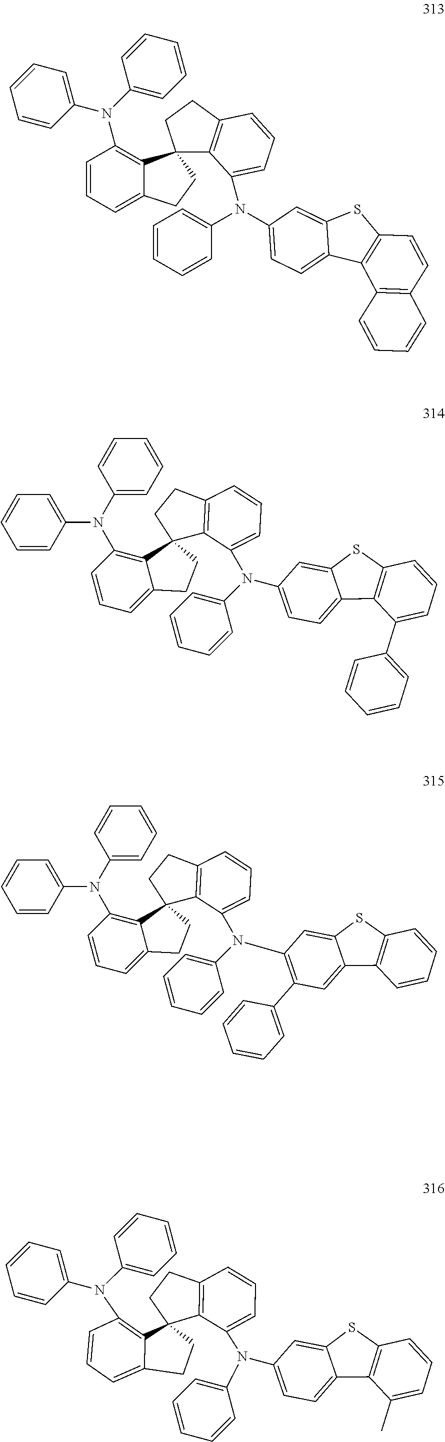 [SMILES: c1ccc(N(c2ccccc2)c2cccc3c2[C@@]2(CC3)CCc3cccc(N(c4ccccc4)c4ccc5c(c4)sc4ccc6ccccc6c45)c32)cc1 ; c1ccc(-c2cccc3sc4cc(N(c5ccccc5)c5cccc6c5[C@]5(CCc7cccc(N(c8ccccc8)c8ccccc8)c75)CC6)ccc4c23)cc1 ; c1ccc(-c2cc3c(cc2N(c2ccccc2)c2cccc4c2[C@]2(CCc5cccc(N(c6ccccc6)c6ccccc6)c52)CC4)sc2ccccc23)cc1 ; Cc1cccc2sc3cc(N(c4ccccc4)c4cccc5c4[C@]4(CCc6cccc(N(c7ccccc7)c7ccccc7)c64)CC5)ccc3c12]

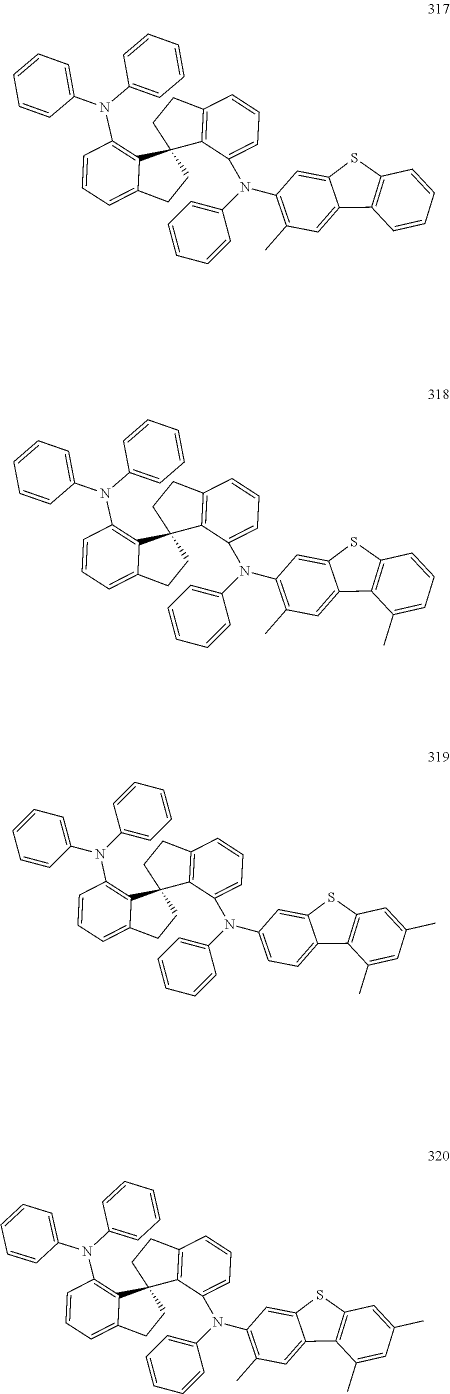 [SMILES: Cc1cc2c(cc1N(c1ccccc1)c1cccc3c1[C@]1(CCc4cccc(N(c5ccccc5)c5ccccc5)c41)CC3)sc1ccccc12 ; Cc1cc2c(cc1N(c1ccccc1)c1cccc3c1[C@]1(CCc4cccc(N(c5ccccc5)c5ccccc5)c41)CC3)sc1cccc(C)c12 ; Cc1cc(C)c2c(c1)sc1cc(N(c3ccccc3)c3cccc4c3[C@]3(CCc5cccc(N(c6ccccc6)c6ccccc6)c53)CC4)ccc12 ; Cc1cc(C)c2c(c1)sc1cc(N(c3ccccc3)c3cccc4c3[C@]3(CCc5cccc(N(c6ccccc6)c6ccccc6)c53)CC4)c(C)cc12]

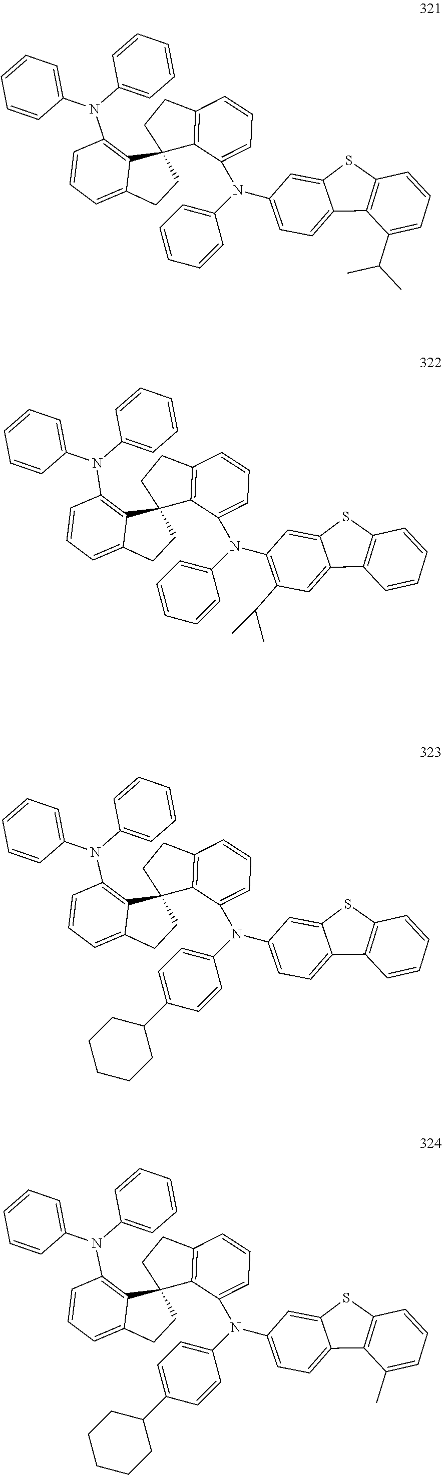 [SMILES: CC(C)c1cccc2sc3cc(N(c4ccccc4)c4cccc5c4[C@]4(CCc6cccc(N(c7ccccc7)c7ccccc7)c64)CC5)ccc3c12 ; CC(C)c1cc2c(cc1N(c1ccccc1)c1cccc3c1[C@]1(CCc4cccc(N(c5ccccc5)c5ccccc5)c41)CC3)sc1ccccc12 ; c1ccc(N(c2ccccc2)c2cccc3c2[C@@]2(CC3)CCc3cccc(N(c4ccc(C5CCCCC5)cc4)c4ccc5c(c4)sc4ccccc45)c32)cc1 ; Cc1cccc2sc3cc(N(c4ccc(C5CCCCC5)cc4)c4cccc5c4[C@]4(CCc6cccc(N(c7ccccc7)c7ccccc7)c64)CC5)ccc3c12]

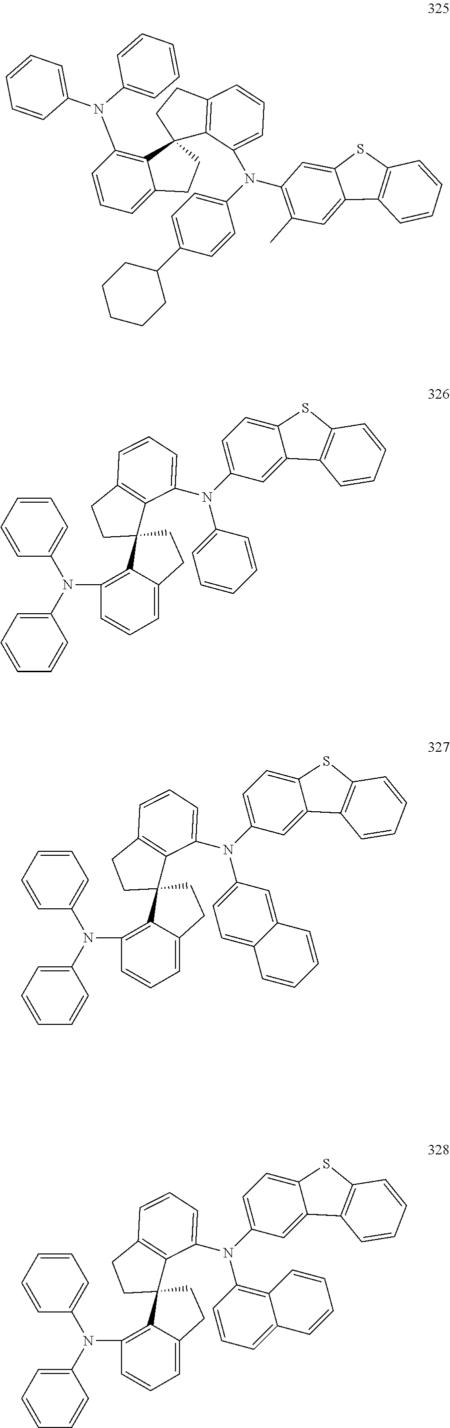 [SMILES: Cc1cc2c(cc1N(c1ccc(C3CCCCC3)cc1)c1cccc3c1[C@]1(CCc4cccc(N(c5ccccc5)c5ccccc5)c41)CC3)sc1ccccc12 ; c1ccc(N(c2ccccc2)c2cccc3c2[C@@]2(CC3)CCc3cccc(N(c4ccccc4)c4ccc5sc6ccccc6c5c4)c32)cc1 ; c1ccc(N(c2ccccc2)c2cccc3c2[C@@]2(CC3)CCc3cccc(N(c4ccc5ccccc5c4)c4ccc5sc6ccccc6c5c4)c32)cc1 ; c1ccc(N(c2ccccc2)c2cccc3c2[C@@]2(CC3)CCc3cccc(N(c4ccc5sc6ccccc6c5c4)c4cccc5ccccc45)c32)cc1]

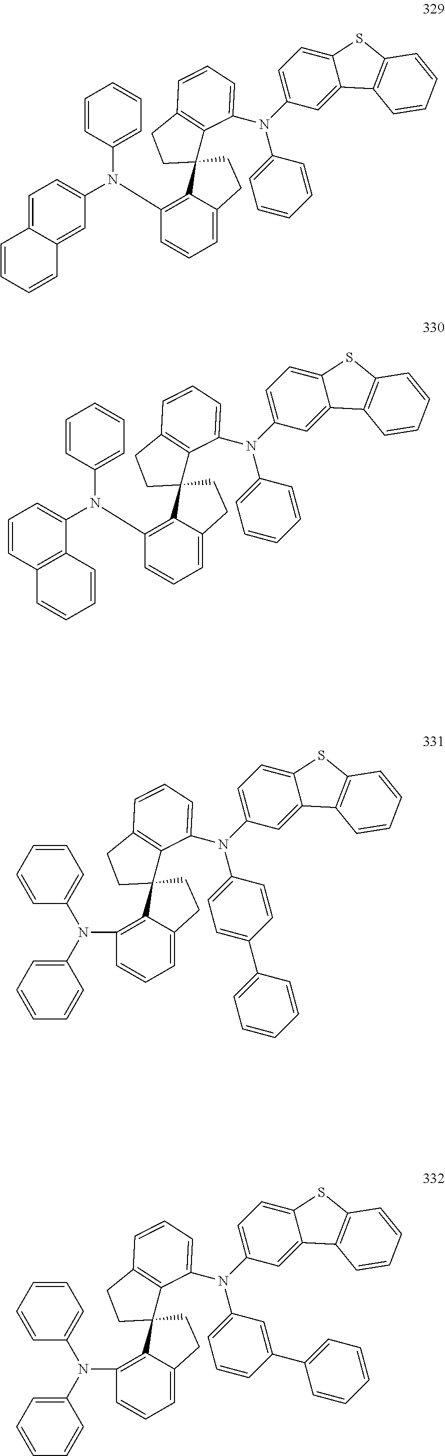 [SMILES: c1ccc(N(c2ccc3ccccc3c2)c2cccc3c2[C@@]2(CC3)CCc3cccc(N(c4ccccc4)c4ccc5sc6ccccc6c5c4)c32)cc1 ; c1ccc(N(c2ccc3sc4ccccc4c3c2)c2cccc3c2[C@@]2(CC3)CCc3cccc(N(c4ccccc4)c4cccc5ccccc45)c32)cc1 ; c1ccc(-c2ccc(N(c3ccc4sc5ccccc5c4c3)c3cccc4c3[C@]3(CCc5cccc(N(c6ccccc6)c6ccccc6)c53)CC4)cc2)cc1 ; c1ccc(-c2cccc(N(c3ccc4sc5ccccc5c4c3)c3cccc4c3[C@]3(CCc5cccc(N(c6ccccc6)c6ccccc6)c53)CC4)c2)cc1]

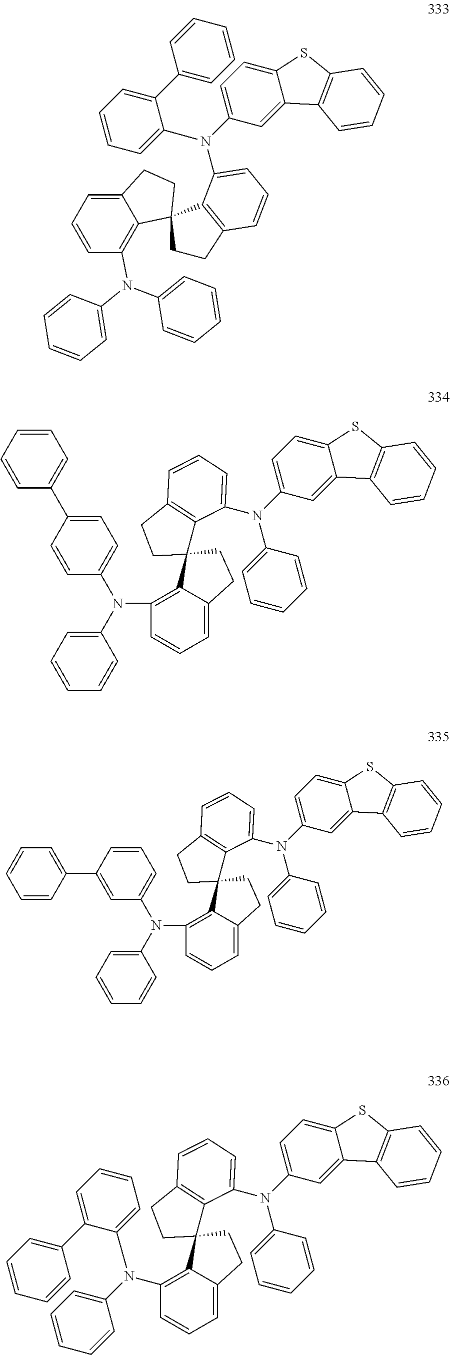 [SMILES: c1ccc(-c2ccccc2N(c2ccc3sc4ccccc4c3c2)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6sc7ccccc7c6c5)c43)cc2)cc1 ; c1ccc(-c2cccc(N(c3ccccc3)c3cccc4c3[C@@]3(CC4)CCc4cccc(N(c5ccccc5)c5ccc6sc7ccccc7c6c5)c43)c2)cc1 ; c1ccc(-c2ccccc2N(c2ccccc2)c2cccc3c2[C@]2(CCc4cccc(N(c5ccccc5)c5ccc6sc7ccccc7c6c5)c42)CC3)cc1]

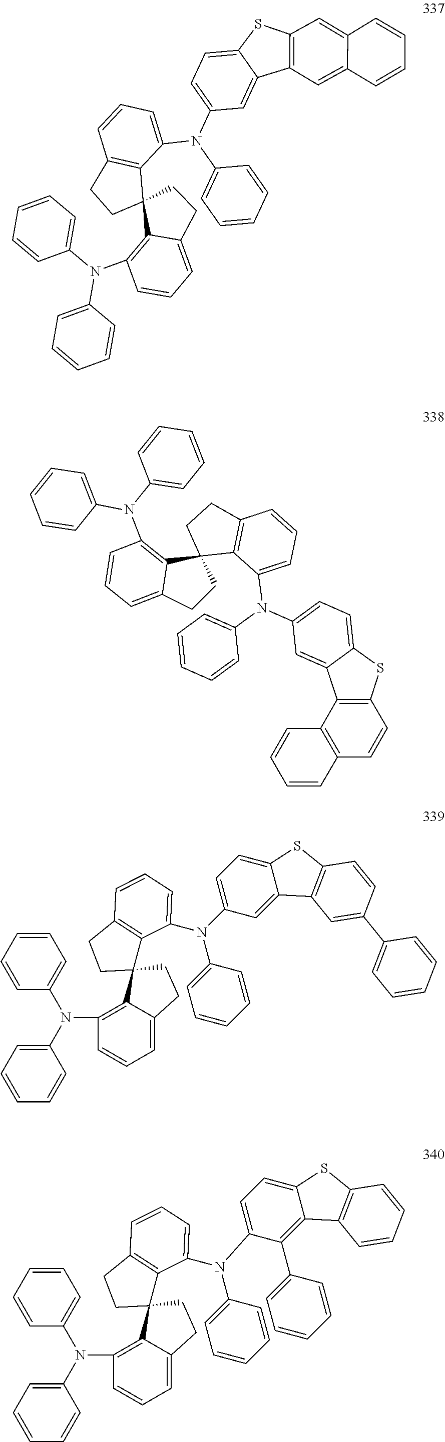 [SMILES: c1ccc(N(c2ccccc2)c2cccc3c2[C@@]2(CC3)CCc3cccc(N(c4ccccc4)c4ccc5sc6cc7ccccc7cc6c5c4)c32)cc1 ; c1ccc(N(c2ccccc2)c2cccc3c2[C@@]2(CC3)CCc3cccc(N(c4ccccc4)c4ccc5sc6ccc7ccccc7c6c5c4)c32)cc1 ; c1ccc(-c2ccc3sc4ccc(N(c5ccccc5)c5cccc6c5[C@]5(CCc7cccc(N(c8ccccc8)c8ccccc8)c75)CC6)cc4c3c2)cc1 ; c1ccc(-c2c(N(c3ccccc3)c3cccc4c3[C@]3(CCc5cccc(N(c6ccccc6)c6ccccc6)c53)CC4)ccc3sc4ccccc4c23)cc1]

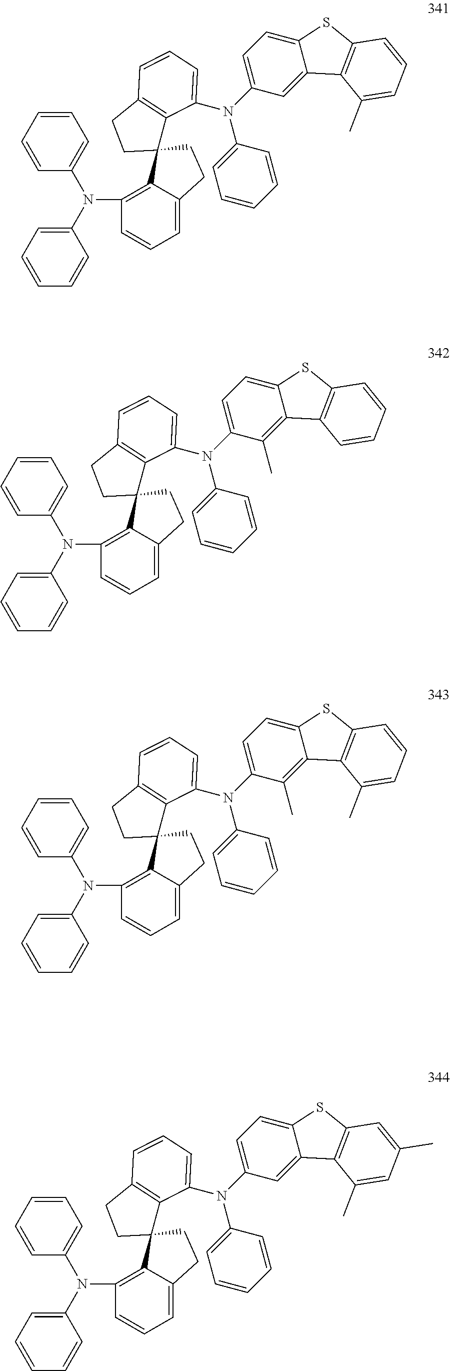 [SMILES: Cc1cccc2sc3ccc(N(c4ccccc4)c4cccc5c4[C@]4(CCc6cccc(N(c7ccccc7)c7ccccc7)c64)CC5)cc3c12 ; Cc1c(N(c2ccccc2)c2cccc3c2[C@]2(CCc4cccc(N(c5ccccc5)c5ccccc5)c42)CC3)ccc2sc3ccccc3c12 ; Cc1cccc2sc3ccc(N(c4ccccc4)c4cccc5c4[C@]4(CCc6cccc(N(c7ccccc7)c7ccccc7)c64)CC5)c(C)c3c12 ; Cc1cc(C)c2c(c1)sc1ccc(N(c3ccccc3)c3cccc4c3[C@]3(CCc5cccc(N(c6ccccc6)c6ccccc6)c53)CC4)cc12]

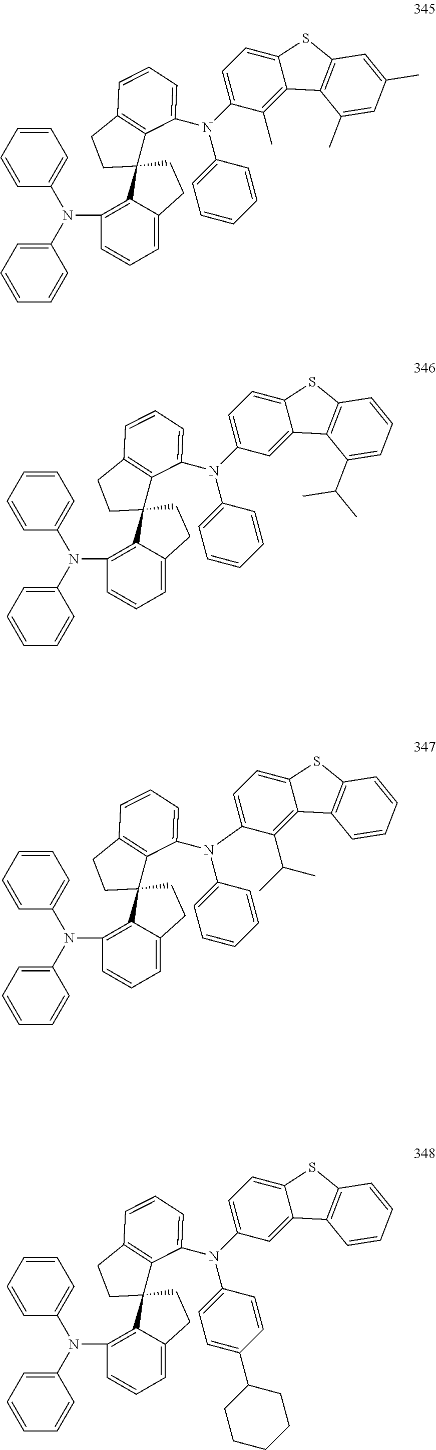 [SMILES: Cc1cc(C)c2c(c1)sc1ccc(N(c3ccccc3)c3cccc4c3[C@]3(CCc5cccc(N(c6ccccc6)c6ccccc6)c53)CC4)c(C)c12 ; CC(C)c1cccc2sc3ccc(N(c4ccccc4)c4cccc5c4[C@]4(CCc6cccc(N(c7ccccc7)c7ccccc7)c64)CC5)cc3c12 ; CC(C)c1c(N(c2ccccc2)c2cccc3c2[C@]2(CCc4cccc(N(c5ccccc5)c5ccccc5)c42)CC3)ccc2sc3ccccc3c12 ; c1ccc(N(c2ccccc2)c2cccc3c2[C@@]2(CC3)CCc3cccc(N(c4ccc(C5CCCCC5)cc4)c4ccc5sc6ccccc6c5c4)c32)cc1]

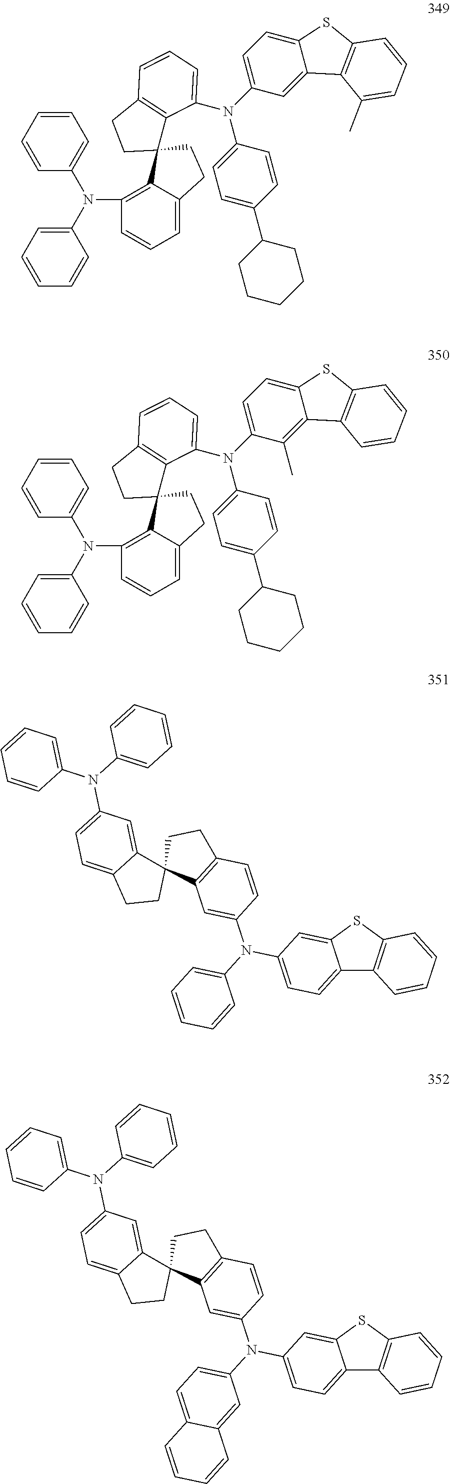 [SMILES: Cc1cccc2sc3ccc(N(c4ccc(C5CCCCC5)cc4)c4cccc5c4[C@]4(CCc6cccc(N(c7ccccc7)c7ccccc7)c64)CC5)cc3c12 ; Cc1c(N(c2ccc(C3CCCCC3)cc2)c2cccc3c2[C@]2(CCc4cccc(N(c5ccccc5)c5ccccc5)c42)CC3)ccc2sc3ccccc3c12 ; c1ccc(N(c2ccccc2)c2ccc3c(c2)[C@@]2(CC3)CCc3ccc(N(c4ccccc4)c4ccc5c(c4)sc4ccccc45)cc32)cc1 ; c1ccc(N(c2ccccc2)c2ccc3c(c2)[C@@]2(CC3)CCc3ccc(N(c4ccc5ccccc5c4)c4ccc5c(c4)sc4ccccc45)cc32)cc1]

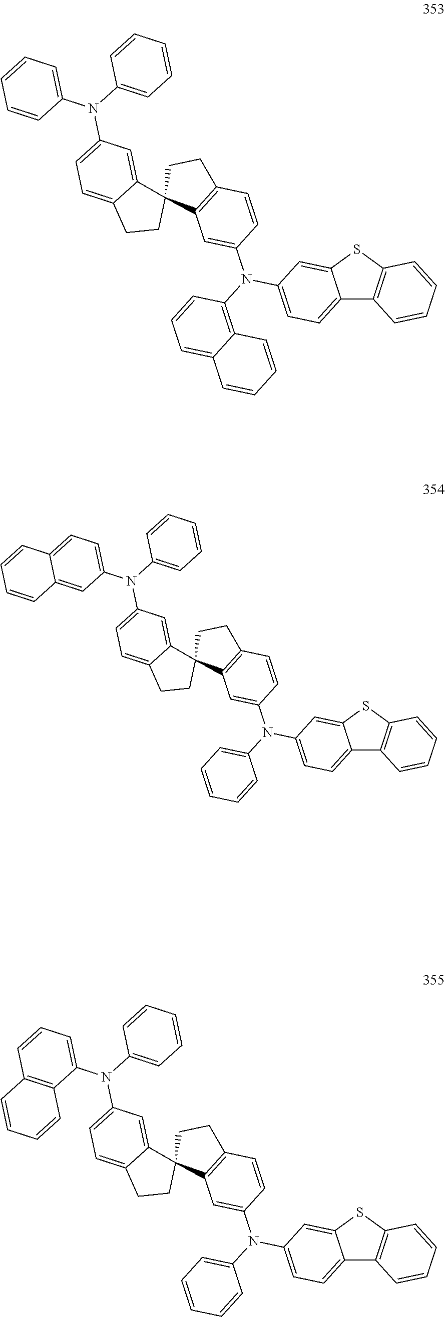 [SMILES: c1ccc(N(c2ccccc2)c2ccc3c(c2)[C@@]2(CC3)CCc3ccc(N(c4ccc5c(c4)sc4ccccc45)c4cccc5ccccc45)cc32)cc1 ; c1ccc(N(c2ccc3c(c2)[C@@]2(CC3)CCc3ccc(N(c4ccccc4)c4ccc5c(c4)sc4ccccc45)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)sc2ccccc23)cc1]

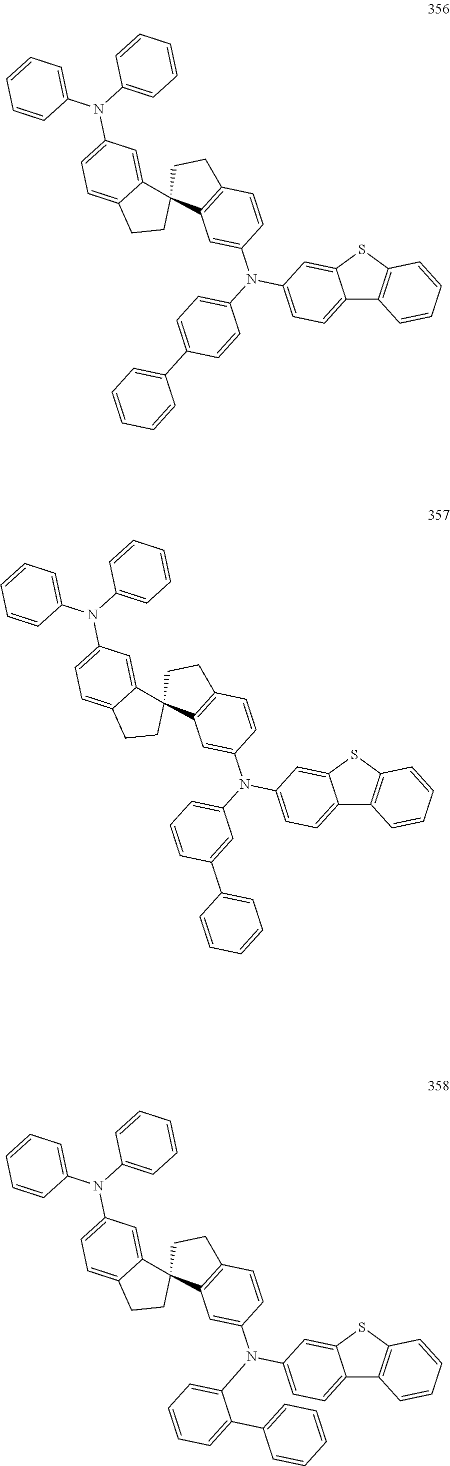 [SMILES: c1ccc(-c2ccc(N(c3ccc4c(c3)[C@]3(CCc5ccc(N(c6ccccc6)c6ccccc6)cc53)CC4)c3ccc4c(c3)sc3ccccc34)cc2)cc1 ; c1ccc(-c2cccc(N(c3ccc4c(c3)[C@]3(CCc5ccc(N(c6ccccc6)c6ccccc6)cc53)CC4)c3ccc4c(c3)sc3ccccc34)c2)cc1 ; c1ccc(-c2ccccc2N(c2ccc3c(c2)[C@]2(CCc4ccc(N(c5ccccc5)c5ccccc5)cc42)CC3)c2ccc3c(c2)sc2ccccc23)cc1]

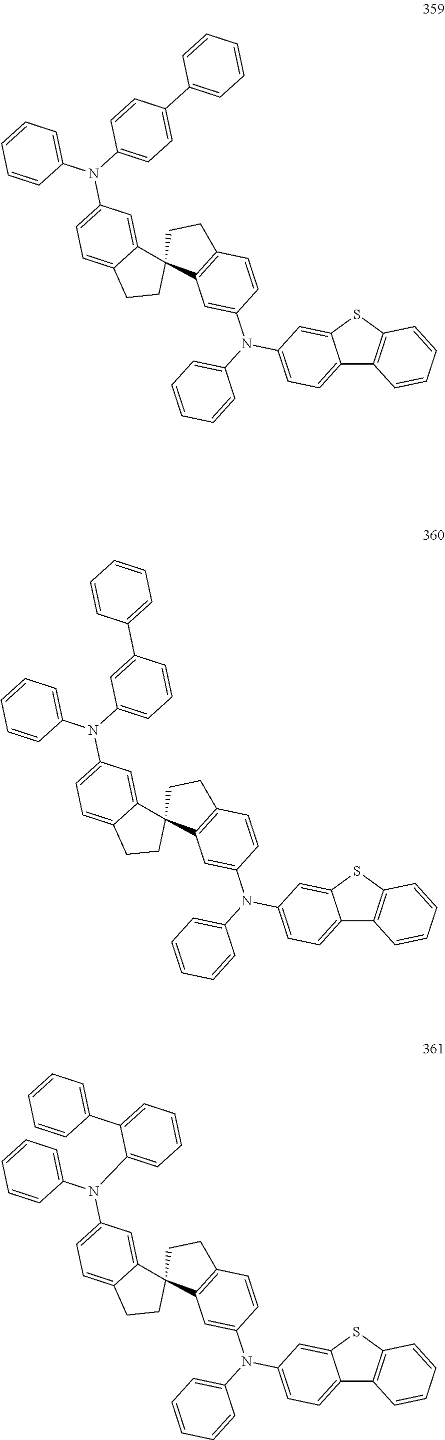 [SMILES: c1ccc(-c2ccc(N(c3ccccc3)c3ccc4c(c3)[C@@]3(CC4)CCc4ccc(N(c5ccccc5)c5ccc6c(c5)sc5ccccc56)cc43)cc2)cc1 ; c1ccc(-c2cccc(N(c3ccccc3)c3ccc4c(c3)[C@@]3(CC4)CCc4ccc(N(c5ccccc5)c5ccc6c(c5)sc5ccccc56)cc43)c2)cc1 ; c1ccc(-c2ccccc2N(c2ccccc2)c2ccc3c(c2)[C@]2(CCc4ccc(N(c5ccccc5)c5ccc6c(c5)sc5ccccc56)cc42)CC3)cc1]

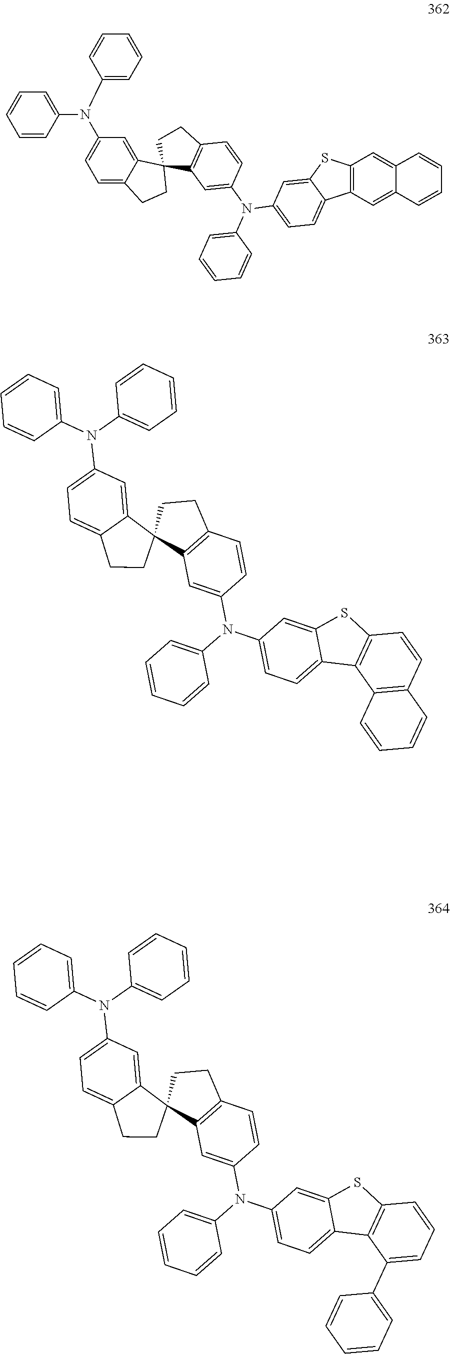 [SMILES: c1ccc(N(c2ccccc2)c2ccc3c(c2)[C@@]2(CC3)CCc3ccc(N(c4ccccc4)c4ccc5c(c4)sc4cc6ccccc6cc45)cc32)cc1 ; c1ccc(N(c2ccccc2)c2ccc3c(c2)[C@@]2(CC3)CCc3ccc(N(c4ccccc4)c4ccc5c(c4)sc4ccc6ccccc6c45)cc32)cc1 ; c1ccc(-c2cccc3sc4cc(N(c5ccccc5)c5ccc6c(c5)[C@]5(CCc7ccc(N(c8ccccc8)c8ccccc8)cc75)CC6)ccc4c23)cc1]

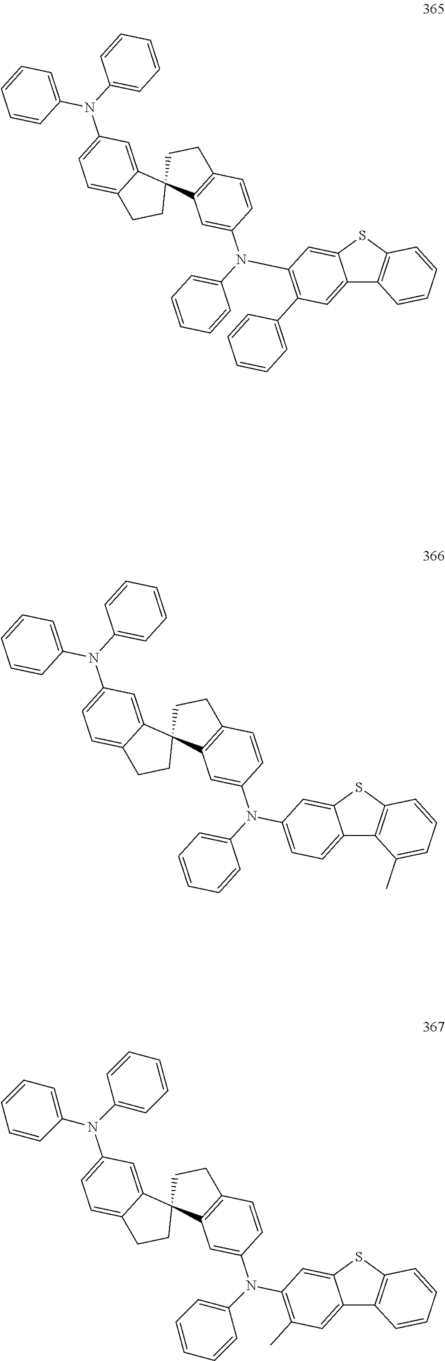 [SMILES: c1ccc(-c2cc3c(cc2N(c2ccccc2)c2ccc4c(c2)[C@]2(CCc5ccc(N(c6ccccc6)c6ccccc6)cc52)CC4)sc2ccccc23)cc1 ; Cc1cccc2sc3cc(N(c4ccccc4)c4ccc5c(c4)[C@]4(CCc6ccc(N(c7ccccc7)c7ccccc7)cc64)CC5)ccc3c12 ; Cc1cc2c(cc1N(c1ccccc1)c1ccc3c(c1)[C@]1(CCc4ccc(N(c5ccccc5)c5ccccc5)cc41)CC3)sc1ccccc12]

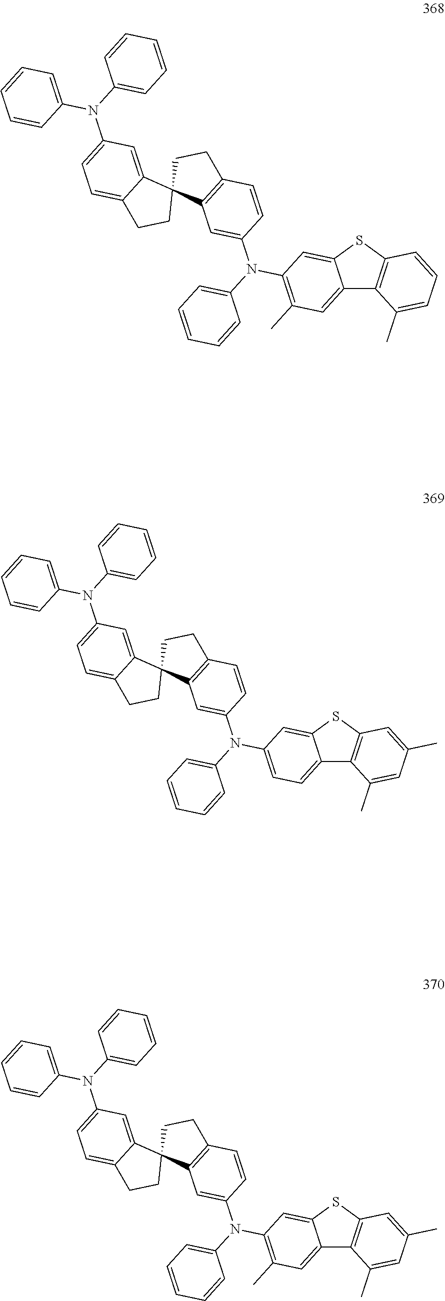 [SMILES: Cc1cc2c(cc1N(c1ccccc1)c1ccc3c(c1)[C@]1(CCc4ccc(N(c5ccccc5)c5ccccc5)cc41)CC3)sc1cccc(C)c12 ; Cc1cc(C)c2c(c1)sc1cc(N(c3ccccc3)c3ccc4c(c3)[C@]3(CCc5ccc(N(c6ccccc6)c6ccccc6)cc53)CC4)ccc12 ; Cc1cc(C)c2c(c1)sc1cc(N(c3ccccc3)c3ccc4c(c3)[C@]3(CCc5ccc(N(c6ccccc6)c6ccccc6)cc53)CC4)c(C)cc12]

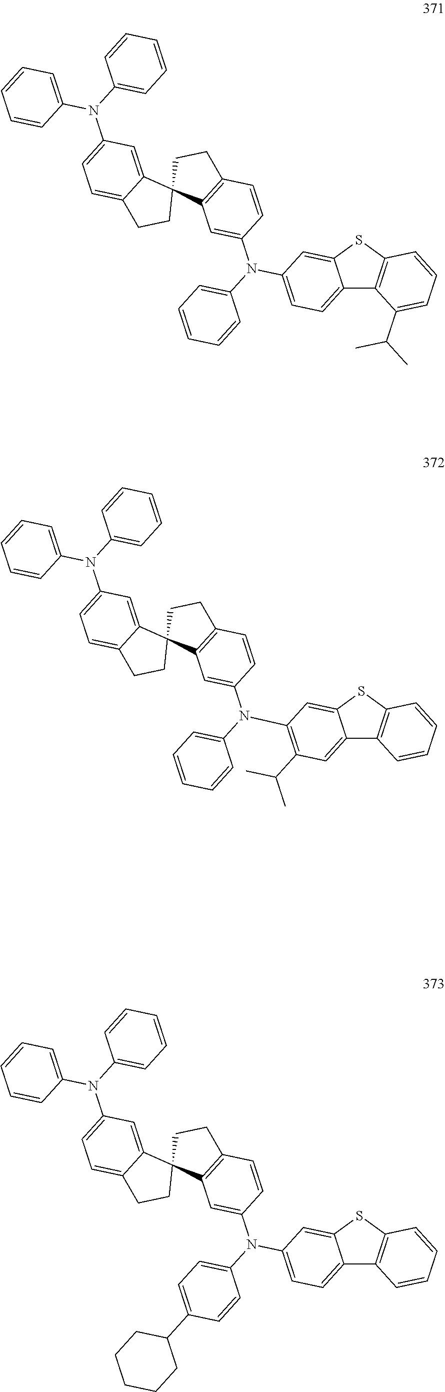 [SMILES: CC(C)c1cccc2sc3cc(N(c4ccccc4)c4ccc5c(c4)[C@]4(CCc6ccc(N(c7ccccc7)c7ccccc7)cc64)CC5)ccc3c12 ; CC(C)c1cc2c(cc1N(c1ccccc1)c1ccc3c(c1)[C@]1(CCc4ccc(N(c5ccccc5)c5ccccc5)cc41)CC3)sc1ccccc12 ; c1ccc(N(c2ccccc2)c2ccc3c(c2)[C@@]2(CC3)CCc3ccc(N(c4ccc(C5CCCCC5)cc4)c4ccc5c(c4)sc4ccccc45)cc32)cc1]

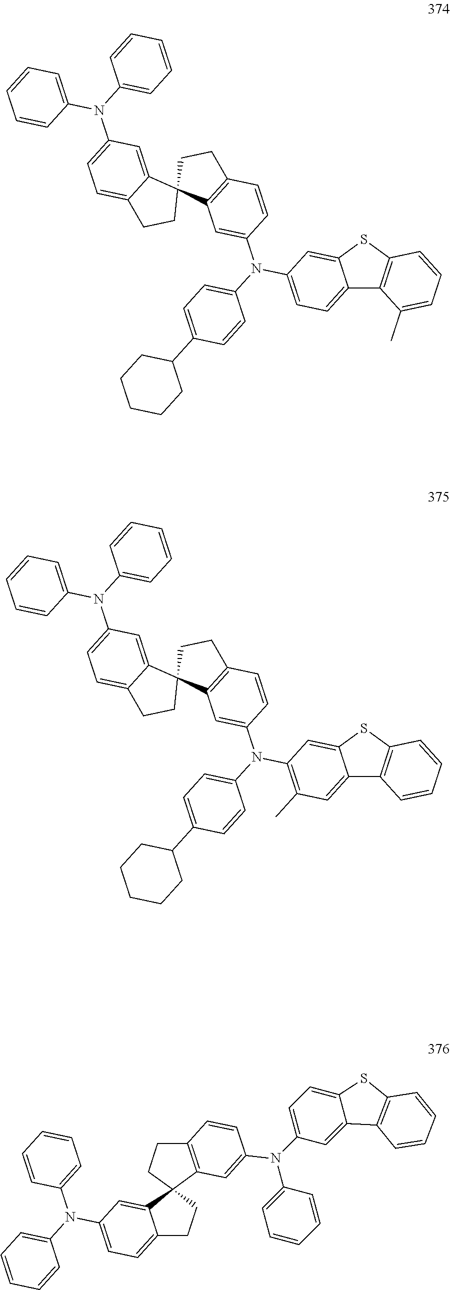 [SMILES: Cc1cccc2sc3cc(N(c4ccc(C5CCCCC5)cc4)c4ccc5c(c4)[C@]4(CCc6ccc(N(c7ccccc7)c7ccccc7)cc64)CC5)ccc3c12 ; Cc1cc2c(cc1N(c1ccc(C3CCCCC3)cc1)c1ccc3c(c1)[C@]1(CCc4ccc(N(c5ccccc5)c5ccccc5)cc41)CC3)sc1ccccc12 ; c1ccc(N(c2ccccc2)c2ccc3c(c2)[C@@]2(CC3)CCc3ccc(N(c4ccccc4)c4ccc5sc6ccccc6c5c4)cc32)cc1]

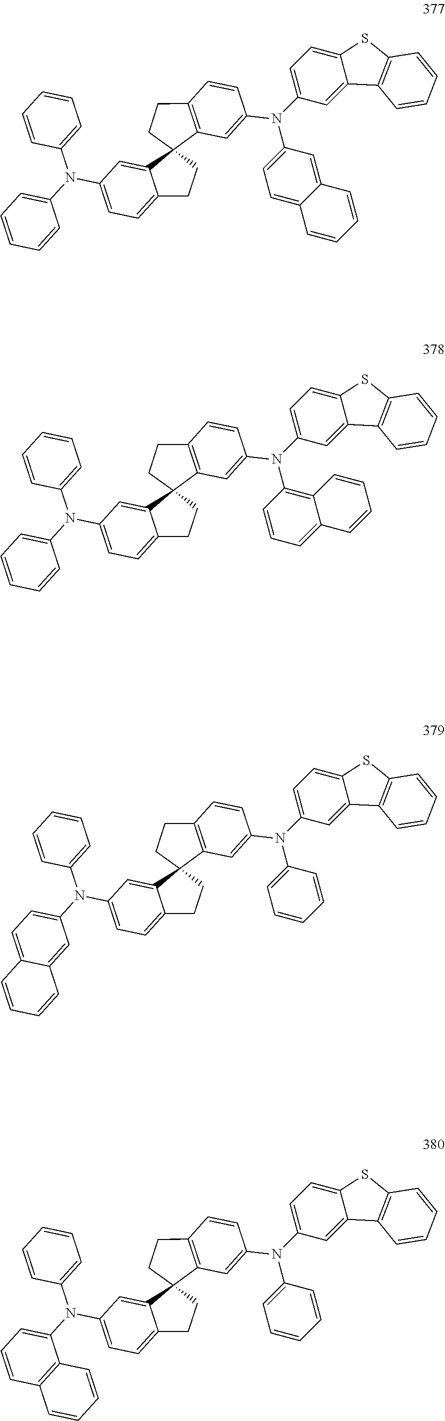 [SMILES: c1ccc(N(c2ccccc2)c2ccc3c(c2)[C@@]2(CC3)CCc3ccc(N(c4ccc5ccccc5c4)c4ccc5sc6ccccc6c5c4)cc32)cc1 ; c1ccc(N(c2ccccc2)c2ccc3c(c2)[C@@]2(CC3)CCc3ccc(N(c4ccc5sc6ccccc6c5c4)c4cccc5ccccc45)cc32)cc1 ; c1ccc(N(c2ccc3c(c2)[C@@]2(CC3)CCc3ccc(N(c4ccccc4)c4ccc5sc6ccccc6c5c4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3sc4ccccc4c3c2)cc1]

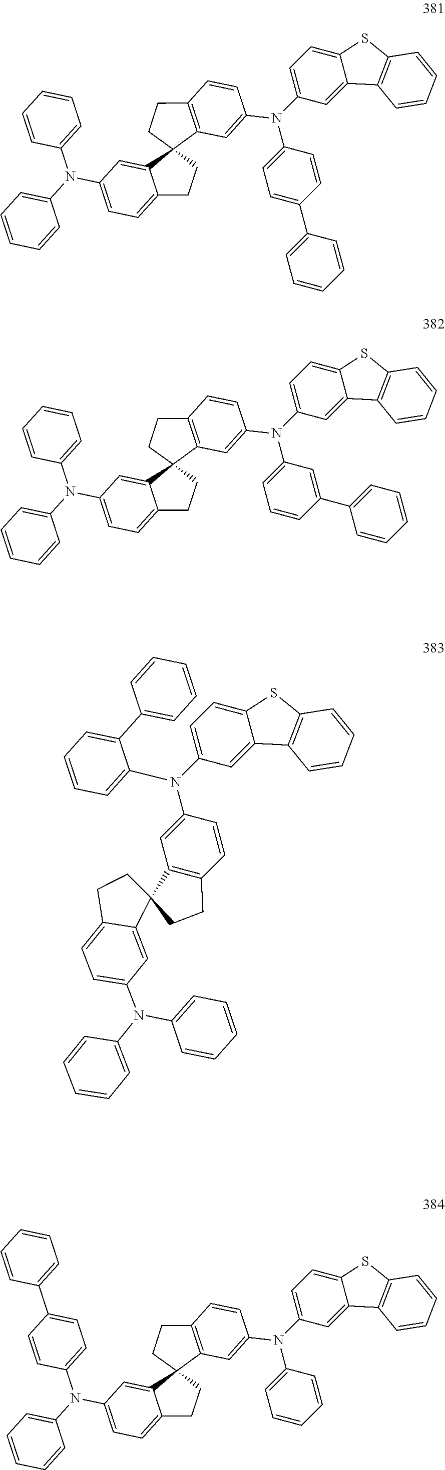 [SMILES: c1ccc(-c2ccc(N(c3ccc4c(c3)[C@]3(CCc5ccc(N(c6ccccc6)c6ccccc6)cc53)CC4)c3ccc4sc5ccccc5c4c3)cc2)cc1 ; c1ccc(-c2cccc(N(c3ccc4c(c3)[C@]3(CCc5ccc(N(c6ccccc6)c6ccccc6)cc53)CC4)c3ccc4sc5ccccc5c4c3)c2)cc1 ; c1ccc(-c2ccccc2N(c2ccc3c(c2)[C@]2(CCc4ccc(N(c5ccccc5)c5ccccc5)cc42)CC3)c2ccc3sc4ccccc4c3c2)cc1 ; c1ccc(-c2ccc(N(c3ccccc3)c3ccc4c(c3)[C@@]3(CC4)CCc4ccc(N(c5ccccc5)c5ccc6sc7ccccc7c6c5)cc43)cc2)cc1]

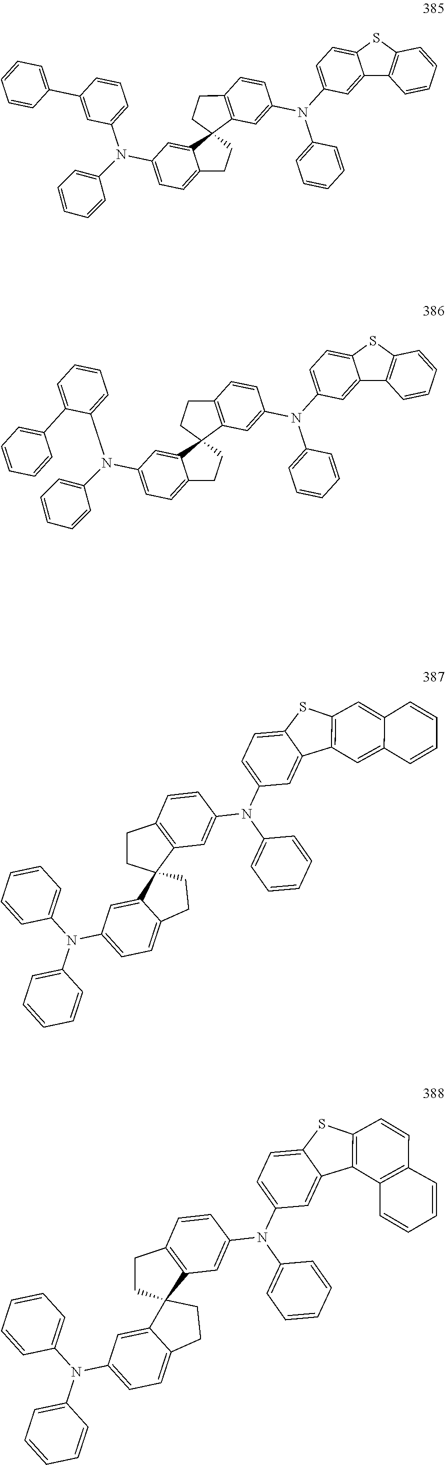 [SMILES: c1ccc(-c2cccc(N(c3ccccc3)c3ccc4c(c3)[C@@]3(CC4)CCc4ccc(N(c5ccccc5)c5ccc6sc7ccccc7c6c5)cc43)c2)cc1 ; c1ccc(-c2ccccc2N(c2ccccc2)c2ccc3c(c2)[C@]2(CCc4ccc(N(c5ccccc5)c5ccc6sc7ccccc7c6c5)cc42)CC3)cc1 ; c1ccc(N(c2ccccc2)c2ccc3c(c2)[C@@]2(CC3)CCc3ccc(N(c4ccccc4)c4ccc5sc6cc7ccccc7cc6c5c4)cc32)cc1 ; c1ccc(N(c2ccccc2)c2ccc3c(c2)[C@@]2(CC3)CCc3ccc(N(c4ccccc4)c4ccc5sc6ccc7ccccc7c6c5c4)cc32)cc1]

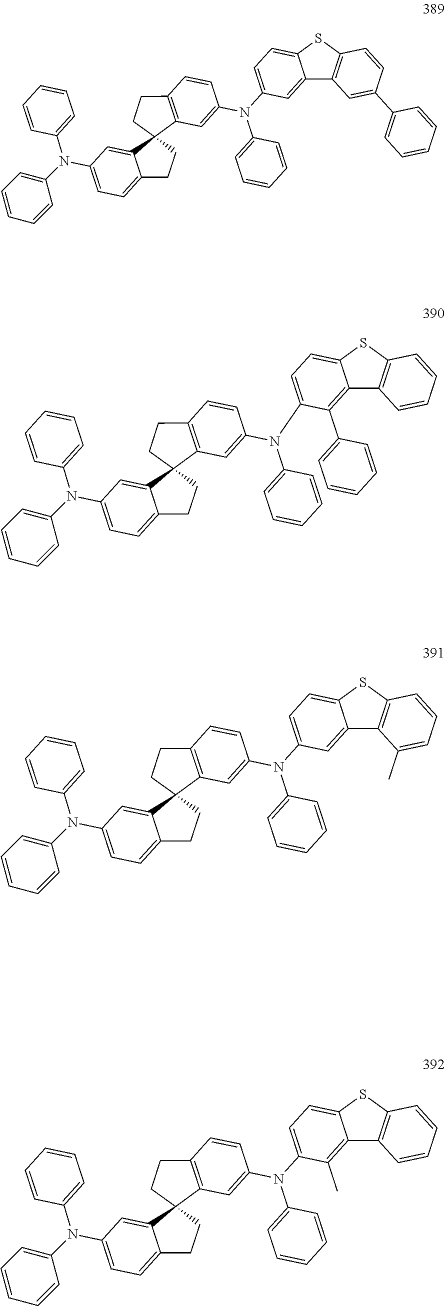 [SMILES: c1ccc(-c2ccc3sc4ccc(N(c5ccccc5)c5ccc6c(c5)[C@]5(CCc7ccc(N(c8ccccc8)c8ccccc8)cc75)CC6)cc4c3c2)cc1 ; c1ccc(-c2c(N(c3ccccc3)c3ccc4c(c3)[C@]3(CCc5ccc(N(c6ccccc6)c6ccccc6)cc53)CC4)ccc3sc4ccccc4c23)cc1 ; Cc1cccc2sc3ccc(N(c4ccccc4)c4ccc5c(c4)[C@]4(CCc6ccc(N(c7ccccc7)c7ccccc7)cc64)CC5)cc3c12 ; Cc1c(N(c2ccccc2)c2ccc3c(c2)[C@]2(CCc4ccc(N(c5ccccc5)c5ccccc5)cc42)CC3)ccc2sc3ccccc3c12]

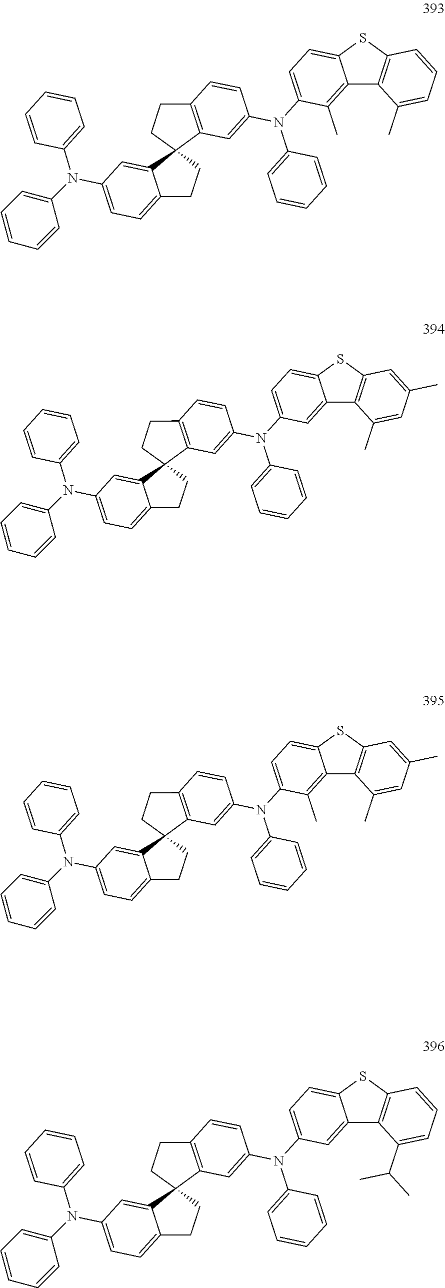 [SMILES: Cc1cccc2sc3ccc(N(c4ccccc4)c4ccc5c(c4)[C@]4(CCc6ccc(N(c7ccccc7)c7ccccc7)cc64)CC5)c(C)c3c12 ; Cc1cc(C)c2c(c1)sc1ccc(N(c3ccccc3)c3ccc4c(c3)[C@]3(CCc5ccc(N(c6ccccc6)c6ccccc6)cc53)CC4)cc12 ; Cc1cc(C)c2c(c1)sc1ccc(N(c3ccccc3)c3ccc4c(c3)[C@]3(CCc5ccc(N(c6ccccc6)c6ccccc6)cc53)CC4)c(C)c12 ; CC(C)c1cccc2sc3ccc(N(c4ccccc4)c4ccc5c(c4)[C@]4(CCc6ccc(N(c7ccccc7)c7ccccc7)cc64)CC5)cc3c12]

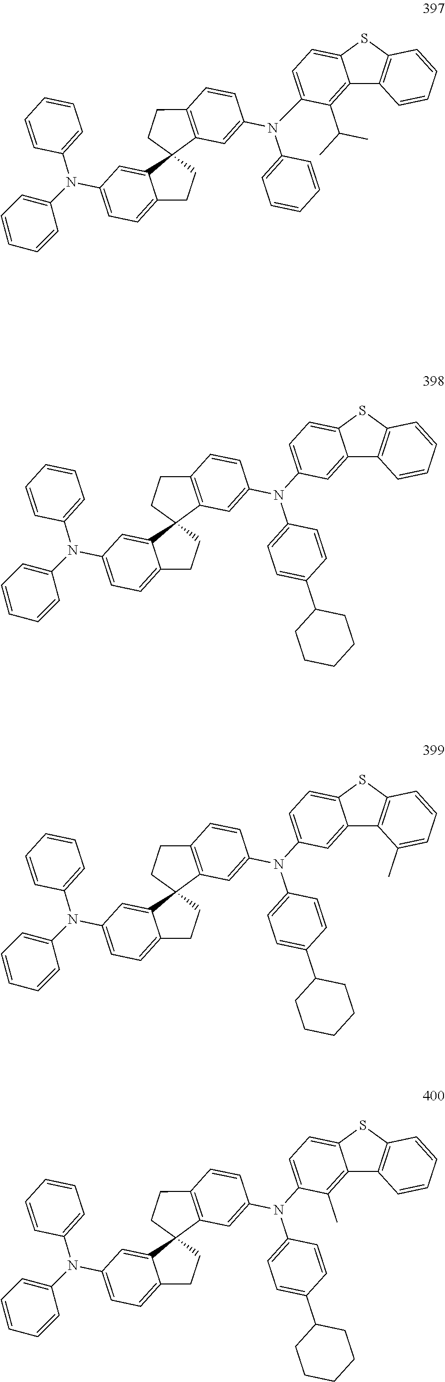 [SMILES: CC(C)c1c(N(c2ccccc2)c2ccc3c(c2)[C@]2(CCc4ccc(N(c5ccccc5)c5ccccc5)cc42)CC3)ccc2sc3ccccc3c12 ; c1ccc(N(c2ccccc2)c2ccc3c(c2)[C@@]2(CC3)CCc3ccc(N(c4ccc(C5CCCCC5)cc4)c4ccc5sc6ccccc6c5c4)cc32)cc1 ; Cc1cccc2sc3ccc(N(c4ccc(C5CCCCC5)cc4)c4ccc5c(c4)[C@]4(CCc6ccc(N(c7ccccc7)c7ccccc7)cc64)CC5)cc3c12 ; Cc1c(N(c2ccc(C3CCCCC3)cc2)c2ccc3c(c2)[C@]2(CCc4ccc(N(c5ccccc5)c5ccccc5)cc42)CC3)ccc2sc3ccccc3c12]

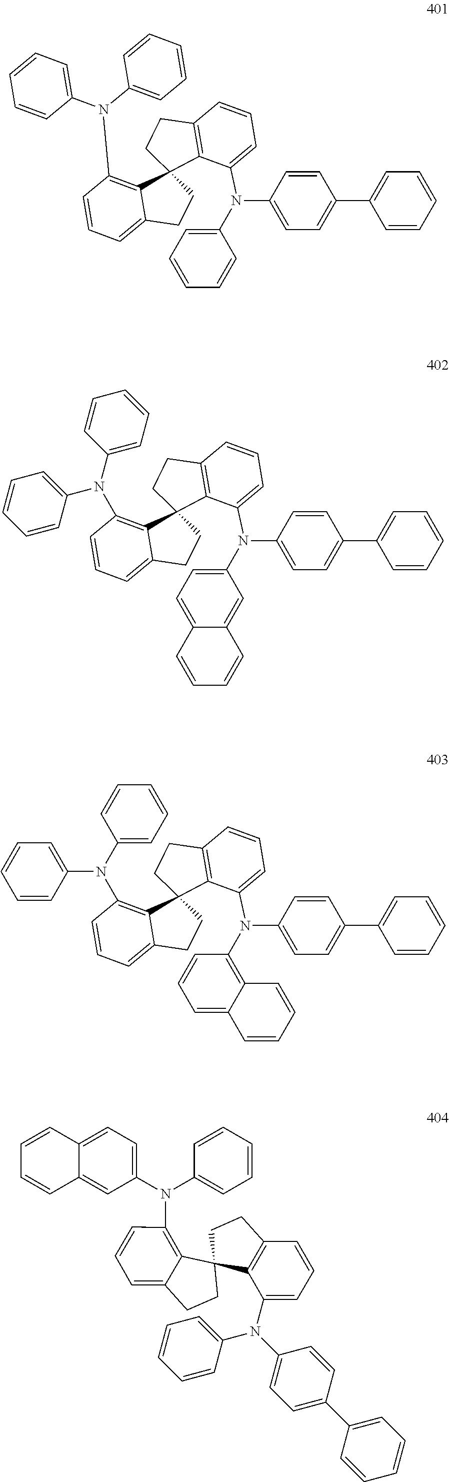 [SMILES: c1ccc(-c2ccc(N(c3ccccc3)c3cccc4c3[C@]3(CCc5cccc(N(c6ccccc6)c6ccccc6)c53)CC4)cc2)cc1 ; c1ccc(-c2ccc(N(c3ccc4ccccc4c3)c3cccc4c3[C@]3(CCc5cccc(N(c6ccccc6)c6ccccc6)c53)CC4)cc2)cc1 ; c1ccc(-c2ccc(N(c3cccc4c3[C@]3(CCc5cccc(N(c6ccccc6)c6ccccc6)c53)CC4)c3cccc4ccccc34)cc2)cc1 ; c1ccc(-c2ccc(N(c3ccccc3)c3cccc4c3[C@@]3(CC4)CCc4cccc(N(c5ccccc5)c5ccc6ccccc6c5)c43)cc2)cc1]

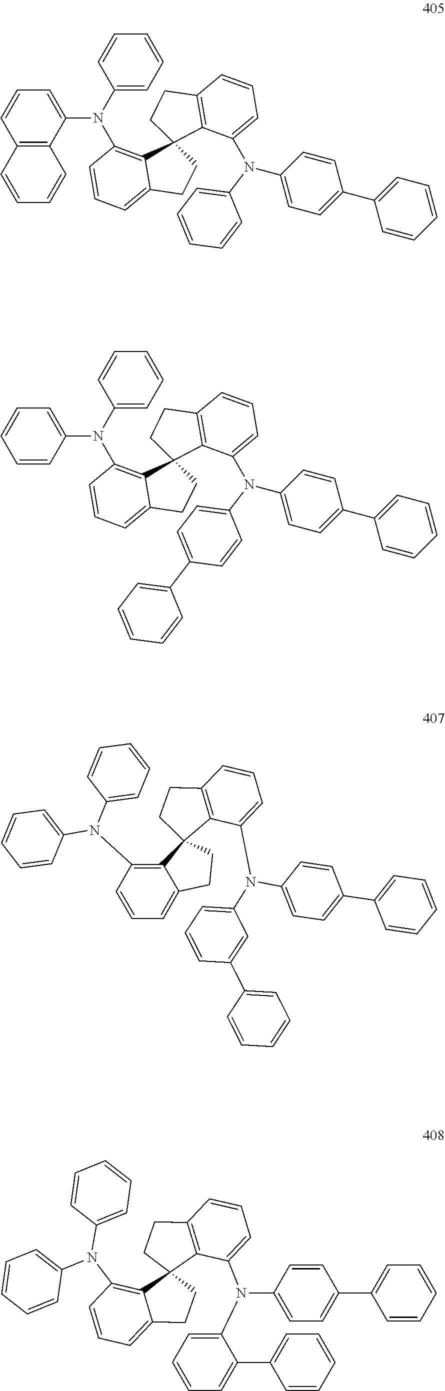 [SMILES: c1ccc(-c2ccc(N(c3ccccc3)c3cccc4c3[C@@]3(CC4)CCc4cccc(N(c5ccccc5)c5cccc6ccccc56)c43)cc2)cc1 ; c1ccc(-c2ccc(N(c3ccc(-c4ccccc4)cc3)c3cccc4c3[C@]3(CCc5cccc(N(c6ccccc6)c6ccccc6)c53)CC4)cc2)cc1 ; c1ccc(-c2ccc(N(c3cccc(-c4ccccc4)c3)c3cccc4c3[C@]3(CCc5cccc(N(c6ccccc6)c6ccccc6)c53)CC4)cc2)cc1 ; c1ccc(-c2ccc(N(c3ccccc3-c3ccccc3)c3cccc4c3[C@]3(CCc5cccc(N(c6ccccc6)c6ccccc6)c53)CC4)cc2)cc1]

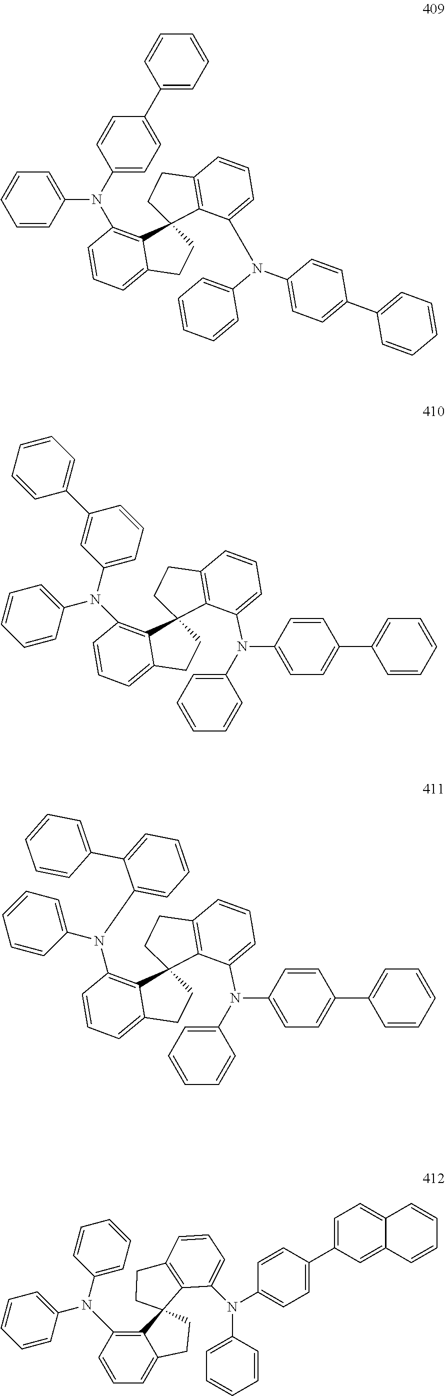

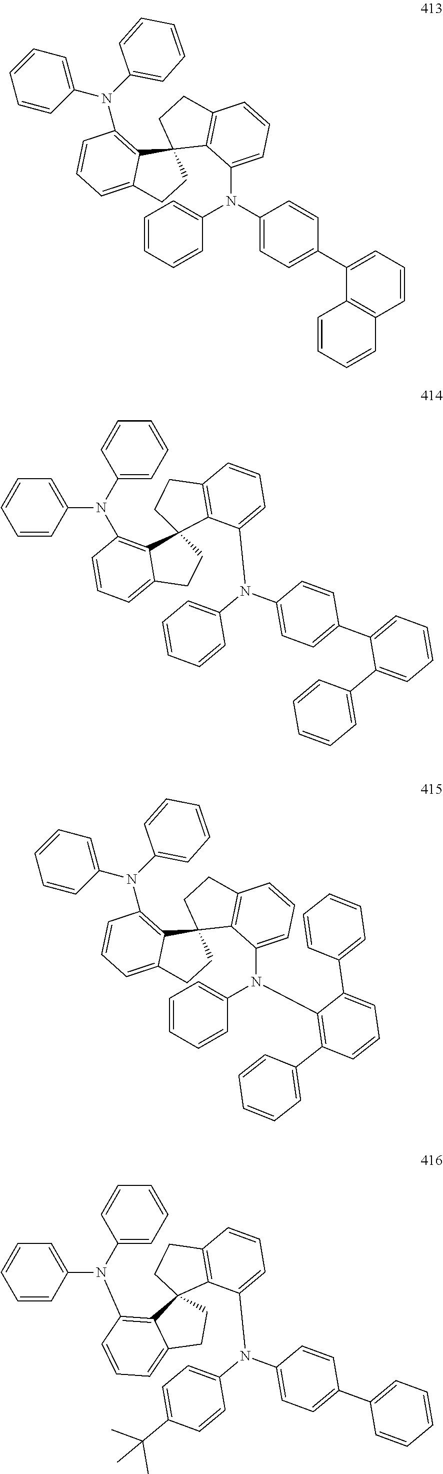 [SMILES: c1ccc(N(c2ccccc2)c2cccc3c2[C@@]2(CC3)CCc3cccc(N(c4ccccc4)c4ccc(-c5cccc6ccccc56)cc4)c32)cc1 ; c1ccc(-c2ccccc2-c2ccc(N(c3ccccc3)c3cccc4c3[C@]3(CCc5cccc(N(c6ccccc6)c6ccccc6)c53)CC4)cc2)cc1 ; c1ccc(-c2cccc(-c3ccccc3)c2N(c2ccccc2)c2cccc3c2[C@]2(CCc4cccc(N(c5ccccc5)c5ccccc5)c42)CC3)cc1 ; CC(C)(C)c1ccc(N(c2ccc(-c3ccccc3)cc2)c2cccc3c2[C@]2(CCc4cccc(N(c5ccccc5)c5ccccc5)c42)CC3)cc1]

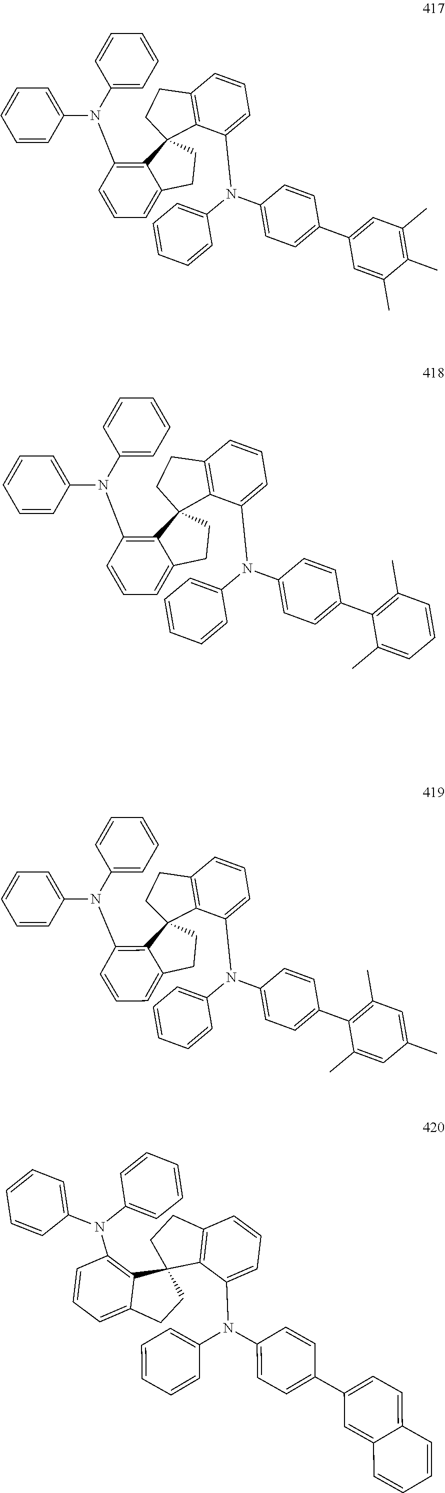 [SMILES: Cc1cc(-c2ccc(N(c3ccccc3)c3cccc4c3[C@]3(CCc5cccc(N(c6ccccc6)c6ccccc6)c53)CC4)cc2)cc(C)c1C ; Cc1cccc(C)c1-c1ccc(N(c2ccccc2)c2cccc3c2[C@]2(CCc4cccc(N(c5ccccc5)c5ccccc5)c42)CC3)cc1 ; Cc1cc(C)c(-c2ccc(N(c3ccccc3)c3cccc4c3[C@]3(CCc5cccc(N(c6ccccc6)c6ccccc6)c53)CC4)cc2)c(C)c1 ; c1ccc(N(c2ccccc2)c2cccc3c2[C@@]2(CC3)CCc3cccc(N(c4ccccc4)c4ccc(-c5ccc6ccccc6c5)cc4)c32)cc1]

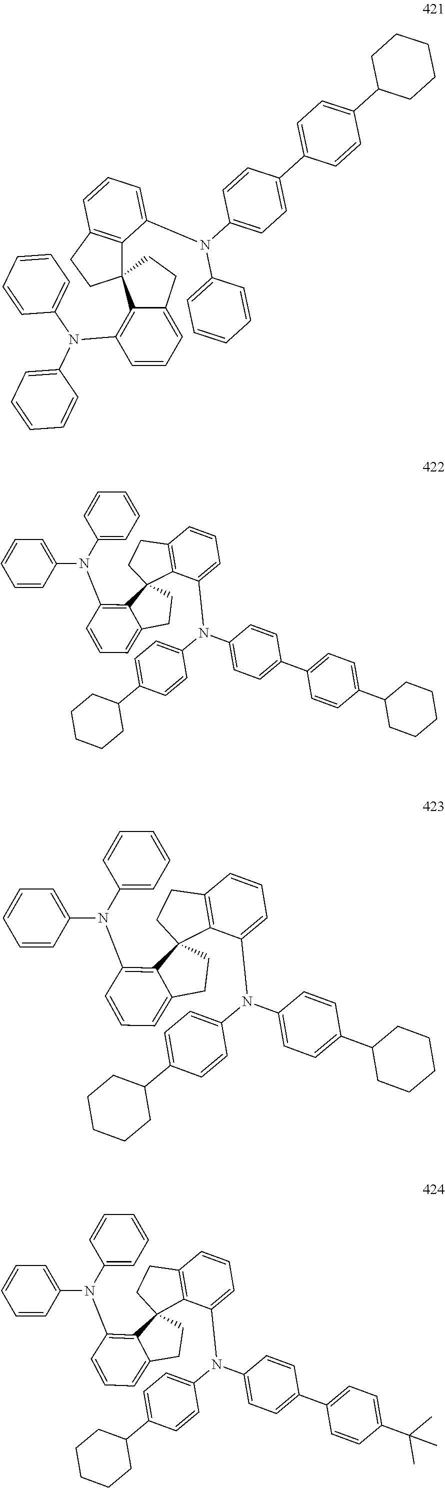 [SMILES: c1ccc(N(c2ccccc2)c2cccc3c2[C@@]2(CC3)CCc3cccc(N(c4ccccc4)c4ccc(-c5ccc(C6CCCCC6)cc5)cc4)c32)cc1 ; c1ccc(N(c2ccccc2)c2cccc3c2[C@@]2(CC3)CCc3cccc(N(c4ccc(-c5ccc(C6CCCCC6)cc5)cc4)c4ccc(C5CCCCC5)cc4)c32)cc1 ; c1ccc(N(c2ccccc2)c2cccc3c2[C@@]2(CC3)CCc3cccc(N(c4ccc(C5CCCCC5)cc4)c4ccc(C5CCCCC5)cc4)c32)cc1 ; CC(C)(C)c1ccc(-c2ccc(N(c3ccc(C4CCCCC4)cc3)c3cccc4c3[C@]3(CCc5cccc(N(c6ccccc6)c6ccccc6)c53)CC4)cc2)cc1]

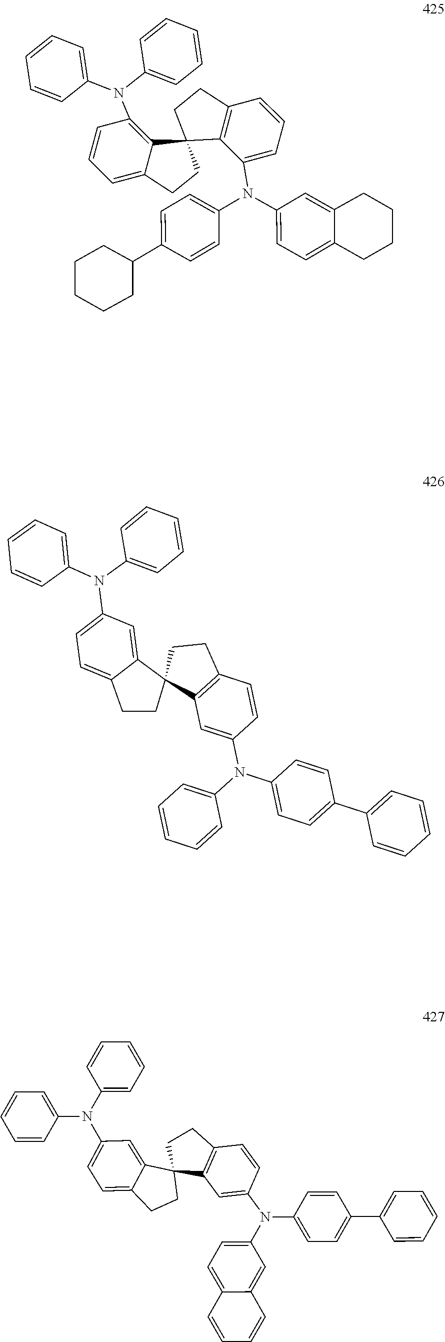 [SMILES: c1ccc(N(c2ccccc2)c2cccc3c2[C@@]2(CC3)CCc3cccc(N(c4ccc(C5CCCCC5)cc4)c4ccc5c(c4)CCCC5)c32)cc1 ; c1ccc(-c2ccc(N(c3ccccc3)c3ccc4c(c3)[C@]3(CCc5ccc(N(c6ccccc6)c6ccccc6)cc53)CC4)cc2)cc1 ; c1ccc(-c2ccc(N(c3ccc4c(c3)[C@]3(CCc5ccc(N(c6ccccc6)c6ccccc6)cc53)CC4)c3ccc4ccccc4c3)cc2)cc1]

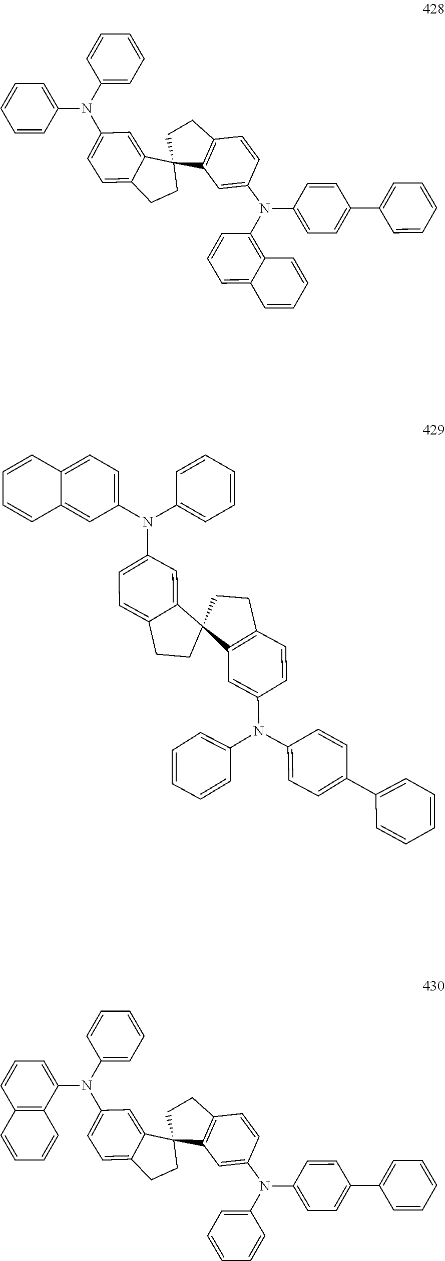 [SMILES: c1ccc(-c2ccc(N(c3ccc4c(c3)[C@]3(CCc5ccc(N(c6ccccc6)c6ccccc6)cc53)CC4)c3cccc4ccccc34)cc2)cc1 ; c1ccc(-c2ccc(N(c3ccccc3)c3ccc4c(c3)[C@@]3(CC4)CCc4ccc(N(c5ccccc5)c5ccc6ccccc6c5)cc43)cc2)cc1 ; c1ccc(-c2ccc(N(c3ccccc3)c3ccc4c(c3)[C@@]3(CC4)CCc4ccc(N(c5ccccc5)c5cccc6ccccc56)cc43)cc2)cc1]

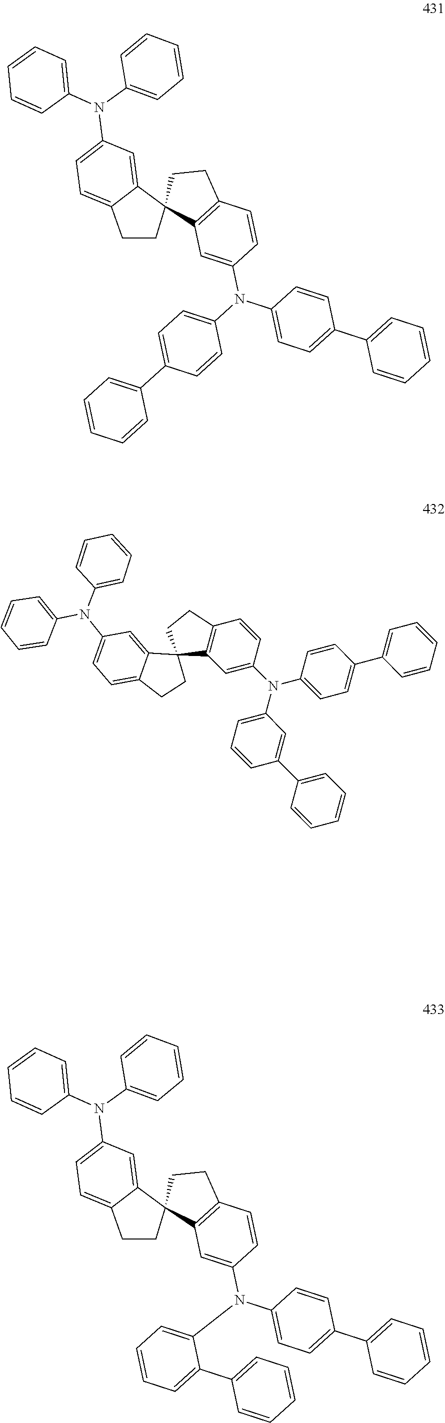 [SMILES: c1ccc(-c2ccc(N(c3ccc(-c4ccccc4)cc3)c3ccc4c(c3)[C@]3(CCc5ccc(N(c6ccccc6)c6ccccc6)cc53)CC4)cc2)cc1 ; c1ccc(-c2ccc(N(c3cccc(-c4ccccc4)c3)c3ccc4c(c3)[C@]3(CCc5ccc(N(c6ccccc6)c6ccccc6)cc53)CC4)cc2)cc1 ; c1ccc(-c2ccc(N(c3ccc4c(c3)[C@]3(CCc5ccc(N(c6ccccc6)c6ccccc6)cc53)CC4)c3ccccc3-c3ccccc3)cc2)cc1]

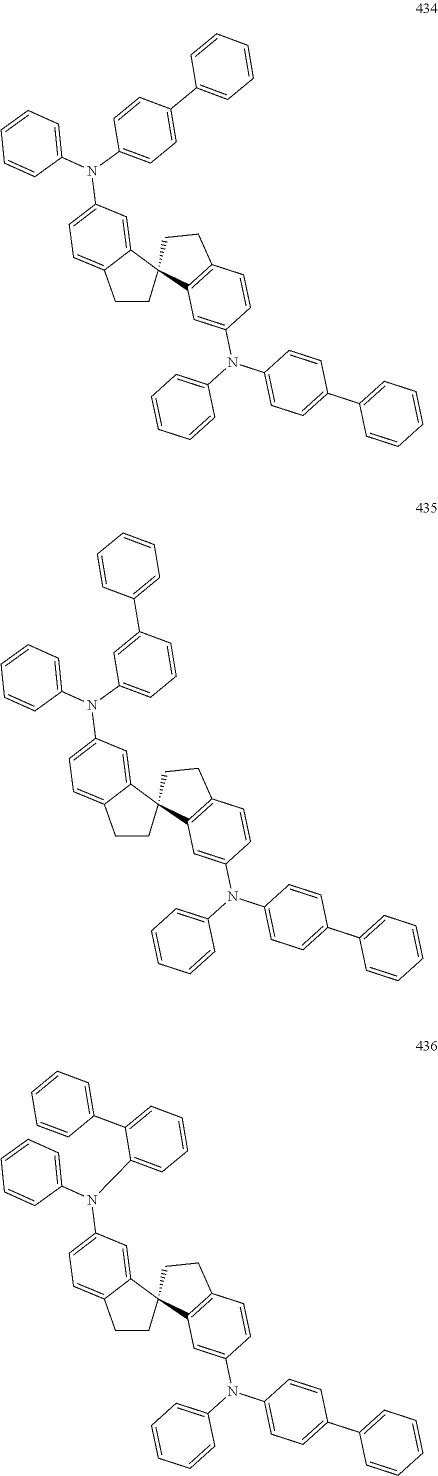

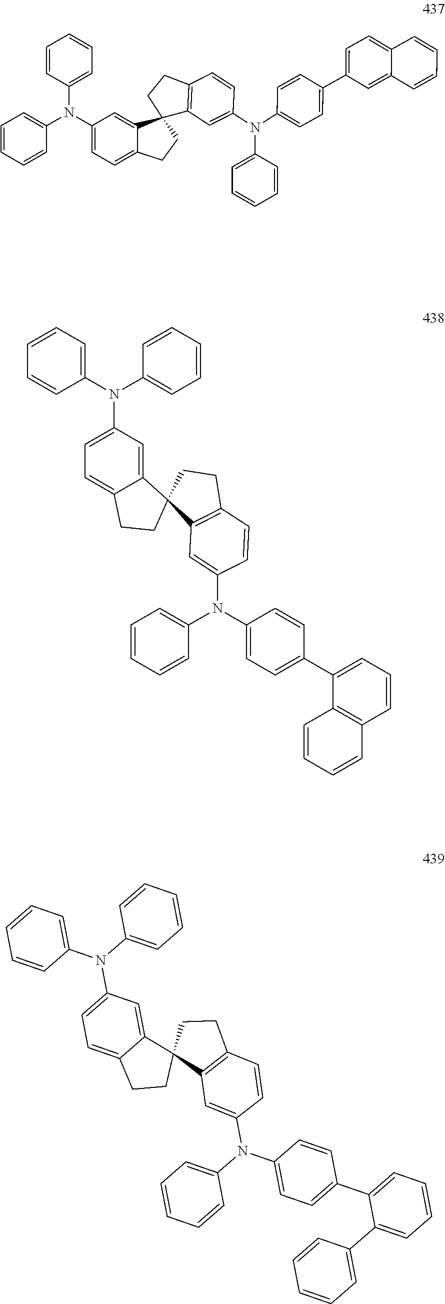 [SMILES: c1ccc(N(c2ccccc2)c2ccc3c(c2)[C@@]2(CC3)CCc3ccc(N(c4ccccc4)c4ccc(-c5ccc6ccccc6c5)cc4)cc32)cc1 ; c1ccc(N(c2ccccc2)c2ccc3c(c2)[C@@]2(CC3)CCc3ccc(N(c4ccccc4)c4ccc(-c5cccc6ccccc56)cc4)cc32)cc1 ; c1ccc(-c2ccccc2-c2ccc(N(c3ccccc3)c3ccc4c(c3)[C@]3(CCc5ccc(N(c6ccccc6)c6ccccc6)cc53)CC4)cc2)cc1]

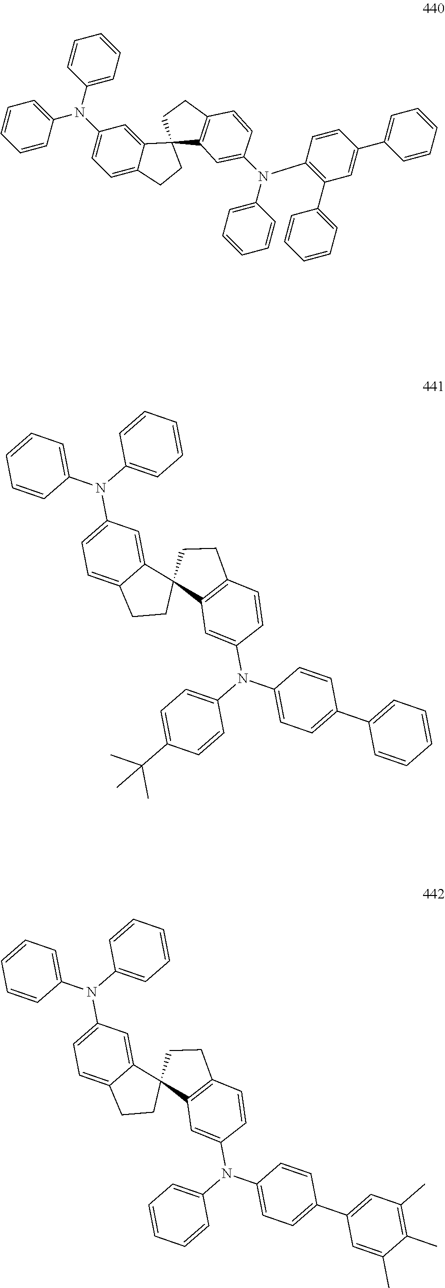 [SMILES: c1ccc(-c2ccc(N(c3ccccc3)c3ccc4c(c3)[C@]3(CCc5ccc(N(c6ccccc6)c6ccccc6)cc53)CC4)c(-c3ccccc3)c2)cc1 ; CC(C)(C)c1ccc(N(c2ccc(-c3ccccc3)cc2)c2ccc3c(c2)[C@]2(CCc4ccc(N(c5ccccc5)c5ccccc5)cc42)CC3)cc1 ; Cc1cc(-c2ccc(N(c3ccccc3)c3ccc4c(c3)[C@]3(CCc5ccc(N(c6ccccc6)c6ccccc6)cc53)CC4)cc2)cc(C)c1C]

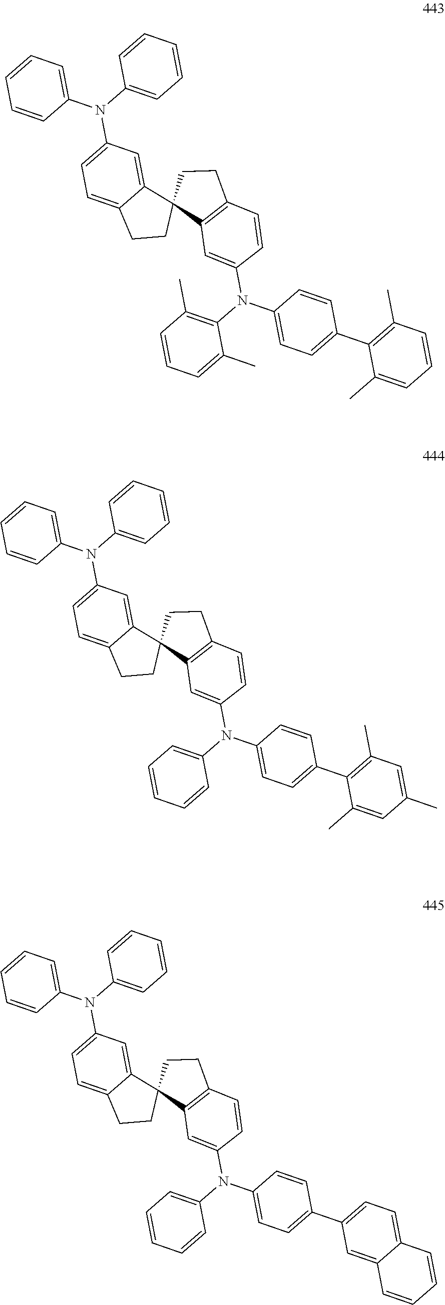 [SMILES: Cc1cccc(C)c1-c1ccc(N(c2ccc3c(c2)[C@]2(CCc4ccc(N(c5ccccc5)c5ccccc5)cc42)CC3)c2c(C)cccc2C)cc1 ; Cc1cc(C)c(-c2ccc(N(c3ccccc3)c3ccc4c(c3)[C@]3(CCc5ccc(N(c6ccccc6)c6ccccc6)cc53)CC4)cc2)c(C)c1 ; c1ccc(N(c2ccccc2)c2ccc3c(c2)[C@@]2(CC3)CCc3ccc(N(c4ccccc4)c4ccc(-c5ccc6ccccc6c5)cc4)cc32)cc1]

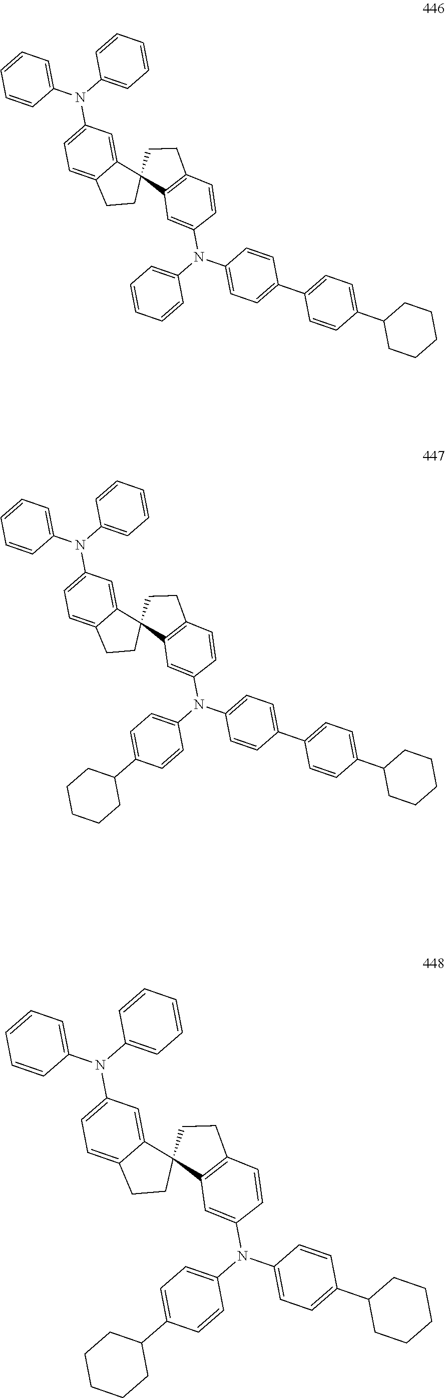 [SMILES: c1ccc(N(c2ccccc2)c2ccc3c(c2)[C@@]2(CC3)CCc3ccc(N(c4ccccc4)c4ccc(-c5ccc(C6CCCCC6)cc5)cc4)cc32)cc1 ; c1ccc(N(c2ccccc2)c2ccc3c(c2)[C@@]2(CC3)CCc3ccc(N(c4ccc(-c5ccc(C6CCCCC6)cc5)cc4)c4ccc(C5CCCCC5)cc4)cc32)cc1 ; c1ccc(N(c2ccccc2)c2ccc3c(c2)[C@@]2(CC3)CCc3ccc(N(c4ccc(C5CCCCC5)cc4)c4ccc(C5CCCCC5)cc4)cc32)cc1]

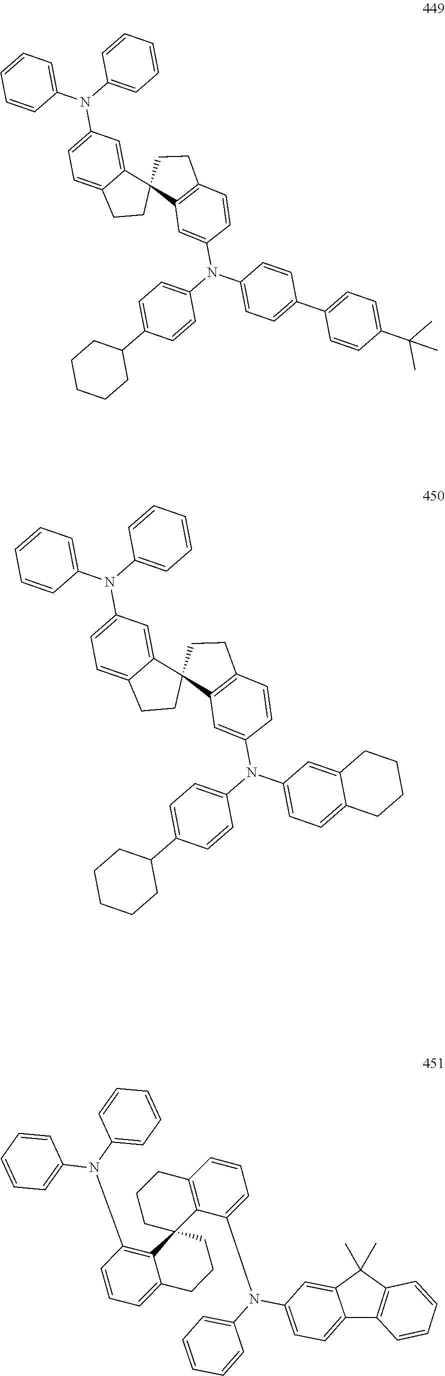 [SMILES: CC(C)(C)c1ccc(-c2ccc(N(c3ccc(C4CCCCC4)cc3)c3ccc4c(c3)[C@]3(CCc5ccc(N(c6ccccc6)c6ccccc6)cc53)CC4)cc2)cc1 ; c1ccc(N(c2ccccc2)c2ccc3c(c2)[C@@]2(CC3)CCc3ccc(N(c4ccc(C5CCCCC5)cc4)c4ccc5c(c4)CCCC5)cc32)cc1 ; CC1(C)c2ccccc2-c2ccc(N(c3ccccc3)c3cccc4c3[C@]3(CCCc5cccc(N(c6ccccc6)c6ccccc6)c53)CCC4)cc21]

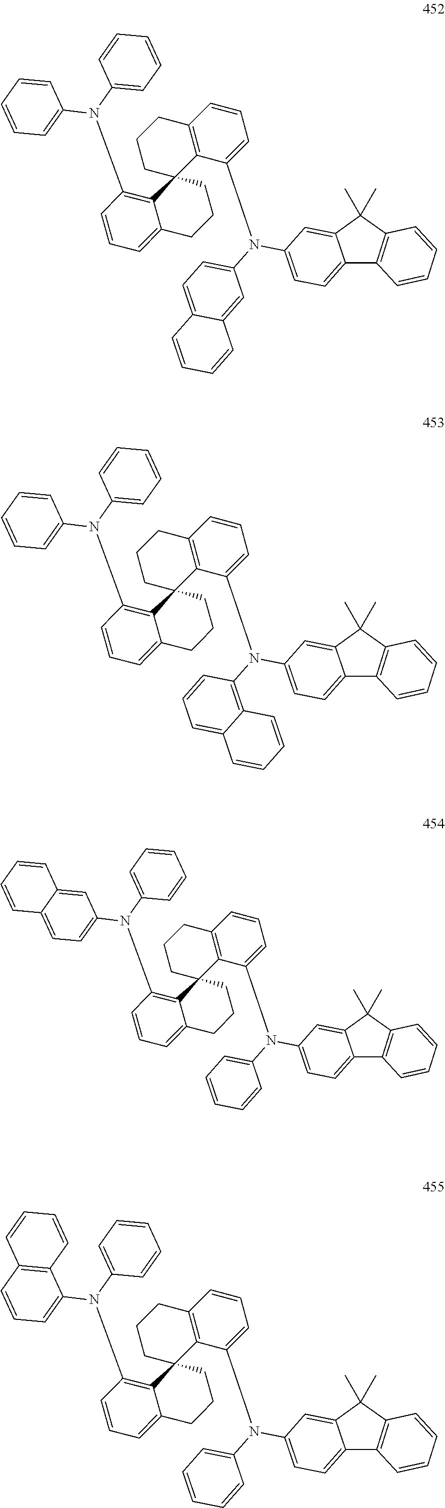 [SMILES: CC1(C)c2ccccc2-c2ccc(N(c3ccc4ccccc4c3)c3cccc4c3[C@]3(CCCc5cccc(N(c6ccccc6)c6ccccc6)c53)CCC4)cc21 ; CC1(C)c2ccccc2-c2ccc(N(c3cccc4c3[C@]3(CCCc5cccc(N(c6ccccc6)c6ccccc6)c53)CCC4)c3cccc4ccccc34)cc21 ; CC1(C)c2ccccc2-c2ccc(N(c3ccccc3)c3cccc4c3[C@@]3(CCC4)CCCc4cccc(N(c5ccccc5)c5ccc6ccccc6c5)c43)cc21 ; CC1(C)c2ccccc2-c2ccc(N(c3ccccc3)c3cccc4c3[C@@]3(CCC4)CCCc4cccc(N(c5ccccc5)c5cccc6ccccc56)c43)cc21]

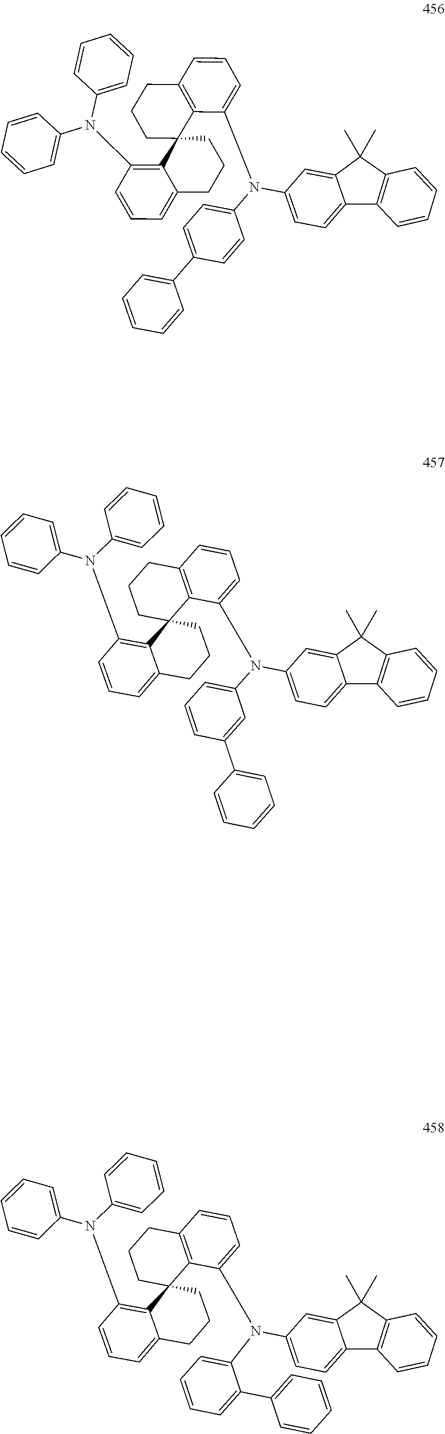 [SMILES: CC1(C)c2ccccc2-c2ccc(N(c3ccc(-c4ccccc4)cc3)c3cccc4c3[C@]3(CCCc5cccc(N(c6ccccc6)c6ccccc6)c53)CCC4)cc21 ; CC1(C)c2ccccc2-c2ccc(N(c3cccc(-c4ccccc4)c3)c3cccc4c3[C@]3(CCCc5cccc(N(c6ccccc6)c6ccccc6)c53)CCC4)cc21 ; CC1(C)c2ccccc2-c2ccc(N(c3ccccc3-c3ccccc3)c3cccc4c3[C@]3(CCCc5cccc(N(c6ccccc6)c6ccccc6)c53)CCC4)cc21]

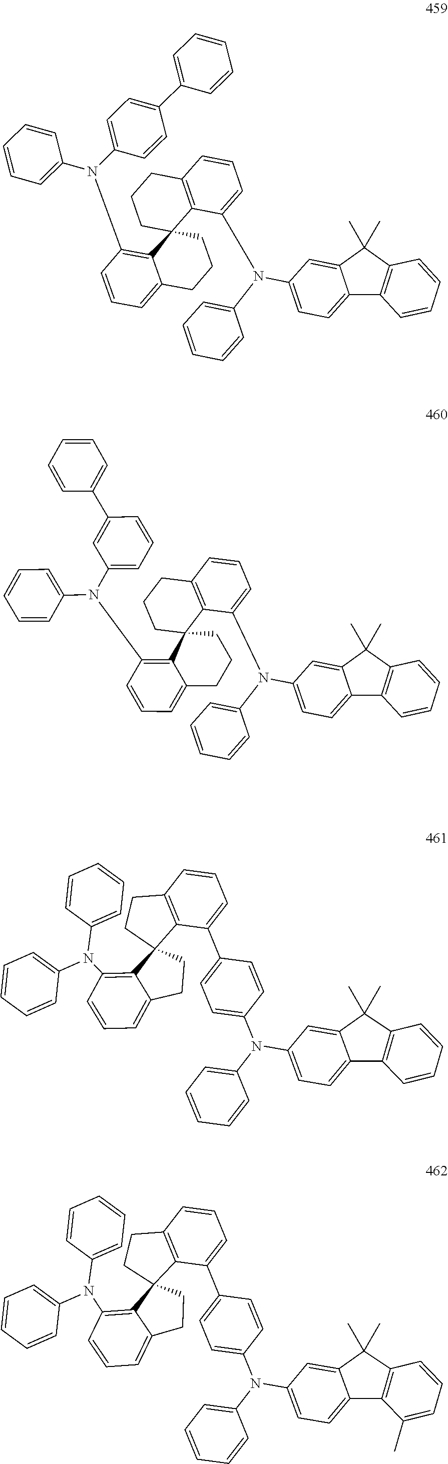 [SMILES: CC1(C)c2ccccc2-c2ccc(N(c3ccccc3)c3cccc4c3[C@]3(CCCc5cccc(N(c6ccccc6)c6ccc(-c7ccccc7)cc6)c53)CCC4)cc21 ; CC1(C)c2ccccc2-c2ccc(N(c3ccccc3)c3cccc4c3[C@]3(CCCc5cccc(N(c6ccccc6)c6cccc(-c7ccccc7)c6)c53)CCC4)cc21 ; CC1(C)c2ccccc2-c2ccc(N(c3ccccc3)c3ccc(-c4cccc5c4[C@@]4(CC5)CCc5cccc(N(c6ccccc6)c6ccccc6)c54)cc3)cc21 ; Cc1cccc2c1-c1ccc(N(c3ccccc3)c3ccc(-c4cccc5c4[C@@]4(CC5)CCc5cccc(N(c6ccccc6)c6ccccc6)c54)cc3)cc1C2(C)C]

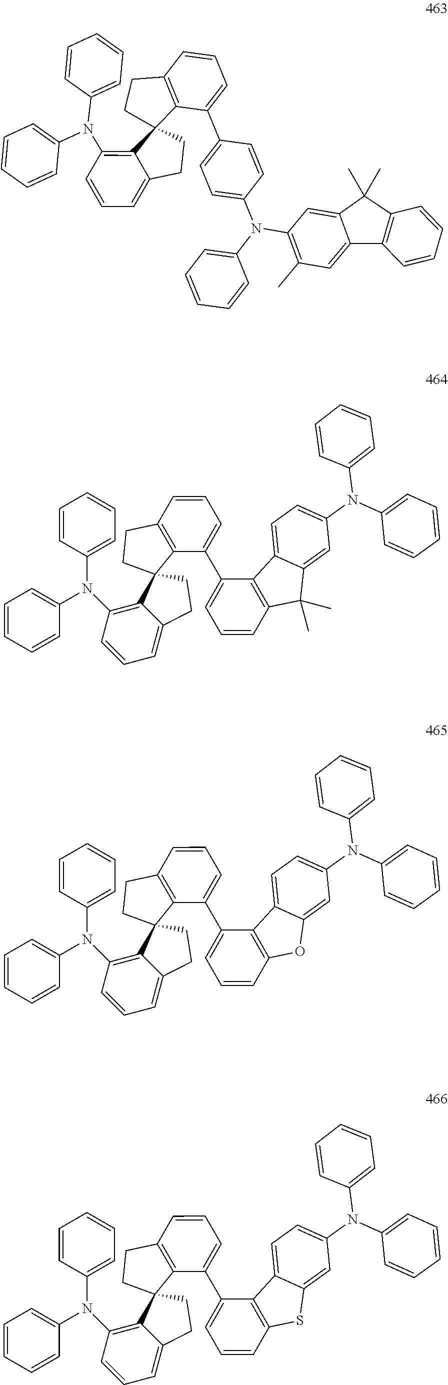 [SMILES: Cc1cc2c(cc1N(c1ccccc1)c1ccc(-c3cccc4c3[C@@]3(CC4)CCc4cccc(N(c5ccccc5)c5ccccc5)c43)cc1)C(C)(C)c1ccccc1-2 ; CC1(C)c2cc(N(c3ccccc3)c3ccccc3)ccc2-c2c(-c3cccc4c3[C@@]3(CC4)CCc4cccc(N(c5ccccc5)c5ccccc5)c43)cccc21 ; c1ccc(N(c2ccccc2)c2ccc3c(c2)oc2cccc(-c4cccc5c4[C@@]4(CC5)CCc5cccc(N(c6ccccc6)c6ccccc6)c54)c23)cc1 ; c1ccc(N(c2ccccc2)c2ccc3c(c2)sc2cccc(-c4cccc5c4[C@@]4(CC5)CCc5cccc(N(c6ccccc6)c6ccccc6)c54)c23)cc1]

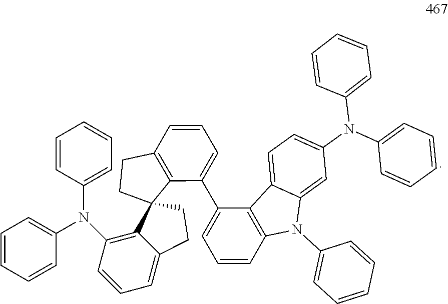 [SMILES: c1ccc(N(c2ccccc2)c2ccc3c4c(-c5cccc6c5[C@@]5(CC6)CCc6cccc(N(c7ccccc7)c7ccccc7)c65)cccc4n(-c4ccccc4)c3c2)cc1]

The amine compound of one or more embodiments may include a spiro-bonded linker of two hydrocarbon rings, and may reduce intermolecular interaction and have low packing density. Accordingly, the amine compound of one or more embodiments, represented by Formula 1 may have low refractive index properties. In some embodiments, in the amine compound of one or more embodiments, the glass transition temperature of the amine compound may be markedly improved, and the energy level of the highest occupied molecular orbital (HOMO) of a molecule may be diversely (e.g., variously) changed, by diversely (e.g., variously) changing the substitution positions of the first amine group and the second amine group connected at the linker. Accordingly, the light emitting element ED of one or more embodiments may diversely change the hole injection barrier between a first electrode EL1 and a hole transport region HTR and may provide a suitable energy level between the hole transport region HTR and an emission layer EML, thereby controlling to improve (e.g., facilitating the improvement of) exciton production efficiency in the emission layer EML. Accordingly, if the amine compound according to one or more embodiments is applied in the hole transport region HTR of the light emitting element ED, a light emitting element with high efficiency, a low driving voltage, high luminance, and long lifetime may be achieved.

In one or more embodiments, the hole transport region HTR may further include a compound represented by Formula H-1 below. For example, the light emitting element ED of one or more embodiments may further include a compound represented by Formula H-1 in the hole transport layer of the hole transport region HTR. Referring to FIG. 5, the light emitting element ED of one or more embodiments may include the amine compound of one or more embodiments in a first hole transport layer HTL-1 and a third hole transport layer HTL-3, and may include the compound represented by Formula H-1 in a second hole transport layer HTL-2.

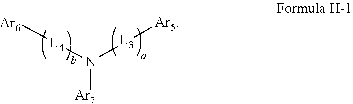

In Formula H-1 above, L₃ and L₄ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10. If “a” and/or “b” is (e.g., each) an integer of 2 or more, multiple L₃ and L₄ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Ar₅ and Ar₆ may be each independently a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar₇ may be a substituted or unsubstituted aryl group of 6 to 30 ring-forming carbon atoms.

The compound represented by Formula H-1 may be a monoamine compound. In one or more embodiments, the compound represented by Formula H-1 may be a diamine compound in which at least one selected from Ar₅ to Ar₇ includes an amine group as a substituent. In some embodiments, the compound represented by Formula H-1 may be a carbazole-based compound in which at least one selected from Ar₅ and Ar₆ includes a substituted or unsubstituted carbazole group, or a fluorene-based compound in which at least one selected from Ar₅ and Ar₆ includes a substituted or unsubstituted fluorene group.

The compound represented by Formula H-1 may be represented by any one selected from compounds in Compound Group H below. However, the compounds listed in Compound Group H are only illustrations, and the compound represented by Formula H-1 is not limited to the compounds represented in Compound Group H below.

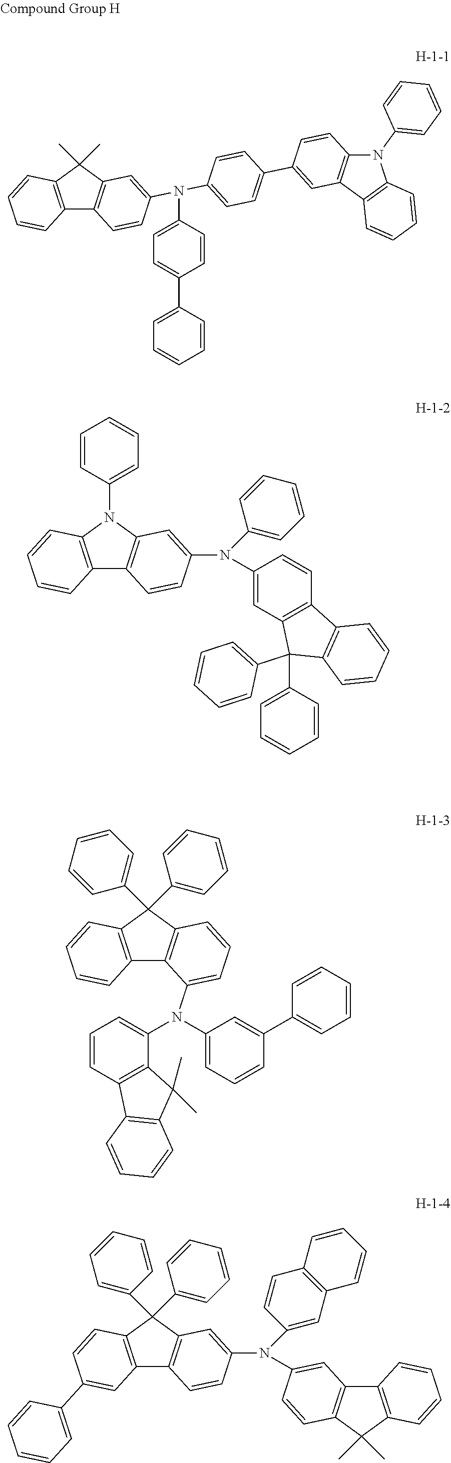 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

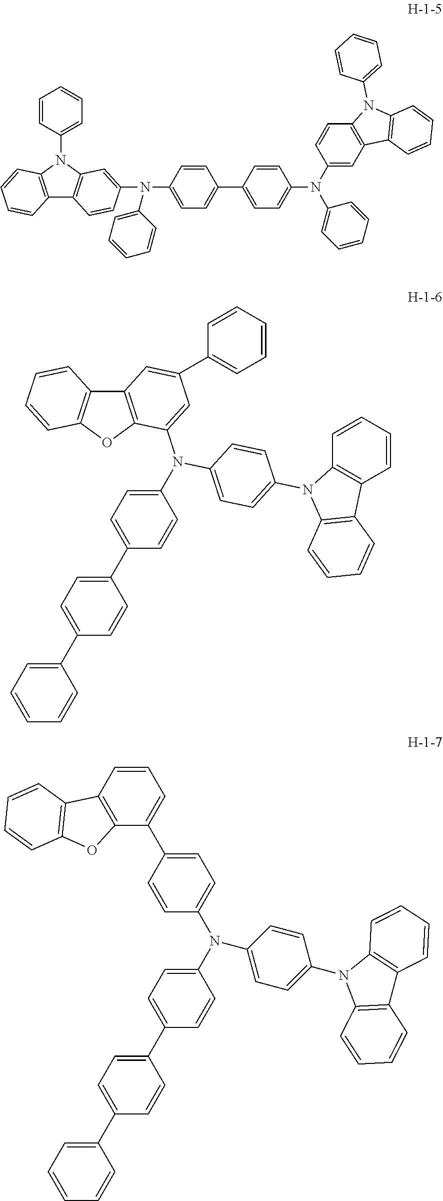 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

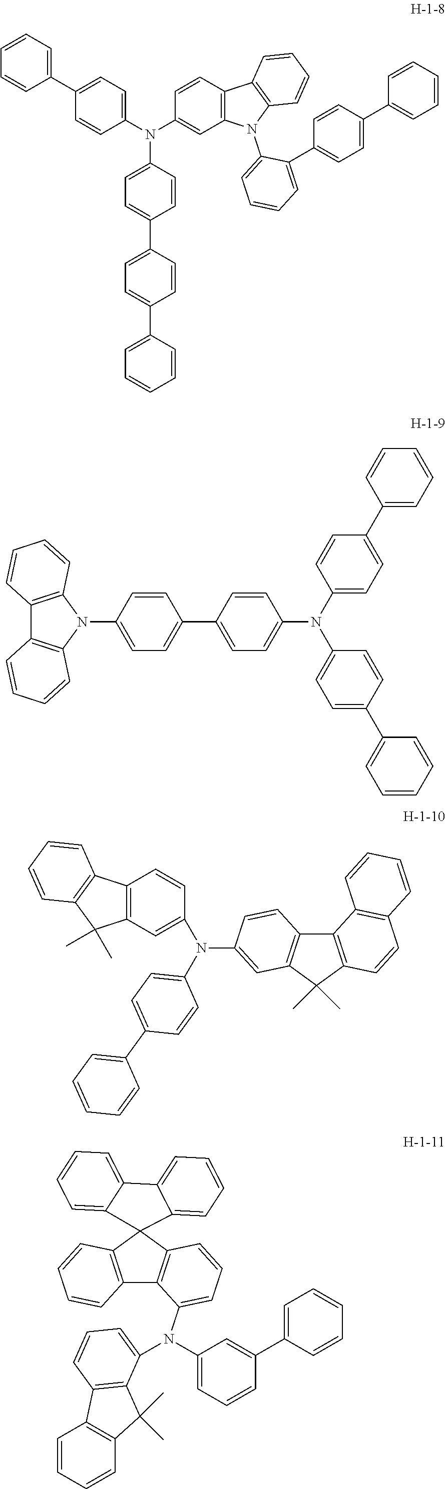

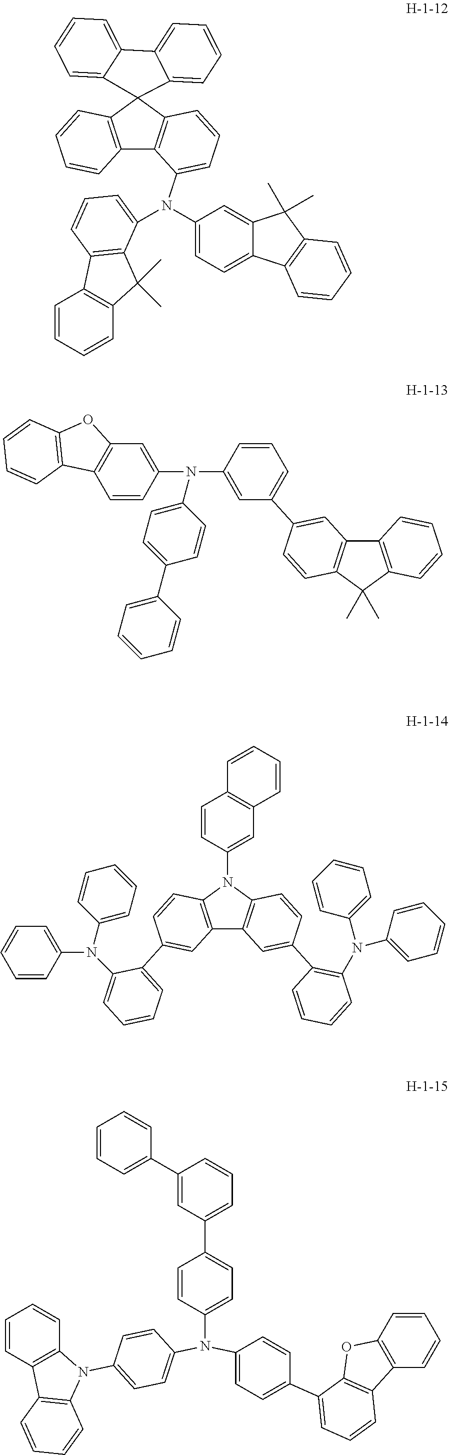 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

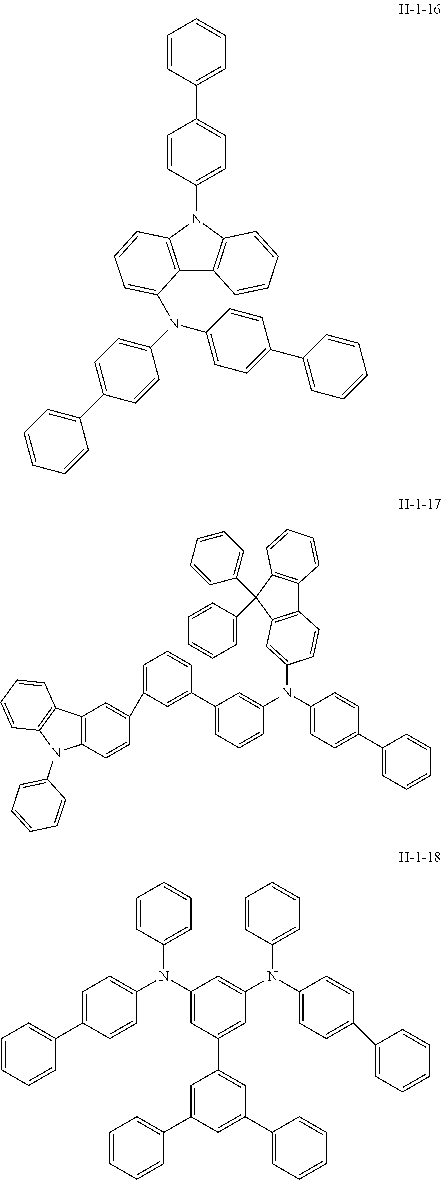 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

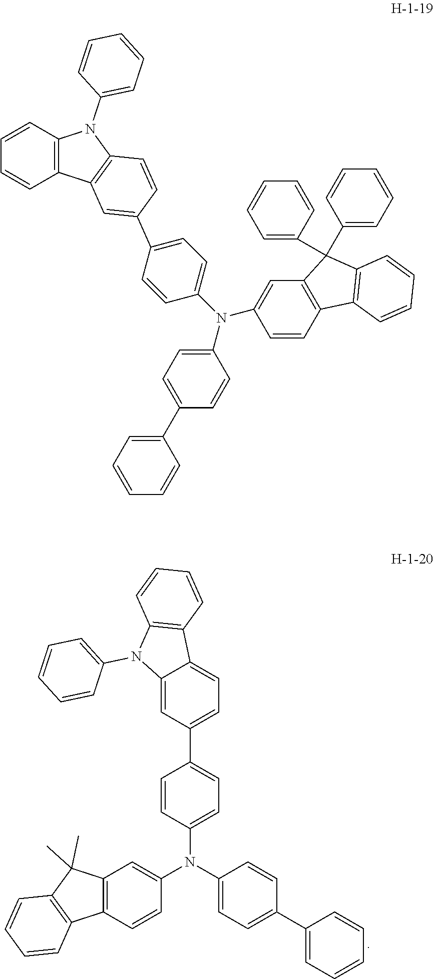 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; CC1(C)c2ccccc2-c2ccc(N(c3ccc(-c4ccccc4)cc3)c3ccc(-c4ccc5c6ccccc6n(-c6ccccc6)c5c4)cc3)cc21]

The hole transport region HTR may further include any of the compounds explained below.

The hole transport region HTR may include a phthalocyanine compound (such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD)), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB of NPD), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HATCN).

The hole transport region HTR may include carbazole derivatives (such as N-phenyl carbazole and/or polyvinyl carbazole), fluorene-based derivatives, N,N′-bis(3-methylphenyl)-N,N′-diphenyl-[1,1′-biphenyl]-4,4′-diamine (TPD), triphenylamine-based derivatives (such as 4,4′,4″-tris(N-carbazolyl)triphenylamine (TCTA), N,N′-di(1-naphthalene-1-yl)-N,N′-diphenyl-benzidine (NPB)), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), and/or 1,3-bis(1,8-dimethyl-9H-carbazol-9-yl)benzene (mDCP).

The hole transport region HTR may include the compounds of the hole transport region HTR in at least one selected from the hole injection layer HIL, hole transport layer HTL, and electron blocking layer EBL.

The thickness of the hole transport region HTR may be from about 100 Å to about 10,000 Å, for example, from about 100 Å to about 5,000 Å. In case where the hole transport region HTR includes a hole injection layer HIL, the thickness of the hole injection layer HIL may be, for example, from about 30 Å to about 1,000 Å. In case where the hole transport region HTR includes a hole transport layer HTL, the thickness of the hole transport layer HTL may be from about 30 Å to about 1,000 Å. For example, in case where the hole transport region HTR includes an electron blocking layer EBL, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy any of the above-described respective ranges, satisfactory or suitable hole transport properties may be achieved without substantial increase of a driving voltage.

The hole transport region HTR may further include a charge generating material to increase conductivity, in addition to the above-described materials. The charge generating material may be dispersed substantially uniformly or substantially non-uniformly in the hole transport region HTR. The charge generating material may be, for example, a p-dopant. The p-dopant may include at least one selected from metal halide compounds, quinone derivatives, metal oxides, and cyano group-containing compounds, without limitation. For example, the p-dopant may include metal halide compounds such as CuI and/or RbI, quinone derivatives such as tetracyanoquinodimethane (TCNQ) and/or 2,3,5,6-tetrafluoro-7,7,8,8-tetracyanoquinodimethane (F4-TCNQ), metal oxides such as tungsten oxide and/or molybdenum oxide, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include at least one selected from a buffer layer and an electron blocking layer EBL in addition to the hole injection layer HIL and the hole transport layer HTL. The buffer layer may compensate resonance distance according to the wavelength of light emitted from an emission layer EML and may increase emission efficiency. As materials included in the buffer layer, materials which may be included in the hole transport region HTR may be used. The electron blocking layer EBL is a layer playing the role of preventing or reducing the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

In the light emitting element ED of one or more embodiments, the emission layer EML may emit any one selected from red light, green light, blue light, white light and cyan light. The light emitting element ED of one or more embodiments includes the amine compound of one or more embodiments in the hole transport region HTR, and may show high efficiency and long-life characteristics in the emission region emitting (e.g., configured to emit) the light.

In the light emitting element ED of one or more embodiments, the emission layer EML may include anthracene derivative(s), pyrene derivative(s), fluoranthene derivative(s), chrysene derivative(s), dihydrobenzanthracene derivative(s), and/or triphenylene derivative(s). For example, the emission layer EML may include anthracene derivatives and/or pyrene derivatives.

In the light emitting elements ED of embodiments, shown in FIG. 3 to FIG. 7, the emission layer EML may include a host and a dopant, and the emission layer EML may include a compound represented by Formula E-1 below. The compound represented by Formula E-1 below may be used as a fluorescence host material.

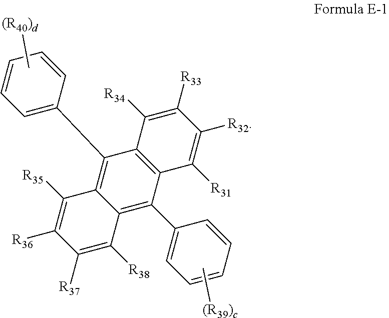

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. For example, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may be each independently an integer of 0 to 5.

Formula E-1 may be represented by any one selected from Compound E1 to Compound E19 below.

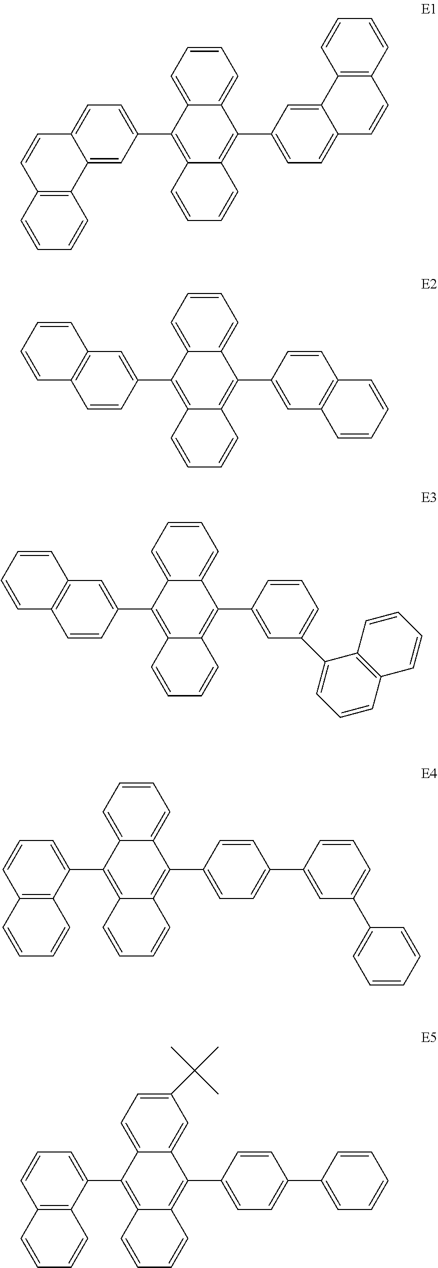

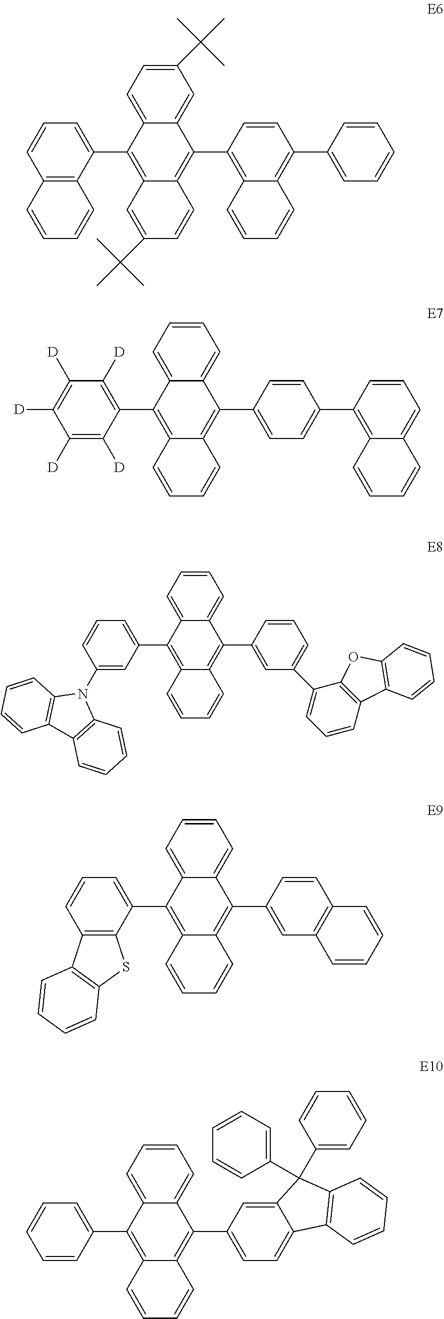

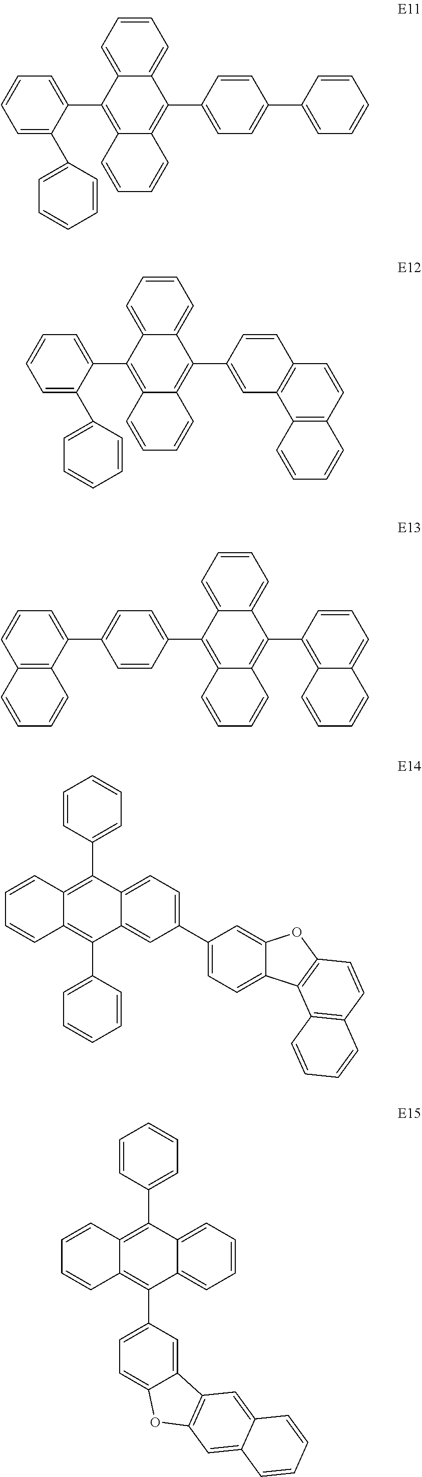

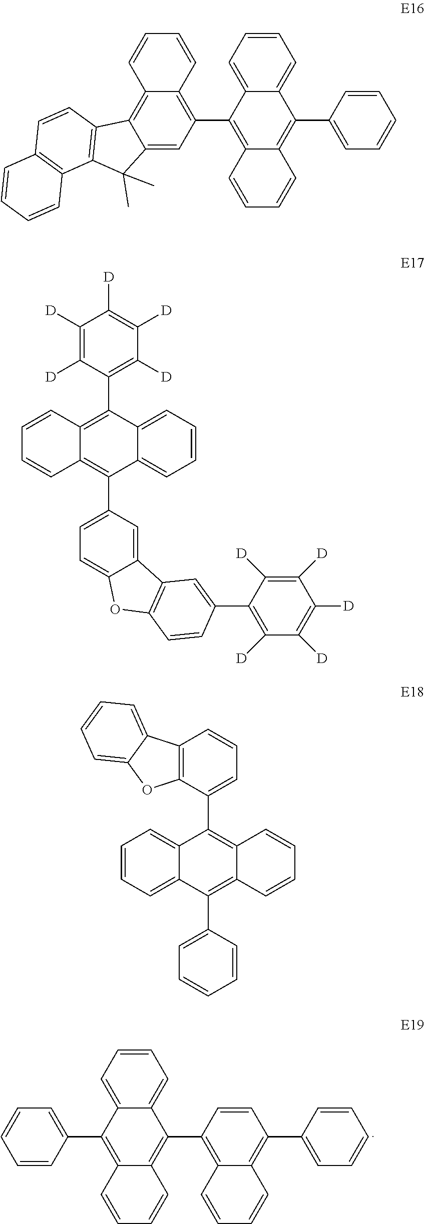

In the light emitting element ED of one or more embodiments, the emission layer EML may include a host and a dopant. For example, the emission layer EML may include one host and one dopant. In some embodiments, the emission layer EML may include two or more hosts, a sensitizer and a dopant. For example, the emission layer EML may include a hole transport host and an electron transport host. The emission layer EML may include a phosphorescence sensitizer and/or a thermally activated delayed fluorescence (TADF) sensitizer, as the sensitizer. If the emission layer EML includes a hole transport host, an electron transport host, a sensitizer and a dopant, the hole transport host and the electron transport host may form exciplexes, and energy transfer may occur from the exciplex to the sensitizer, and from the sensitizer to the dopant. However, this is an illustration, and the material included in the emission layer EML is not limited thereto.

In one or more embodiments, the emission layer EML may include at least one selected from a second compound represented by Formula HT-1, a third compound represented by Formula ET-1, and a fourth compound represented by Formula M-b below.

For example, the second compound may be used as the hole transport host material of the emission layer EML. The second compound represented by Formula HT-1 may include a carbazole moiety.

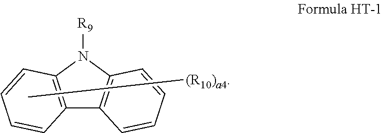

In Formula HT-1, a4 may be an integer of 0 to 8. If a4 is an integer of 2 or more, multiple R₁₀ may be the same, or at least one may be different. R₉ and R₁₀ may be each independently a hydrogen atom, a deuterium atom, a substituted or unsubstituted aryl group of 6 to 60 ring-forming carbon atoms, or a substituted or unsubstituted heteroaryl group of 2 to 60 ring-forming carbon atoms. For example, R₉ may be a substituted phenyl group, an unsubstituted dibenzofuran group, or a substituted fluorenyl group. R₁₀ may be a substituted or unsubstituted carbazole group.

The second compound may be represented by any one selected from compounds in Compound Group 2 below. In Compound Group 2, D is a deuterium atom, and Ph is a phenyl group.

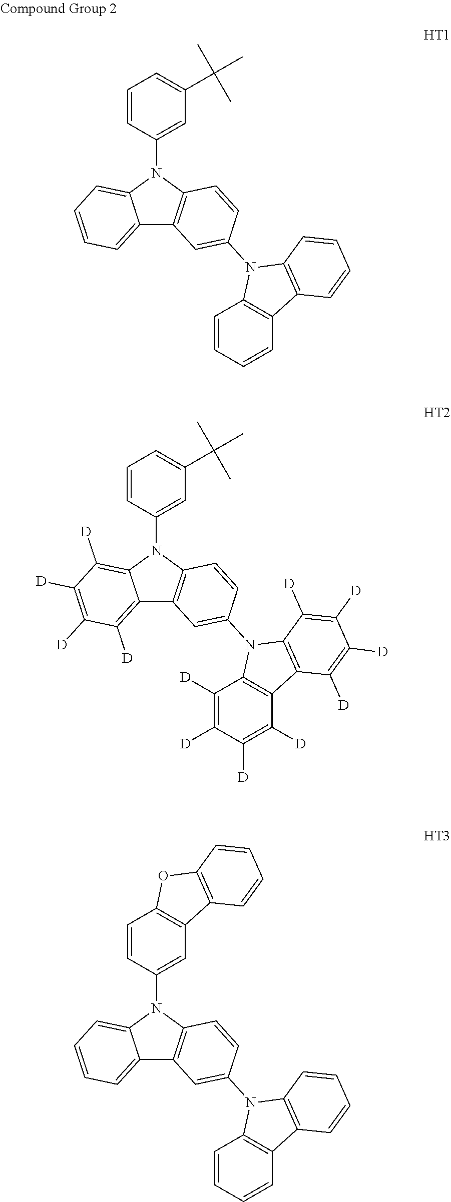 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

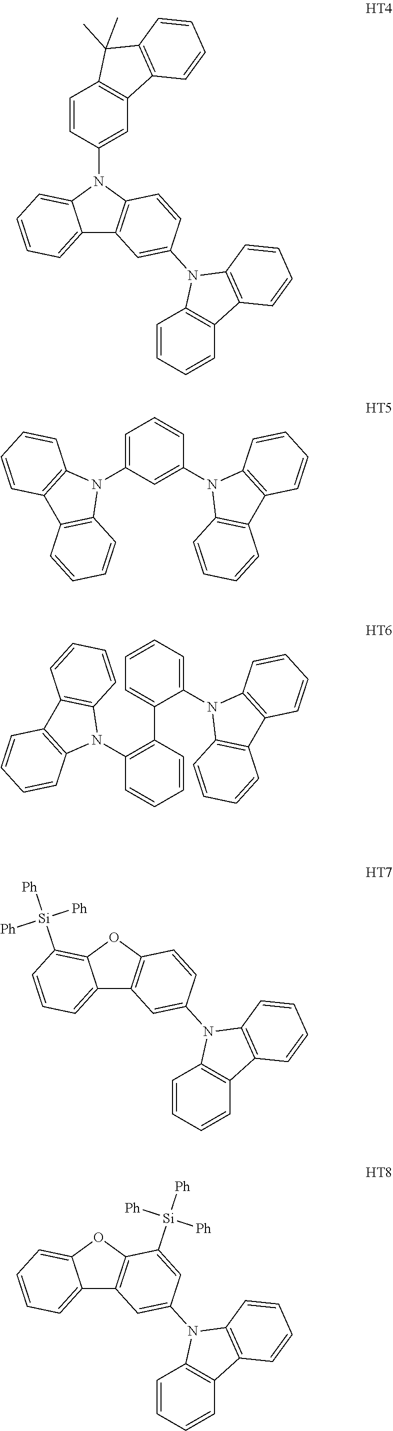 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

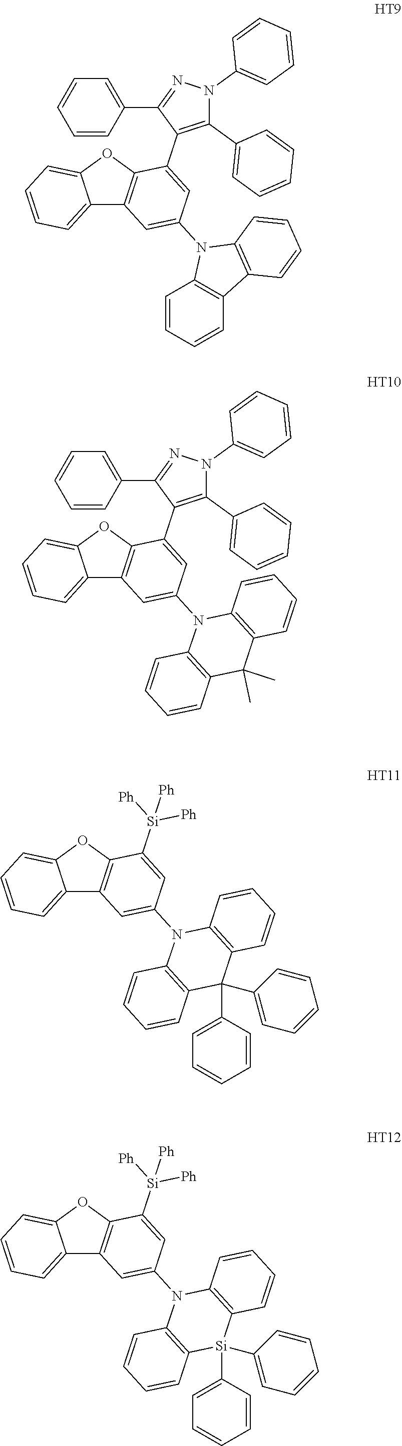 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

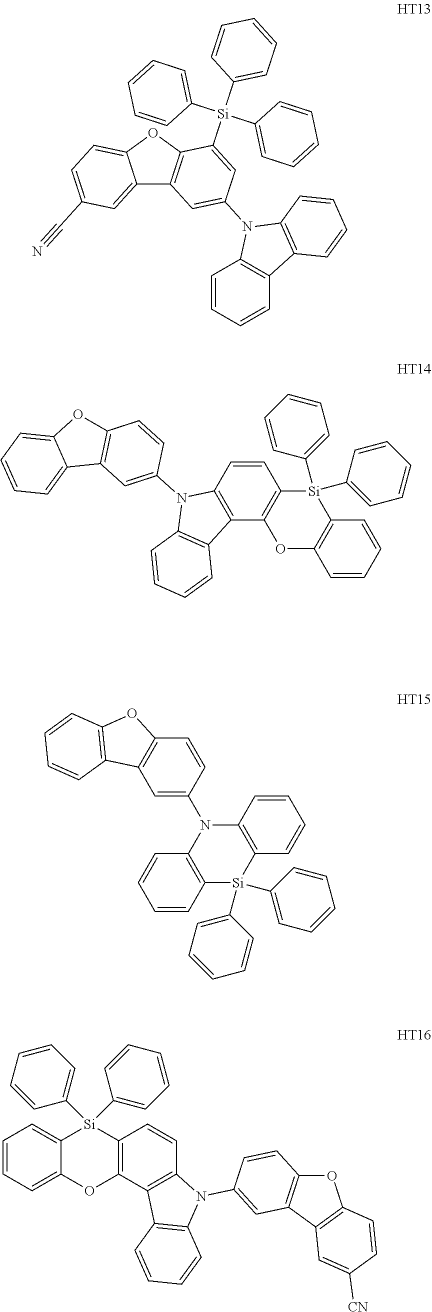 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

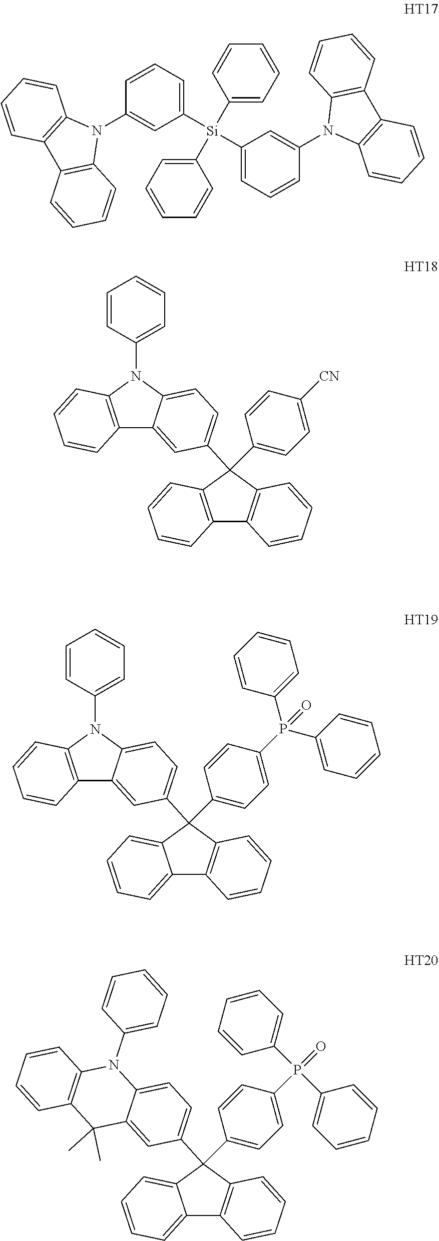 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

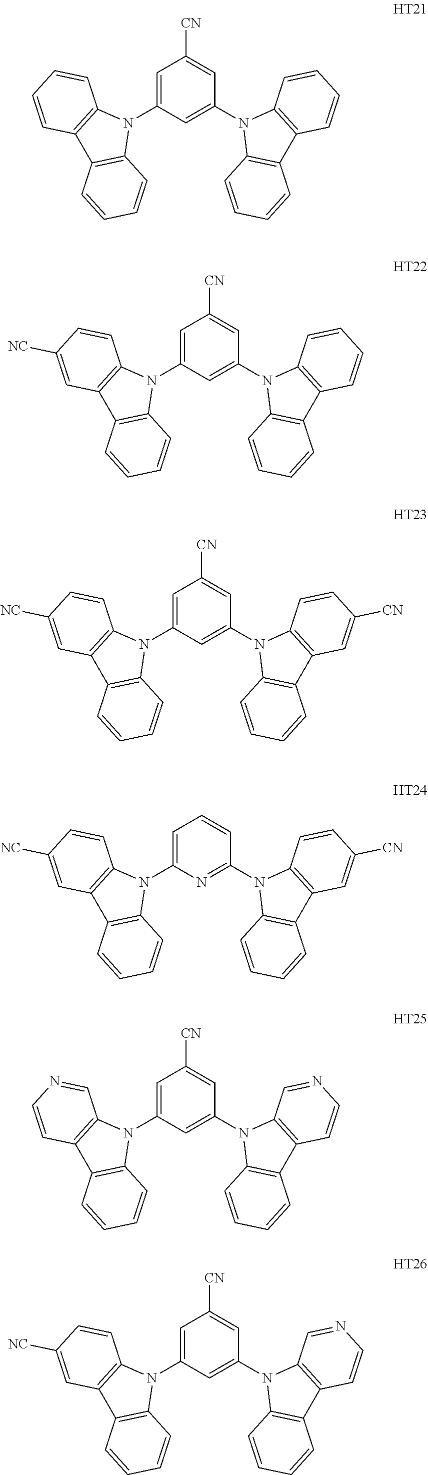 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

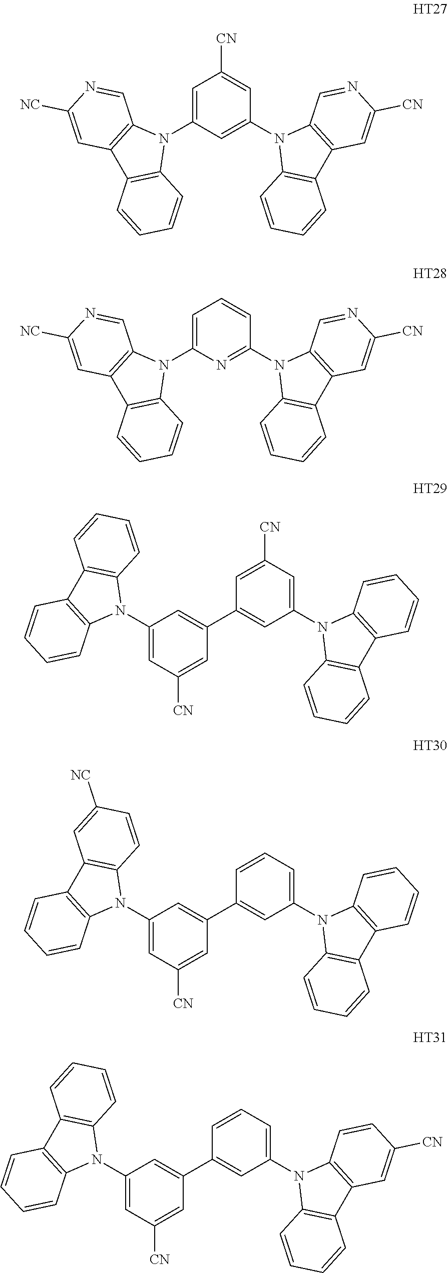 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

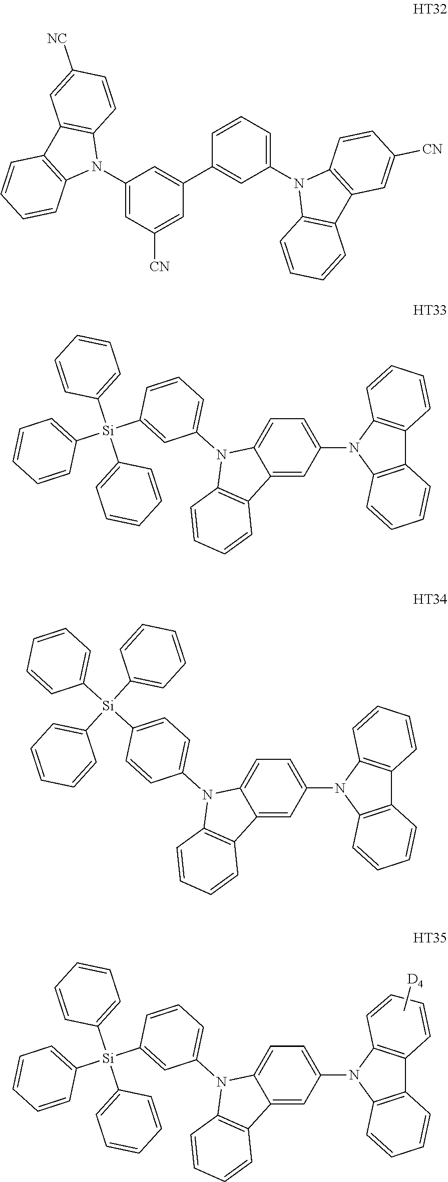

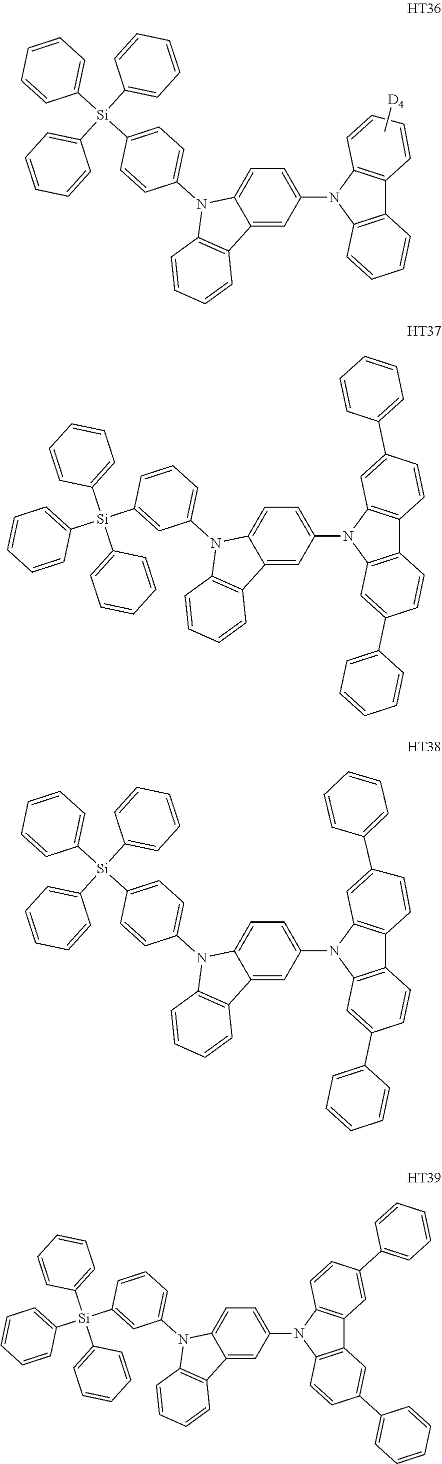

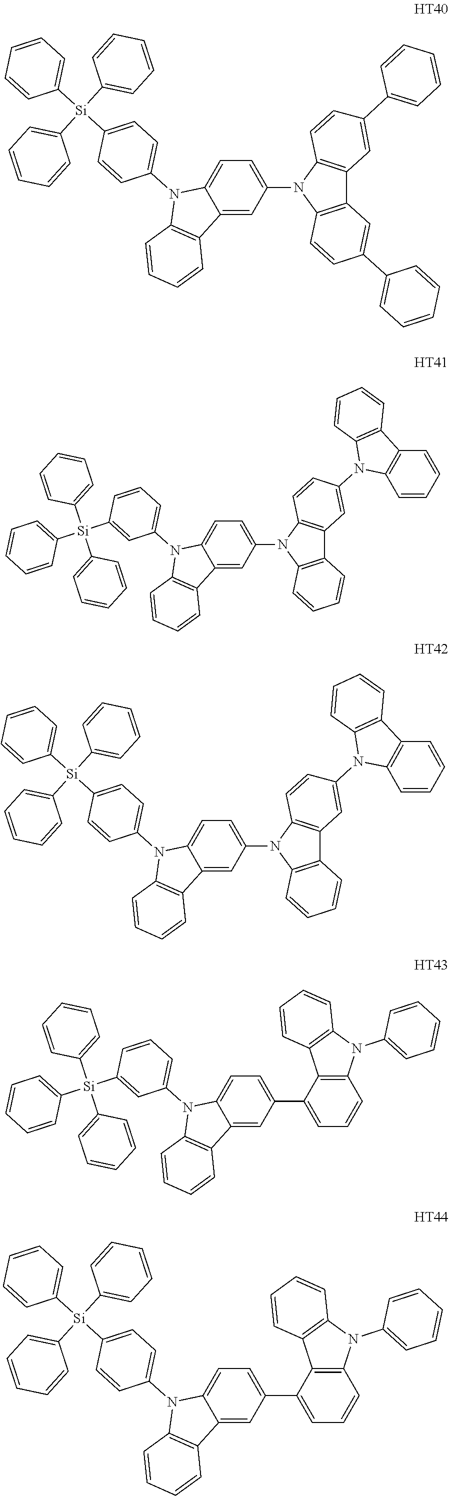

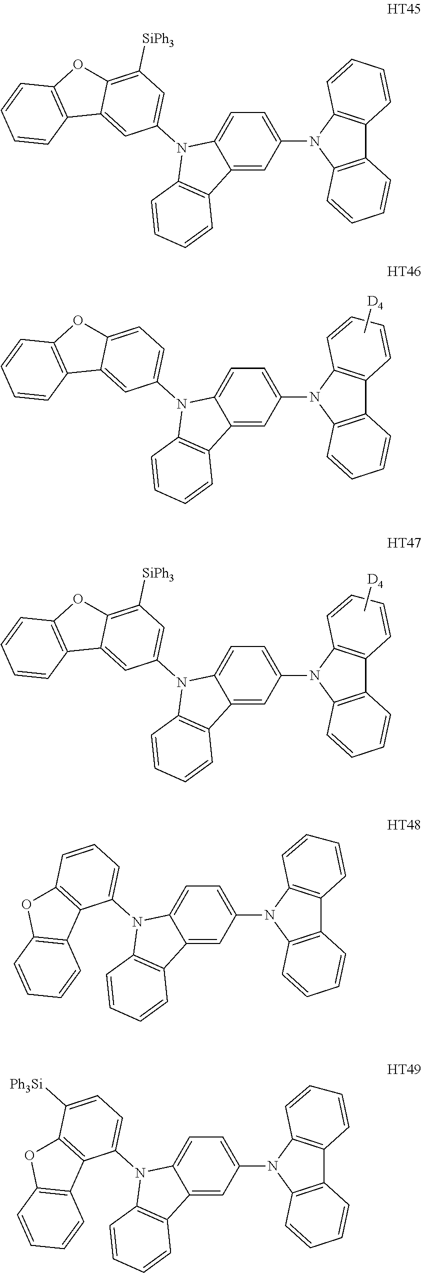

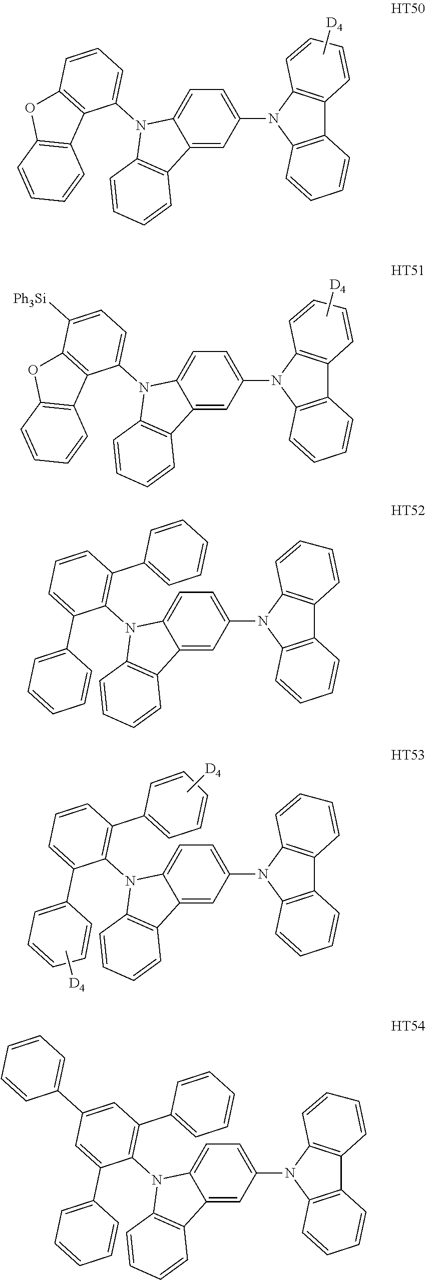

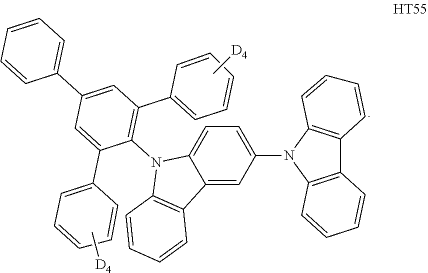

In one or more embodiments, the emission layer EML may include a third compound represented by Formula ET-1. The third compound represented by Formula ET-1 may include a heterocycle including N as a ring-forming atom. For example, the third compound may be used as the electron transport host material of the emission layer EML. In some embodiments, the third compound may be used as the electron transport material of the electron transport region ETR.

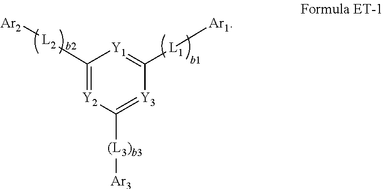

In Formula ET-1, at least one selected from Y₁ to Y₃ may be N, and the remainder may be CRₐ, and Rₐ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

b1 to b3 may be each independently an integer of 0 to 10. L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. If any of b1 to b3 are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₁ to Ar₃ may be substituted or unsubstituted phenyl groups, or substituted or unsubstituted carbazole groups.

The third compound may be represented by any one selected from compounds in Compound Group 3 below. The light emitting element ED of one or more embodiments may include any one selected from the compounds in Compound Group 3. In Compound Group 3, D is a deuterium atom, and Ph is a phenyl group.

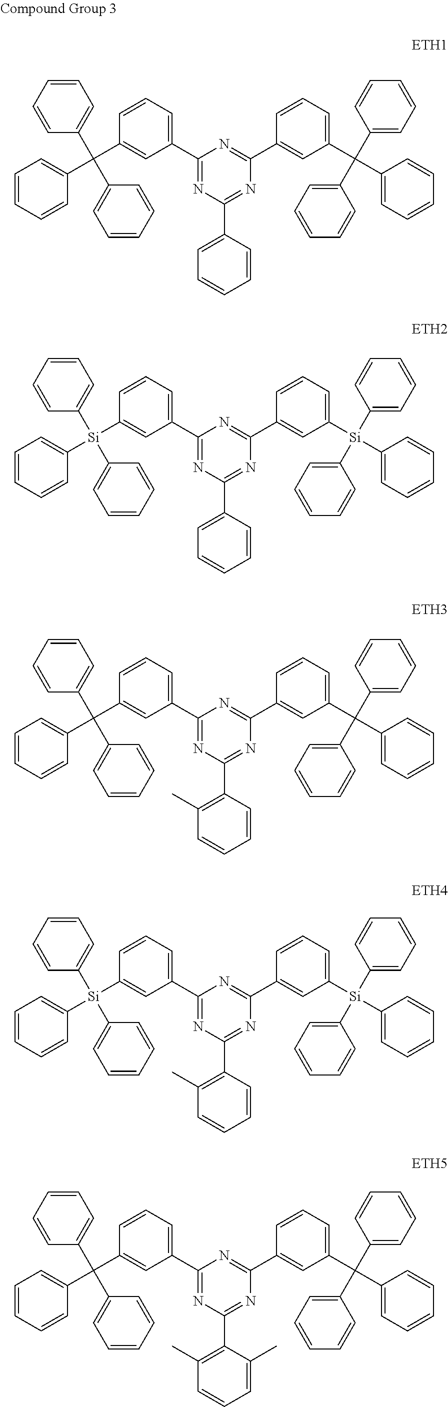

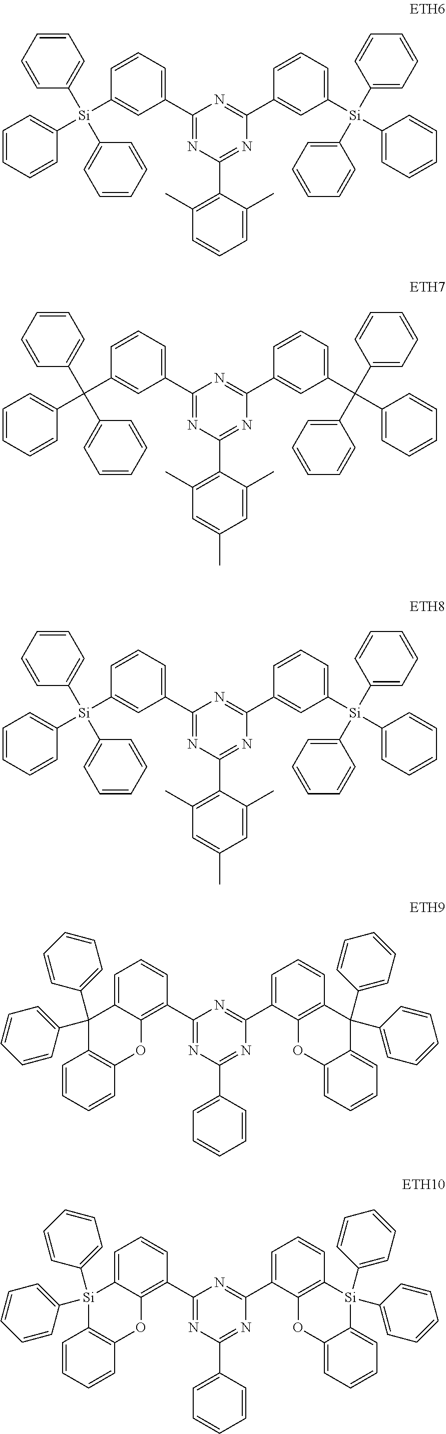

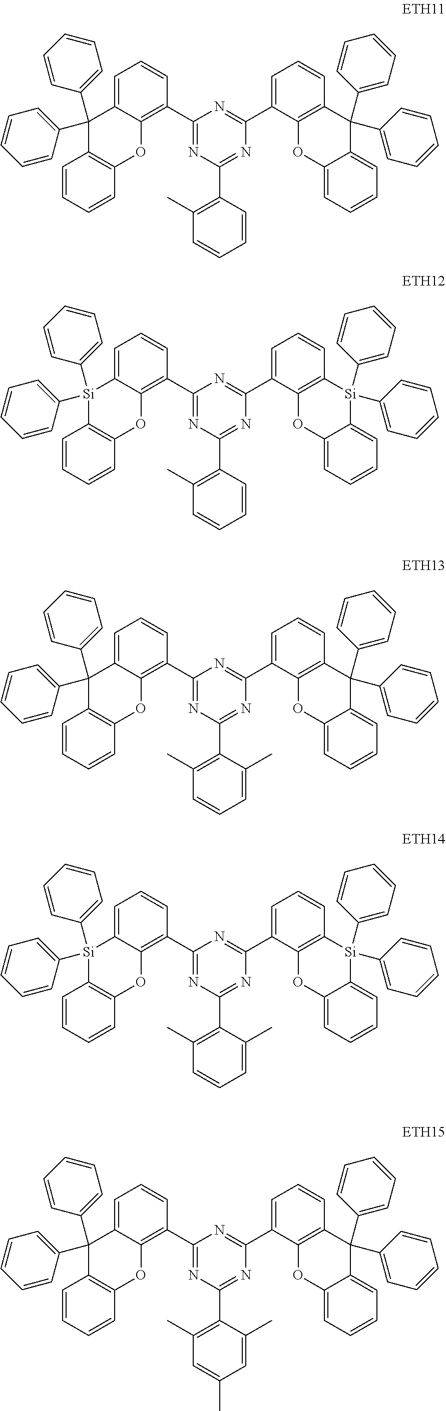

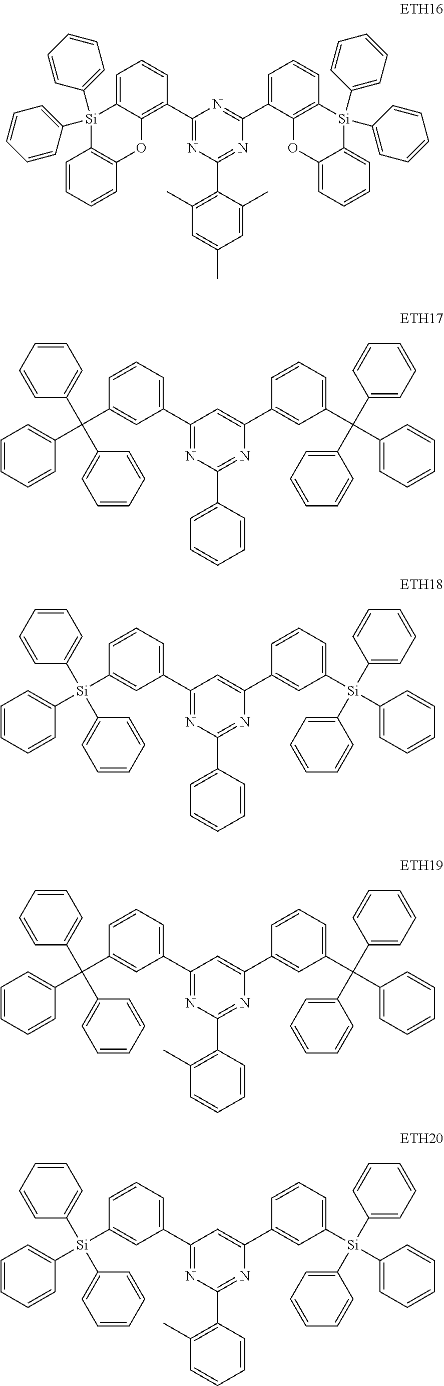

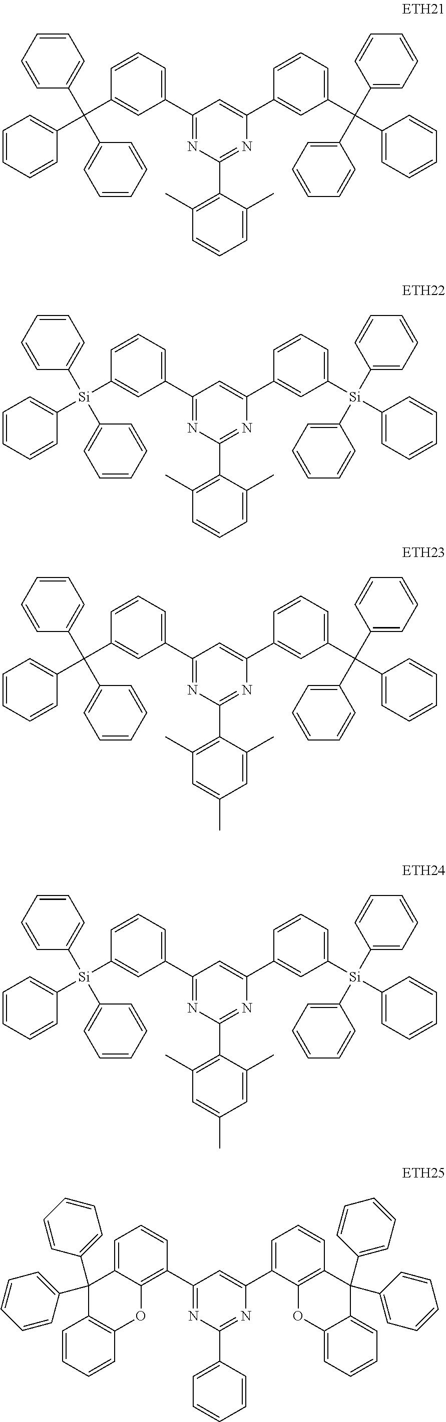

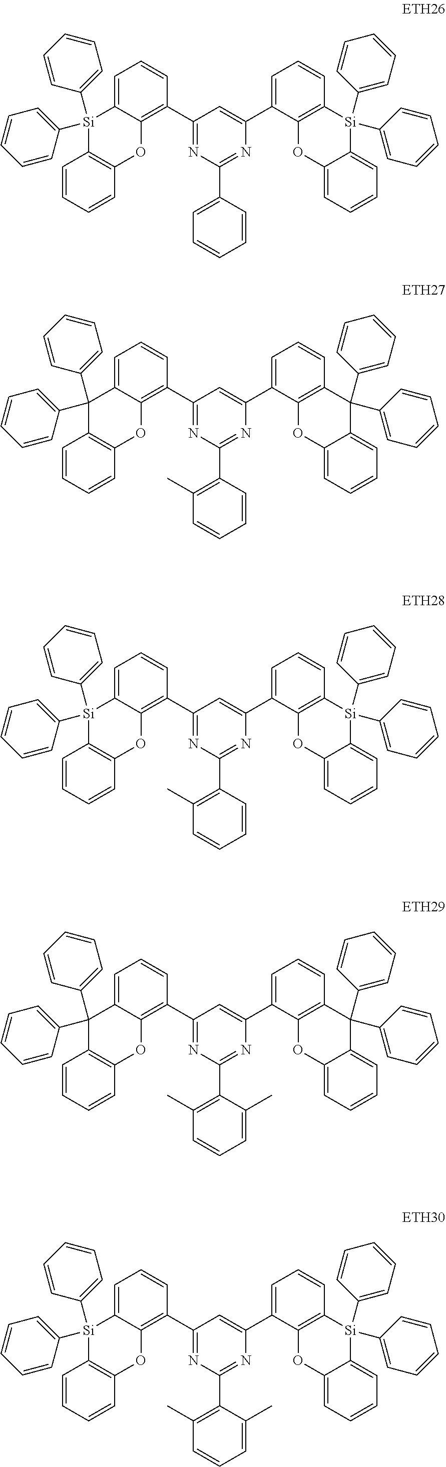

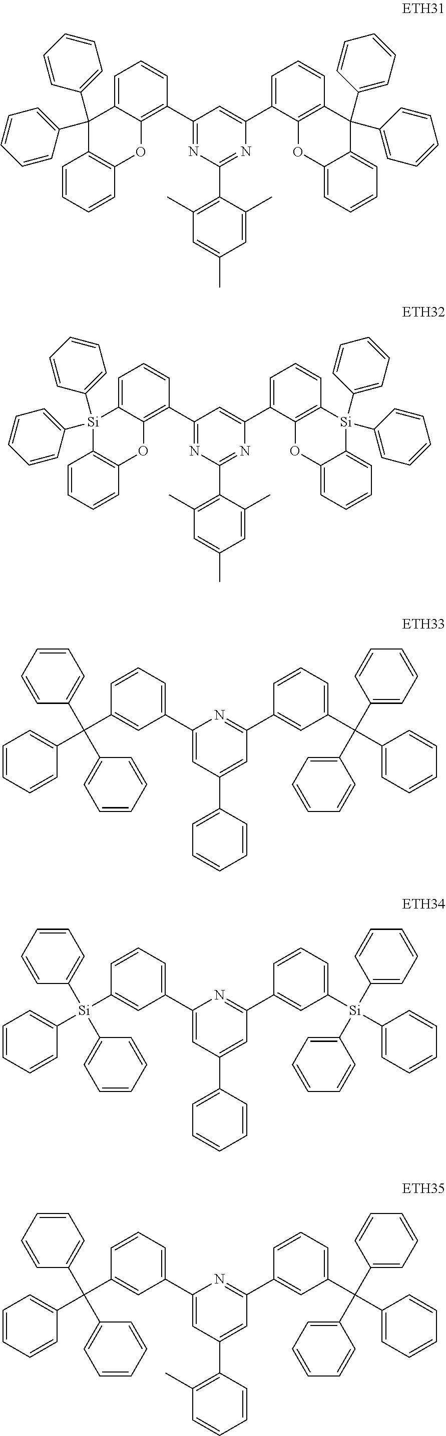

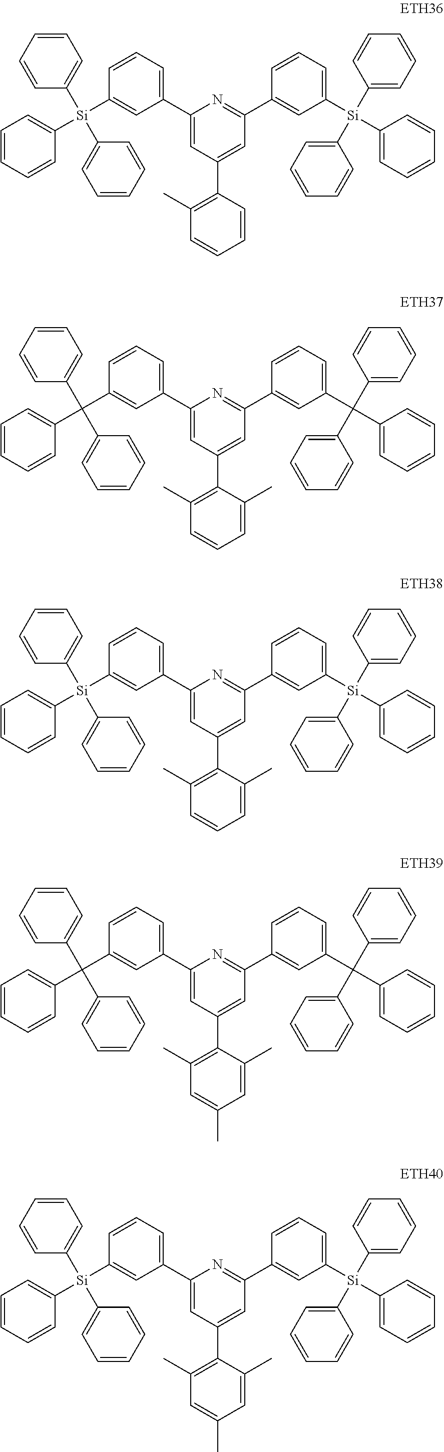

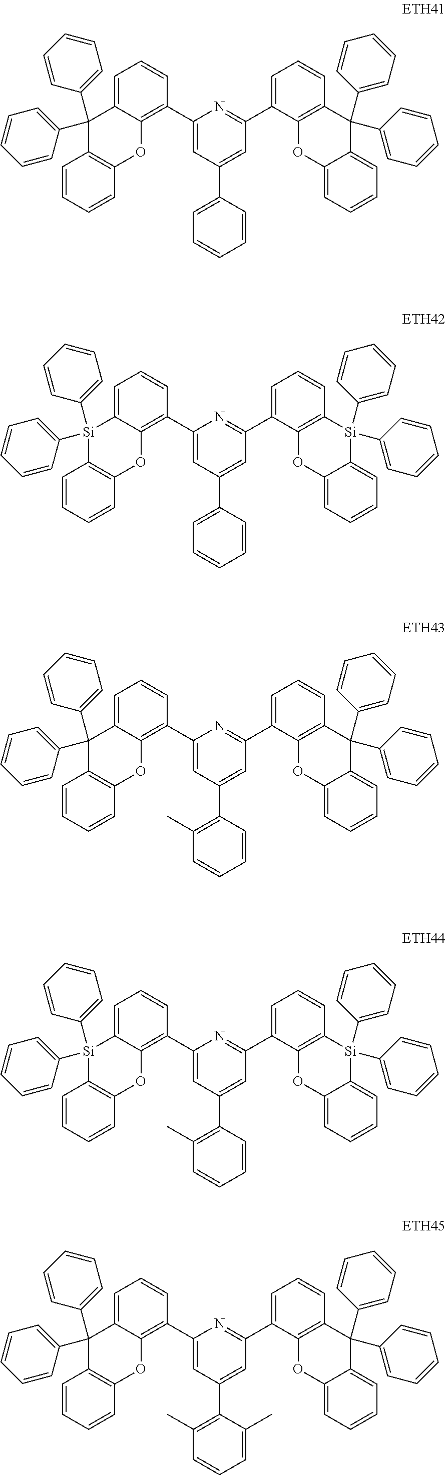

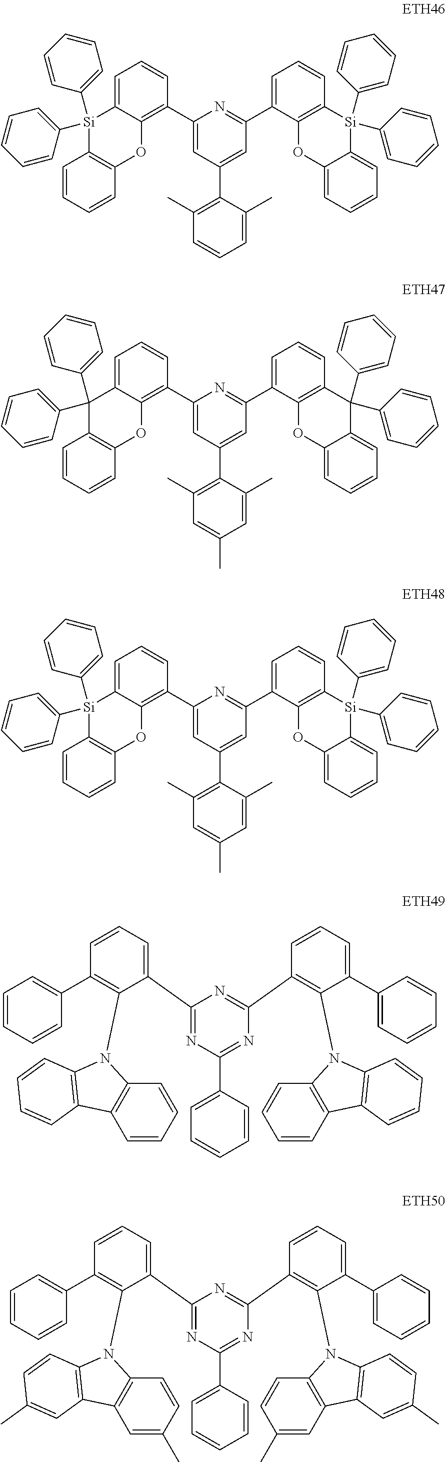

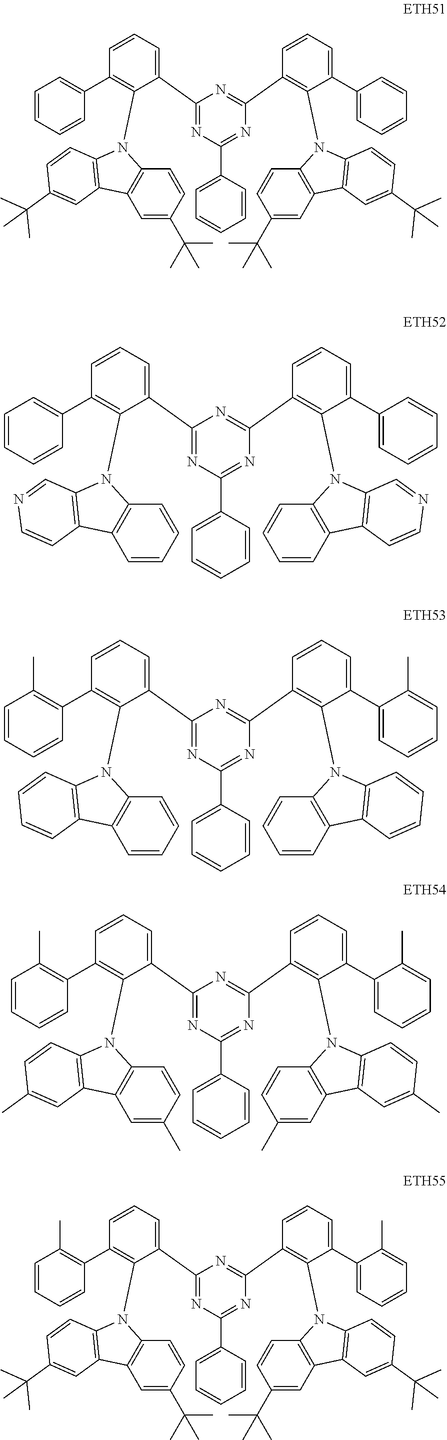

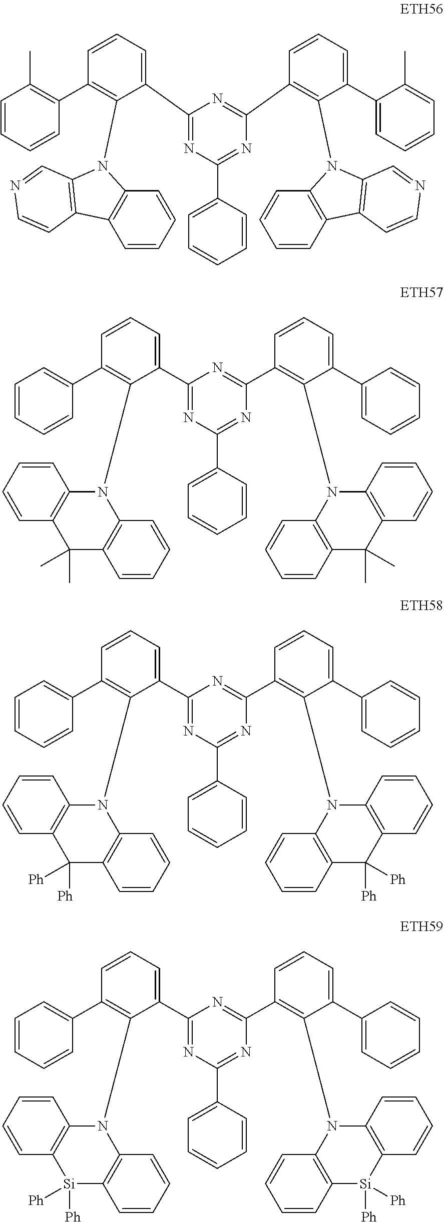

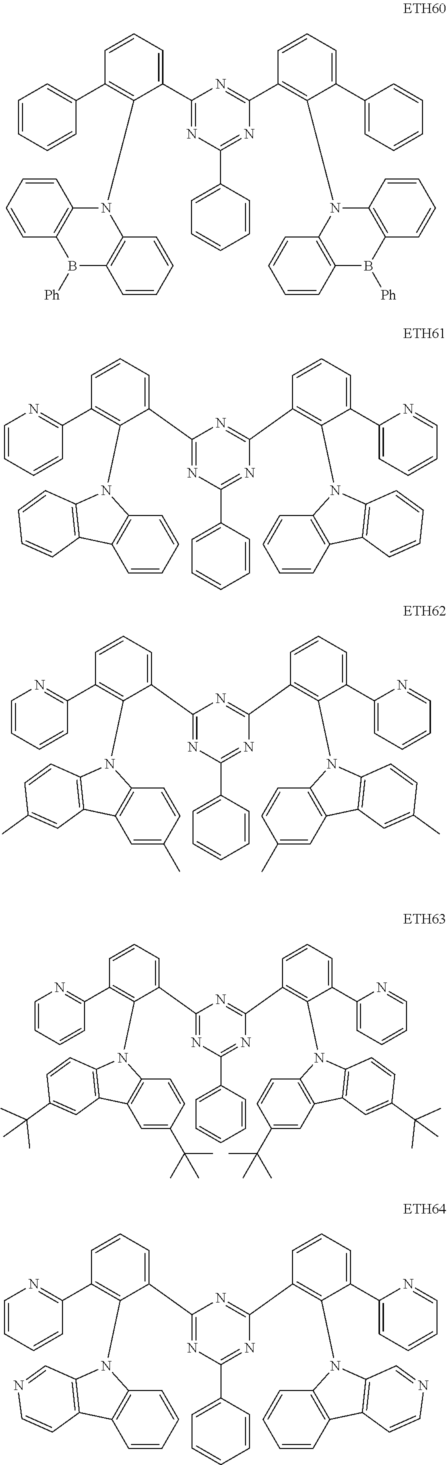

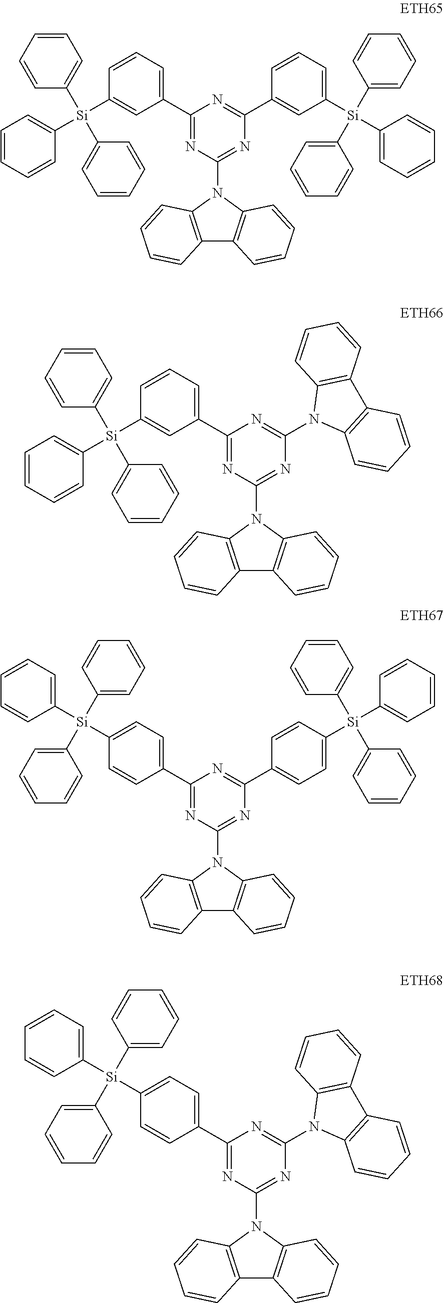 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

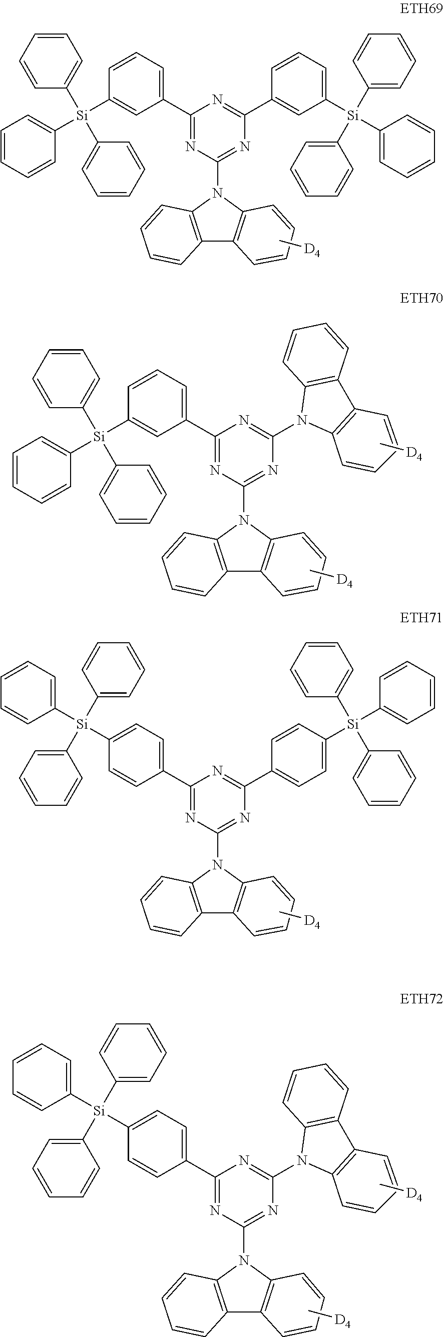

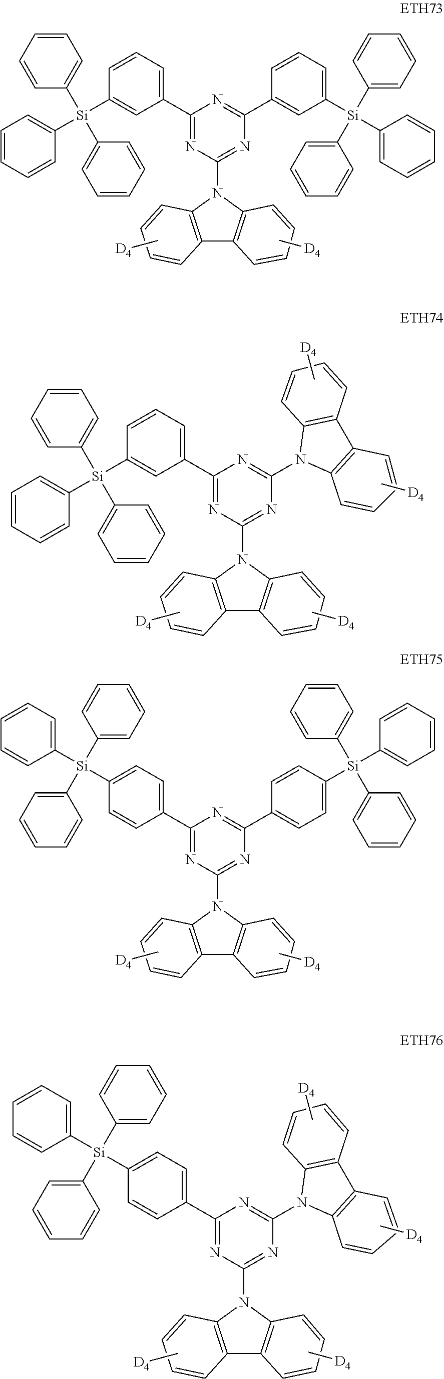

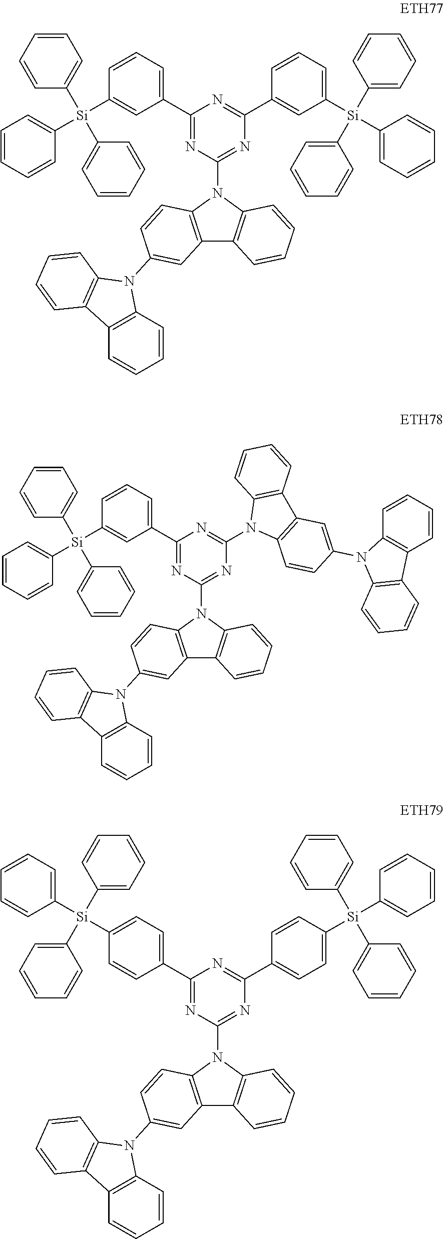 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

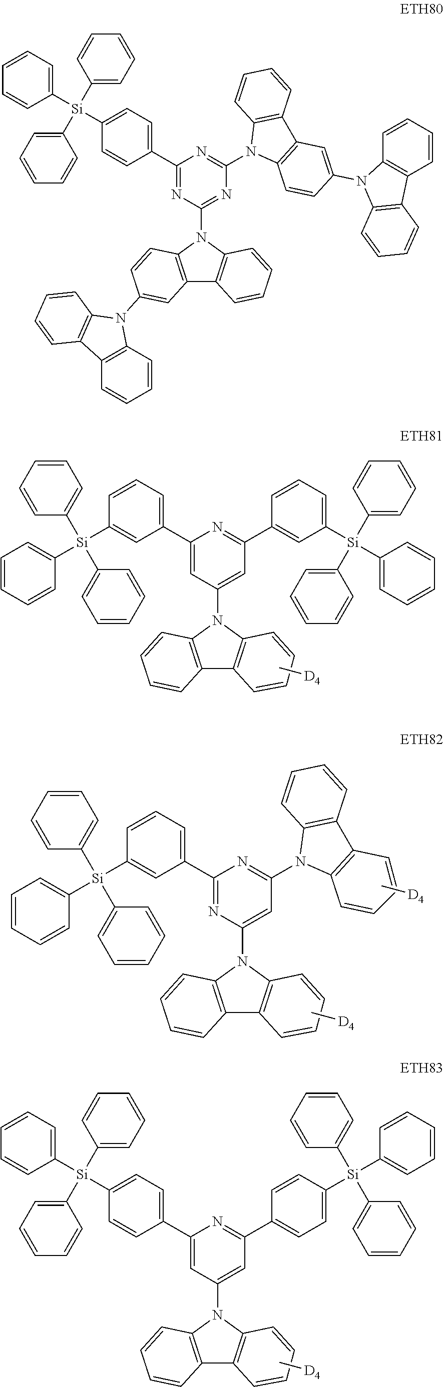

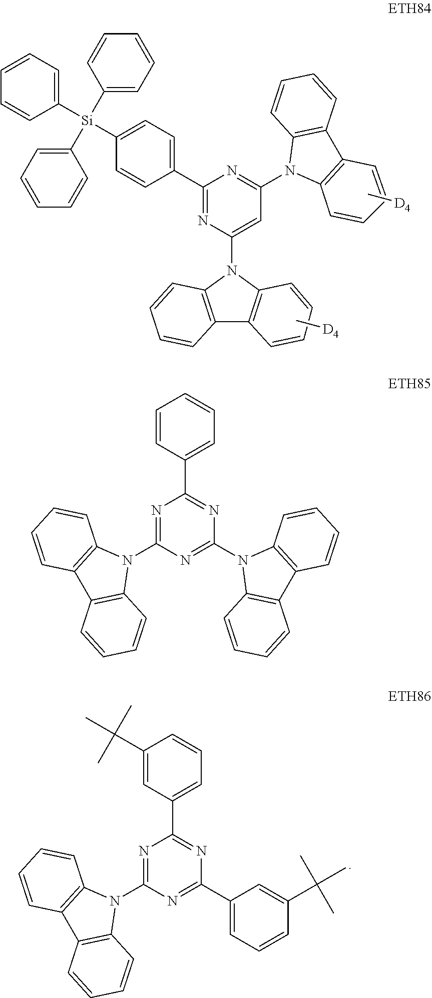

In one or more embodiments, the emission layer EML may include a fourth compound represented by Formula M-b below. The fourth compound represented by Formula M-b may include platinum (Pt) as a central metal. For example, the fourth compound may be used as a phosphorescence sensitizer of the emission layer EMIL. In some embodiments, the fourth compound may be used as the phosphorescence dopant material of the emission layer EMIL.

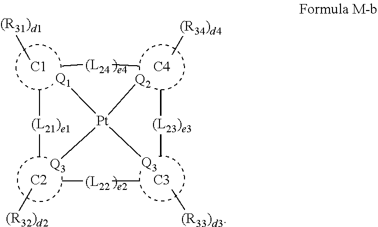

In Formula M-b, Q₁ to Q₄ may be each independently C or N, C1 to C4 may be each independently a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may be each independently a direct linkage, *—O—*, *—S—*,

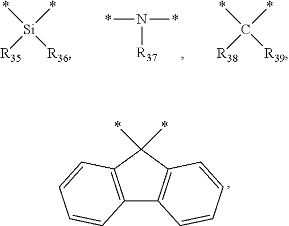

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may be each independently 0 or 1.

In Formula M-b, R₃₁ to R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may be each independently an integer of 0 to 4.

The compound represented by Formula M-b may be used as a blue phosphorescence dopant or a green phosphorescence dopant. The compound represented by Formula M-b may be represented by any one selected from compounds below. However, the compounds below are illustrations, and the compound represented by Formula M-b is not limited to the compounds represented below.

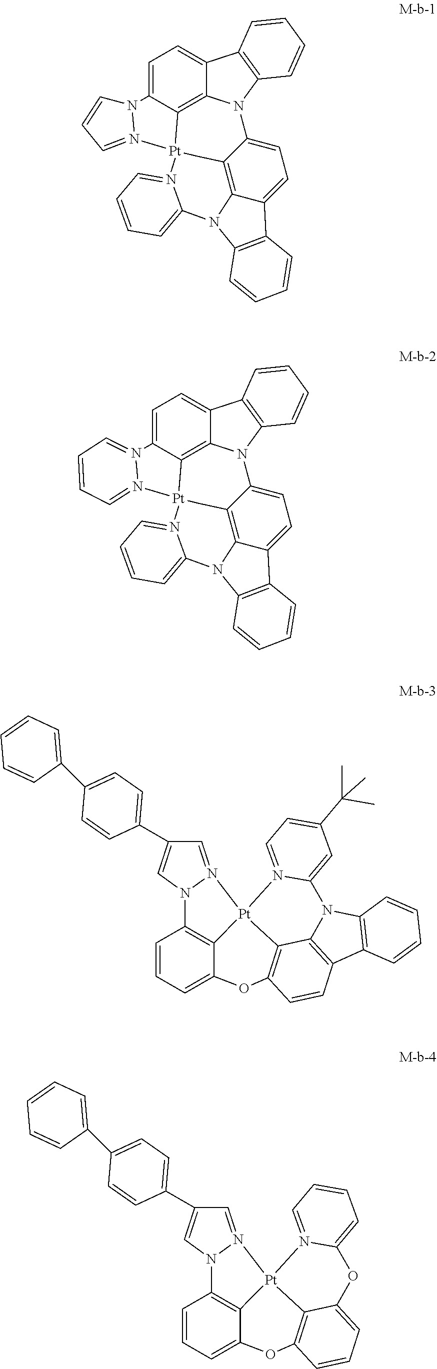

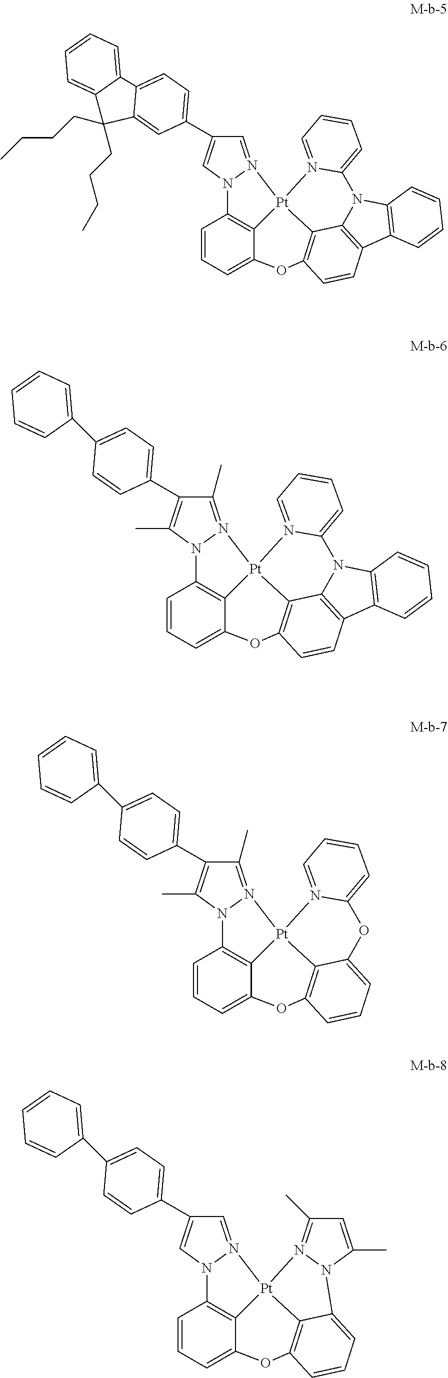

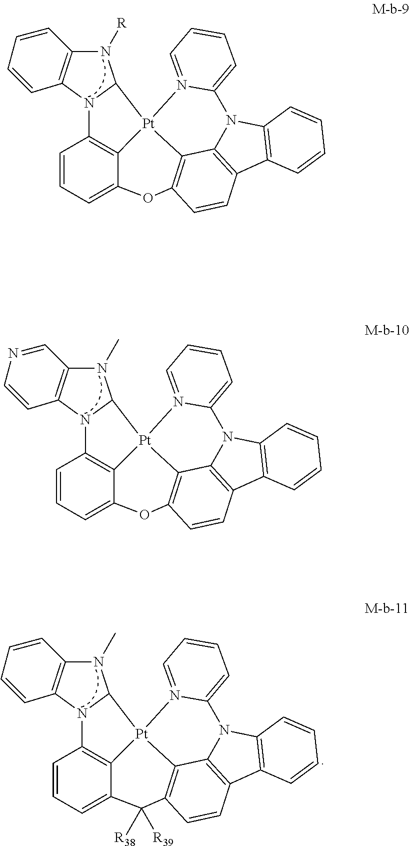

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include a compound represented by Formula M-a below. The compound represented by Formula M-a may be used as a phosphorescence dopant material.

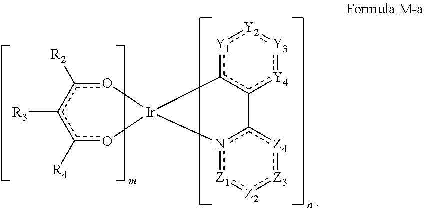

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be used as a phosphorescence dopant. The compound represented by Formula M-a may be represented by any one selected from Compounds M-a1 to M-a25 below. However, Compounds M-a1 to M-a25 are illustrations, and the compound represented by Formula M-a is not limited to those represented by Compounds M-a1 to M-a25.

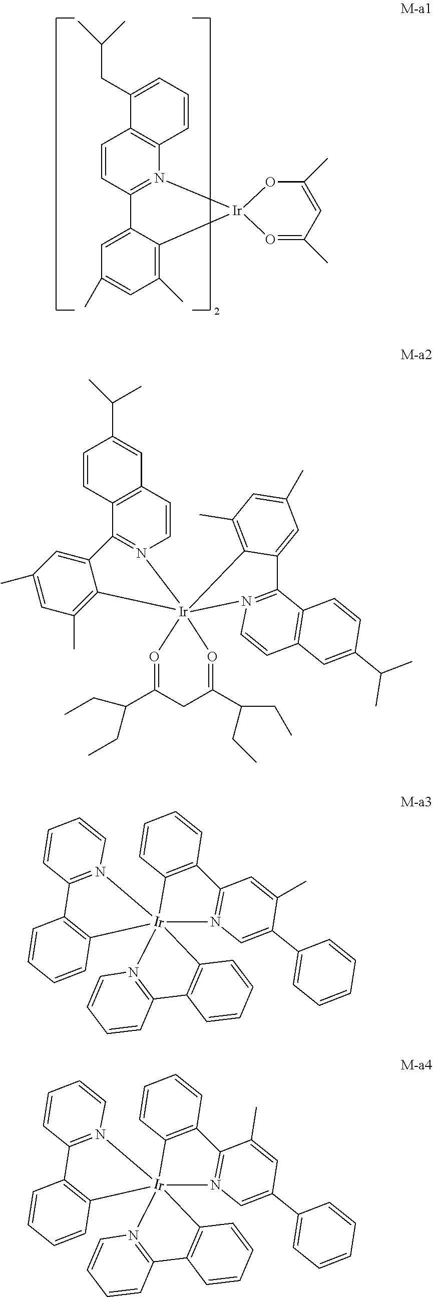

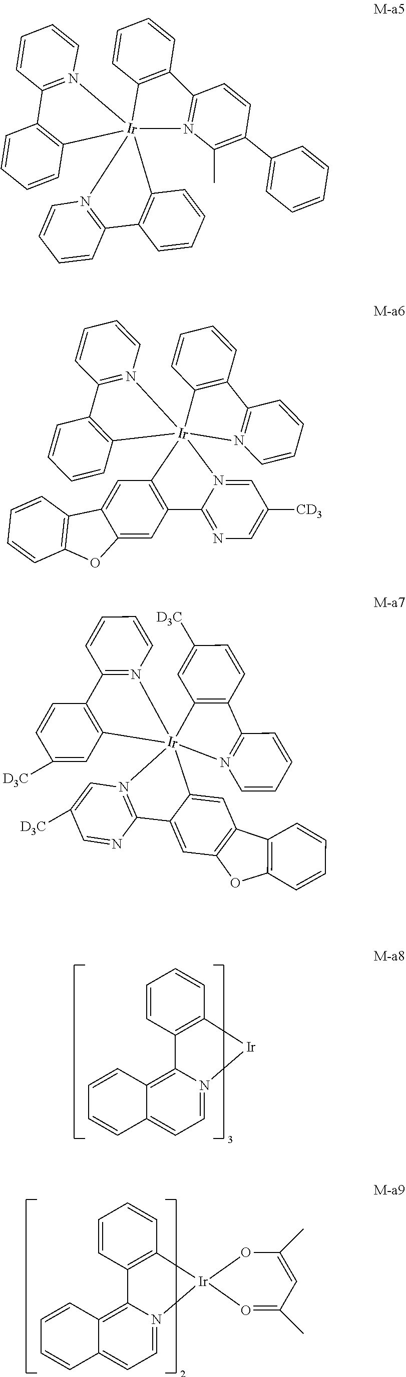

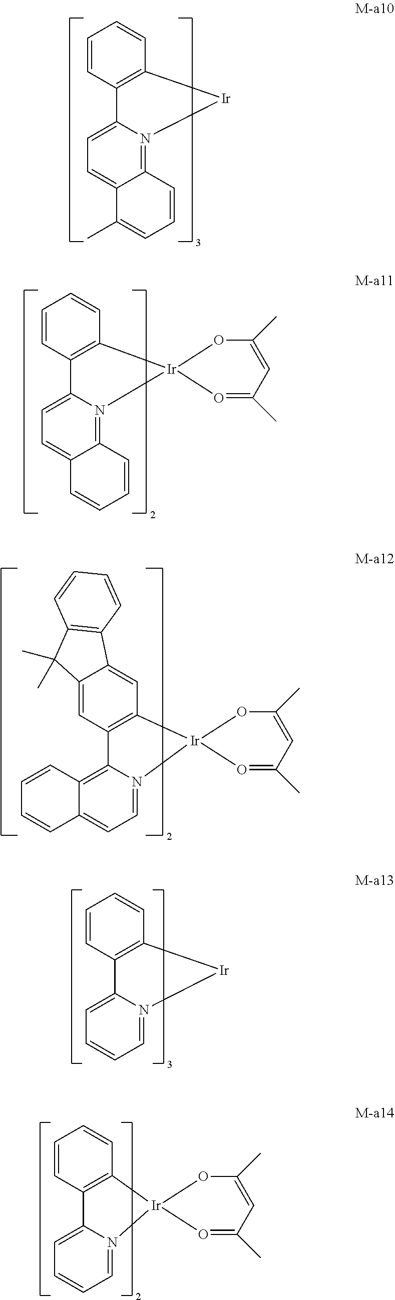

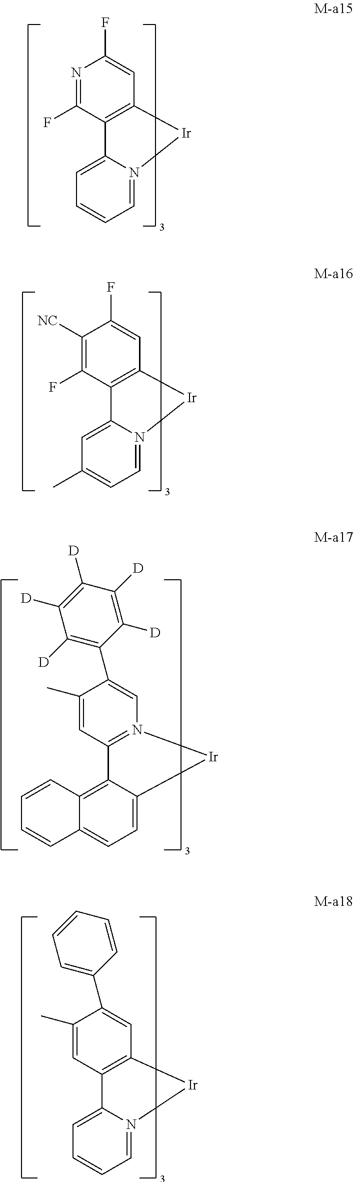

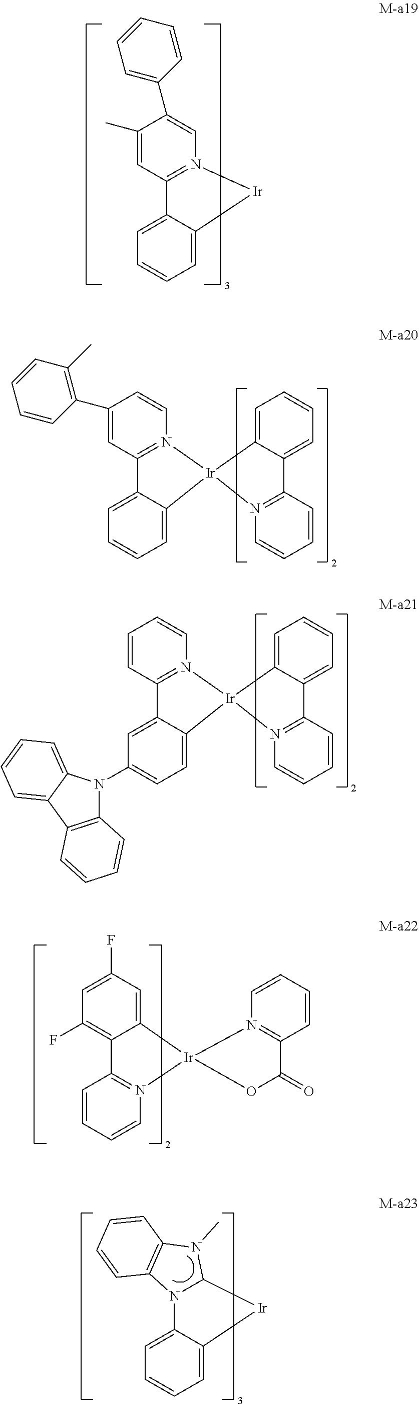

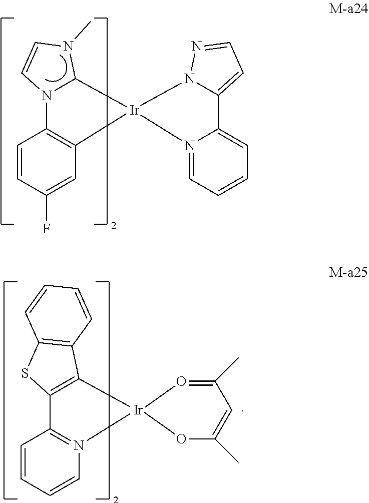

Compound M-a1 and Compound M-a2 may be used as red dopant materials, and Compound M-a3 to Compound M-a7 may be used as green dopant materials.

In one or more embodiments, the emission layer EML may include a compound represented by Formula E-2a or Formula E-2b below. The compound represented by Formula E-2a or Formula E-2b below may be used as a phosphorescence host material.

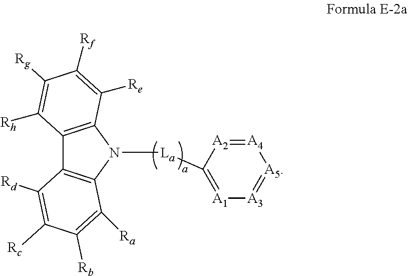

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. If “a” is an integer of 2 or more, multiple La may be each independently a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may be each independently N or CRi. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom. In Formula E-2a, two or three selected from A₁ to A₅ may be N, and the remainder may be CRI.

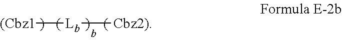

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from compounds in Compound Group E-2 below. However, the compounds listed in Compound Group E-2 below are only illustrations, and the compound represented by Formula E-2a or Formula E-2b is not limited to the compounds represented in Compound Group E-2 below.

### Compound Group E-2

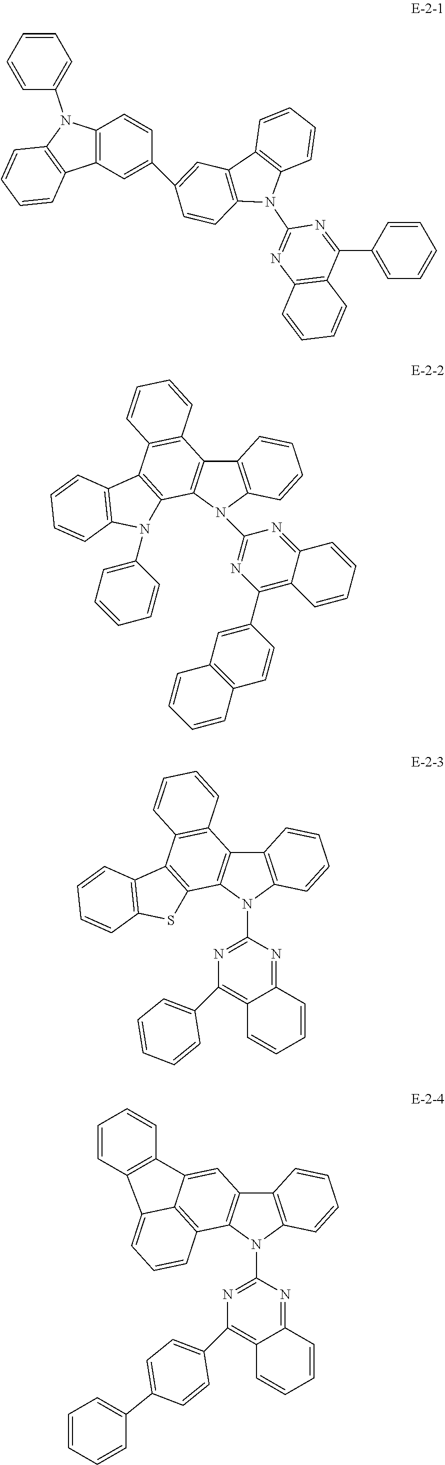

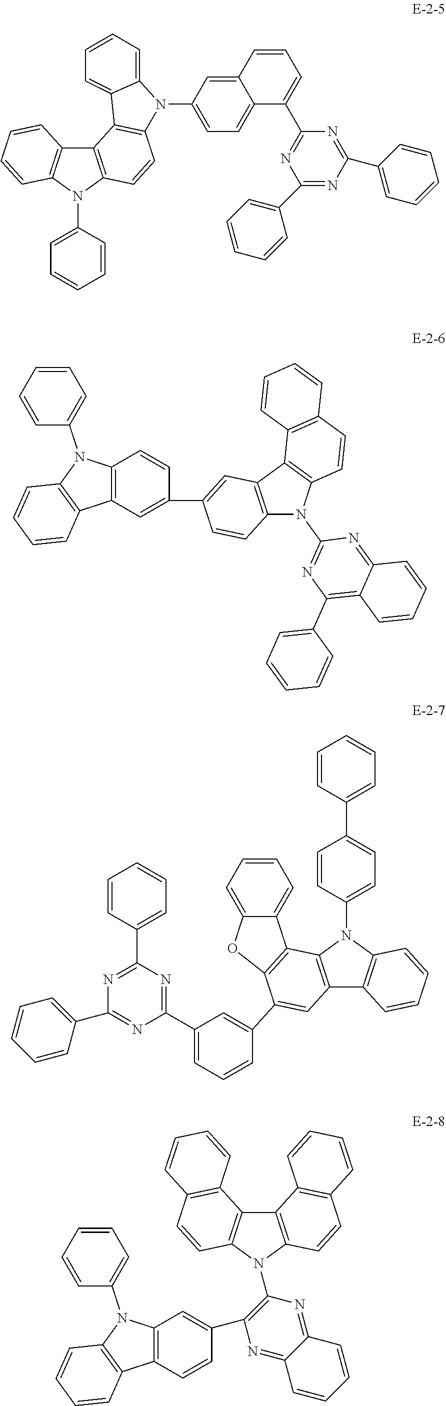 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

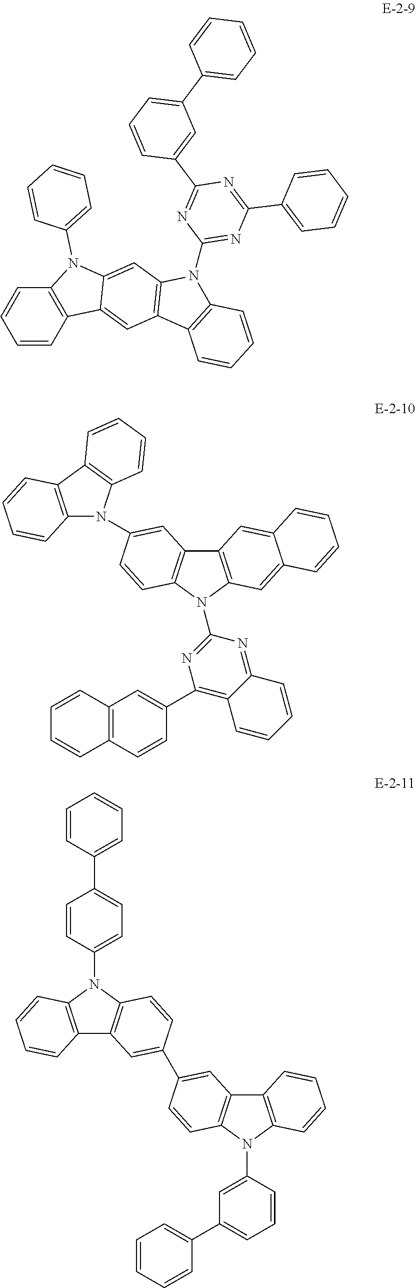

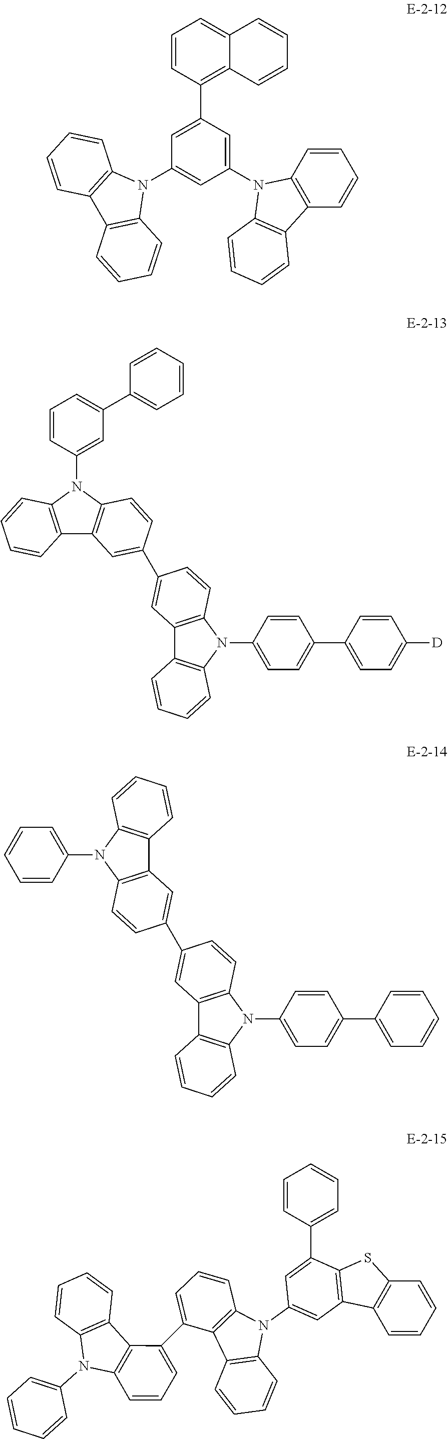 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

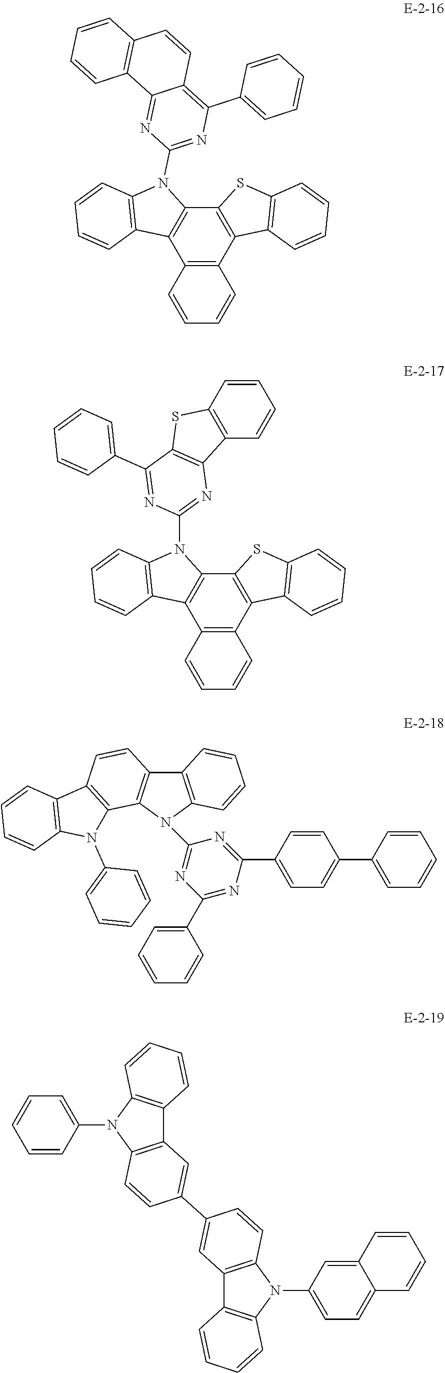 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

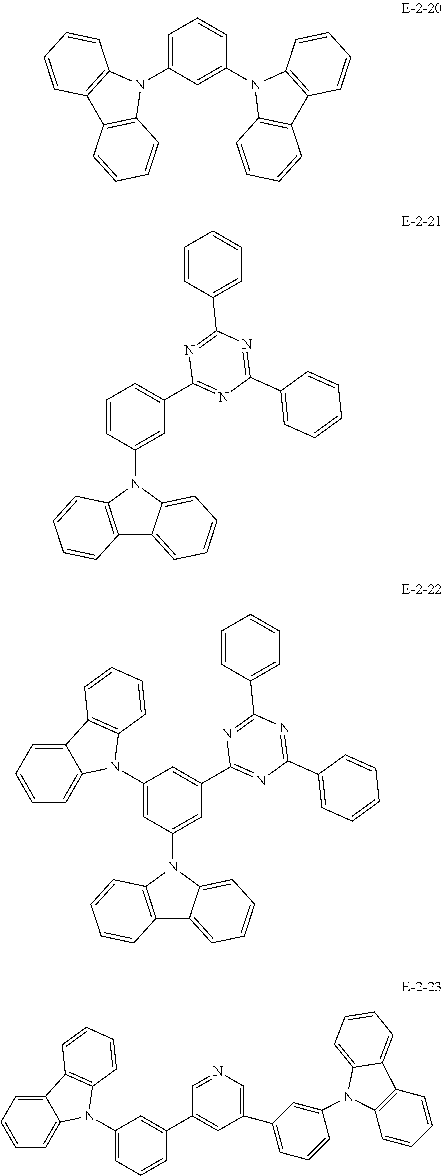 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

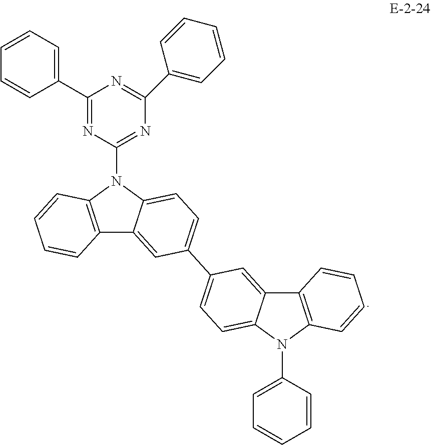 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a suitable host material. For example, the emission layer EML may include, as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may further include a compound represented by any one selected from Formula F-a to Formula F-c below. The compounds represented by Formula F-a to Formula F-c may be used as fluorescence dopant materials.

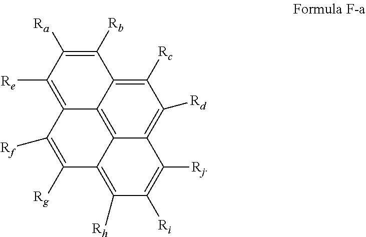

In Formula F-a, two selected from Rₐ to Rⱼ may be each independently substituted with *—NAr₁Ar₂. The remainder not substituted with *—NAr₁Ar₂ selected from Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one selected from Ar₁ and Ar₂ may be a heteroaryl group including O or S as a ring-forming atom.

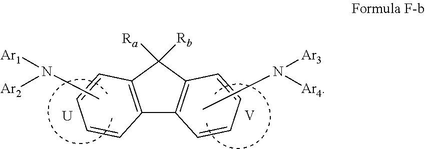

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms. In Formula F-b, the number of rings represented by U and V may be each independently 0 or 1.

For example, in Formula F-b, if the number of U or V is 1, one ring forms a fused ring at the designated part by U or V, and if the number of U or V is 0, a ring is not present at the designated part by U or V. For example, if the number of U is 0, and the number of V is 1, or if the number of U is 1, and the number of V is 0, a fused ring having the fluorene core of Formula F-b may be a ring compound with four rings. In some embodiments, if the number of both U and V is 0, the fused ring having a fluorene core of Formula F-b may be a ring compound with three rings. In some embodiments, if the number of both U and V is 1, a fused ring having the fluorene core of Formula F-b may be a ring compound with five rings.

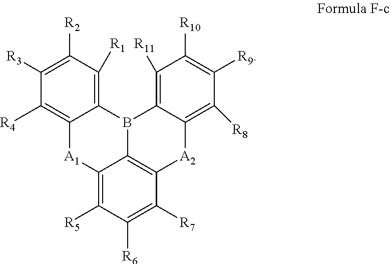

In Formula F-c, A₁ and A₂ may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may be each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may be each independently combined with the substituents of an adjacent ring to form a fused ring. For example, if A₁ and A₂ may be each independently NRₘ, A₁ may be combined with R₄ or R₅ to form a ring. In some embodiments, A₂ may be combined with R₇ or R₈ to form a ring.

In one or more embodiments, the emission layer EML may include a suitable dopant material, for example, one of more styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and/or one of more of the derivatives thereof (for example, 2,5,8,11-tetra-t-butylperylene (TBP)), pyrene and/or one of more of the derivatives thereof (for example, 1,1-dipyrene, 1,4-dipyrenylbenzene, and/or 1,4-bis(N,N-diphenylamino)pyrene), etc.

The emission layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), and/or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), and/or platinum octaethyl porphyrin (PtOEP) may be used as the phosphorescence dopant. However, one or more embodiments of the present disclosure is not limited thereto.

The emission layer EML may include a quantum dot material. The core of the quantum dot may be selected from II-VI group compounds, III-VI group compounds, I-III-VI group compounds, III-V group compounds, III-II-V group compounds, IV-VI group compounds, IV group elements, IV group compounds, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of, CdZnSeS, CdZnSeTe, CdZnSTe, CdHgSeS, CdHgSeTe, CdHgSTe, HgZnSeS, HgZnSeTe, HgZnSTe, and mixtures thereof.

The III-VI group compound may include a binary compound such as In₂S₃ and/or In₂Se₃, a ternary compound such as InGaS₃ and/or InGaSe₃, or optional combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, and a quaternary compound such as AgInGaS₂ and/or CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. The III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In this case, the binary compound, the ternary compound and/or the quaternary compound may be present at substantially uniform concentration in a particle or may be present at a partially different concentration distribution state in the same particle. In some embodiments, a core/shell structure in which one quantum dot wraps another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal oxide, a non-metal oxide, a semiconductor compound, and combinations thereof.

For example, the metal oxide and the non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, or a ternary compound such as MgAl₂O₄, CoFe₂O₄, NiFe₂O₄ and/or CoMn₂O₄, but one or more embodiments of the present disclosure is not limited thereto.

In one or more embodiments, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but one or more embodiments of the present disclosure is not limited thereto.

The quantum dot may have a full width of half maximum (FWHM) of emission wavelength spectrum of about 45 nm or less, for example, about 40 nm or less, or about 30 nm or less. Within any of these ranges, color purity and/or color reproducibility may be improved. In some embodiments, light emitted via such quantum dot is emitted in all directions, and light view angle properties may be improved.

In some embodiments, the shape of the quantum dot may be any suitable shape in the art, without specific limitation. For example, the shape of spherical, pyramidal, multi-arm, and/or cubic nanoparticle, nanotube, nanowire, nanofiber, nanoplate, etc. may be used.

The quantum dot may control the color of light emitted according to the particle size, and accordingly, the quantum dot may have various emission colors such as blue, red and green.

In the light emitting elements ED of embodiments, as shown in FIG. 3 to FIG. 7, the electron transport region ETR is provided on the emission layer EML. The electron transport region ETR may include at least one of a hole blocking layer HBL, an electron transport layer ETL or an electron injection layer EIL. However, one or more embodiments of the present disclosure is not limited thereto.

The electron transport region ETR may have a single layer formed using a single material, a single layer formed using multiple different materials, or a multilayer structure having multiple layers formed using multiple different materials. For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-2.

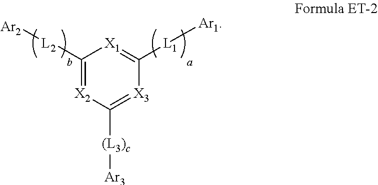

In Formula ET-2, at least one selected from X₁ to X₃ is N, and the remainder are CRₐ. Rₐ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may be each independently an integer of 0 to 10. In Formula ET-2, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. if “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include the above-described third compound represented by Formula ET-1 above. The electron transport region ETR may include an anthracene-based compound. However, one or more embodiments of the present disclosure is not limited thereto. The electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1, 10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), and/or mixture(s) thereof, without limitation.

The electron transport region ETR may include at least one selected from Compounds ET1 to ET36.

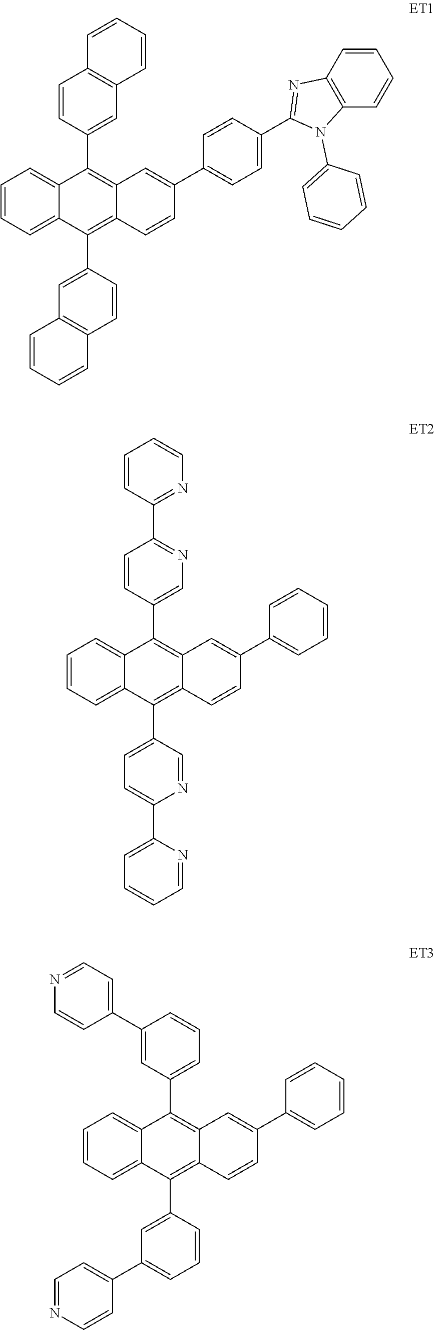 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

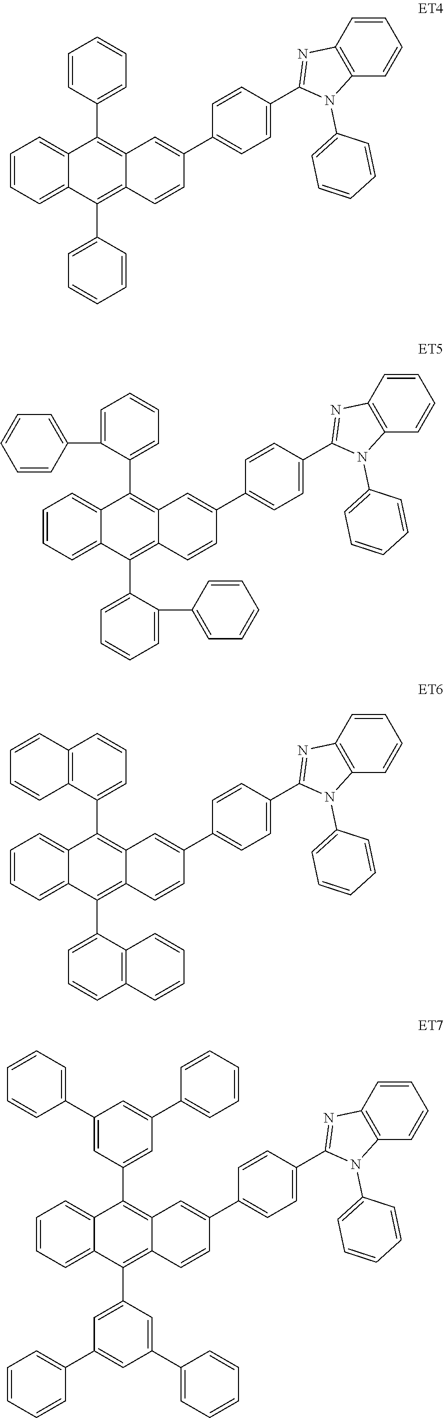 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

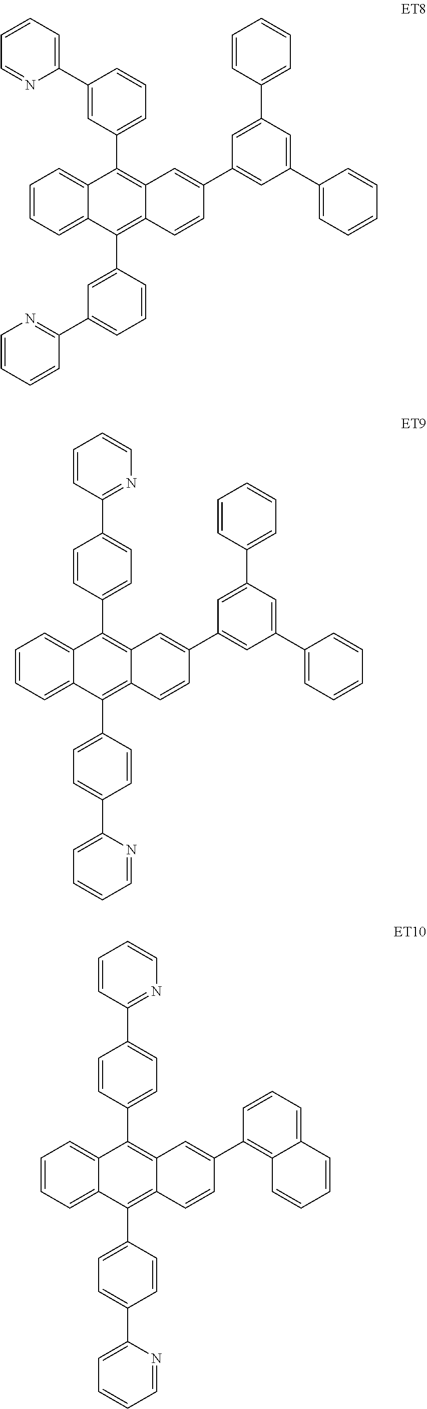 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

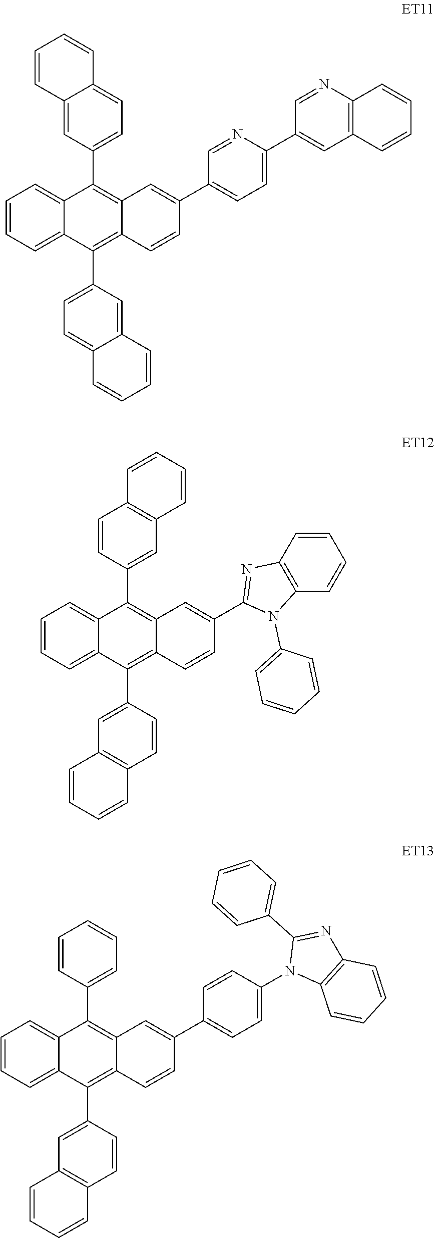 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

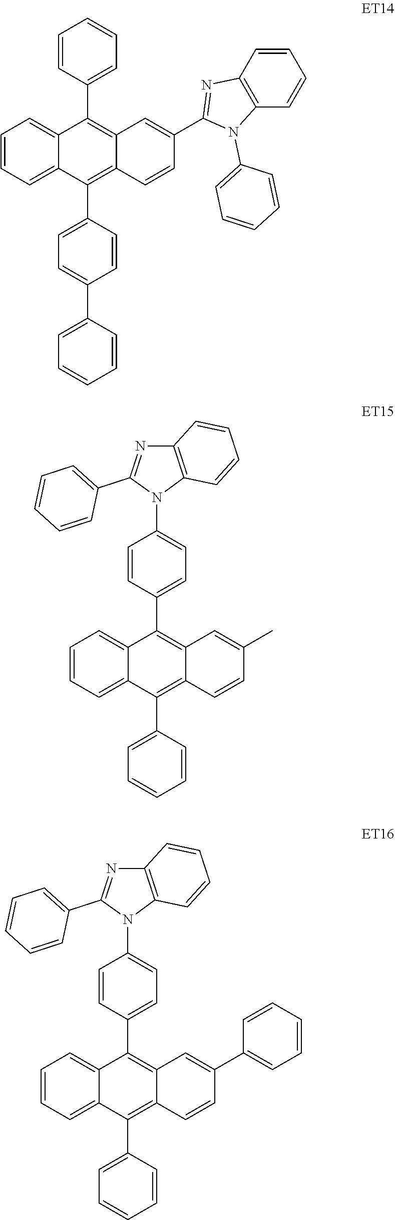 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

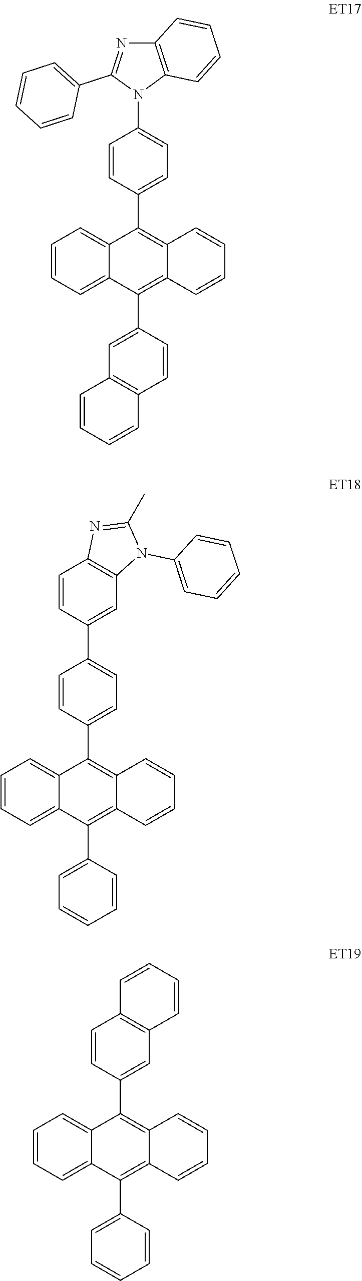

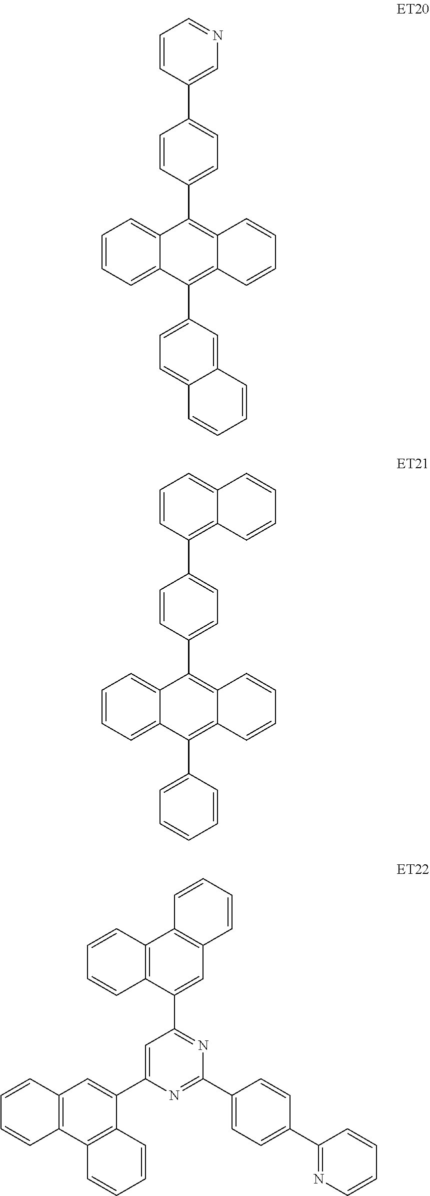

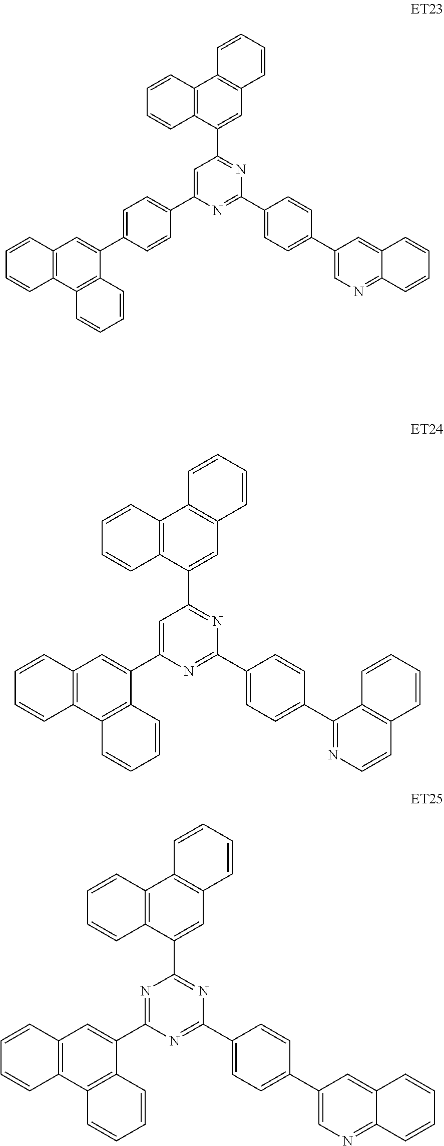 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

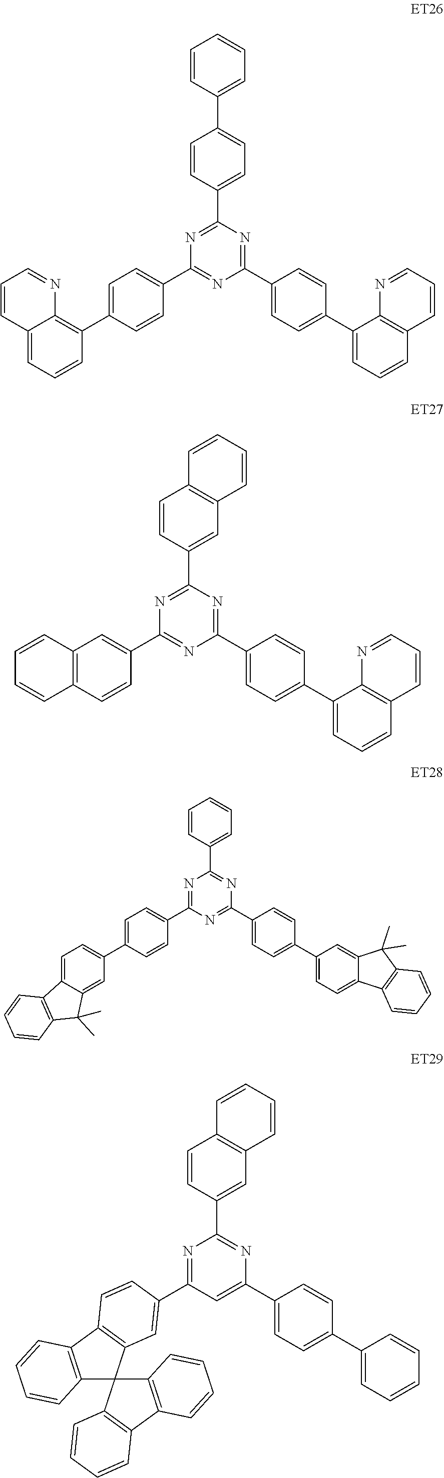 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

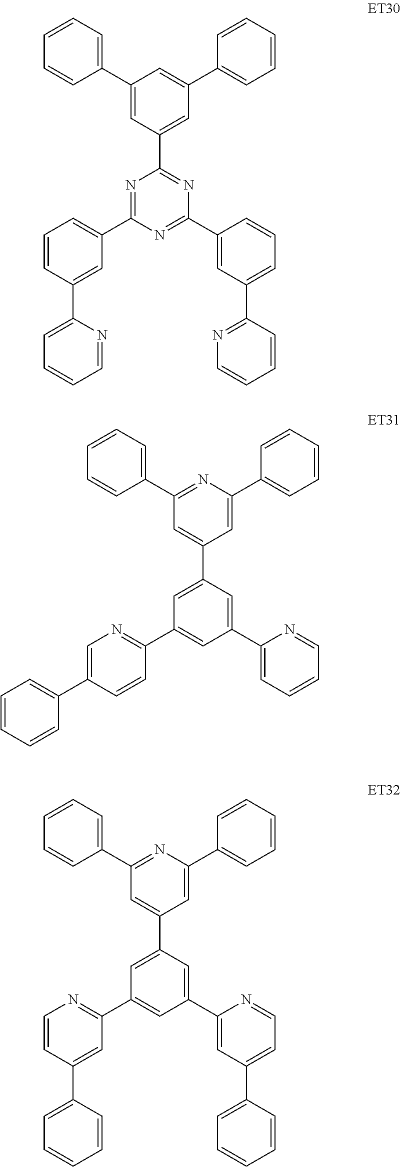 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

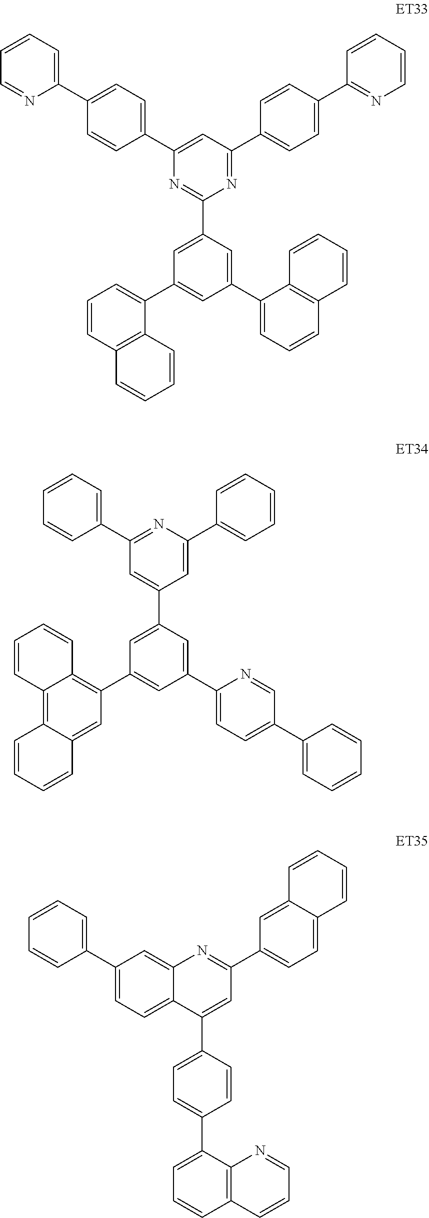 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

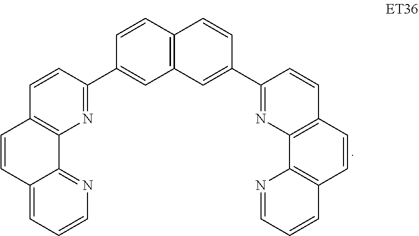 [SMILES: c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI and/or KI, a metal in lanthanoides such as Yb, or a co-depositing material of the metal halide and the metal in lanthanoides. For example, the electron transport region ETR may include KI:Yb, RbI:Yb, LiF:Yb, etc., as the co-depositing material. The electron transport region ETR may use a metal oxide such as Li₂O and/or BaO, and/or 8-hydroxy-lithium quinolate (Liq). However, one or more embodiments of the present disclosure is not limited thereto. The electron transport region ETR also may be formed using a mixture material of an electron transport material and an insulating organo metal salt. The organo metal salt may be a material having an energy band gap of about 4 eV or more. For example, the organo metal salt may include, for example, metal acetate(s), metal benzoate(s), metal acetoacetate(s), metal acetylacetonate(s), and/or metal stearate(s).

The electron transport region ETR may include at least one of 2,9-dimethyl-4,7-diphenyl-1, 10-phenanthroline (BCP), diphenyl(4-(triphenylsilyl)phenyl)phosphine oxide (TSPO1) or 4,7-diphenyl-1, 10-phenanthroline (Bphen) in addition to the aforementioned materials. However, one or more embodiments of the present disclosure is not limited thereto.

The electron transport region ETR may include the compounds of the electron transport region ETR in at least one selected from an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies any of the above-described ranges, satisfactory or suitable electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies any of the above described ranges, satisfactory or suitable electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, if the first electrode EL1 is an anode, the second electrode EL2 may be a cathode, and if the first electrode EL1 is a cathode, the second electrode EL2 may be an anode. The second electrode EL2 may include at least one selected from Ag, Mg, Cu, Al, Pt, Pd, Au, Ni, Nd, Ir, Cr, Li, Ca, LiF, Mo, Ti, W, In, Sn, Zn, compounds of two or more selected from Ag, Mg, Cu, Al, Pt, Pd, Au, Ni, Nd, Ir, Cr, Li, Ca, LiF, Mo, Ti, W, In, Sn, and Zn, mixtures of two or more selected from Ag, Mg, Cu, Al, Pt, Pd, Au, Ni, Nd, Ir, Cr, Li, Ca, LiF, Mo, Ti, W, In, Sn, and Zn, and oxides of Ag, Mg, Cu, Al, Pt, Pd, Au, Ni, Nd, Ir, Cr, Li, Ca, LiF, Mo, Ti, W, In, Sn, and Zn.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds including thereof, or mixtures thereof (for example, AgMg, AgYb, or MgYb). In some embodiments, the second electrode EL2 may have a multilayered structure including a reflective layer or a transflective layer formed using any of the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, and/or oxides of the aforementioned metal materials.

In one or more embodiments, the second electrode EL2 may be connected with an auxiliary electrode. If the second electrode EL2 is connected with the auxiliary electrode, the resistance of the second electrode EL2 may decrease.

On the second electrode EL2 in the light emitting element ED of one or more embodiments, a capping layer CPL may be further provided. The capping layer CPL may include a multilayer or a single layer.

In one or more embodiments, the capping layer CPL may be an organic layer or an inorganic layer. For example, if the capping layer CPL includes an inorganic material, the inorganic material may include an alkali metal compound such as LiF, an alkaline earth metal compound such as SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., and/or may include an epoxy resin, and/or acrylate such as methacrylate. In some embodiments, a capping layer CPL may include at least one selected from Compounds P1 to P5 below, but one or more embodiments of the present disclosure is not limited thereto.

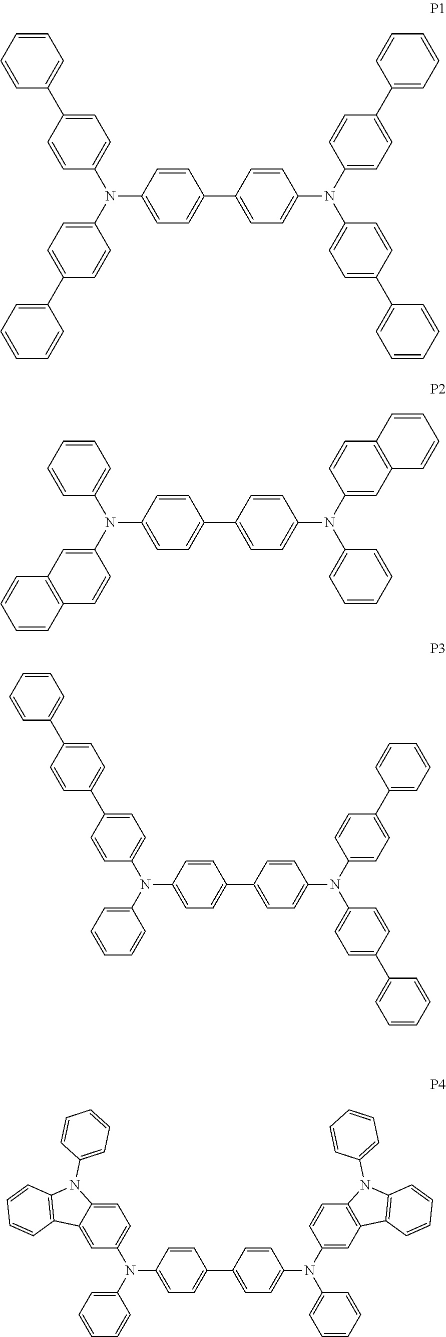 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

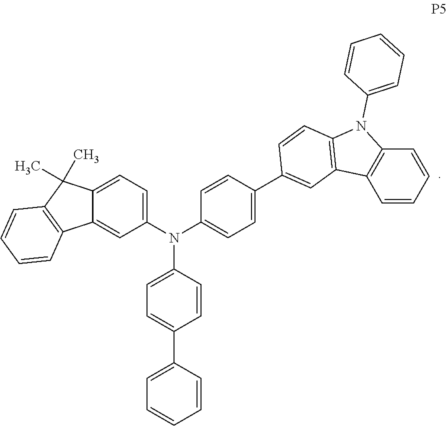 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

The refractive index of the capping layer CPL may be about 1.6 or more. For example, the refractive index of the capping layer CPL with respect to light in a wavelength range of about 550 nm to about 660 nm may be about 1.6 or more.

FIG. 8 to FIG. 11 are cross-sectional views on display devices according to embodiments. Hereinafter, in the explanation on the display devices of embodiments, referring to FIG. 8 to FIG. 11, the overlapping parts with the explanations already provided in connection with FIG. 1 to FIG. 7 will not be explained again, and the different features will be explained chiefly.

Referring to FIG. 8, a display device DD-a according to one or more embodiments may include a display panel DP including a display element layer DP-ED, a light controlling layer CCL provided on the display panel DP, and a color filter layer CFL. In one or more embodiments shown in FIG. 8, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. Descriptions of the structures of the light emitting elements of FIG. 3 to FIG. 7 may be applied to the structure of the light emitting element ED shown in FIG. 8. In one or more embodiments, the light emitting element ED may include the amine compound of one or more embodiments.

Referring to FIG. 8, the emission layer EML may be provided in an opening part OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display device DD-a of one or more embodiments, the emission layer EML may emit blue light. In one or more embodiments, the emission layer EML may be provided as a common layer for all luminous areas PXA-R, PXA-G and PXA-B.

The light controlling layer CCL may be provided on the display panel DP. The light controlling layer CCL may include a light converter. The light converter may be a quantum dot and/or a phosphor. The light converter may transform the wavelength of light provided and then emit the transformed light. For example, the light controlling layer CCL may be a layer including a quantum dot and/or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 8, a partition pattern BMP may be provided between the separated light controlling parts CCP1, CCP2 and CCP3, but one or more embodiments of the present disclosure is not limited thereto. In FIG. 8, the partition pattern BMP is shown not to be overlapped with the light controlling parts CCP1, CCP2 and CCP3, but at least a portion of the edge of the light controlling parts CCP1, CCP2 and/or CCP3 may be overlapped with the partition pattern BMP.

The light controlling layer CCL may include a first light controlling part CCP1 including a first quantum dot QD1 converting (e.g., configured to convert) first color light provided from the light emitting element ED into second color light, a second light controlling part CCP2 including a second quantum dot QD2 converting (e.g., configured to convert) first color light into third color light, and a third light controlling part CCP3 transmitting (e.g., configured to transmit) first color light. In one or more embodiments, the first light controlling part CCP1 may provide red light which is the second color light, and the second light controlling part CCP2 may provide green light which is the third color light. The third light controlling part CCP3 may transmit and provide blue light which is the first color light provided from the light emitting element ED. For example, the first quantum dot QD1 may be a red quantum dot, and the second quantum dot QD2 may be a green quantum dot. For the quantum dots QD1 and QD2, the same descriptions as those provided above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but may include the scatterer SP.

The scatterer SP may be an inorganic particle. For example, the scatterer SP may include at least one selected from TiO₂, ZnO, Al₂O₃, SiO₂, and hollow silica. The scatterer SP may include at least one selected from TiO₂, ZnO, Al₂O₃, SiO₂, and hollow silica, or may be a mixture of two or more materials selected from TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3, respectively, for dispersing the quantum dots QD1 and QD2 and the scatterer SP, respectively. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer SP dispersed in the third base resin BR3.

The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, will be referred to as “humidity/oxygen”). The barrier layer BFL1 may block or reduce the exposure of the light controlling parts CCP1, CCP2 and CCP3 to humidity/oxygen. The barrier layer BFL1 may cover the light controlling parts CCP1, CCP2 and CCP3. In some embodiments, the barrier layer BFL2 may be provided between filters CF1, CF2, and CF3 and the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride, and/or a metal thin film securing light transmittance. The barrier layers BFL1 and BFL2 may further include an organic layer. The barrier layers BFL1 and BFL2 may be composed of a single layer or multiple layers.

In the display device DD-a of one or more embodiments, the color filter layer CFL may be provided on the light controlling layer CCL. For example, the color filter layer CFL may be provided directly on the light controlling layer CCL. In this case, the barrier layer BFL2 may not be provided (e.g., may be omitted).

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. The first to third filters CF1, CF2 and CF3 may be provided corresponding to a red luminous area PXA-R, a green luminous area PXA-G and a blue luminous area PXA-B, respectively.

Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment or dye. one or more embodiments of the present disclosure is not limited thereto, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In some embodiments, the first filter CF1 and the second filter CF2 may be yellow filters. The first filter CF1 and the second filter CF2 may be provided in one body (e.g., integrally) without distinction.

In some embodiments, the color filter layer CFL may further include a light blocking part. The light blocking part may be a black matrix. The light blocking part may be formed by including an organic light blocking material or an inorganic light blocking material, including a black pigment and/or black dye. The light blocking part may prevent or reduce light leakage and may divide the boundaries among adjacent filters CF1, CF2 and CF3.

On the color filter layer CFL, a base substrate BL may be provided. The base substrate BL may be a member providing a base surface on which the color filter layer CFL, the light controlling layer CCL, etc. are provided. The base substrate BL may be a glass substrate, a metal substrate, a plastic substrate, etc. However, one or more embodiments of the present disclosure is not limited thereto, and the base substrate BL may be an inorganic layer, an organic layer, or a composite material layer. In some embodiments, the base substrate BL may not be provided (e.g., may be omitted).

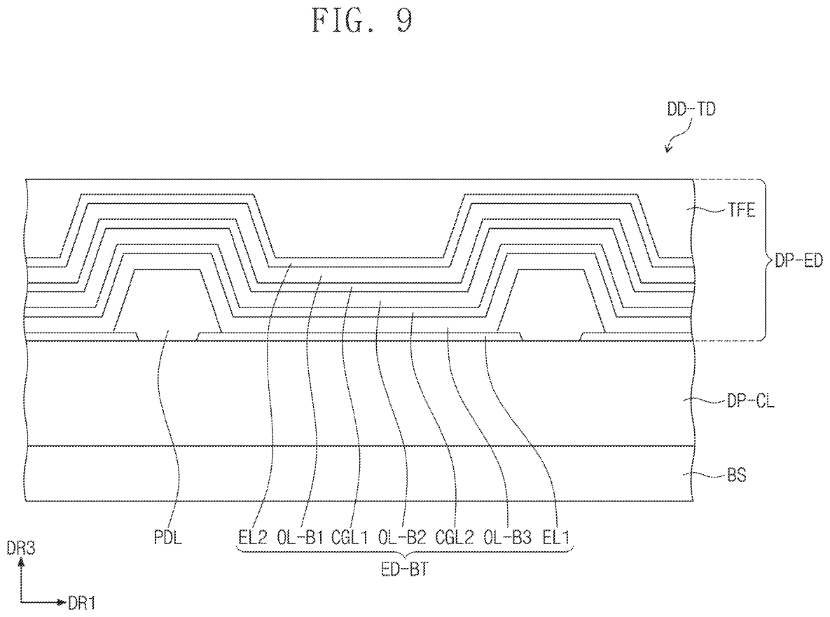

FIG. 9 is a cross-sectional view showing a part of the display device according to one or more embodiments. In FIG. 9, the cross-sectional view of a portion corresponding to the display panel DP in FIG. 8 is shown. In a display device DD-TD of one or more embodiments, the light emitting element ED-BT may include multiple light emitting structures OL-B1, OL-B2 and OL-B3. The light emitting element ED-BT may include oppositely provided first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 8), a hole transport region HTR (FIG. 8) and an electron transport region ETR (FIG. 8) provided with the emission layer EML (FIG. 8) therebetween. For example, the light emitting element ED-BT included in the display device DD-TD of one or more embodiments may be a light emitting element of a tandem structure including multiple emission layers. At least one selected from multiple light emitting structures OL-B1, OL-B2 and OL-B3 may include the amine compound of one or more embodiments.

In one or more embodiments shown in FIG. 9, light emitted from the light emitting structures OL-B1, OL-B2 and OL-B3 may be all blue light. However, one or more embodiments of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting (e.g., configured to emit) light in different wavelength regions may emit white light.

Between neighboring light emitting structures OL-B1, OL-B2 and OL-B3, charge generating layers CGL1 and CGL2 may be provided. The charge generating layers CGL1 and CGL2 may include a p-type charge generating layer (e.g., p charge generating layer) and/or an n-type charge generating layer (e.g., an n charge generating layer).

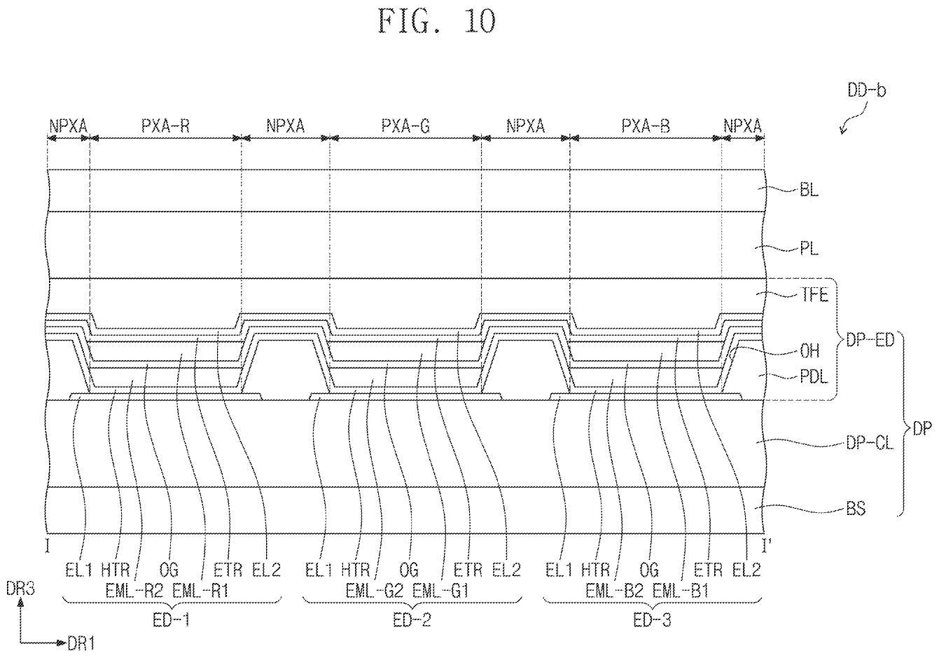

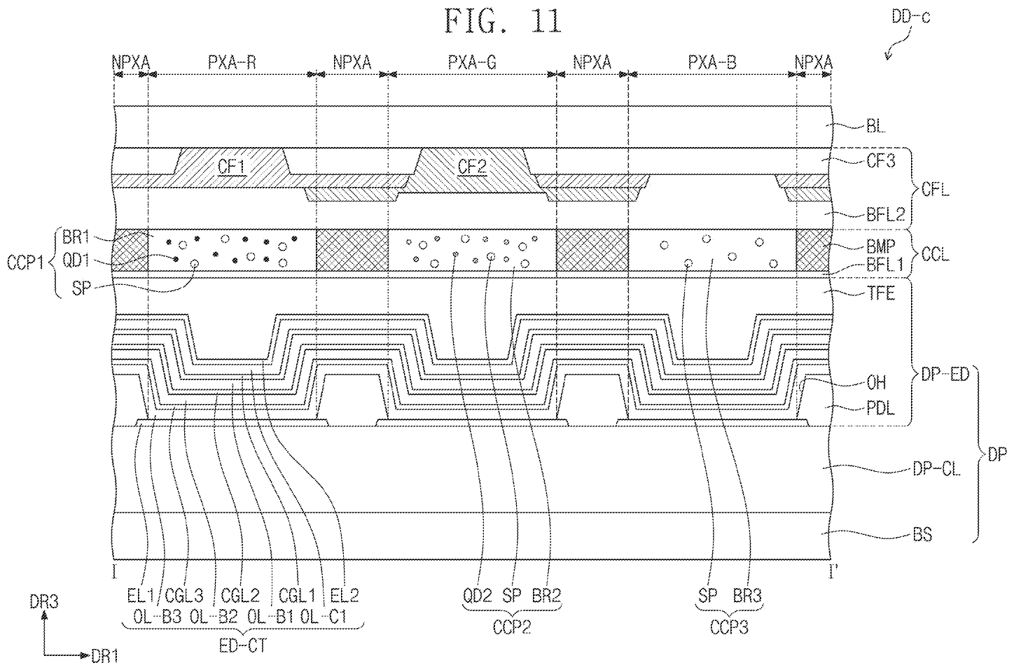

Referring to FIG. 10, a display device DD-b according to one or more embodiments may include light emitting elements ED-1, ED-2 and ED-3, formed by stacking two emission layers. Compared to the display device DD of one or more embodiments shown in FIG. 2, an embodiment shown in FIG. 10 is different in that first to third light emitting elements ED-1, ED-2 and ED-3 include two emission layers stacked in a thickness direction, each. In the first to third light emitting elements ED-1, ED-2 and ED-3, two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and/or between the first blue emission layer EML-B1 and the second blue emission layer EML-B2, an emission auxiliary part OG may be provided.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, one or more embodiments of the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be provided between the electron transport region ETR and the emission auxiliary part OG. The second red emission layer EML-R2, the second green emission layer EML-G2 and the second blue emission layer EML-B2 may be provided between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in this order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in this order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in this order.

An optical auxiliary layer PL may be provided on a display element layer DP-ED. The optical auxiliary layer PL may include a polarization layer. The optical auxiliary layer PL may be provided on a display panel DP and may control external light reflected at the display panel DP. In some embodiments, the optical auxiliary layer PL may not be provided (e.g., may be omitted) from the display device according to one or more embodiments.

Different from FIG. 9 and FIG. 10, a display device DD-c in FIG. 11 is shown to include four light emitting structures OL-B1, OL-B2, OL-B3 and OL-C1. A light emitting element ED-CT may include oppositely provided first electrode EL1 and second electrode EL2, and first to fourth light emitting structures OL-B1, OL-B2, OL-B3 and OL-C1 stacked in order in a thickness direction between the first electrode EL1 and the second electrode EL2. At least one selected from the first to fourth light emitting structures OL-B1, OL-B2, OL-B3 and OL-C1 may include the amine compound of one or more embodiments.

Between the first to fourth light emitting structures OL-B1, OL-B2, OL-B3 and OL-C1, charge generating layers CGL1, CGL2 and CGL3 may be provided. The charge generating layers CGL1, CGL2 and CGL3 provided between the neighboring light emitting structures OL-B1, OL-B2, OL-B3 and OL-C1 may include a p-type charge generating layer and/or an n-type charge generating layer.

Selected from the four light emitting structures, the first to third light emitting structures OL-B1, OL-B2 and OL-B3 may emit blue light, and the fourth light emitting structure OL-C1 may emit green light. However, one or more embodiments of the present disclosure is not limited thereto, and the first to fourth light emitting structures OL-B1, OL-B2, OL-B3 and OL-C1 may emit different wavelengths of light.

Hereinafter, referring to embodiments and comparative embodiments, the amine compound according to one or more embodiments and the light emitting element according to one or more embodiments of the present disclosure will be explained in more detail. However, the embodiments below are illustrations to assist the understanding of the present disclosure, and the scope of the present disclosure is not limited thereto.

### Examples

### 1. Synthesis of Amine Compounds of Embodiments

First, the synthetic methods of the amine compounds according to embodiments will be explained by illustrating the synthetic methods of Compounds 1, 2, 6, 14, 15, 16, 17, 24, 402, 422 and 451. However, the synthetic methods of the amine compounds explained hereinafter are embodiments, and the synthetic method of the amine compound according to one or more embodiments of the present disclosure is not limited to the embodiments below.

### (1) Synthesis of Compound 1

Amine Compound 1 according to one or more embodiments may be synthesized by, for example, the steps (e.g., the acts or the tasks) of Reaction 1 below.

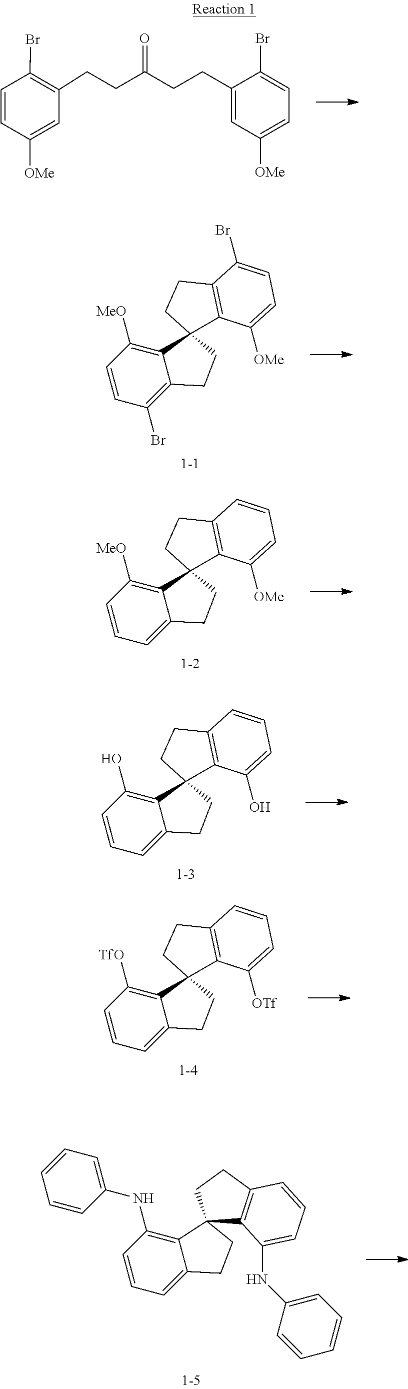

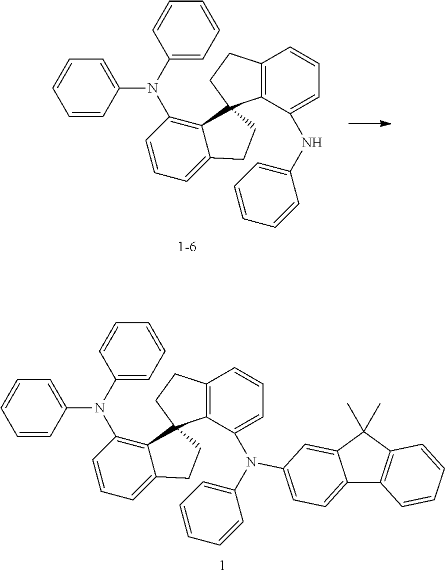

### 1) Synthesis of Intermediate Compound 1-1

To 1,5-bis(2-bromo-5-methoxyphenyl)pentan-3-one (50 mmol), polyphosphoric acid (150 g) was added and stirred at about 105° C. for about 6 hours. After cooling the reaction solution to room temperature, the reaction solution was added to 300 ml of water and extracted with dichloromethane three times. The organic layer thus obtained was dried over MgSO₄, and the residue obtained by vaporizing the solvent was purified and separated by silica gel chromatography to obtain Intermediate Compound 1-1 (yield 67%). The compound thus produced was identified through LC-MS (C₁₉H₁₈Br₂O₂:M+436.0).

### 2) Synthesis of Intermediate Compound 1-2

To Intermediate Compound 1-1 (30 ml), tetrahydrofuran (hereinafter, THF) (250 ml) was added and cooled to about −78° C. Under a nitrogen atmosphere, n-butyllithium (33 mmol) was slowly added dropwisely, followed by stirring at room temperature for about 1 hour. After washing with ethyl acetate and water three times, the organic layer thus obtained was dried over MgSO₄ and then, dried under a reduced pressure. Through column chromatography, Intermediate Compound 1-2 was obtained (yield 87%). The compound thus produced was identified through LC-MS (C₁₉H₂₀O₂:M+280.1).

### 3) Synthesis of Intermediate Compound 1-3

To Intermediate Compound 1-2 (20 ml), dichloromethane (hereinafter, DCM) (200 ml) was added, and the resultant was cooled to about 0° C. Under a nitrogen atmosphere, boron tribromide (BBr₃, 30 mmol) was slowly added dropwisely, followed by stirring at room temperature for about 1 hour. Triphenyl amine was slowly added dropwisely to neutralize, and the resultant was extracted with ethyl acetate and water three times. The organic layer thus obtained was dried over MgSO₄ and then, dried under a reduced pressure. Through column chromatography, Intermediate Compound 1-3 was obtained (yield 98%). The compound thus produced was identified through LC-MS (C₁₇H₁₈O₂:M+252.1).

### 4) Synthesis of Intermediate Compound 1-4

To Intermediate Compound 1-3 (10 ml), triphenylamine (20 mmol) and DCM (200 ml) were added, and the resultant was cooled to about 0° C. Under a nitrogen atmosphere, trifluoromethanesulfonic anhydride (20 mmol) was slowly added dropwisely, followed by stirring at room temperature for about 1 hour. The resultant was extracted with ethyl acetate and water three times. The organic layer thus obtained was dried over MgSO₄ and then, dried under a reduced pressure. Through column chromatography, Intermediate Compound 1-4 was obtained (yield 98%).

### 5) Synthesis of Intermediate Compound 1-5

Intermediate Compound 1-4 (1.0 eq.), aniline (2.2 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 1-5 was obtained (yield 87%).

### 6) Synthesis of Intermediate Compound 1-6

Intermediate Compound 1-5 (1.0 eq.), bromobenzene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 80° C. for about 1 hour. After cooling, the resultant was washed with ethyl acetate and water three times. The organic layer thus obtained was dried over MgSO₄ and then, dried under a reduced pressure. Through column chromatography, Intermediate Compound 1-6 was obtained (yield 85%).

### 7) Synthesis of Compound 1

Intermediate Compound 1-6 (1.0 eq.), 2-bromo-9,9-dimethyl-9H-fluorene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 1 was obtained (yield 80%). By FAB-MS measurement, mass number of m/z=670.33 was observed as a molecular ion peak, and Compound 1 was identified.

### (2) Synthesis of Compound 2

Amine Compound 2 according to one or more embodiments may be synthesized by, for example, the steps of Reaction 2 below.

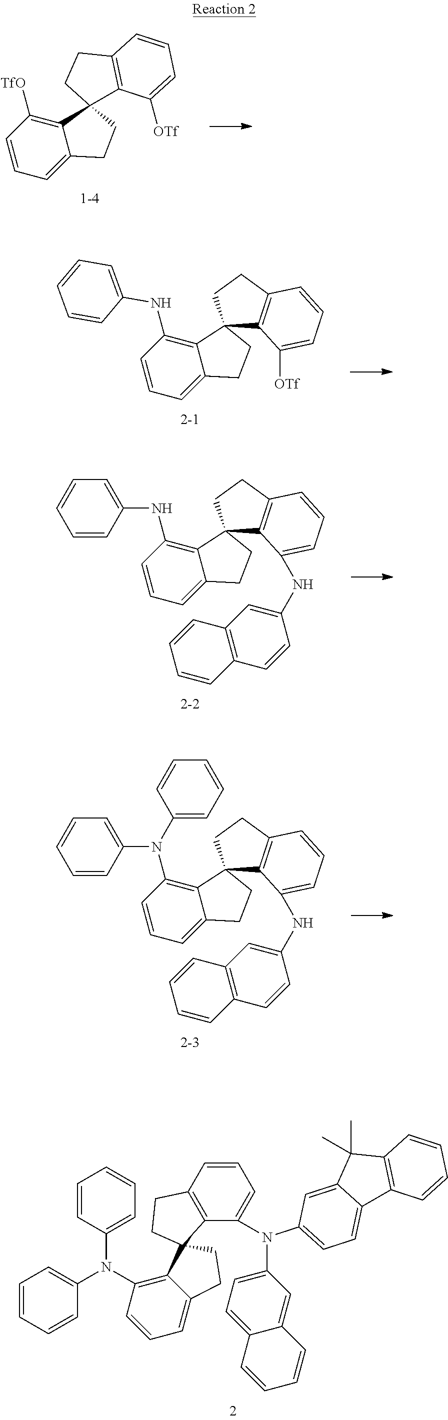

### 1) Synthesis of Intermediate Compound 2-1

Intermediate Compound 1-4 (1.0 eq.), aniline (1.0 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 2-1 was obtained (yield 45%).

### 2) Synthesis of Intermediate Compound 2-2

Intermediate Compound 2-1 (1.0 eq.), 2-aminonaphthalene (1.2 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 2-2 was obtained (yield 87%).

### 3) Synthesis of Intermediate Compound 2-3

Intermediate Compound 2-2 (1.0 eq.), bromobenzene (1.0 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 80° C. for about 1 hour. After cooling, the resultant was washed with ethyl acetate and water three times. The organic layer thus obtained was dried over MgSO₄ and then, dried under a reduced pressure. Through column chromatography, Intermediate Compound 2-3 was obtained (yield 46%).

### 4) Synthesis of Compound 2

Intermediate Compound 2-3 (1.0 eq.), 2-bromo-9,9-dimethyl-9H-fluorene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 2 was obtained (yield 80%). By FAB-MS measurement, mass number of m/z=720.35 was observed as a molecular ion peak, and Compound 2 was identified.

### (3) Synthesis of Compound 6

Amine Compound 6 according to one or more embodiments may be synthesized by, for example, the steps of Reaction 3 below.

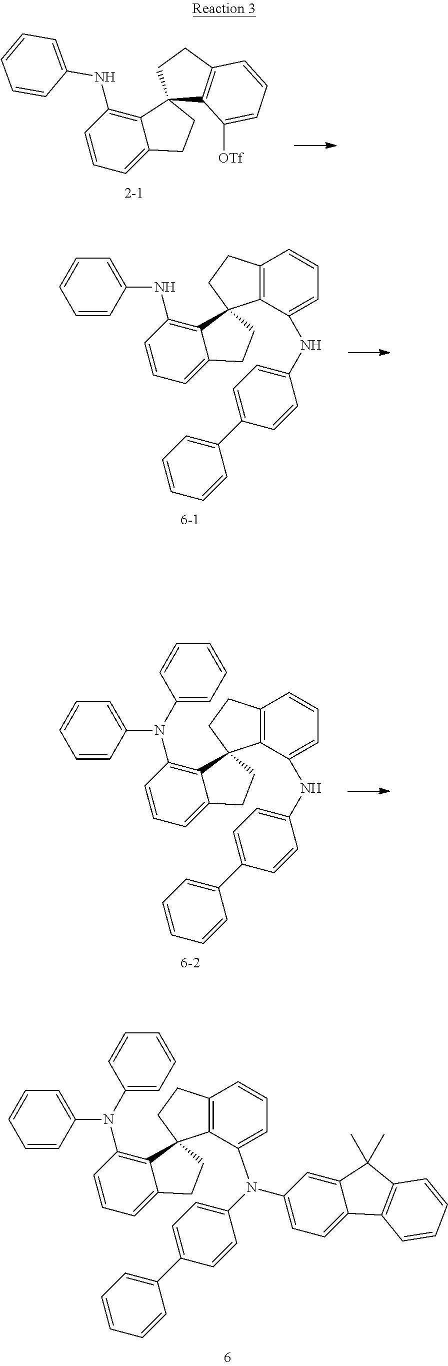 [SMILES: O=S(=O)(Oc1cccc2c1[C@]1(CCc3cccc(Nc4ccccc4)c31)CC2)C(F)(F)F ; c1ccc(Nc2cccc3c2[C@@]2(CC3)CCc3cccc(Nc4ccc(-c5ccccc5)cc4)c32)cc1 ; c1ccc(-c2ccc(Nc3cccc4c3[C@@]3(CC4)CCc4cccc(N(c5ccccc5)c5ccccc5)c43)cc2)cc1 ; CC1(C)c2ccccc2-c2ccc(N(c3ccc(-c4ccccc4)cc3)c3cccc4c3[C@]3(CCc5cccc(N(c6ccccc6)c6ccccc6)c53)CC4)cc21]

### 1) Synthesis of Intermediate Compound 6-1

Intermediate Compound 2-1 (1.0 eq.), [1,1′-biphenyl]-4-amine (1.0 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 6-1 was obtained (yield 75%).

### 2) Synthesis of Intermediate Compound 6-2

Intermediate Compound 6-1 (1.0 eq.), bromobenzene (1.0 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 80° C. for about 1 hour. After cooling, the resultant was washed with ethyl acetate and water three times. The organic layer thus obtained was dried over MgSO₄ and then, dried under a reduced pressure. Through column chromatography, Intermediate Compound 6-2 was obtained (yield 82%).

### 3) Synthesis of Compound 6

Intermediate Compound 6-2 (1.0 eq.), 2-bromo-9,9-dimethyl-9H-fluorene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 6 was obtained (yield 80%). By FAB-MS measurement, mass number of m/z=746.37 was observed as a molecular ion peak, and Compound 6 was identified.

### (4) Synthesis of Compound 14

Amine Compound 14 according to one or more embodiments may be synthesized by, for example, the steps of Reaction 4 below.

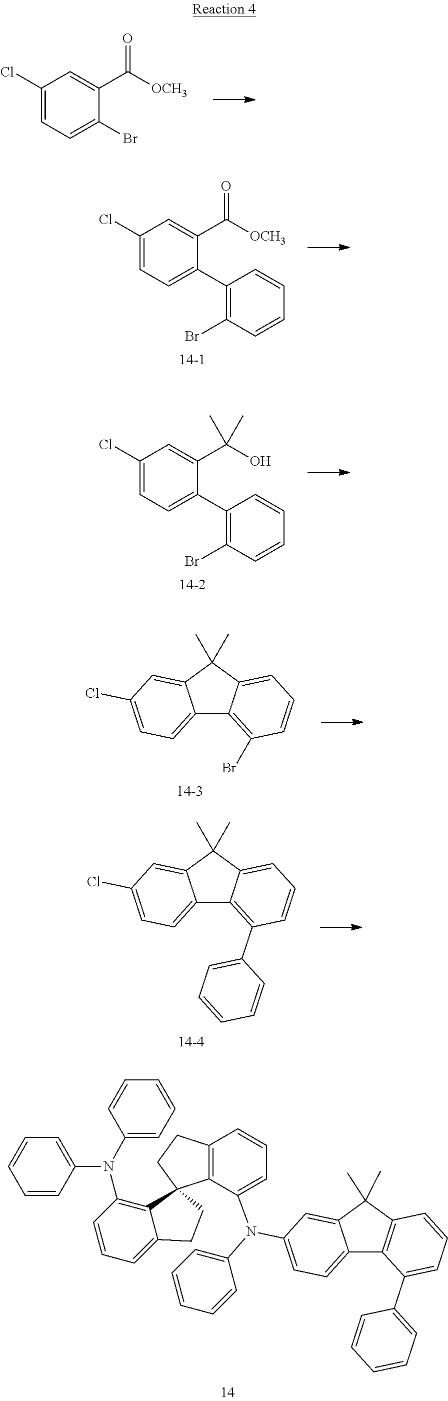 [SMILES: COC(=O)c1cc(Cl)ccc1Br ; COC(=O)c1cc(Cl)ccc1-c1ccccc1Br ; CC(C)(O)c1cc(Cl)ccc1-c1ccccc1Br ; CC1(C)c2cc(Cl)ccc2-c2c(Br)cccc21 ; CC1(C)c2cc(Cl)ccc2-c2c(-c3ccccc3)cccc21 ; CC1(C)c2cc(N(c3ccccc3)c3cccc4c3[C@]3(CCc5cccc(N(c6ccccc6)c6ccccc6)c53)CC4)ccc2-c2c(-c3ccccc3)cccc21]

### 1) Synthesis of Intermediate Compound 14-1

To methyl 2-bromo-5-chlorobenzoate (1.0 eq.), 2-bromophenylboronic acid (1.2 eq.), tetrakis(triphenylphosphine)palladium (0.05 eq.), and potassium carbonate (2.0 eq.) were dissolved in THF:H₂O in a volume ratio of about 4:1, and stirred under a nitrogen atmosphere at about 80° C. for about 12 hours. After cooling, the reaction solution was washed with ethyl acetate and water three times. The organic layer thus obtained was dried over MgSO₄, and then, dried under a reduced pressure. Through column chromatography, Intermediate Compound 14-1 was obtained (yield 85%).

### 2) Synthesis of Intermediate Compound 14-2

To Intermediate Compound 14-1 (1.0 eq.), THF (200 ml) was added and cooled to about 0° C. Under a nitrogen atmosphere, methylmagnesium bromide (4.0 eq.) was slowly added dropwisely, followed by stirring for about 1 hour. An aqueous ammonium chloride solution was slowly added dropwisely to neutralize, and the resultant was extracted with ethyl acetate and water three times. The organic layer thus obtained was dried over MgSO₄ and then, dried under a reduced pressure. Through column chromatography, Intermediate Compound 14-2 was obtained (yield 78%).

### 3) Synthesis of Intermediate Compound 14-3

To Intermediate Compound 14-2 (1.0 eq.), and DCM (200 ml), trifluoromethanesulfonic acid (10 eq.) was slowly added dropwisely, followed by stirring at about 50° C. After cooling to room temperature, the resultant was extracted with ethyl acetate and water three times. The organic layer thus obtained was dried over MgSO₄ and then, dried under a reduced pressure. Through column chromatography, Intermediate Compound 14-3 was obtained (yield 72%).

### 4) Synthesis of Intermediate Compound 14-4

Intermediate Compound 14-3 (1.0 eq.), phenylboronic acid (1.2 eq.), tetrakis(triphenylphosphine)palladium (0.05 eq.), and potassium carbonate (2.0 eq.) were dissolved in THF:H₂O in a volume ratio of about 4:1, followed by stirring under a nitrogen atmosphere at about 80° C. for about 12 hours. After cooling, the resultant was washed with ethyl acetate and water three times. The organic layer thus obtained was dried over MgSO₄ and then, dried under a reduced pressure. Through column chromatography, Intermediate Compound 14-4 was obtained (yield 85%).

### 5) Synthesis of Compound 14

Intermediate Compound 1-6 (1.0 eq.), Intermediate Compound 14-4 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 14 was obtained (yield 80%). By FAB-MS measurement, mass number of m/z=746.37 was observed as a molecular ion peak, and Compound 14 was identified.

### (5) Synthesis of Compound 16

Amine Compound 16 according to one or more embodiments may be synthesized by, for example, the steps of Reaction 5 below.

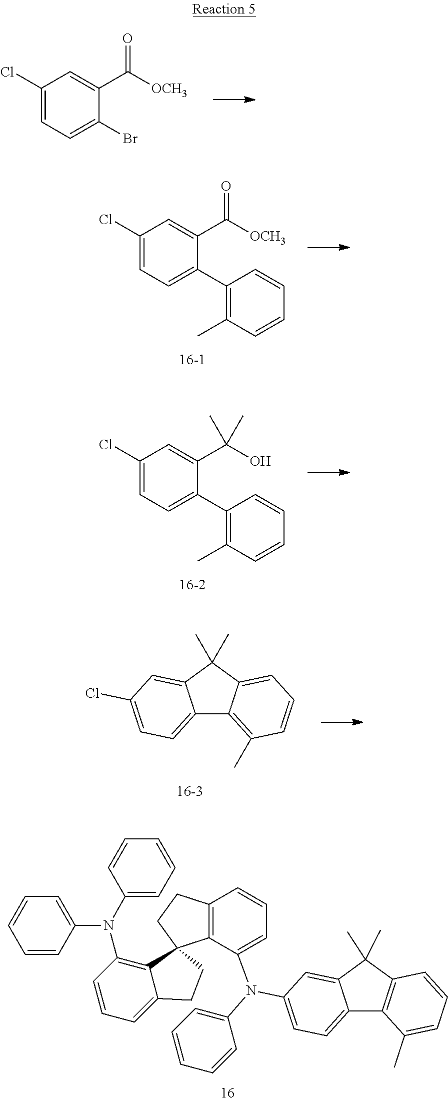 [SMILES: COC(=O)c1cc(Cl)ccc1Br ; COC(=O)c1cc(Cl)ccc1-c1ccccc1C ; Cc1ccccc1-c1ccc(Cl)cc1C(C)(C)O ; Cc1cccc2c1-c1ccc(Cl)cc1C2(C)C ; Cc1cccc2c1-c1ccc(N(c3ccccc3)c3cccc4c3[C@]3(CCc5cccc(N(c6ccccc6)c6ccccc6)c53)CC4)cc1C2(C)C]

### 1) Synthesis of Intermediate Compound 16-1

To methyl 2-bromo-5-chlorobenzoate (1.0 eq.), 2-methylphenylboronic acid (1.2 eq.), tetrakis(triphenylphosphine)palladium (0.05 eq.), and potassium carbonate (2.0 eq.) were dissolved in THF:H₂O in a volume ratio of about 4:1, and stirred under a nitrogen atmosphere at about 80° C. for about 12 hours. After cooling, the resultant was washed with ethyl acetate and water three times. The organic layer thus obtained was dried over MgSO₄, and then, dried under a reduced pressure. Through column chromatography, Intermediate Compound 16-1 was obtained (yield 85%).

### 2) Synthesis of Intermediate Compound 16-2

To Intermediate Compound 16-1 (1.0 eq.), THF (200 ml) was added and cooled to about 0° C. Under a nitrogen atmosphere, methylmagnesium bromide (4.0 eq.) was slowly added dropwisely, followed by stirring for about 1 hour. An aqueous ammonium chloride solution was slowly added dropwisely to neutralize, and the resultant was extracted with ethyl acetate and water three times. The organic layer thus obtained was dried over MgSO₄ and then, dried under a reduced pressure. Through column chromatography, Intermediate Compound 16-2 was obtained (yield 78%).

### 3) Synthesis of Intermediate Compound 16-3

To Intermediate Compound 16-2 (1.0 eq.) and DCM (200 ml), trifluoromethanesulfonic acid (10 eq.) was slowly added dropwisely, followed by stirring at about 50° C. After cooling to room temperature, the resultant was extracted with ethyl acetate and water three times. The organic layer thus obtained was dried over MgSO₄ and then, dried under a reduced pressure. Through column chromatography, Intermediate Compound 16-3 was obtained (yield 72%).

### 4) Synthesis of Compound 16

Intermediate Compound 1-6 (1.0 eq.), Intermediate Compound 16-3 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 16 was obtained (yield 80%). By FAB-MS measurement, mass number of m/z=684.35 was observed as a molecular ion peak, and Compound 16 was identified.

### (6) Synthesis of Compound 17

Amine Compound 17 according to one or more embodiments may be synthesized by, for example, the steps of Reaction 6 below.

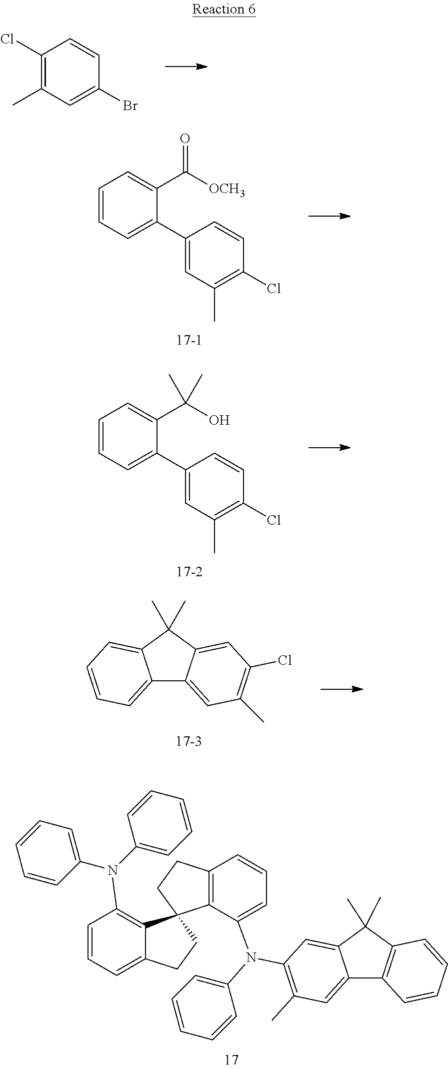

### 1) Synthesis of Intermediate Compound 17-1

2-(2-(Methoxycarbonyl)phenyl)boronic acid (1.0 eq.), 4-bromo-1-chloro-2-methylbenzene (1.2 eq.), tetrakis(triphenylphosphine)palladium (0.05 eq.), and potassium carbonate (2.0 eq.) were dissolved in THF:H₂O in a volume ratio of about 4:1, and stirred under a nitrogen atmosphere at about 80° C. for about 12 hours. After cooling, the resultant was washed with ethyl acetate and water three times. The organic layer thus obtained was dried over MgSO₄, and then, dried under a reduced pressure. Through column chromatography, Intermediate Compound 17-1 was obtained (yield 85%).

### 2) Synthesis of Intermediate Compound 17-2

To Intermediate Compound 17-1 (1.0 eq.), THF (200 ml) was added and cooled to about 0° C. Under a nitrogen atmosphere, methylmagnesium bromide (4.0 eq.) was slowly added dropwisely, followed by stirring for about 1 hour. An aqueous ammonium chloride solution was slowly added dropwisely to neutralize, and the resultant was extracted with ethyl acetate and water three times. The organic layer thus obtained was dried over MgSO₄ and then, dried under a reduced pressure. Through column chromatography, Intermediate Compound 17-2 was obtained (yield 78%).

### 3) Synthesis of Intermediate Compound 17-3

To Intermediate Compound 17-2 (1.0 eq.) and DCM (200 ml), trifluoromethanesulfonic acid (10 eq.) was slowly added dropwisely, followed by stirring at about 50° C. After cooling to room temperature, the resultant was extracted with ethyl acetate and water three times. The organic layer thus obtained was dried over MgSO₄ and then, dried under a reduced pressure. Through column chromatography, Intermediate Compound 17-3 was obtained (yield 72%).

### 4) Synthesis of Compound 17

Intermediate Compound 17-3 (1.0 eq.), Intermediate Compound 14-4 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 17 was obtained (yield 80%). By FAB-MS measurement, mass number of m/z=684.35 was observed as a molecular ion peak, and Compound 17 was identified.

### (7) Synthesis of Compound 24

Amine Compound 24 according to one or more embodiments may be synthesized by, for example, the steps of Reaction 7 below.

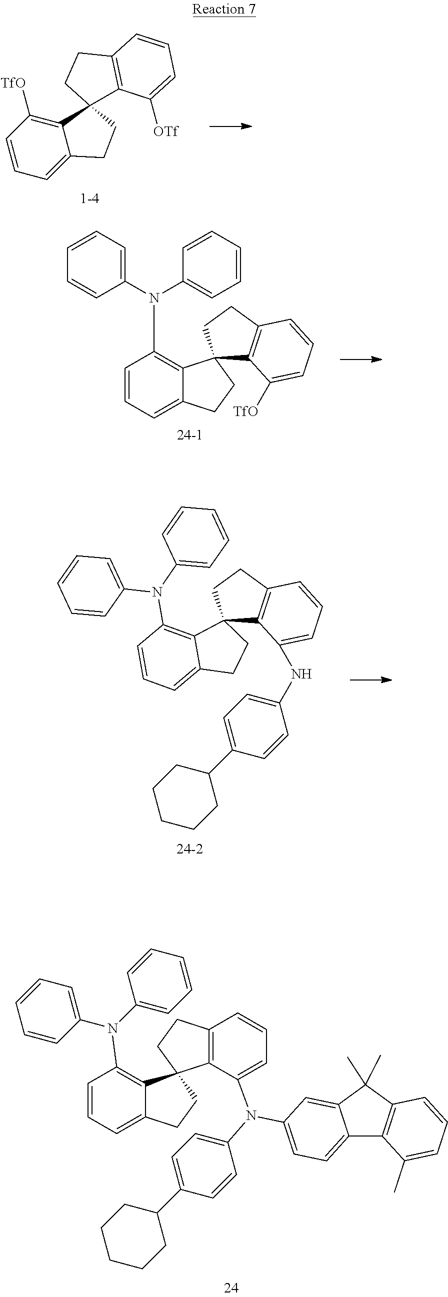

### 1) Synthesis of Intermediate Compound 24-1

Intermediate Compound 1-4 (1.0 eq.), diphenylamine (1.0 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 24-1 was obtained (yield 75%).

### 2) Synthesis of Intermediate Compound 24-2

Intermediate Compound 24-1 (1.0 eq.), 4-cyclohexylaniline (1.0 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Intermediate Compound 24-2 was obtained (yield 75%).

### 3) Synthesis of Compound 24

Intermediate Compound 24-2 (1.0 eq.), Intermediate Compound 16-3 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 24 was obtained (yield 80%). By FAB-MS measurement, mass number of m/z=766.43 was observed as a molecular ion peak, and Compound 24 was identified.

### (8) Synthesis of Compound 402

Amine Compound 402 according to one or more embodiments may be synthesized by, for example, the step of Reaction 8 below.

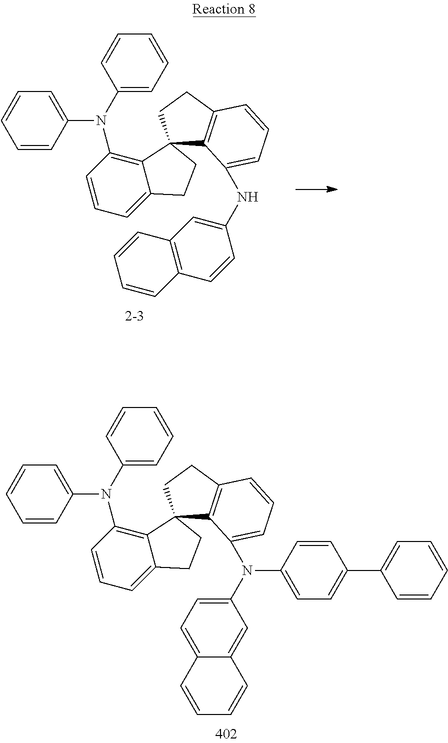 [SMILES: c1ccc(N(c2ccccc2)c2cccc3c2[C@]2(CCc4cccc(Nc5ccc6ccccc6c5)c42)CC3)cc1 ; c1ccc(-c2ccc(N(c3ccc4ccccc4c3)c3cccc4c3[C@]3(CCc5cccc(N(c6ccccc6)c6ccccc6)c53)CC4)cc2)cc1]

### 1) Synthesis of Compound 402

Intermediate Compound 2-3 (1.0 eq.), 4-bromo-1,1′-biphenyl (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 402 was obtained (yield 80%). By FAB-MS measurement, mass number of m/z=680.32 was observed as a molecular ion peak, and Compound 402 was identified.

### (9) Synthesis of Compound 422

Amine Compound 422 according to one or more embodiments may be synthesized by, for example, the step of Reaction 9 below.

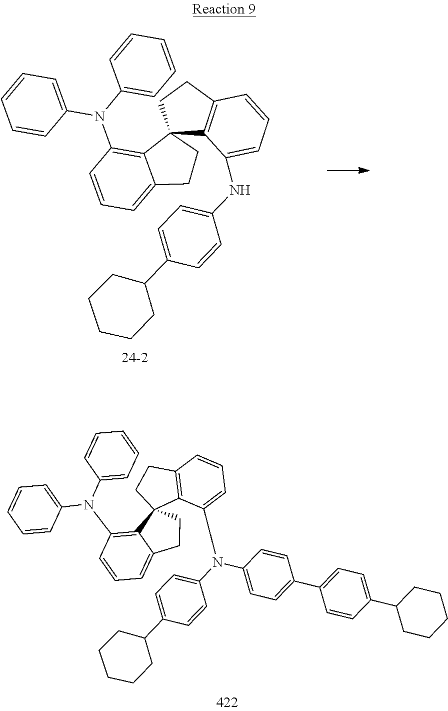 [SMILES: c1ccc(N(c2ccccc2)c2cccc3c2[C@]2(CCc4cccc(Nc5ccc(C6CCCCC6)cc5)c42)CC3)cc1 ; c1ccc(N(c2ccccc2)c2cccc3c2[C@@]2(CC3)CCc3cccc(N(c4ccc(-c5ccc(C6CCCCC6)cc5)cc4)c4ccc(C5CCCCC5)cc4)c32)cc1]

### 1) Synthesis of Compound 422

Intermediate Compound 24-2 (1.0 eq.), 4-bromo-4′-cyclohexyl-1,1′-biphenyl (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 422 was obtained (yield 80%). By FAB-MS measurement, mass number of m/z=794.46 was observed as a molecular ion peak, and Compound 422 was identified.

### (10) Synthesis of Compound 451

Amine Compound 451 according to one or more embodiments may be synthesized by, for example, the steps of Reaction 10 below.

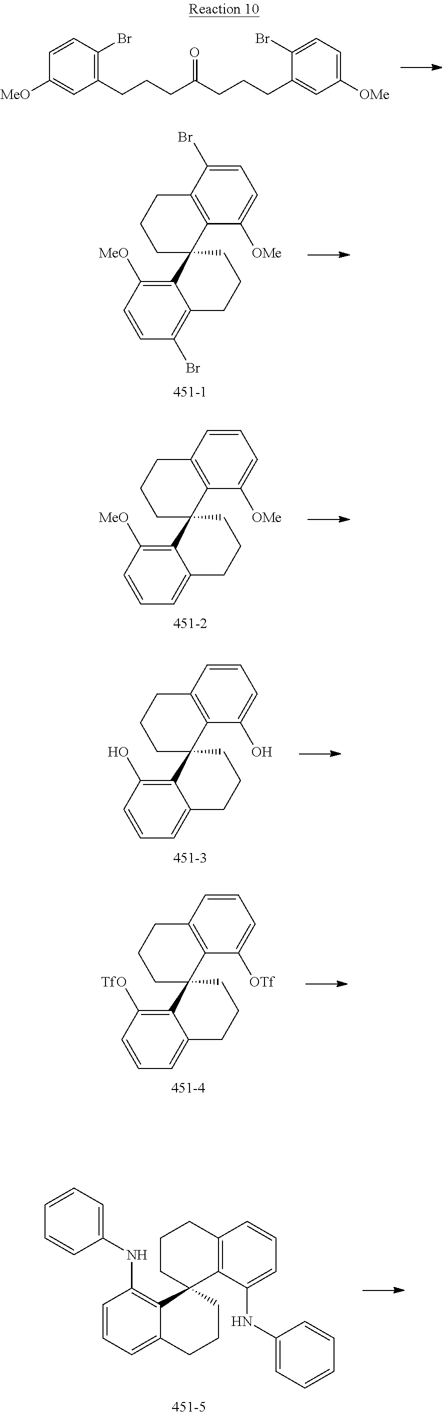

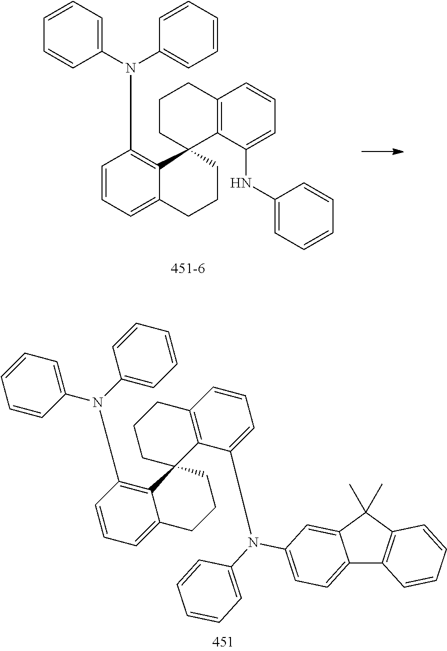 [SMILES: c1ccc(Nc2cccc3c2[C@@]2(CCC3)CCCc3cccc(N(c4ccccc4)c4ccccc4)c32)cc1 ; CC1(C)c2ccccc2-c2ccc(N(c3ccccc3)c3cccc4c3[C@]3(CCCc5cccc(N(c6ccccc6)c6ccccc6)c53)CCC4)cc21]

### 1) Synthesis of Intermediate Compound 451-1

To 1,7-bis(2-bromo-5-methoxyphenyl)heptan-4-one (50 mmol), polyphosphoric acid (150 g) was added and stirred at about 105° C. for about 6 hours. Then, the reaction solution was cooled to room temperature, and the reaction solution was added to 300 mol of water, followed by extracting with dichloromethane three times. The organic layer thus obtained was dried over MgSO₄, and the residue obtained by vaporizing the solvent was separated by silica gel chromatography to obtain Intermediate Compound 451-1 (yield 67%).

### 2) Synthesis of Intermediate Compound 451-2

To Intermediate Compound 451-1 (30 ml), THF (250 ml) was added and cooled to about −78° C. Under a nitrogen atmosphere, n-butyllithium (33 mmol) was slowly added dropwisely, followed by stirring at room temperature for about 1 hour. The resultant was washed with ethyl acetate and water three times. The organic layer thus obtained was dried over MgSO₄ and then, dried under a reduced pressure. Through column chromatography, Intermediate Compound 451-2 was obtained (yield 87%).

### 3) Synthesis of Intermediate Compound 451-3

To Intermediate Compound 451-2 (20 ml), DCM (200 ml) was added, followed by cooling to about 0° C. Under a nitrogen atmosphere, boron tribromide (BBr₃, 30 mmol) was slowly added dropwisely, followed by stirring at room temperature for about 1 hour. Triphenylamine was slowly added dropwisely to neutralize, and an organic layer obtained by extracting the resultant with ethyl acetate and water three times was dried over MgSO₄ and then, dried under a reduced pressure. Through column chromatography, Intermediate Compound 451-3 was obtained (yield 98%).

### 4) Synthesis of Intermediate Compound 451-4

To Intermediate Compound 451-3, triphenylamine (20 mmol) and DCM (200 mL) were added and cooled to about 0° C. Under a nitrogen atmosphere, trifluoromethanesulfonic anhydride (20 mmol) was slowly added dropwisely, followed by stirring at room temperature for about 1 hour. An organic layer obtained by extracting the resultant with ethyl acetate and water three times was dried over MgSO₄ and then, dried under a reduced pressure. Through column chromatography, Intermediate Compound 451-4 was obtained (yield 98%).

### 5) Synthesis of Intermediate Compound 451-5

Intermediate Compound 451-4 (1.0 eq.), aniline (2.2 eq.), tris(dibenzylideneacetone)dipalladium(0) (0.05 eq.), 2-dicyclohexylphosphino-2′,6′-dimethoxybiphenyl (Sphos, 0.10 eq.), and sodium tert-butoxide (2.0 eq.) were dissolved in toluene, followed by stirring under a nitrogen atmosphere at about 100° C. for about 1 hours. After cooling, the resultant was washed with ethyl acetate and water three times, and the organic layer thus obtained was dried over MgSO₄, and then, dried under a reduced pressure. Through column chromatography, Intermediate Compound 451-5 was obtained (yield 87%).

### 6) Synthesis of Intermediate Compound 451-6

Intermediate Compound 451-5 (1.0 eq.), bromobenzene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 80° C. for about 1 hour. After cooling, the resultant was washed with ethyl acetate and water three times. The organic layer thus obtained was dried over MgSO₄ and then, dried under a reduced pressure. Through column chromatography, Intermediate Compound 451-6 was obtained (yield 85%).

### 7) Synthesis of Compound 451

Intermediate Compound 451-6 (1.0 eq.), 2-bromo-9,9-dimethyl-9H-fluorene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under a nitrogen atmosphere at about 100° C. for about 1 hour. After cooling, the resultant was washed with ethyl acetate and water three times. The organic layer thus obtained was dried over MgSO₄ and then, dried under a reduced pressure. Through column chromatography, Compound 451 was obtained (yield 80%). By FAB-MS measurement, mass number of m/z=698.37 was observed as a molecular ion peak, and Compound 451 was identified.

### 2. Manufacture and Evaluation of Light Emitting Elements

Evaluation of light emitting elements including the compounds of the Example and the Comparative Examples in hole transport layers was conducted by a method below. The manufacturing methods for evaluating the light emitting elements are described below.

### (1) Manufacture of Light Emitting Elements 1

The light emitting elements of embodiments, including the amine compounds of embodiments in hole transport layers were manufactured by a method below. Light emitting elements of Example 1 to Example 5 were manufactured using Compounds 1, 2, 6, 14, and 402 as hole transport materials. The light emitting element of Comparative Example 1 was manufactured using a known material of NPB as a hole transport material. The light emitting element of Comparative Example 2 was manufactured using Compound H-1-20 as a hole transport material. Comparative Examples 3 and 4 were manufactured using Compound A and Compound B as hole transport materials, respectively. Comparative Examples 5 and 6 were manufactured using Compound C and Compound D as hole transport materials, respectively.

For the manufacture of each of the light emitting elements of Example 1 to Example 5, and Comparative Example 1 to Comparative Example 6, an ITO glass substrate with about 15 Ω/cm² (1200 Å) of Corning Co. was cut into a size of 50 mm×50 mm×0.7 mm, washed by ultrasonic waves using isopropyl alcohol and distilled water for about 5 minutes each, and cleansed by exposing to ultraviolet rays for about 30 minutes and exposing to ozone, and this glass substrate was installed in a vacuum deposition apparatus.

On the ITO glass substrate, 2-TNATA was vacuum deposited to a thickness of about 600 Å to form a hole injection layer, and the Example Compound or Comparative Compound was vacuum deposited to a thickness of about 300 Å to form a hole transport layer.

Then, on the hole transport layer, 9,10-di(naphthlen-2-yl)anthracene (hereinafter, ADN) as a blue fluorescence host and 4,4′-bis[2-(4-(N,N-diphenylamino)phenyl)vinyl]biphenyl (hereinafter, DPAVBi) as a blue fluorescence dopant were deposited at the same time in a weight ratio of about 98:2 to a thickness of about 300 Å to form an emission layer.

Then, on the emission layer, Alq₃ was deposited to a thickness of about 300 Å to form an electron transport layer, and on the electron transport layer, an alkali metal halide of LiF was deposited to a thickness of about 10 Å to form an electron injection layer. On the electron injection layer, Al was vacuum deposited to a thickness of about 3000 Å to form a second electrode.

### (2) Manufacture of Light Emitting Elements 2

The light emitting elements of embodiments, including the amine compounds of embodiments in hole transport layers were manufactured by a method below. Light emitting elements of Example 6 to Example 11 were manufactured using Compounds 15, 16, 17, 24, 422 and 451, and Compound H-1-20 as hole transport materials. The light emitting element of Comparative Example 7 was manufactured using Compound A, and Compound H-1-20 as hole transport materials. For the manufacture of each of the light emitting elements of Example 6 to Example 11, and Comparative Example 7, an ITO glass substrate with about 15 Ω/cm² (1200 Å) of Corning Co. was cut into a size of 50 mm×50 mm×0.7 mm, washed by ultrasonic waves using isopropyl alcohol and distilled water for about 5 minutes each, and cleansed by exposing to ultraviolet rays for about 30 minutes and exposing to ozone, and this glass substrate was installed in a vacuum deposition apparatus.

On the ITO glass substrate, 2-TNATA was vacuum deposited to a thickness of about 600 Å to form a hole injection layer, and the Example Compound or Comparative Compound was vacuum deposited to a thickness of about 100 Å to form a first hole transport layer. Then, on the first hole transport layer, Compound H-1-20 was vacuum deposited to a thickness of about 100 Å to form a second hole transport layer, and on the second hole transport layer, the Example Compound or Comparative Compound was vacuum deposited to a thickness of about 100 Å to form a third hole transport layer.

For example, Example 6 to Example 11 used Amine Compounds 15, 16, 17, 24, 422 and 451 of embodiments, respectively, for forming the first and third hole transport layers, and used Compound H-1-20 for forming the second hole transport layer. Comparative Example 7 used Compound A for forming the first and third hole transport layers and used Compound H-1-20 for forming the second hole transport layer.

Then, on the third hole transport layer, 9,10-di(naphthlen-2-yl)anthracene (hereinafter, ADN) as a blue fluorescence host and 4,4′-bis[2-(4-(N,N-diphenylamino)phenyl)vinyl]biphenyl (hereinafter, DPAVBi) as a blue fluorescence dopant were deposited at the same time in a weight ratio of about 98:2 to a thickness of about 300 Å to form an emission layer.

Then, on the emission layer, Alq₃ was deposited to a thickness of about 300 Å to form an electron transport layer, and on the electron transport layer, an alkali metal halide of LiF was deposited to a thickness of about 10 Å to form an electron injection layer. On the electron injection layer, Al was vacuum deposited to a thickness of about 3000 Å to form a second electrode. The compounds used for the manufacture of the light emitting elements of the Examples and Comparative Examples are shown below. The materials below were used for the manufacture of the light emitting elements after purchasing commercially available materials and performing sublimation purification.

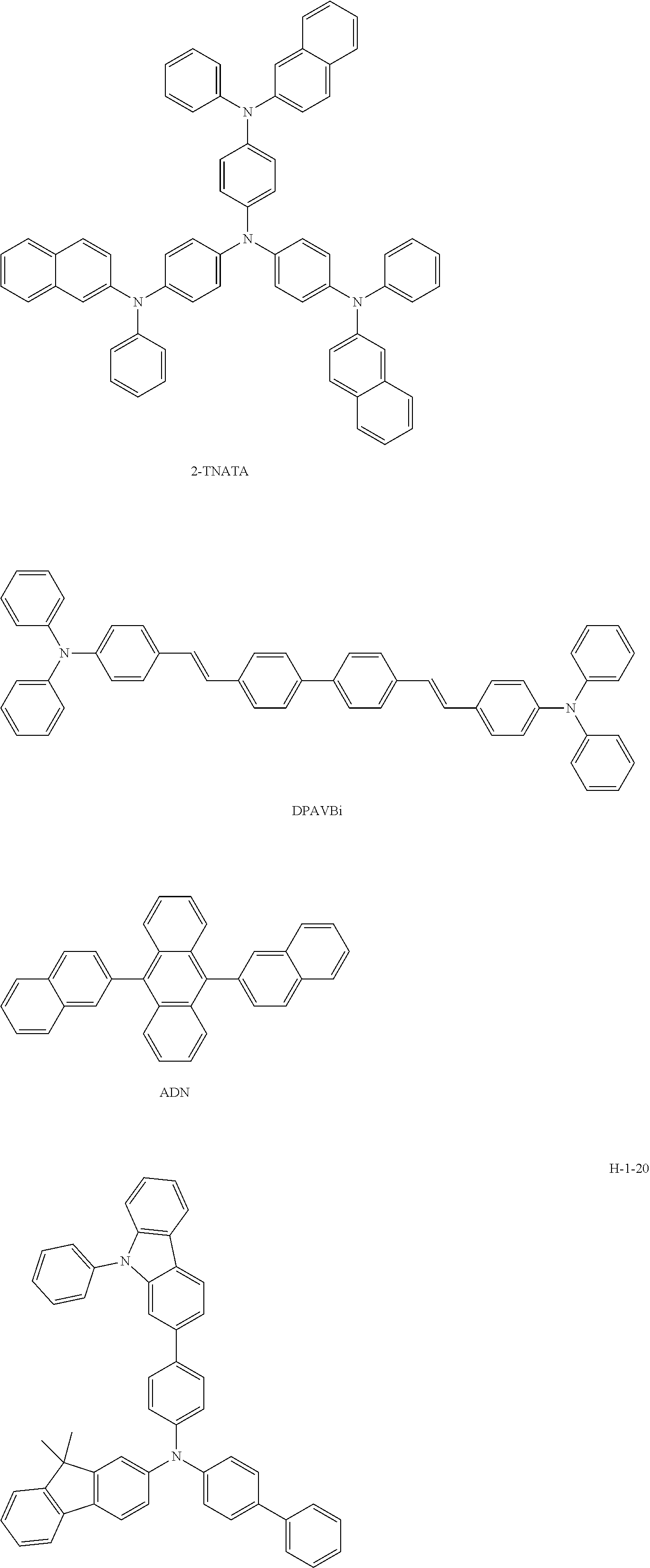

The Example Compounds and Comparative Compounds used for the manufacture of light emitting elements 1 and light emitting elements 2 are shown below.

### Example Compounds

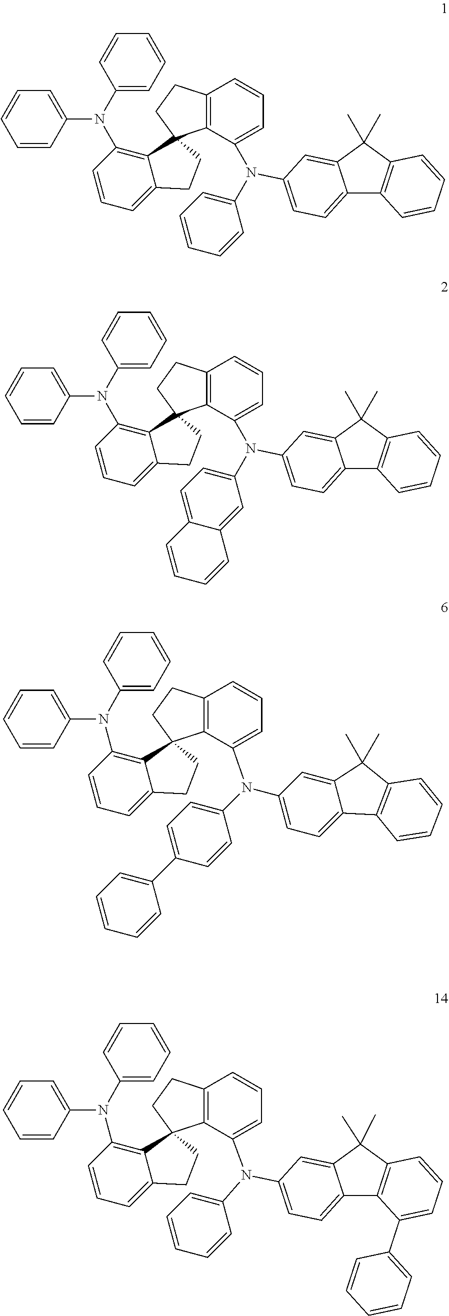

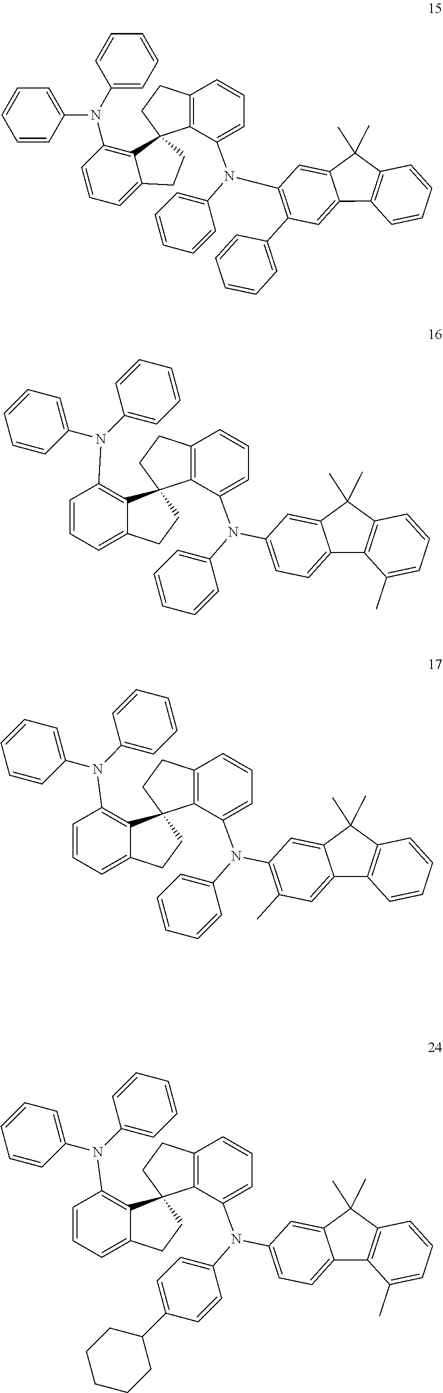 [SMILES: CC1(C)c2ccccc2-c2cc(-c3ccccc3)c(N(c3ccccc3)c3cccc4c3[C@]3(CCc5cccc(N(c6ccccc6)c6ccccc6)c53)CC4)cc21 ; Cc1cccc2c1-c1ccc(N(c3ccccc3)c3cccc4c3[C@]3(CCc5cccc(N(c6ccccc6)c6ccccc6)c53)CC4)cc1C2(C)C ; Cc1cc2c(cc1N(c1ccccc1)c1cccc3c1[C@]1(CCc4cccc(N(c5ccccc5)c5ccccc5)c41)CC3)C(C)(C)c1ccccc1-2 ; Cc1cccc2c1-c1ccc(N(c3ccc(C4CCCCC4)cc3)c3cccc4c3[C@]3(CCc5cccc(N(c6ccccc6)c6ccccc6)c53)CC4)cc1C2(C)C]

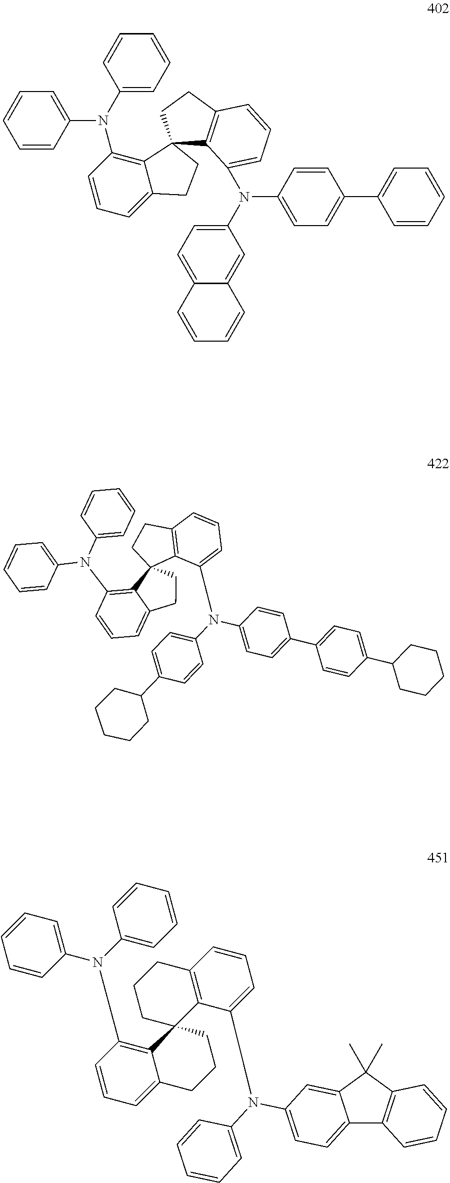 [SMILES: c1ccc(-c2ccc(N(c3ccc4ccccc4c3)c3cccc4c3[C@]3(CCc5cccc(N(c6ccccc6)c6ccccc6)c53)CC4)cc2)cc1 ; c1ccc(N(c2ccccc2)c2cccc3c2[C@@]2(CC3)CCc3cccc(N(c4ccc(-c5ccc(C6CCCCC6)cc5)cc4)c4ccc(C5CCCCC5)cc4)c32)cc1 ; CC1(C)c2ccccc2-c2ccc(N(c3ccccc3)c3cccc4c3[C@]3(CCCc5cccc(N(c6ccccc6)c6ccccc6)c53)CCC4)cc21]

### Comparative Compounds

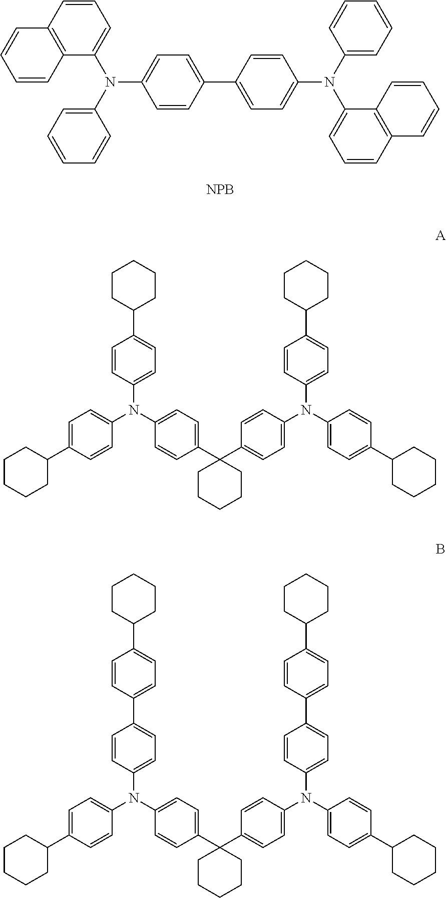 [SMILES: c1ccc(N(c2ccc(-c3ccc(N(c4ccccc4)c4cccc5ccccc45)cc3)cc2)c2cccc3ccccc23)cc1 ; "NPB" ; c1cc(N(c2ccc(C3CCCCC3)cc2)c2ccc(C3(c4ccc(N(c5ccc(C6CCCCC6)cc5)c5ccc(C6CCCCC6)cc5)cc4)CCCCC3)cc2)ccc1C1CCCCC1 ; c1cc(C2CCCCC2)ccc1-c1ccc(N(c2ccc(C3CCCCC3)cc2)c2ccc(C3(c4ccc(N(c5ccc(-c6ccc(C7CCCCC7)cc6)cc5)c5ccc(C6CCCCC6)cc5)cc4)CCCCC3)cc2)cc1]

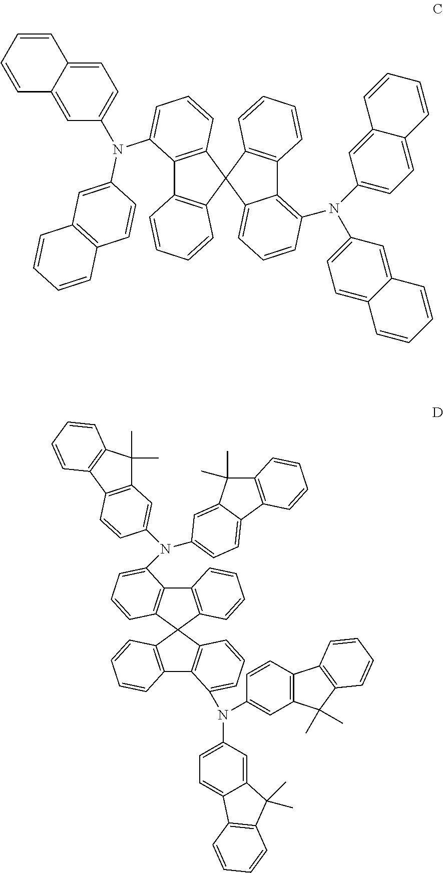 [SMILES: c1ccc2c(c1)-c1c(N(c3ccc4ccccc4c3)c3ccc4ccccc4c3)cccc1C21c2ccccc2-c2c(N(c3ccc4ccccc4c3)c3ccc4ccccc4c3)cccc21 ; CC1(C)c2ccccc2-c2ccc(N(c3ccc4c(c3)C(C)(C)c3ccccc3-4)c3cccc4c3-c3ccccc3C43c4ccccc4-c4c(N(c5ccc6c(c5)C(C)(C)c5ccccc5-6)c5ccc6c(c5)C(C)(C)c5ccccc5-6)cccc43)cc21]

### (3) Evaluation of Light Emitting Elements

The driving voltages, luminance, emission efficiency and half life of the manufactured light emitting elements were evaluated. In Table 1, the evaluation results of the light emitting elements of Example 1 to Example 11 and Comparative Examples 1 to 7 are shown. In the evaluation results of the properties for the Examples and Comparative Examples, shown in Table 1, the driving voltage and current density were measured using V7000 OLED IVL Test System (Polaronix). In order to evaluate the properties of the light emitting elements manufactured in Example 1 to Example 11 and Comparative Examples 1 to 7, a driving voltage at a current density of about 50 mA/cm² and efficiency (cd/A) were measured, and the half life was evaluated as the time from an initial value of luminance to 50% of luminance deterioration while driving continuously at a current density of about 100 mA/cm².

TABLE 1

| | Hole | Driving | Current | | | | Half life |
|---|---|---|---|---|---|---|---|
| | transport | voltage | density | Luminance | Efficiency | Emission | (hr @100 |
| | material | (V) | (mA/cm²) | (cd/m²) | (cd/A) | color | mA/cm²) |
| Example 1 | Compound 1 | 4.8 | 50 | 3600 | 7.2 | Blue | 350 |
| Example 2 | Compound 2 | 4.6 | 50 | 3550 | 7.1 | Blue | 340 |
| Example 3 | Compound 6 | 4.6 | 50 | 3650 | 7.3 | Blue | 365 |
| Example 4 | Compound 14 | 4.7 | 50 | 3550 | 7.1 | Blue | 380 |
| Example 5 | Compound 402 | 4.9 | 50 | 3450 | 6.9 | Blue | 330 |
| Example 6 | Compound 15/ | 4.9 | 50 | 3800 | 7.6 | Blue | 380 |
| | Compound | | | | | | |
| | H-1-20 | | | | | | |
| Example 7 | Compound 16/ | 4.7 | 50 | 3705 | 7.41 | Blue | 385 |
| | Compound | | | | | | |
| | H-1-20 | | | | | | |
| Example 8 | Compound | 4.8 | 50 | 3750 | 7.5 | Blue | 370 |
| | 17/Compound | | | | | | |
| | H-1-20 | | | | | | |
| Example 9 | Compound | 4.9 | 50 | 3820 | 7.64 | Blue | 375 |
| | 24/Compound | | | | | | |
| | H-1-20 | | | | | | |
| Example | Compound | 4.8 | 50 | 3650 | 7.3 | Blue | 369 |
| 10 | 422/Compound | | | | | | |
| | H-1-20 | | | | | | |
| Example | Compound | 5.1 | 50 | 3680 | 7.36 | Blue | 360 |
| 11 | 451/Compound | | | | | | |
| | H-1-20 | | | | | | |
| Com- | NPB | 7.0 | 50 | 2645 | 5.29 | Blue | 258 |
| parative | | | | | | | |
| Example | | | | | | | |
| 1 | | | | | | | |
| Com- | Compound | 5.2 | 50 | 3200 | 6.4 | Blue | 300 |
| parative | H-1-20 | | | | | | |
| Example | | | | | | | |
| 2 | | | | | | | |
| Com- | Compound | 6.3 | 50 | 2300 | 4.6 | Blue | 260 |
| parative | A | | | | | | |
| Example | | | | | | | |
| 3 | | | | | | | |
| Com- | Compound | 6.0 | 50 | 2320 | 4.64 | Blue | 270 |
| parative | B | | | | | | |
| Example | | | | | | | |
| 4 | | | | | | | |
| Com- | Compound | 5.2 | 50 | 2900 | 5.8 | Blue | 275 |
| parative | C | | | | | | |
| Example | | | | | | | |
| 5 | | | | | | | |
| Com- | Compound | 5.3 | 50 | 2750 | 5.5 | Blue | 305 |
| parative | D | | | | | | |
| Example | | | | | | | |
| 6 | | | | | | | |
| Com- | Compound | 4.9 | 50 | 3408 | 6.82 | Blue | 310 |
| parative | A/Compound | | | | | | |
| Example | H-1-20 | | | | | | |
| 7 | | | | | | | |

Referring to the results of Table 1, it could be confirmed that the Examples of the light emitting elements using the amine compounds according to embodiments of the present disclosure as the materials of hole transport layers emit the same blue light, and show low driving voltages, improved emission efficiency and lifetime characteristics when compared to the Comparative Examples.

The amine compound according to one or more embodiments may include a spiro-bonded linker of two hydrocarbon rings and two amine groups connected with the linker. In the amine compound of one or more embodiments, the linker is spiro-bonded two hydrocarbon rings each of which has a fused structure of one benzene ring and one cycloalkyl group. The amine compound of one or more embodiments includes the linker to reduce intermolecular interaction and have low packing density. The amine compound of one or more embodiments, having such a structure may have high glass transition temperature and high melting point due to a large molecular weight and improved rigidity of a center part, and accordingly, may show excellent or improved heat resistance and durability properties.

In some embodiments, by diversely (e.g., variously) changing the substitution positions of two amine groups connected to the linker, the amine compound of one or more embodiments may diversely change the highest occupied molecular orbital (HOMO) energy level of a molecule. Accordingly, if the amine compound of one or more embodiments is applied in a hole transport region, hole transport properties may be improved, and accordingly, the recombination probability of holes and electrons in an emission layer may increase to improve emission efficiency.

In comparison, it could be confirmed that Comparative Example 1 to Comparative Example 7 used compounds not including a spiro-bonded linker of two hydrocarbon rings each of which has a fused structure of one benzene ring and one cycloalkyl group, as the materials of hole transport layers, and showed low thermal stability and reduced hole transport properties including deteriorated emission efficiency and lifetime when compared to the Examples. In addition, Comparative Example 1 to Comparative Example 7 showed degraded results of luminance and driving voltage properties compared to the Examples.

The light emitting element of one or more embodiments includes the amine compound of one or more embodiments, shows a low or reduced driving voltage, high or improved efficiency and long-life characteristics and may show high or improved luminance properties.

The amine compound of one or more embodiments may be used as a material for accomplishing an improved light emitting element having a low driving voltage, high efficiency, long-life characteristics and high luminance.

Although the embodiments of the present invention have been described, it is understood that the present disclosure should not be limited to these embodiments, but various changes and modifications can be made by one ordinary skilled in the art within the spirit and scope of the present disclosure as hereinafter claimed by the following claims and their equivalents.

What is claimed is:

1. A light emitting element, comprising:
a first electrode;
a second electrode on the first electrode; and
at least one functional layer between the first electrode and the second electrode, and comprising an amine compound represented by Formula 1: 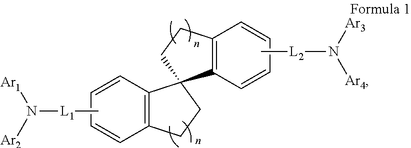
in Formula 1,
Ar₁ to Ar₄ are each independently a substituted or unsubstituted hydrocarbon ring of 6 to 30 ring-forming carbon atoms, or a substituted or unsubstituted heteroaryl group of 2 to 30 ring-forming carbon atoms,
L₁ and L₂ are each independently a direct linkage, a substituted or unsubstituted arylene group of 6 to 30 ring-forming carbon atoms, or a substituted or unsubstituted heteroarylene group of 2 to 30 ring-forming carbon atoms, and
“n” is an integer of 1 to 3.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein the hole transport region comprises a hole injection layer on the first electrode, and a hole transport layer on the hole injection layer, and
at least one selected from the hole injection layer and the hole transport layer comprises the amine compound.

4. The light emitting element of claim 2, wherein the hole transport region comprises:
a first hole transport layer adjacent to the first electrode;
a second hole transport layer on the first hole transport layer; and
a third hole transport layer on the second hole transport layer.

5. The light emitting element of claim 4, wherein the first hole transport layer and the third hole transport layer comprise the amine compound.

6. The light emitting element of claim 4, wherein the second hole transport layer comprises a compound represented by Formula H-1: 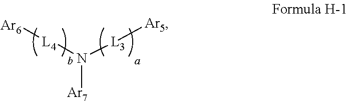
and
in Formula H-1,
Ar₅ and Ar₆ are each independently a substituted or unsubstituted aryl group of 6 to 30 ring-forming carbon atoms, or a substituted or unsubstituted heteroaryl group of 2 to 30 ring-forming carbon atoms,
Ar₇ is a substituted or unsubstituted aryl group of 6 to 30 ring-forming carbon atoms,
L₃ and L₄ are each independently a direct linkage, a substituted or unsubstituted arylene group of 6 to 30 ring-forming carbon atoms, or a substituted or unsubstituted heteroarylene group of 2 to 30 ring-forming carbon atoms, and
“a” and “b” are each independently an integer of 0 to 10.

7. The light emitting element of claim 2, wherein the hole transport region comprises a hole injection layer on the first electrode, a hole transport layer on the hole injection layer, and an electron blocking layer on the hole transport layer, and
at least one selected from the hole injection layer, the hole transport layer, and the electron blocking layer comprises the amine compound.

8. The light emitting element of claim 1, wherein the amine compound represented by Formula 1 is represented by Formula 2-1 or Formula 2-2: 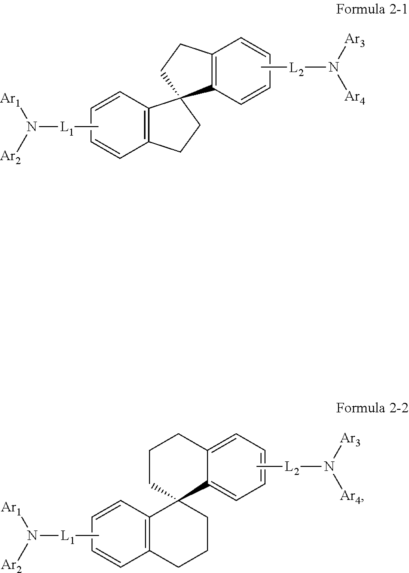
and
in Formula 2-1 and Formula 2-2,
Ar₁ to Ar₄, L₁ and L₂ are the same as defined in Formula 1.

9. The light emitting element of claim 1, wherein the amine compound represented by Formula 1 is represented by Formula 3-1 or Formula 3-2: 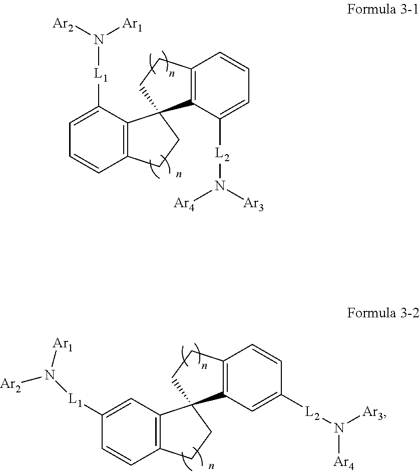
and
in Formula 3-1 and Formula 3-2,
Ar₁ to Ar₄, L₁, L₂, and “n” are the same as defined in Formula 1.

10. The light emitting element of claim 1, wherein the amine compound represented by Formula 1 is represented by Formula 4-1: 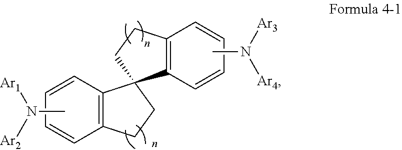
and
in Formula 4-1,
Ar₁ to Ar₄, and “n” are the same as defined in Formula 1.

11. The light emitting element of claim 1, wherein Ar₁ to Ar₄ are each independently represented by Formula 1-1a or Formula 1-1b: 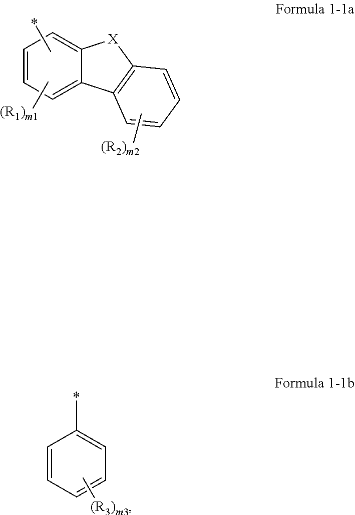
in Formula 1-1a,
X is O, S, CRₓR_{y}, or NR_{z}, and
Rₓ, R_{y}, and R_{z} are each independently a hydrogen atom, a deuterium atom, a substituted or unsubstituted alkyl group of 1 to 10 carbon atoms, or a substituted or unsubstituted aryl group of 6 to 15 ring-forming carbon atoms, and
in Formula 1-1a and Formula 1-1b,
R₁ to R₃ are each independently a hydrogen atom, a deuterium atom, a substituted or unsubstituted alkyl group of 1 to 10 carbon atoms, a substituted or unsubstituted cycloalkyl group of 6 to 15 ring-forming carbon atoms, or a substituted or unsubstituted aryl group of 6 to 15 ring-forming carbon atoms, and/or combined with an adjacent group to form a ring,
m1 is an integer of 0 to 3, m2 is an integer of 0 to 4, and
m3 is an integer of 0 to 5.

12. The light emitting element of claim 11, wherein the amine compound represented by Formula 1 is represented by Formula 5-1 or Formula 5-2: 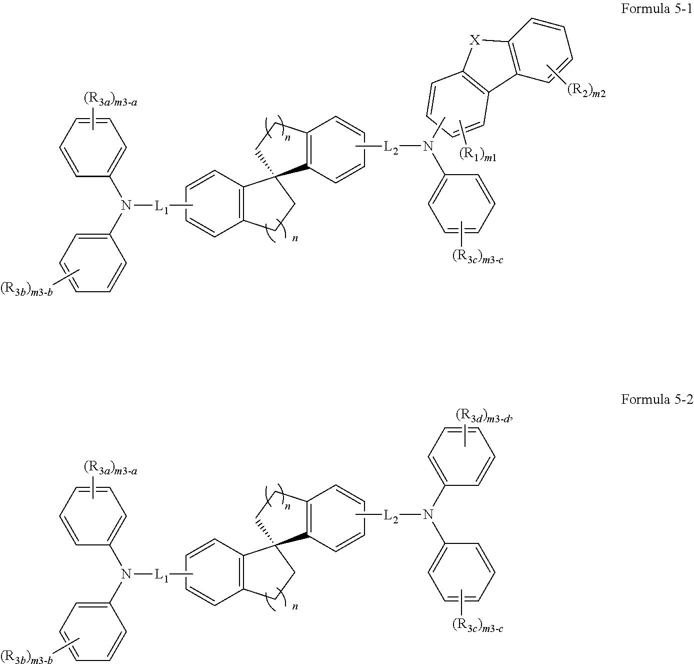
in Formula 5-1 and Formula 5-2,
L₁, L₂, and “n” are the same as defined in Formula 1,
X, R₁, R₂, m1 and m2 are the same as defined in Formula 1-1a,
R₃ₐ, R_{3b}, R_{3c}, and R_{3d} are each independently defined the same as R₃ is defined in Formula 1-1b, and
m3-a, m3-b, m3-c, and m3-d are each independently defined the same as m3 is defined in Formula 1-1b.

13. The light emitting element of claim 12, wherein the amine compound represented by Formula 5-1 is represented by Formula 5-1a or Formula 5-1b: 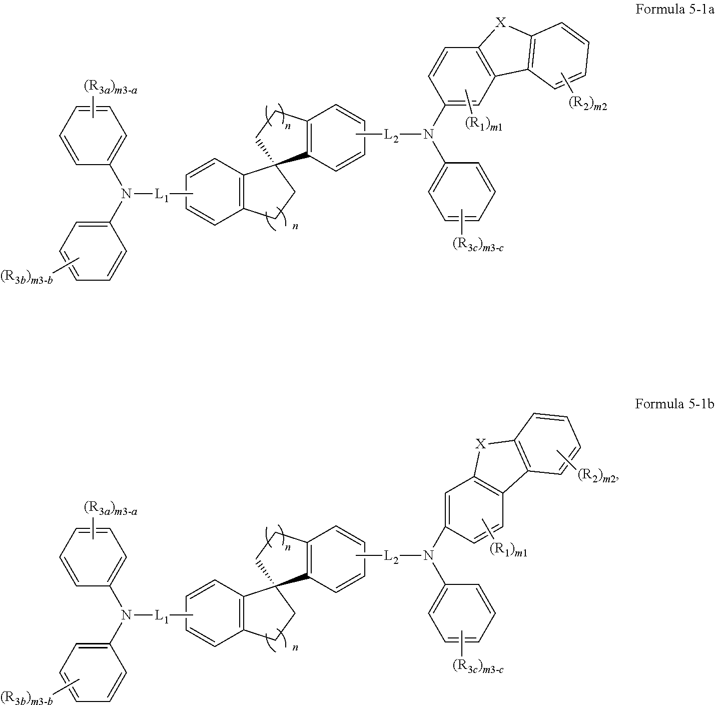
in Formula 5-1a and Formula 5-1b,
L₁, L₂, X, R₁, R₂, R₃ₐ, R_{3b}, R_{3c}, n, m1, m2, m3-a, m3-b, and m3-care the same as defined in Formula 5-1.

14. The light emitting element of claim 1, wherein the amine compound represented by Formula 1 is represented by any one selected from compounds in Compound Group 1:

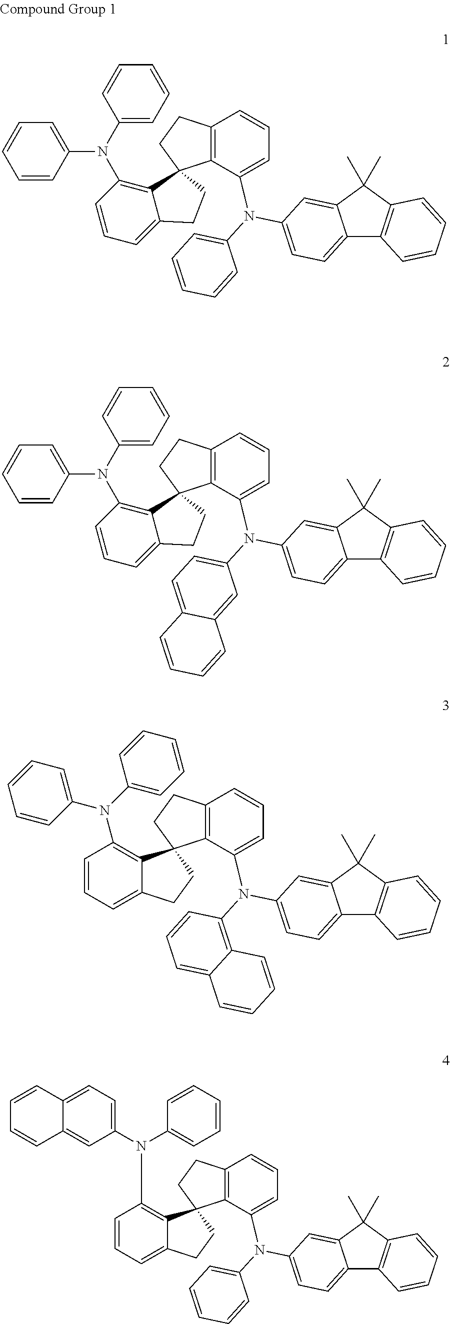 [SMILES: CC1(C)c2ccccc2-c2ccc(N(c3ccccc3)c3cccc4c3[C@]3(CCc5cccc(N(c6ccccc6)c6ccccc6)c53)CC4)cc21 ; CC1(C)c2ccccc2-c2ccc(N(c3ccc4ccccc4c3)c3cccc4c3[C@]3(CCc5cccc(N(c6ccccc6)c6ccccc6)c53)CC4)cc21 ; CC1(C)c2ccccc2-c2ccc(N(c3cccc4c3[C@]3(CCc5cccc(N(c6ccccc6)c6ccccc6)c53)CC4)c3cccc4ccccc34)cc21 ; CC1(C)c2ccccc2-c2ccc(N(c3ccccc3)c3cccc4c3[C@@]3(CC4)CCc4cccc(N(c5ccccc5)c5ccc6ccccc6c5)c43)cc21]

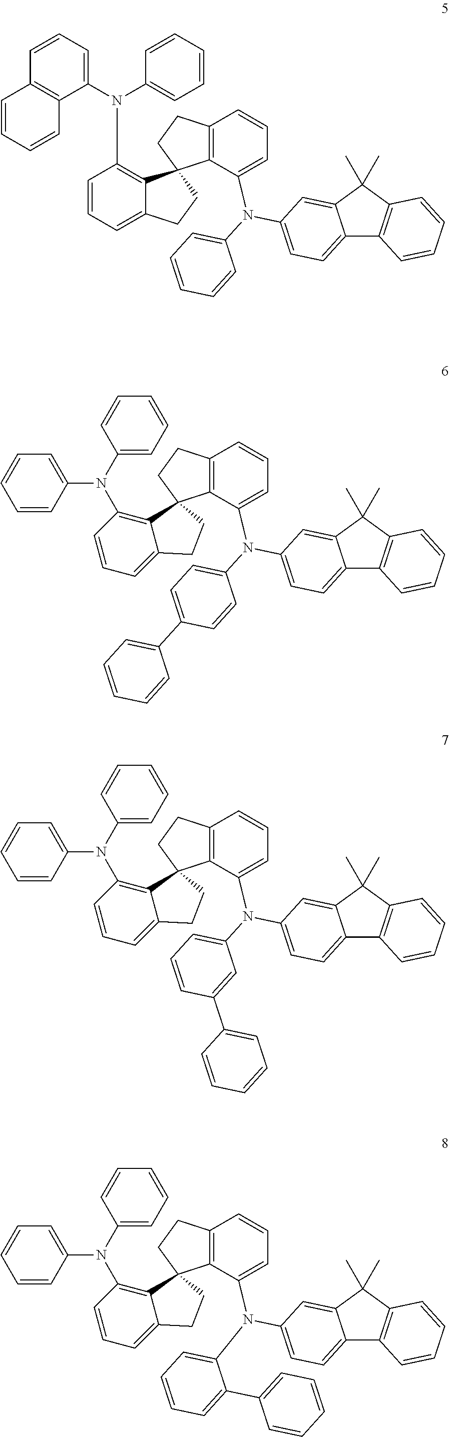 [SMILES: CC1(C)c2ccccc2-c2ccc(N(c3ccccc3)c3cccc4c3[C@@]3(CC4)CCc4cccc(N(c5ccccc5)c5cccc6ccccc56)c43)cc21 ; CC1(C)c2ccccc2-c2ccc(N(c3ccc(-c4ccccc4)cc3)c3cccc4c3[C@]3(CCc5cccc(N(c6ccccc6)c6ccccc6)c53)CC4)cc21 ; CC1(C)c2ccccc2-c2ccc(N(c3cccc(-c4ccccc4)c3)c3cccc4c3[C@]3(CCc5cccc(N(c6ccccc6)c6ccccc6)c53)CC4)cc21 ; CC1(C)c2ccccc2-c2ccc(N(c3ccccc3-c3ccccc3)c3cccc4c3[C@]3(CCc5cccc(N(c6ccccc6)c6ccccc6)c53)CC4)cc21]

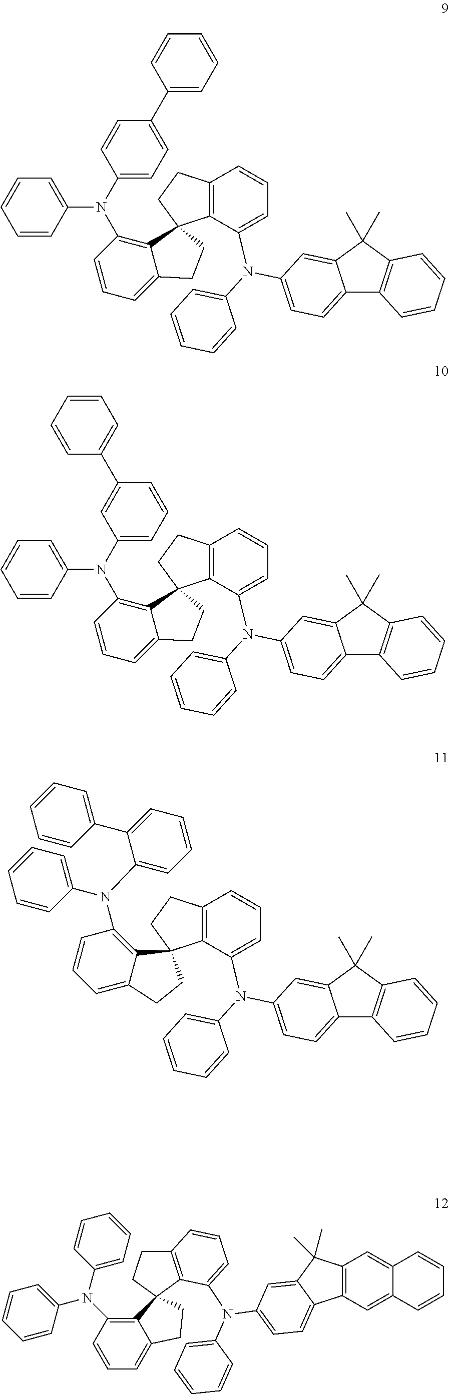 [SMILES: CC1(C)c2ccccc2-c2ccc(N(c3ccccc3)c3cccc4c3[C@]3(CCc5cccc(N(c6ccccc6)c6ccc(-c7ccccc7)cc6)c53)CC4)cc21 ; CC1(C)c2ccccc2-c2ccc(N(c3ccccc3)c3cccc4c3[C@]3(CCc5cccc(N(c6ccccc6)c6cccc(-c7ccccc7)c6)c53)CC4)cc21 ; CC1(C)c2ccccc2-c2ccc(N(c3ccccc3)c3cccc4c3[C@@]3(CC4)CCc4cccc(N(c5ccccc5)c5ccccc5-c5ccccc5)c43)cc21 ; CC1(C)c2cc(N(c3ccccc3)c3cccc4c3[C@]3(CCc5cccc(N(c6ccccc6)c6ccccc6)c53)CC4)ccc2-c2cc3ccccc3cc21]

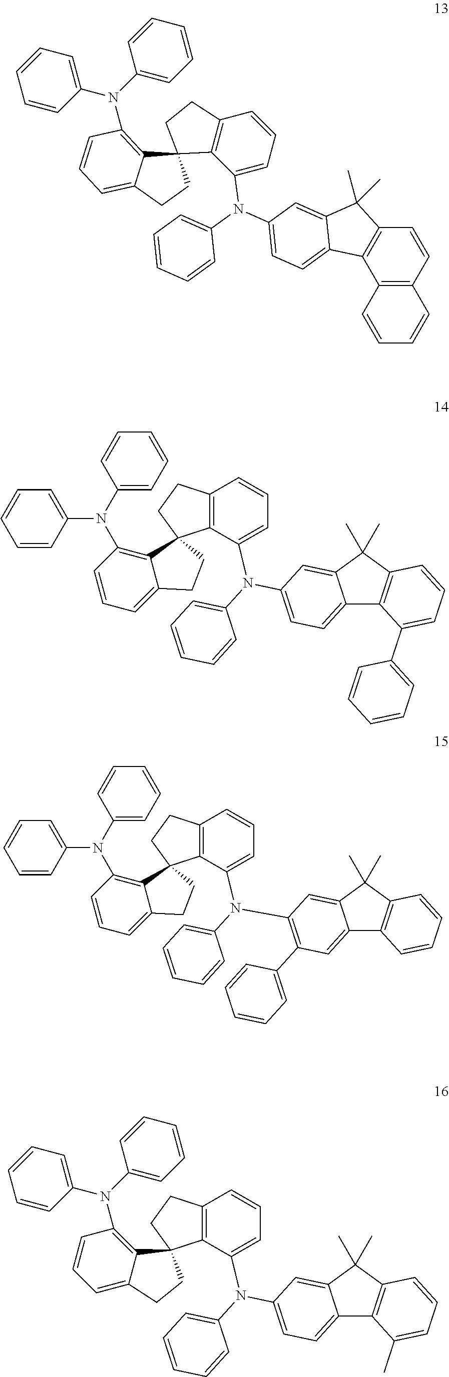 [SMILES: CC1(C)c2cc(N(c3ccccc3)c3cccc4c3[C@]3(CCc5cccc(N(c6ccccc6)c6ccccc6)c53)CC4)ccc2-c2c1ccc1ccccc21 ; CC1(C)c2cc(N(c3ccccc3)c3cccc4c3[C@]3(CCc5cccc(N(c6ccccc6)c6ccccc6)c53)CC4)ccc2-c2c(-c3ccccc3)cccc21 ; CC1(C)c2ccccc2-c2cc(-c3ccccc3)c(N(c3ccccc3)c3cccc4c3[C@]3(CCc5cccc(N(c6ccccc6)c6ccccc6)c53)CC4)cc21 ; Cc1cccc2c1-c1ccc(N(c3ccccc3)c3cccc4c3[C@]3(CCc5cccc(N(c6ccccc6)c6ccccc6)c53)CC4)cc1C2(C)C]

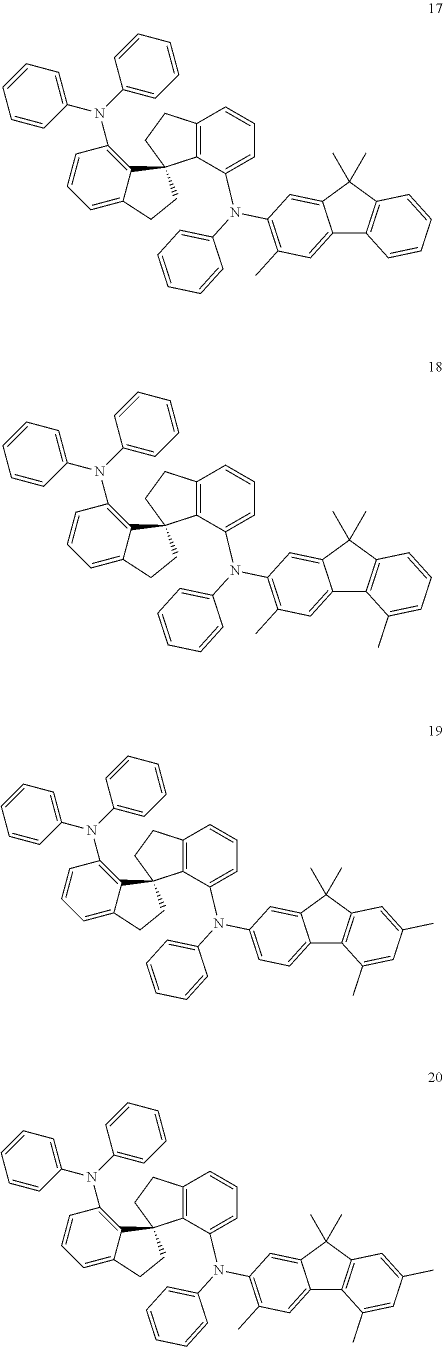 [SMILES: Cc1cc2c(cc1N(c1ccccc1)c1cccc3c1[C@]1(CCc4cccc(N(c5ccccc5)c5ccccc5)c41)CC3)C(C)(C)c1ccccc1-2 ; Cc1cc2c(cc1N(c1ccccc1)c1cccc3c1[C@]1(CCc4cccc(N(c5ccccc5)c5ccccc5)c41)CC3)C(C)(C)c1cccc(C)c1-2 ; Cc1cc(C)c2c(c1)C(C)(C)c1cc(N(c3ccccc3)c3cccc4c3[C@]3(CCc5cccc(N(c6ccccc6)c6ccccc6)c53)CC4)ccc1-2 ; Cc1cc(C)c2c(c1)C(C)(C)c1cc(N(c3ccccc3)c3cccc4c3[C@]3(CCc5cccc(N(c6ccccc6)c6ccccc6)c53)CC4)c(C)cc1-2]

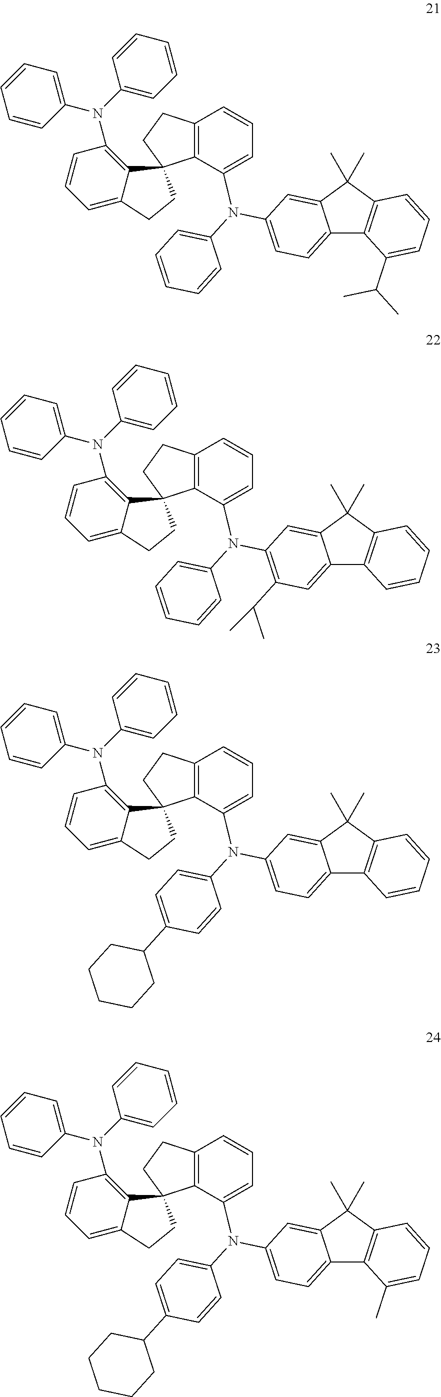 [SMILES: CC(C)c1cccc2c1-c1ccc(N(c3ccccc3)c3cccc4c3[C@]3(CCc5cccc(N(c6ccccc6)c6ccccc6)c53)CC4)cc1C2(C)C ; CC(C)c1cc2c(cc1N(c1ccccc1)c1cccc3c1[C@]1(CCc4cccc(N(c5ccccc5)c5ccccc5)c41)CC3)C(C)(C)c1ccccc1-2 ; CC1(C)c2ccccc2-c2ccc(N(c3ccc(C4CCCCC4)cc3)c3cccc4c3[C@]3(CCc5cccc(N(c6ccccc6)c6ccccc6)c53)CC4)cc21 ; Cc1cccc2c1-c1ccc(N(c3ccc(C4CCCCC4)cc3)c3cccc4c3[C@]3(CCc5cccc(N(c6ccccc6)c6ccccc6)c53)CC4)cc1C2(C)C]

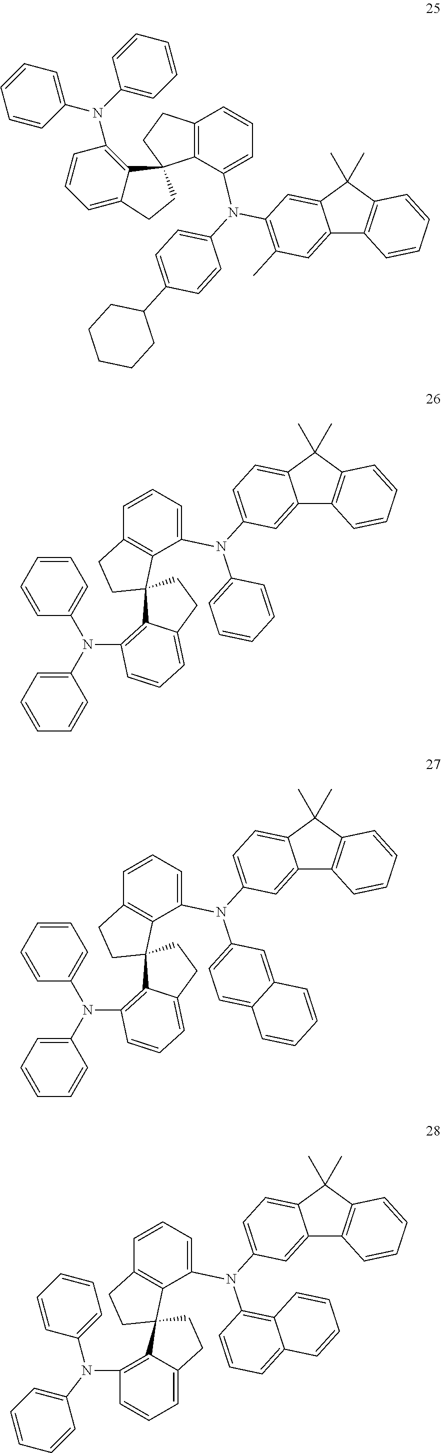 [SMILES: Cc1cc2c(cc1N(c1ccc(C3CCCCC3)cc1)c1cccc3c1[C@]1(CCc4cccc(N(c5ccccc5)c5ccccc5)c41)CC3)C(C)(C)c1ccccc1-2 ; CC1(C)c2ccccc2-c2cc(N(c3ccccc3)c3cccc4c3[C@]3(CCc5cccc(N(c6ccccc6)c6ccccc6)c53)CC4)ccc21 ; CC1(C)c2ccccc2-c2cc(N(c3ccc4ccccc4c3)c3cccc4c3[C@]3(CCc5cccc(N(c6ccccc6)c6ccccc6)c53)CC4)ccc21 ; CC1(C)c2ccccc2-c2cc(N(c3cccc4c3[C@]3(CCc5cccc(N(c6ccccc6)c6ccccc6)c53)CC4)c3cccc4ccccc34)ccc21]

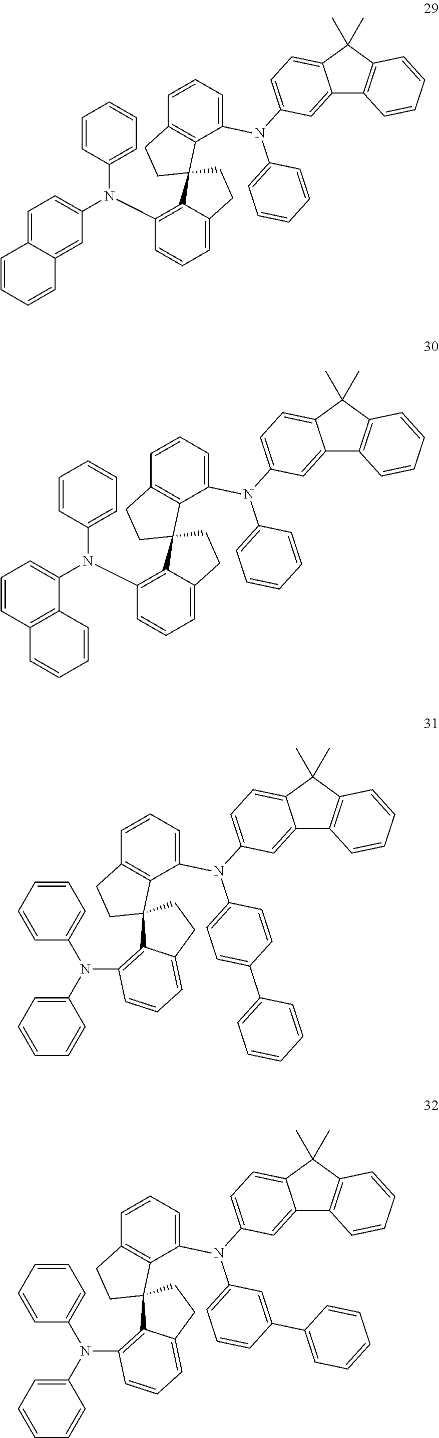 [SMILES: CC1(C)c2ccccc2-c2cc(N(c3ccccc3)c3cccc4c3[C@@]3(CC4)CCc4cccc(N(c5ccccc5)c5ccc6ccccc6c5)c43)ccc21 ; CC1(C)c2ccccc2-c2cc(N(c3ccccc3)c3cccc4c3[C@@]3(CC4)CCc4cccc(N(c5ccccc5)c5cccc6ccccc56)c43)ccc21 ; CC1(C)c2ccccc2-c2cc(N(c3ccc(-c4ccccc4)cc3)c3cccc4c3[C@]3(CCc5cccc(N(c6ccccc6)c6ccccc6)c53)CC4)ccc21 ; CC1(C)c2ccccc2-c2cc(N(c3cccc(-c4ccccc4)c3)c3cccc4c3[C@]3(CCc5cccc(N(c6ccccc6)c6ccccc6)c53)CC4)ccc21]

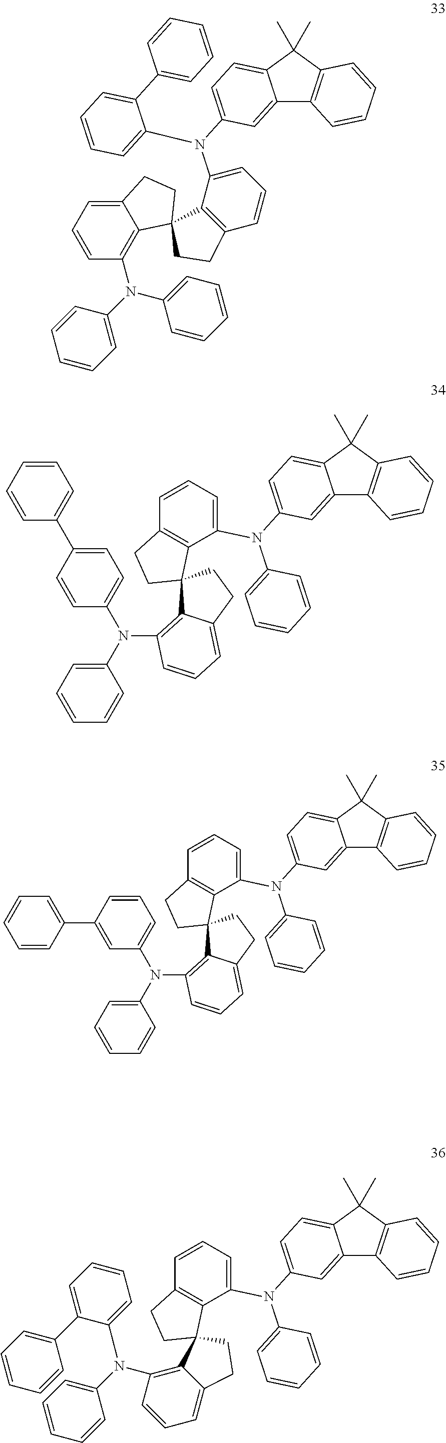

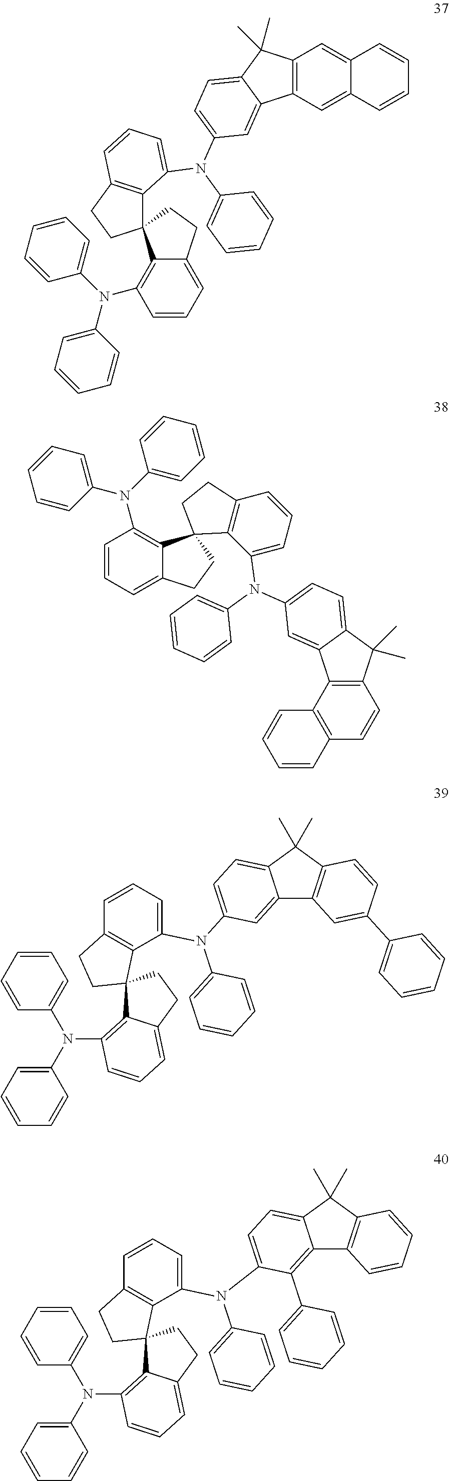 [SMILES: CC1(C)c2ccc(N(c3ccccc3)c3cccc4c3[C@]3(CCc5cccc(N(c6ccccc6)c6ccccc6)c53)CC4)cc2-c2cc3ccccc3cc21 ; CC1(C)c2ccc(N(c3ccccc3)c3cccc4c3[C@]3(CCc5cccc(N(c6ccccc6)c6ccccc6)c53)CC4)cc2-c2c1ccc1ccccc21 ; CC1(C)c2ccc(-c3ccccc3)cc2-c2cc(N(c3ccccc3)c3cccc4c3[C@]3(CCc5cccc(N(c6ccccc6)c6ccccc6)c53)CC4)ccc21 ; CC1(C)c2ccccc2-c2c1ccc(N(c1ccccc1)c1cccc3c1[C@]1(CCc4cccc(N(c5ccccc5)c5ccccc5)c41)CC3)c2-c1ccccc1]

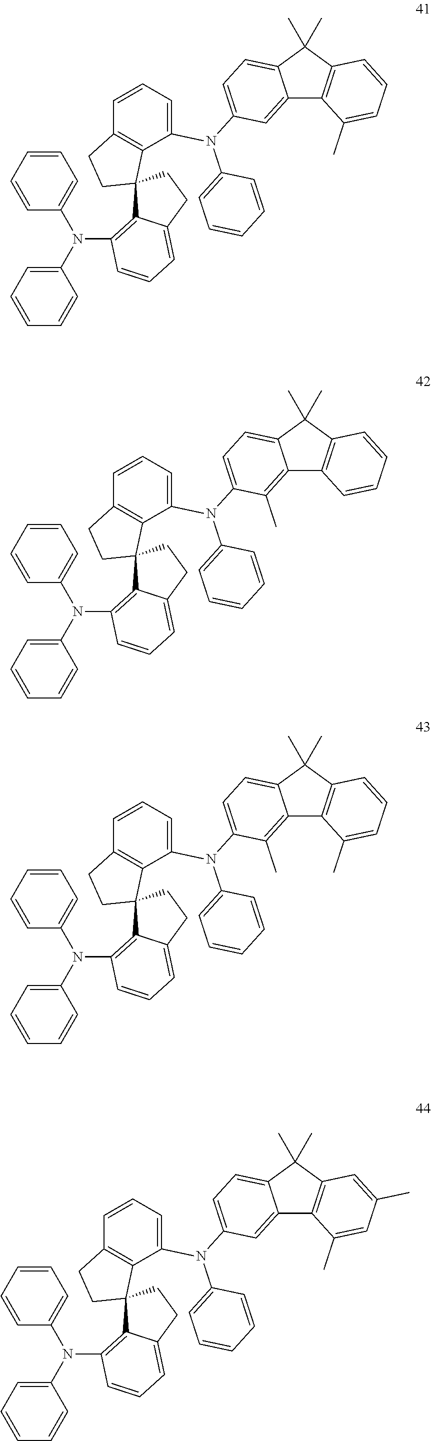 [SMILES: Cc1cccc2c1-c1cc(N(c3ccccc3)c3cccc4c3[C@]3(CCc5cccc(N(c6ccccc6)c6ccccc6)c53)CC4)ccc1C2(C)C ; Cc1c(N(c2ccccc2)c2cccc3c2[C@]2(CCc4cccc(N(c5ccccc5)c5ccccc5)c42)CC3)ccc2c1-c1ccccc1C2(C)C ; Cc1cccc2c1-c1c(ccc(N(c3ccccc3)c3cccc4c3[C@]3(CCc5cccc(N(c6ccccc6)c6ccccc6)c53)CC4)c1C)C2(C)C ; Cc1cc(C)c2c(c1)C(C)(C)c1ccc(N(c3ccccc3)c3cccc4c3[C@]3(CCc5cccc(N(c6ccccc6)c6ccccc6)c53)CC4)cc1-2]

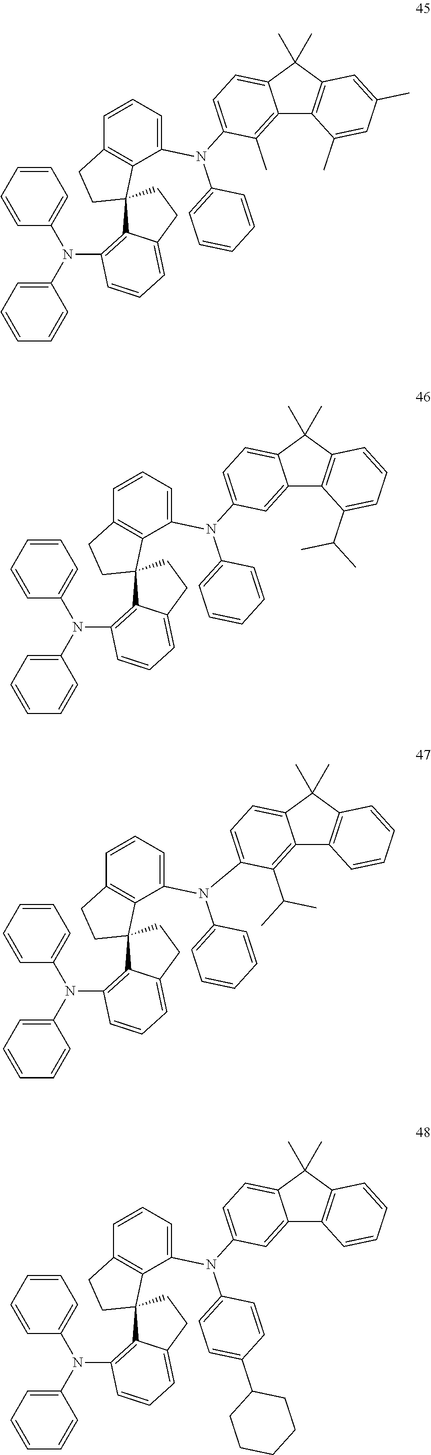 [SMILES: Cc1cc(C)c2c(c1)C(C)(C)c1ccc(N(c3ccccc3)c3cccc4c3[C@]3(CCc5cccc(N(c6ccccc6)c6ccccc6)c53)CC4)c(C)c1-2 ; CC(C)c1cccc2c1-c1cc(N(c3ccccc3)c3cccc4c3[C@]3(CCc5cccc(N(c6ccccc6)c6ccccc6)c53)CC4)ccc1C2(C)C ; CC(C)c1c(N(c2ccccc2)c2cccc3c2[C@]2(CCc4cccc(N(c5ccccc5)c5ccccc5)c42)CC3)ccc2c1-c1ccccc1C2(C)C ; CC1(C)c2ccccc2-c2cc(N(c3ccc(C4CCCCC4)cc3)c3cccc4c3[C@]3(CCc5cccc(N(c6ccccc6)c6ccccc6)c53)CC4)ccc21]

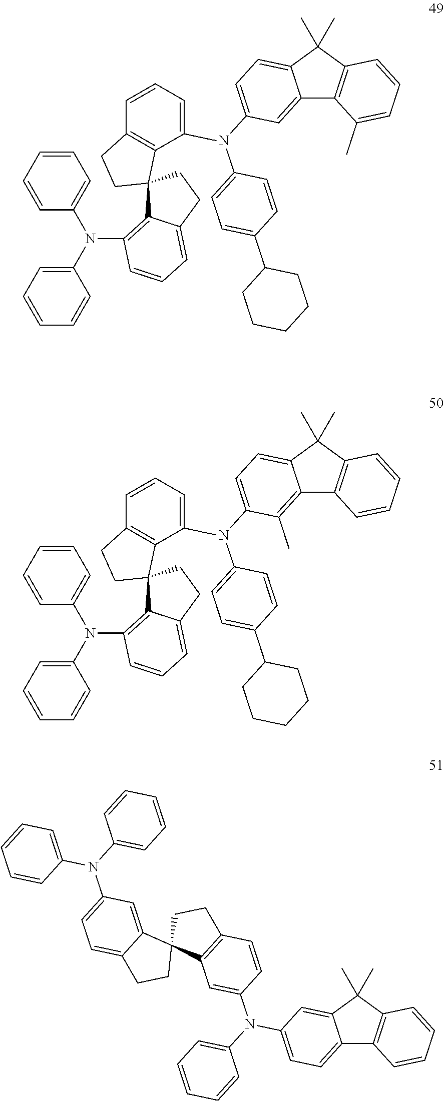 [SMILES: Cc1cccc2c1-c1cc(N(c3ccc(C4CCCCC4)cc3)c3cccc4c3[C@]3(CCc5cccc(N(c6ccccc6)c6ccccc6)c53)CC4)ccc1C2(C)C ; Cc1c(N(c2ccc(C3CCCCC3)cc2)c2cccc3c2[C@]2(CCc4cccc(N(c5ccccc5)c5ccccc5)c42)CC3)ccc2c1-c1ccccc1C2(C)C ; CC1(C)c2ccccc2-c2ccc(N(c3ccccc3)c3ccc4c(c3)[C@]3(CCc5ccc(N(c6ccccc6)c6ccccc6)cc53)CC4)cc21]

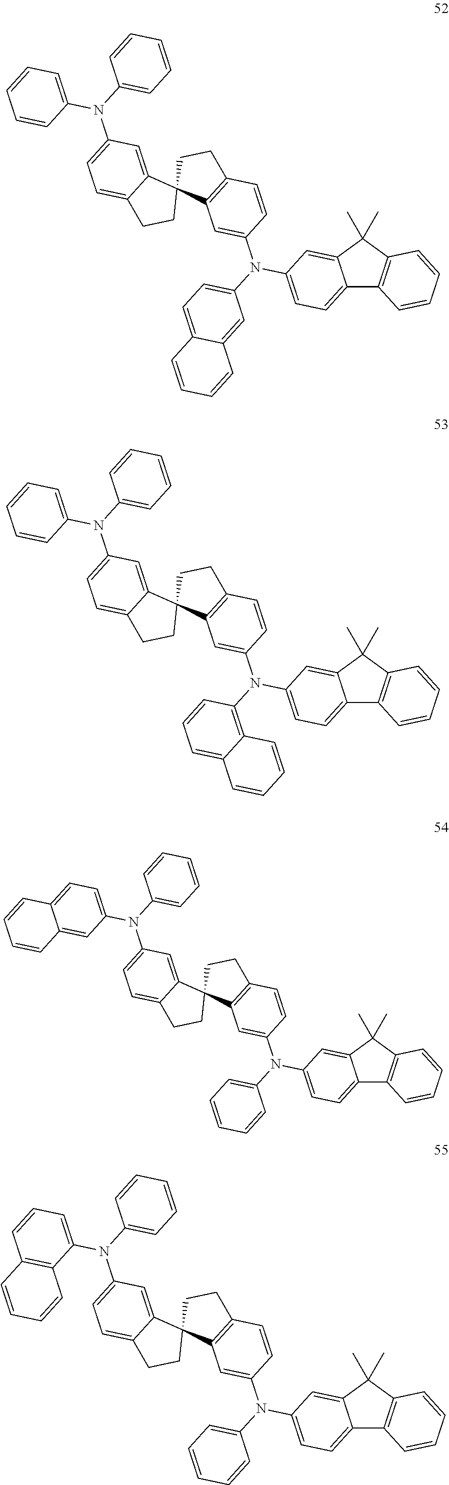 [SMILES: CC1(C)c2ccccc2-c2ccc(N(c3ccc4c(c3)[C@]3(CCc5ccc(N(c6ccccc6)c6ccccc6)cc53)CC4)c3ccc4ccccc4c3)cc21 ; CC1(C)c2ccccc2-c2ccc(N(c3ccc4c(c3)[C@]3(CCc5ccc(N(c6ccccc6)c6ccccc6)cc53)CC4)c3cccc4ccccc34)cc21 ; CC1(C)c2ccccc2-c2ccc(N(c3ccccc3)c3ccc4c(c3)[C@@]3(CC4)CCc4ccc(N(c5ccccc5)c5ccc6ccccc6c5)cc43)cc21 ; CC1(C)c2ccccc2-c2ccc(N(c3ccccc3)c3ccc4c(c3)[C@@]3(CC4)CCc4ccc(N(c5ccccc5)c5cccc6ccccc56)cc43)cc21]

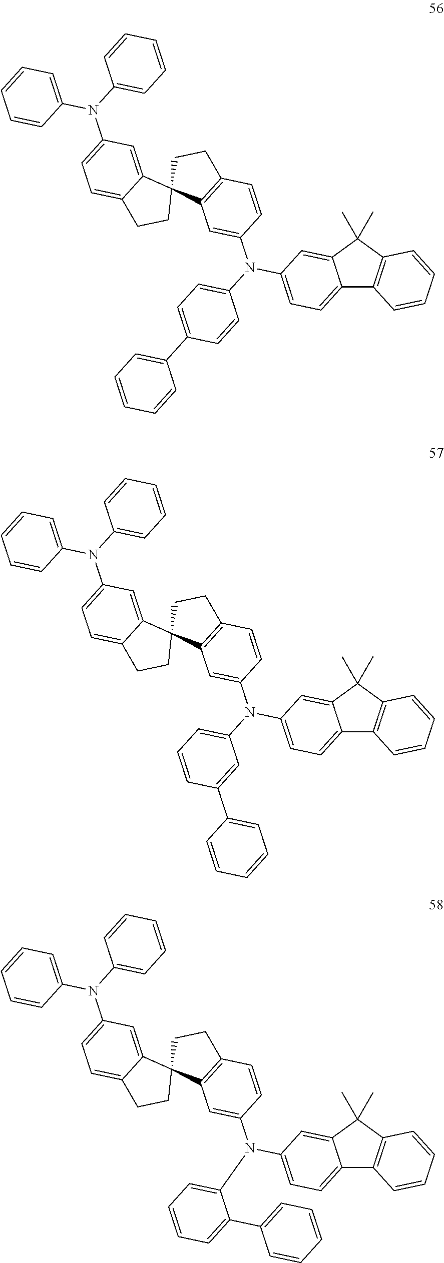 [SMILES: CC1(C)c2ccccc2-c2ccc(N(c3ccc(-c4ccccc4)cc3)c3ccc4c(c3)[C@]3(CCc5ccc(N(c6ccccc6)c6ccccc6)cc53)CC4)cc21 ; CC1(C)c2ccccc2-c2ccc(N(c3cccc(-c4ccccc4)c3)c3ccc4c(c3)[C@]3(CCc5ccc(N(c6ccccc6)c6ccccc6)cc53)CC4)cc21 ; CC1(C)c2ccccc2-c2ccc(N(c3ccc4c(c3)[C@]3(CCc5ccc(N(c6ccccc6)c6ccccc6)cc53)CC4)c3ccccc3-c3ccccc3)cc21]

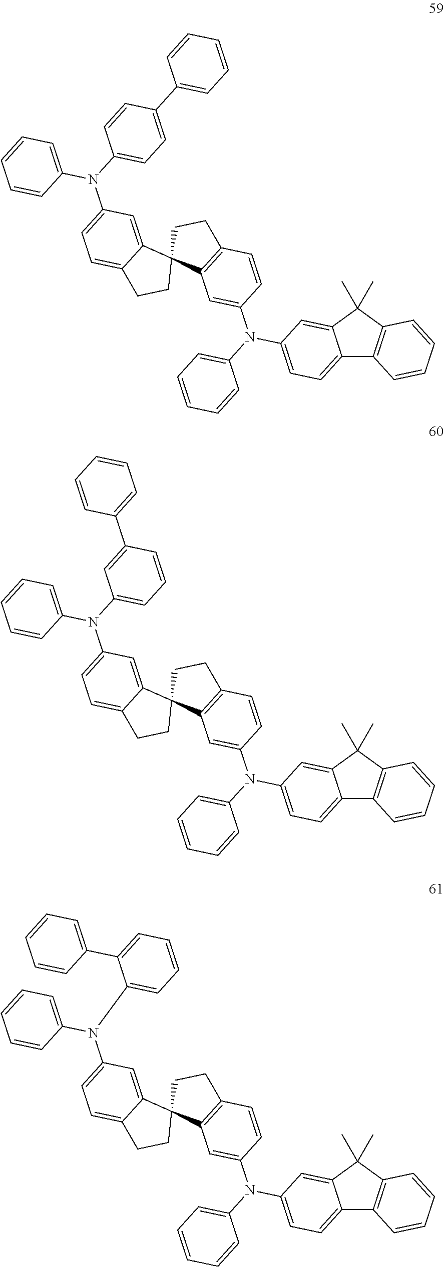 [SMILES: CC1(C)c2ccccc2-c2ccc(N(c3ccccc3)c3ccc4c(c3)[C@]3(CCc5ccc(N(c6ccccc6)c6ccc(-c7ccccc7)cc6)cc53)CC4)cc21 ; CC1(C)c2ccccc2-c2ccc(N(c3ccccc3)c3ccc4c(c3)[C@]3(CCc5ccc(N(c6ccccc6)c6cccc(-c7ccccc7)c6)cc53)CC4)cc21 ; CC1(C)c2ccccc2-c2ccc(N(c3ccccc3)c3ccc4c(c3)[C@@]3(CC4)CCc4ccc(N(c5ccccc5)c5ccccc5-c5ccccc5)cc43)cc21]

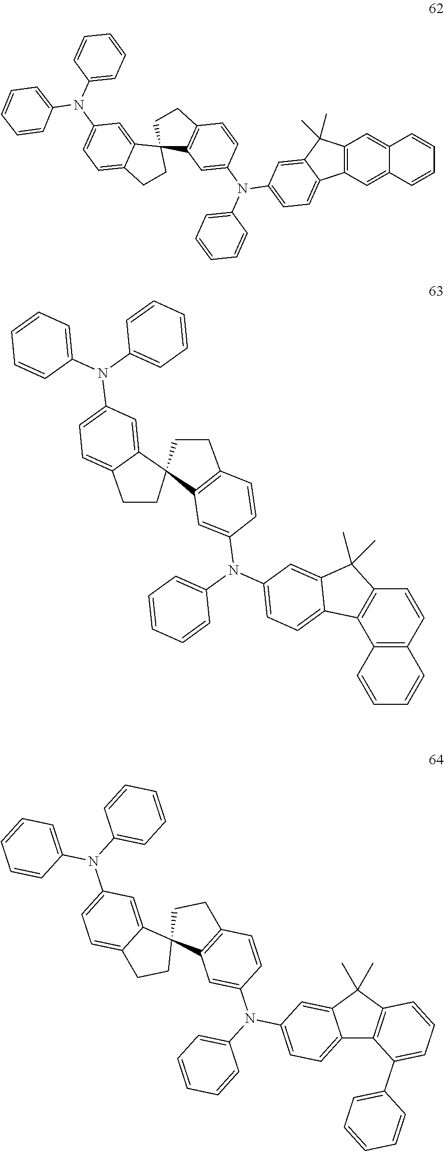 [SMILES: CC1(C)c2cc(N(c3ccccc3)c3ccc4c(c3)[C@]3(CCc5ccc(N(c6ccccc6)c6ccccc6)cc53)CC4)ccc2-c2cc3ccccc3cc21 ; CC1(C)c2cc(N(c3ccccc3)c3ccc4c(c3)[C@]3(CCc5ccc(N(c6ccccc6)c6ccccc6)cc53)CC4)ccc2-c2c1ccc1ccccc21 ; CC1(C)c2cc(N(c3ccccc3)c3ccc4c(c3)[C@]3(CCc5ccc(N(c6ccccc6)c6ccccc6)cc53)CC4)ccc2-c2c(-c3ccccc3)cccc21]

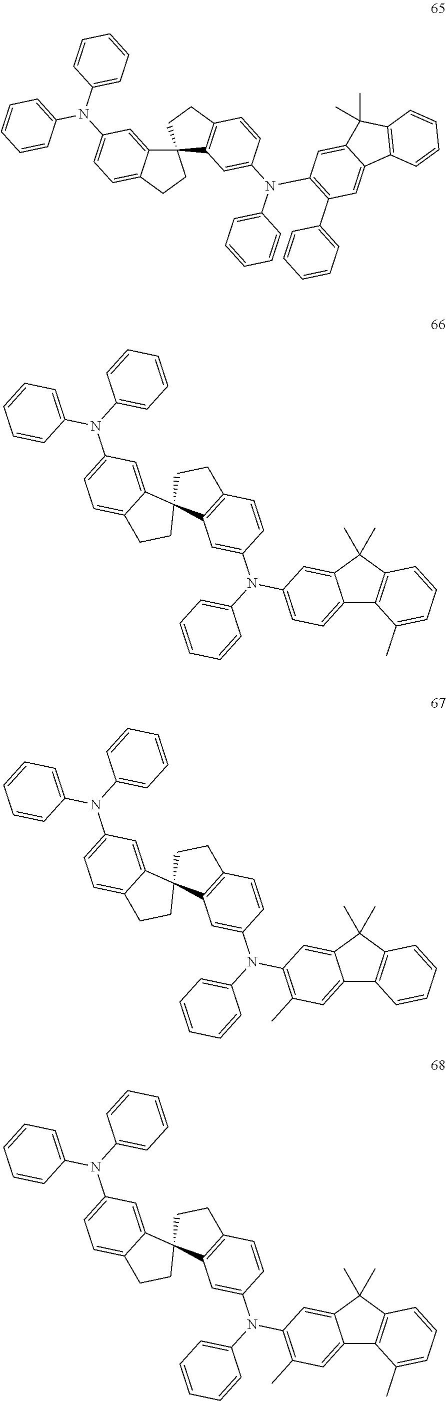 [SMILES: CC1(C)c2ccccc2-c2cc(-c3ccccc3)c(N(c3ccccc3)c3ccc4c(c3)[C@]3(CCc5ccc(N(c6ccccc6)c6ccccc6)cc53)CC4)cc21 ; Cc1cccc2c1-c1ccc(N(c3ccccc3)c3ccc4c(c3)[C@]3(CCc5ccc(N(c6ccccc6)c6ccccc6)cc53)CC4)cc1C2(C)C ; Cc1cc2c(cc1N(c1ccccc1)c1ccc3c(c1)[C@]1(CCc4ccc(N(c5ccccc5)c5ccccc5)cc41)CC3)C(C)(C)c1ccccc1-2 ; Cc1cc2c(cc1N(c1ccccc1)c1ccc3c(c1)[C@]1(CCc4ccc(N(c5ccccc5)c5ccccc5)cc41)CC3)C(C)(C)c1cccc(C)c1-2]

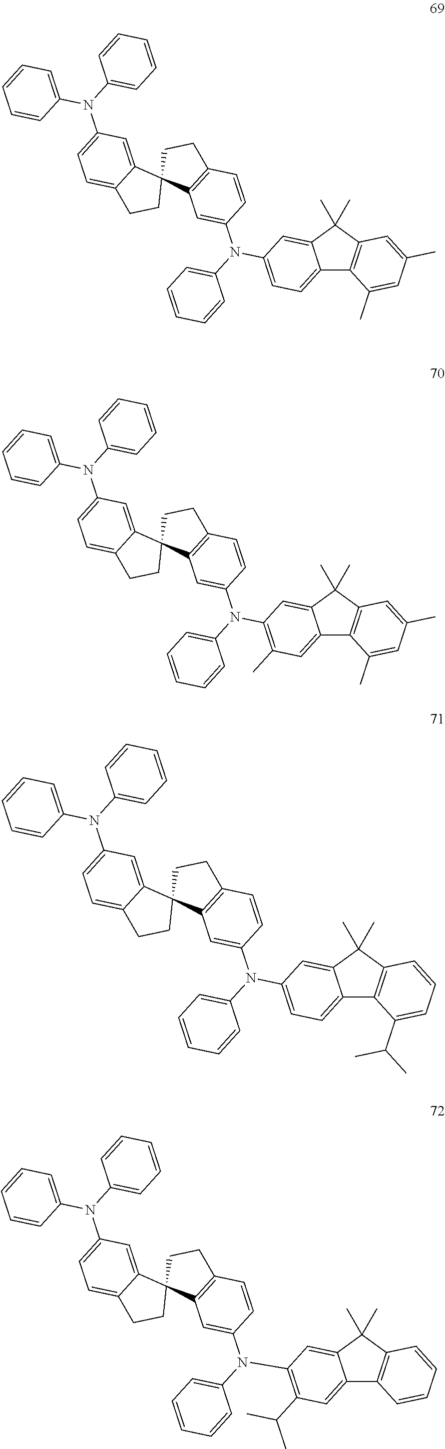 [SMILES: Cc1cc(C)c2c(c1)C(C)(C)c1cc(N(c3ccccc3)c3ccc4c(c3)[C@]3(CCc5ccc(N(c6ccccc6)c6ccccc6)cc53)CC4)ccc1-2 ; Cc1cc(C)c2c(c1)C(C)(C)c1cc(N(c3ccccc3)c3ccc4c(c3)[C@]3(CCc5ccc(N(c6ccccc6)c6ccccc6)cc53)CC4)c(C)cc1-2 ; CC(C)c1cccc2c1-c1ccc(N(c3ccccc3)c3ccc4c(c3)[C@]3(CCc5ccc(N(c6ccccc6)c6ccccc6)cc53)CC4)cc1C2(C)C ; CC(C)c1cc2c(cc1N(c1ccccc1)c1ccc3c(c1)[C@]1(CCc4ccc(N(c5ccccc5)c5ccccc5)cc41)CC3)C(C)(C)c1ccccc1-2]

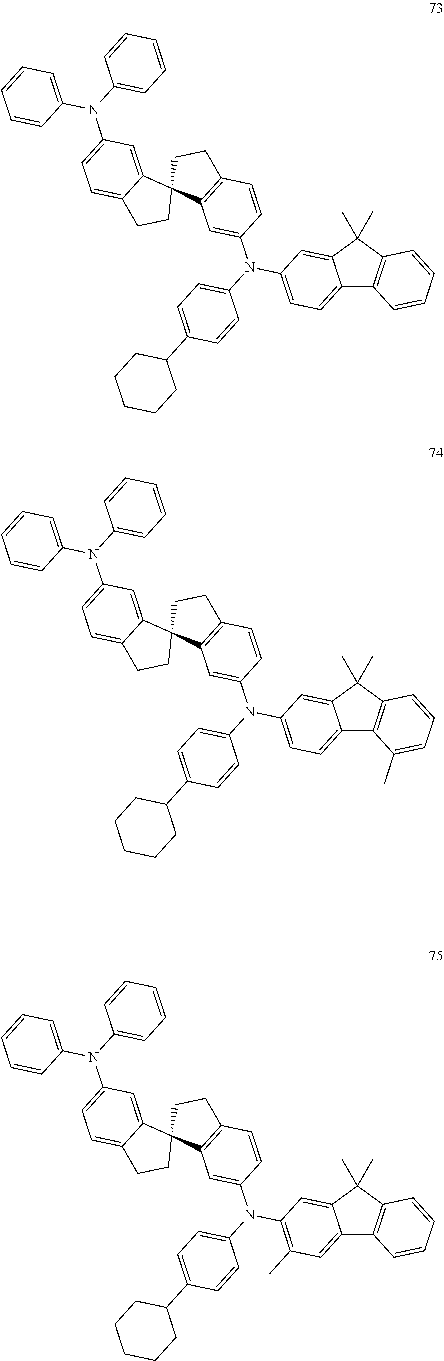 [SMILES: CC1(C)c2ccccc2-c2ccc(N(c3ccc(C4CCCCC4)cc3)c3ccc4c(c3)[C@]3(CCc5ccc(N(c6ccccc6)c6ccccc6)cc53)CC4)cc21 ; Cc1cccc2c1-c1ccc(N(c3ccc(C4CCCCC4)cc3)c3ccc4c(c3)[C@]3(CCc5ccc(N(c6ccccc6)c6ccccc6)cc53)CC4)cc1C2(C)C ; Cc1cc2c(cc1N(c1ccc(C3CCCCC3)cc1)c1ccc3c(c1)[C@]1(CCc4ccc(N(c5ccccc5)c5ccccc5)cc41)CC3)C(C)(C)c1ccccc1-2]

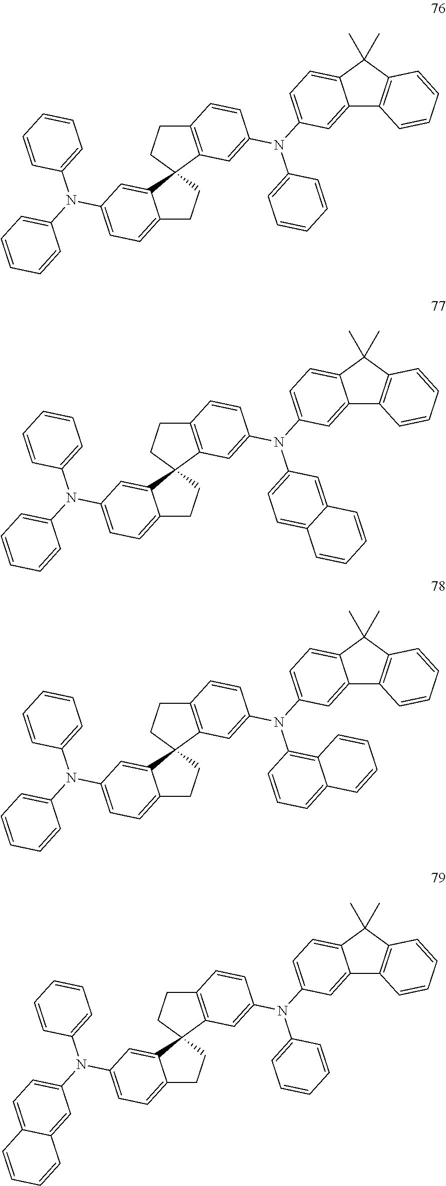 [SMILES: CC1(C)c2ccccc2-c2cc(N(c3ccccc3)c3ccc4c(c3)[C@]3(CCc5ccc(N(c6ccccc6)c6ccccc6)cc53)CC4)ccc21 ; CC1(C)c2ccccc2-c2cc(N(c3ccc4c(c3)[C@]3(CCc5ccc(N(c6ccccc6)c6ccccc6)cc53)CC4)c3ccc4ccccc4c3)ccc21 ; CC1(C)c2ccccc2-c2cc(N(c3ccc4c(c3)[C@]3(CCc5ccc(N(c6ccccc6)c6ccccc6)cc53)CC4)c3cccc4ccccc34)ccc21 ; CC1(C)c2ccccc2-c2cc(N(c3ccccc3)c3ccc4c(c3)[C@@]3(CC4)CCc4ccc(N(c5ccccc5)c5ccc6ccccc6c5)cc43)ccc21]

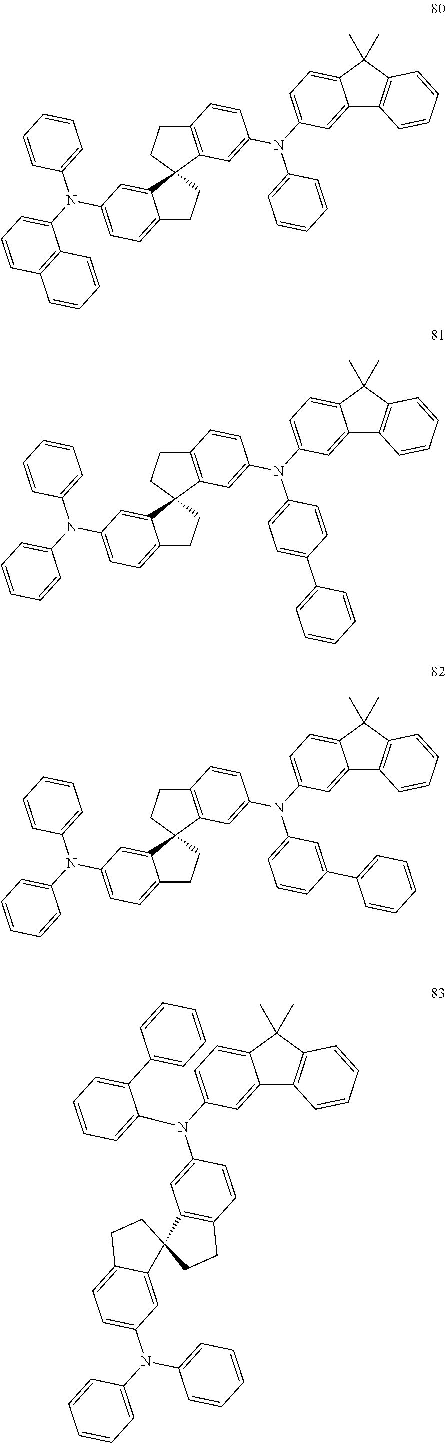 [SMILES: CC1(C)c2ccccc2-c2cc(N(c3ccccc3)c3ccc4c(c3)[C@@]3(CC4)CCc4ccc(N(c5ccccc5)c5cccc6ccccc56)cc43)ccc21 ; CC1(C)c2ccccc2-c2cc(N(c3ccc(-c4ccccc4)cc3)c3ccc4c(c3)[C@]3(CCc5ccc(N(c6ccccc6)c6ccccc6)cc53)CC4)ccc21 ; CC1(C)c2ccccc2-c2cc(N(c3cccc(-c4ccccc4)c3)c3ccc4c(c3)[C@]3(CCc5ccc(N(c6ccccc6)c6ccccc6)cc53)CC4)ccc21 ; CC1(C)c2ccccc2-c2cc(N(c3ccc4c(c3)[C@]3(CCc5ccc(N(c6ccccc6)c6ccccc6)cc53)CC4)c3ccccc3-c3ccccc3)ccc21]

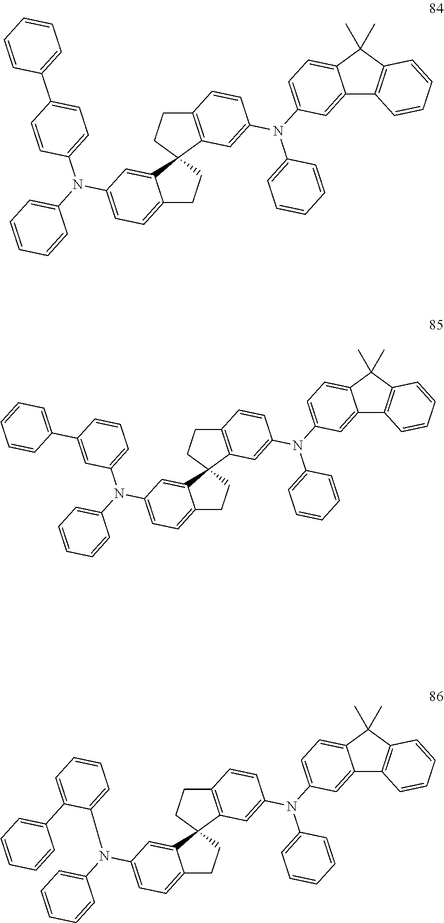 [SMILES: CC1(C)c2ccccc2-c2cc(N(c3ccccc3)c3ccc4c(c3)[C@]3(CCc5ccc(N(c6ccccc6)c6ccc(-c7ccccc7)cc6)cc53)CC4)ccc21 ; CC1(C)c2ccccc2-c2cc(N(c3ccccc3)c3ccc4c(c3)[C@]3(CCc5ccc(N(c6ccccc6)c6cccc(-c7ccccc7)c6)cc53)CC4)ccc21 ; CC1(C)c2ccccc2-c2cc(N(c3ccccc3)c3ccc4c(c3)[C@@]3(CC4)CCc4ccc(N(c5ccccc5)c5ccccc5-c5ccccc5)cc43)ccc21]

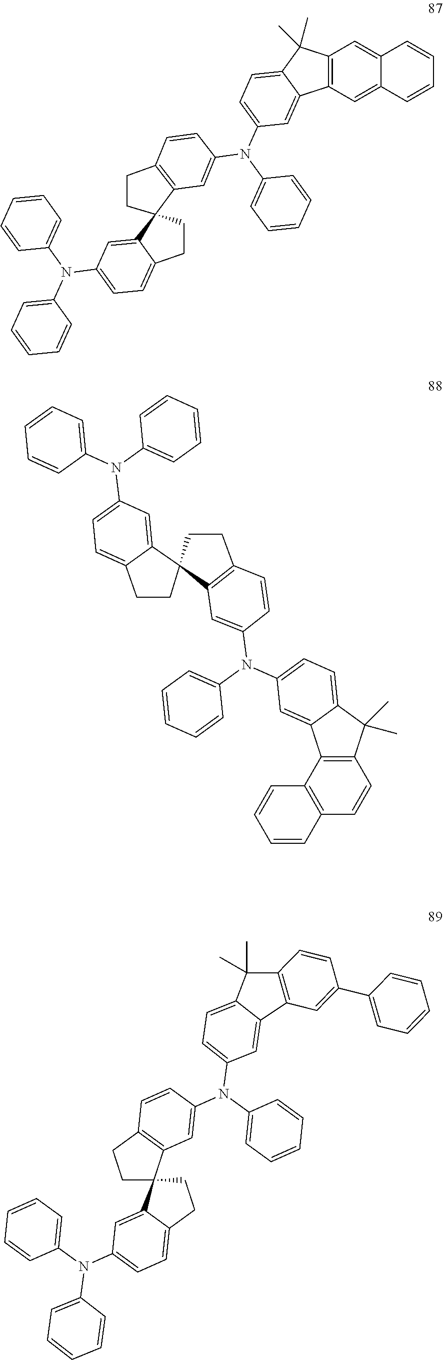 [SMILES: CC1(C)c2ccc(N(c3ccccc3)c3ccc4c(c3)[C@]3(CCc5ccc(N(c6ccccc6)c6ccccc6)cc53)CC4)cc2-c2cc3ccccc3cc21 ; CC1(C)c2ccc(N(c3ccccc3)c3ccc4c(c3)[C@]3(CCc5ccc(N(c6ccccc6)c6ccccc6)cc53)CC4)cc2-c2c1ccc1ccccc21 ; CC1(C)c2ccc(-c3ccccc3)cc2-c2cc(N(c3ccccc3)c3ccc4c(c3)[C@]3(CCc5ccc(N(c6ccccc6)c6ccccc6)cc53)CC4)ccc21]

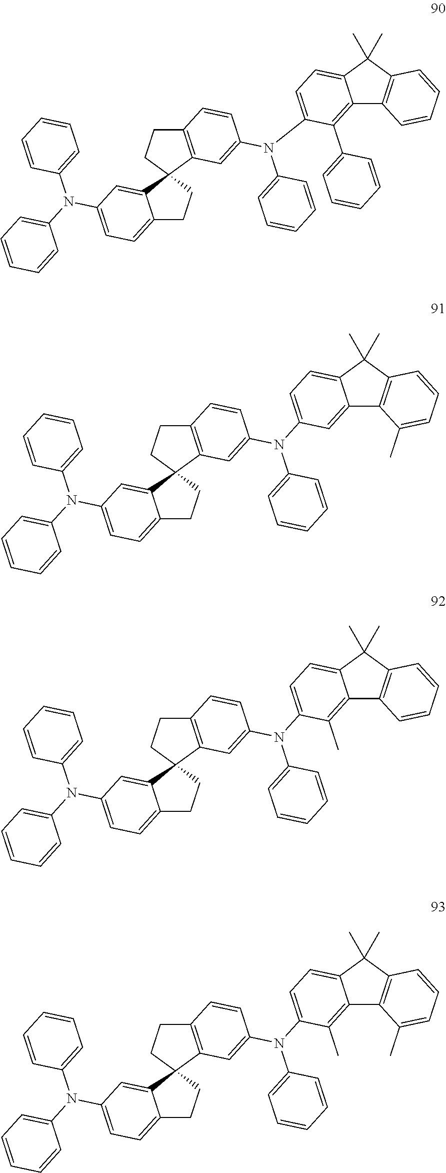 [SMILES: CC1(C)c2ccccc2-c2c1ccc(N(c1ccccc1)c1ccc3c(c1)[C@]1(CCc4ccc(N(c5ccccc5)c5ccccc5)cc41)CC3)c2-c1ccccc1 ; Cc1cccc2c1-c1cc(N(c3ccccc3)c3ccc4c(c3)[C@]3(CCc5ccc(N(c6ccccc6)c6ccccc6)cc53)CC4)ccc1C2(C)C ; Cc1c(N(c2ccccc2)c2ccc3c(c2)[C@]2(CCc4ccc(N(c5ccccc5)c5ccccc5)cc42)CC3)ccc2c1-c1ccccc1C2(C)C ; Cc1cccc2c1-c1c(ccc(N(c3ccccc3)c3ccc4c(c3)[C@]3(CCc5ccc(N(c6ccccc6)c6ccccc6)cc53)CC4)c1C)C2(C)C]

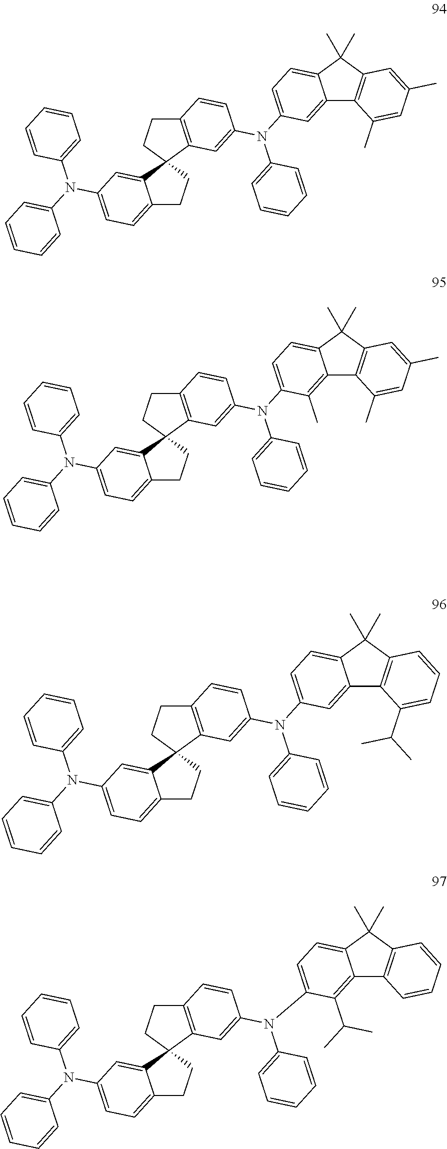 [SMILES: Cc1cc(C)c2c(c1)C(C)(C)c1ccc(N(c3ccccc3)c3ccc4c(c3)[C@]3(CCc5ccc(N(c6ccccc6)c6ccccc6)cc53)CC4)cc1-2 ; Cc1cc(C)c2c(c1)C(C)(C)c1ccc(N(c3ccccc3)c3ccc4c(c3)[C@]3(CCc5ccc(N(c6ccccc6)c6ccccc6)cc53)CC4)c(C)c1-2 ; CC(C)c1cccc2c1-c1cc(N(c3ccccc3)c3ccc4c(c3)[C@]3(CCc5ccc(N(c6ccccc6)c6ccccc6)cc53)CC4)ccc1C2(C)C ; CC(C)c1c(N(c2ccccc2)c2ccc3c(c2)[C@]2(CCc4ccc(N(c5ccccc5)c5ccccc5)cc42)CC3)ccc2c1-c1ccccc1C2(C)C]

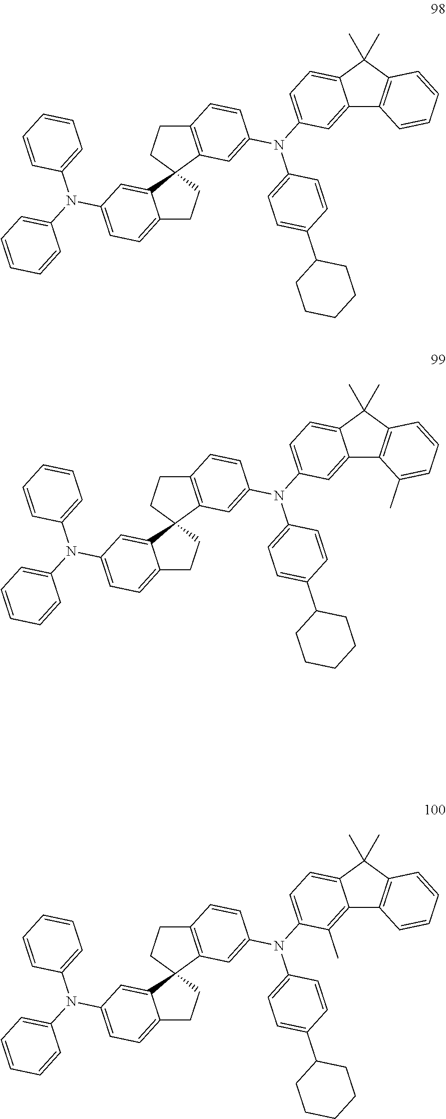 [SMILES: CC1(C)c2ccccc2-c2cc(N(c3ccc(C4CCCCC4)cc3)c3ccc4c(c3)[C@]3(CCc5ccc(N(c6ccccc6)c6ccccc6)cc53)CC4)ccc21 ; Cc1cccc2c1-c1cc(N(c3ccc(C4CCCCC4)cc3)c3ccc4c(c3)[C@]3(CCc5ccc(N(c6ccccc6)c6ccccc6)cc53)CC4)ccc1C2(C)C ; Cc1c(N(c2ccc(C3CCCCC3)cc2)c2ccc3c(c2)[C@]2(CCc4ccc(N(c5ccccc5)c5ccccc5)cc42)CC3)ccc2c1-c1ccccc1C2(C)C]

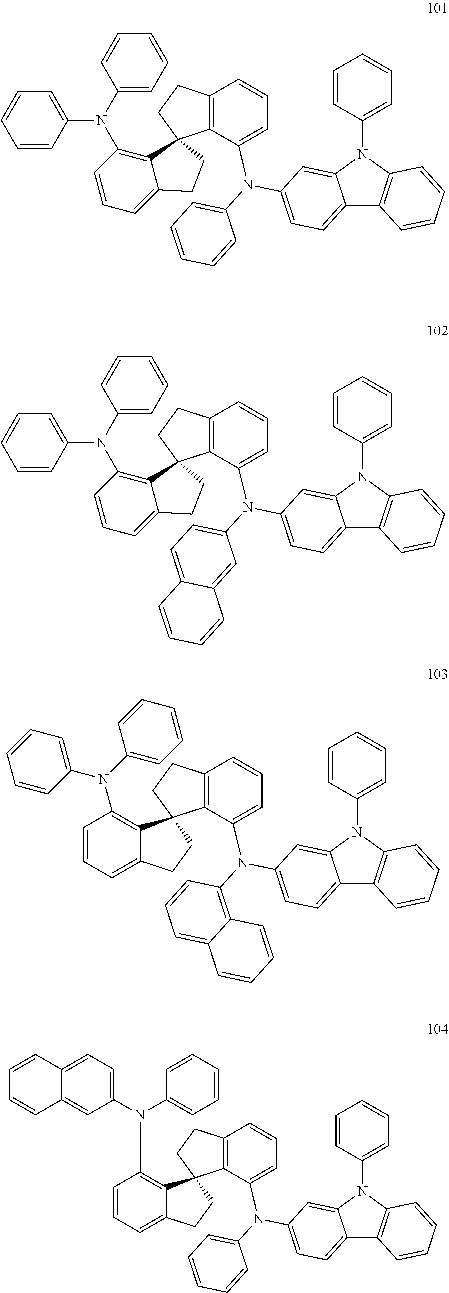 [SMILES: c1ccc(N(c2ccccc2)c2cccc3c2[C@@]2(CC3)CCc3cccc(N(c4ccccc4)c4ccc5c6ccccc6n(-c6ccccc6)c5c4)c32)cc1 ; c1ccc(N(c2ccccc2)c2cccc3c2[C@@]2(CC3)CCc3cccc(N(c4ccc5ccccc5c4)c4ccc5c6ccccc6n(-c6ccccc6)c5c4)c32)cc1 ; c1ccc(N(c2ccccc2)c2cccc3c2[C@@]2(CC3)CCc3cccc(N(c4ccc5c6ccccc6n(-c6ccccc6)c5c4)c4cccc5ccccc45)c32)cc1 ; c1ccc(N(c2ccc3ccccc3c2)c2cccc3c2[C@@]2(CC3)CCc3cccc(N(c4ccccc4)c4ccc5c6ccccc6n(-c6ccccc6)c5c4)c32)cc1]

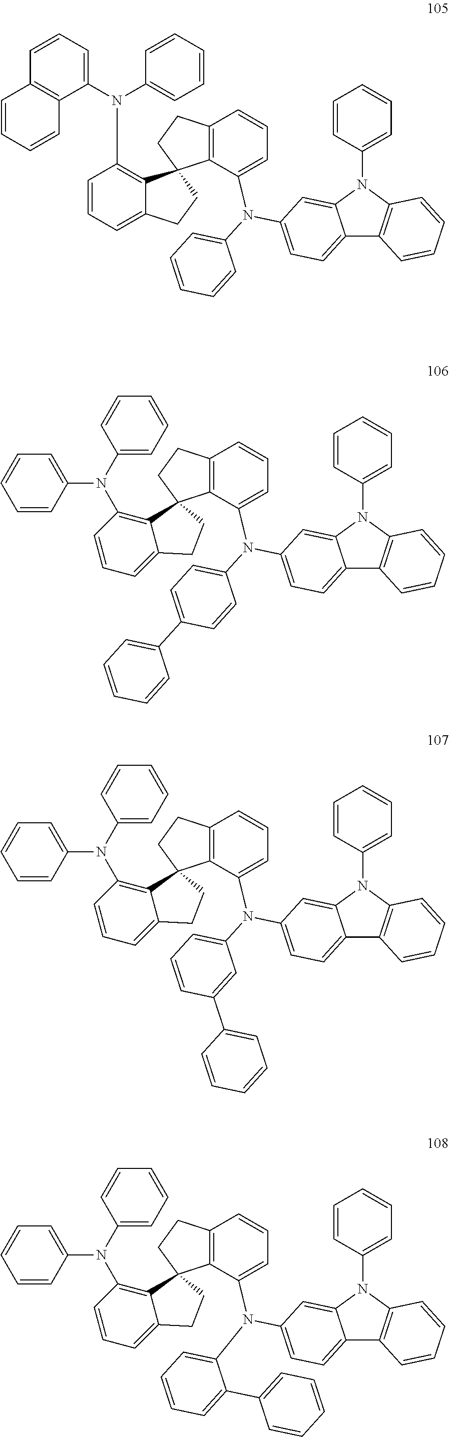 [SMILES: c1ccc(N(c2ccc3c4ccccc4n(-c4ccccc4)c3c2)c2cccc3c2[C@@]2(CC3)CCc3cccc(N(c4ccccc4)c4cccc5ccccc45)c32)cc1 ; c1ccc(-c2ccc(N(c3ccc4c5ccccc5n(-c5ccccc5)c4c3)c3cccc4c3[C@]3(CCc5cccc(N(c6ccccc6)c6ccccc6)c53)CC4)cc2)cc1 ; c1ccc(-c2cccc(N(c3ccc4c5ccccc5n(-c5ccccc5)c4c3)c3cccc4c3[C@]3(CCc5cccc(N(c6ccccc6)c6ccccc6)c53)CC4)c2)cc1 ; c1ccc(-c2ccccc2N(c2ccc3c4ccccc4n(-c4ccccc4)c3c2)c2cccc3c2[C@]2(CCc4cccc(N(c5ccccc5)c5ccccc5)c42)CC3)cc1]

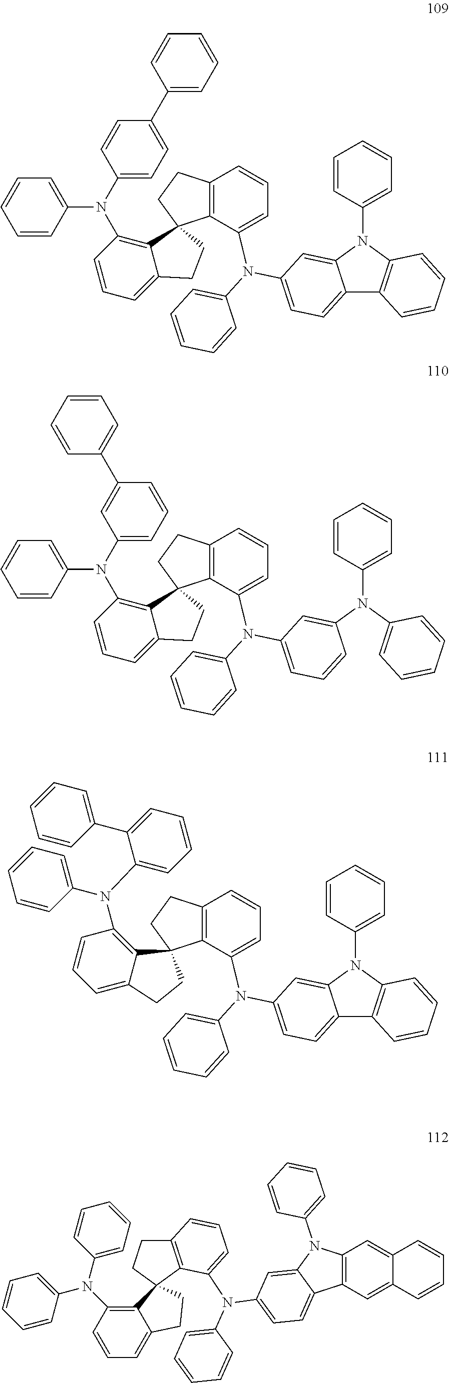 [SMILES: c1ccc(-c2ccc(N(c3ccccc3)c3cccc4c3[C@@]3(CC4)CCc4cccc(N(c5ccccc5)c5ccc6c7ccccc7n(-c7ccccc7)c6c5)c43)cc2)cc1 ; c1ccc(-c2cccc(N(c3ccccc3)c3cccc4c3[C@@]3(CC4)CCc4cccc(N(c5ccccc5)c5cccc(N(c6ccccc6)c6ccccc6)c5)c43)c2)cc1 ; c1ccc(-c2ccccc2N(c2ccccc2)c2cccc3c2[C@]2(CCc4cccc(N(c5ccccc5)c5ccc6c7ccccc7n(-c7ccccc7)c6c5)c42)CC3)cc1 ; c1ccc(N(c2ccccc2)c2cccc3c2[C@@]2(CC3)CCc3cccc(N(c4ccccc4)c4ccc5c6cc7ccccc7cc6n(-c6ccccc6)c5c4)c32)cc1]

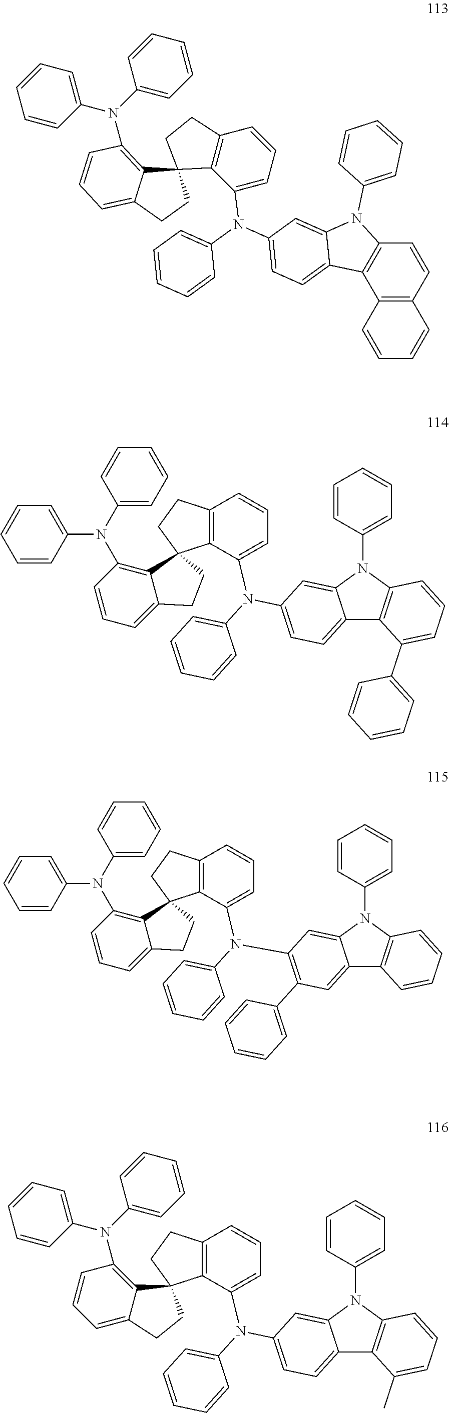 [SMILES: c1ccc(N(c2ccccc2)c2cccc3c2[C@@]2(CC3)CCc3cccc(N(c4ccccc4)c4ccc5c6c7ccccc7ccc6n(-c6ccccc6)c5c4)c32)cc1 ; c1ccc(-c2cccc3c2c2ccc(N(c4ccccc4)c4cccc5c4[C@]4(CCc6cccc(N(c7ccccc7)c7ccccc7)c64)CC5)cc2n3-c2ccccc2)cc1 ; c1ccc(-c2cc3c4ccccc4n(-c4ccccc4)c3cc2N(c2ccccc2)c2cccc3c2[C@]2(CCc4cccc(N(c5ccccc5)c5ccccc5)c42)CC3)cc1 ; Cc1cccc2c1c1ccc(N(c3ccccc3)c3cccc4c3[C@]3(CCc5cccc(N(c6ccccc6)c6ccccc6)c53)CC4)cc1n2-c1ccccc1]

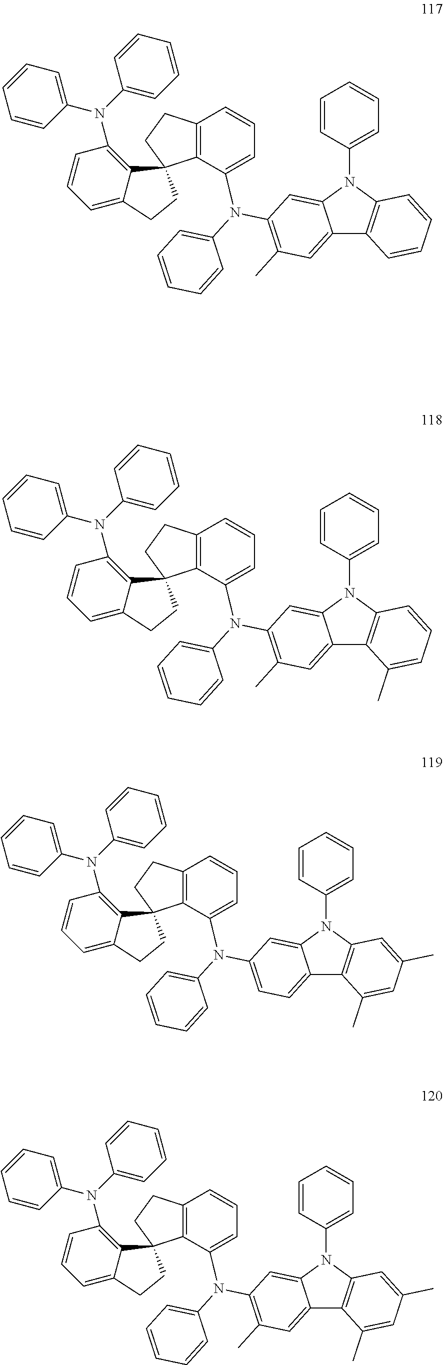 [SMILES: Cc1cc2c3ccccc3n(-c3ccccc3)c2cc1N(c1ccccc1)c1cccc2c1[C@]1(CCc3cccc(N(c4ccccc4)c4ccccc4)c31)CC2 ; Cc1cc2c3c(C)cccc3n(-c3ccccc3)c2cc1N(c1ccccc1)c1cccc2c1[C@]1(CCc3cccc(N(c4ccccc4)c4ccccc4)c31)CC2 ; Cc1cc(C)c2c3ccc(N(c4ccccc4)c4cccc5c4[C@]4(CCc6cccc(N(c7ccccc7)c7ccccc7)c64)CC5)cc3n(-c3ccccc3)c2c1 ; Cc1cc(C)c2c3cc(C)c(N(c4ccccc4)c4cccc5c4[C@]4(CCc6cccc(N(c7ccccc7)c7ccccc7)c64)CC5)cc3n(-c3ccccc3)c2c1]

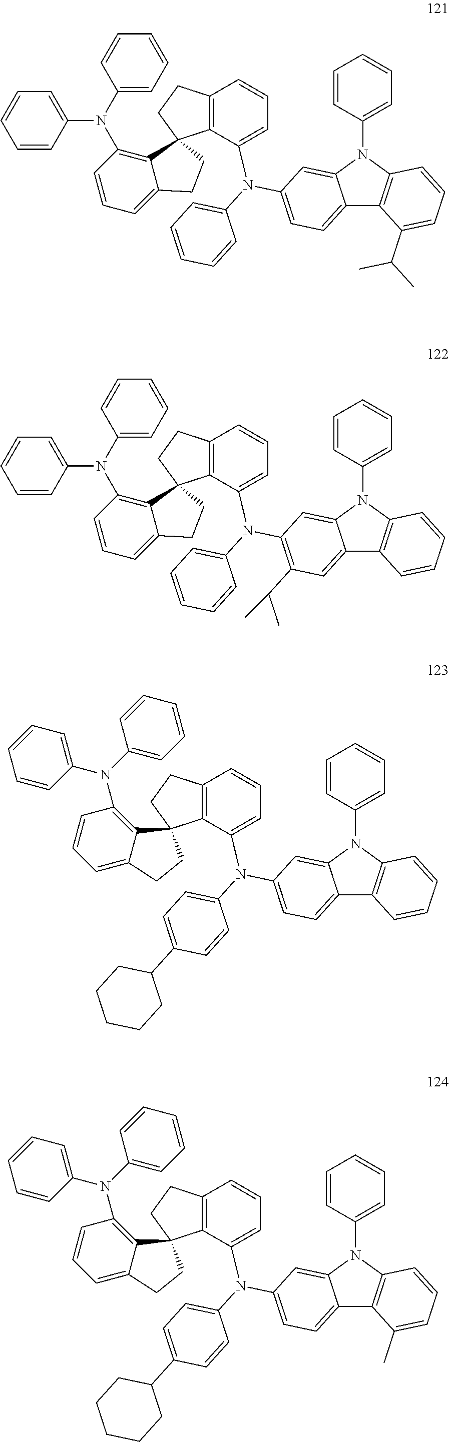 [SMILES: CC(C)c1cccc2c1c1ccc(N(c3ccccc3)c3cccc4c3[C@]3(CCc5cccc(N(c6ccccc6)c6ccccc6)c53)CC4)cc1n2-c1ccccc1 ; CC(C)c1cc2c3ccccc3n(-c3ccccc3)c2cc1N(c1ccccc1)c1cccc2c1[C@]1(CCc3cccc(N(c4ccccc4)c4ccccc4)c31)CC2 ; c1ccc(N(c2ccccc2)c2cccc3c2[C@@]2(CC3)CCc3cccc(N(c4ccc(C5CCCCC5)cc4)c4ccc5c6ccccc6n(-c6ccccc6)c5c4)c32)cc1 ; Cc1cccc2c1c1ccc(N(c3ccc(C4CCCCC4)cc3)c3cccc4c3[C@]3(CCc5cccc(N(c6ccccc6)c6ccccc6)c53)CC4)cc1n2-c1ccccc1]

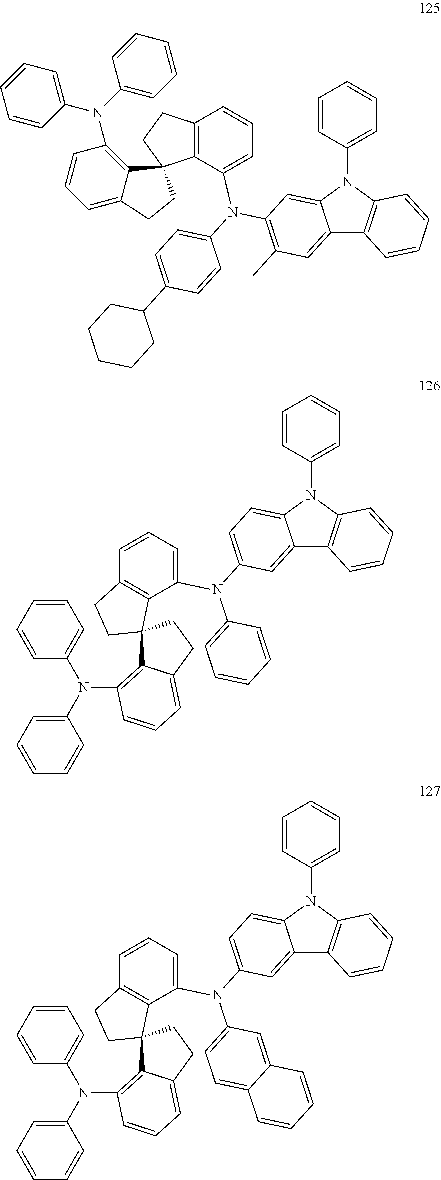 [SMILES: Cc1cc2c3ccccc3n(-c3ccccc3)c2cc1N(c1ccc(C2CCCCC2)cc1)c1cccc2c1[C@]1(CCc3cccc(N(c4ccccc4)c4ccccc4)c31)CC2 ; c1ccc(N(c2ccccc2)c2cccc3c2[C@@]2(CC3)CCc3cccc(N(c4ccccc4)c4ccc5c(c4)c4ccccc4n5-c4ccccc4)c32)cc1 ; c1ccc(N(c2ccccc2)c2cccc3c2[C@@]2(CC3)CCc3cccc(N(c4ccc5ccccc5c4)c4ccc5c(c4)c4ccccc4n5-c4ccccc4)c32)cc1]

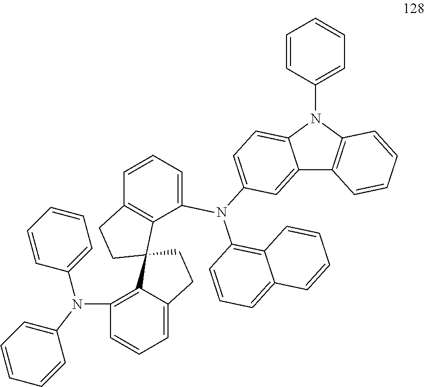 [SMILES: c1ccc(N(c2ccccc2)c2cccc3c2[C@@]2(CC3)CCc3cccc(N(c4ccc5c(c4)c4ccccc4n5-c4ccccc4)c4cccc5ccccc45)c32)cc1]

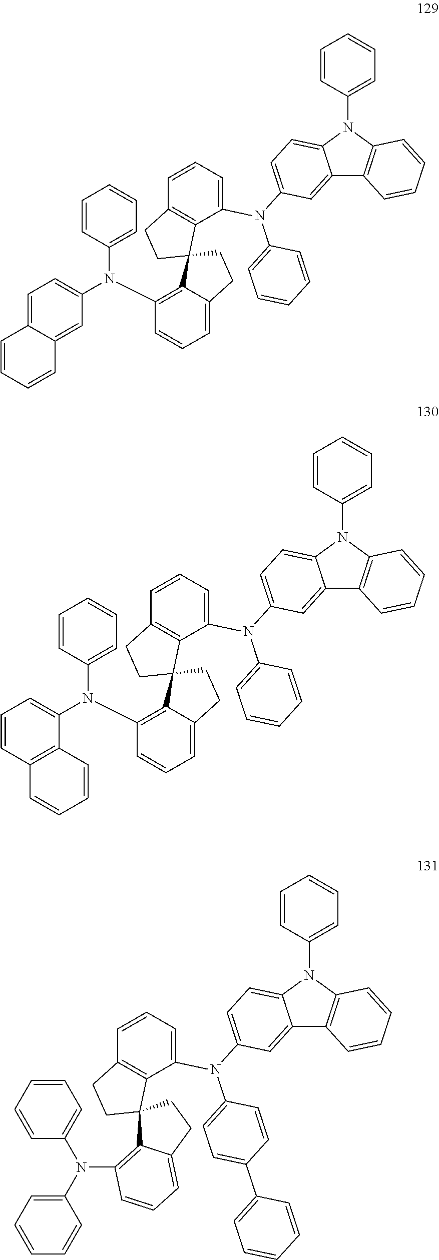 [SMILES: c1ccc(N(c2ccc3ccccc3c2)c2cccc3c2[C@@]2(CC3)CCc3cccc(N(c4ccccc4)c4ccc5c(c4)c4ccccc4n5-c4ccccc4)c32)cc1 ; c1ccc(N(c2ccc3c(c2)c2ccccc2n3-c2ccccc2)c2cccc3c2[C@@]2(CC3)CCc3cccc(N(c4ccccc4)c4cccc5ccccc45)c32)cc1 ; c1ccc(-c2ccc(N(c3ccc4c(c3)c3ccccc3n4-c3ccccc3)c3cccc4c3[C@]3(CCc5cccc(N(c6ccccc6)c6ccccc6)c53)CC4)cc2)cc1]

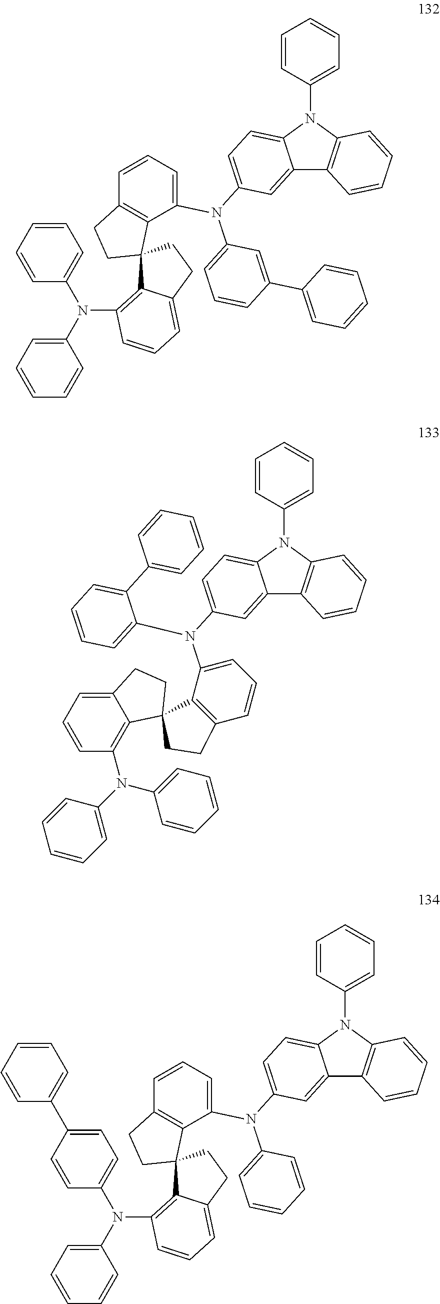 [SMILES: c1ccc(-c2cccc(N(c3ccc4c(c3)c3ccccc3n4-c3ccccc3)c3cccc4c3[C@]3(CCc5cccc(N(c6ccccc6)c6ccccc6)c53)CC4)c2)cc1 ; c1ccc(-c2ccccc2N(c2ccc3c(c2)c2ccccc2n3-c2ccccc2)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6c(c5)c5ccccc5n6-c5ccccc5)c43)cc2)cc1]

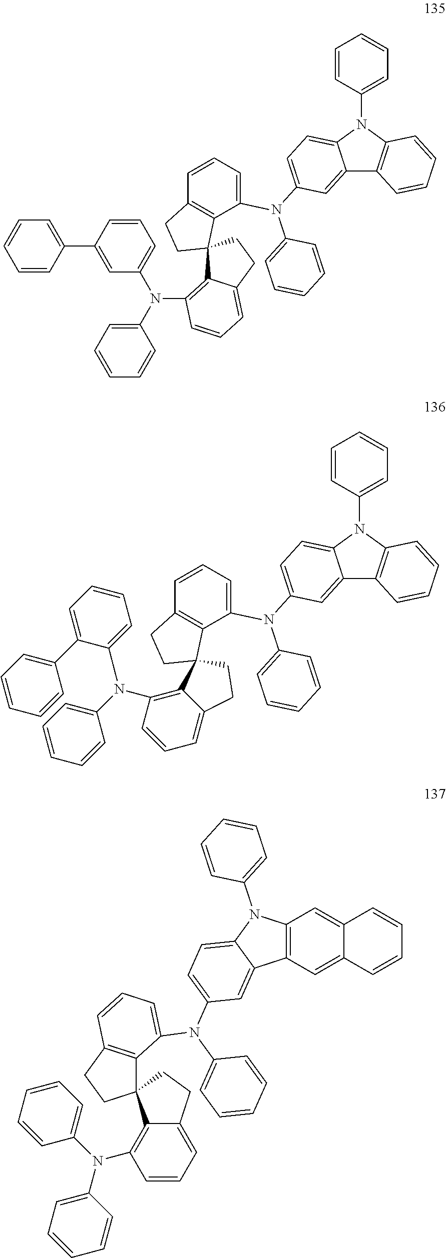

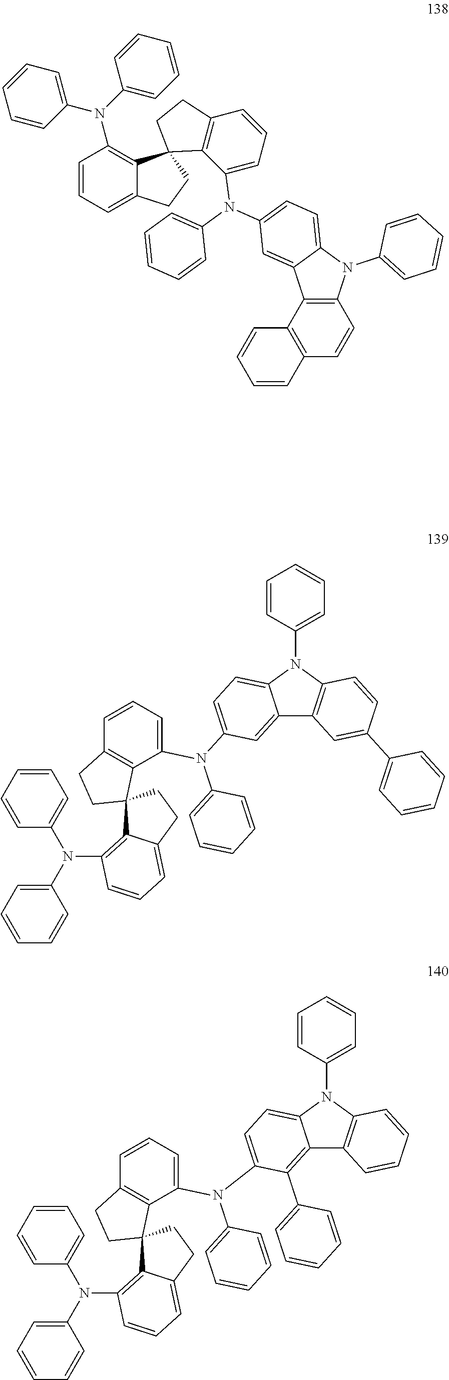 [SMILES: c1ccc(N(c2ccccc2)c2cccc3c2[C@@]2(CC3)CCc3cccc(N(c4ccccc4)c4ccc5c(c4)c4c6ccccc6ccc4n5-c4ccccc4)c32)cc1 ; c1ccc(-c2ccc3c(c2)c2cc(N(c4ccccc4)c4cccc5c4[C@]4(CCc6cccc(N(c7ccccc7)c7ccccc7)c64)CC5)ccc2n3-c2ccccc2)cc1 ; c1ccc(-c2c(N(c3ccccc3)c3cccc4c3[C@]3(CCc5cccc(N(c6ccccc6)c6ccccc6)c53)CC4)ccc3c2c2ccccc2n3-c2ccccc2)cc1]

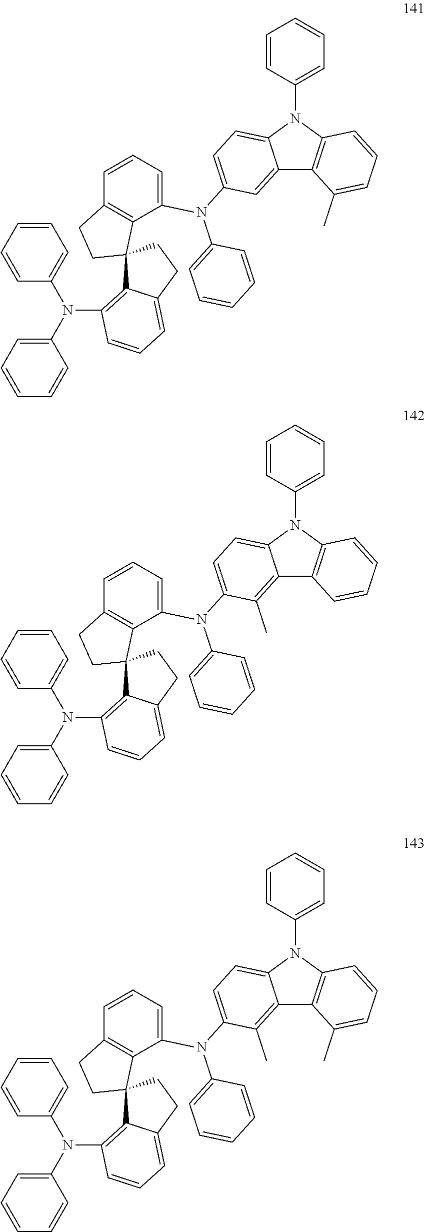 [SMILES: Cc1cccc2c1c1cc(N(c3ccccc3)c3cccc4c3[C@]3(CCc5cccc(N(c6ccccc6)c6ccccc6)c53)CC4)ccc1n2-c1ccccc1 ; Cc1c(N(c2ccccc2)c2cccc3c2[C@]2(CCc4cccc(N(c5ccccc5)c5ccccc5)c42)CC3)ccc2c1c1ccccc1n2-c1ccccc1 ; Cc1cccc2c1c1c(C)c(N(c3ccccc3)c3cccc4c3[C@]3(CCc5cccc(N(c6ccccc6)c6ccccc6)c53)CC4)ccc1n2-c1ccccc1]

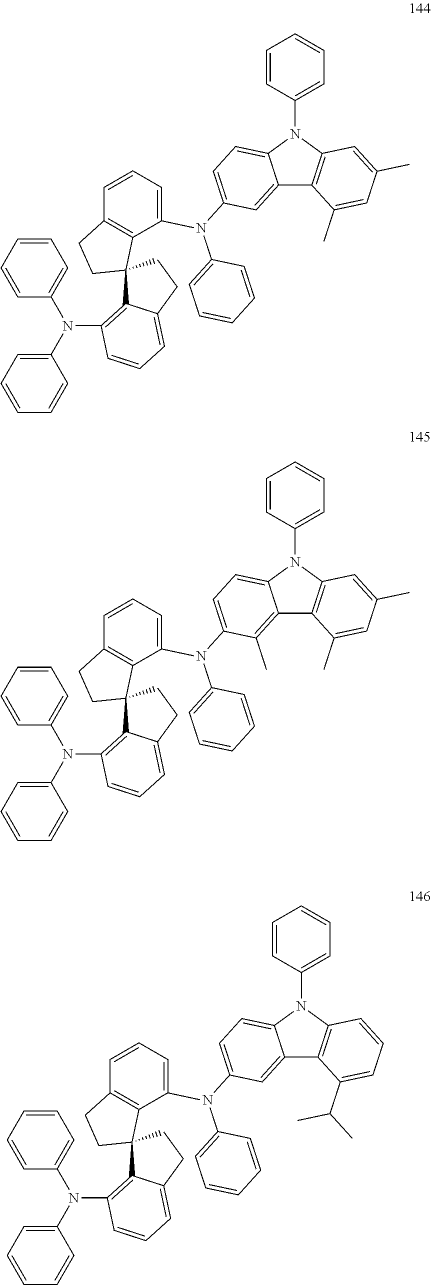 [SMILES: Cc1cc(C)c2c3cc(N(c4ccccc4)c4cccc5c4[C@]4(CCc6cccc(N(c7ccccc7)c7ccccc7)c64)CC5)ccc3n(-c3ccccc3)c2c1 ; Cc1cc(C)c2c3c(C)c(N(c4ccccc4)c4cccc5c4[C@]4(CCc6cccc(N(c7ccccc7)c7ccccc7)c64)CC5)ccc3n(-c3ccccc3)c2c1 ; CC(C)c1cccc2c1c1cc(N(c3ccccc3)c3cccc4c3[C@]3(CCc5cccc(N(c6ccccc6)c6ccccc6)c53)CC4)ccc1n2-c1ccccc1]

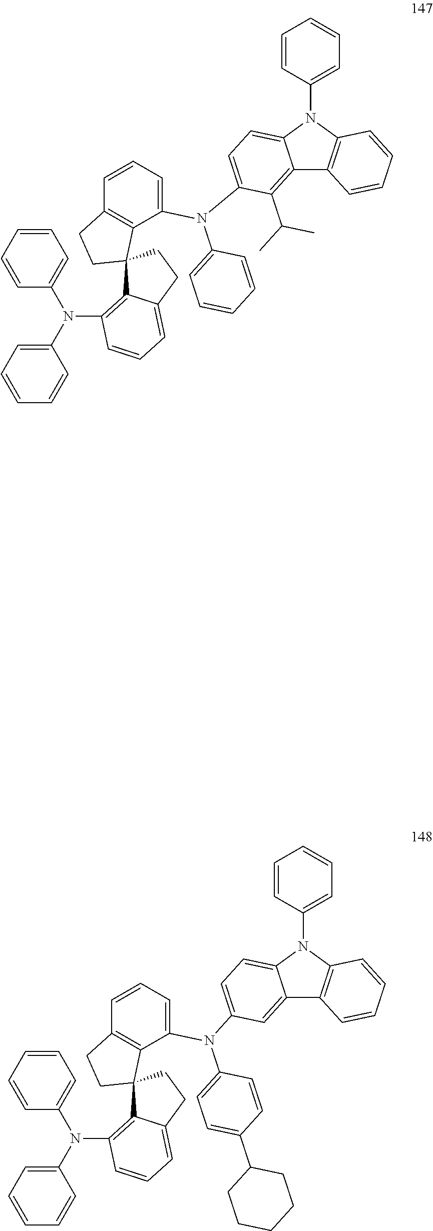 [SMILES: CC(C)c1c(N(c2ccccc2)c2cccc3c2[C@]2(CCc4cccc(N(c5ccccc5)c5ccccc5)c42)CC3)ccc2c1c1ccccc1n2-c1ccccc1 ; c1ccc(N(c2ccccc2)c2cccc3c2[C@@]2(CC3)CCc3cccc(N(c4ccc(C5CCCCC5)cc4)c4ccc5c(c4)c4ccccc4n5-c4ccccc4)c32)cc1]

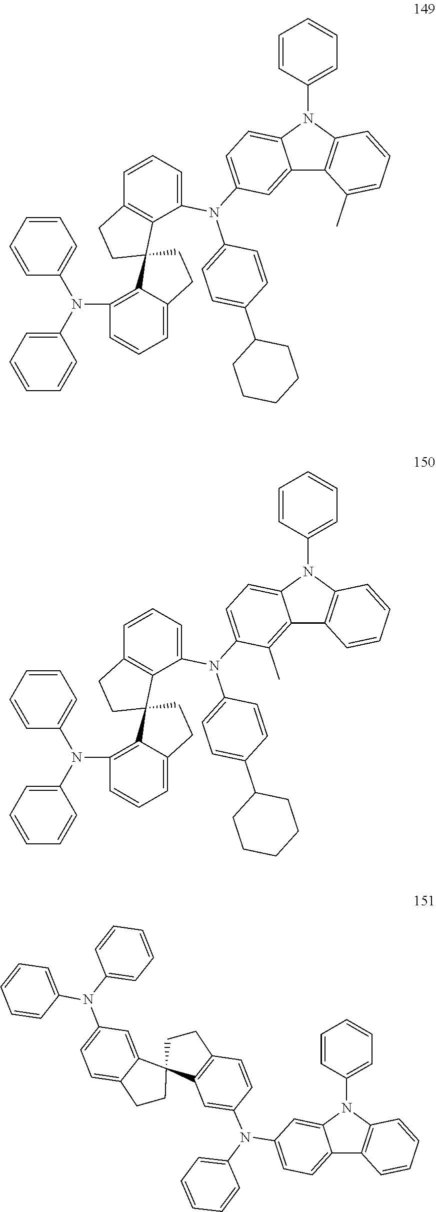 [SMILES: Cc1cccc2c1c1cc(N(c3ccc(C4CCCCC4)cc3)c3cccc4c3[C@]3(CCc5cccc(N(c6ccccc6)c6ccccc6)c53)CC4)ccc1n2-c1ccccc1 ; Cc1c(N(c2ccc(C3CCCCC3)cc2)c2cccc3c2[C@]2(CCc4cccc(N(c5ccccc5)c5ccccc5)c42)CC3)ccc2c1c1ccccc1n2-c1ccccc1 ; c1ccc(N(c2ccccc2)c2ccc3c(c2)[C@@]2(CC3)CCc3ccc(N(c4ccccc4)c4ccc5c6ccccc6n(-c6ccccc6)c5c4)cc32)cc1]

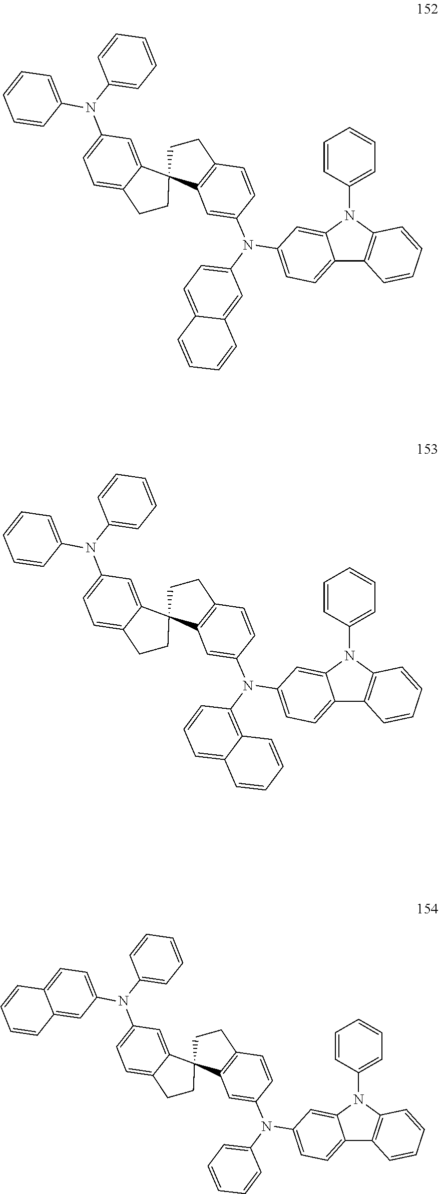 [SMILES: c1ccc(N(c2ccccc2)c2ccc3c(c2)[C@@]2(CC3)CCc3ccc(N(c4ccc5ccccc5c4)c4ccc5c6ccccc6n(-c6ccccc6)c5c4)cc32)cc1 ; c1ccc(N(c2ccccc2)c2ccc3c(c2)[C@@]2(CC3)CCc3ccc(N(c4ccc5c6ccccc6n(-c6ccccc6)c5c4)c4cccc5ccccc45)cc32)cc1 ; c1ccc(N(c2ccc3c(c2)[C@@]2(CC3)CCc3ccc(N(c4ccccc4)c4ccc5c6ccccc6n(-c6ccccc6)c5c4)cc32)c2ccc3ccccc3c2)cc1]

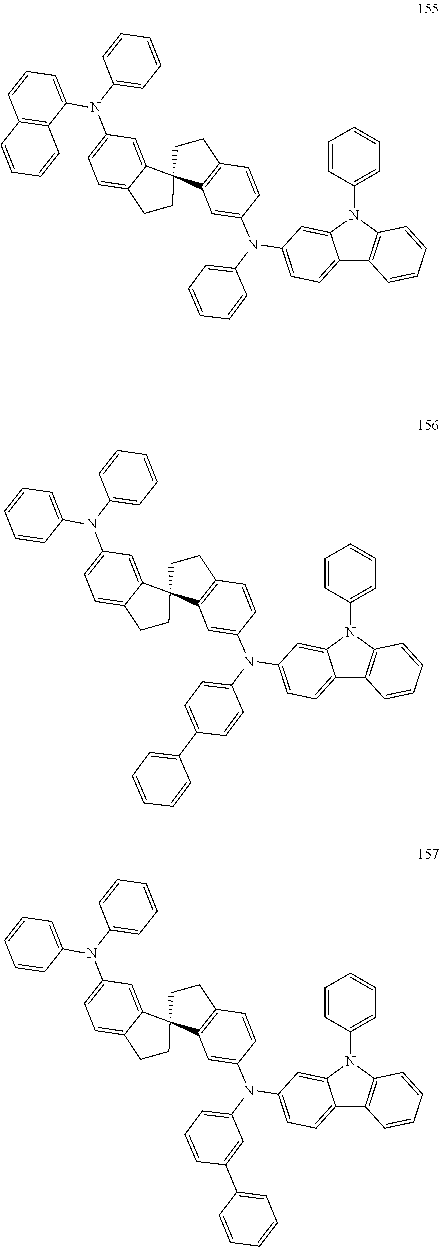 [SMILES: c1ccc(N(c2ccc3c(c2)[C@@]2(CC3)CCc3ccc(N(c4ccccc4)c4cccc5ccccc45)cc32)c2ccc3c4ccccc4n(-c4ccccc4)c3c2)cc1 ; c1ccc(-c2ccc(N(c3ccc4c(c3)[C@]3(CCc5ccc(N(c6ccccc6)c6ccccc6)cc53)CC4)c3ccc4c5ccccc5n(-c5ccccc5)c4c3)cc2)cc1 ; c1ccc(-c2cccc(N(c3ccc4c(c3)[C@]3(CCc5ccc(N(c6ccccc6)c6ccccc6)cc53)CC4)c3ccc4c5ccccc5n(-c5ccccc5)c4c3)c2)cc1]

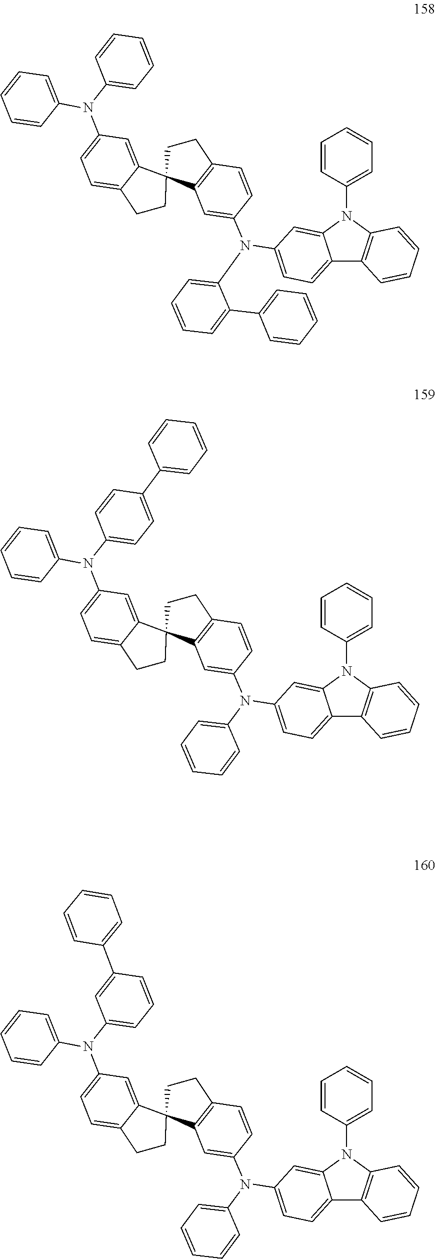 [SMILES: c1ccc(-c2ccccc2N(c2ccc3c(c2)[C@]2(CCc4ccc(N(c5ccccc5)c5ccccc5)cc42)CC3)c2ccc3c4ccccc4n(-c4ccccc4)c3c2)cc1 ; c1ccc(-c2ccc(N(c3ccccc3)c3ccc4c(c3)[C@@]3(CC4)CCc4ccc(N(c5ccccc5)c5ccc6c7ccccc7n(-c7ccccc7)c6c5)cc43)cc2)cc1 ; c1ccc(-c2cccc(N(c3ccccc3)c3ccc4c(c3)[C@@]3(CC4)CCc4ccc(N(c5ccccc5)c5ccc6c7ccccc7n(-c7ccccc7)c6c5)cc43)c2)cc1]

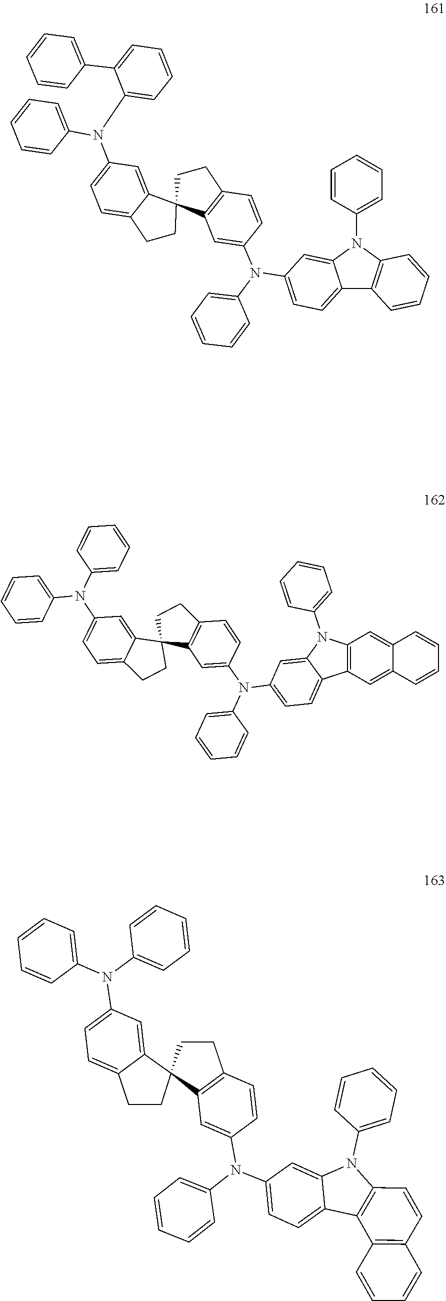 [SMILES: c1ccc(-c2ccccc2N(c2ccccc2)c2ccc3c(c2)[C@]2(CCc4ccc(N(c5ccccc5)c5ccc6c7ccccc7n(-c7ccccc7)c6c5)cc42)CC3)cc1 ; c1ccc(N(c2ccccc2)c2ccc3c(c2)[C@@]2(CC3)CCc3ccc(N(c4ccccc4)c4ccc5c6cc7ccccc7cc6n(-c6ccccc6)c5c4)cc32)cc1 ; c1ccc(N(c2ccccc2)c2ccc3c(c2)[C@@]2(CC3)CCc3ccc(N(c4ccccc4)c4ccc5c6c7ccccc7ccc6n(-c6ccccc6)c5c4)cc32)cc1]

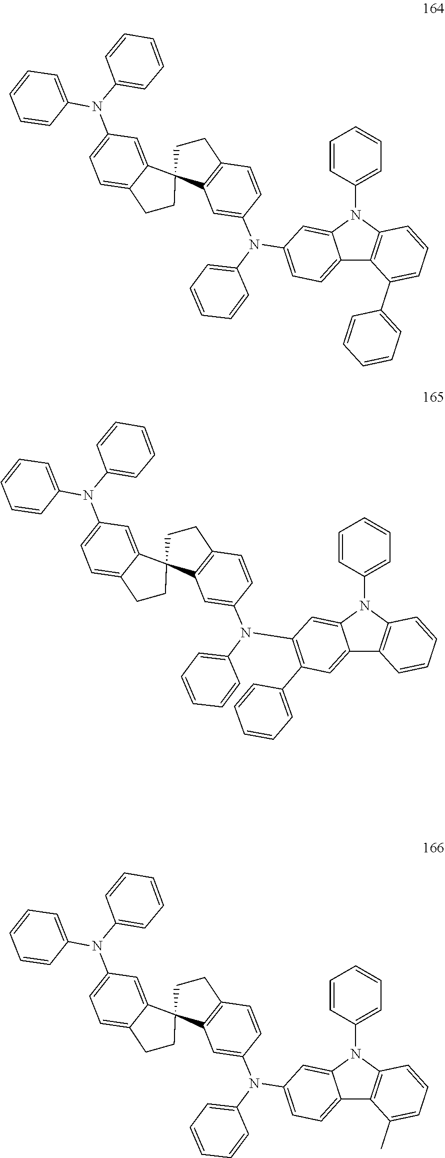 [SMILES: c1ccc(-c2cccc3c2c2ccc(N(c4ccccc4)c4ccc5c(c4)[C@]4(CCc6ccc(N(c7ccccc7)c7ccccc7)cc64)CC5)cc2n3-c2ccccc2)cc1 ; c1ccc(-c2cc3c4ccccc4n(-c4ccccc4)c3cc2N(c2ccccc2)c2ccc3c(c2)[C@]2(CCc4ccc(N(c5ccccc5)c5ccccc5)cc42)CC3)cc1 ; Cc1cccc2c1c1ccc(N(c3ccccc3)c3ccc4c(c3)[C@]3(CCc5ccc(N(c6ccccc6)c6ccccc6)cc53)CC4)cc1n2-c1ccccc1]

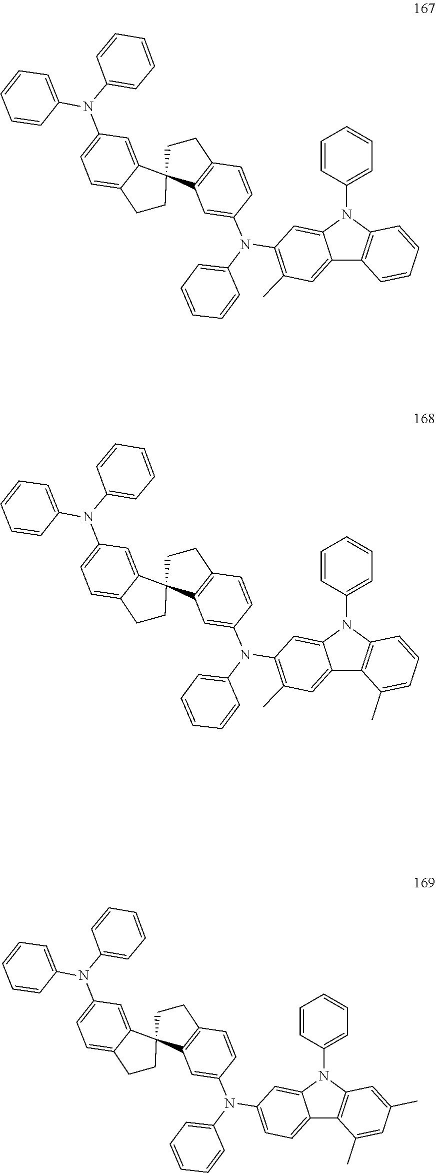 [SMILES: Cc1cc2c3ccccc3n(-c3ccccc3)c2cc1N(c1ccccc1)c1ccc2c(c1)[C@]1(CCc3ccc(N(c4ccccc4)c4ccccc4)cc31)CC2 ; Cc1cc2c3c(C)cccc3n(-c3ccccc3)c2cc1N(c1ccccc1)c1ccc2c(c1)[C@]1(CCc3ccc(N(c4ccccc4)c4ccccc4)cc31)CC2 ; Cc1cc(C)c2c3ccc(N(c4ccccc4)c4ccc5c(c4)[C@]4(CCc6ccc(N(c7ccccc7)c7ccccc7)cc64)CC5)cc3n(-c3ccccc3)c2c1]

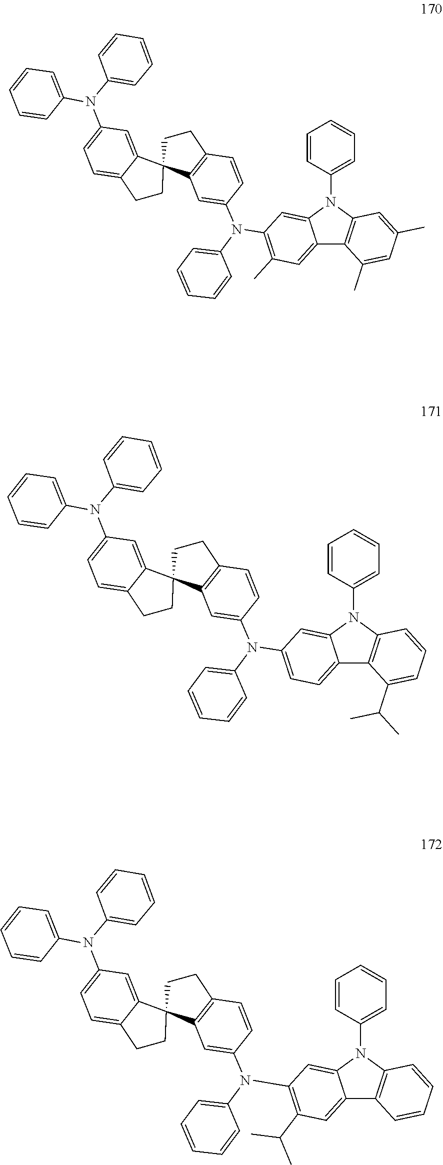 [SMILES: Cc1cc(C)c2c3cc(C)c(N(c4ccccc4)c4ccc5c(c4)[C@]4(CCc6ccc(N(c7ccccc7)c7ccccc7)cc64)CC5)cc3n(-c3ccccc3)c2c1 ; CC(C)c1cccc2c1c1ccc(N(c3ccccc3)c3ccc4c(c3)[C@]3(CCc5ccc(N(c6ccccc6)c6ccccc6)cc53)CC4)cc1n2-c1ccccc1 ; CC(C)c1cc2c3ccccc3n(-c3ccccc3)c2cc1N(c1ccccc1)c1ccc2c(c1)[C@]1(CCc3ccc(N(c4ccccc4)c4ccccc4)cc31)CC2]

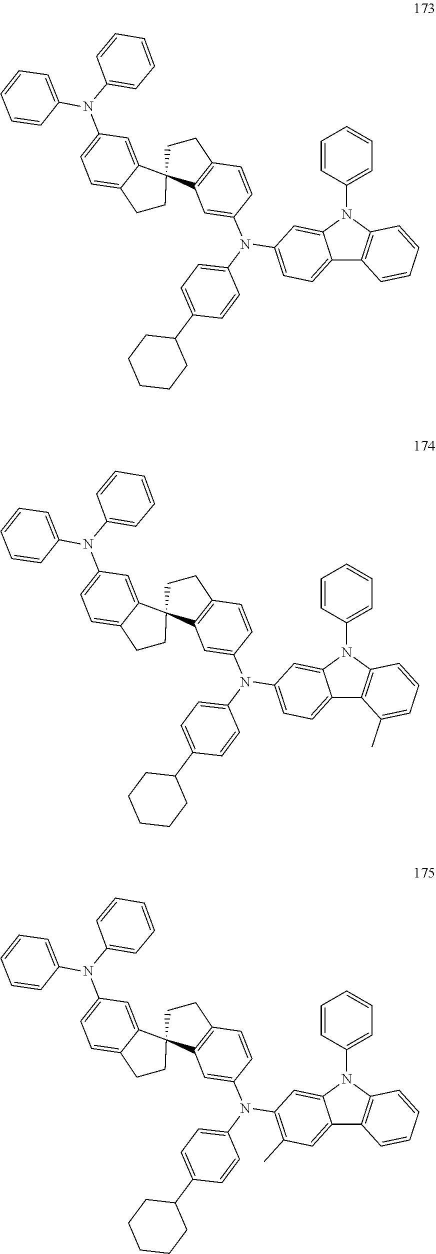 [SMILES: c1ccc(N(c2ccccc2)c2ccc3c(c2)[C@@]2(CC3)CCc3ccc(N(c4ccc(C5CCCCC5)cc4)c4ccc5c6ccccc6n(-c6ccccc6)c5c4)cc32)cc1 ; Cc1cccc2c1c1ccc(N(c3ccc(C4CCCCC4)cc3)c3ccc4c(c3)[C@]3(CCc5ccc(N(c6ccccc6)c6ccccc6)cc53)CC4)cc1n2-c1ccccc1 ; Cc1cc2c3ccccc3n(-c3ccccc3)c2cc1N(c1ccc(C2CCCCC2)cc1)c1ccc2c(c1)[C@]1(CCc3ccc(N(c4ccccc4)c4ccccc4)cc31)CC2]

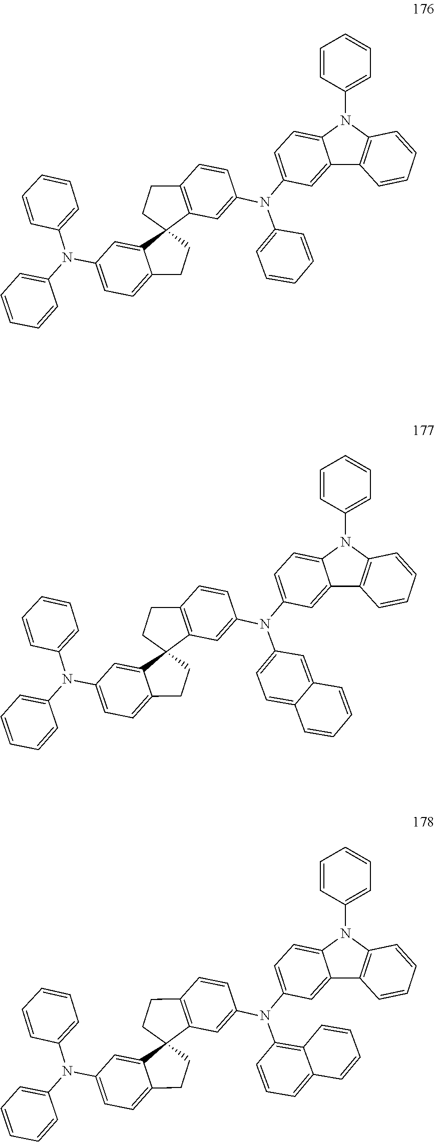 [SMILES: c1ccc(N(c2ccccc2)c2ccc3c(c2)[C@@]2(CC3)CCc3ccc(N(c4ccccc4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5ccccc5c4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5c(c4)c4ccccc4n5-c4ccccc4)c4cccc5ccccc45)cc32)cc1]

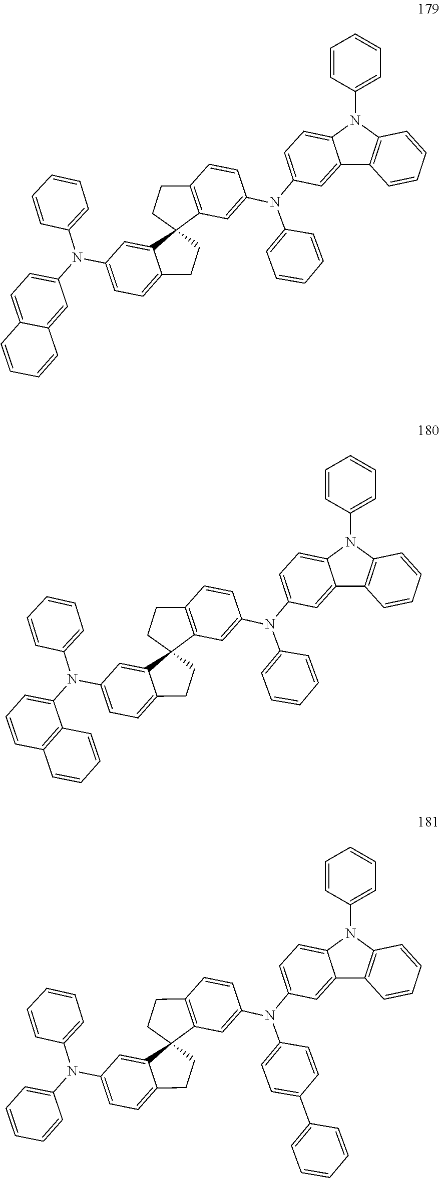 [SMILES: c1ccc(N(c2ccc3c(c2)[C@@]2(CC3)CCc3ccc(N(c4ccccc4)c4ccc5c(c4)c4ccccc4n5-c4ccccc4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)c2ccccc2n3-c2ccccc2)cc1 ; c1ccc(-c2ccc(N(c3ccc4c(c3)[C@]3(CCc5ccc(N(c6ccccc6)c6ccccc6)cc53)CC4)c3ccc4c(c3)c3ccccc3n4-c3ccccc3)cc2)cc1]

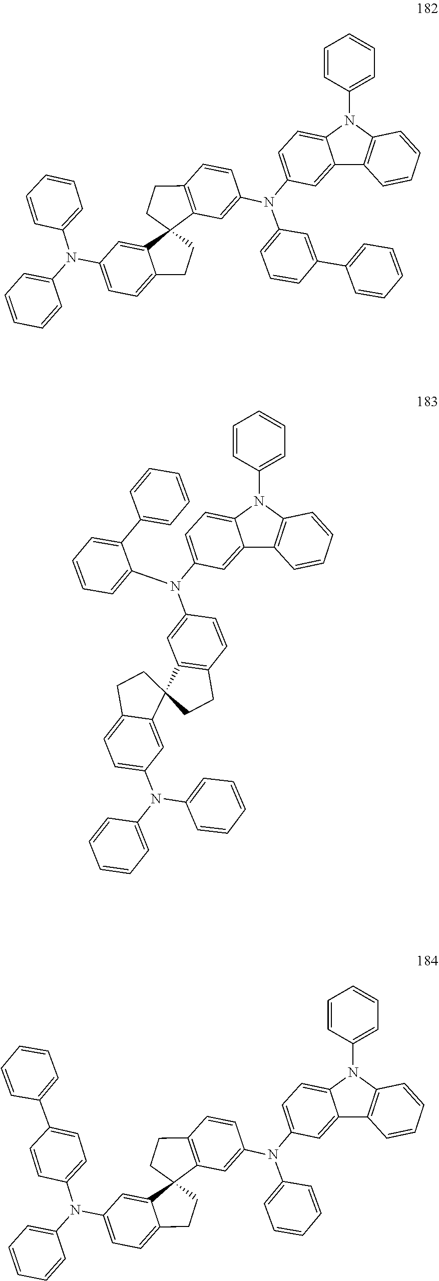 [SMILES: c1ccc(-c2cccc(N(c3ccc4c(c3)[C@]3(CCc5ccc(N(c6ccccc6)c6ccccc6)cc53)CC4)c3ccc4c(c3)c3ccccc3n4-c3ccccc3)c2)cc1 ; c1ccc(-c2ccccc2N(c2ccc3c(c2)[C@]2(CCc4ccc(N(c5ccccc5)c5ccccc5)cc42)CC3)c2ccc3c(c2)c2ccccc2n3-c2ccccc2)cc1 ; c1ccc(-c2ccc(N(c3ccccc3)c3ccc4c(c3)[C@@]3(CC4)CCc4ccc(N(c5ccccc5)c5ccc6c(c5)c5ccccc5n6-c5ccccc5)cc43)cc2)cc1]

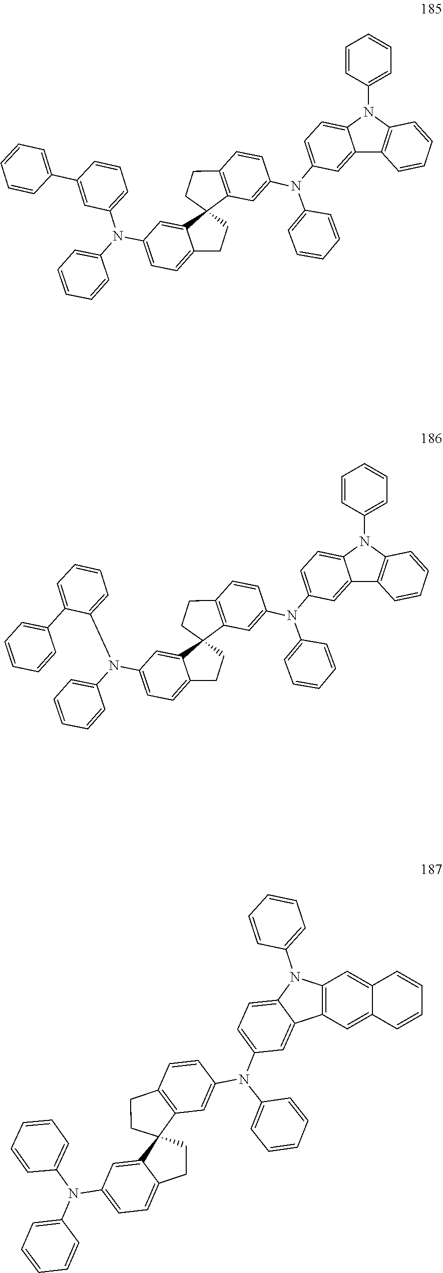 [SMILES: c1ccc(-c2cccc(N(c3ccccc3)c3ccc4c(c3)[C@@]3(CC4)CCc4ccc(N(c5ccccc5)c5ccc6c(c5)c5ccccc5n6-c5ccccc5)cc43)c2)cc1 ; c1ccc(-c2ccccc2N(c2ccccc2)c2ccc3c(c2)[C@]2(CCc4ccc(N(c5ccccc5)c5ccc6c(c5)c5ccccc5n6-c5ccccc5)cc42)CC3)cc1 ; c1ccc(N(c2ccccc2)c2ccc3c(c2)[C@@]2(CC3)CCc3ccc(N(c4ccccc4)c4ccc5c(c4)c4cc6ccccc6cc4n5-c4ccccc4)cc32)cc1]

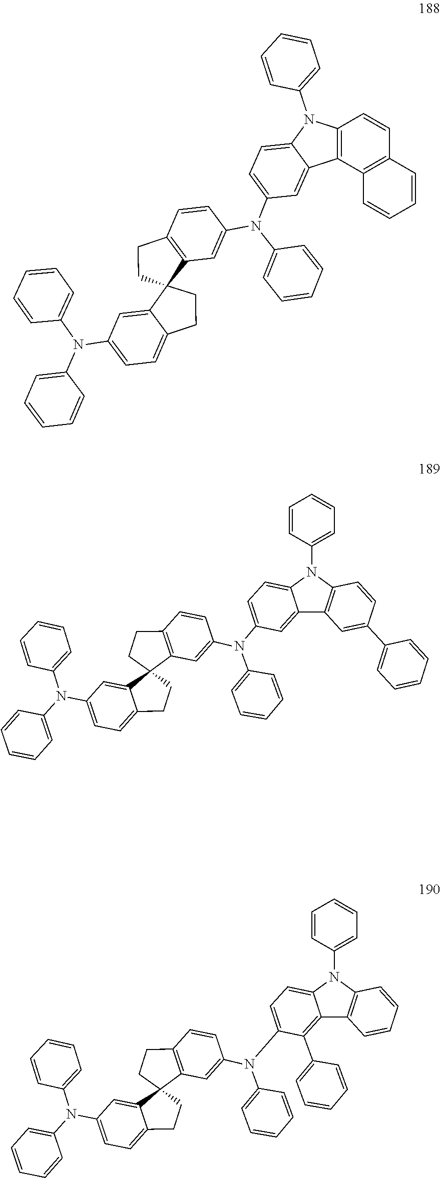 [SMILES: c1ccc(N(c2ccccc2)c2ccc3c(c2)[C@@]2(CC3)CCc3ccc(N(c4ccccc4)c4ccc5c(c4)c4c6ccccc6ccc4n5-c4ccccc4)cc32)cc1 ; c1ccc(-c2ccc3c(c2)c2cc(N(c4ccccc4)c4ccc5c(c4)[C@]4(CCc6ccc(N(c7ccccc7)c7ccccc7)cc64)CC5)ccc2n3-c2ccccc2)cc1 ; c1ccc(-c2c(N(c3ccccc3)c3ccc4c(c3)[C@]3(CCc5ccc(N(c6ccccc6)c6ccccc6)cc53)CC4)ccc3c2c2ccccc2n3-c2ccccc2)cc1]

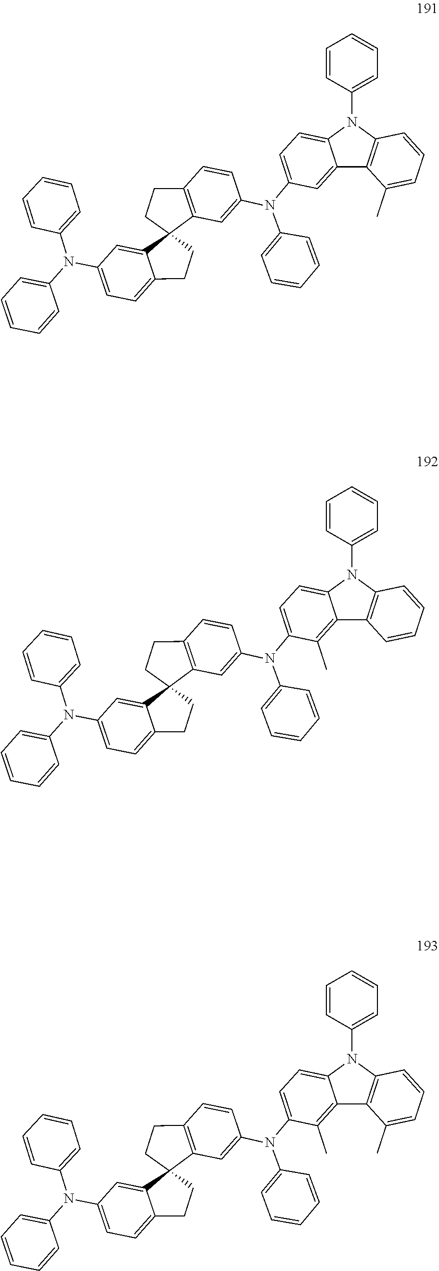 [SMILES: Cc1cccc2c1c1cc(N(c3ccccc3)c3ccc4c(c3)[C@]3(CCc5ccc(N(c6ccccc6)c6ccccc6)cc53)CC4)ccc1n2-c1ccccc1 ; Cc1c(N(c2ccccc2)c2ccc3c(c2)[C@]2(CCc4ccc(N(c5ccccc5)c5ccccc5)cc42)CC3)ccc2c1c1ccccc1n2-c1ccccc1 ; Cc1cccc2c1c1c(C)c(N(c3ccccc3)c3ccc4c(c3)[C@]3(CCc5ccc(N(c6ccccc6)c6ccccc6)cc53)CC4)ccc1n2-c1ccccc1]

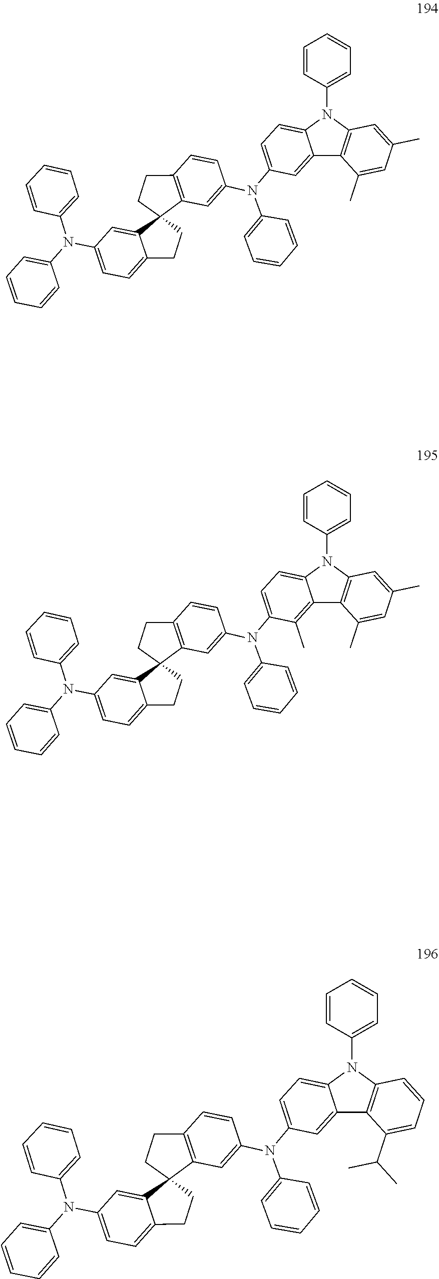 [SMILES: Cc1cc(C)c2c3cc(N(c4ccccc4)c4ccc5c(c4)[C@]4(CCc6ccc(N(c7ccccc7)c7ccccc7)cc64)CC5)ccc3n(-c3ccccc3)c2c1 ; Cc1cc(C)c2c3c(C)c(N(c4ccccc4)c4ccc5c(c4)[C@]4(CCc6ccc(N(c7ccccc7)c7ccccc7)cc64)CC5)ccc3n(-c3ccccc3)c2c1 ; CC(C)c1cccc2c1c1cc(N(c3ccccc3)c3ccc4c(c3)[C@]3(CCc5ccc(N(c6ccccc6)c6ccccc6)cc53)CC4)ccc1n2-c1ccccc1]

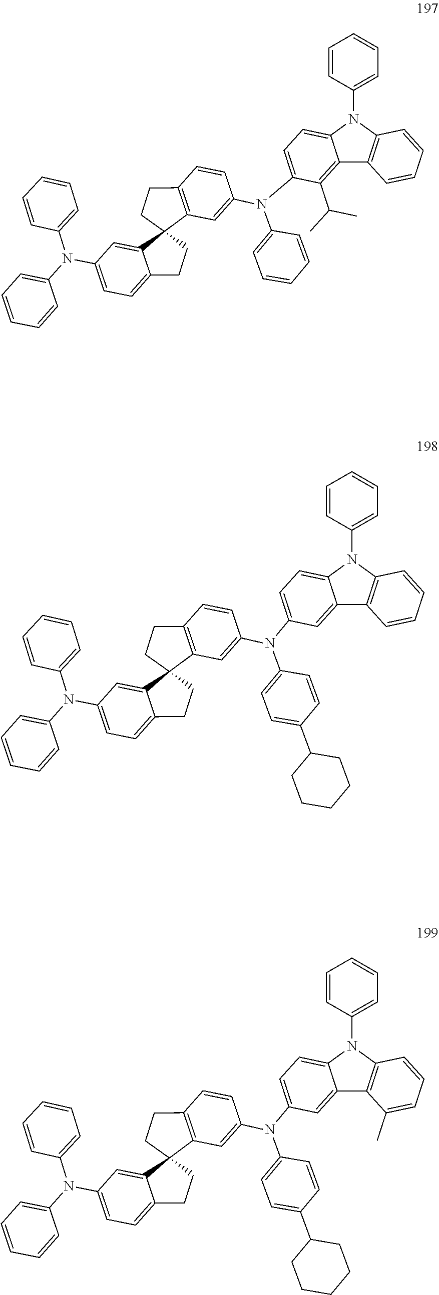 [SMILES: CC(C)c1c(N(c2ccccc2)c2ccc3c(c2)[C@]2(CCc4ccc(N(c5ccccc5)c5ccccc5)cc42)CC3)ccc2c1c1ccccc1n2-c1ccccc1 ; c1ccc(N(c2ccccc2)c2ccc3c(c2)[C@@]2(CC3)CCc3ccc(N(c4ccc(C5CCCCC5)cc4)c4ccc5c(c4)c4ccccc4n5-c4ccccc4)cc32)cc1 ; Cc1cccc2c1c1cc(N(c3ccc(C4CCCCC4)cc3)c3ccc4c(c3)[C@]3(CCc5ccc(N(c6ccccc6)c6ccccc6)cc53)CC4)ccc1n2-c1ccccc1]

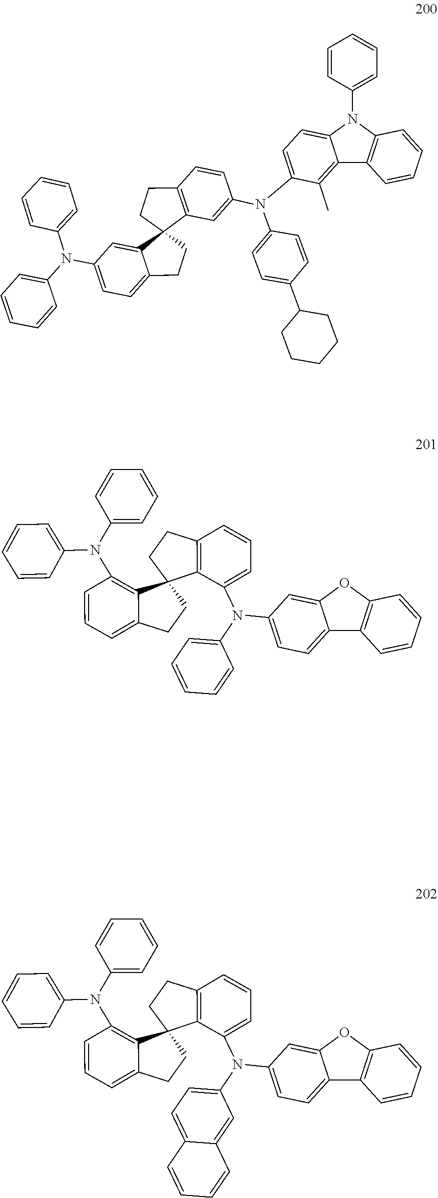 [SMILES: Cc1c(N(c2ccc(C3CCCCC3)cc2)c2ccc3c(c2)[C@]2(CCc4ccc(N(c5ccccc5)c5ccccc5)cc42)CC3)ccc2c1c1ccccc1n2-c1ccccc1 ; c1ccc(N(c2ccccc2)c2cccc3c2[C@@]2(CC3)CCc3cccc(N(c4ccccc4)c4ccc5c(c4)oc4ccccc45)c32)cc1 ; c1ccc(N(c2ccccc2)c2cccc3c2[C@@]2(CC3)CCc3cccc(N(c4ccc5ccccc5c4)c4ccc5c(c4)oc4ccccc45)c32)cc1]

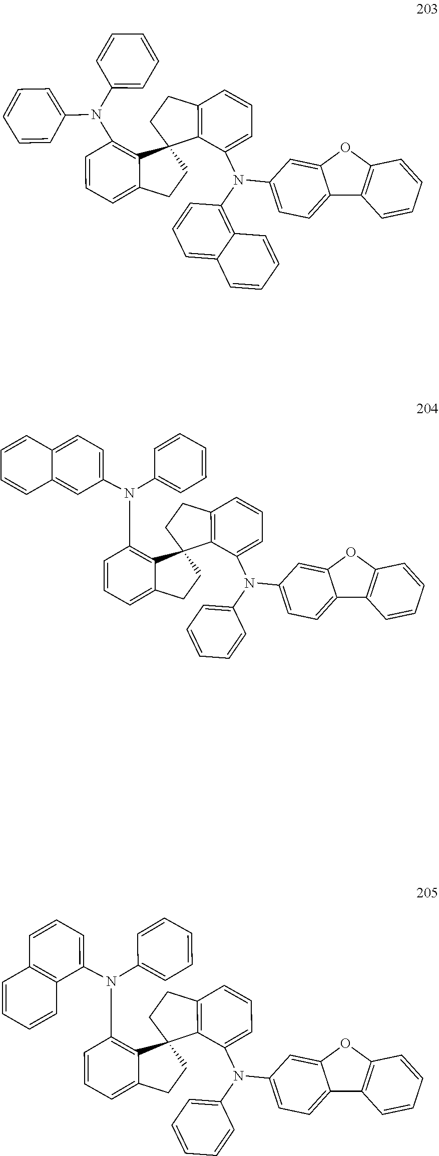 [SMILES: c1ccc(N(c2ccccc2)c2cccc3c2[C@@]2(CC3)CCc3cccc(N(c4ccc5c(c4)oc4ccccc45)c4cccc5ccccc45)c32)cc1 ; c1ccc(N(c2ccc3ccccc3c2)c2cccc3c2[C@@]2(CC3)CCc3cccc(N(c4ccccc4)c4ccc5c(c4)oc4ccccc45)c32)cc1 ; c1ccc(N(c2ccc3c(c2)oc2ccccc23)c2cccc3c2[C@@]2(CC3)CCc3cccc(N(c4ccccc4)c4cccc5ccccc45)c32)cc1]

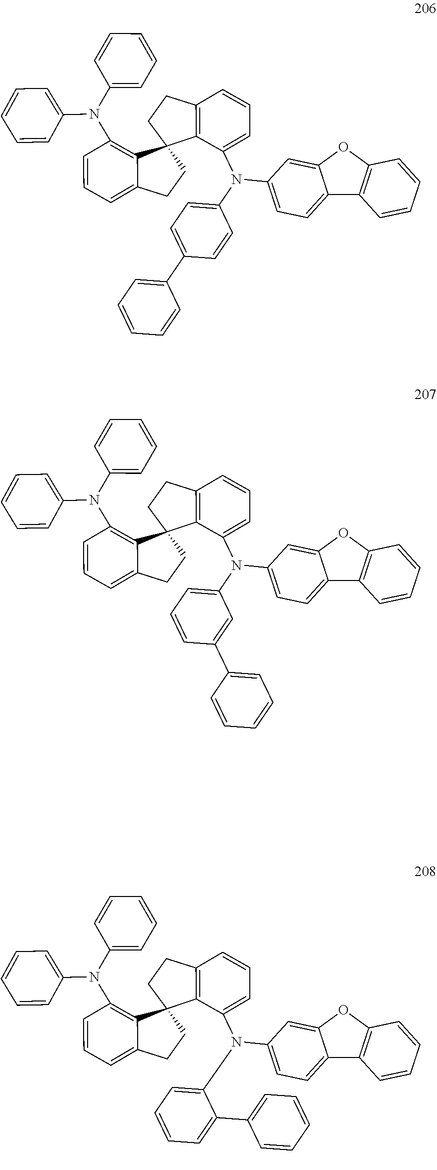

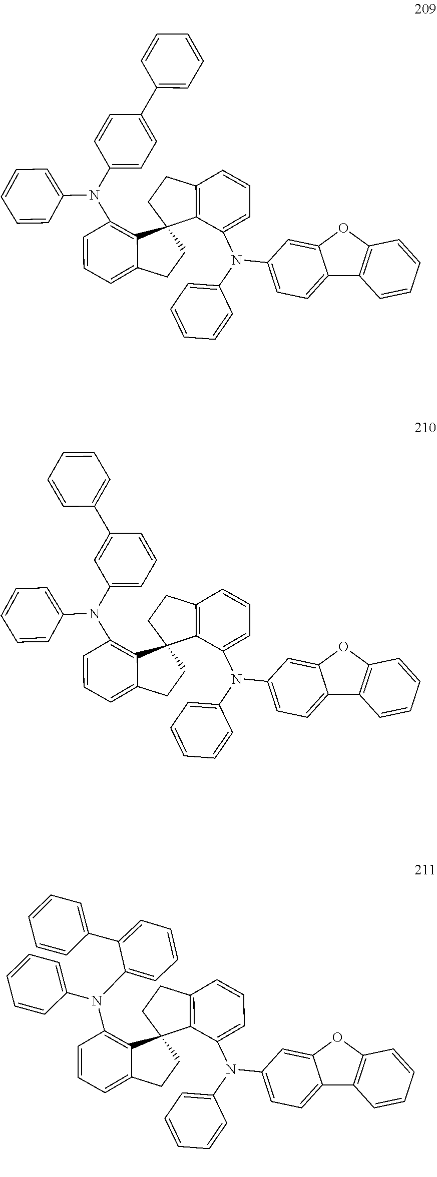

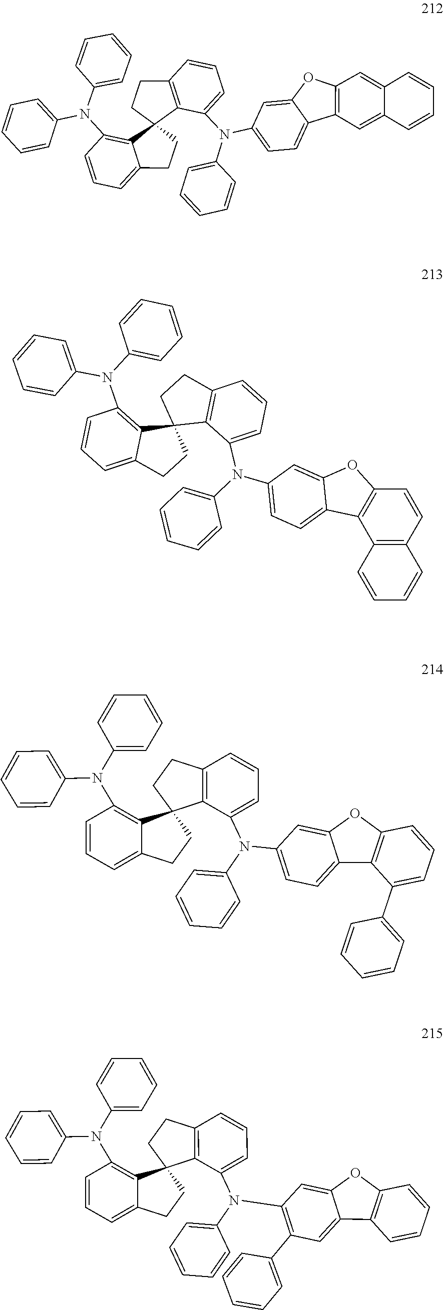 [SMILES: c1ccc(N(c2ccccc2)c2cccc3c2[C@@]2(CC3)CCc3cccc(N(c4ccccc4)c4ccc5c(c4)oc4cc6ccccc6cc45)c32)cc1 ; c1ccc(N(c2ccccc2)c2cccc3c2[C@@]2(CC3)CCc3cccc(N(c4ccccc4)c4ccc5c(c4)oc4ccc6ccccc6c45)c32)cc1 ; c1ccc(-c2cccc3oc4cc(N(c5ccccc5)c5cccc6c5[C@]5(CCc7cccc(N(c8ccccc8)c8ccccc8)c75)CC6)ccc4c23)cc1 ; c1ccc(-c2cc3c(cc2N(c2ccccc2)c2cccc4c2[C@]2(CCc5cccc(N(c6ccccc6)c6ccccc6)c52)CC4)oc2ccccc23)cc1]

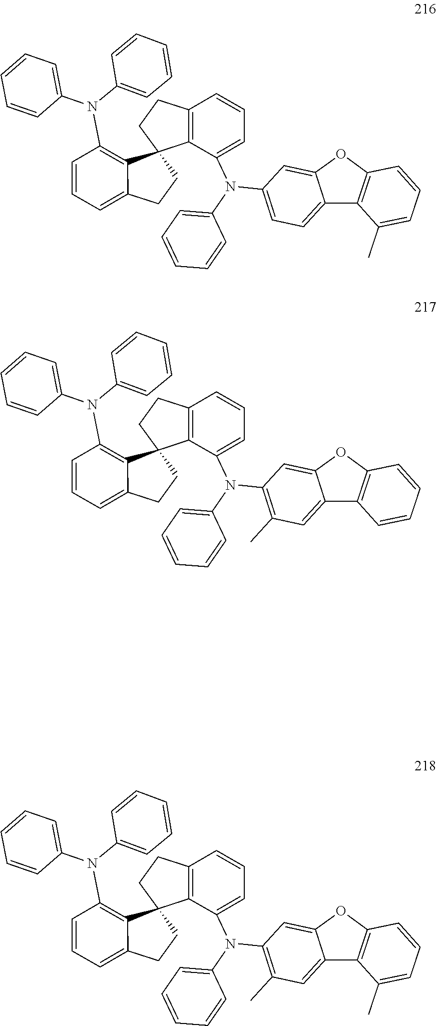 [SMILES: Cc1cccc2oc3cc(N(c4ccccc4)c4cccc5c4[C@]4(CCc6cccc(N(c7ccccc7)c7ccccc7)c64)CC5)ccc3c12 ; Cc1cc2c(cc1N(c1ccccc1)c1cccc3c1[C@]1(CCc4cccc(N(c5ccccc5)c5ccccc5)c41)CC3)oc1ccccc12 ; Cc1cc2c(cc1N(c1ccccc1)c1cccc3c1[C@]1(CCc4cccc(N(c5ccccc5)c5ccccc5)c41)CC3)oc1cccc(C)c12]

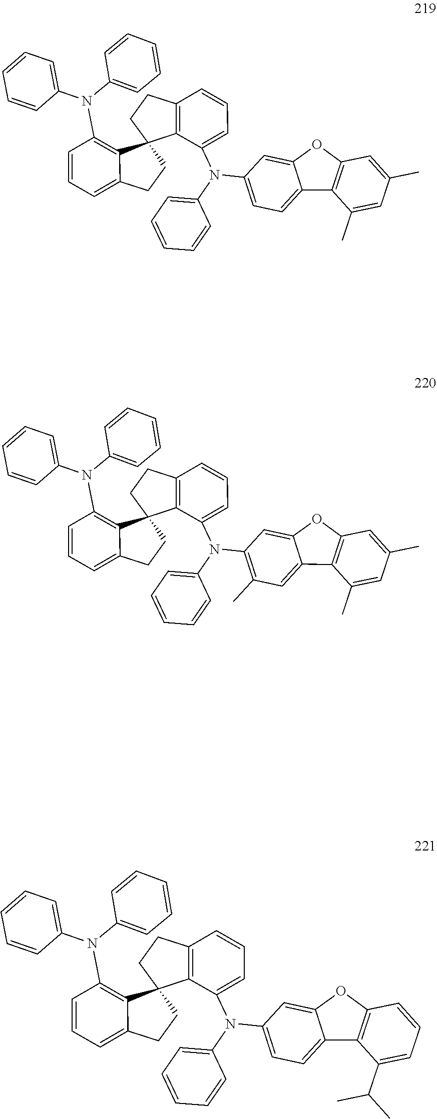 [SMILES: Cc1cc(C)c2c(c1)oc1cc(N(c3ccccc3)c3cccc4c3[C@]3(CCc5cccc(N(c6ccccc6)c6ccccc6)c53)CC4)ccc12 ; Cc1cc(C)c2c(c1)oc1cc(N(c3ccccc3)c3cccc4c3[C@]3(CCc5cccc(N(c6ccccc6)c6ccccc6)c53)CC4)c(C)cc12 ; CC(C)c1cccc2oc3cc(N(c4ccccc4)c4cccc5c4[C@]4(CCc6cccc(N(c7ccccc7)c7ccccc7)c64)CC5)ccc3c12]

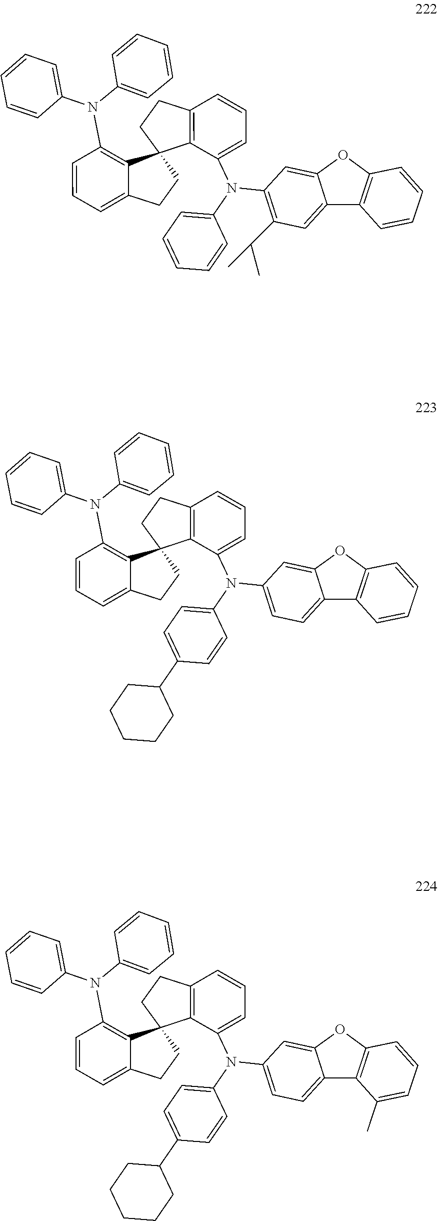 [SMILES: CC(C)c1cc2c(cc1N(c1ccccc1)c1cccc3c1[C@]1(CCc4cccc(N(c5ccccc5)c5ccccc5)c41)CC3)oc1ccccc12 ; c1ccc(N(c2ccccc2)c2cccc3c2[C@@]2(CC3)CCc3cccc(N(c4ccc(C5CCCCC5)cc4)c4ccc5c(c4)oc4ccccc45)c32)cc1 ; Cc1cccc2oc3cc(N(c4ccc(C5CCCCC5)cc4)c4cccc5c4[C@]4(CCc6cccc(N(c7ccccc7)c7ccccc7)c64)CC5)ccc3c12]

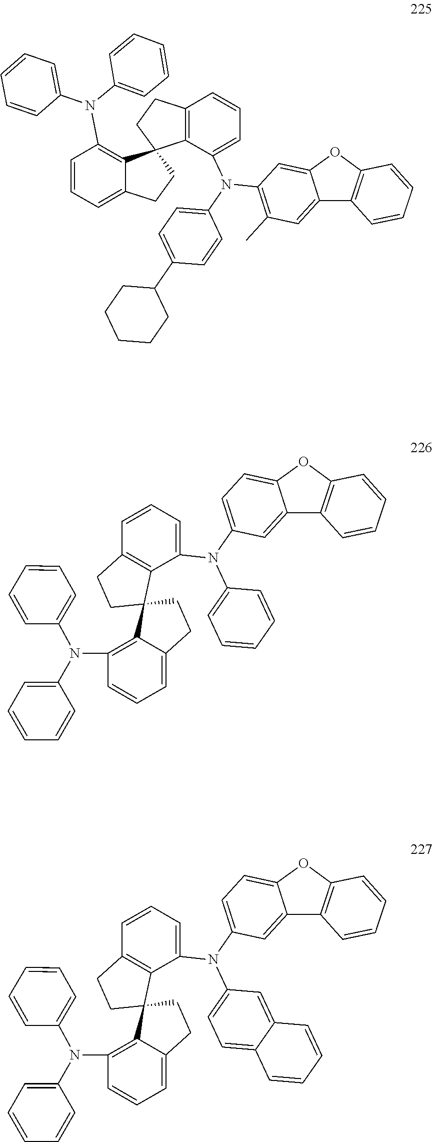 [SMILES: Cc1cc2c(cc1N(c1ccc(C3CCCCC3)cc1)c1cccc3c1[C@]1(CCc4cccc(N(c5ccccc5)c5ccccc5)c41)CC3)oc1ccccc12 ; c1ccc(N(c2ccccc2)c2cccc3c2[C@@]2(CC3)CCc3cccc(N(c4ccccc4)c4ccc5oc6ccccc6c5c4)c32)cc1 ; c1ccc(N(c2ccccc2)c2cccc3c2[C@@]2(CC3)CCc3cccc(N(c4ccc5ccccc5c4)c4ccc5oc6ccccc6c5c4)c32)cc1]

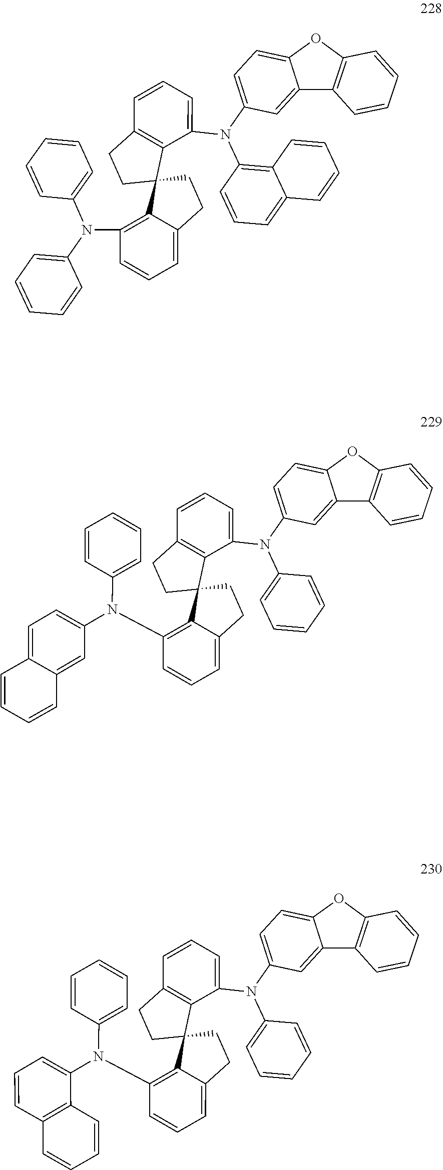 [SMILES: c1ccc(N(c2ccccc2)c2cccc3c2[C@@]2(CC3)CCc3cccc(N(c4ccc5oc6ccccc6c5c4)c4cccc5ccccc45)c32)cc1 ; c1ccc(N(c2ccc3ccccc3c2)c2cccc3c2[C@@]2(CC3)CCc3cccc(N(c4ccccc4)c4ccc5oc6ccccc6c5c4)c32)cc1 ; c1ccc(N(c2ccc3oc4ccccc4c3c2)c2cccc3c2[C@@]2(CC3)CCc3cccc(N(c4ccccc4)c4cccc5ccccc45)c32)cc1]

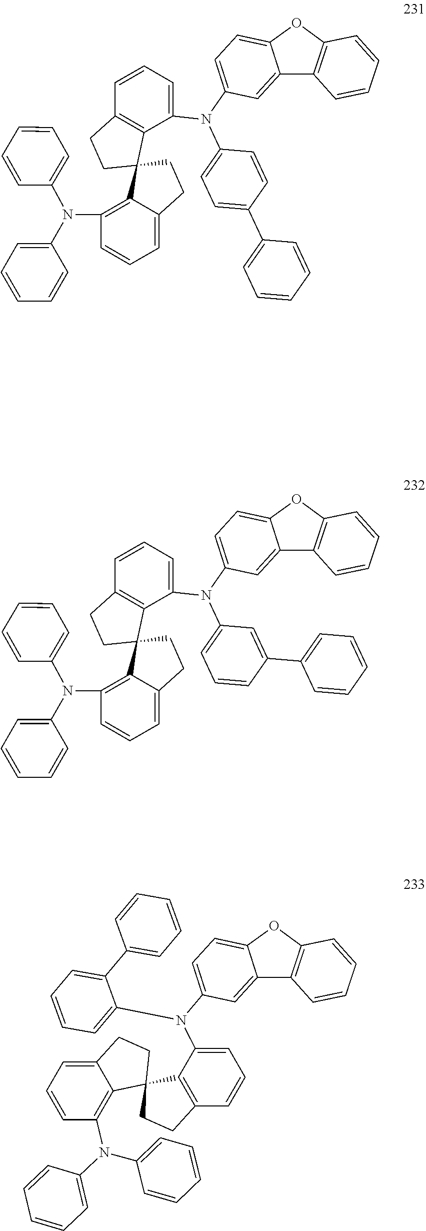 [SMILES: c1ccc(-c2ccc(N(c3ccc4oc5ccccc5c4c3)c3cccc4c3[C@]3(CCc5cccc(N(c6ccccc6)c6ccccc6)c53)CC4)cc2)cc1 ; c1ccc(-c2cccc(N(c3ccc4oc5ccccc5c4c3)c3cccc4c3[C@]3(CCc5cccc(N(c6ccccc6)c6ccccc6)c53)CC4)c2)cc1 ; c1ccc(-c2ccccc2N(c2ccc3oc4ccccc4c3c2)c2cccc3c2[C@]2(CCc4cccc(N(c5ccccc5)c5ccccc5)c42)CC3)cc1]

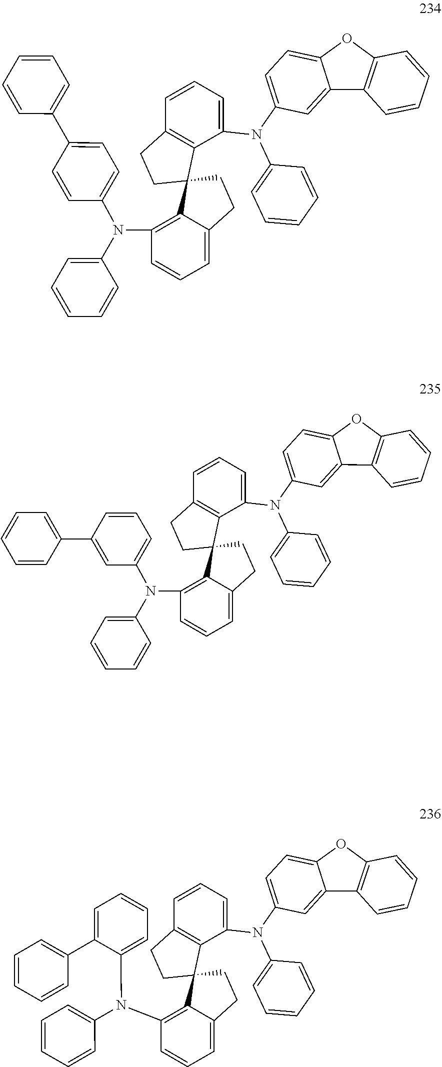 [SMILES: c1ccc(-c2ccc(N(c3ccccc3)c3cccc4c3[C@@]3(CC4)CCc4cccc(N(c5ccccc5)c5ccc6oc7ccccc7c6c5)c43)cc2)cc1 ; c1ccc(-c2cccc(N(c3ccccc3)c3cccc4c3[C@@]3(CC4)CCc4cccc(N(c5ccccc5)c5ccc6oc7ccccc7c6c5)c43)c2)cc1 ; c1ccc(-c2ccccc2N(c2ccccc2)c2cccc3c2[C@]2(CCc4cccc(N(c5ccccc5)c5ccc6oc7ccccc7c6c5)c42)CC3)cc1]

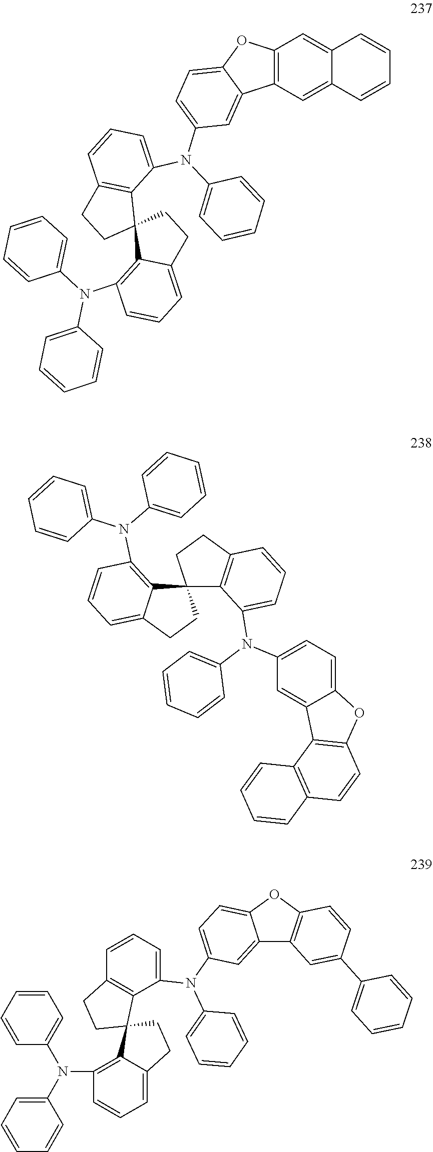 [SMILES: c1ccc(N(c2ccccc2)c2cccc3c2[C@@]2(CC3)CCc3cccc(N(c4ccccc4)c4ccc5oc6cc7ccccc7cc6c5c4)c32)cc1 ; c1ccc(N(c2ccccc2)c2cccc3c2[C@@]2(CC3)CCc3cccc(N(c4ccccc4)c4ccc5oc6ccc7ccccc7c6c5c4)c32)cc1 ; c1ccc(-c2ccc3oc4ccc(N(c5ccccc5)c5cccc6c5[C@]5(CCc7cccc(N(c8ccccc8)c8ccccc8)c75)CC6)cc4c3c2)cc1]

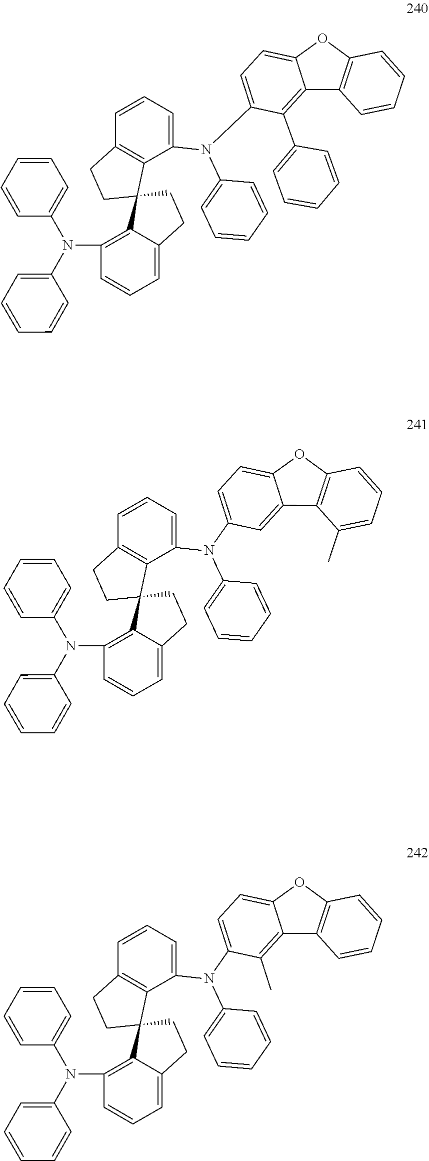 [SMILES: c1ccc(-c2c(N(c3ccccc3)c3cccc4c3[C@]3(CCc5cccc(N(c6ccccc6)c6ccccc6)c53)CC4)ccc3oc4ccccc4c23)cc1 ; Cc1cccc2oc3ccc(N(c4ccccc4)c4cccc5c4[C@]4(CCc6cccc(N(c7ccccc7)c7ccccc7)c64)CC5)cc3c12 ; Cc1c(N(c2ccccc2)c2cccc3c2[C@]2(CCc4cccc(N(c5ccccc5)c5ccccc5)c42)CC3)ccc2oc3ccccc3c12]

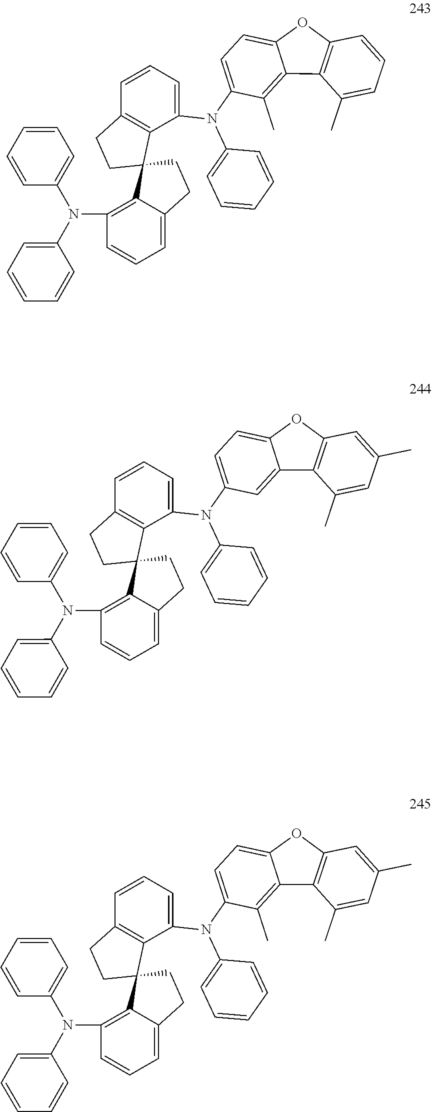 [SMILES: Cc1cccc2oc3ccc(N(c4ccccc4)c4cccc5c4[C@]4(CCc6cccc(N(c7ccccc7)c7ccccc7)c64)CC5)c(C)c3c12 ; Cc1cc(C)c2c(c1)oc1ccc(N(c3ccccc3)c3cccc4c3[C@]3(CCc5cccc(N(c6ccccc6)c6ccccc6)c53)CC4)cc12 ; Cc1cc(C)c2c(c1)oc1ccc(N(c3ccccc3)c3cccc4c3[C@]3(CCc5cccc(N(c6ccccc6)c6ccccc6)c53)CC4)c(C)c12]

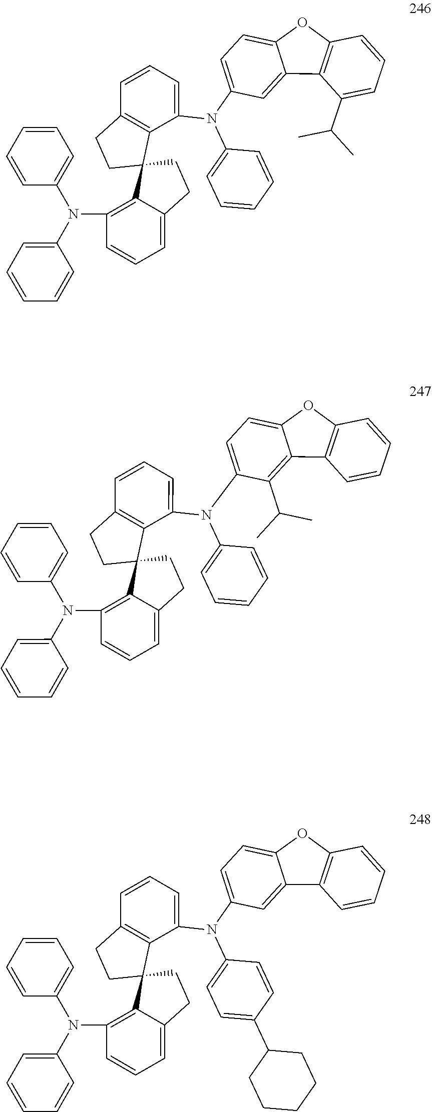 [SMILES: CC(C)c1cccc2oc3ccc(N(c4ccccc4)c4cccc5c4[C@]4(CCc6cccc(N(c7ccccc7)c7ccccc7)c64)CC5)cc3c12 ; CC(C)c1c(N(c2ccccc2)c2cccc3c2[C@]2(CCc4cccc(N(c5ccccc5)c5ccccc5)c42)CC3)ccc2oc3ccccc3c12 ; c1ccc(N(c2ccccc2)c2cccc3c2[C@@]2(CC3)CCc3cccc(N(c4ccc(C5CCCCC5)cc4)c4ccc5oc6ccccc6c5c4)c32)cc1]

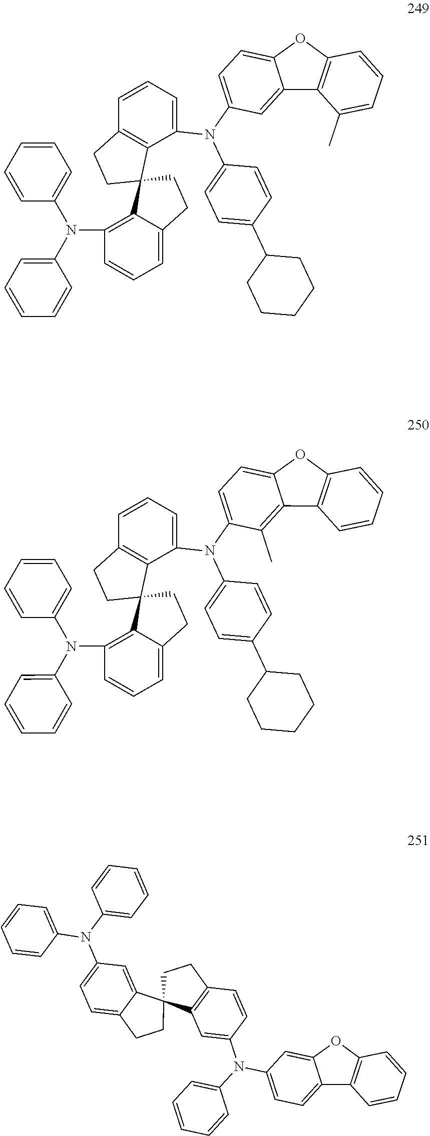 [SMILES: Cc1cccc2oc3ccc(N(c4ccc(C5CCCCC5)cc4)c4cccc5c4[C@]4(CCc6cccc(N(c7ccccc7)c7ccccc7)c64)CC5)cc3c12 ; Cc1c(N(c2ccc(C3CCCCC3)cc2)c2cccc3c2[C@]2(CCc4cccc(N(c5ccccc5)c5ccccc5)c42)CC3)ccc2oc3ccccc3c12 ; c1ccc(N(c2ccccc2)c2ccc3c(c2)[C@@]2(CC3)CCc3ccc(N(c4ccccc4)c4ccc5c(c4)oc4ccccc45)cc32)cc1]

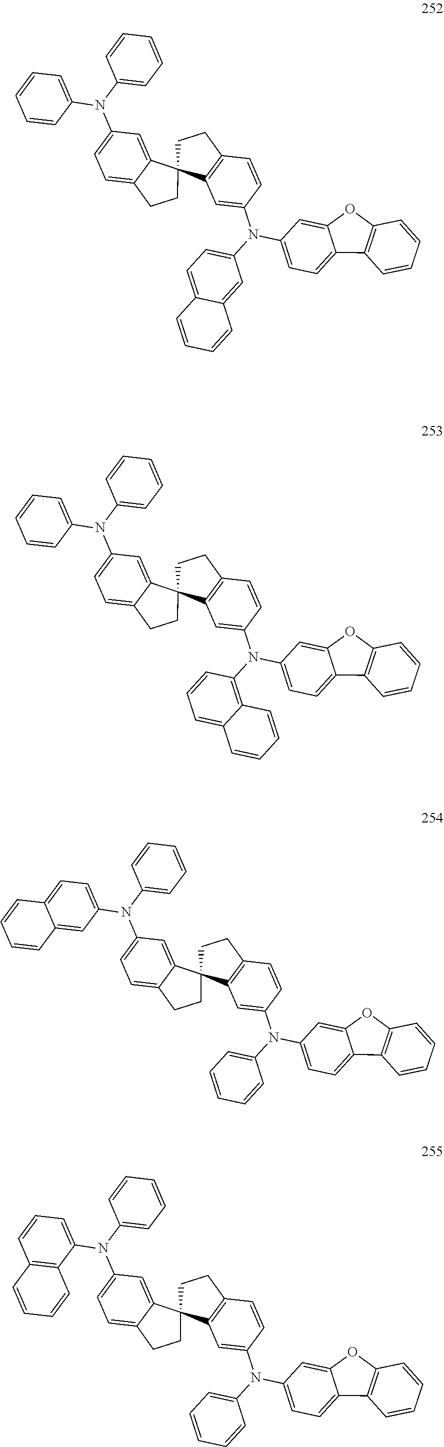 [SMILES: c1ccc(N(c2ccccc2)c2ccc3c(c2)[C@@]2(CC3)CCc3ccc(N(c4ccc5ccccc5c4)c4ccc5c(c4)oc4ccccc45)cc32)cc1 ; c1ccc(N(c2ccccc2)c2ccc3c(c2)[C@@]2(CC3)CCc3ccc(N(c4ccc5c(c4)oc4ccccc45)c4cccc5ccccc45)cc32)cc1 ; c1ccc(N(c2ccc3c(c2)[C@@]2(CC3)CCc3ccc(N(c4ccccc4)c4ccc5c(c4)oc4ccccc45)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)oc2ccccc23)cc1]

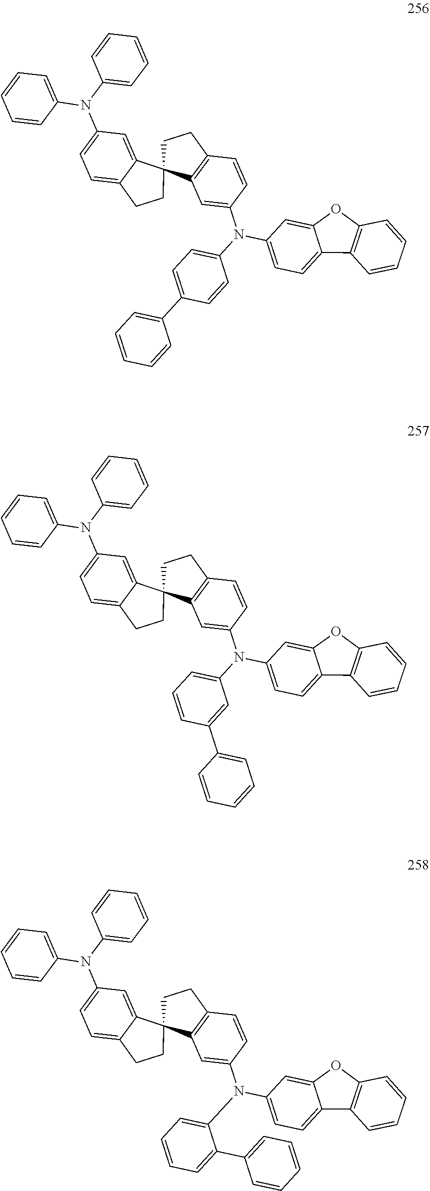 [SMILES: c1ccc(-c2ccc(N(c3ccc4c(c3)[C@]3(CCc5ccc(N(c6ccccc6)c6ccccc6)cc53)CC4)c3ccc4c(c3)oc3ccccc34)cc2)cc1 ; c1ccc(-c2cccc(N(c3ccc4c(c3)[C@]3(CCc5ccc(N(c6ccccc6)c6ccccc6)cc53)CC4)c3ccc4c(c3)oc3ccccc34)c2)cc1 ; c1ccc(-c2ccccc2N(c2ccc3c(c2)[C@]2(CCc4ccc(N(c5ccccc5)c5ccccc5)cc42)CC3)c2ccc3c(c2)oc2ccccc23)cc1]

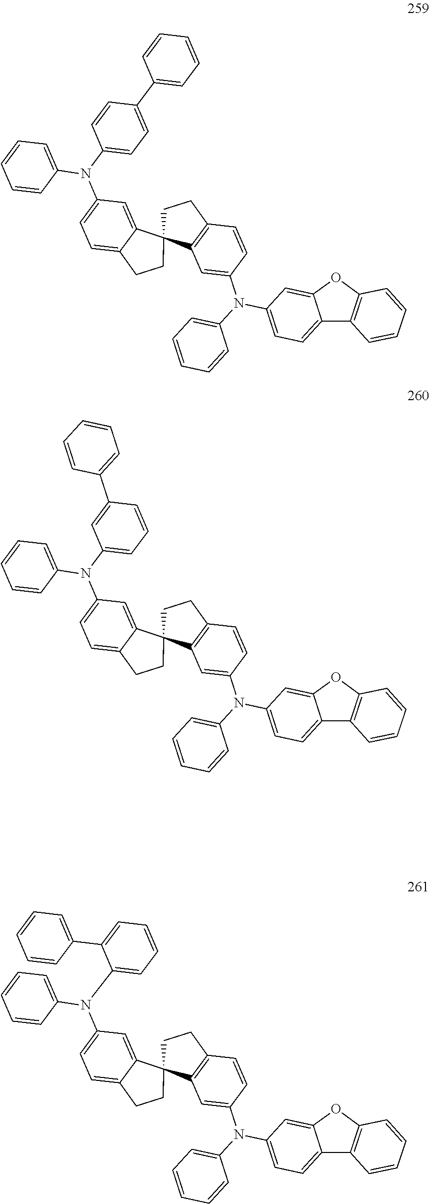 [SMILES: c1ccc(-c2ccc(N(c3ccccc3)c3ccc4c(c3)[C@@]3(CC4)CCc4ccc(N(c5ccccc5)c5ccc6c(c5)oc5ccccc56)cc43)cc2)cc1 ; c1ccc(-c2cccc(N(c3ccccc3)c3ccc4c(c3)[C@@]3(CC4)CCc4ccc(N(c5ccccc5)c5ccc6c(c5)oc5ccccc56)cc43)c2)cc1 ; c1ccc(-c2ccccc2N(c2ccccc2)c2ccc3c(c2)[C@]2(CCc4ccc(N(c5ccccc5)c5ccc6c(c5)oc5ccccc56)cc42)CC3)cc1]

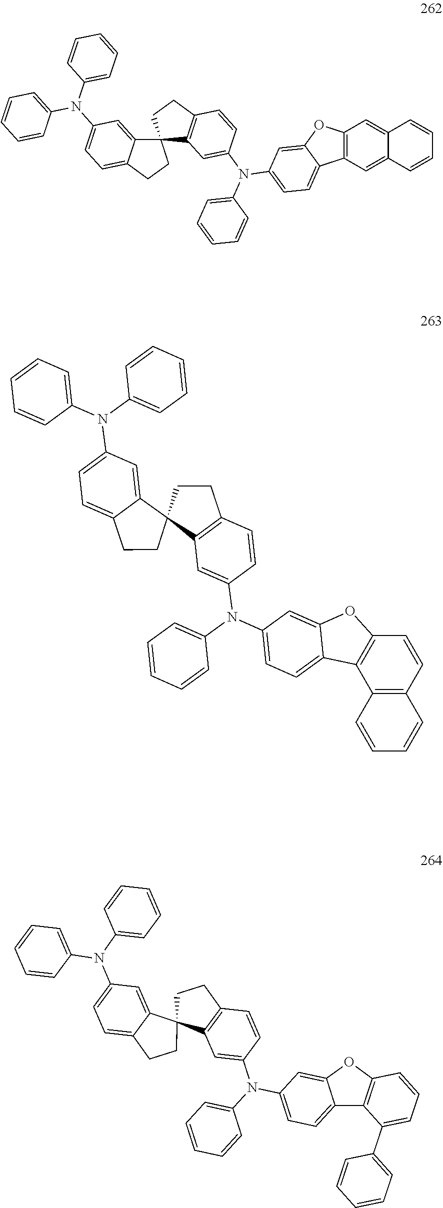 [SMILES: c1ccc(N(c2ccccc2)c2ccc3c(c2)[C@@]2(CC3)CCc3ccc(N(c4ccccc4)c4ccc5c(c4)oc4cc6ccccc6cc45)cc32)cc1 ; c1ccc(N(c2ccccc2)c2ccc3c(c2)[C@@]2(CC3)CCc3ccc(N(c4ccccc4)c4ccc5c(c4)oc4ccc6ccccc6c45)cc32)cc1 ; c1ccc(-c2cccc3oc4cc(N(c5ccccc5)c5ccc6c(c5)[C@]5(CCc7ccc(N(c8ccccc8)c8ccccc8)cc75)CC6)ccc4c23)cc1]

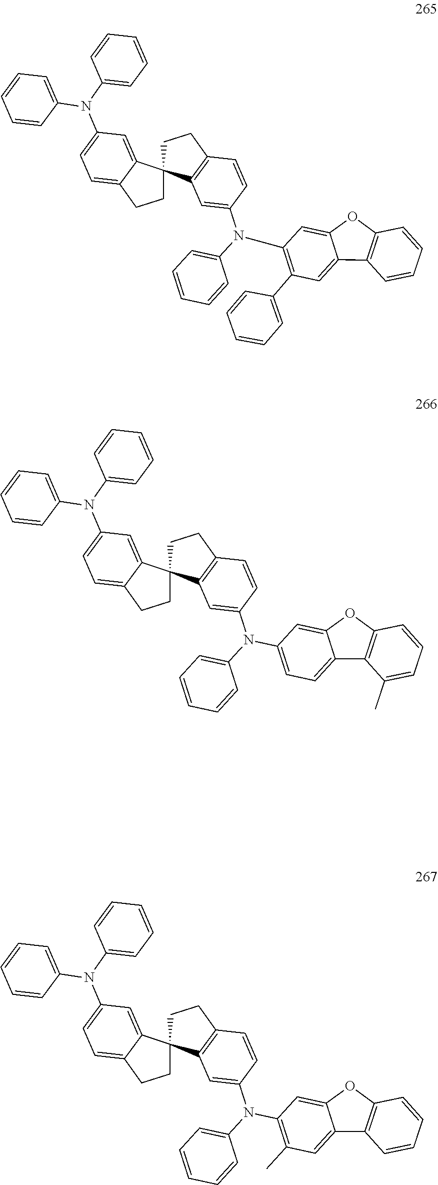 [SMILES: c1ccc(-c2cc3c(cc2N(c2ccccc2)c2ccc4c(c2)[C@]2(CCc5ccc(N(c6ccccc6)c6ccccc6)cc52)CC4)oc2ccccc23)cc1 ; Cc1cccc2oc3cc(N(c4ccccc4)c4ccc5c(c4)[C@]4(CCc6ccc(N(c7ccccc7)c7ccccc7)cc64)CC5)ccc3c12 ; Cc1cc2c(cc1N(c1ccccc1)c1ccc3c(c1)[C@]1(CCc4ccc(N(c5ccccc5)c5ccccc5)cc41)CC3)oc1ccccc12]

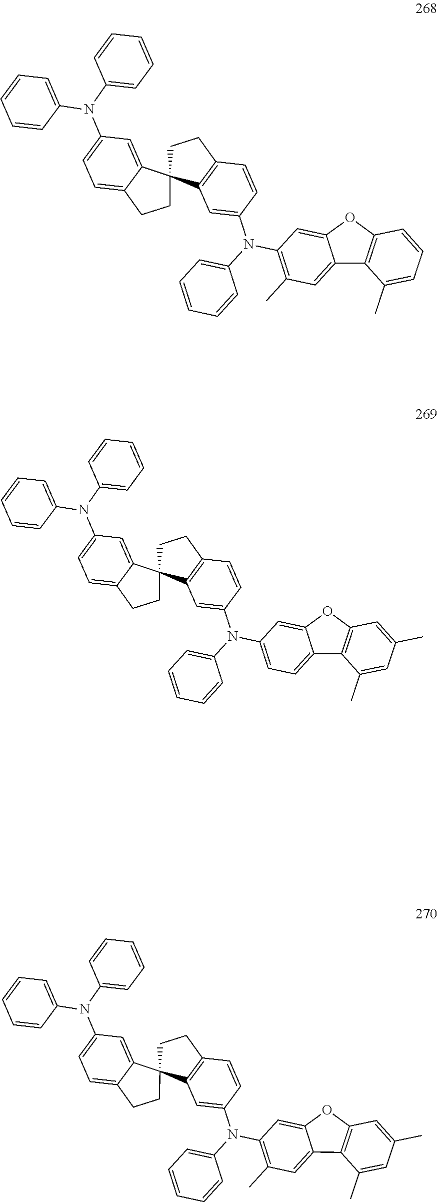 [SMILES: Cc1cc2c(cc1N(c1ccccc1)c1ccc3c(c1)[C@]1(CCc4ccc(N(c5ccccc5)c5ccccc5)cc41)CC3)oc1cccc(C)c12 ; Cc1cc(C)c2c(c1)oc1cc(N(c3ccccc3)c3ccc4c(c3)[C@]3(CCc5ccc(N(c6ccccc6)c6ccccc6)cc53)CC4)ccc12 ; Cc1cc(C)c2c(c1)oc1cc(N(c3ccccc3)c3ccc4c(c3)[C@]3(CCc5ccc(N(c6ccccc6)c6ccccc6)cc53)CC4)c(C)cc12]

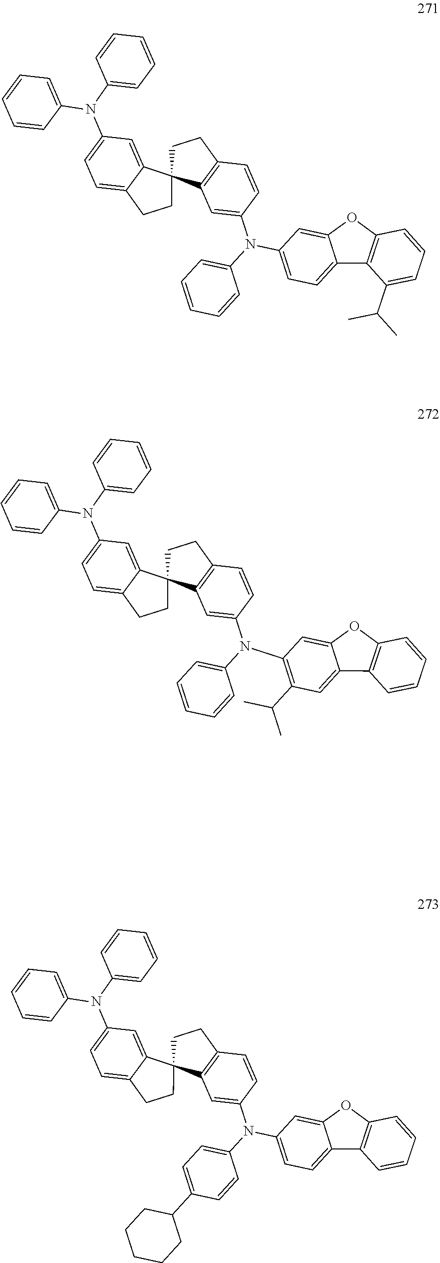 [SMILES: CC(C)c1cccc2oc3cc(N(c4ccccc4)c4ccc5c(c4)[C@]4(CCc6ccc(N(c7ccccc7)c7ccccc7)cc64)CC5)ccc3c12 ; CC(C)c1cc2c(cc1N(c1ccccc1)c1ccc3c(c1)[C@]1(CCc4ccc(N(c5ccccc5)c5ccccc5)cc41)CC3)oc1ccccc12 ; c1ccc(N(c2ccccc2)c2ccc3c(c2)[C@@]2(CC3)CCc3ccc(N(c4ccc(C5CCCCC5)cc4)c4ccc5c(c4)oc4ccccc45)cc32)cc1]

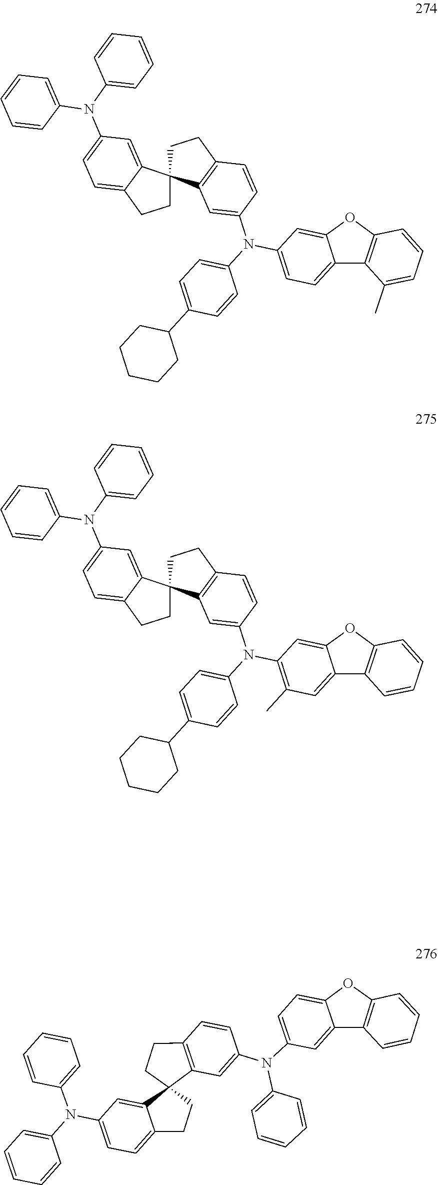 [SMILES: Cc1cccc2oc3cc(N(c4ccc(C5CCCCC5)cc4)c4ccc5c(c4)[C@]4(CCc6ccc(N(c7ccccc7)c7ccccc7)cc64)CC5)ccc3c12 ; Cc1cc2c(cc1N(c1ccc(C3CCCCC3)cc1)c1ccc3c(c1)[C@]1(CCc4ccc(N(c5ccccc5)c5ccccc5)cc41)CC3)oc1ccccc12 ; c1ccc(N(c2ccccc2)c2ccc3c(c2)[C@@]2(CC3)CCc3ccc(N(c4ccccc4)c4ccc5oc6ccccc6c5c4)cc32)cc1]

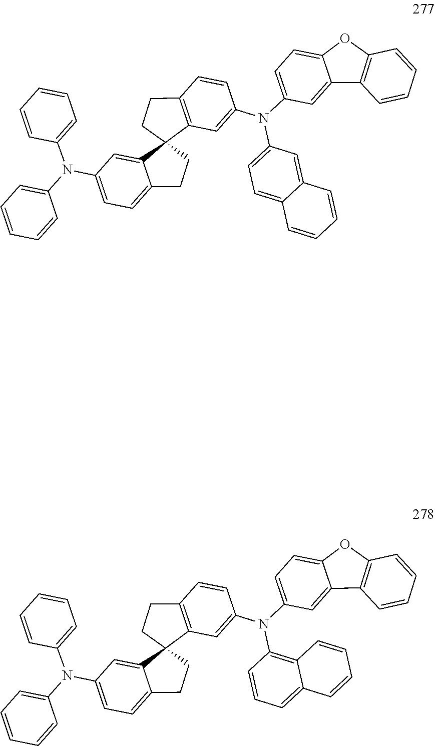 [SMILES: c1ccc(N(c2ccccc2)c2ccc3c(c2)[C@@]2(CC3)CCc3ccc(N(c4ccc5ccccc5c4)c4ccc5oc6ccccc6c5c4)cc32)cc1 ; c1ccc(N(c2ccccc2)c2ccc3c(c2)[C@@]2(CC3)CCc3ccc(N(c4ccc5oc6ccccc6c5c4)c4cccc5ccccc45)cc32)cc1]

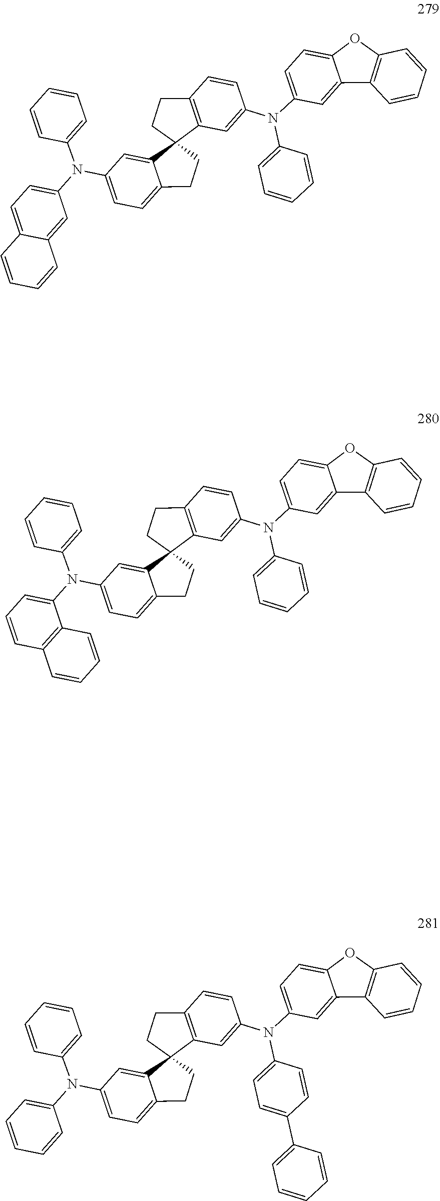 [SMILES: c1ccc(N(c2ccc3c(c2)[C@@]2(CC3)CCc3ccc(N(c4ccccc4)c4ccc5oc6ccccc6c5c4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3oc4ccccc4c3c2)cc1 ; c1ccc(-c2ccc(N(c3ccc4c(c3)[C@]3(CCc5ccc(N(c6ccccc6)c6ccccc6)cc53)CC4)c3ccc4oc5ccccc5c4c3)cc2)cc1]

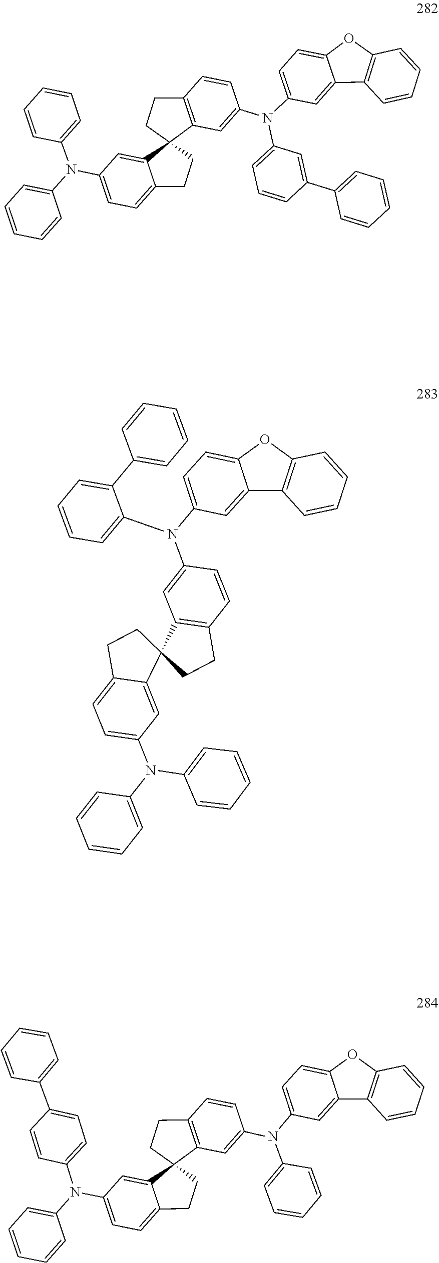 [SMILES: c1ccc(-c2cccc(N(c3ccc4c(c3)[C@]3(CCc5ccc(N(c6ccccc6)c6ccccc6)cc53)CC4)c3ccc4oc5ccccc5c4c3)c2)cc1 ; c1ccc(-c2ccccc2N(c2ccc3c(c2)[C@]2(CCc4ccc(N(c5ccccc5)c5ccccc5)cc42)CC3)c2ccc3oc4ccccc4c3c2)cc1 ; c1ccc(-c2ccc(N(c3ccccc3)c3ccc4c(c3)[C@@]3(CC4)CCc4ccc(N(c5ccccc5)c5ccc6oc7ccccc7c6c5)cc43)cc2)cc1]

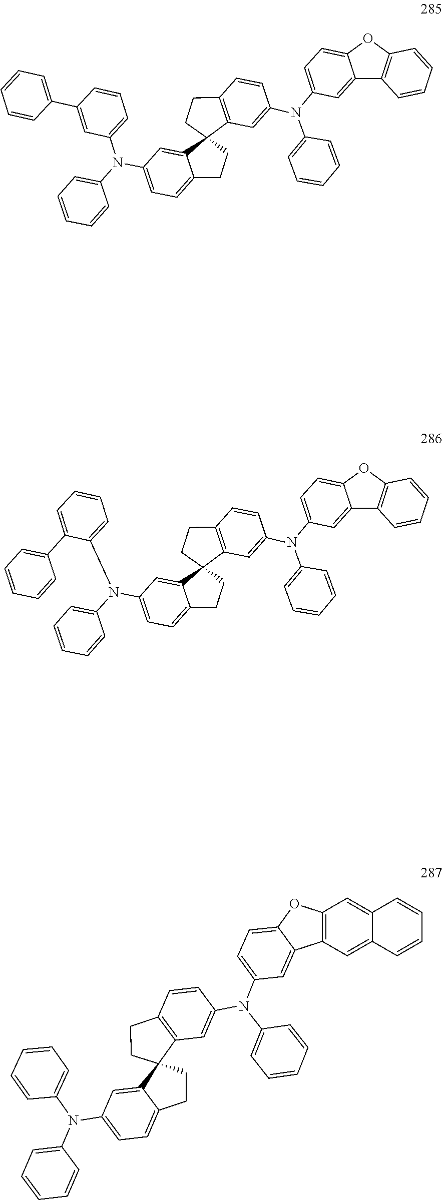 [SMILES: c1ccc(-c2cccc(N(c3ccccc3)c3ccc4c(c3)[C@@]3(CC4)CCc4ccc(N(c5ccccc5)c5ccc6oc7ccccc7c6c5)cc43)c2)cc1 ; c1ccc(-c2ccccc2N(c2ccccc2)c2ccc3c(c2)[C@]2(CCc4ccc(N(c5ccccc5)c5ccc6oc7ccccc7c6c5)cc42)CC3)cc1 ; c1ccc(N(c2ccccc2)c2ccc3c(c2)[C@@]2(CC3)CCc3ccc(N(c4ccccc4)c4ccc5oc6cc7ccccc7cc6c5c4)cc32)cc1]

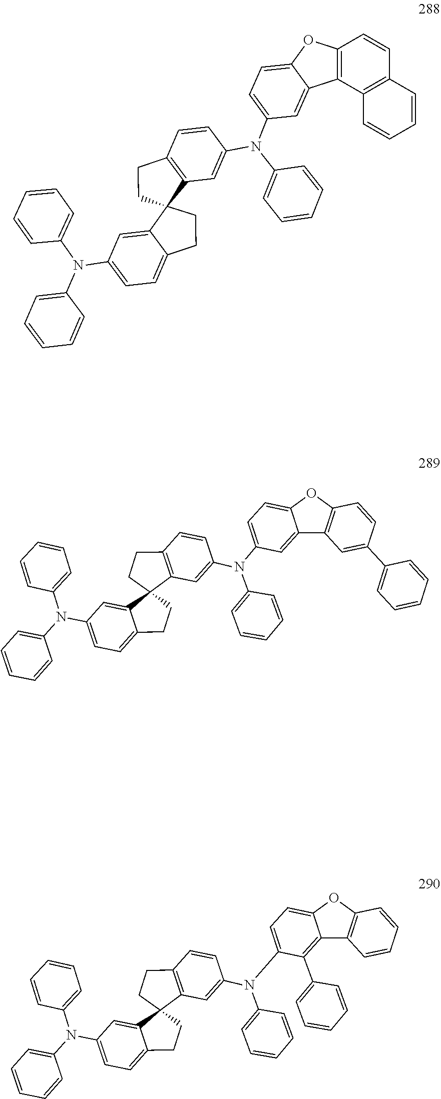 [SMILES: c1ccc(N(c2ccccc2)c2ccc3c(c2)[C@@]2(CC3)CCc3ccc(N(c4ccccc4)c4ccc5oc6ccc7ccccc7c6c5c4)cc32)cc1 ; c1ccc(-c2ccc3oc4ccc(N(c5ccccc5)c5ccc6c(c5)[C@]5(CCc7ccc(N(c8ccccc8)c8ccccc8)cc75)CC6)cc4c3c2)cc1 ; c1ccc(-c2c(N(c3ccccc3)c3ccc4c(c3)[C@]3(CCc5ccc(N(c6ccccc6)c6ccccc6)cc53)CC4)ccc3oc4ccccc4c23)cc1]

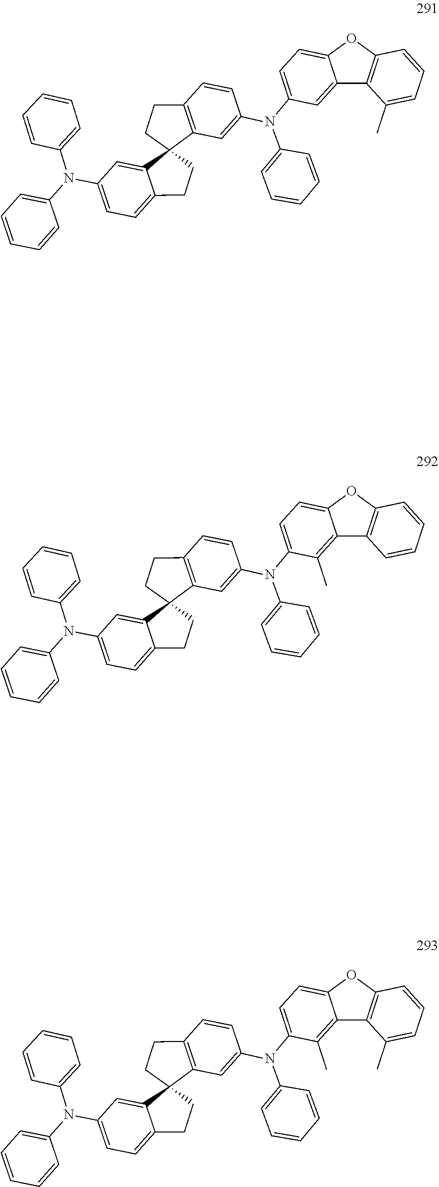 [SMILES: Cc1cccc2oc3ccc(N(c4ccccc4)c4ccc5c(c4)[C@]4(CCc6ccc(N(c7ccccc7)c7ccccc7)cc64)CC5)cc3c12 ; Cc1c(N(c2ccccc2)c2ccc3c(c2)[C@]2(CCc4ccc(N(c5ccccc5)c5ccccc5)cc42)CC3)ccc2oc3ccccc3c12 ; Cc1cccc2oc3ccc(N(c4ccccc4)c4ccc5c(c4)[C@]4(CCc6ccc(N(c7ccccc7)c7ccccc7)cc64)CC5)c(C)c3c12]

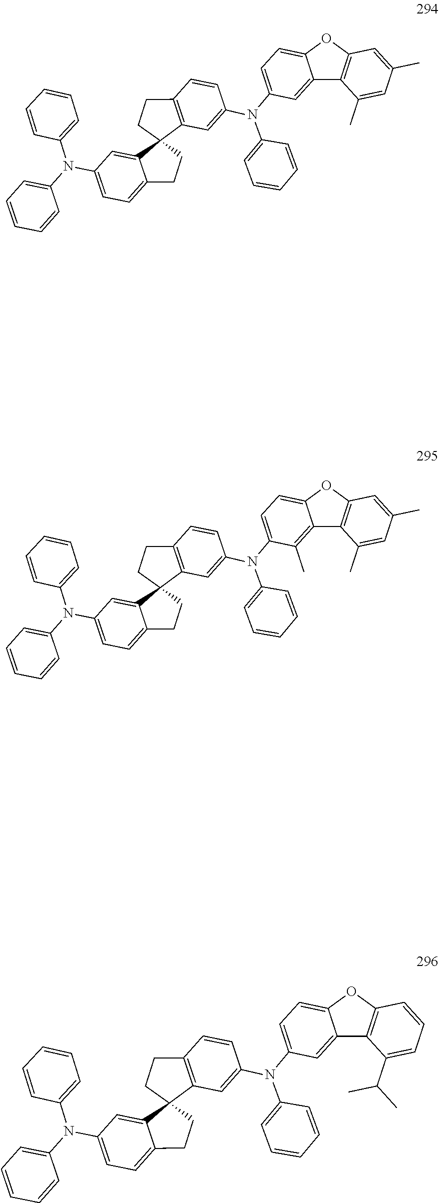 [SMILES: Cc1cc(C)c2c(c1)oc1ccc(N(c3ccccc3)c3ccc4c(c3)[C@]3(CCc5ccc(N(c6ccccc6)c6ccccc6)cc53)CC4)cc12 ; Cc1cc(C)c2c(c1)oc1ccc(N(c3ccccc3)c3ccc4c(c3)[C@]3(CCc5ccc(N(c6ccccc6)c6ccccc6)cc53)CC4)c(C)c12 ; CC(C)c1cccc2oc3ccc(N(c4ccccc4)c4ccc5c(c4)[C@]4(CCc6ccc(N(c7ccccc7)c7ccccc7)cc64)CC5)cc3c12]

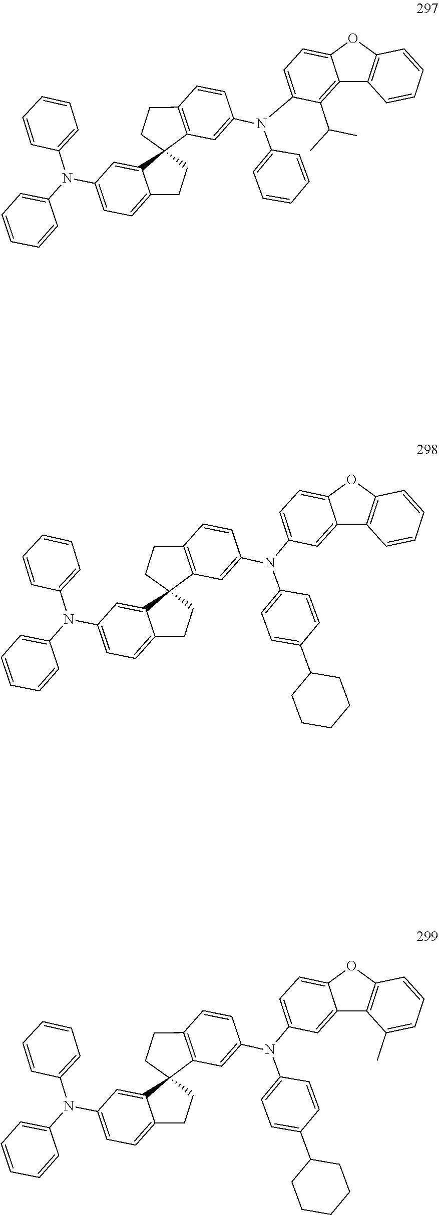 [SMILES: CC(C)c1c(N(c2ccccc2)c2ccc3c(c2)[C@]2(CCc4ccc(N(c5ccccc5)c5ccccc5)cc42)CC3)ccc2oc3ccccc3c12 ; c1ccc(N(c2ccccc2)c2ccc3c(c2)[C@@]2(CC3)CCc3ccc(N(c4ccc(C5CCCCC5)cc4)c4ccc5oc6ccccc6c5c4)cc32)cc1 ; Cc1cccc2oc3ccc(N(c4ccc(C5CCCCC5)cc4)c4ccc5c(c4)[C@]4(CCc6ccc(N(c7ccccc7)c7ccccc7)cc64)CC5)cc3c12]

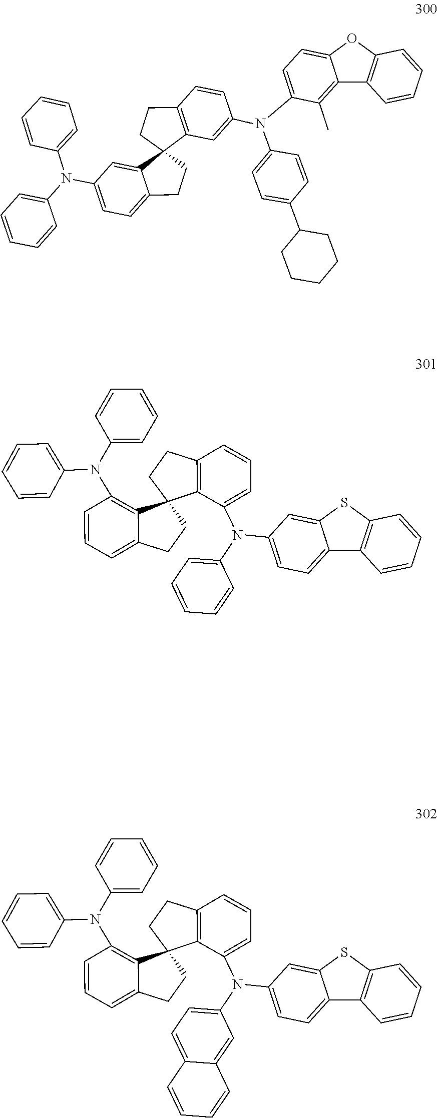 [SMILES: Cc1c(N(c2ccc(C3CCCCC3)cc2)c2ccc3c(c2)[C@]2(CCc4ccc(N(c5ccccc5)c5ccccc5)cc42)CC3)ccc2oc3ccccc3c12 ; c1ccc(N(c2ccccc2)c2cccc3c2[C@@]2(CC3)CCc3cccc(N(c4ccccc4)c4ccc5c(c4)sc4ccccc45)c32)cc1 ; c1ccc(N(c2ccccc2)c2cccc3c2[C@@]2(CC3)CCc3cccc(N(c4ccc5ccccc5c4)c4ccc5c(c4)sc4ccccc45)c32)cc1]

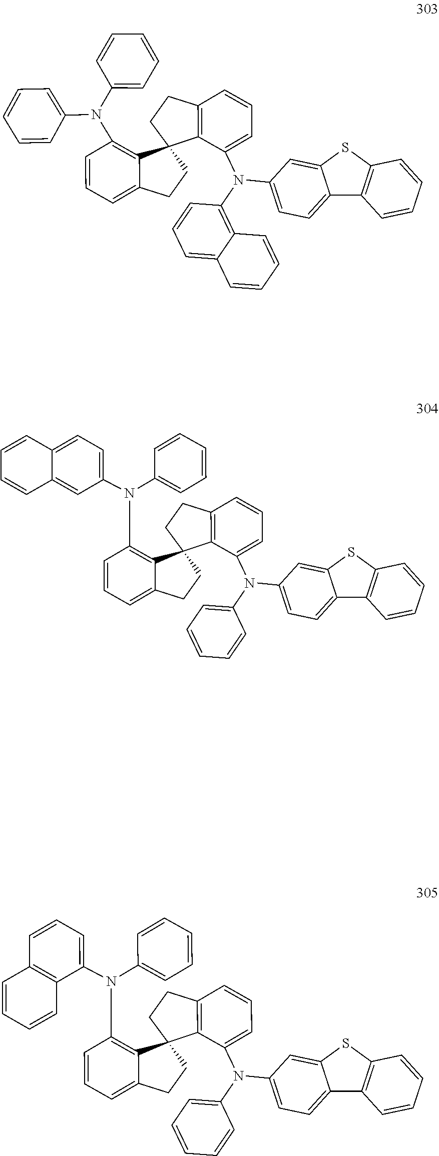 [SMILES: c1ccc(N(c2ccccc2)c2cccc3c2[C@@]2(CC3)CCc3cccc(N(c4ccc5c(c4)sc4ccccc45)c4cccc5ccccc45)c32)cc1 ; c1ccc(N(c2ccc3ccccc3c2)c2cccc3c2[C@@]2(CC3)CCc3cccc(N(c4ccccc4)c4ccc5c(c4)sc4ccccc45)c32)cc1 ; c1ccc(N(c2ccc3c(c2)sc2ccccc23)c2cccc3c2[C@@]2(CC3)CCc3cccc(N(c4ccccc4)c4cccc5ccccc45)c32)cc1]

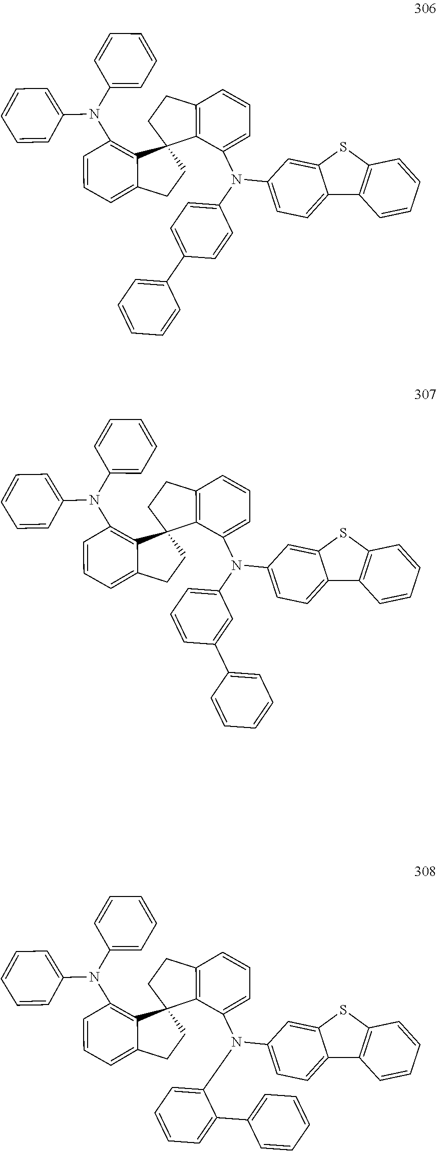

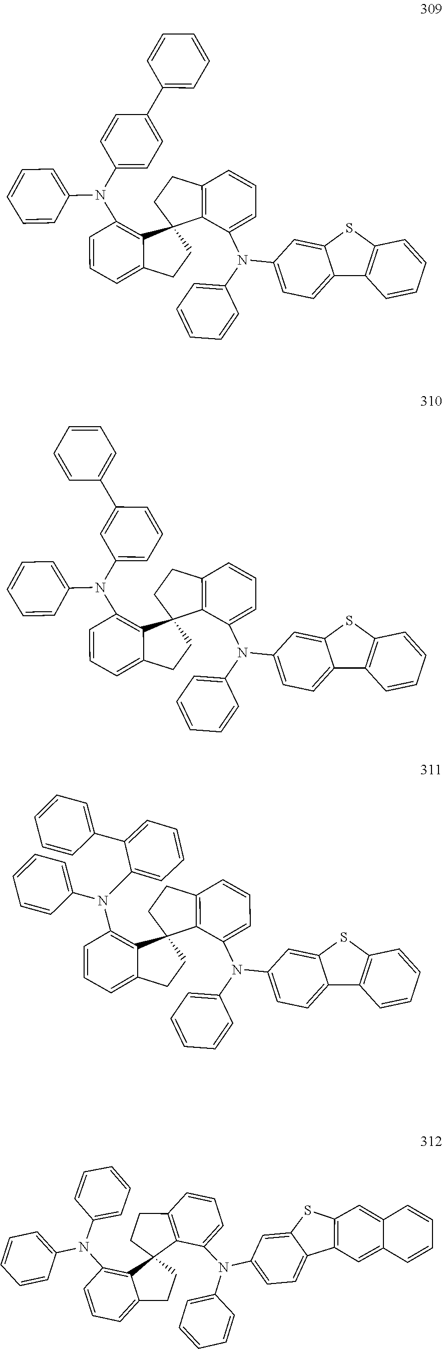

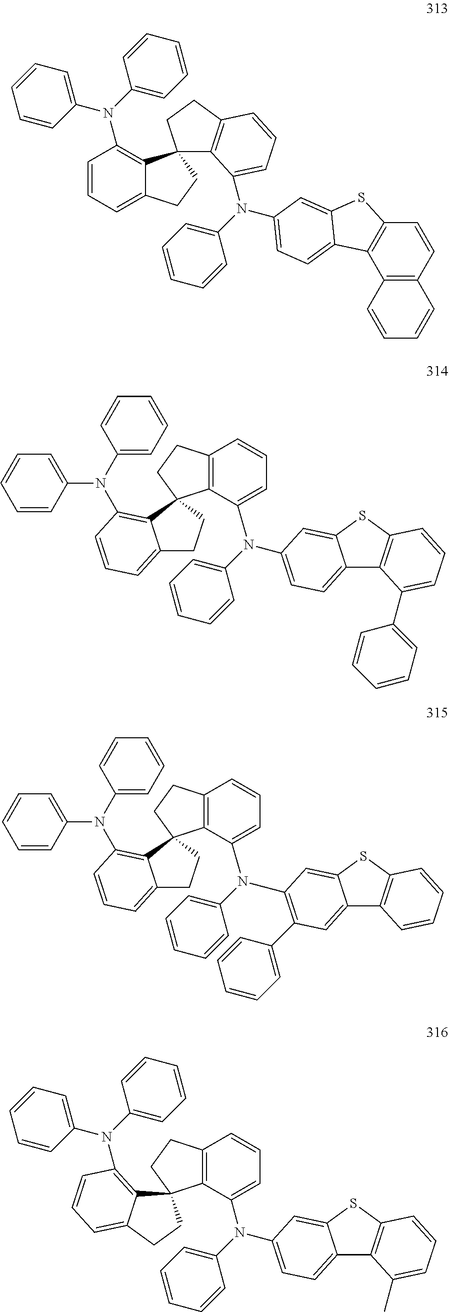 [SMILES: c1ccc(N(c2ccccc2)c2cccc3c2[C@@]2(CC3)CCc3cccc(N(c4ccccc4)c4ccc5c(c4)sc4ccc6ccccc6c45)c32)cc1 ; c1ccc(-c2cccc3sc4cc(N(c5ccccc5)c5cccc6c5[C@]5(CCc7cccc(N(c8ccccc8)c8ccccc8)c75)CC6)ccc4c23)cc1 ; c1ccc(-c2cc3c(cc2N(c2ccccc2)c2cccc4c2[C@]2(CCc5cccc(N(c6ccccc6)c6ccccc6)c52)CC4)sc2ccccc23)cc1 ; Cc1cccc2sc3cc(N(c4ccccc4)c4cccc5c4[C@]4(CCc6cccc(N(c7ccccc7)c7ccccc7)c64)CC5)ccc3c12]

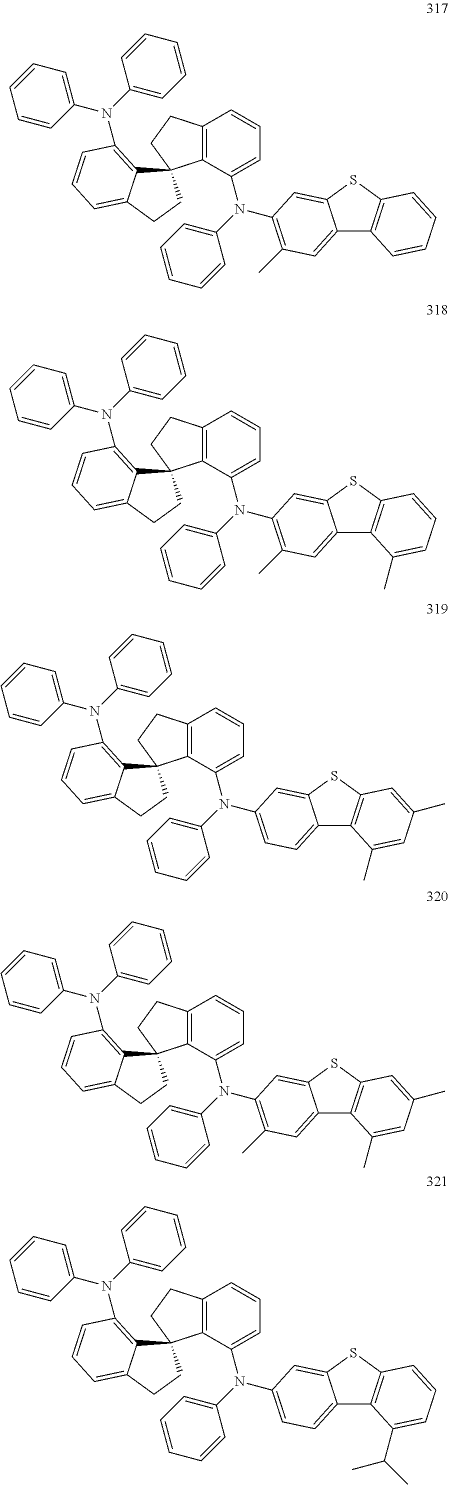 [SMILES: Cc1cc2c(cc1N(c1ccccc1)c1cccc3c1[C@]1(CCc4cccc(N(c5ccccc5)c5ccccc5)c41)CC3)sc1ccccc12 ; Cc1cc2c(cc1N(c1ccccc1)c1cccc3c1[C@]1(CCc4cccc(N(c5ccccc5)c5ccccc5)c41)CC3)sc1cccc(C)c12 ; Cc1cc(C)c2c(c1)sc1cc(N(c3ccccc3)c3cccc4c3[C@]3(CCc5cccc(N(c6ccccc6)c6ccccc6)c53)CC4)ccc12 ; Cc1cc(C)c2c(c1)sc1cc(N(c3ccccc3)c3cccc4c3[C@]3(CCc5cccc(N(c6ccccc6)c6ccccc6)c53)CC4)c(C)cc12 ; CC(C)c1cccc2sc3cc(N(c4ccccc4)c4cccc5c4[C@]4(CCc6cccc(N(c7ccccc7)c7ccccc7)c64)CC5)ccc3c12]

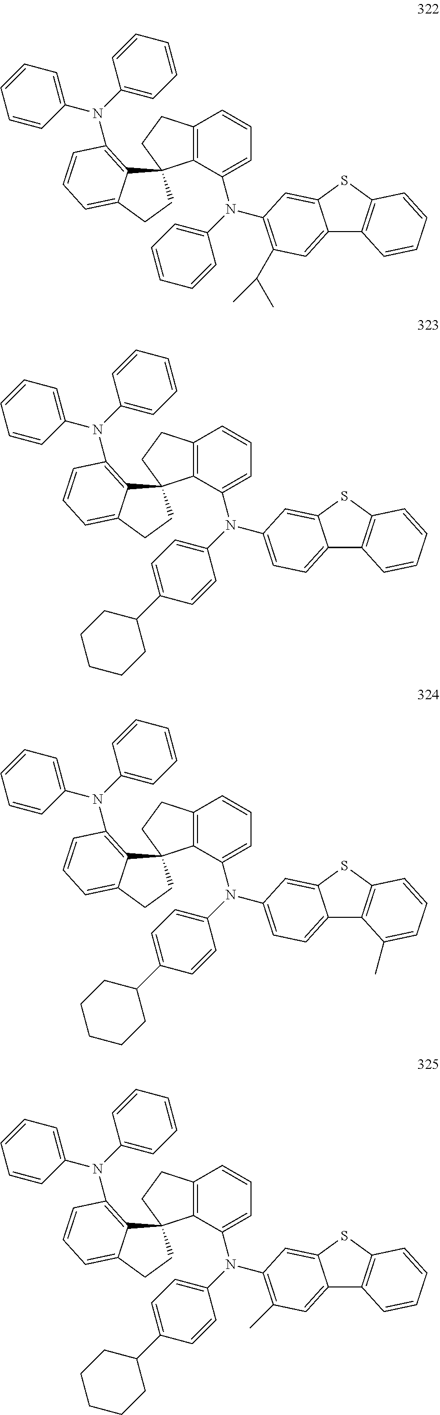 [SMILES: CC(C)c1cc2c(cc1N(c1ccccc1)c1cccc3c1[C@]1(CCc4cccc(N(c5ccccc5)c5ccccc5)c41)CC3)sc1ccccc12 ; c1ccc(N(c2ccccc2)c2cccc3c2[C@@]2(CC3)CCc3cccc(N(c4ccc(C5CCCCC5)cc4)c4ccc5c(c4)sc4ccccc45)c32)cc1 ; Cc1cccc2sc3cc(N(c4ccc(C5CCCCC5)cc4)c4cccc5c4[C@]4(CCc6cccc(N(c7ccccc7)c7ccccc7)c64)CC5)ccc3c12 ; Cc1cc2c(cc1N(c1ccc(C3CCCCC3)cc1)c1cccc3c1[C@]1(CCc4cccc(N(c5ccccc5)c5ccccc5)c41)CC3)sc1ccccc12]

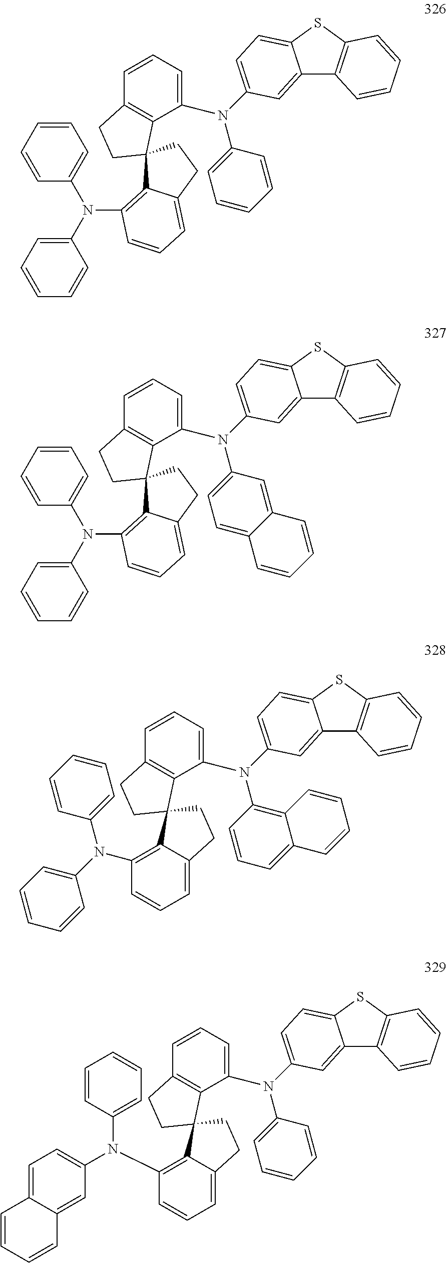 [SMILES: c1ccc(N(c2ccccc2)c2cccc3c2[C@@]2(CC3)CCc3cccc(N(c4ccccc4)c4ccc5sc6ccccc6c5c4)c32)cc1 ; c1ccc(N(c2ccccc2)c2cccc3c2[C@@]2(CC3)CCc3cccc(N(c4ccc5ccccc5c4)c4ccc5sc6ccccc6c5c4)c32)cc1 ; c1ccc(N(c2ccccc2)c2cccc3c2[C@@]2(CC3)CCc3cccc(N(c4ccc5sc6ccccc6c5c4)c4cccc5ccccc45)c32)cc1 ; c1ccc(N(c2ccc3ccccc3c2)c2cccc3c2[C@@]2(CC3)CCc3cccc(N(c4ccccc4)c4ccc5sc6ccccc6c5c4)c32)cc1]

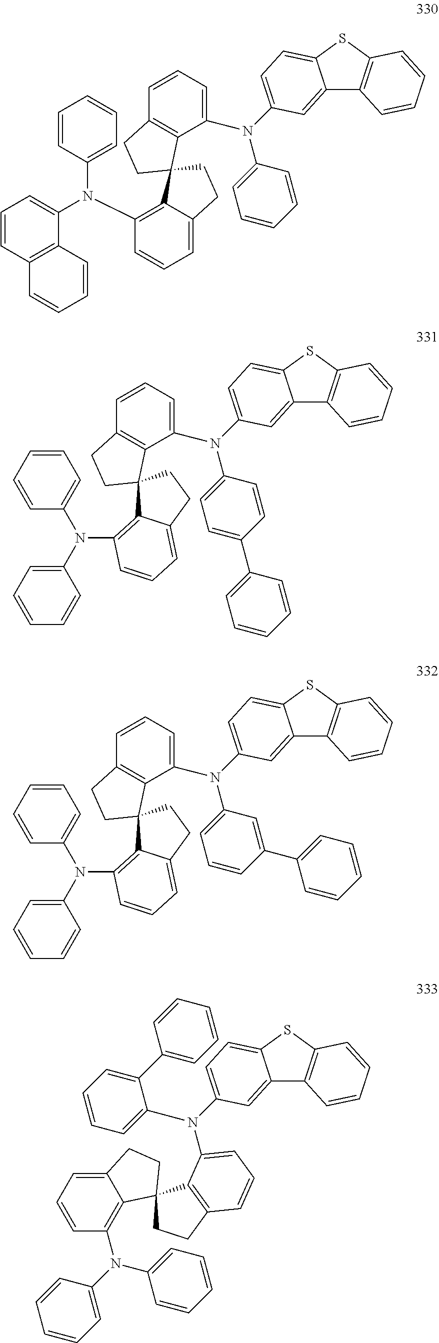 [SMILES: c1ccc(N(c2ccc3sc4ccccc4c3c2)c2cccc3c2[C@@]2(CC3)CCc3cccc(N(c4ccccc4)c4cccc5ccccc45)c32)cc1 ; c1ccc(-c2ccc(N(c3ccc4sc5ccccc5c4c3)c3cccc4c3[C@]3(CCc5cccc(N(c6ccccc6)c6ccccc6)c53)CC4)cc2)cc1 ; c1ccc(-c2cccc(N(c3ccc4sc5ccccc5c4c3)c3cccc4c3[C@]3(CCc5cccc(N(c6ccccc6)c6ccccc6)c53)CC4)c2)cc1 ; c1ccc(-c2ccccc2N(c2ccc3sc4ccccc4c3c2)c2cccc3c2[C@]2(CCc4cccc(N(c5ccccc5)c5ccccc5)c42)CC3)cc1]

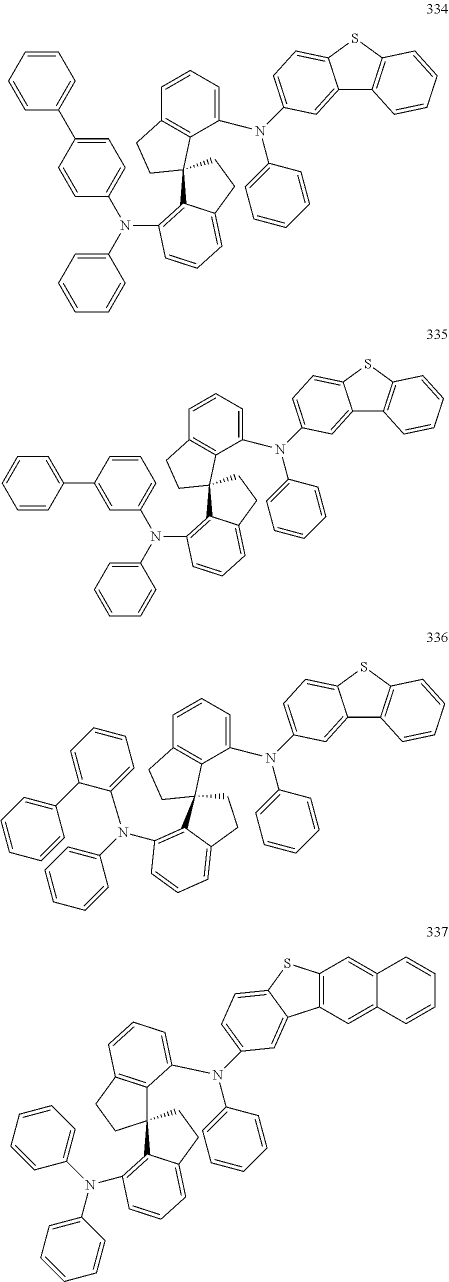 [SMILES: c1ccc(-c2ccc(N(c3ccccc3)c3cccc4c3[C@@]3(CC4)CCc4cccc(N(c5ccccc5)c5ccc6sc7ccccc7c6c5)c43)cc2)cc1 ; c1ccc(-c2cccc(N(c3ccccc3)c3cccc4c3[C@@]3(CC4)CCc4cccc(N(c5ccccc5)c5ccc6sc7ccccc7c6c5)c43)c2)cc1 ; c1ccc(-c2ccccc2N(c2ccccc2)c2cccc3c2[C@]2(CCc4cccc(N(c5ccccc5)c5ccc6sc7ccccc7c6c5)c42)CC3)cc1 ; c1ccc(N(c2ccccc2)c2cccc3c2[C@@]2(CC3)CCc3cccc(N(c4ccccc4)c4ccc5sc6cc7ccccc7cc6c5c4)c32)cc1]

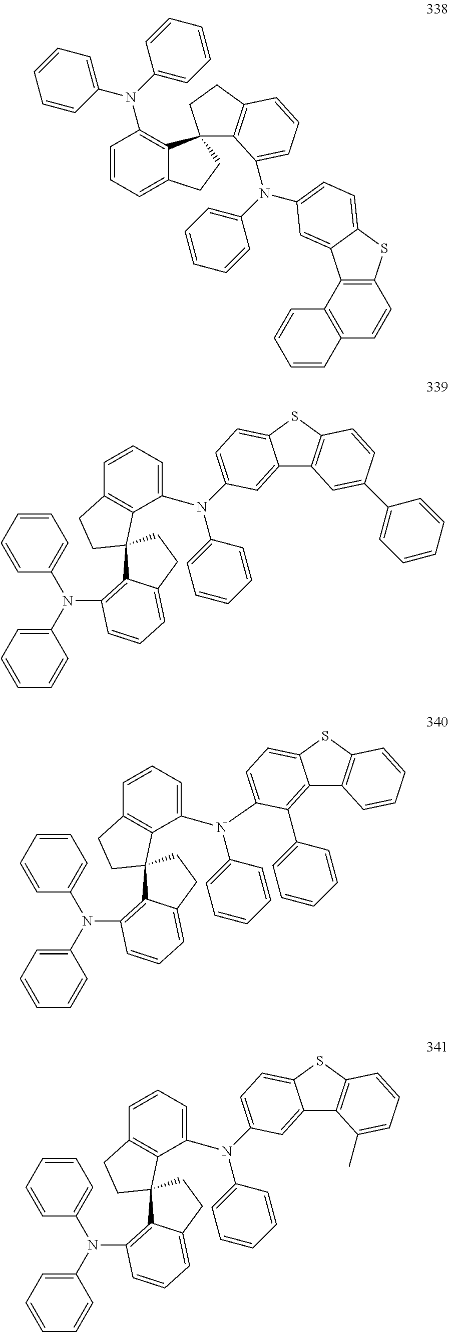 [SMILES: c1ccc(N(c2ccccc2)c2cccc3c2[C@@]2(CC3)CCc3cccc(N(c4ccccc4)c4ccc5sc6ccc7ccccc7c6c5c4)c32)cc1 ; c1ccc(-c2ccc3sc4ccc(N(c5ccccc5)c5cccc6c5[C@]5(CCc7cccc(N(c8ccccc8)c8ccccc8)c75)CC6)cc4c3c2)cc1 ; c1ccc(-c2c(N(c3ccccc3)c3cccc4c3[C@]3(CCc5cccc(N(c6ccccc6)c6ccccc6)c53)CC4)ccc3sc4ccccc4c23)cc1 ; Cc1cccc2sc3ccc(N(c4ccccc4)c4cccc5c4[C@]4(CCc6cccc(N(c7ccccc7)c7ccccc7)c64)CC5)cc3c12]

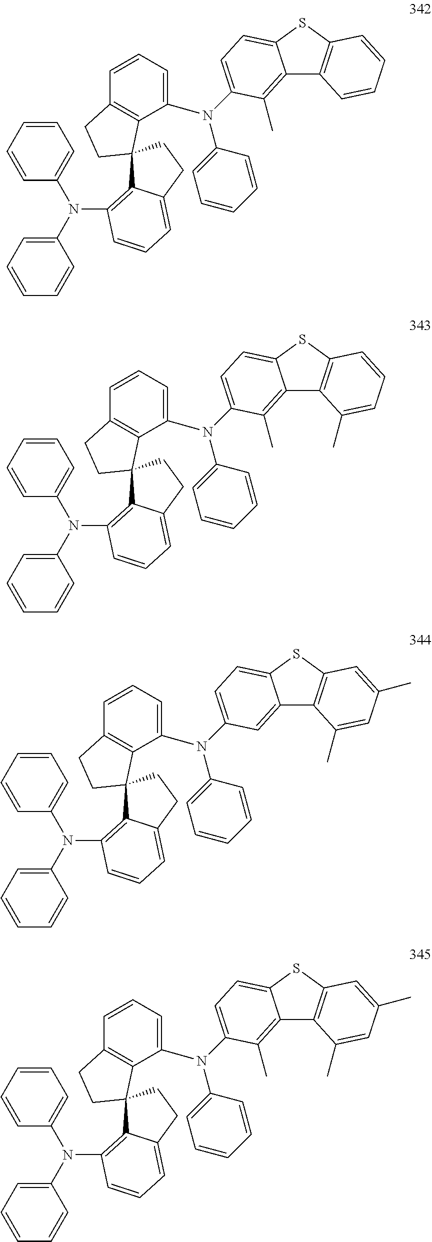 [SMILES: Cc1c(N(c2ccccc2)c2cccc3c2[C@]2(CCc4cccc(N(c5ccccc5)c5ccccc5)c42)CC3)ccc2sc3ccccc3c12 ; Cc1cccc2sc3ccc(N(c4ccccc4)c4cccc5c4[C@]4(CCc6cccc(N(c7ccccc7)c7ccccc7)c64)CC5)c(C)c3c12 ; Cc1cc(C)c2c(c1)sc1ccc(N(c3ccccc3)c3cccc4c3[C@]3(CCc5cccc(N(c6ccccc6)c6ccccc6)c53)CC4)cc12 ; Cc1cc(C)c2c(c1)sc1ccc(N(c3ccccc3)c3cccc4c3[C@]3(CCc5cccc(N(c6ccccc6)c6ccccc6)c53)CC4)c(C)c12]

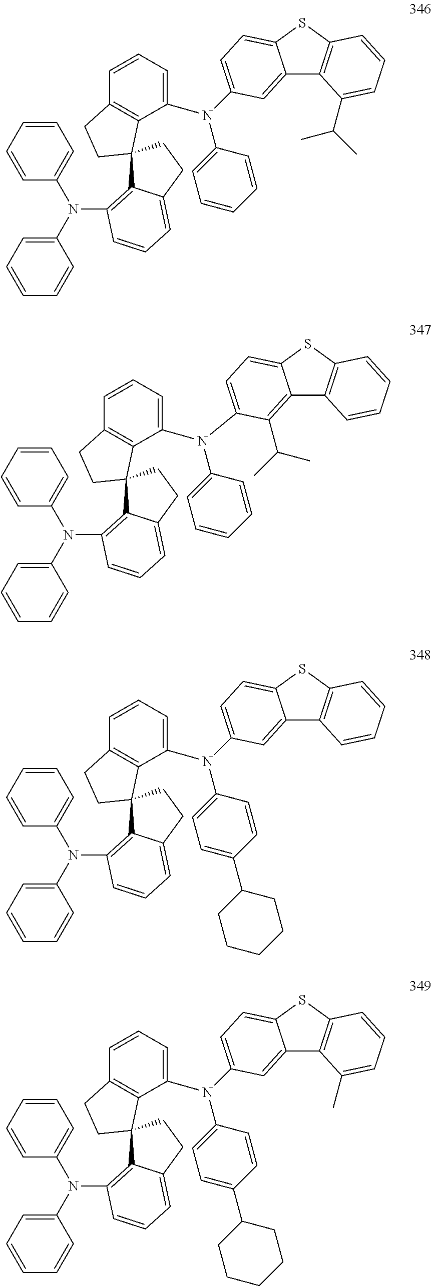 [SMILES: CC(C)c1cccc2sc3ccc(N(c4ccccc4)c4cccc5c4[C@]4(CCc6cccc(N(c7ccccc7)c7ccccc7)c64)CC5)cc3c12 ; CC(C)c1c(N(c2ccccc2)c2cccc3c2[C@]2(CCc4cccc(N(c5ccccc5)c5ccccc5)c42)CC3)ccc2sc3ccccc3c12 ; c1ccc(N(c2ccccc2)c2cccc3c2[C@@]2(CC3)CCc3cccc(N(c4ccc(C5CCCCC5)cc4)c4ccc5sc6ccccc6c5c4)c32)cc1 ; Cc1cccc2sc3ccc(N(c4ccc(C5CCCCC5)cc4)c4cccc5c4[C@]4(CCc6cccc(N(c7ccccc7)c7ccccc7)c64)CC5)cc3c12]

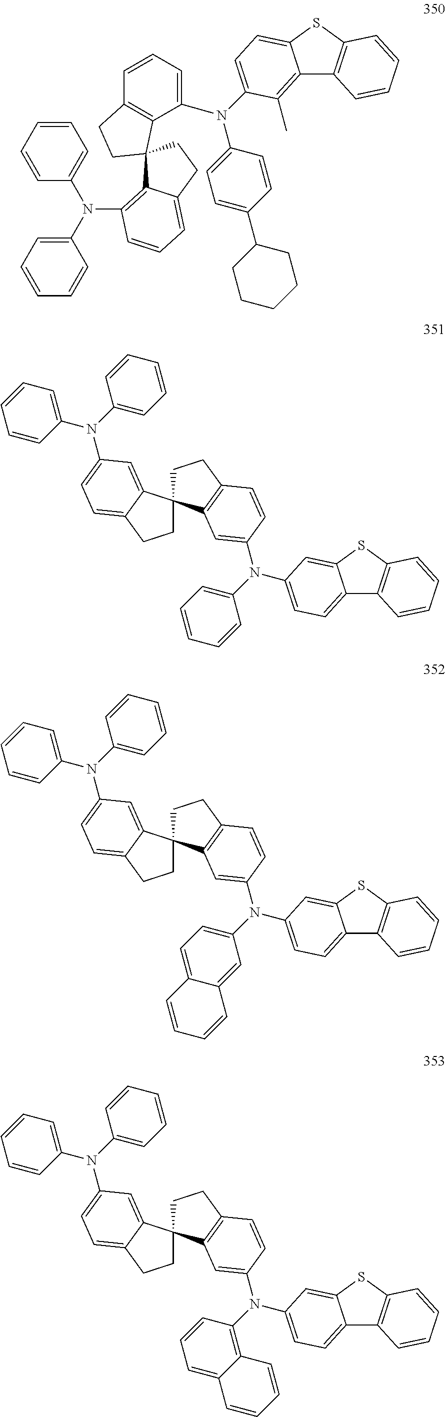 [SMILES: Cc1c(N(c2ccc(C3CCCCC3)cc2)c2cccc3c2[C@]2(CCc4cccc(N(c5ccccc5)c5ccccc5)c42)CC3)ccc2sc3ccccc3c12 ; c1ccc(N(c2ccccc2)c2ccc3c(c2)[C@@]2(CC3)CCc3ccc(N(c4ccccc4)c4ccc5c(c4)sc4ccccc45)cc32)cc1 ; c1ccc(N(c2ccccc2)c2ccc3c(c2)[C@@]2(CC3)CCc3ccc(N(c4ccc5ccccc5c4)c4ccc5c(c4)sc4ccccc45)cc32)cc1 ; c1ccc(N(c2ccccc2)c2ccc3c(c2)[C@@]2(CC3)CCc3ccc(N(c4ccc5c(c4)sc4ccccc45)c4cccc5ccccc45)cc32)cc1]

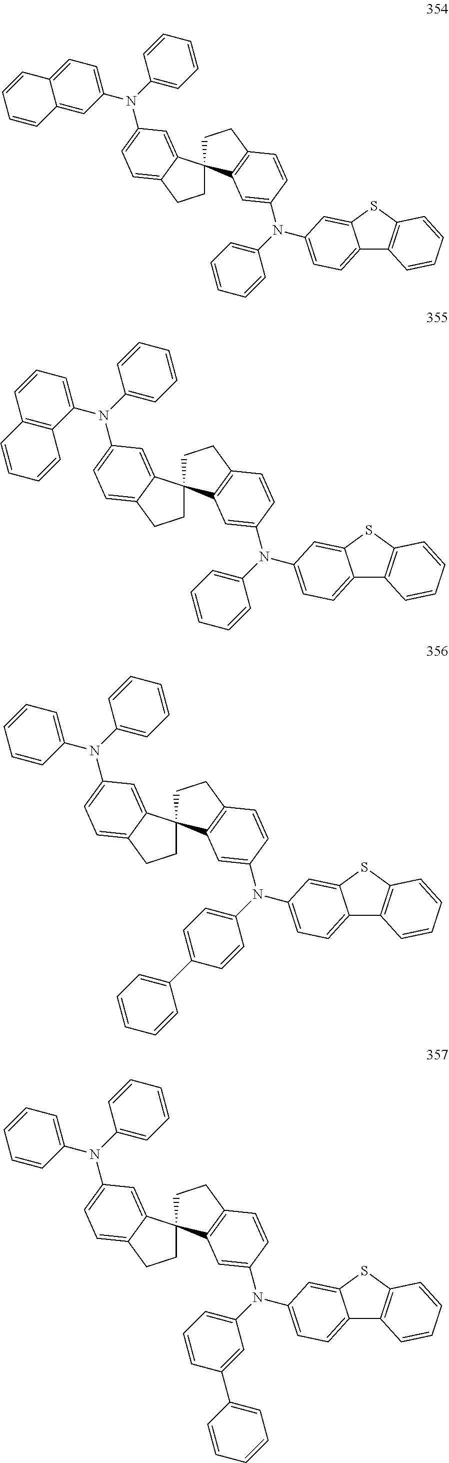 [SMILES: c1ccc(N(c2ccc3c(c2)[C@@]2(CC3)CCc3ccc(N(c4ccccc4)c4ccc5c(c4)sc4ccccc45)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)sc2ccccc23)cc1 ; c1ccc(-c2ccc(N(c3ccc4c(c3)[C@]3(CCc5ccc(N(c6ccccc6)c6ccccc6)cc53)CC4)c3ccc4c(c3)sc3ccccc34)cc2)cc1 ; c1ccc(-c2cccc(N(c3ccc4c(c3)[C@]3(CCc5ccc(N(c6ccccc6)c6ccccc6)cc53)CC4)c3ccc4c(c3)sc3ccccc34)c2)cc1]

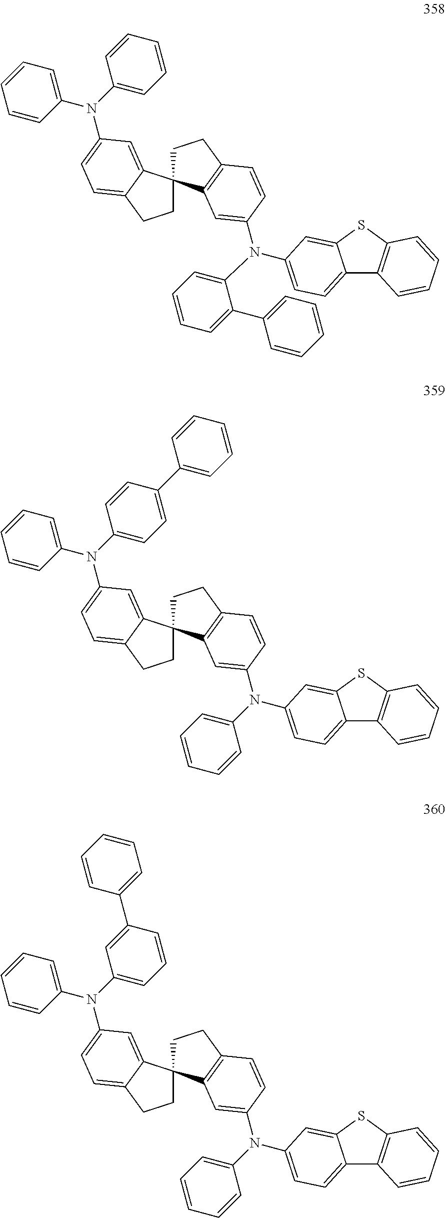 [SMILES: c1ccc(-c2ccccc2N(c2ccc3c(c2)[C@]2(CCc4ccc(N(c5ccccc5)c5ccccc5)cc42)CC3)c2ccc3c(c2)sc2ccccc23)cc1 ; c1ccc(-c2ccc(N(c3ccccc3)c3ccc4c(c3)[C@@]3(CC4)CCc4ccc(N(c5ccccc5)c5ccc6c(c5)sc5ccccc56)cc43)cc2)cc1 ; c1ccc(-c2cccc(N(c3ccccc3)c3ccc4c(c3)[C@@]3(CC4)CCc4ccc(N(c5ccccc5)c5ccc6c(c5)sc5ccccc56)cc43)c2)cc1]

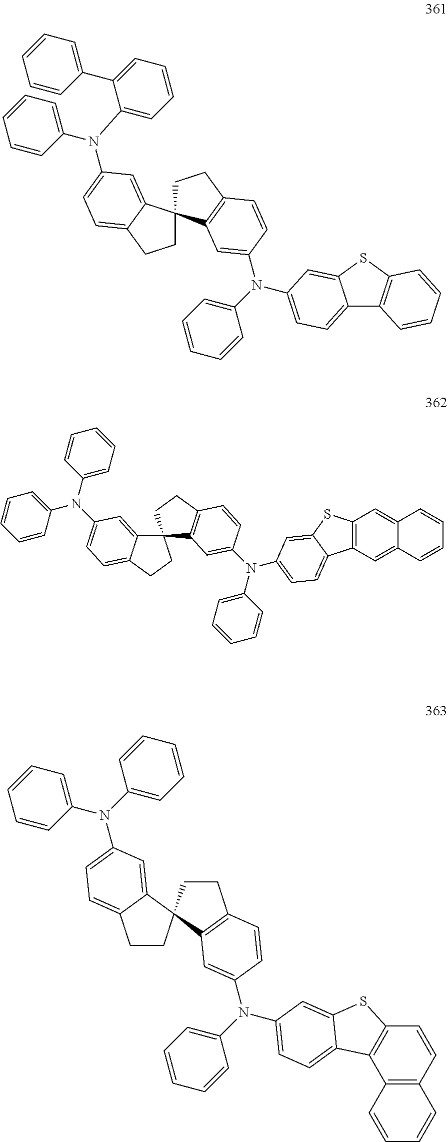 [SMILES: c1ccc(-c2ccccc2N(c2ccccc2)c2ccc3c(c2)[C@]2(CCc4ccc(N(c5ccccc5)c5ccc6c(c5)sc5ccccc56)cc42)CC3)cc1 ; c1ccc(N(c2ccccc2)c2ccc3c(c2)[C@@]2(CC3)CCc3ccc(N(c4ccccc4)c4ccc5c(c4)sc4cc6ccccc6cc45)cc32)cc1 ; c1ccc(N(c2ccccc2)c2ccc3c(c2)[C@@]2(CC3)CCc3ccc(N(c4ccccc4)c4ccc5c(c4)sc4ccc6ccccc6c45)cc32)cc1]

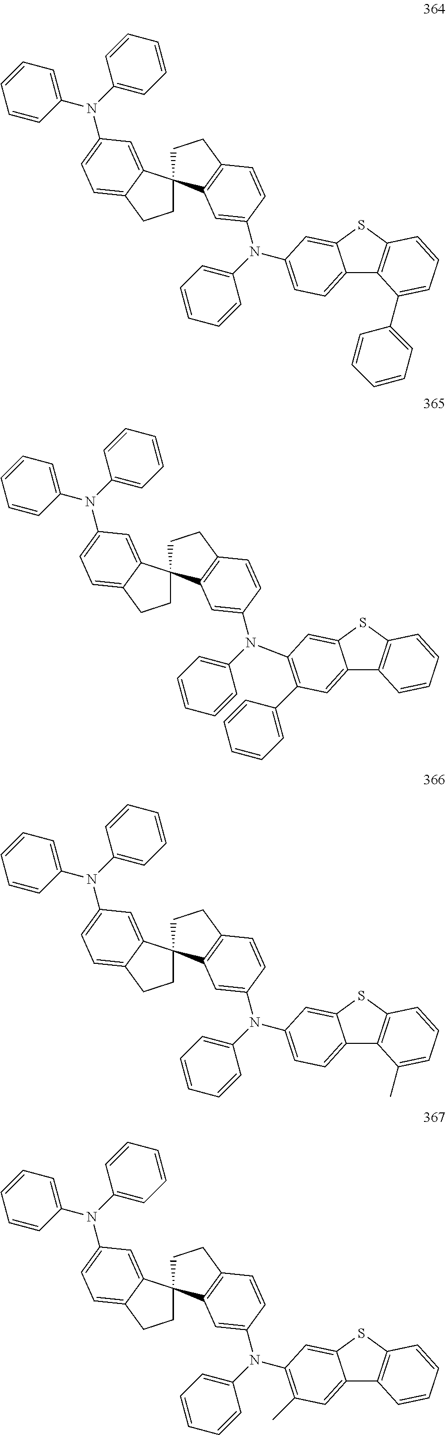 [SMILES: c1ccc(-c2cccc3sc4cc(N(c5ccccc5)c5ccc6c(c5)[C@]5(CCc7ccc(N(c8ccccc8)c8ccccc8)cc75)CC6)ccc4c23)cc1 ; c1ccc(-c2cc3c(cc2N(c2ccccc2)c2ccc4c(c2)[C@]2(CCc5ccc(N(c6ccccc6)c6ccccc6)cc52)CC4)sc2ccccc23)cc1 ; Cc1cccc2sc3cc(N(c4ccccc4)c4ccc5c(c4)[C@]4(CCc6ccc(N(c7ccccc7)c7ccccc7)cc64)CC5)ccc3c12 ; Cc1cc2c(cc1N(c1ccccc1)c1ccc3c(c1)[C@]1(CCc4ccc(N(c5ccccc5)c5ccccc5)cc41)CC3)sc1ccccc12]

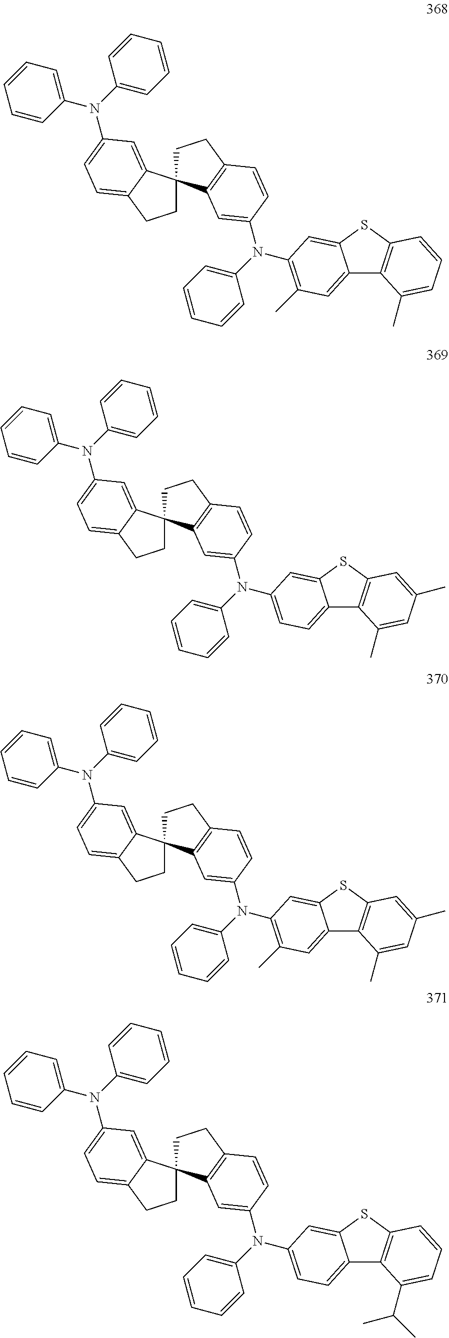 [SMILES: Cc1cc2c(cc1N(c1ccccc1)c1ccc3c(c1)[C@]1(CCc4ccc(N(c5ccccc5)c5ccccc5)cc41)CC3)sc1cccc(C)c12 ; Cc1cc(C)c2c(c1)sc1cc(N(c3ccccc3)c3ccc4c(c3)[C@]3(CCc5ccc(N(c6ccccc6)c6ccccc6)cc53)CC4)ccc12 ; Cc1cc(C)c2c(c1)sc1cc(N(c3ccccc3)c3ccc4c(c3)[C@]3(CCc5ccc(N(c6ccccc6)c6ccccc6)cc53)CC4)c(C)cc12 ; CC(C)c1cccc2sc3cc(N(c4ccccc4)c4ccc5c(c4)[C@]4(CCc6ccc(N(c7ccccc7)c7ccccc7)cc64)CC5)ccc3c12]

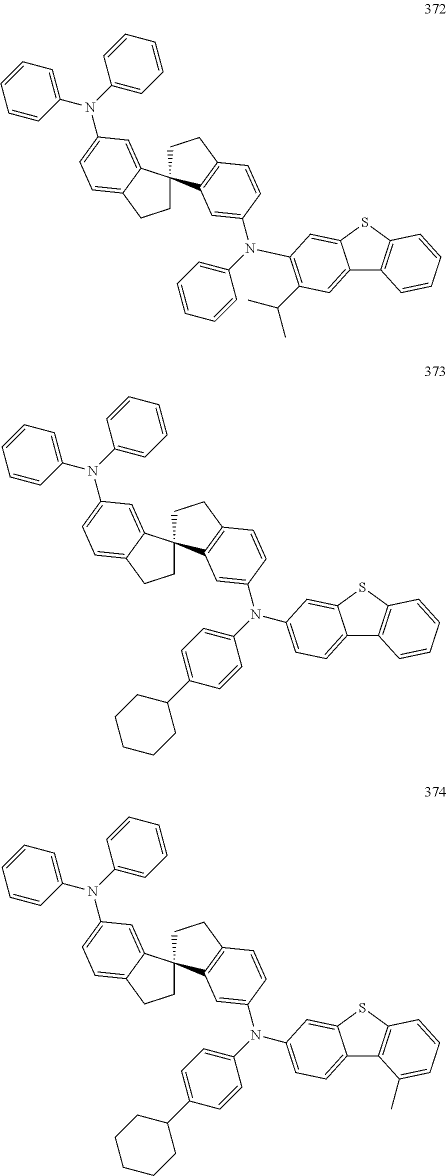 [SMILES: CC(C)c1cc2c(cc1N(c1ccccc1)c1ccc3c(c1)[C@]1(CCc4ccc(N(c5ccccc5)c5ccccc5)cc41)CC3)sc1ccccc12 ; c1ccc(N(c2ccccc2)c2ccc3c(c2)[C@@]2(CC3)CCc3ccc(N(c4ccc(C5CCCCC5)cc4)c4ccc5c(c4)sc4ccccc45)cc32)cc1 ; Cc1cccc2sc3cc(N(c4ccc(C5CCCCC5)cc4)c4ccc5c(c4)[C@]4(CCc6ccc(N(c7ccccc7)c7ccccc7)cc64)CC5)ccc3c12]

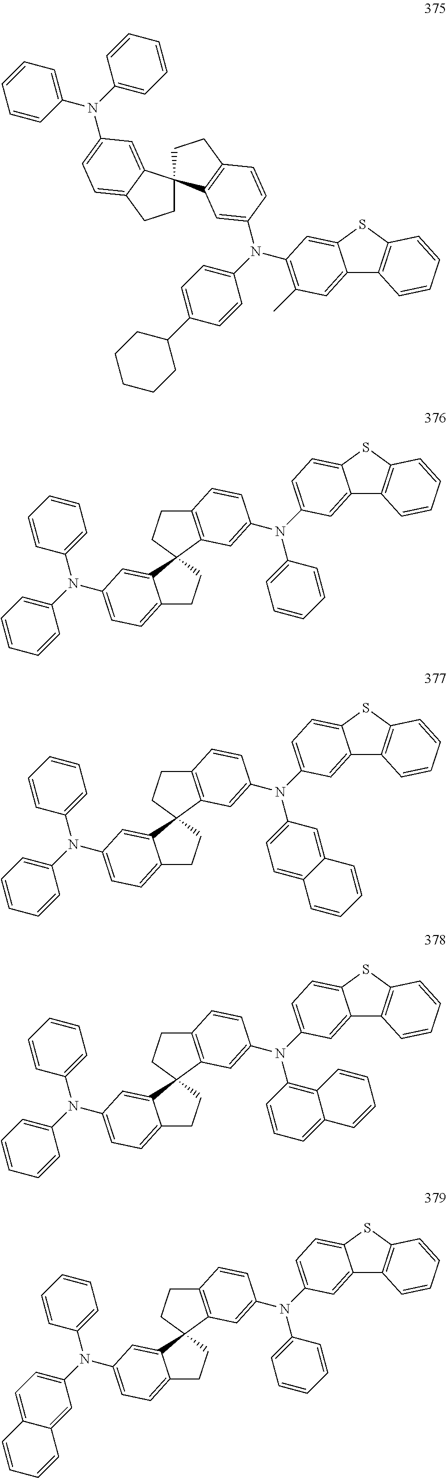 [SMILES: Cc1cc2c(cc1N(c1ccc(C3CCCCC3)cc1)c1ccc3c(c1)[C@]1(CCc4ccc(N(c5ccccc5)c5ccccc5)cc41)CC3)sc1ccccc12 ; c1ccc(N(c2ccccc2)c2ccc3c(c2)[C@@]2(CC3)CCc3ccc(N(c4ccccc4)c4ccc5sc6ccccc6c5c4)cc32)cc1 ; c1ccc(N(c2ccccc2)c2ccc3c(c2)[C@@]2(CC3)CCc3ccc(N(c4ccc5ccccc5c4)c4ccc5sc6ccccc6c5c4)cc32)cc1 ; c1ccc(N(c2ccccc2)c2ccc3c(c2)[C@@]2(CC3)CCc3ccc(N(c4ccc5sc6ccccc6c5c4)c4cccc5ccccc45)cc32)cc1 ; c1ccc(N(c2ccc3c(c2)[C@@]2(CC3)CCc3ccc(N(c4ccccc4)c4ccc5sc6ccccc6c5c4)cc32)c2ccc3ccccc3c2)cc1]

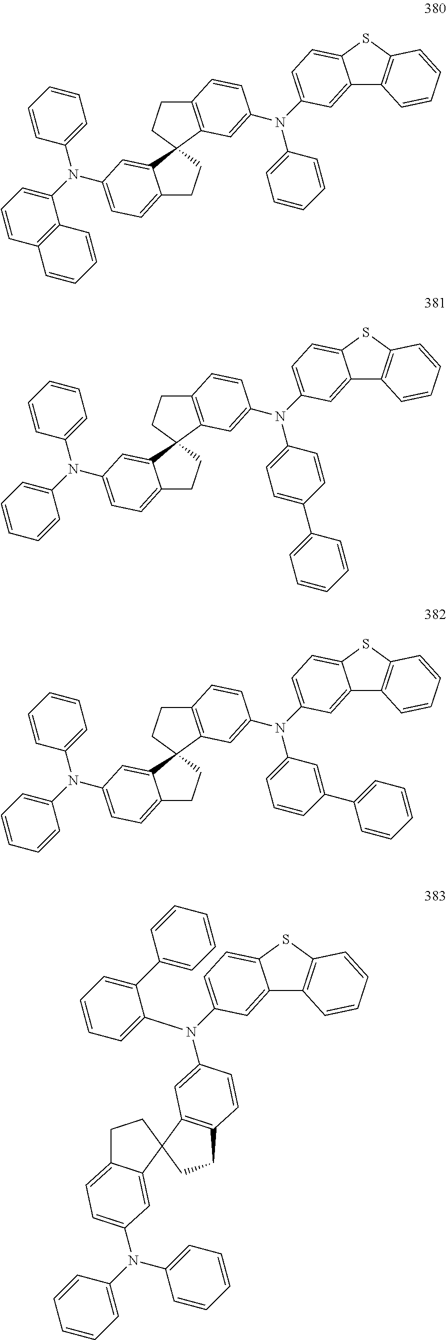

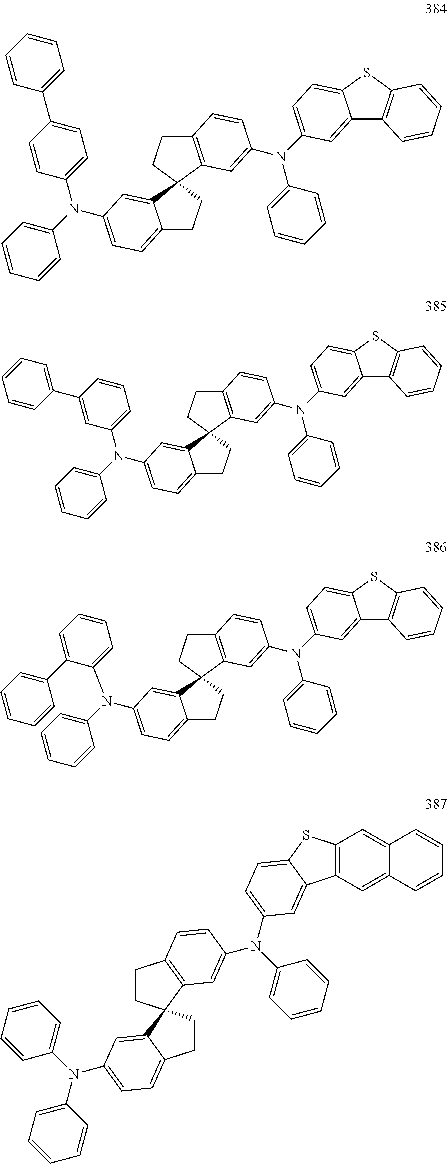 [SMILES: c1ccc(-c2ccc(N(c3ccccc3)c3ccc4c(c3)[C@@]3(CC4)CCc4ccc(N(c5ccccc5)c5ccc6sc7ccccc7c6c5)cc43)cc2)cc1 ; c1ccc(-c2cccc(N(c3ccccc3)c3ccc4c(c3)[C@@]3(CC4)CCc4ccc(N(c5ccccc5)c5ccc6sc7ccccc7c6c5)cc43)c2)cc1 ; c1ccc(-c2ccccc2N(c2ccccc2)c2ccc3c(c2)[C@]2(CCc4ccc(N(c5ccccc5)c5ccc6sc7ccccc7c6c5)cc42)CC3)cc1 ; c1ccc(N(c2ccccc2)c2ccc3c(c2)[C@@]2(CC3)CCc3ccc(N(c4ccccc4)c4ccc5sc6cc7ccccc7cc6c5c4)cc32)cc1]

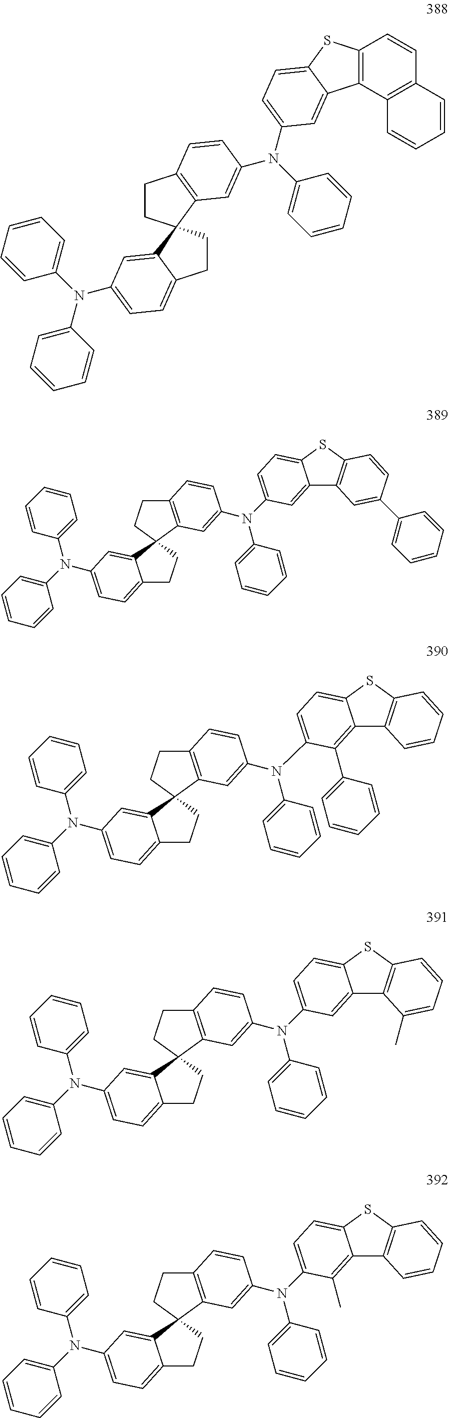 [SMILES: c1ccc(N(c2ccccc2)c2ccc3c(c2)[C@@]2(CC3)CCc3ccc(N(c4ccccc4)c4ccc5sc6ccc7ccccc7c6c5c4)cc32)cc1 ; c1ccc(-c2ccc3sc4ccc(N(c5ccccc5)c5ccc6c(c5)[C@]5(CCc7ccc(N(c8ccccc8)c8ccccc8)cc75)CC6)cc4c3c2)cc1 ; c1ccc(-c2c(N(c3ccccc3)c3ccc4c(c3)[C@]3(CCc5ccc(N(c6ccccc6)c6ccccc6)cc53)CC4)ccc3sc4ccccc4c23)cc1 ; Cc1cccc2sc3ccc(N(c4ccccc4)c4ccc5c(c4)[C@]4(CCc6ccc(N(c7ccccc7)c7ccccc7)cc64)CC5)cc3c12 ; Cc1c(N(c2ccccc2)c2ccc3c(c2)[C@]2(CCc4ccc(N(c5ccccc5)c5ccccc5)cc42)CC3)ccc2sc3ccccc3c12]

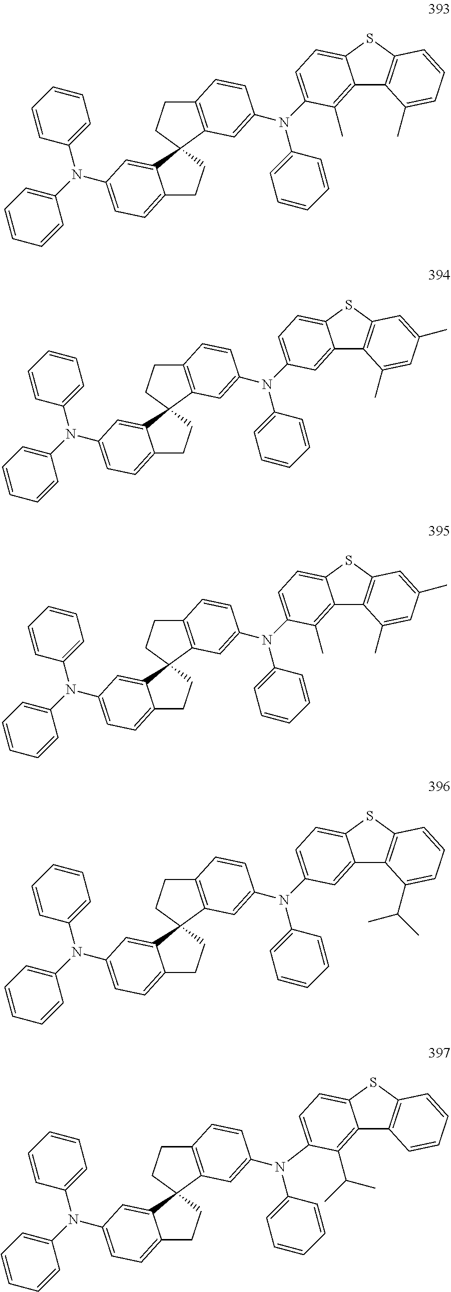 [SMILES: Cc1cccc2sc3ccc(N(c4ccccc4)c4ccc5c(c4)[C@]4(CCc6ccc(N(c7ccccc7)c7ccccc7)cc64)CC5)c(C)c3c12 ; Cc1cc(C)c2c(c1)sc1ccc(N(c3ccccc3)c3ccc4c(c3)[C@]3(CCc5ccc(N(c6ccccc6)c6ccccc6)cc53)CC4)cc12 ; Cc1cc(C)c2c(c1)sc1ccc(N(c3ccccc3)c3ccc4c(c3)[C@]3(CCc5ccc(N(c6ccccc6)c6ccccc6)cc53)CC4)c(C)c12 ; CC(C)c1cccc2sc3ccc(N(c4ccccc4)c4ccc5c(c4)[C@]4(CCc6ccc(N(c7ccccc7)c7ccccc7)cc64)CC5)cc3c12 ; CC(C)c1c(N(c2ccccc2)c2ccc3c(c2)[C@]2(CCc4ccc(N(c5ccccc5)c5ccccc5)cc42)CC3)ccc2sc3ccccc3c12]

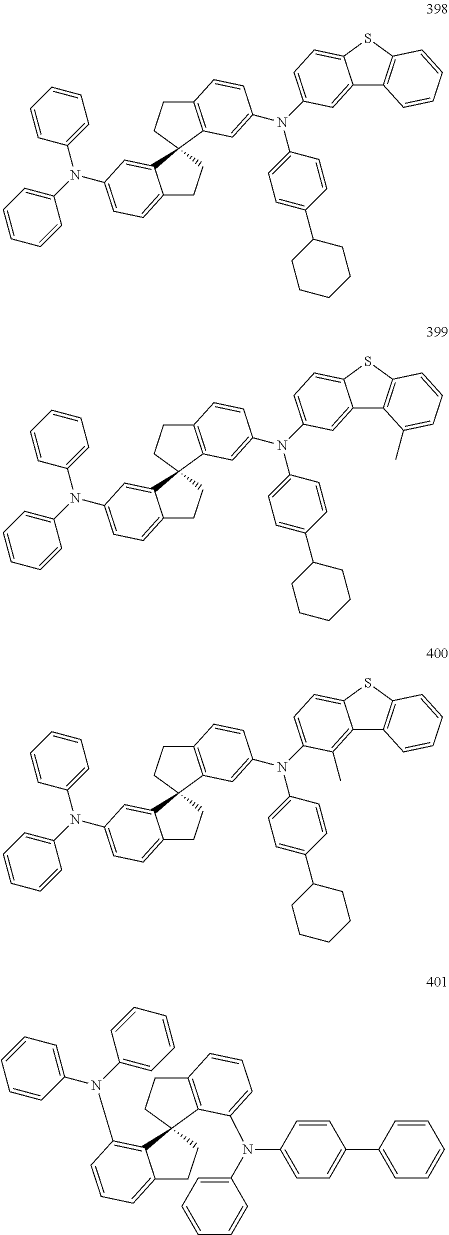 [SMILES: c1ccc(N(c2ccccc2)c2ccc3c(c2)[C@@]2(CC3)CCc3ccc(N(c4ccc(C5CCCCC5)cc4)c4ccc5sc6ccccc6c5c4)cc32)cc1 ; Cc1cccc2sc3ccc(N(c4ccc(C5CCCCC5)cc4)c4ccc5c(c4)[C@]4(CCc6ccc(N(c7ccccc7)c7ccccc7)cc64)CC5)cc3c12 ; Cc1c(N(c2ccc(C3CCCCC3)cc2)c2ccc3c(c2)[C@]2(CCc4ccc(N(c5ccccc5)c5ccccc5)cc42)CC3)ccc2sc3ccccc3c12 ; c1ccc(-c2ccc(N(c3ccccc3)c3cccc4c3[C@]3(CCc5cccc(N(c6ccccc6)c6ccccc6)c53)CC4)cc2)cc1]

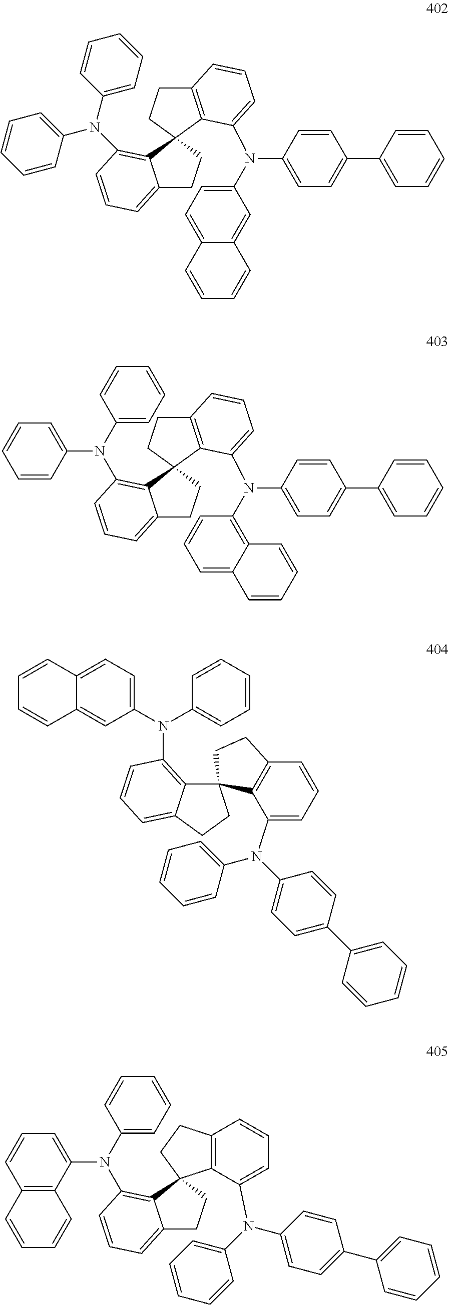 [SMILES: c1ccc(-c2ccc(N(c3ccc4ccccc4c3)c3cccc4c3[C@]3(CCc5cccc(N(c6ccccc6)c6ccccc6)c53)CC4)cc2)cc1 ; c1ccc(-c2ccc(N(c3cccc4c3[C@]3(CCc5cccc(N(c6ccccc6)c6ccccc6)c53)CC4)c3cccc4ccccc34)cc2)cc1 ; c1ccc(-c2ccc(N(c3ccccc3)c3cccc4c3[C@@]3(CC4)CCc4cccc(N(c5ccccc5)c5ccc6ccccc6c5)c43)cc2)cc1 ; c1ccc(-c2ccc(N(c3ccccc3)c3cccc4c3[C@@]3(CC4)CCc4cccc(N(c5ccccc5)c5cccc6ccccc56)c43)cc2)cc1]

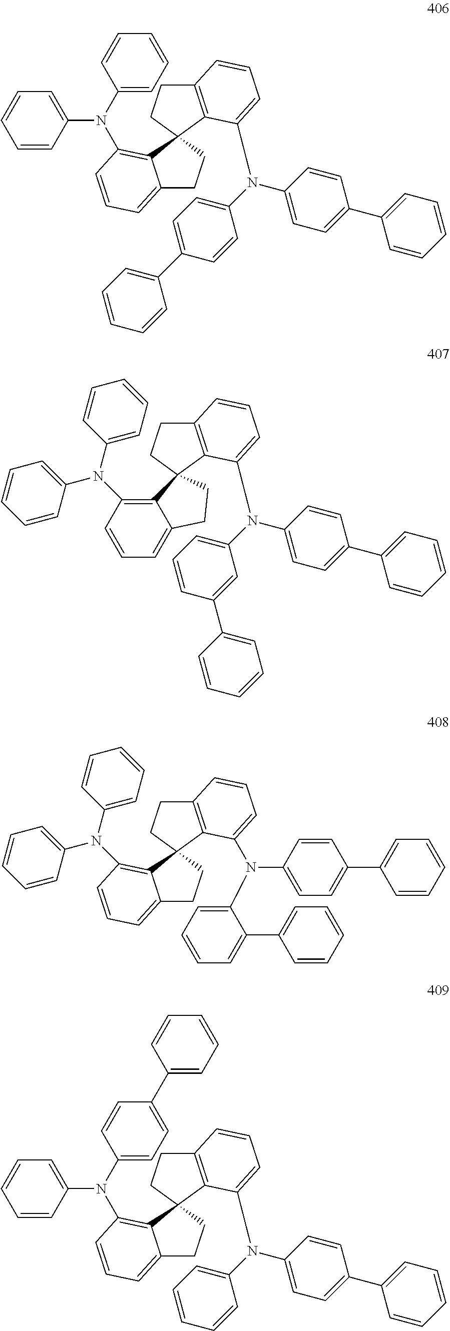

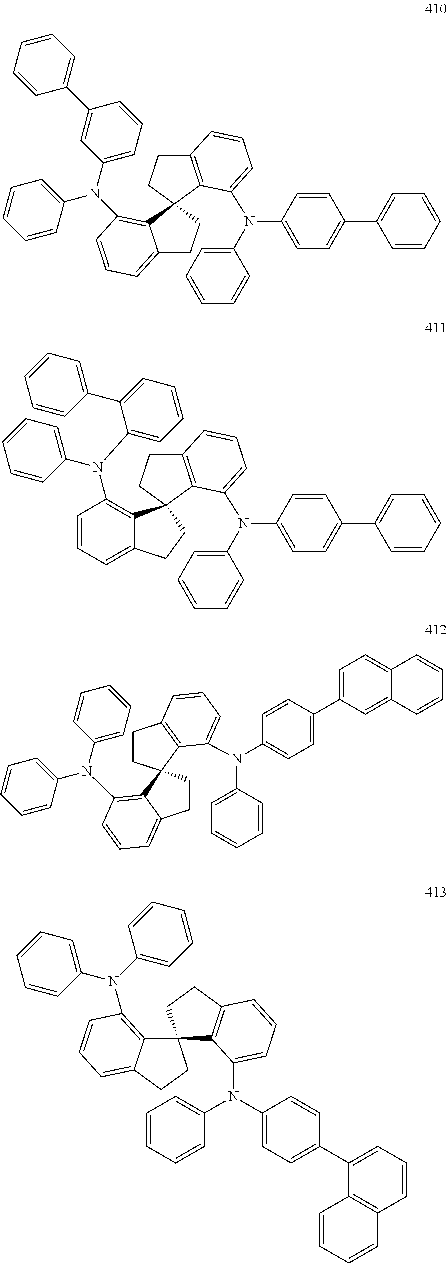 [SMILES: c1ccc(-c2ccc(N(c3ccccc3)c3cccc4c3[C@@]3(CC4)CCc4cccc(N(c5ccccc5)c5cccc(-c6ccccc6)c5)c43)cc2)cc1 ; c1ccc(-c2ccc(N(c3ccccc3)c3cccc4c3[C@@]3(CC4)CCc4cccc(N(c5ccccc5)c5ccccc5-c5ccccc5)c43)cc2)cc1 ; c1ccc(N(c2ccccc2)c2cccc3c2[C@@]2(CC3)CCc3cccc(N(c4ccccc4)c4ccc(-c5ccc6ccccc6c5)cc4)c32)cc1 ; c1ccc(N(c2ccccc2)c2cccc3c2[C@@]2(CC3)CCc3cccc(N(c4ccccc4)c4ccc(-c5cccc6ccccc56)cc4)c32)cc1]

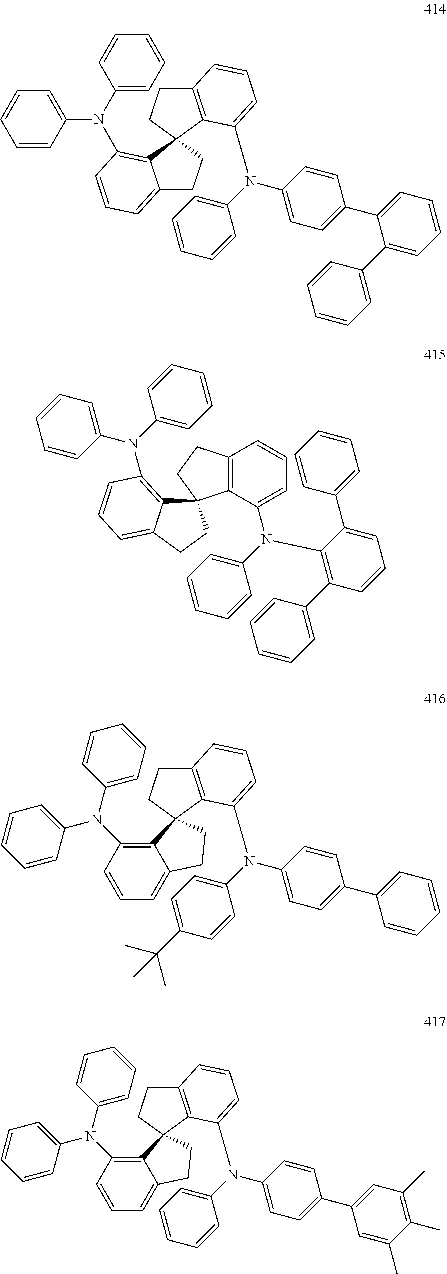 [SMILES: c1ccc(-c2ccccc2-c2ccc(N(c3ccccc3)c3cccc4c3[C@]3(CCc5cccc(N(c6ccccc6)c6ccccc6)c53)CC4)cc2)cc1 ; c1ccc(-c2cccc(-c3ccccc3)c2N(c2ccccc2)c2cccc3c2[C@]2(CCc4cccc(N(c5ccccc5)c5ccccc5)c42)CC3)cc1 ; CC(C)(C)c1ccc(N(c2ccc(-c3ccccc3)cc2)c2cccc3c2[C@]2(CCc4cccc(N(c5ccccc5)c5ccccc5)c42)CC3)cc1 ; Cc1cc(-c2ccc(N(c3ccccc3)c3cccc4c3[C@]3(CCc5cccc(N(c6ccccc6)c6ccccc6)c53)CC4)cc2)cc(C)c1C]

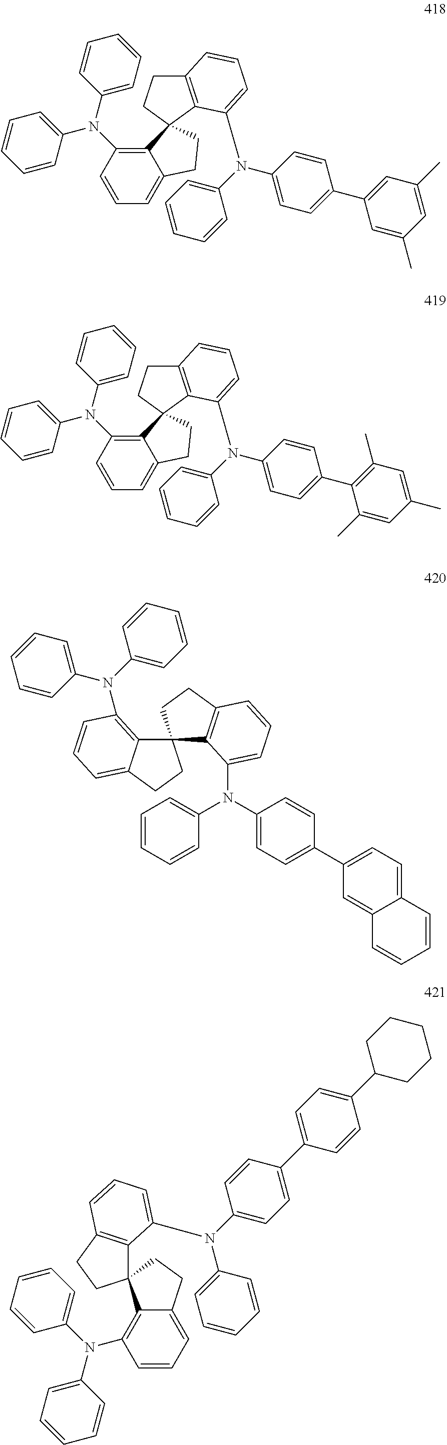 [SMILES: Cc1cc(C)cc(-c2ccc(N(c3ccccc3)c3cccc4c3[C@]3(CCc5cccc(N(c6ccccc6)c6ccccc6)c53)CC4)cc2)c1 ; Cc1cc(C)c(-c2ccc(N(c3ccccc3)c3cccc4c3[C@]3(CCc5cccc(N(c6ccccc6)c6ccccc6)c53)CC4)cc2)c(C)c1 ; c1ccc(N(c2ccccc2)c2cccc3c2[C@@]2(CC3)CCc3cccc(N(c4ccccc4)c4ccc(-c5ccc6ccccc6c5)cc4)c32)cc1 ; c1ccc(N(c2ccccc2)c2cccc3c2[C@@]2(CC3)CCc3cccc(N(c4ccccc4)c4ccc(-c5ccc(C6CCCCC6)cc5)cc4)c32)cc1]

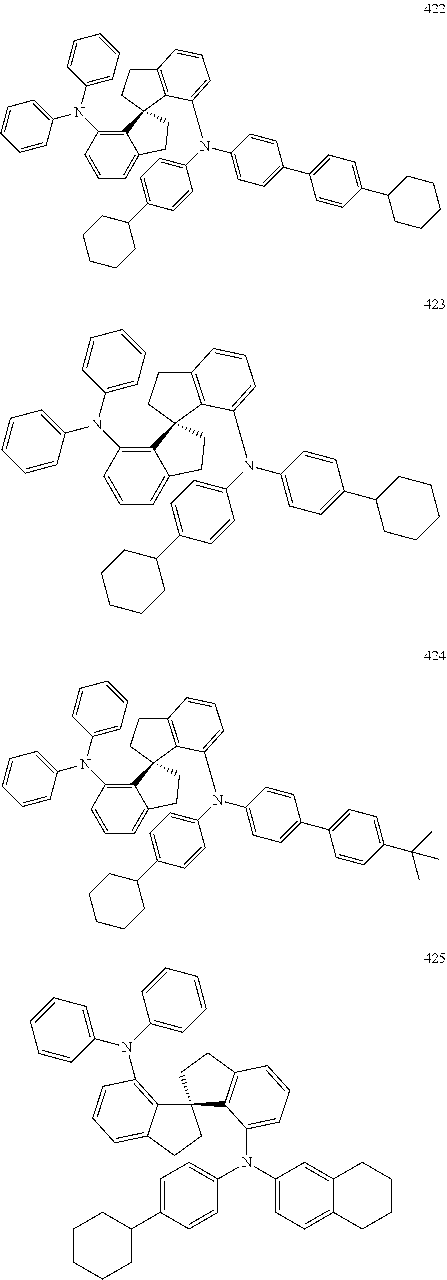 [SMILES: c1ccc(N(c2ccccc2)c2cccc3c2[C@@]2(CC3)CCc3cccc(N(c4ccc(-c5ccc(C6CCCCC6)cc5)cc4)c4ccc(C5CCCCC5)cc4)c32)cc1 ; c1ccc(N(c2ccccc2)c2cccc3c2[C@@]2(CC3)CCc3cccc(N(c4ccc(C5CCCCC5)cc4)c4ccc(C5CCCCC5)cc4)c32)cc1 ; CC(C)(C)c1ccc(-c2ccc(N(c3ccc(C4CCCCC4)cc3)c3cccc4c3[C@]3(CCc5cccc(N(c6ccccc6)c6ccccc6)c53)CC4)cc2)cc1 ; c1ccc(N(c2ccccc2)c2cccc3c2[C@@]2(CC3)CCc3cccc(N(c4ccc(C5CCCCC5)cc4)c4ccc5c(c4)CCCC5)c32)cc1]

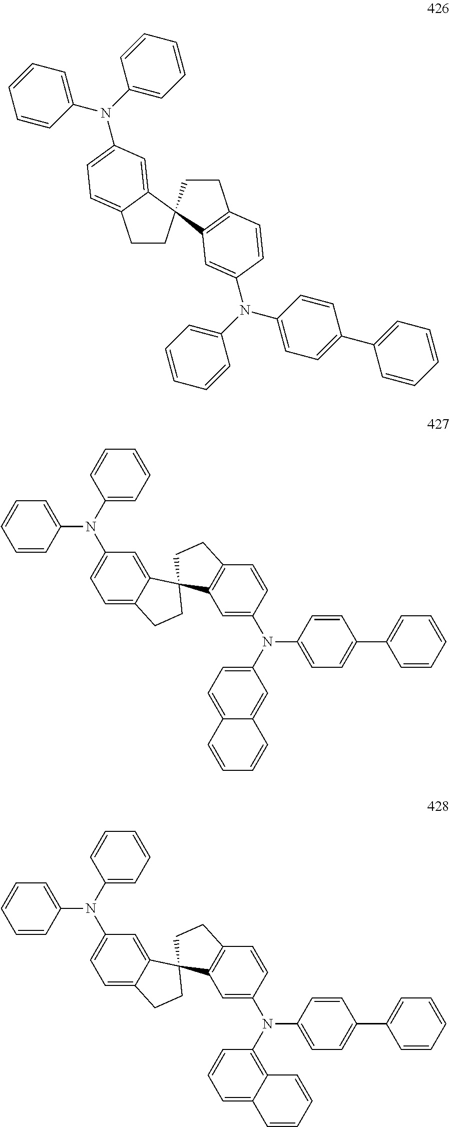 [SMILES: c1ccc(-c2ccc(N(c3ccccc3)c3ccc4c(c3)[C@]3(CCc5ccc(N(c6ccccc6)c6ccccc6)cc53)CC4)cc2)cc1 ; c1ccc(-c2ccc(N(c3ccc4c(c3)[C@]3(CCc5ccc(N(c6ccccc6)c6ccccc6)cc53)CC4)c3ccc4ccccc4c3)cc2)cc1 ; c1ccc(-c2ccc(N(c3ccc4c(c3)[C@]3(CCc5ccc(N(c6ccccc6)c6ccccc6)cc53)CC4)c3cccc4ccccc34)cc2)cc1]

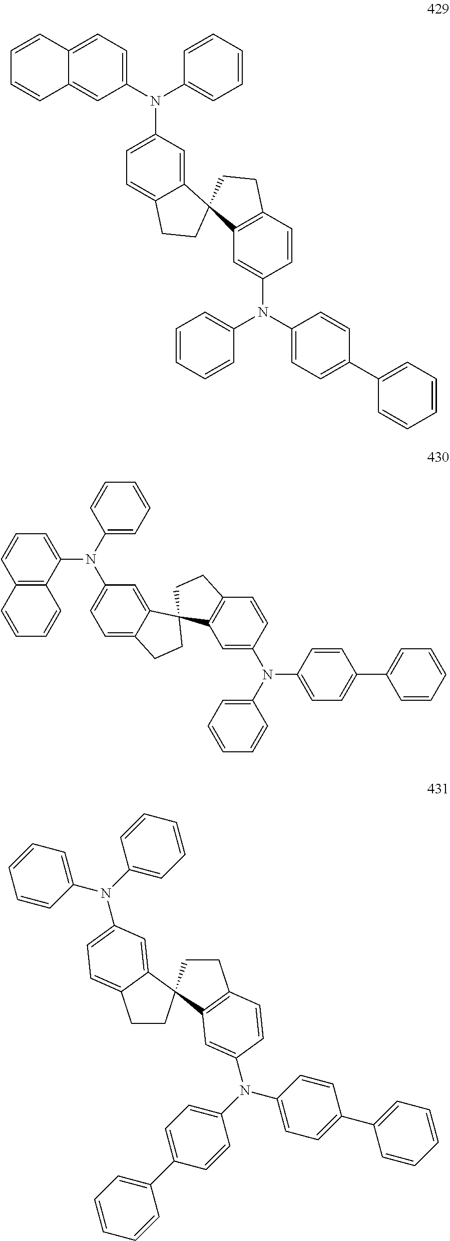 [SMILES: c1ccc(-c2ccc(N(c3ccccc3)c3ccc4c(c3)[C@@]3(CC4)CCc4ccc(N(c5ccccc5)c5ccc6ccccc6c5)cc43)cc2)cc1 ; c1ccc(-c2ccc(N(c3ccccc3)c3ccc4c(c3)[C@@]3(CC4)CCc4ccc(N(c5ccccc5)c5cccc6ccccc56)cc43)cc2)cc1 ; c1ccc(-c2ccc(N(c3ccc(-c4ccccc4)cc3)c3ccc4c(c3)[C@]3(CCc5ccc(N(c6ccccc6)c6ccccc6)cc53)CC4)cc2)cc1]

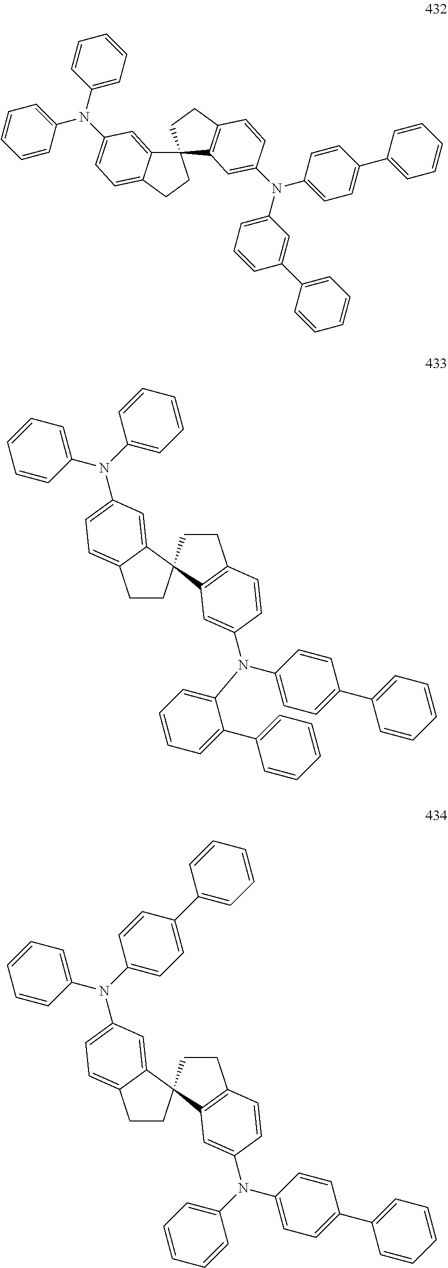

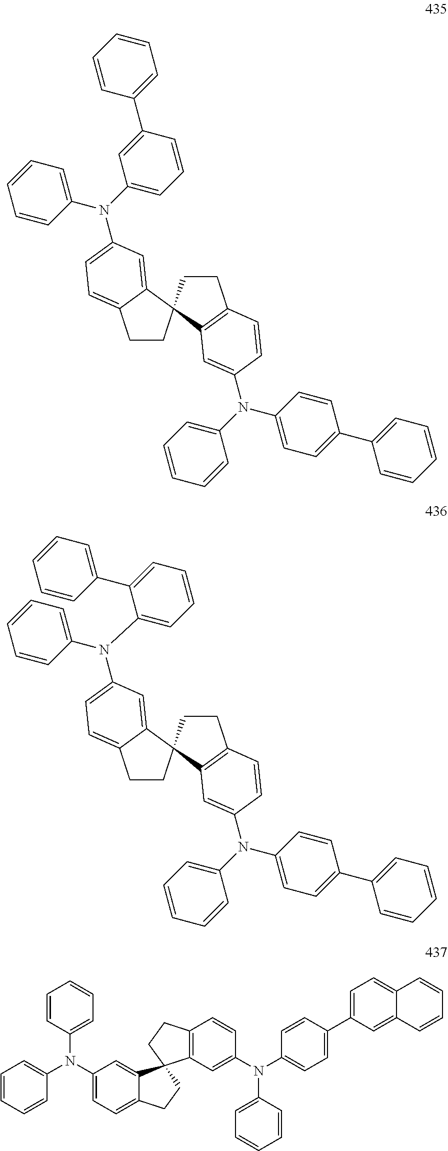 [SMILES: c1ccc(-c2ccc(N(c3ccccc3)c3ccc4c(c3)[C@@]3(CC4)CCc4ccc(N(c5ccccc5)c5cccc(-c6ccccc6)c5)cc43)cc2)cc1 ; c1ccc(-c2ccc(N(c3ccccc3)c3ccc4c(c3)[C@@]3(CC4)CCc4ccc(N(c5ccccc5)c5ccccc5-c5ccccc5)cc43)cc2)cc1 ; c1ccc(N(c2ccccc2)c2ccc3c(c2)[C@@]2(CC3)CCc3ccc(N(c4ccccc4)c4ccc(-c5ccc6ccccc6c5)cc4)cc32)cc1]

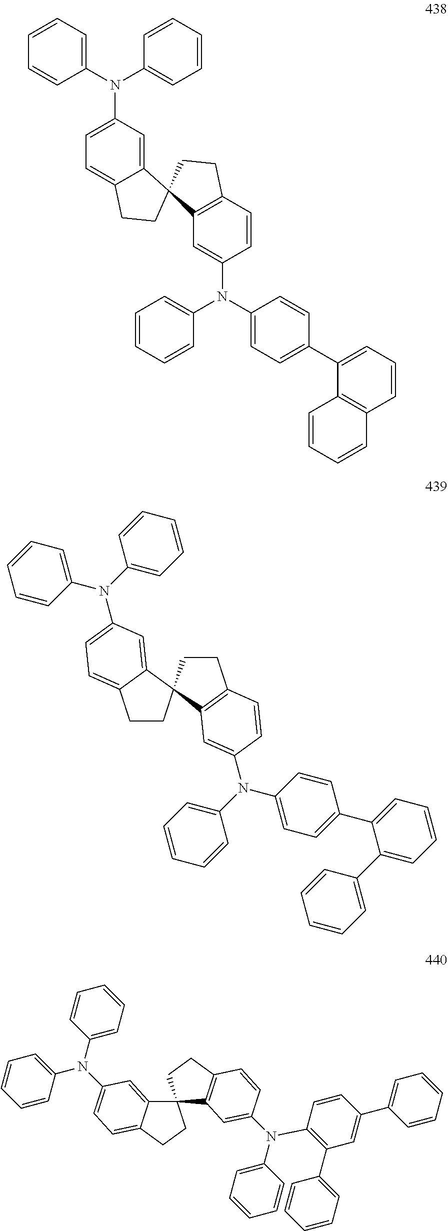 [SMILES: c1ccc(N(c2ccccc2)c2ccc3c(c2)[C@@]2(CC3)CCc3ccc(N(c4ccccc4)c4ccc(-c5cccc6ccccc56)cc4)cc32)cc1 ; c1ccc(-c2ccccc2-c2ccc(N(c3ccccc3)c3ccc4c(c3)[C@]3(CCc5ccc(N(c6ccccc6)c6ccccc6)cc53)CC4)cc2)cc1 ; c1ccc(-c2ccc(N(c3ccccc3)c3ccc4c(c3)[C@]3(CCc5ccc(N(c6ccccc6)c6ccccc6)cc53)CC4)c(-c3ccccc3)c2)cc1]

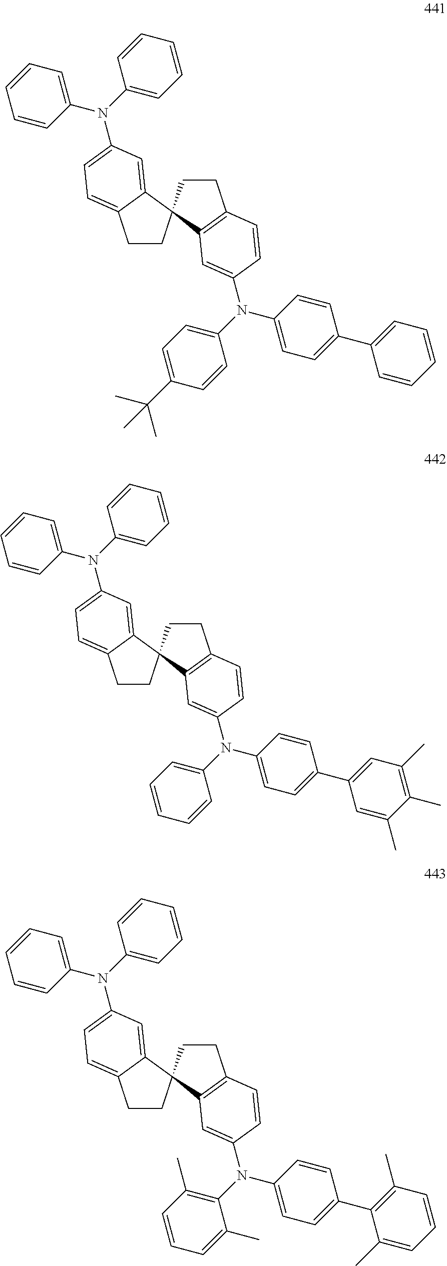 [SMILES: CC(C)(C)c1ccc(N(c2ccc(-c3ccccc3)cc2)c2ccc3c(c2)[C@]2(CCc4ccc(N(c5ccccc5)c5ccccc5)cc42)CC3)cc1 ; Cc1cc(-c2ccc(N(c3ccccc3)c3ccc4c(c3)[C@]3(CCc5ccc(N(c6ccccc6)c6ccccc6)cc53)CC4)cc2)cc(C)c1C ; Cc1cccc(C)c1-c1ccc(N(c2ccc3c(c2)[C@]2(CCc4ccc(N(c5ccccc5)c5ccccc5)cc42)CC3)c2c(C)cccc2C)cc1]

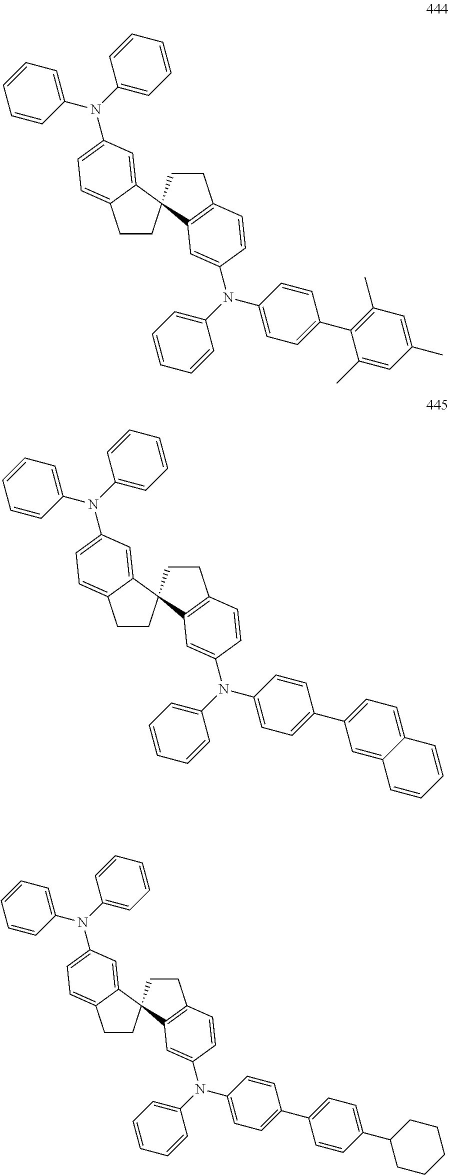 [SMILES: Cc1cc(C)c(-c2ccc(N(c3ccccc3)c3ccc4c(c3)[C@]3(CCc5ccc(N(c6ccccc6)c6ccccc6)cc53)CC4)cc2)c(C)c1 ; c1ccc(N(c2ccccc2)c2ccc3c(c2)[C@@]2(CC3)CCc3ccc(N(c4ccccc4)c4ccc(-c5ccc6ccccc6c5)cc4)cc32)cc1 ; c1ccc(N(c2ccccc2)c2ccc3c(c2)[C@@]2(CC3)CCc3ccc(N(c4ccccc4)c4ccc(-c5ccc(C6CCCCC6)cc5)cc4)cc32)cc1]

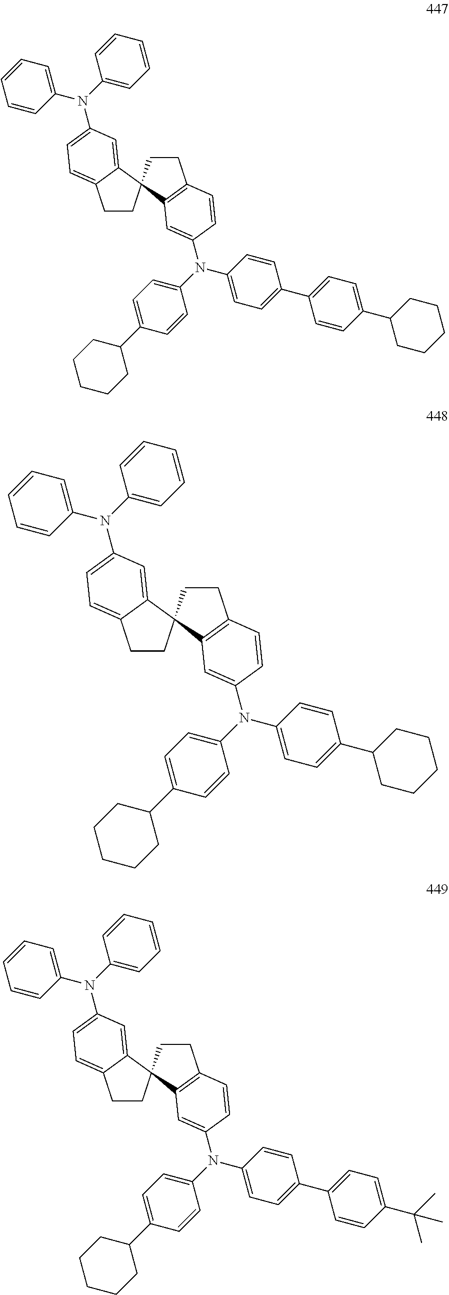 [SMILES: c1ccc(N(c2ccccc2)c2ccc3c(c2)[C@@]2(CC3)CCc3ccc(N(c4ccc(-c5ccc(C6CCCCC6)cc5)cc4)c4ccc(C5CCCCC5)cc4)cc32)cc1 ; c1ccc(N(c2ccccc2)c2ccc3c(c2)[C@@]2(CC3)CCc3ccc(N(c4ccc(C5CCCCC5)cc4)c4ccc(C5CCCCC5)cc4)cc32)cc1 ; CC(C)(C)c1ccc(-c2ccc(N(c3ccc(C4CCCCC4)cc3)c3ccc4c(c3)[C@]3(CCc5ccc(N(c6ccccc6)c6ccccc6)cc53)CC4)cc2)cc1]

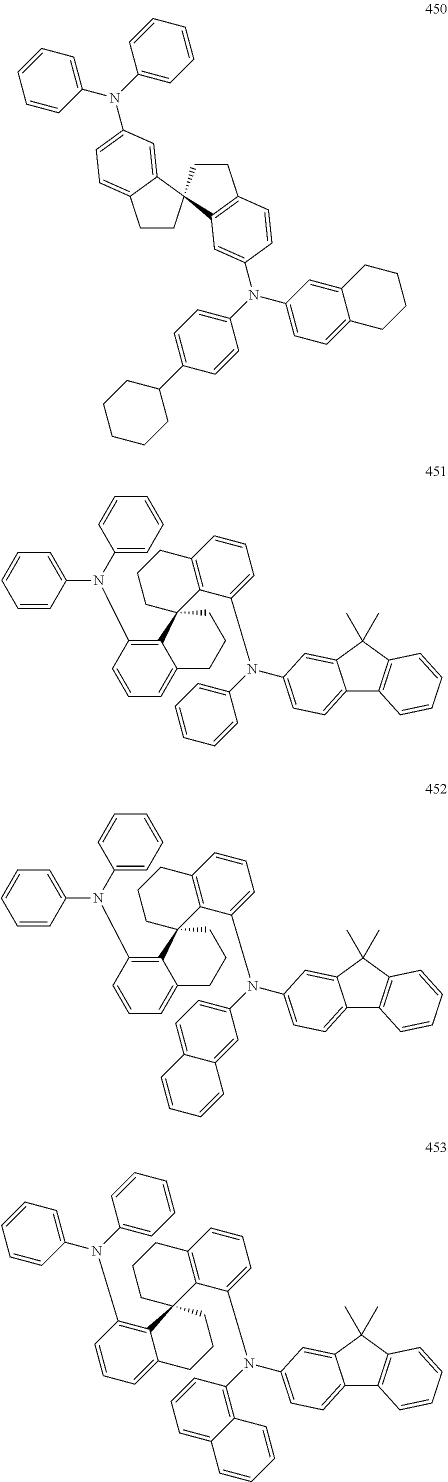 [SMILES: c1ccc(N(c2ccccc2)c2ccc3c(c2)[C@@]2(CC3)CCc3ccc(N(c4ccc(C5CCCCC5)cc4)c4ccc5c(c4)CCCC5)cc32)cc1 ; CC1(C)c2ccccc2-c2ccc(N(c3ccccc3)c3cccc4c3[C@]3(CCCc5cccc(N(c6ccccc6)c6ccccc6)c53)CCC4)cc21 ; CC1(C)c2ccccc2-c2ccc(N(c3ccc4ccccc4c3)c3cccc4c3[C@]3(CCCc5cccc(N(c6ccccc6)c6ccccc6)c53)CCC4)cc21 ; CC1(C)c2ccccc2-c2ccc(N(c3cccc4c3[C@]3(CCCc5cccc(N(c6ccccc6)c6ccccc6)c53)CCC4)c3cccc4ccccc34)cc21]

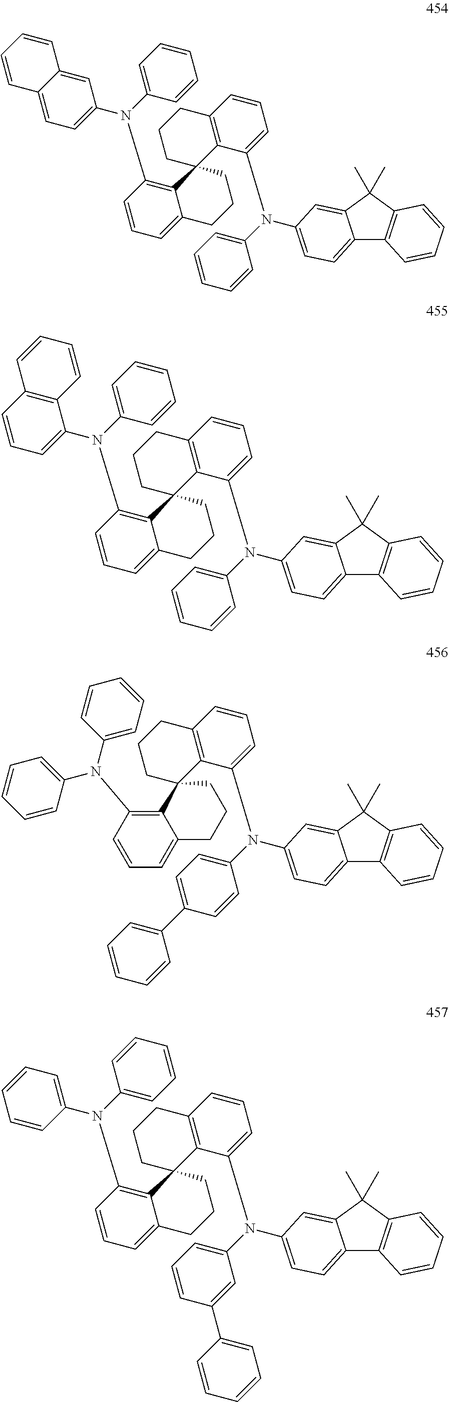 [SMILES: CC1(C)c2ccccc2-c2ccc(N(c3ccccc3)c3cccc4c3[C@@]3(CCC4)CCCc4cccc(N(c5ccccc5)c5ccc6ccccc6c5)c43)cc21 ; CC1(C)c2ccccc2-c2ccc(N(c3ccccc3)c3cccc4c3[C@@]3(CCC4)CCCc4cccc(N(c5ccccc5)c5cccc6ccccc56)c43)cc21 ; CC1(C)c2ccccc2-c2ccc(N(c3ccc(-c4ccccc4)cc3)c3cccc4c3[C@]3(CCCc5cccc(N(c6ccccc6)c6ccccc6)c53)CCC4)cc21 ; CC1(C)c2ccccc2-c2ccc(N(c3cccc(-c4ccccc4)c3)c3cccc4c3[C@]3(CCCc5cccc(N(c6ccccc6)c6ccccc6)c53)CCC4)cc21]

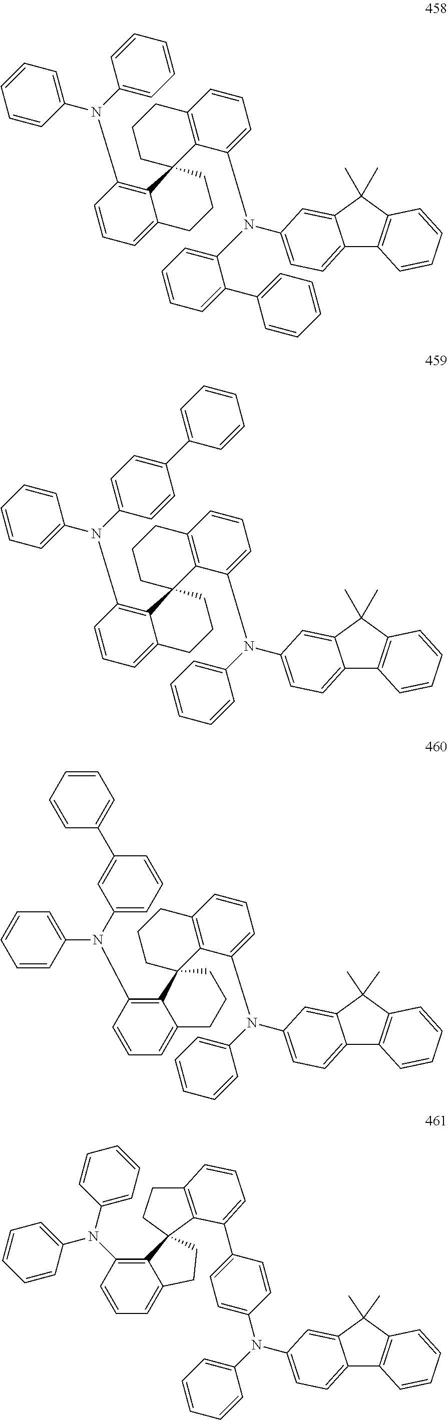 [SMILES: CC1(C)c2ccccc2-c2ccc(N(c3ccccc3-c3ccccc3)c3cccc4c3[C@]3(CCCc5cccc(N(c6ccccc6)c6ccccc6)c53)CCC4)cc21 ; CC1(C)c2ccccc2-c2ccc(N(c3ccccc3)c3cccc4c3[C@]3(CCCc5cccc(N(c6ccccc6)c6ccc(-c7ccccc7)cc6)c53)CCC4)cc21 ; CC1(C)c2ccccc2-c2ccc(N(c3ccccc3)c3cccc4c3[C@]3(CCCc5cccc(N(c6ccccc6)c6cccc(-c7ccccc7)c6)c53)CCC4)cc21 ; CC1(C)c2ccccc2-c2ccc(N(c3ccccc3)c3ccc(-c4cccc5c4[C@@]4(CC5)CCc5cccc(N(c6ccccc6)c6ccccc6)c54)cc3)cc21]

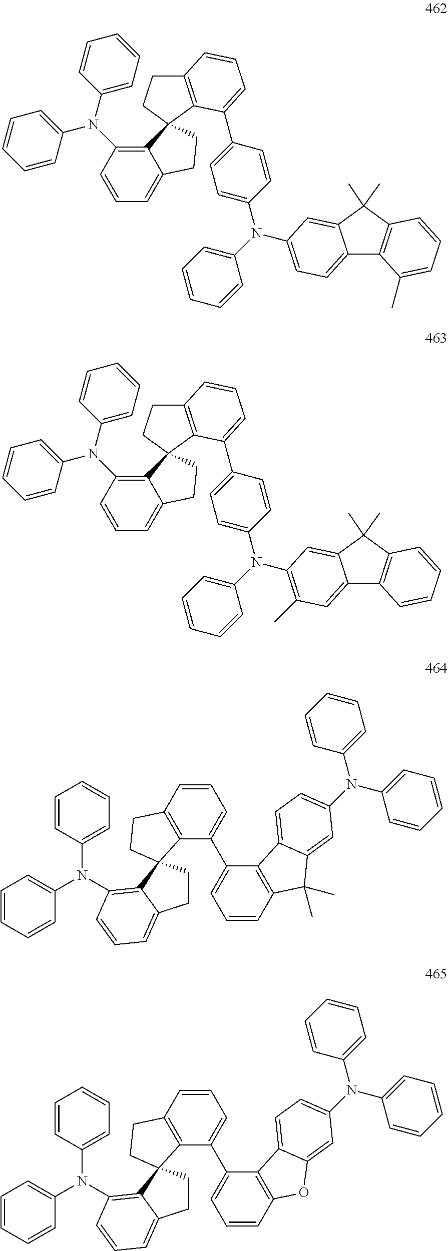 [SMILES: Cc1cccc2c1-c1ccc(N(c3ccccc3)c3ccc(-c4cccc5c4[C@@]4(CC5)CCc5cccc(N(c6ccccc6)c6ccccc6)c54)cc3)cc1C2(C)C ; Cc1cc2c(cc1N(c1ccccc1)c1ccc(-c3cccc4c3[C@@]3(CC4)CCc4cccc(N(c5ccccc5)c5ccccc5)c43)cc1)C(C)(C)c1ccccc1-2 ; CC1(C)c2cc(N(c3ccccc3)c3ccccc3)ccc2-c2c(-c3cccc4c3[C@@]3(CC4)CCc4cccc(N(c5ccccc5)c5ccccc5)c43)cccc21 ; c1ccc(N(c2ccccc2)c2ccc3c(c2)oc2cccc(-c4cccc5c4[C@@]4(CC5)CCc5cccc(N(c6ccccc6)c6ccccc6)c54)c23)cc1]

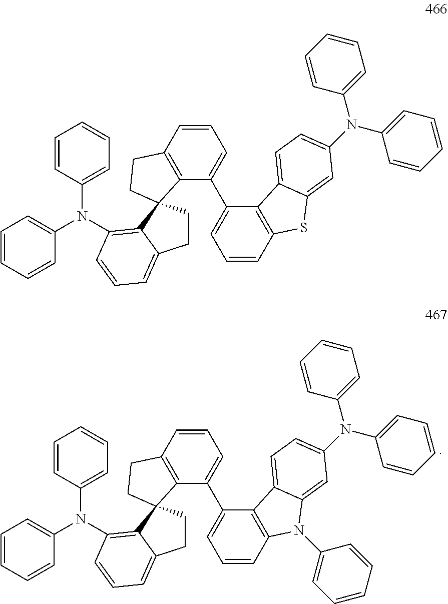 [SMILES: c1ccc(N(c2ccccc2)c2ccc3c(c2)sc2cccc(-c4cccc5c4[C@@]4(CC5)CCc5cccc(N(c6ccccc6)c6ccccc6)c54)c23)cc1 ; c1ccc(N(c2ccccc2)c2ccc3c4c(-c5cccc6c5[C@@]5(CC6)CCc6cccc(N(c7ccccc7)c7ccccc7)c65)cccc4n(-c4ccccc4)c3c2)cc1]

15. An amine compound represented by the following Formula 1: 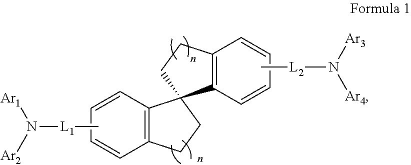
in Formula 1,
Ar₁ to Ar₄ are each independently a substituted or unsubstituted hydrocarbon ring of 6 to 30 ring-forming carbon atoms, or a substituted or unsubstituted heteroaryl group of 2 to 30 ring-forming atoms,
L₁ and L₂ are each independently a direct linkage, a substituted or unsubstituted arylene group of 6 to 30 ring-forming carbon atoms, or a substituted or unsubstituted heteroarylene group of 2 to 30 ring-forming carbon atoms, and
“n” is an integer of 1 to 3.

16. The amine compound of claim 15, wherein Formula 1 is represented by Formula 2-1 or Formula 2-2: 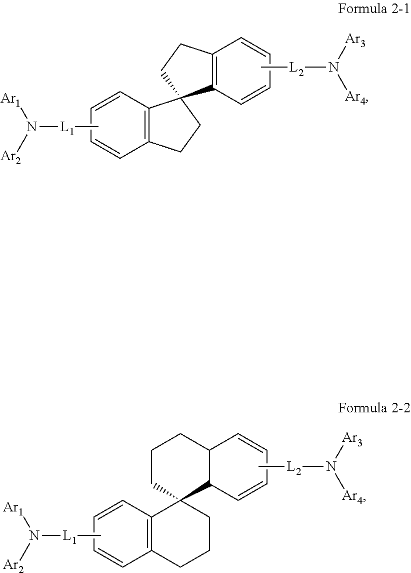
and
in Formula 2-1 and Formula 2-2,
Ar₁ to Ar₄, L₁ and L₂ are the same as defined in Formula 1.

17. The amine compound of claim 15, wherein Formula 1 is represented by Formula 3-1 or Formula 3-2: 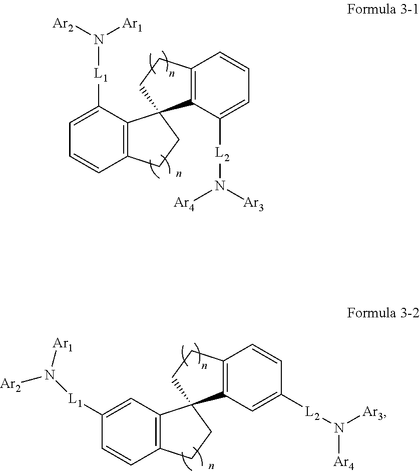
and
in Formula 3-1 and Formula 3-2,
Ar₁ to Ar₄, L₁, L₂, and “n” are the same as defined in Formula 1.

18. The amine compound of claim 15, wherein Formula 1 is represented by the following Formula 4-1: 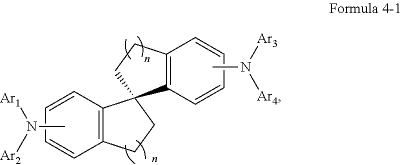
and
in Formula 4-1,
Ar₁ to Ar₄, and “n” are the same as defined in Formula 1.

19. The amine compound of claim 15, wherein Ar₁ to Ar₄ are each independently represented by Formula 1-1a or Formula 1-1b: 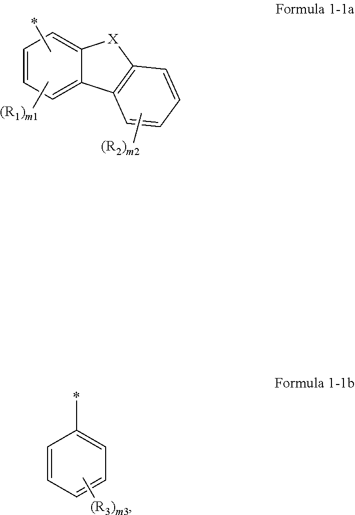
in Formula 1-1a,
X is O, S, CRₓR_{y}, or NR_{z}, and
Rₓ, R_{y}, and R_{z} are each independently a hydrogen atom, a deuterium atom, a substituted or unsubstituted alkyl group of 1 to 10 carbon atoms, or a substituted or unsubstituted aryl group of 6 to 15 ring-forming carbon atoms, and
in Formula 1-1a and Formula 1-1b,
R₁ to R₃ are each independently a hydrogen atom, a deuterium atom, a substituted or unsubstituted alkyl group of 1 to 10 carbon atoms, a substituted or unsubstituted cycloalkyl group of 6 to 15 ring-forming carbon atoms, or a substituted or unsubstituted aryl group of 6 to 15 ring-forming carbon atoms, and/or combined with an adjacent group to form a ring,
m1 is an integer of 0 to 3, m2 is an integer of 0 to 4, and
m3 is an integer of 0 to 5.

20. The amine compound of claim 19, wherein Formula 1 is represented by Formula 5-1 or Formula 5-2: 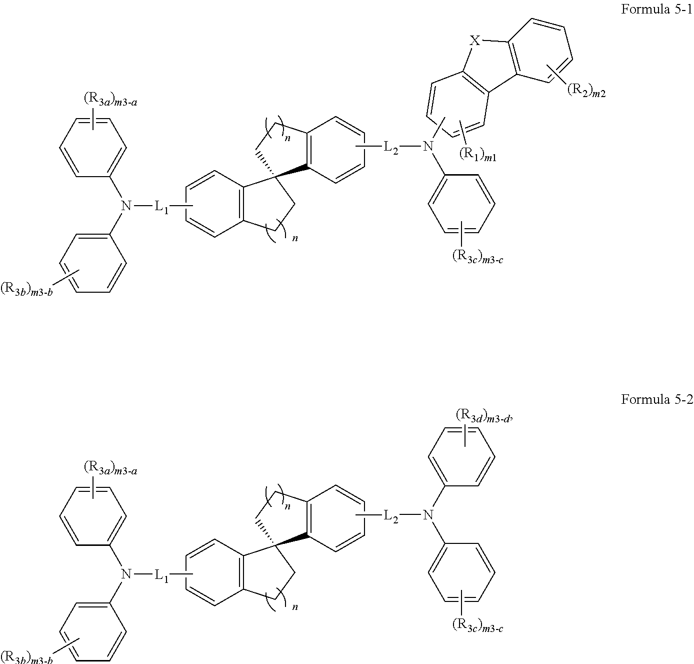
in Formula 5-1 and Formula 5-2,
L₁, L₂, and “n” are the same as defined in Formula 1,
X, R₁, R₂, m1 and m2 are the same as defined in Formula 1-1a,
R₃ₐ, R_{3b}, R_{3c}, and R_{3d} are each independently defined the same as R₃ is defined in Formula 1-1b, and
m3-a, m3-b, m3-c, and m3-d are each independently defined the same as m3 is defined in Formula 1-1b.

21. The amine compound of claim 20, wherein Formula 5-1 is represented by Formula 5-1a or Formula 5-1b: 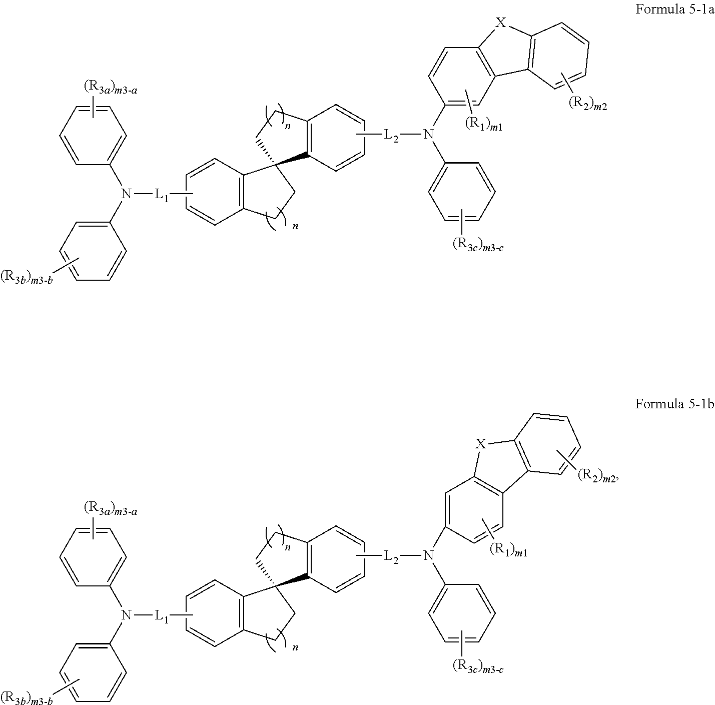
and
in Formula 5-1a and Formula 5-1b,
L₁, L₂, X, R₁, R₂, R₃ₐ, R_{3b}, R_{3c}, n, m1, m2, m3-a, m3-b, and m3-c are the same as defined in Formula 5-1.

22. The amine compound of claim 15, wherein Formula 1 is represented by any one selected from compounds in Compound Group 1:

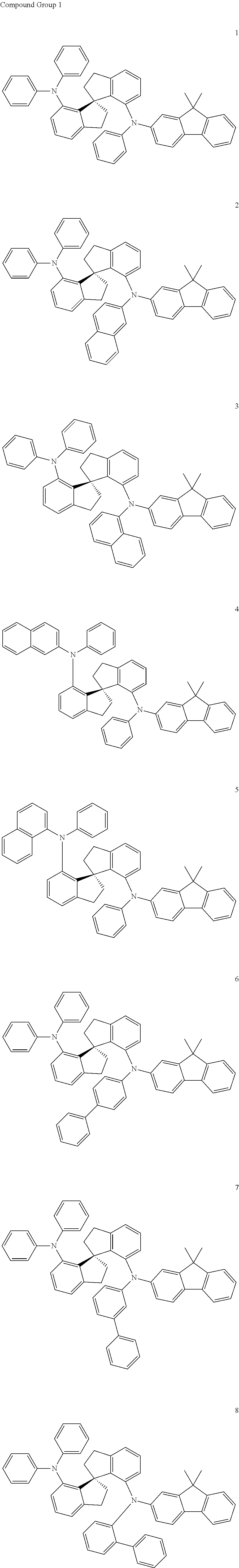

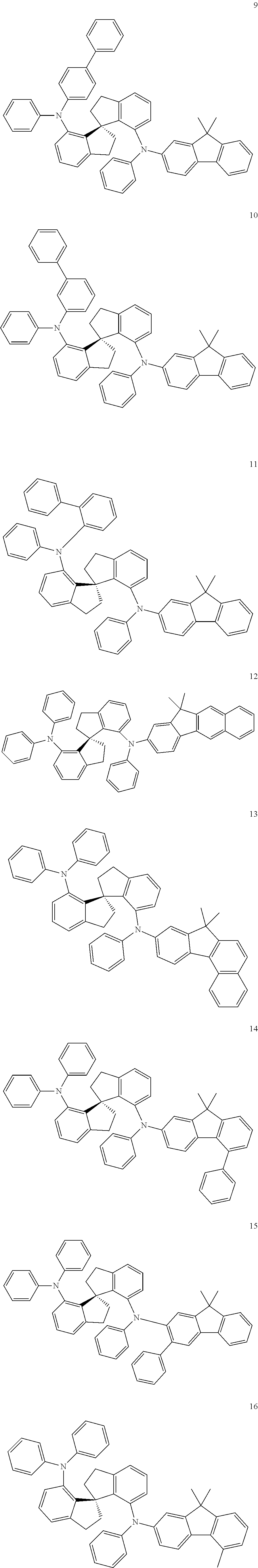

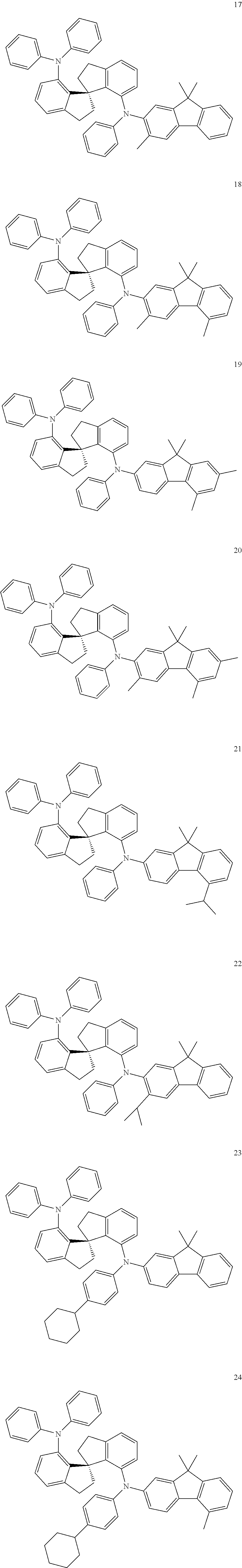

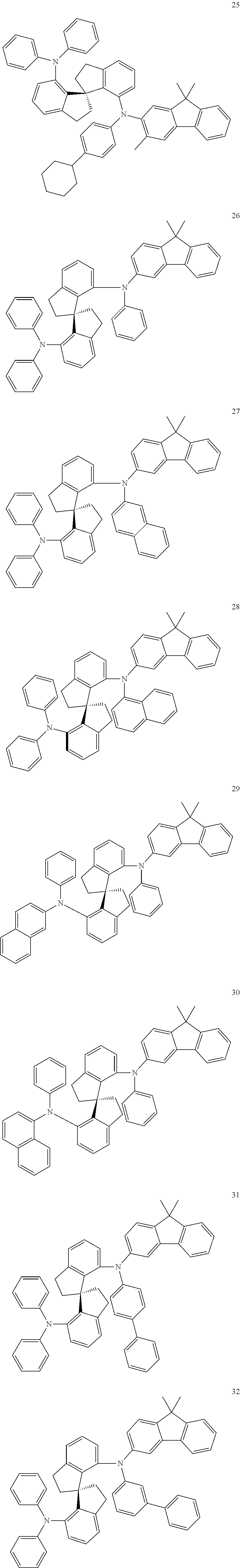

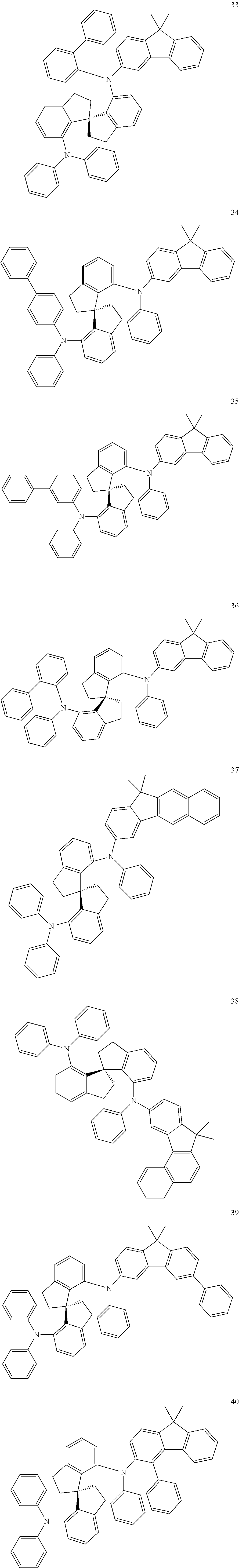

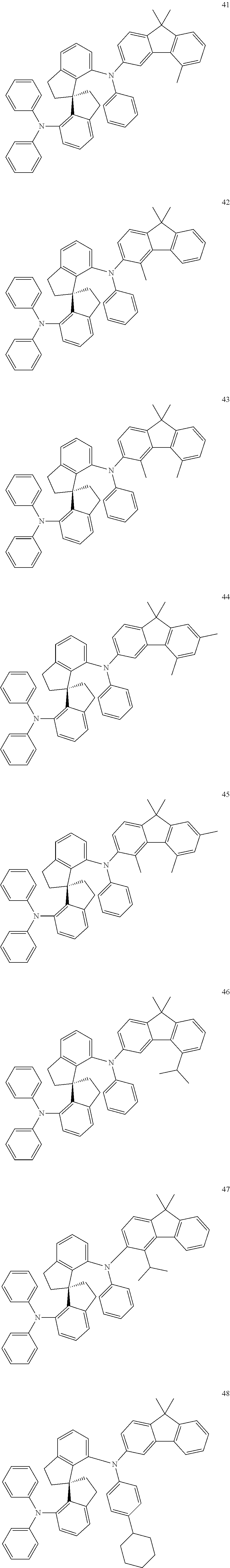

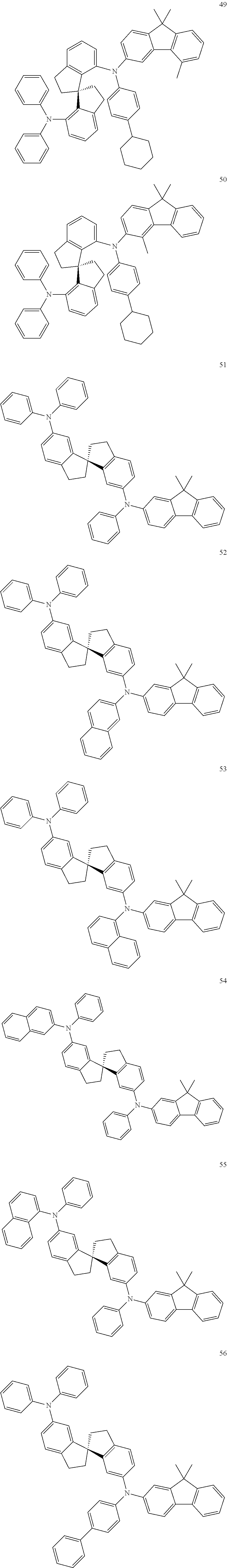

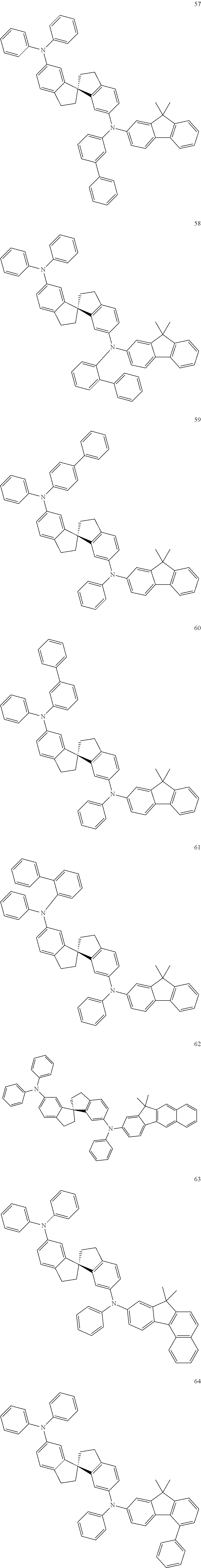

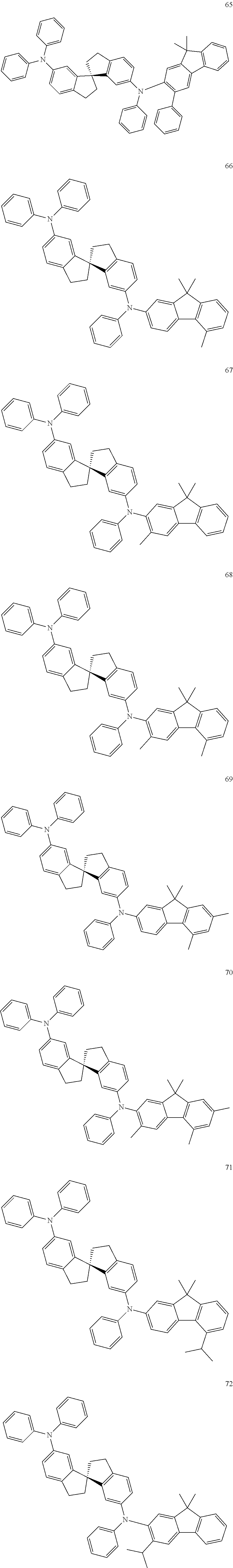

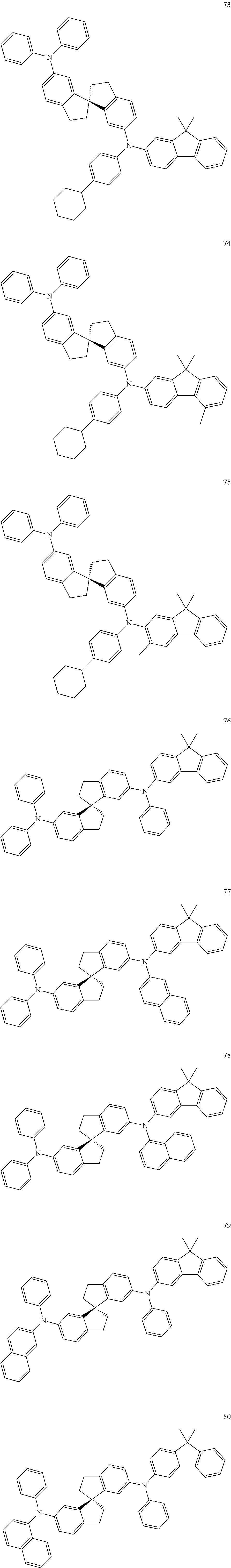

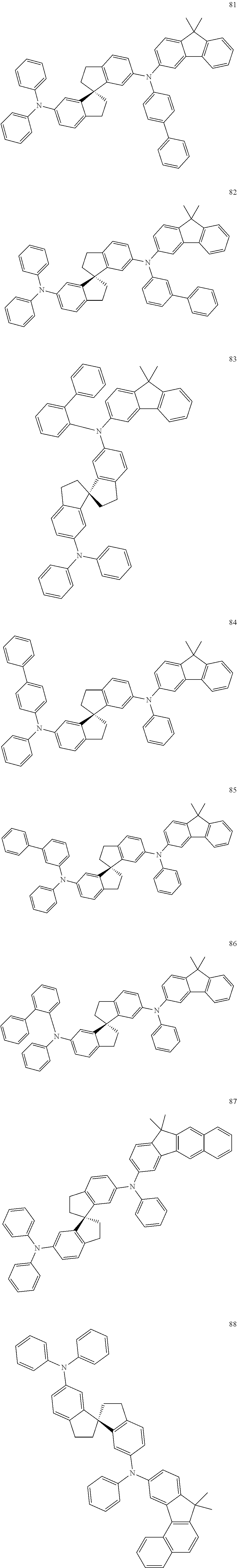

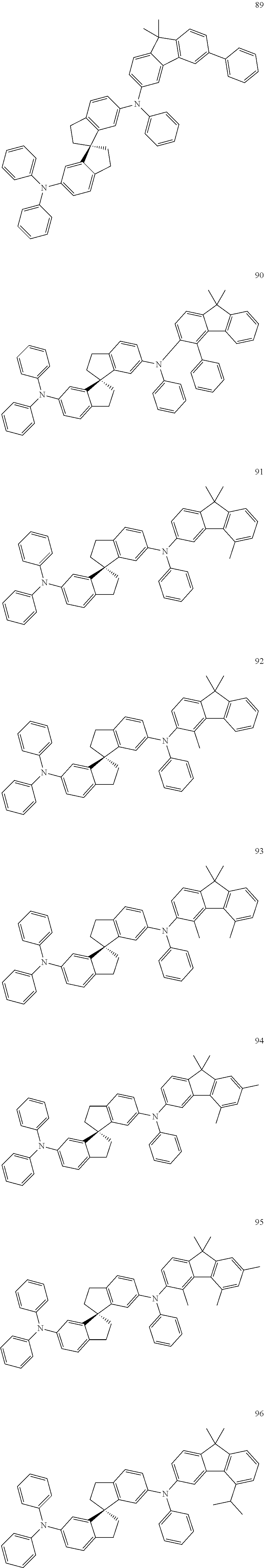

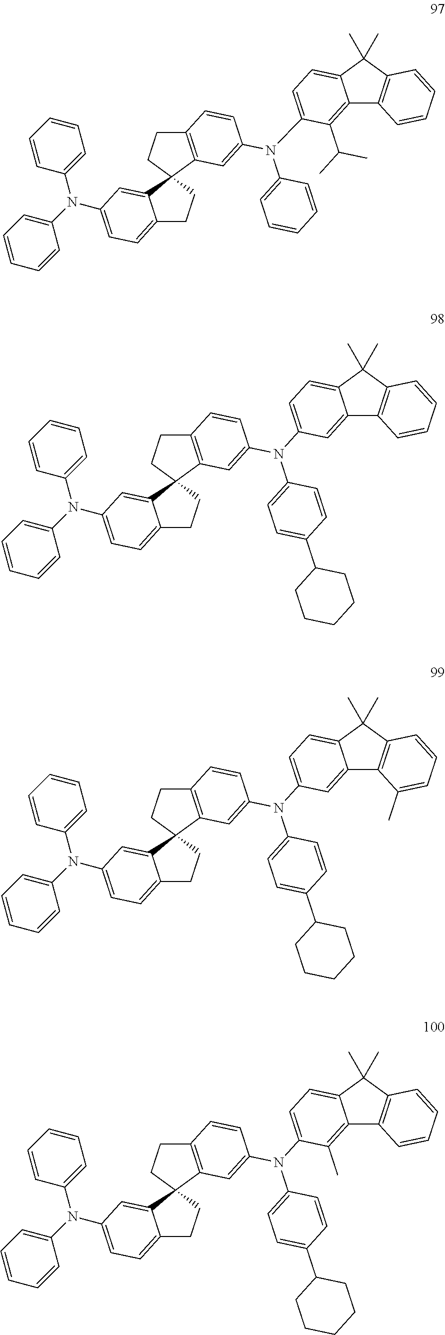 [SMILES: CC(C)c1c(N(c2ccccc2)c2ccc3c(c2)[C@]2(CCc4ccc(N(c5ccccc5)c5ccccc5)cc42)CC3)ccc2c1-c1ccccc1C2(C)C ; CC1(C)c2ccccc2-c2cc(N(c3ccc(C4CCCCC4)cc3)c3ccc4c(c3)[C@]3(CCc5ccc(N(c6ccccc6)c6ccccc6)cc53)CC4)ccc21 ; Cc1cccc2c1-c1cc(N(c3ccc(C4CCCCC4)cc3)c3ccc4c(c3)[C@]3(CCc5ccc(N(c6ccccc6)c6ccccc6)cc53)CC4)ccc1C2(C)C ; Cc1c(N(c2ccc(C3CCCCC3)cc2)c2ccc3c(c2)[C@]2(CCc4ccc(N(c5ccccc5)c5ccccc5)cc42)CC3)ccc2c1-c1ccccc1C2(C)C]

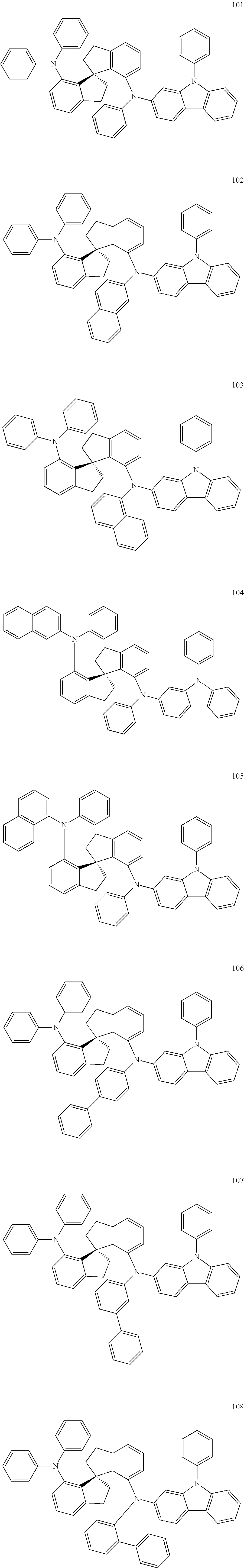

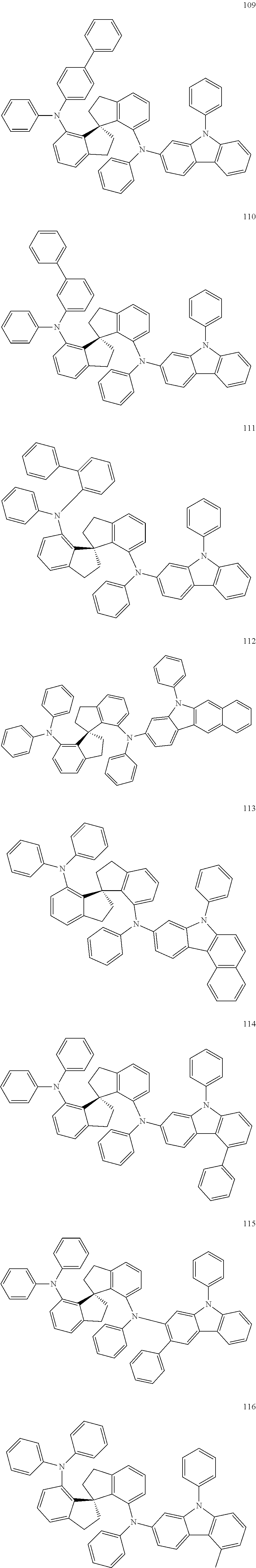

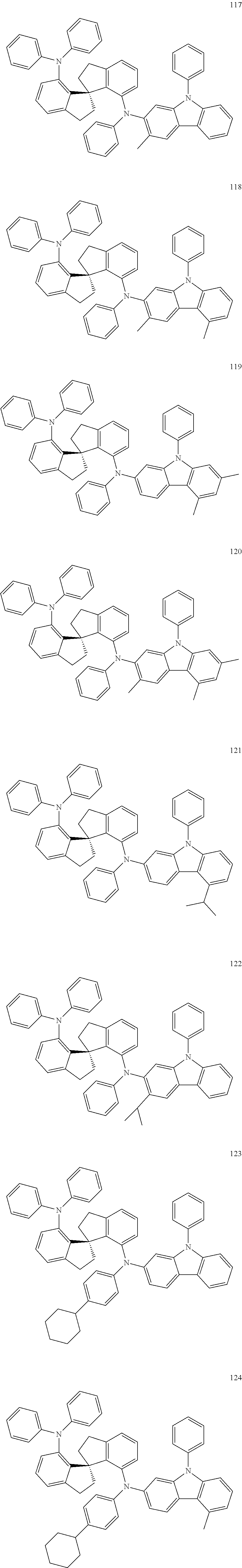

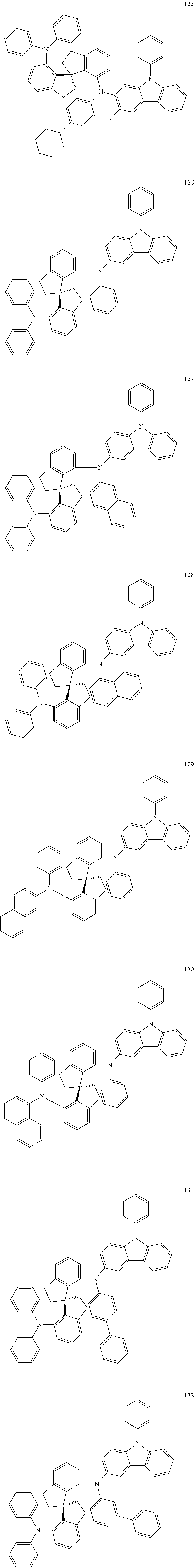

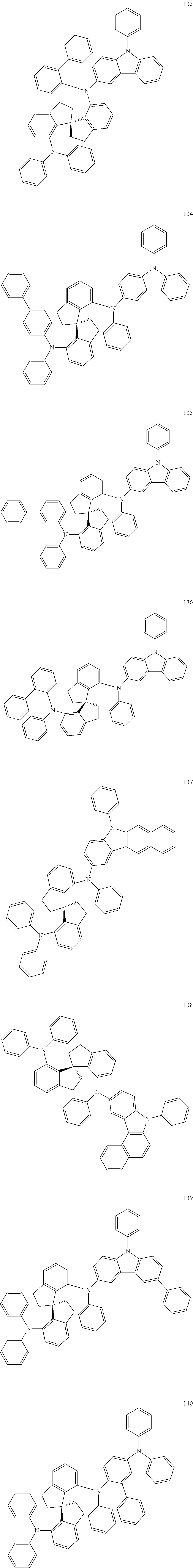

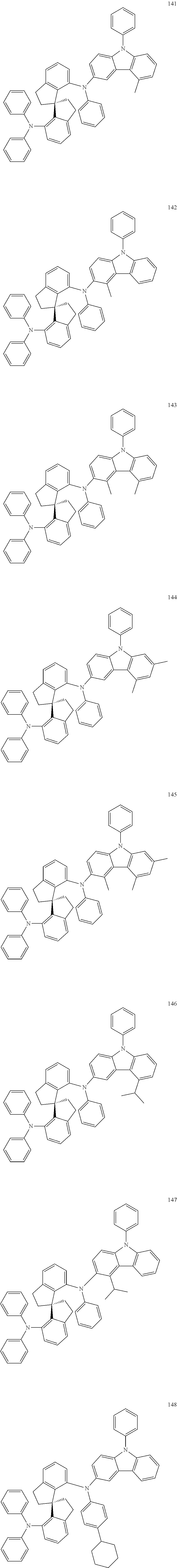

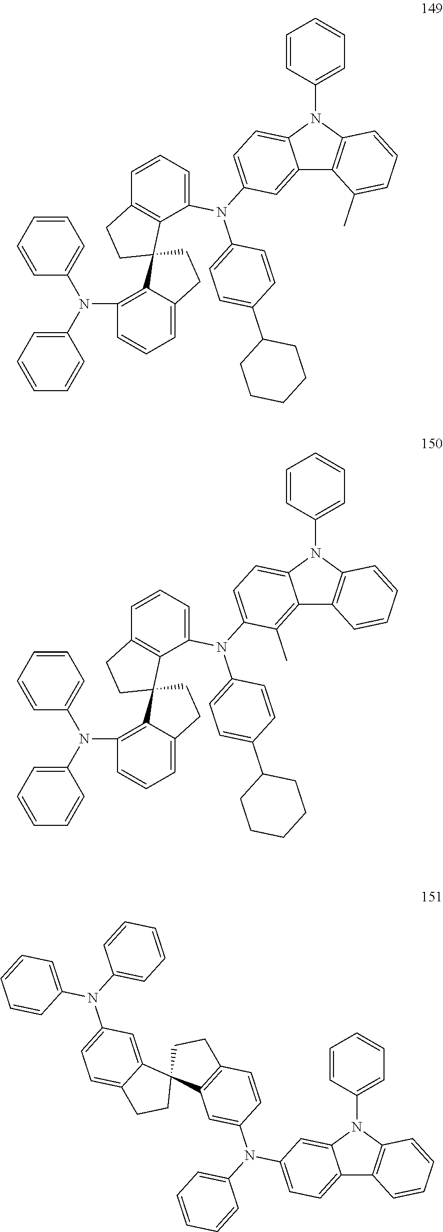 [SMILES: Cc1cccc2c1c1cc(N(c3ccc(C4CCCCC4)cc3)c3cccc4c3[C@]3(CCc5cccc(N(c6ccccc6)c6ccccc6)c53)CC4)ccc1n2-c1ccccc1 ; Cc1c(N(c2ccc(C3CCCCC3)cc2)c2cccc3c2[C@]2(CCc4cccc(N(c5ccccc5)c5ccccc5)c42)CC3)ccc2c1c1ccccc1n2-c1ccccc1 ; c1ccc(N(c2ccccc2)c2ccc3c(c2)[C@@]2(CC3)CCc3ccc(N(c4ccccc4)c4ccc5c6ccccc6n(-c6ccccc6)c5c4)cc32)cc1]

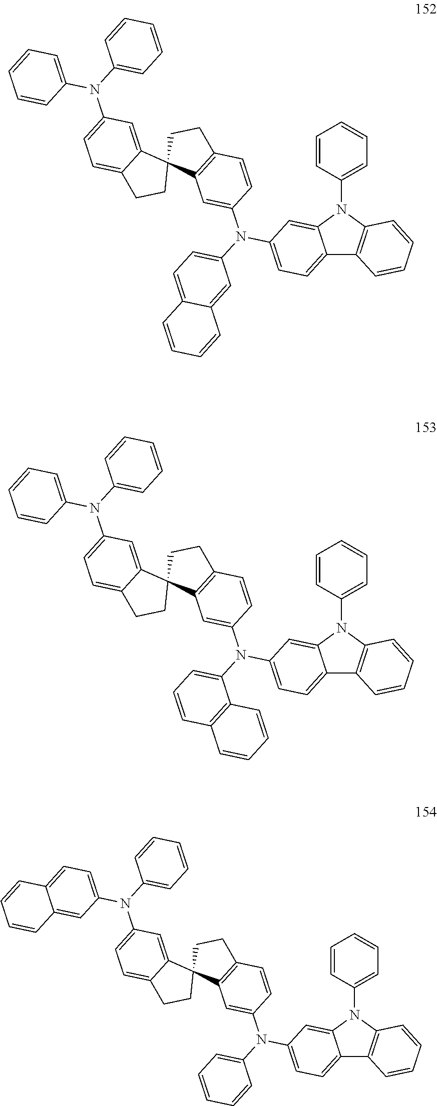 [SMILES: c1ccc(N(c2ccccc2)c2ccc3c(c2)[C@@]2(CC3)CCc3ccc(N(c4ccc5ccccc5c4)c4ccc5c6ccccc6n(-c6ccccc6)c5c4)cc32)cc1 ; c1ccc(N(c2ccccc2)c2ccc3c(c2)[C@@]2(CC3)CCc3ccc(N(c4ccc5c6ccccc6n(-c6ccccc6)c5c4)c4cccc5ccccc45)cc32)cc1 ; c1ccc(N(c2ccc3c(c2)[C@@]2(CC3)CCc3ccc(N(c4ccccc4)c4ccc5c6ccccc6n(-c6ccccc6)c5c4)cc32)c2ccc3ccccc3c2)cc1]

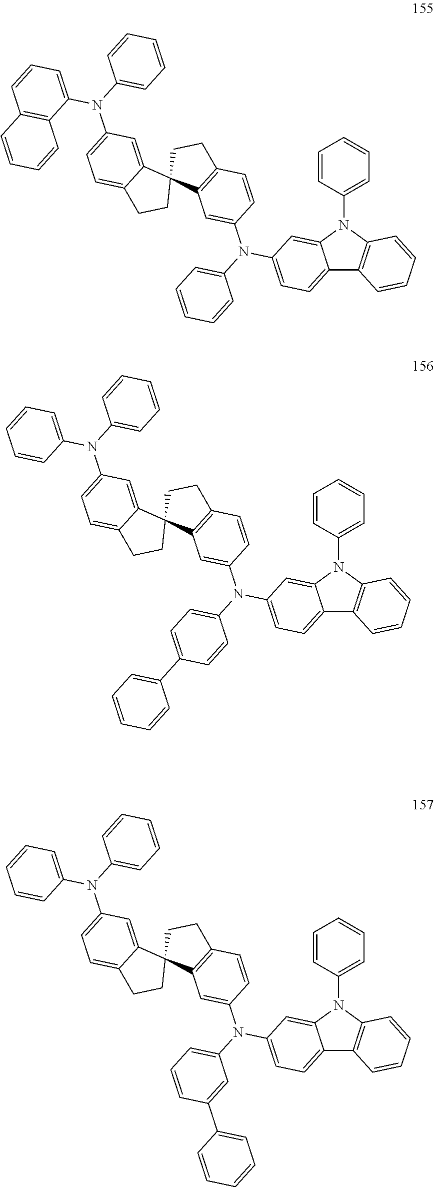 [SMILES: c1ccc(N(c2ccc3c(c2)[C@@]2(CC3)CCc3ccc(N(c4ccccc4)c4cccc5ccccc45)cc32)c2ccc3c4ccccc4n(-c4ccccc4)c3c2)cc1 ; c1ccc(-c2ccc(N(c3ccc4c(c3)[C@]3(CCc5ccc(N(c6ccccc6)c6ccccc6)cc53)CC4)c3ccc4c5ccccc5n(-c5ccccc5)c4c3)cc2)cc1 ; c1ccc(-c2cccc(N(c3ccc4c(c3)[C@]3(CCc5ccc(N(c6ccccc6)c6ccccc6)cc53)CC4)c3ccc4c5ccccc5n(-c5ccccc5)c4c3)c2)cc1]

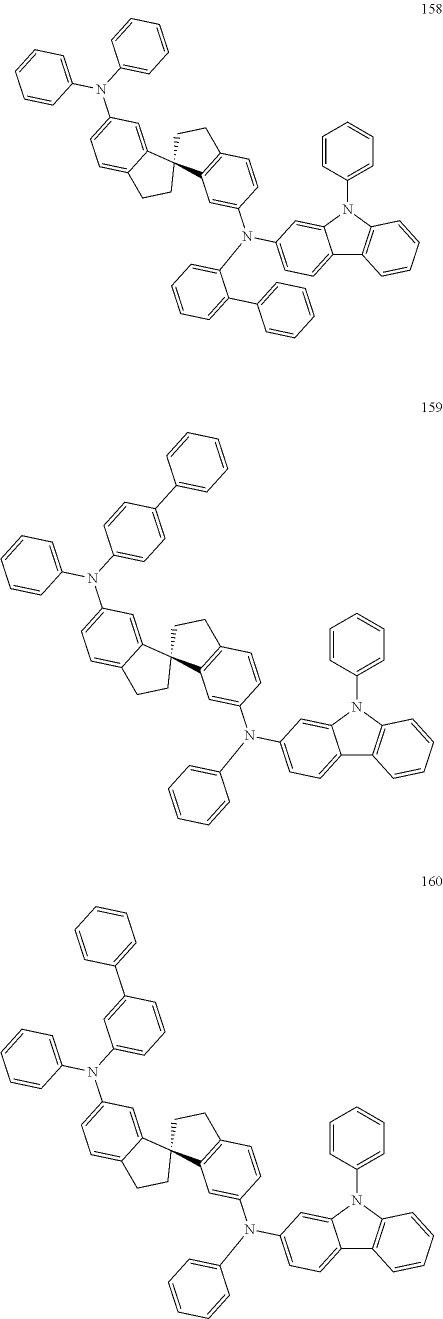 [SMILES: c1ccc(-c2ccccc2N(c2ccc3c(c2)[C@]2(CCc4ccc(N(c5ccccc5)c5ccccc5)cc42)CC3)c2ccc3c4ccccc4n(-c4ccccc4)c3c2)cc1 ; c1ccc(-c2ccc(N(c3ccccc3)c3ccc4c(c3)[C@@]3(CC4)CCc4ccc(N(c5ccccc5)c5ccc6c7ccccc7n(-c7ccccc7)c6c5)cc43)cc2)cc1 ; c1ccc(-c2cccc(N(c3ccccc3)c3ccc4c(c3)[C@@]3(CC4)CCc4ccc(N(c5ccccc5)c5ccc6c7ccccc7n(-c7ccccc7)c6c5)cc43)c2)cc1]

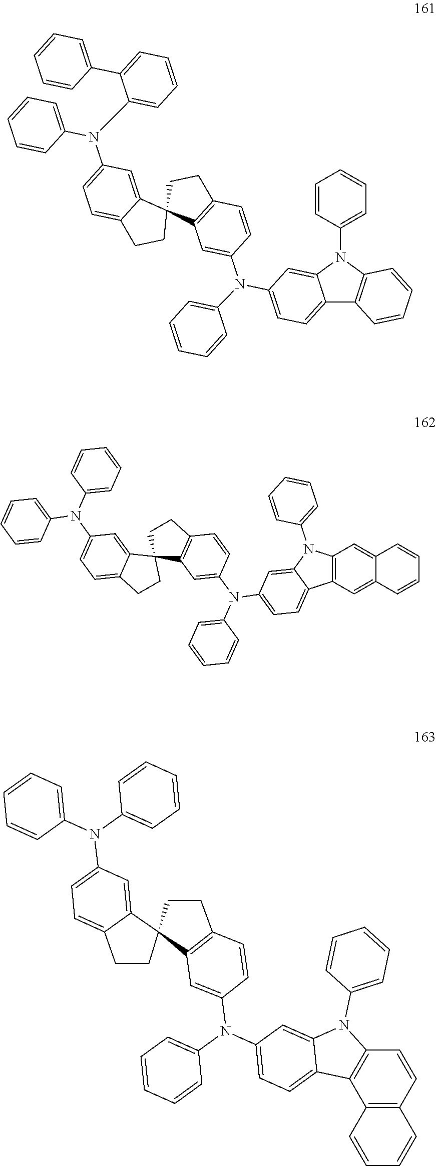 [SMILES: c1ccc(-c2ccccc2N(c2ccccc2)c2ccc3c(c2)[C@]2(CCc4ccc(N(c5ccccc5)c5ccc6c7ccccc7n(-c7ccccc7)c6c5)cc42)CC3)cc1 ; c1ccc(N(c2ccccc2)c2ccc3c(c2)[C@@]2(CC3)CCc3ccc(N(c4ccccc4)c4ccc5c6cc7ccccc7cc6n(-c6ccccc6)c5c4)cc32)cc1 ; c1ccc(N(c2ccccc2)c2ccc3c(c2)[C@@]2(CC3)CCc3ccc(N(c4ccccc4)c4ccc5c6c7ccccc7ccc6n(-c6ccccc6)c5c4)cc32)cc1]

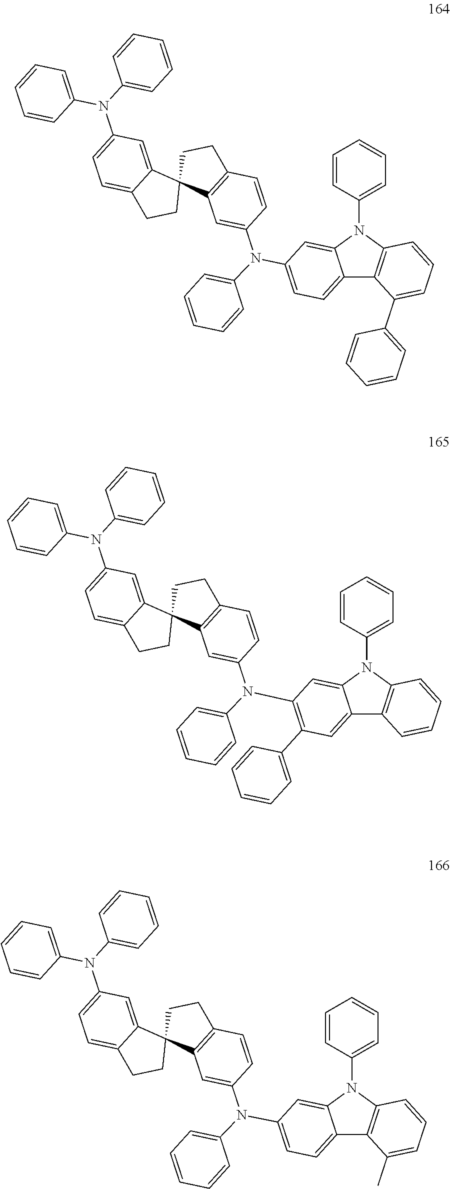 [SMILES: c1ccc(-c2cccc3c2c2ccc(N(c4ccccc4)c4ccc5c(c4)[C@]4(CCc6ccc(N(c7ccccc7)c7ccccc7)cc64)CC5)cc2n3-c2ccccc2)cc1 ; c1ccc(-c2cc3c4ccccc4n(-c4ccccc4)c3cc2N(c2ccccc2)c2ccc3c(c2)[C@]2(CCc4ccc(N(c5ccccc5)c5ccccc5)cc42)CC3)cc1 ; Cc1cccc2c1c1ccc(N(c3ccccc3)c3ccc4c(c3)[C@]3(CCc5ccc(N(c6ccccc6)c6ccccc6)cc53)CC4)cc1n2-c1ccccc1]

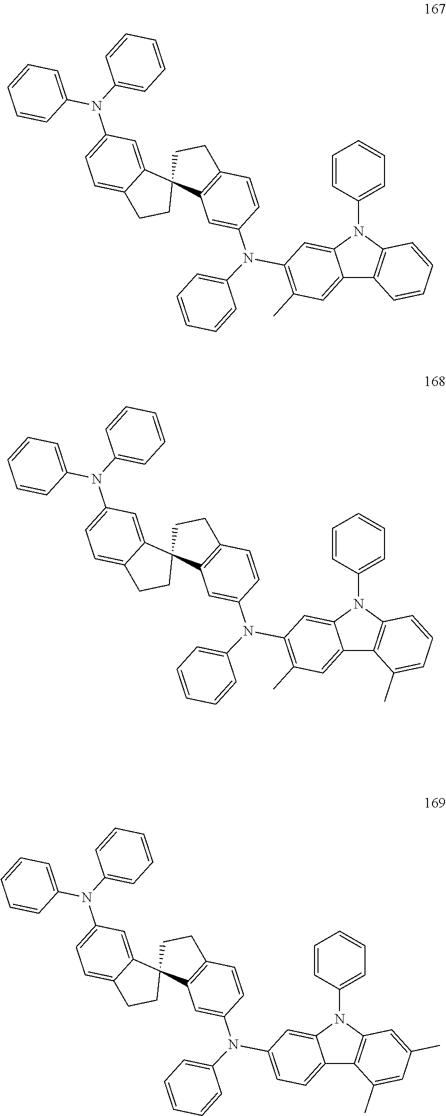 [SMILES: Cc1cc2c3ccccc3n(-c3ccccc3)c2cc1N(c1ccccc1)c1ccc2c(c1)[C@]1(CCc3ccc(N(c4ccccc4)c4ccccc4)cc31)CC2 ; Cc1cc2c3c(C)cccc3n(-c3ccccc3)c2cc1N(c1ccccc1)c1ccc2c(c1)[C@]1(CCc3ccc(N(c4ccccc4)c4ccccc4)cc31)CC2 ; Cc1cc(C)c2c3ccc(N(c4ccccc4)c4ccc5c(c4)[C@]4(CCc6ccc(N(c7ccccc7)c7ccccc7)cc64)CC5)cc3n(-c3ccccc3)c2c1]

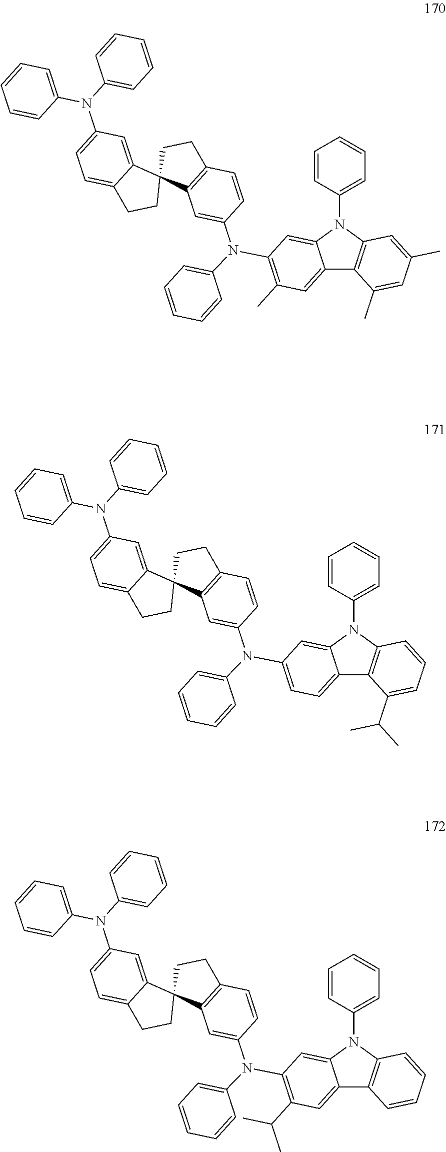 [SMILES: Cc1cc(C)c2c3cc(C)c(N(c4ccccc4)c4ccc5c(c4)[C@]4(CCc6ccc(N(c7ccccc7)c7ccccc7)cc64)CC5)cc3n(-c3ccccc3)c2c1 ; CC(C)c1cccc2c1c1ccc(N(c3ccccc3)c3ccc4c(c3)[C@]3(CCc5ccc(N(c6ccccc6)c6ccccc6)cc53)CC4)cc1n2-c1ccccc1 ; CC(C)c1cc2c3ccccc3n(-c3ccccc3)c2cc1N(c1ccccc1)c1ccc2c(c1)[C@]1(CCc3ccc(N(c4ccccc4)c4ccccc4)cc31)CC2]

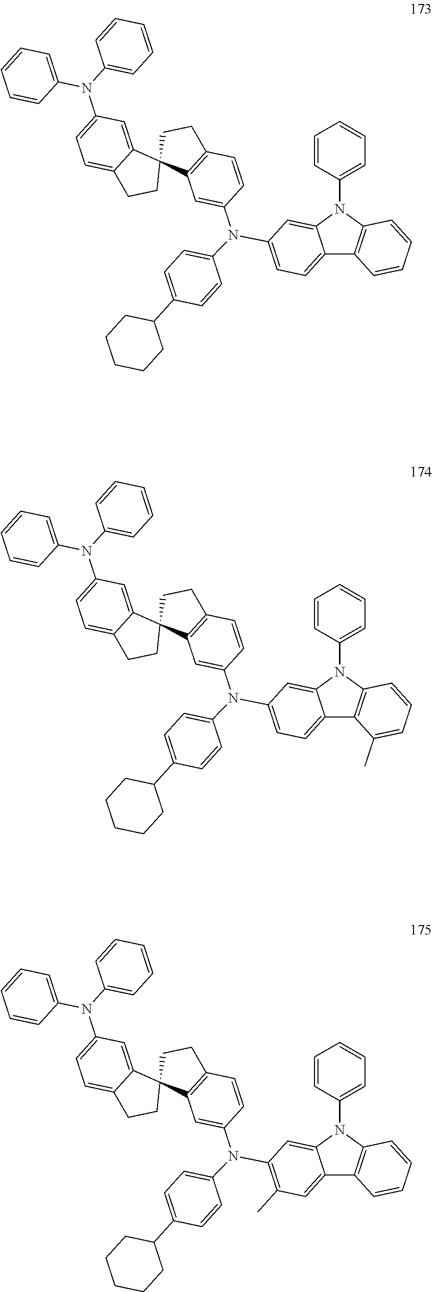 [SMILES: c1ccc(N(c2ccccc2)c2ccc3c(c2)[C@@]2(CC3)CCc3ccc(N(c4ccc(C5CCCCC5)cc4)c4ccc5c6ccccc6n(-c6ccccc6)c5c4)cc32)cc1 ; Cc1cccc2c1c1ccc(N(c3ccc(C4CCCCC4)cc3)c3ccc4c(c3)[C@]3(CCc5ccc(N(c6ccccc6)c6ccccc6)cc53)CC4)cc1n2-c1ccccc1 ; Cc1cc2c3ccccc3n(-c3ccccc3)c2cc1N(c1ccc(C2CCCCC2)cc1)c1ccc2c(c1)[C@]1(CCc3ccc(N(c4ccccc4)c4ccccc4)cc31)CC2]

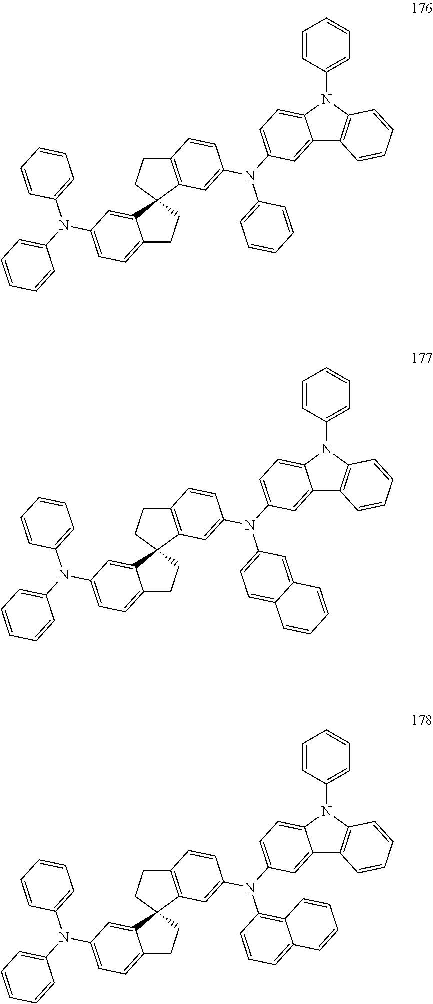 [SMILES: c1ccc(N(c2ccccc2)c2ccc3c(c2)[C@@]2(CC3)CCc3ccc(N(c4ccccc4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5ccccc5c4)c4ccc5c(c4)c4ccccc4n5-c4ccccc4)cc32)cc1 ; c1ccc(N(c2ccccc2)c2ccc3c(c2)[C@@]2(CC3)CCc3ccc(N(c4ccc5c(c4)c4ccccc4n5-c4ccccc4)c4cccc5ccccc45)cc32)cc1]

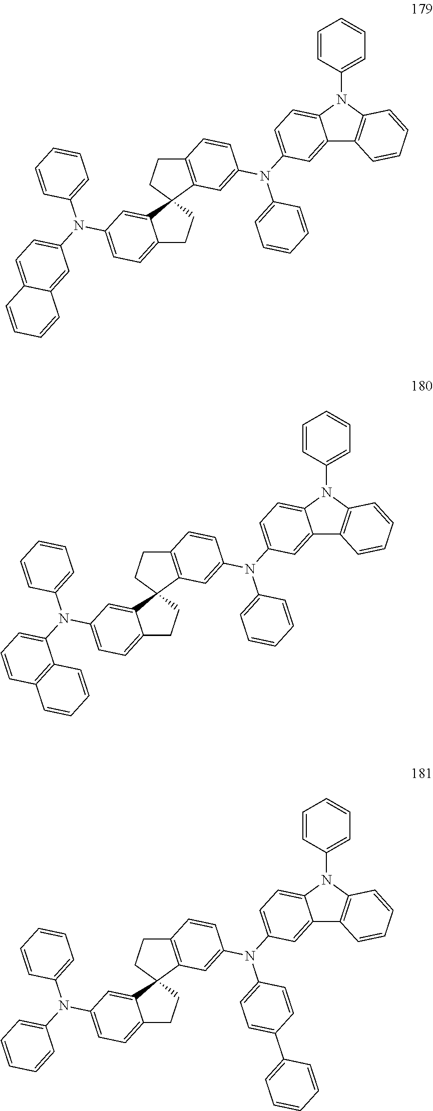 [SMILES: c1ccc(N(c2ccc3c(c2)[C@@]2(CC3)CCc3ccc(N(c4ccccc4)c4ccc5c(c4)c4ccccc4n5-c4ccccc4)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)c2ccccc2n3-c2ccccc2)cc1 ; c1ccc(-c2ccc(N(c3ccc4c(c3)[C@]3(CCc5ccc(N(c6ccccc6)c6ccccc6)cc53)CC4)c3ccc4c(c3)c3ccccc3n4-c3ccccc3)cc2)cc1]

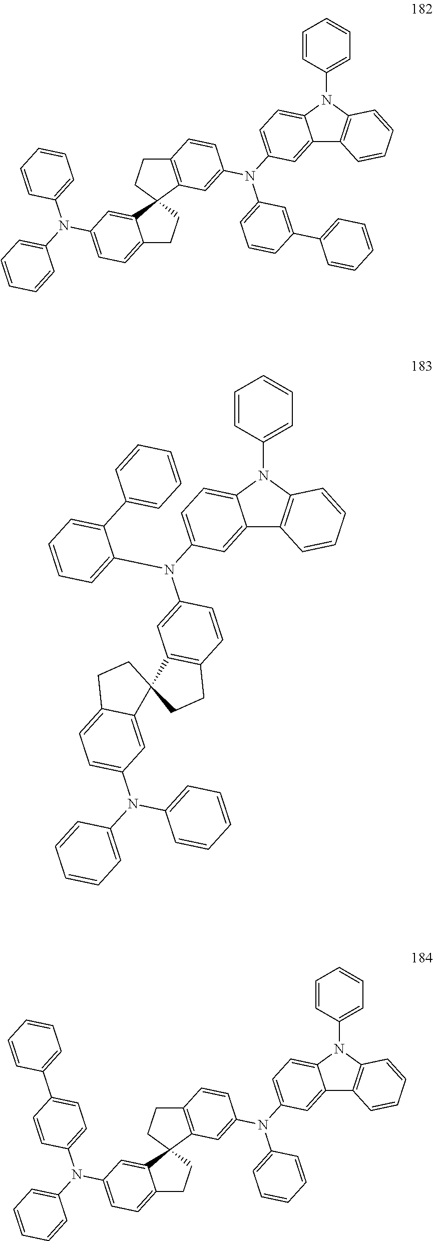 [SMILES: c1ccc(-c2cccc(N(c3ccc4c(c3)[C@]3(CCc5ccc(N(c6ccccc6)c6ccccc6)cc53)CC4)c3ccc4c(c3)c3ccccc3n4-c3ccccc3)c2)cc1 ; c1ccc(-c2ccccc2N(c2ccc3c(c2)[C@]2(CCc4ccc(N(c5ccccc5)c5ccccc5)cc42)CC3)c2ccc3c(c2)c2ccccc2n3-c2ccccc2)cc1 ; c1ccc(-c2ccc(N(c3ccccc3)c3ccc4c(c3)[C@@]3(CC4)CCc4ccc(N(c5ccccc5)c5ccc6c(c5)c5ccccc5n6-c5ccccc5)cc43)cc2)cc1]

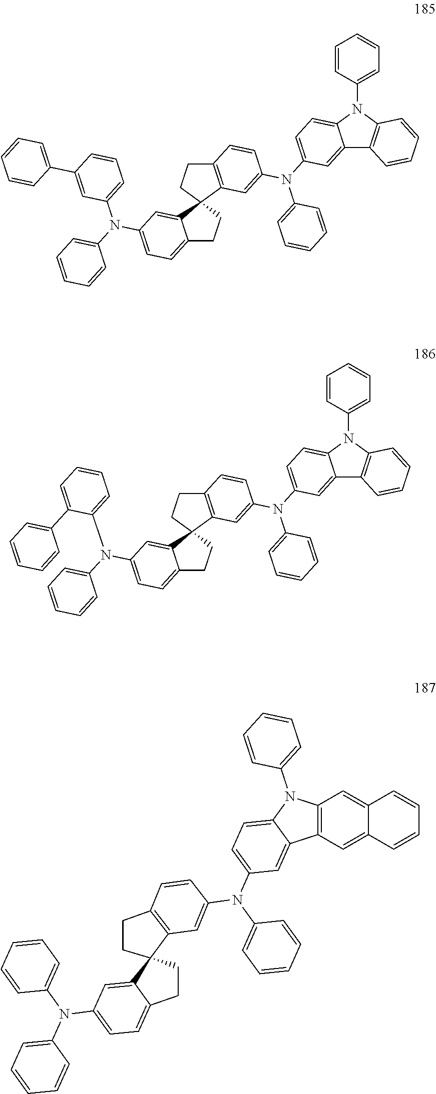 [SMILES: c1ccc(-c2cccc(N(c3ccccc3)c3ccc4c(c3)[C@@]3(CC4)CCc4ccc(N(c5ccccc5)c5ccc6c(c5)c5ccccc5n6-c5ccccc5)cc43)c2)cc1 ; c1ccc(-c2ccccc2N(c2ccccc2)c2ccc3c(c2)[C@]2(CCc4ccc(N(c5ccccc5)c5ccc6c(c5)c5ccccc5n6-c5ccccc5)cc42)CC3)cc1 ; c1ccc(N(c2ccccc2)c2ccc3c(c2)[C@@]2(CC3)CCc3ccc(N(c4ccccc4)c4ccc5c(c4)c4cc6ccccc6cc4n5-c4ccccc4)cc32)cc1]

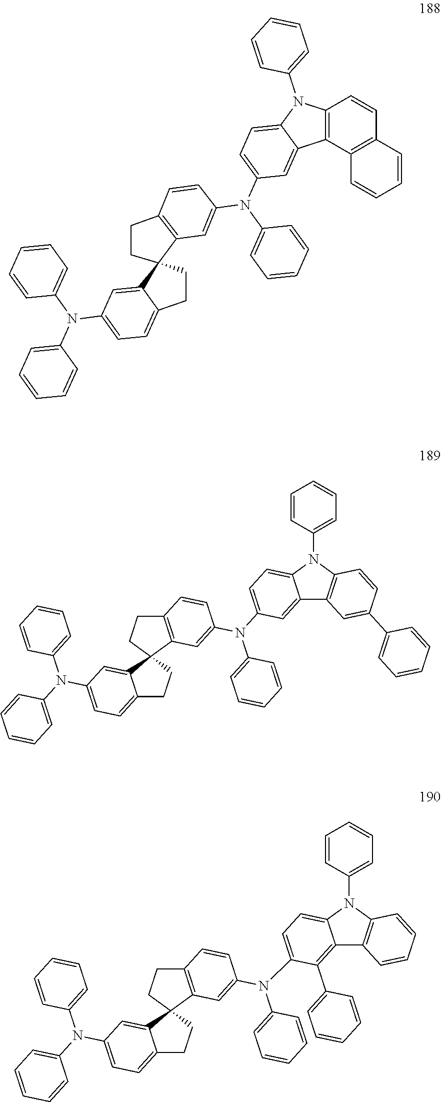 [SMILES: c1ccc(N(c2ccccc2)c2ccc3c(c2)[C@@]2(CC3)CCc3ccc(N(c4ccccc4)c4ccc5c(c4)c4c6ccccc6ccc4n5-c4ccccc4)cc32)cc1 ; c1ccc(-c2ccc3c(c2)c2cc(N(c4ccccc4)c4ccc5c(c4)[C@]4(CCc6ccc(N(c7ccccc7)c7ccccc7)cc64)CC5)ccc2n3-c2ccccc2)cc1 ; c1ccc(-c2c(N(c3ccccc3)c3ccc4c(c3)[C@]3(CCc5ccc(N(c6ccccc6)c6ccccc6)cc53)CC4)ccc3c2c2ccccc2n3-c2ccccc2)cc1]

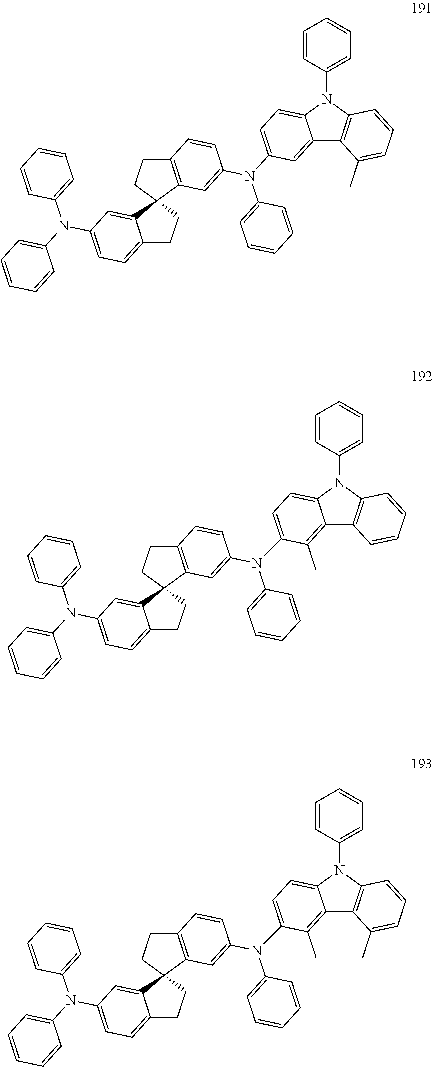 [SMILES: Cc1cccc2c1c1cc(N(c3ccccc3)c3ccc4c(c3)[C@]3(CCc5ccc(N(c6ccccc6)c6ccccc6)cc53)CC4)ccc1n2-c1ccccc1 ; Cc1c(N(c2ccccc2)c2ccc3c(c2)[C@]2(CCc4ccc(N(c5ccccc5)c5ccccc5)cc42)CC3)ccc2c1c1ccccc1n2-c1ccccc1 ; Cc1cccc2c1c1c(C)c(N(c3ccccc3)c3ccc4c(c3)[C@]3(CCc5ccc(N(c6ccccc6)c6ccccc6)cc53)CC4)ccc1n2-c1ccccc1]

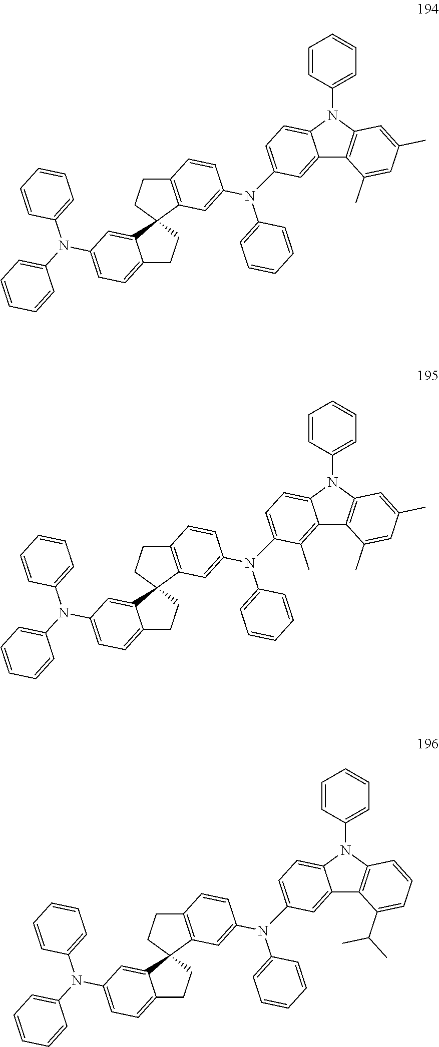 [SMILES: Cc1cc(C)c2c3cc(N(c4ccccc4)c4ccc5c(c4)[C@]4(CCc6ccc(N(c7ccccc7)c7ccccc7)cc64)CC5)ccc3n(-c3ccccc3)c2c1 ; Cc1cc(C)c2c3c(C)c(N(c4ccccc4)c4ccc5c(c4)[C@]4(CCc6ccc(N(c7ccccc7)c7ccccc7)cc64)CC5)ccc3n(-c3ccccc3)c2c1 ; CC(C)c1cccc2c1c1cc(N(c3ccccc3)c3ccc4c(c3)[C@]3(CCc5ccc(N(c6ccccc6)c6ccccc6)cc53)CC4)ccc1n2-c1ccccc1]

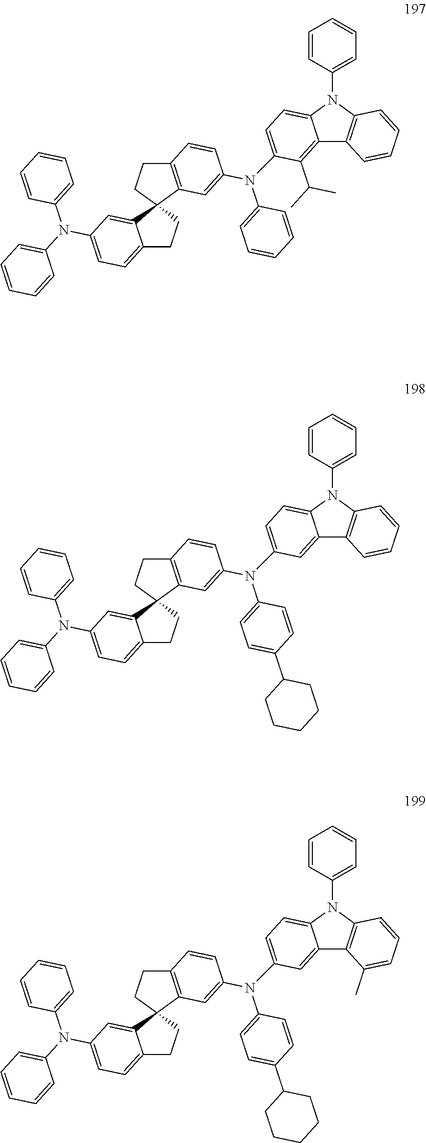 [SMILES: CC(C)c1c(N(c2ccccc2)c2ccc3c(c2)[C@]2(CCc4ccc(N(c5ccccc5)c5ccccc5)cc42)CC3)ccc2c1c1ccccc1n2-c1ccccc1 ; c1ccc(N(c2ccccc2)c2ccc3c(c2)[C@@]2(CC3)CCc3ccc(N(c4ccc(C5CCCCC5)cc4)c4ccc5c(c4)c4ccccc4n5-c4ccccc4)cc32)cc1 ; Cc1cccc2c1c1cc(N(c3ccc(C4CCCCC4)cc3)c3ccc4c(c3)[C@]3(CCc5ccc(N(c6ccccc6)c6ccccc6)cc53)CC4)ccc1n2-c1ccccc1]

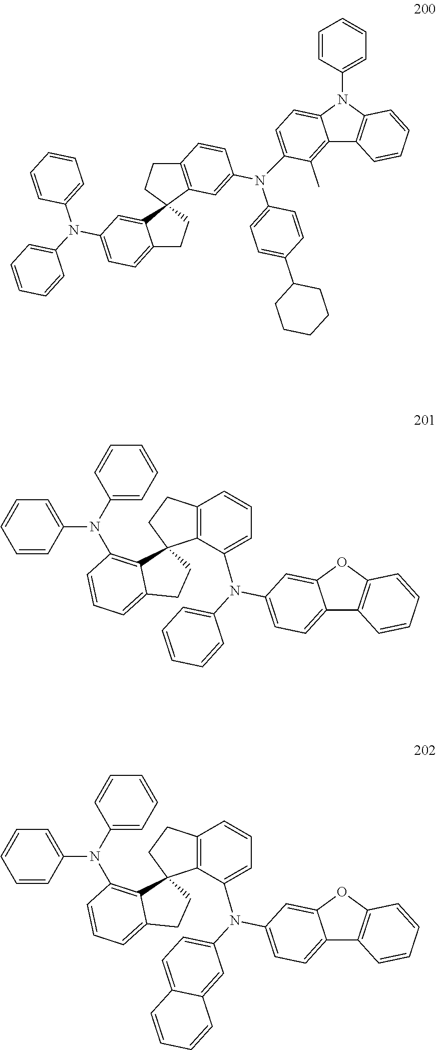 [SMILES: Cc1c(N(c2ccc(C3CCCCC3)cc2)c2ccc3c(c2)[C@]2(CCc4ccc(N(c5ccccc5)c5ccccc5)cc42)CC3)ccc2c1c1ccccc1n2-c1ccccc1 ; c1ccc(N(c2ccccc2)c2cccc3c2[C@@]2(CC3)CCc3cccc(N(c4ccccc4)c4ccc5c(c4)oc4ccccc45)c32)cc1 ; c1ccc(N(c2ccccc2)c2cccc3c2[C@@]2(CC3)CCc3cccc(N(c4ccc5ccccc5c4)c4ccc5c(c4)oc4ccccc45)c32)cc1]

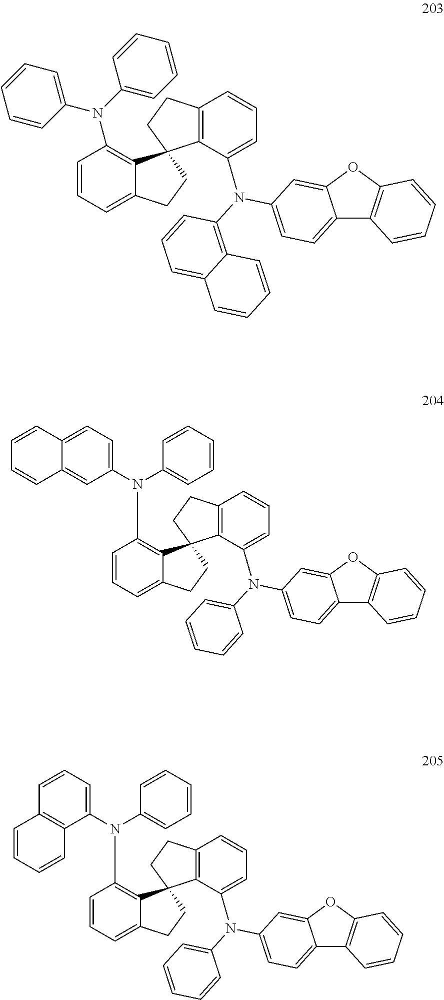 [SMILES: c1ccc(N(c2ccccc2)c2cccc3c2[C@@]2(CC3)CCc3cccc(N(c4ccc5c(c4)oc4ccccc45)c4cccc5ccccc45)c32)cc1 ; c1ccc(N(c2ccc3ccccc3c2)c2cccc3c2[C@@]2(CC3)CCc3cccc(N(c4ccccc4)c4ccc5c(c4)oc4ccccc45)c32)cc1 ; c1ccc(N(c2ccc3c(c2)oc2ccccc23)c2cccc3c2[C@@]2(CC3)CCc3cccc(N(c4ccccc4)c4cccc5ccccc45)c32)cc1]

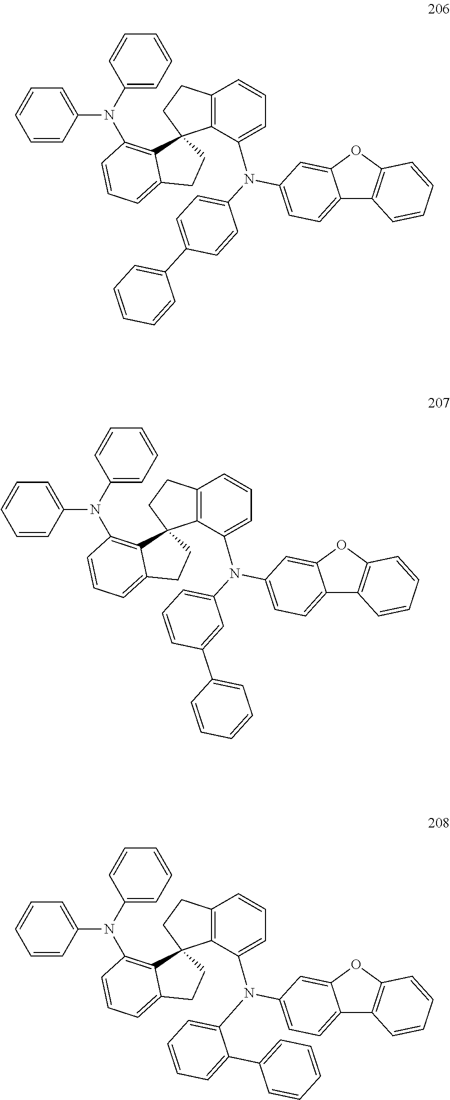 [SMILES: c1ccc(-c2ccc(N(c3ccc4c(c3)oc3ccccc34)c3cccc4c3[C@]3(CCc5cccc(N(c6ccccc6)c6ccccc6)c53)CC4)cc2)cc1 ; c1ccc(-c2cccc(N(c3ccc4c(c3)oc3ccccc34)c3cccc4c3[C@]3(CCc5cccc(N(c6ccccc6)c6ccccc6)c53)CC4)c2)cc1 ; c1ccc(-c2ccccc2N(c2ccc3c(c2)oc2ccccc23)c2cccc3c2[C@]2(CCc4cccc(N(c5ccccc5)c5ccccc5)c42)CC3)cc1]

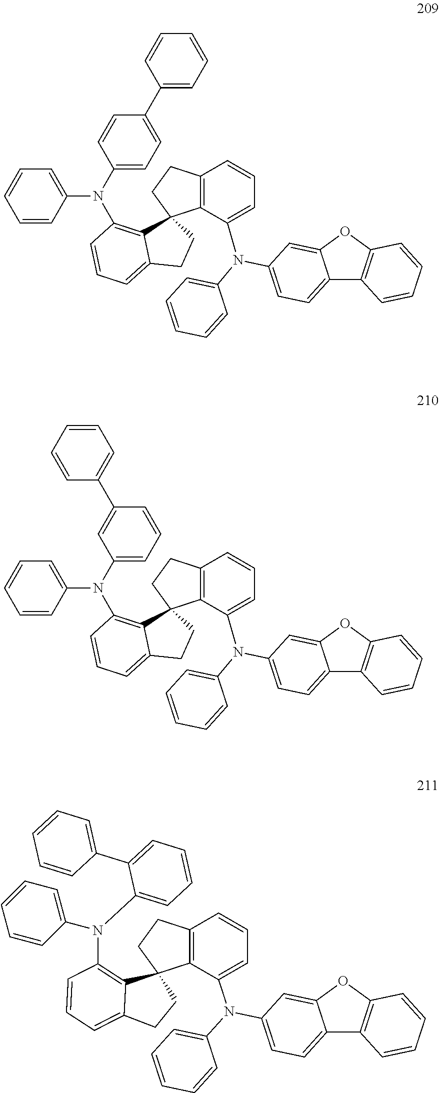 [SMILES: c1ccc(-c2ccc(N(c3ccccc3)c3cccc4c3[C@@]3(CC4)CCc4cccc(N(c5ccccc5)c5ccc6c(c5)oc5ccccc56)c43)cc2)cc1 ; c1ccc(-c2cccc(N(c3ccccc3)c3cccc4c3[C@@]3(CC4)CCc4cccc(N(c5ccccc5)c5ccc6c(c5)oc5ccccc56)c43)c2)cc1 ; c1ccc(-c2ccccc2N(c2ccccc2)c2cccc3c2[C@]2(CCc4cccc(N(c5ccccc5)c5ccc6c(c5)oc5ccccc56)c42)CC3)cc1]

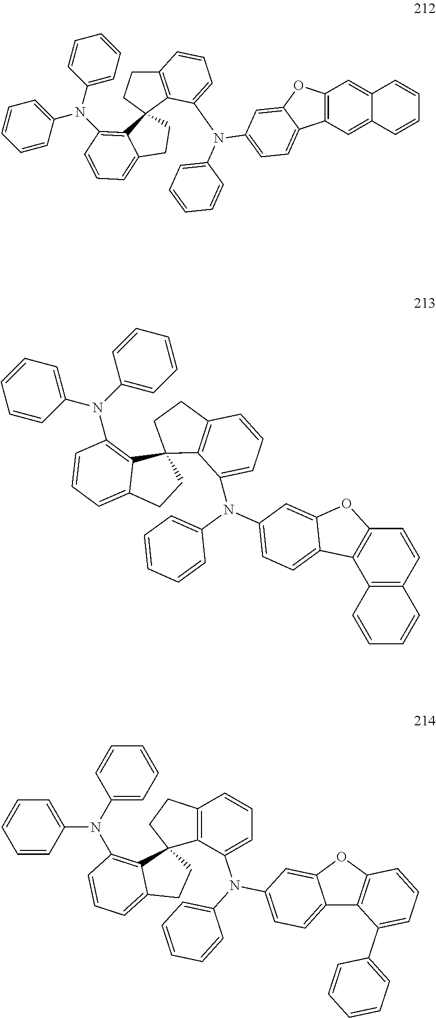 [SMILES: c1ccc(N(c2ccccc2)c2cccc3c2[C@@]2(CC3)CCc3cccc(N(c4ccccc4)c4ccc5c(c4)oc4cc6ccccc6cc45)c32)cc1 ; c1ccc(N(c2ccccc2)c2cccc3c2[C@@]2(CC3)CCc3cccc(N(c4ccccc4)c4ccc5c(c4)oc4ccc6ccccc6c45)c32)cc1 ; c1ccc(-c2cccc3oc4cc(N(c5ccccc5)c5cccc6c5[C@]5(CCc7cccc(N(c8ccccc8)c8ccccc8)c75)CC6)ccc4c23)cc1]

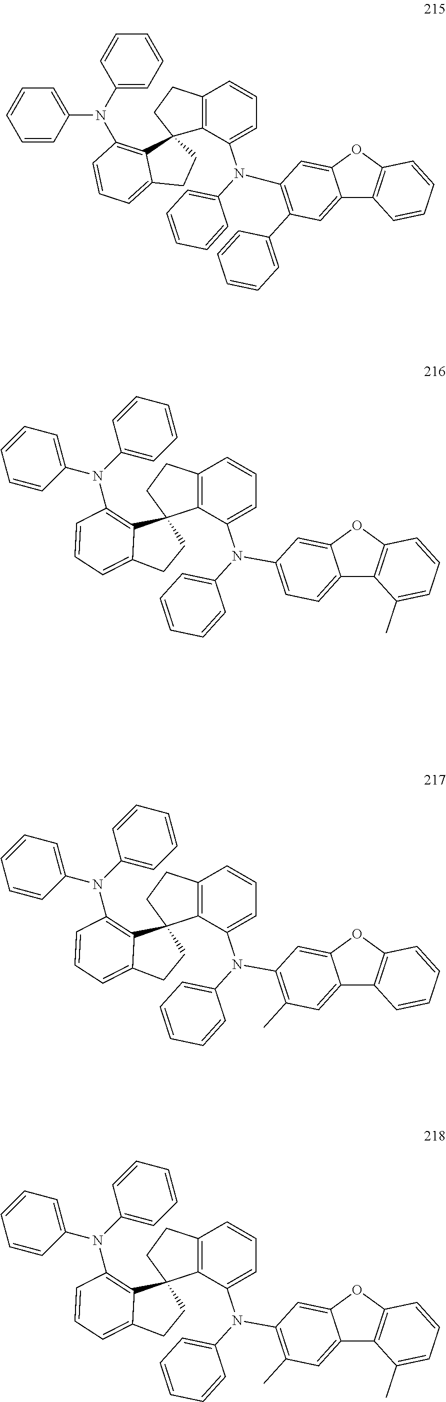 [SMILES: c1ccc(-c2cc3c(cc2N(c2ccccc2)c2cccc4c2[C@]2(CCc5cccc(N(c6ccccc6)c6ccccc6)c52)CC4)oc2ccccc23)cc1 ; Cc1cccc2oc3cc(N(c4ccccc4)c4cccc5c4[C@]4(CCc6cccc(N(c7ccccc7)c7ccccc7)c64)CC5)ccc3c12 ; Cc1cc2c(cc1N(c1ccccc1)c1cccc3c1[C@]1(CCc4cccc(N(c5ccccc5)c5ccccc5)c41)CC3)oc1ccccc12 ; Cc1cc2c(cc1N(c1ccccc1)c1cccc3c1[C@]1(CCc4cccc(N(c5ccccc5)c5ccccc5)c41)CC3)oc1cccc(C)c12]

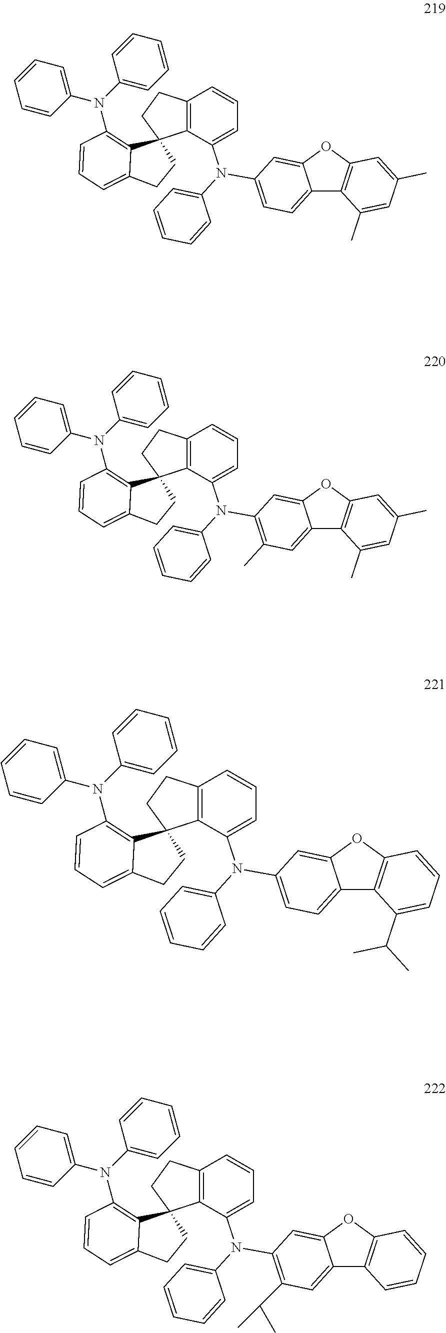 [SMILES: Cc1cc(C)c2c(c1)oc1cc(N(c3ccccc3)c3cccc4c3[C@]3(CCc5cccc(N(c6ccccc6)c6ccccc6)c53)CC4)ccc12 ; Cc1cc(C)c2c(c1)oc1cc(N(c3ccccc3)c3cccc4c3[C@]3(CCc5cccc(N(c6ccccc6)c6ccccc6)c53)CC4)c(C)cc12 ; CC(C)c1cccc2oc3cc(N(c4ccccc4)c4cccc5c4[C@]4(CCc6cccc(N(c7ccccc7)c7ccccc7)c64)CC5)ccc3c12 ; CC(C)c1cc2c(cc1N(c1ccccc1)c1cccc3c1[C@]1(CCc4cccc(N(c5ccccc5)c5ccccc5)c41)CC3)oc1ccccc12]

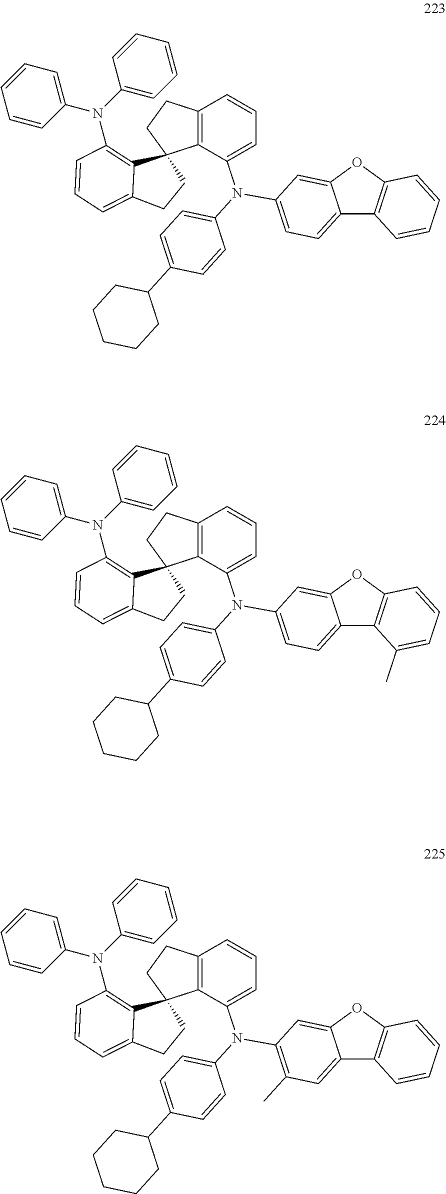 [SMILES: c1ccc(N(c2ccccc2)c2cccc3c2[C@@]2(CC3)CCc3cccc(N(c4ccc(C5CCCCC5)cc4)c4ccc5c(c4)oc4ccccc45)c32)cc1 ; Cc1cccc2oc3cc(N(c4ccc(C5CCCCC5)cc4)c4cccc5c4[C@]4(CCc6cccc(N(c7ccccc7)c7ccccc7)c64)CC5)ccc3c12 ; Cc1cc2c(cc1N(c1ccc(C3CCCCC3)cc1)c1cccc3c1[C@]1(CCc4cccc(N(c5ccccc5)c5ccccc5)c41)CC3)oc1ccccc12]

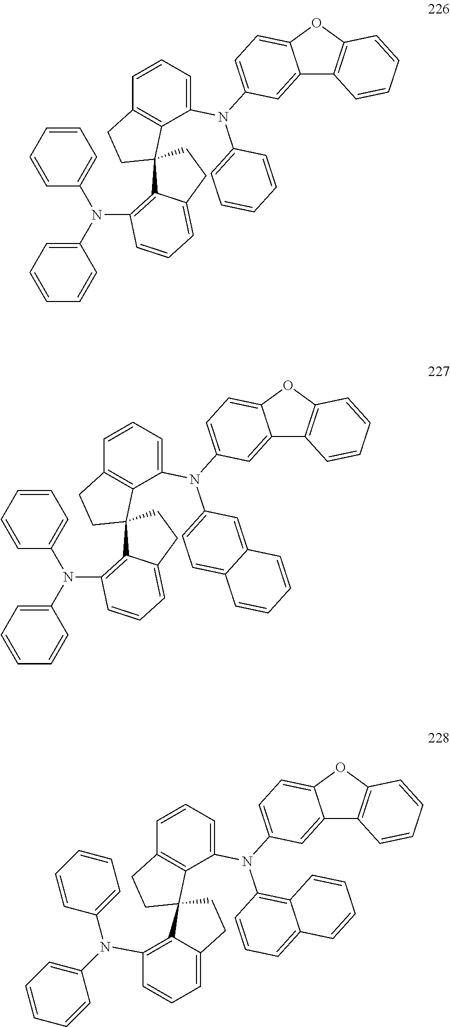 [SMILES: c1ccc(N(c2ccccc2)c2cccc3c2[C@@]2(CC3)CCc3cccc(N(c4ccccc4)c4ccc5oc6ccccc6c5c4)c32)cc1 ; c1ccc(N(c2ccccc2)c2cccc3c2[C@@]2(CC3)CCc3cccc(N(c4ccc5ccccc5c4)c4ccc5oc6ccccc6c5c4)c32)cc1 ; c1ccc(N(c2ccccc2)c2cccc3c2[C@@]2(CC3)CCc3cccc(N(c4ccc5oc6ccccc6c5c4)c4cccc5ccccc45)c32)cc1]

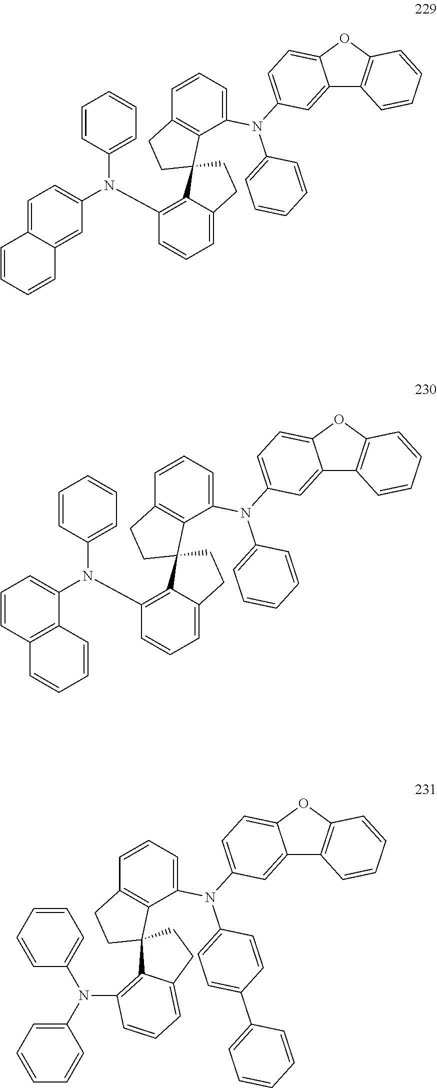 [SMILES: c1ccc(N(c2ccc3ccccc3c2)c2cccc3c2[C@@]2(CC3)CCc3cccc(N(c4ccccc4)c4ccc5oc6ccccc6c5c4)c32)cc1 ; c1ccc(N(c2ccc3oc4ccccc4c3c2)c2cccc3c2[C@@]2(CC3)CCc3cccc(N(c4ccccc4)c4cccc5ccccc45)c32)cc1 ; c1ccc(-c2ccc(N(c3ccc4oc5ccccc5c4c3)c3cccc4c3[C@]3(CCc5cccc(N(c6ccccc6)c6ccccc6)c53)CC4)cc2)cc1]

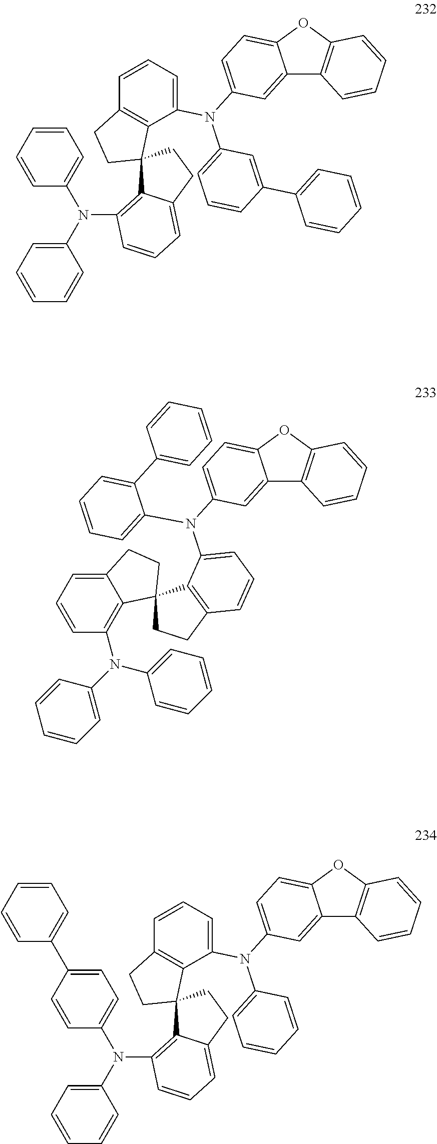 [SMILES: c1ccc(-c2cccc(N(c3ccc4oc5ccccc5c4c3)c3cccc4c3[C@]3(CCc5cccc(N(c6ccccc6)c6ccccc6)c53)CC4)c2)cc1 ; c1ccc(-c2ccccc2N(c2ccc3oc4ccccc4c3c2)c2cccc3c2[C@]2(CCc4cccc(N(c5ccccc5)c5ccccc5)c42)CC3)cc1 ; c1ccc(-c2ccc(N(c3ccccc3)c3cccc4c3[C@@]3(CC4)CCc4cccc(N(c5ccccc5)c5ccc6oc7ccccc7c6c5)c43)cc2)cc1]

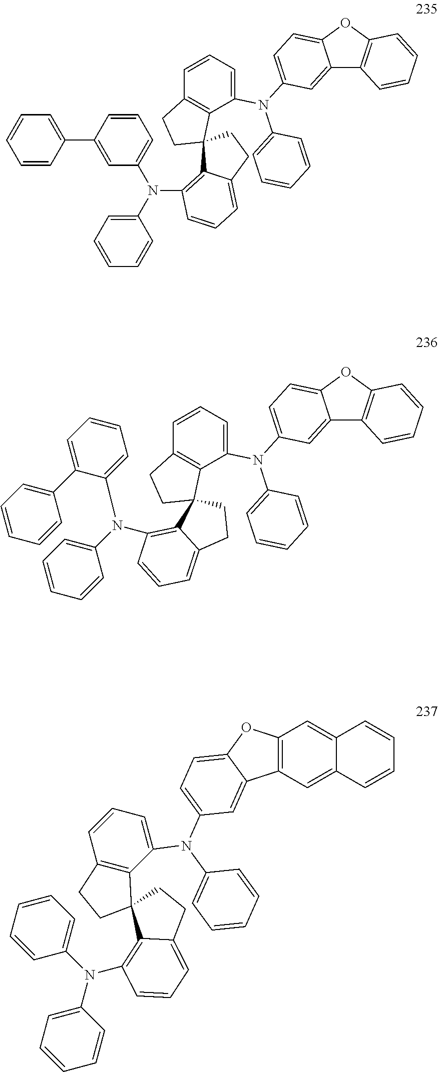 [SMILES: c1ccc(-c2cccc(N(c3ccccc3)c3cccc4c3[C@@]3(CC4)CCc4cccc(N(c5ccccc5)c5ccc6oc7ccccc7c6c5)c43)c2)cc1 ; c1ccc(-c2ccccc2N(c2ccccc2)c2cccc3c2[C@]2(CCc4cccc(N(c5ccccc5)c5ccc6oc7ccccc7c6c5)c42)CC3)cc1 ; c1ccc(N(c2ccccc2)c2cccc3c2[C@@]2(CC3)CCc3cccc(N(c4ccccc4)c4ccc5oc6cc7ccccc7cc6c5c4)c32)cc1]

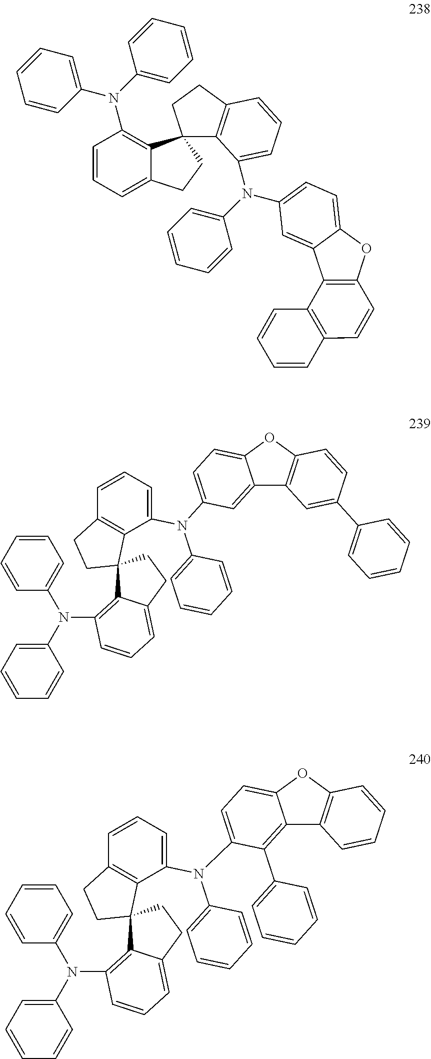 [SMILES: c1ccc(N(c2ccccc2)c2cccc3c2[C@@]2(CC3)CCc3cccc(N(c4ccccc4)c4ccc5oc6ccc7ccccc7c6c5c4)c32)cc1 ; c1ccc(-c2ccc3oc4ccc(N(c5ccccc5)c5cccc6c5[C@]5(CCc7cccc(N(c8ccccc8)c8ccccc8)c75)CC6)cc4c3c2)cc1 ; c1ccc(-c2c(N(c3ccccc3)c3cccc4c3[C@]3(CCc5cccc(N(c6ccccc6)c6ccccc6)c53)CC4)ccc3oc4ccccc4c23)cc1]

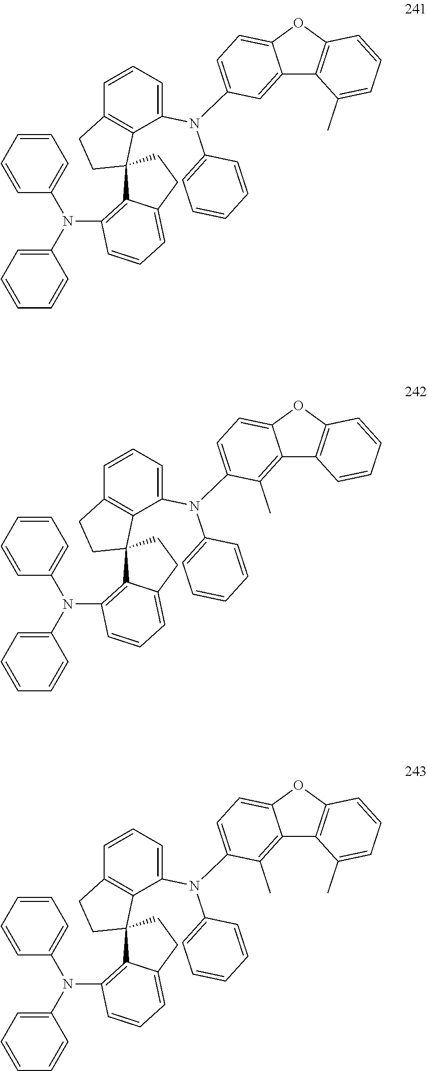 [SMILES: Cc1cccc2oc3ccc(N(c4ccccc4)c4cccc5c4[C@]4(CCc6cccc(N(c7ccccc7)c7ccccc7)c64)CC5)cc3c12 ; Cc1c(N(c2ccccc2)c2cccc3c2[C@]2(CCc4cccc(N(c5ccccc5)c5ccccc5)c42)CC3)ccc2oc3ccccc3c12 ; Cc1cccc2oc3ccc(N(c4ccccc4)c4cccc5c4[C@]4(CCc6cccc(N(c7ccccc7)c7ccccc7)c64)CC5)c(C)c3c12]

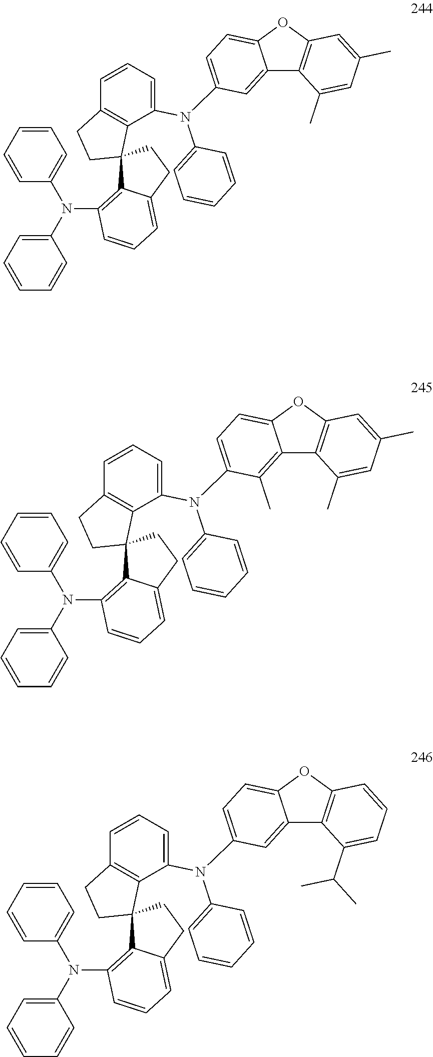 [SMILES: Cc1cc(C)c2c(c1)oc1ccc(N(c3ccccc3)c3cccc4c3[C@]3(CCc5cccc(N(c6ccccc6)c6ccccc6)c53)CC4)cc12 ; Cc1cc(C)c2c(c1)oc1ccc(N(c3ccccc3)c3cccc4c3[C@]3(CCc5cccc(N(c6ccccc6)c6ccccc6)c53)CC4)c(C)c12 ; CC(C)c1cccc2oc3ccc(N(c4ccccc4)c4cccc5c4[C@]4(CCc6cccc(N(c7ccccc7)c7ccccc7)c64)CC5)cc3c12]

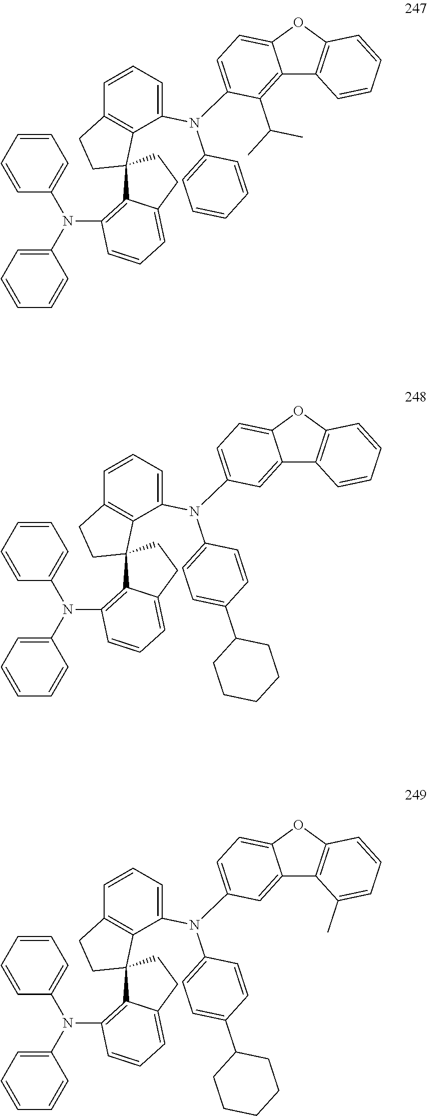 [SMILES: CC(C)c1c(N(c2ccccc2)c2cccc3c2[C@]2(CCc4cccc(N(c5ccccc5)c5ccccc5)c42)CC3)ccc2oc3ccccc3c12 ; c1ccc(N(c2ccccc2)c2cccc3c2[C@@]2(CC3)CCc3cccc(N(c4ccc(C5CCCCC5)cc4)c4ccc5oc6ccccc6c5c4)c32)cc1 ; Cc1cccc2oc3ccc(N(c4ccc(C5CCCCC5)cc4)c4cccc5c4[C@]4(CCc6cccc(N(c7ccccc7)c7ccccc7)c64)CC5)cc3c12]

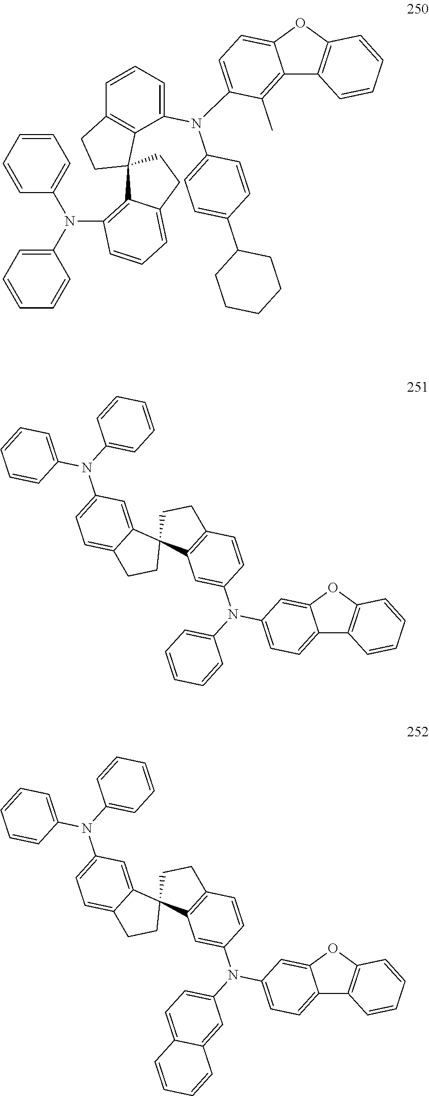 [SMILES: Cc1c(N(c2ccc(C3CCCCC3)cc2)c2cccc3c2[C@]2(CCc4cccc(N(c5ccccc5)c5ccccc5)c42)CC3)ccc2oc3ccccc3c12 ; c1ccc(N(c2ccccc2)c2ccc3c(c2)[C@@]2(CC3)CCc3ccc(N(c4ccccc4)c4ccc5c(c4)oc4ccccc45)cc32)cc1 ; c1ccc(N(c2ccccc2)c2ccc3c(c2)[C@@]2(CC3)CCc3ccc(N(c4ccc5ccccc5c4)c4ccc5c(c4)oc4ccccc45)cc32)cc1]

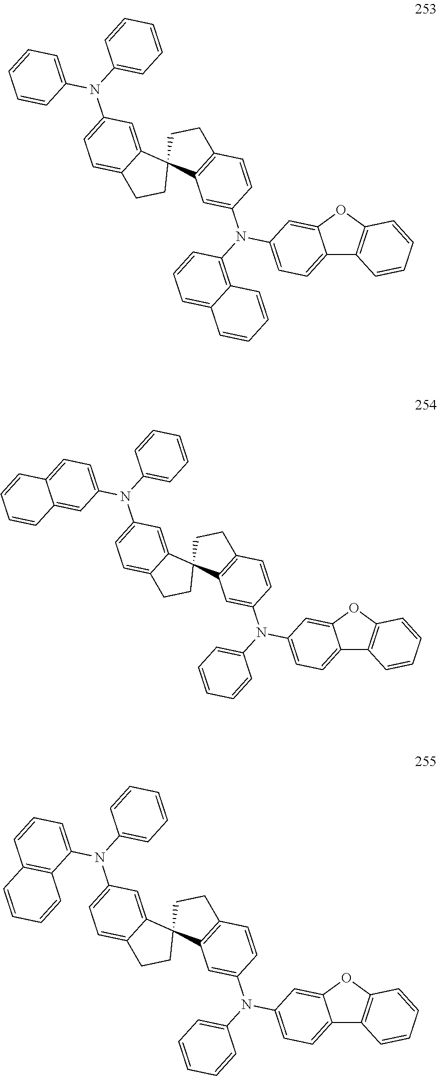 [SMILES: c1ccc(N(c2ccccc2)c2ccc3c(c2)[C@@]2(CC3)CCc3ccc(N(c4ccc5c(c4)oc4ccccc45)c4cccc5ccccc45)cc32)cc1 ; c1ccc(N(c2ccc3c(c2)[C@@]2(CC3)CCc3ccc(N(c4ccccc4)c4ccc5c(c4)oc4ccccc45)cc32)c2ccc3ccccc3c2)cc1 ; c1ccc(N(c2ccc3c(c2)[C@@]2(CC3)CCc3ccc(N(c4ccccc4)c4cccc5ccccc45)cc32)c2ccc3c(c2)oc2ccccc23)cc1]

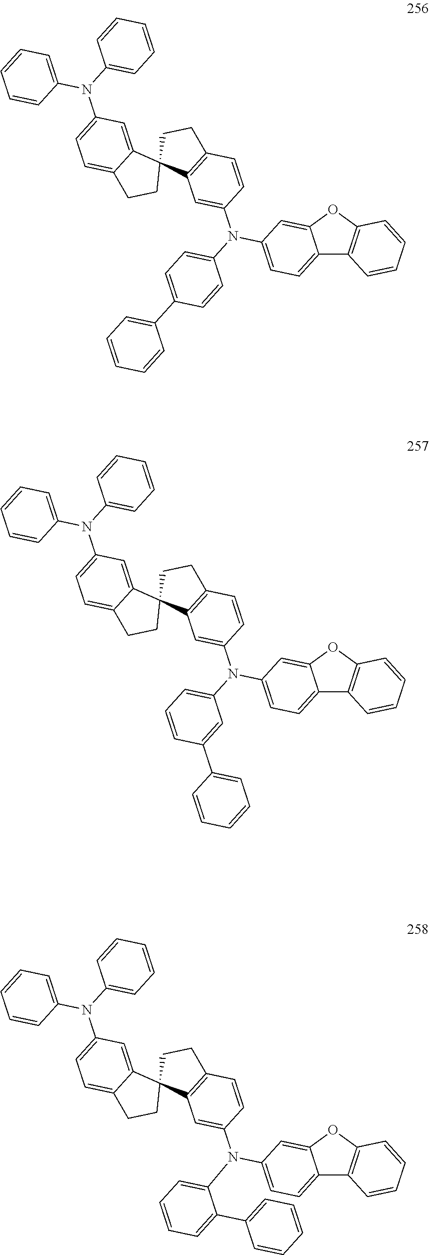 [SMILES: c1ccc(-c2ccc(N(c3ccc4c(c3)[C@]3(CCc5ccc(N(c6ccccc6)c6ccccc6)cc53)CC4)c3ccc4c(c3)oc3ccccc34)cc2)cc1 ; c1ccc(-c2cccc(N(c3ccc4c(c3)[C@]3(CCc5ccc(N(c6ccccc6)c6ccccc6)cc53)CC4)c3ccc4c(c3)oc3ccccc34)c2)cc1 ; c1ccc(-c2ccccc2N(c2ccc3c(c2)[C@]2(CCc4ccc(N(c5ccccc5)c5ccccc5)cc42)CC3)c2ccc3c(c2)oc2ccccc23)cc1]

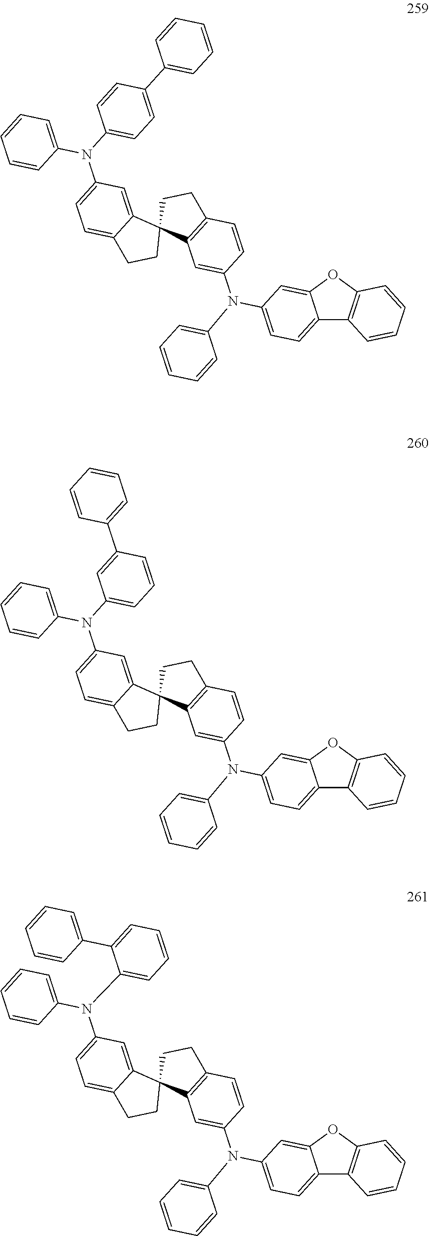 [SMILES: c1ccc(-c2ccc(N(c3ccccc3)c3ccc4c(c3)[C@@]3(CC4)CCc4ccc(N(c5ccccc5)c5ccc6c(c5)oc5ccccc56)cc43)cc2)cc1 ; c1ccc(-c2cccc(N(c3ccccc3)c3ccc4c(c3)[C@@]3(CC4)CCc4ccc(N(c5ccccc5)c5ccc6c(c5)oc5ccccc56)cc43)c2)cc1 ; c1ccc(-c2ccccc2N(c2ccccc2)c2ccc3c(c2)[C@]2(CCc4ccc(N(c5ccccc5)c5ccc6c(c5)oc5ccccc56)cc42)CC3)cc1]

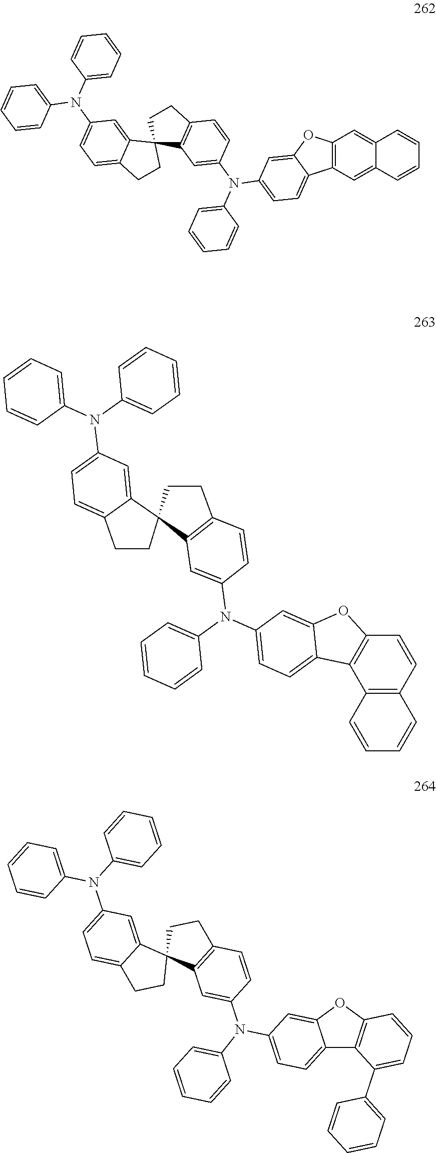 [SMILES: c1ccc(N(c2ccccc2)c2ccc3c(c2)[C@@]2(CC3)CCc3ccc(N(c4ccccc4)c4ccc5c(c4)oc4cc6ccccc6cc45)cc32)cc1 ; c1ccc(N(c2ccccc2)c2ccc3c(c2)[C@@]2(CC3)CCc3ccc(N(c4ccccc4)c4ccc5c(c4)oc4ccc6ccccc6c45)cc32)cc1 ; c1ccc(-c2cccc3oc4cc(N(c5ccccc5)c5ccc6c(c5)[C@]5(CCc7ccc(N(c8ccccc8)c8ccccc8)cc75)CC6)ccc4c23)cc1]

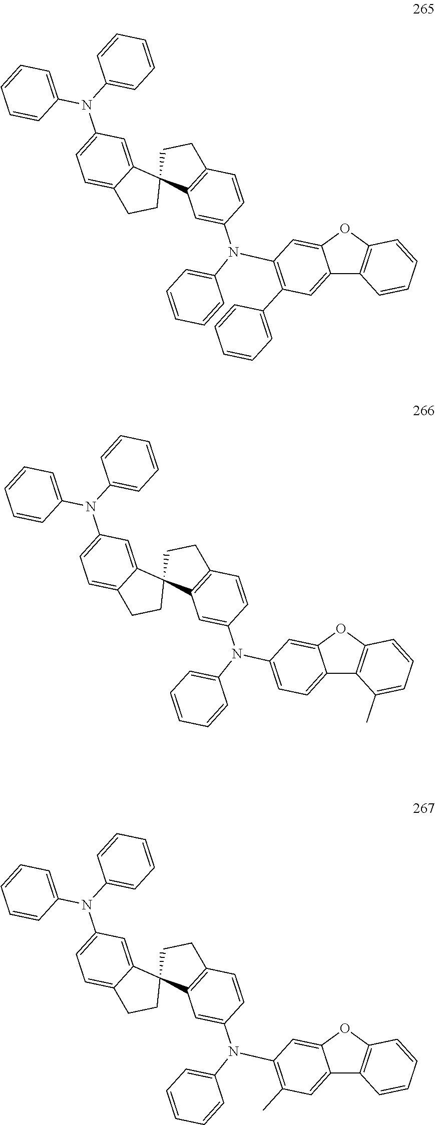 [SMILES: c1ccc(-c2cc3c(cc2N(c2ccccc2)c2ccc4c(c2)[C@]2(CCc5ccc(N(c6ccccc6)c6ccccc6)cc52)CC4)oc2ccccc23)cc1 ; Cc1cccc2oc3cc(N(c4ccccc4)c4ccc5c(c4)[C@]4(CCc6ccc(N(c7ccccc7)c7ccccc7)cc64)CC5)ccc3c12 ; Cc1cc2c(cc1N(c1ccccc1)c1ccc3c(c1)[C@]1(CCc4ccc(N(c5ccccc5)c5ccccc5)cc41)CC3)oc1ccccc12]

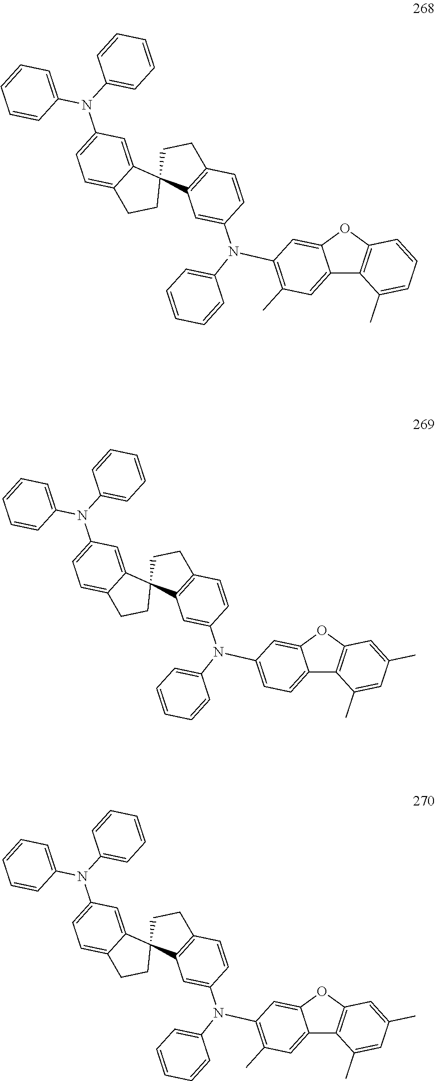 [SMILES: Cc1cc2c(cc1N(c1ccccc1)c1ccc3c(c1)[C@]1(CCc4ccc(N(c5ccccc5)c5ccccc5)cc41)CC3)oc1cccc(C)c12 ; Cc1cc(C)c2c(c1)oc1cc(N(c3ccccc3)c3ccc4c(c3)[C@]3(CCc5ccc(N(c6ccccc6)c6ccccc6)cc53)CC4)ccc12 ; Cc1cc(C)c2c(c1)oc1cc(N(c3ccccc3)c3ccc4c(c3)[C@]3(CCc5ccc(N(c6ccccc6)c6ccccc6)cc53)CC4)c(C)cc12]

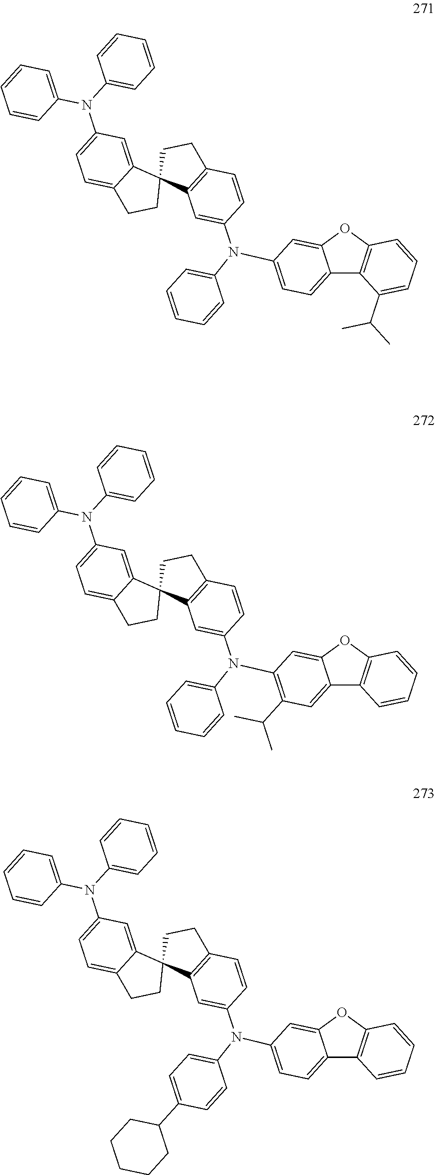 [SMILES: CC(C)c1cccc2oc3cc(N(c4ccccc4)c4ccc5c(c4)[C@]4(CCc6ccc(N(c7ccccc7)c7ccccc7)cc64)CC5)ccc3c12 ; CC(C)c1cc2c(cc1N(c1ccccc1)c1ccc3c(c1)[C@]1(CCc4ccc(N(c5ccccc5)c5ccccc5)cc41)CC3)oc1ccccc12 ; c1ccc(N(c2ccccc2)c2ccc3c(c2)[C@@]2(CC3)CCc3ccc(N(c4ccc(C5CCCCC5)cc4)c4ccc5c(c4)oc4ccccc45)cc32)cc1]

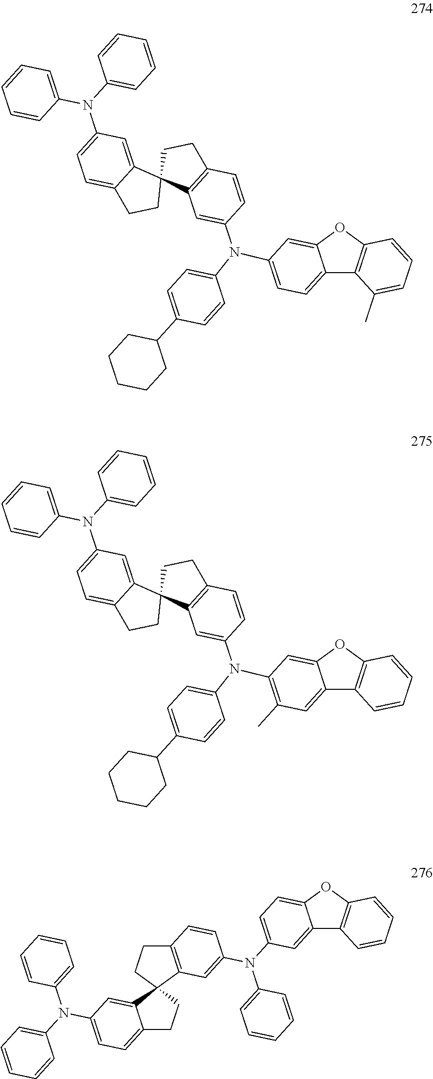 [SMILES: Cc1cccc2oc3cc(N(c4ccc(C5CCCCC5)cc4)c4ccc5c(c4)[C@]4(CCc6ccc(N(c7ccccc7)c7ccccc7)cc64)CC5)ccc3c12 ; Cc1cc2c(cc1N(c1ccc(C3CCCCC3)cc1)c1ccc3c(c1)[C@]1(CCc4ccc(N(c5ccccc5)c5ccccc5)cc41)CC3)oc1ccccc12 ; c1ccc(N(c2ccccc2)c2ccc3c(c2)[C@@]2(CC3)CCc3ccc(N(c4ccccc4)c4ccc5oc6ccccc6c5c4)cc32)cc1]

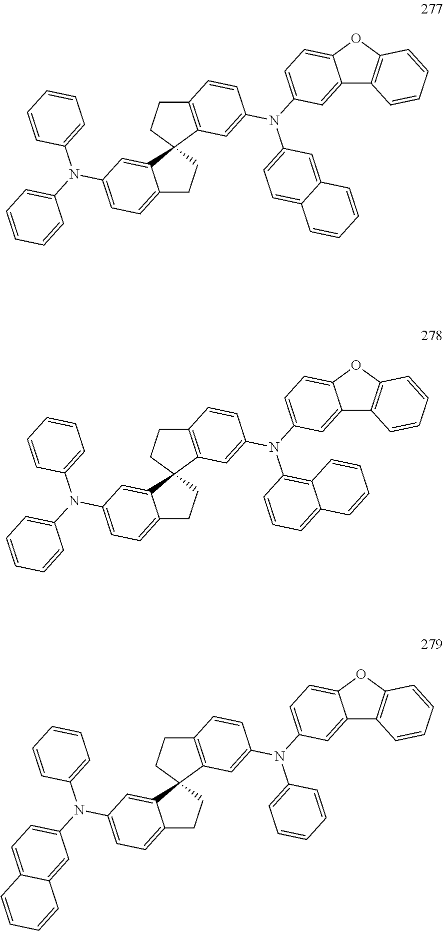 [SMILES: c1ccc(N(c2ccccc2)c2ccc3c(c2)[C@@]2(CC3)CCc3ccc(N(c4ccc5ccccc5c4)c4ccc5oc6ccccc6c5c4)cc32)cc1 ; c1ccc(N(c2ccccc2)c2ccc3c(c2)[C@@]2(CC3)CCc3ccc(N(c4ccc5oc6ccccc6c5c4)c4cccc5ccccc45)cc32)cc1 ; c1ccc(N(c2ccc3c(c2)[C@@]2(CC3)CCc3ccc(N(c4ccccc4)c4ccc5oc6ccccc6c5c4)cc32)c2ccc3ccccc3c2)cc1]

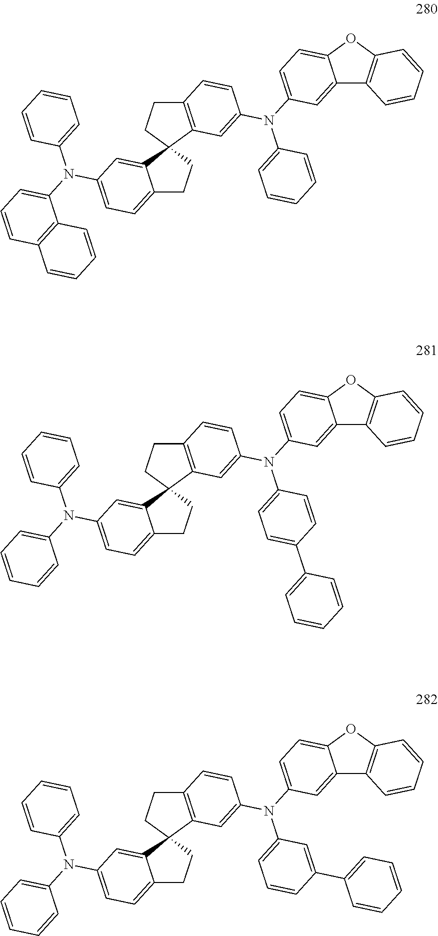 [SMILES: c1ccc(N(c2ccc3c(c2)[C@@]2(CC3)CCc3ccc(N(c4ccccc4)c4cccc5ccccc45)cc32)c2ccc3oc4ccccc4c3c2)cc1 ; c1ccc(-c2ccc(N(c3ccc4c(c3)[C@]3(CCc5ccc(N(c6ccccc6)c6ccccc6)cc53)CC4)c3ccc4oc5ccccc5c4c3)cc2)cc1 ; c1ccc(-c2cccc(N(c3ccc4c(c3)[C@]3(CCc5ccc(N(c6ccccc6)c6ccccc6)cc53)CC4)c3ccc4oc5ccccc5c4c3)c2)cc1]

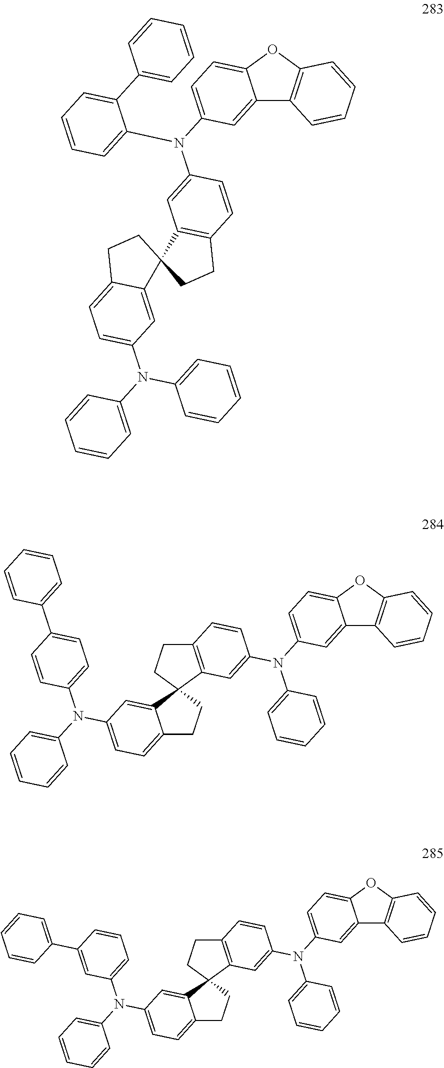 [SMILES: c1ccc(-c2ccccc2N(c2ccc3c(c2)[C@]2(CCc4ccc(N(c5ccccc5)c5ccccc5)cc42)CC3)c2ccc3oc4ccccc4c3c2)cc1 ; c1ccc(-c2ccc(N(c3ccccc3)c3ccc4c(c3)[C@@]3(CC4)CCc4ccc(N(c5ccccc5)c5ccc6oc7ccccc7c6c5)cc43)cc2)cc1 ; c1ccc(-c2cccc(N(c3ccccc3)c3ccc4c(c3)[C@@]3(CC4)CCc4ccc(N(c5ccccc5)c5ccc6oc7ccccc7c6c5)cc43)c2)cc1]

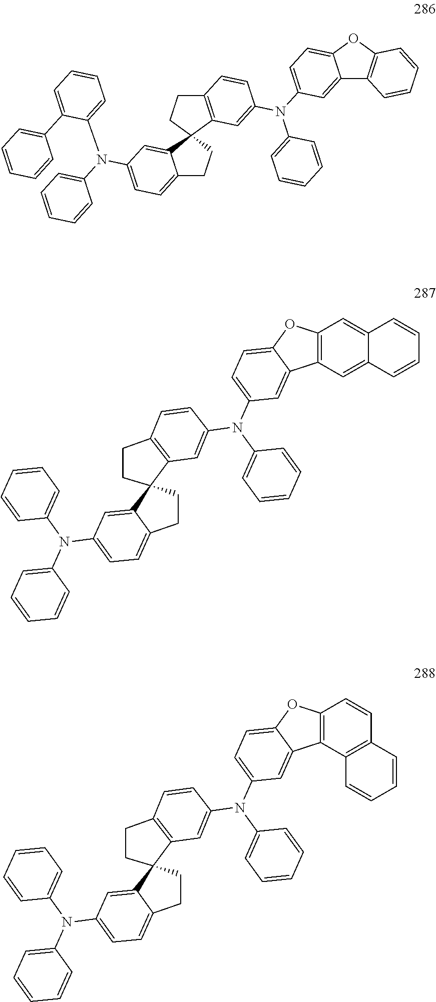 [SMILES: c1ccc(-c2ccccc2N(c2ccccc2)c2ccc3c(c2)[C@]2(CCc4ccc(N(c5ccccc5)c5ccc6oc7ccccc7c6c5)cc42)CC3)cc1 ; c1ccc(N(c2ccccc2)c2ccc3c(c2)[C@@]2(CC3)CCc3ccc(N(c4ccccc4)c4ccc5oc6cc7ccccc7cc6c5c4)cc32)cc1 ; c1ccc(N(c2ccccc2)c2ccc3c(c2)[C@@]2(CC3)CCc3ccc(N(c4ccccc4)c4ccc5oc6ccc7ccccc7c6c5c4)cc32)cc1]

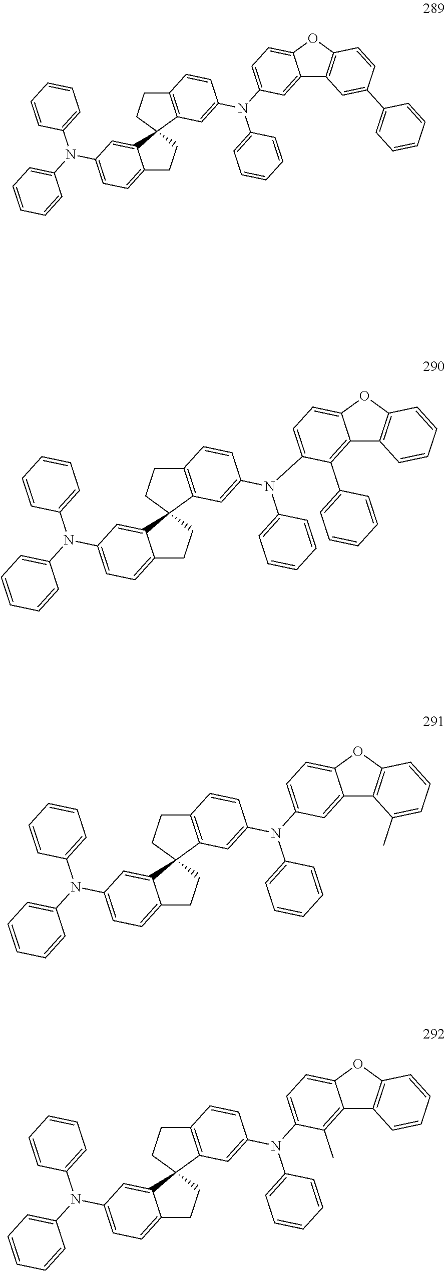 [SMILES: c1ccc(-c2ccc3oc4ccc(N(c5ccccc5)c5ccc6c(c5)[C@]5(CCc7ccc(N(c8ccccc8)c8ccccc8)cc75)CC6)cc4c3c2)cc1 ; c1ccc(-c2c(N(c3ccccc3)c3ccc4c(c3)[C@]3(CCc5ccc(N(c6ccccc6)c6ccccc6)cc53)CC4)ccc3oc4ccccc4c23)cc1 ; Cc1cccc2oc3ccc(N(c4ccccc4)c4ccc5c(c4)[C@]4(CCc6ccc(N(c7ccccc7)c7ccccc7)cc64)CC5)cc3c12 ; Cc1c(N(c2ccccc2)c2ccc3c(c2)[C@]2(CCc4ccc(N(c5ccccc5)c5ccccc5)cc42)CC3)ccc2oc3ccccc3c12]

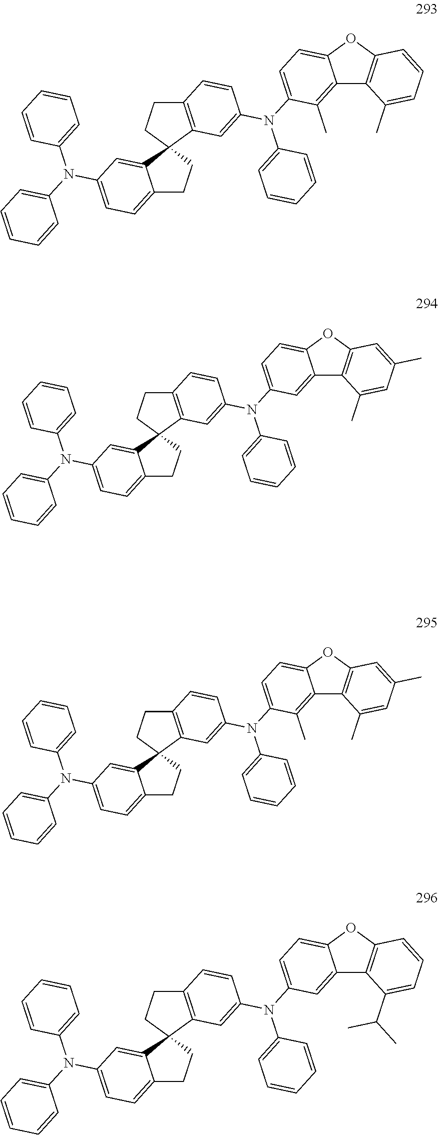 [SMILES: Cc1cccc2oc3ccc(N(c4ccccc4)c4ccc5c(c4)[C@]4(CCc6ccc(N(c7ccccc7)c7ccccc7)cc64)CC5)c(C)c3c12 ; Cc1cc(C)c2c(c1)oc1ccc(N(c3ccccc3)c3ccc4c(c3)[C@]3(CCc5ccc(N(c6ccccc6)c6ccccc6)cc53)CC4)cc12 ; Cc1cc(C)c2c(c1)oc1ccc(N(c3ccccc3)c3ccc4c(c3)[C@]3(CCc5ccc(N(c6ccccc6)c6ccccc6)cc53)CC4)c(C)c12 ; CC(C)c1cccc2oc3ccc(N(c4ccccc4)c4ccc5c(c4)[C@]4(CCc6ccc(N(c7ccccc7)c7ccccc7)cc64)CC5)cc3c12]

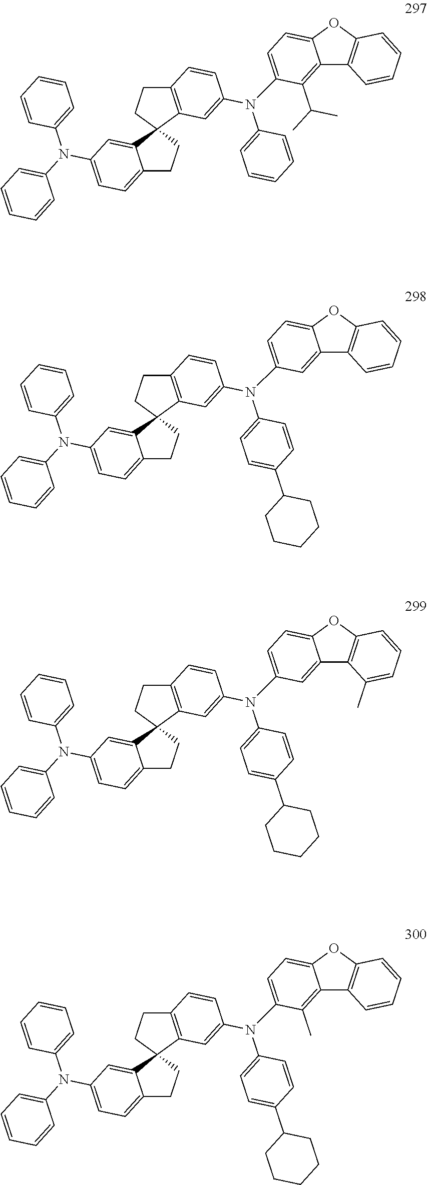 [SMILES: CC(C)c1c(N(c2ccccc2)c2ccc3c(c2)[C@]2(CCc4ccc(N(c5ccccc5)c5ccccc5)cc42)CC3)ccc2oc3ccccc3c12 ; c1ccc(N(c2ccccc2)c2ccc3c(c2)[C@@]2(CC3)CCc3ccc(N(c4ccc(C5CCCCC5)cc4)c4ccc5oc6ccccc6c5c4)cc32)cc1 ; Cc1cccc2oc3ccc(N(c4ccc(C5CCCCC5)cc4)c4ccc5c(c4)[C@]4(CCc6ccc(N(c7ccccc7)c7ccccc7)cc64)CC5)cc3c12 ; Cc1c(N(c2ccc(C3CCCCC3)cc2)c2ccc3c(c2)[C@]2(CCc4ccc(N(c5ccccc5)c5ccccc5)cc42)CC3)ccc2oc3ccccc3c12]

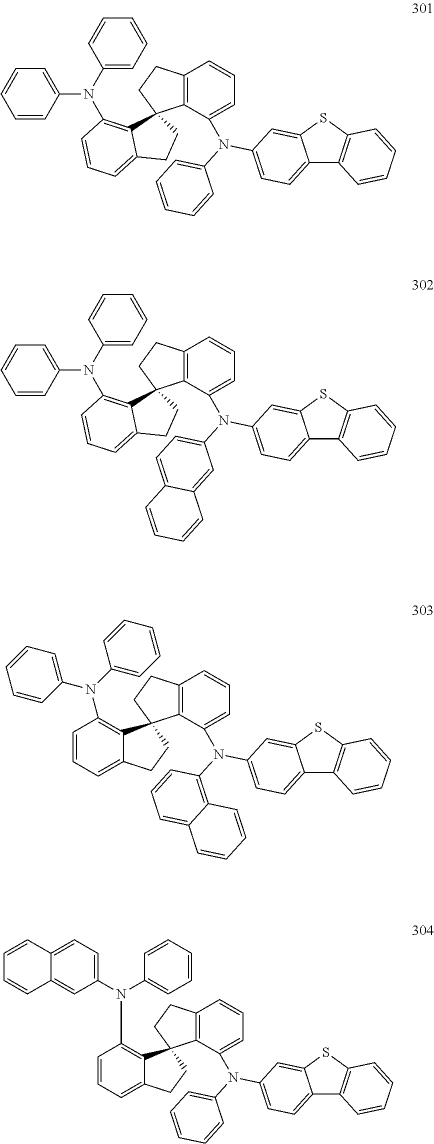 [SMILES: c1ccc(N(c2ccccc2)c2cccc3c2[C@@]2(CC3)CCc3cccc(N(c4ccccc4)c4ccc5c(c4)sc4ccccc45)c32)cc1 ; c1ccc(N(c2ccccc2)c2cccc3c2[C@@]2(CC3)CCc3cccc(N(c4ccc5ccccc5c4)c4ccc5c(c4)sc4ccccc45)c32)cc1 ; c1ccc(N(c2ccccc2)c2cccc3c2[C@@]2(CC3)CCc3cccc(N(c4ccc5c(c4)sc4ccccc45)c4cccc5ccccc45)c32)cc1 ; c1ccc(N(c2ccc3ccccc3c2)c2cccc3c2[C@@]2(CC3)CCc3cccc(N(c4ccccc4)c4ccc5c(c4)sc4ccccc45)c32)cc1]

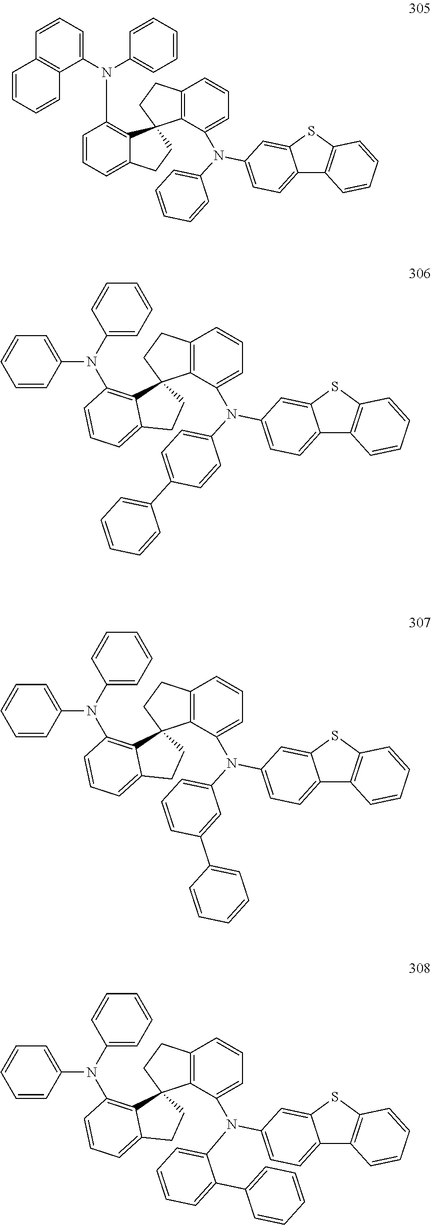 [SMILES: c1ccc(N(c2ccc3c(c2)sc2ccccc23)c2cccc3c2[C@@]2(CC3)CCc3cccc(N(c4ccccc4)c4cccc5ccccc45)c32)cc1 ; c1ccc(-c2ccc(N(c3ccc4c(c3)sc3ccccc34)c3cccc4c3[C@]3(CCc5cccc(N(c6ccccc6)c6ccccc6)c53)CC4)cc2)cc1 ; c1ccc(-c2cccc(N(c3ccc4c(c3)sc3ccccc34)c3cccc4c3[C@]3(CCc5cccc(N(c6ccccc6)c6ccccc6)c53)CC4)c2)cc1 ; c1ccc(-c2ccccc2N(c2ccc3c(c2)sc2ccccc23)c2cccc3c2[C@]2(CCc4cccc(N(c5ccccc5)c5ccccc5)c42)CC3)cc1]

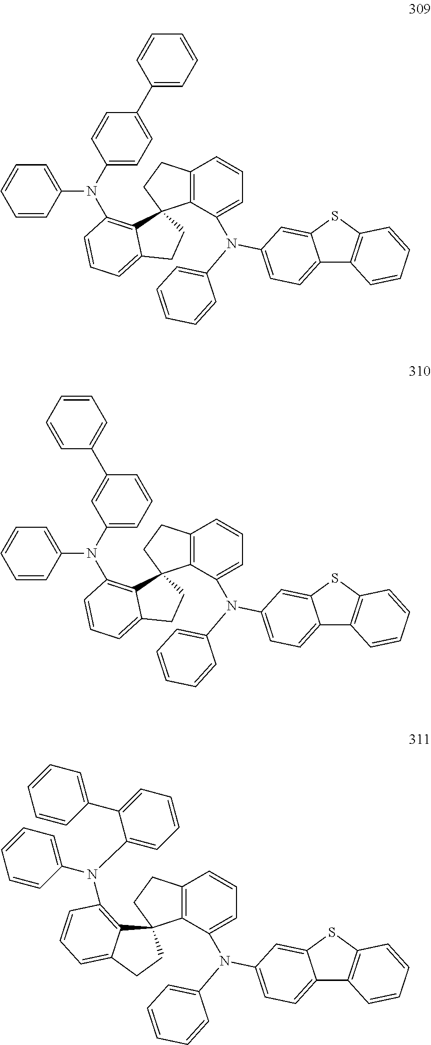 [SMILES: c1ccc(-c2ccc(N(c3ccccc3)c3cccc4c3[C@@]3(CC4)CCc4cccc(N(c5ccccc5)c5ccc6c(c5)sc5ccccc56)c43)cc2)cc1 ; c1ccc(-c2cccc(N(c3ccccc3)c3cccc4c3[C@@]3(CC4)CCc4cccc(N(c5ccccc5)c5ccc6c(c5)sc5ccccc56)c43)c2)cc1 ; c1ccc(-c2ccccc2N(c2ccccc2)c2cccc3c2[C@]2(CCc4cccc(N(c5ccccc5)c5ccc6c(c5)sc5ccccc56)c42)CC3)cc1]

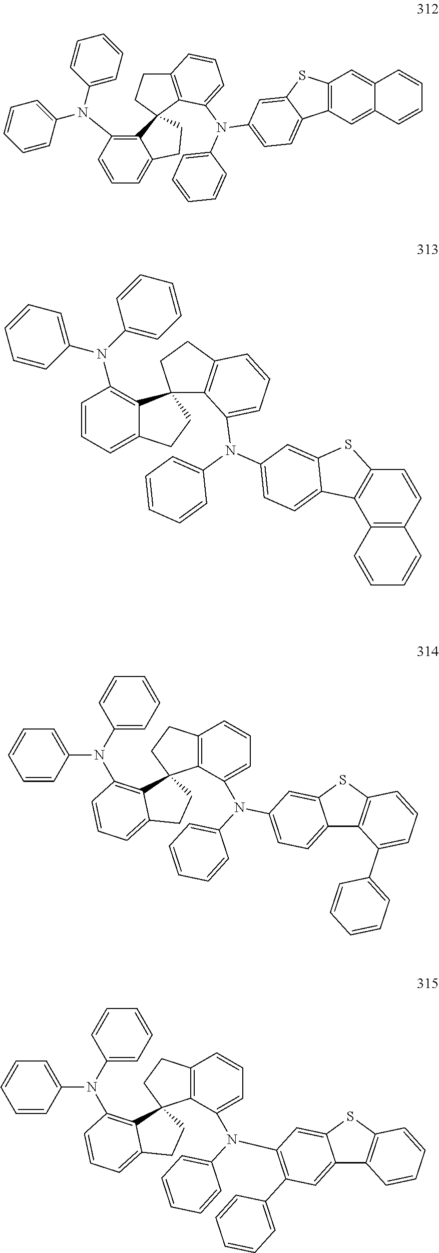 [SMILES: c1ccc(N(c2ccccc2)c2cccc3c2[C@@]2(CC3)CCc3cccc(N(c4ccccc4)c4ccc5c(c4)sc4cc6ccccc6cc45)c32)cc1 ; c1ccc(N(c2ccccc2)c2cccc3c2[C@@]2(CC3)CCc3cccc(N(c4ccccc4)c4ccc5c(c4)sc4ccc6ccccc6c45)c32)cc1 ; c1ccc(-c2cccc3sc4cc(N(c5ccccc5)c5cccc6c5[C@]5(CCc7cccc(N(c8ccccc8)c8ccccc8)c75)CC6)ccc4c23)cc1 ; c1ccc(-c2cc3c(cc2N(c2ccccc2)c2cccc4c2[C@]2(CCc5cccc(N(c6ccccc6)c6ccccc6)c52)CC4)sc2ccccc23)cc1]

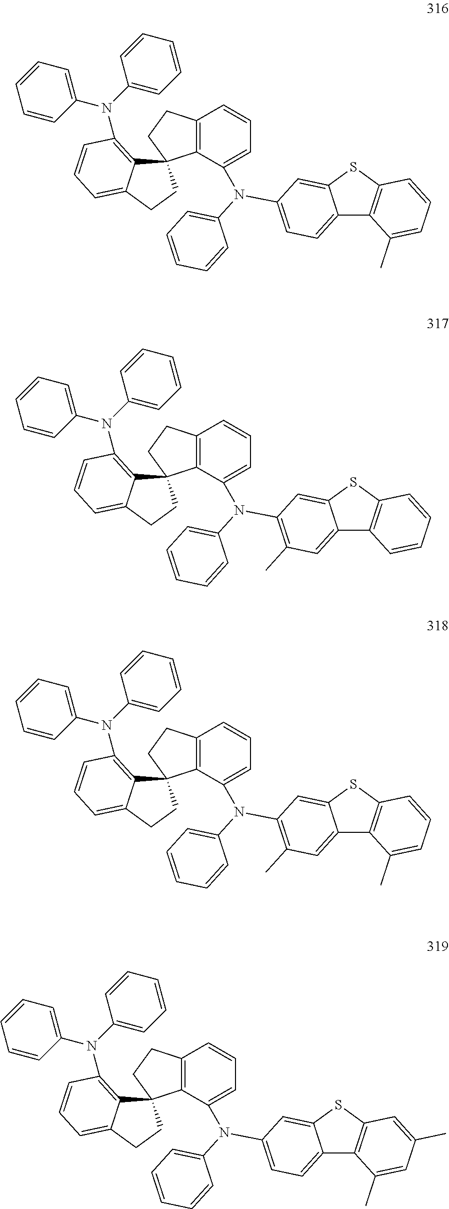 [SMILES: Cc1cccc2sc3cc(N(c4ccccc4)c4cccc5c4[C@]4(CCc6cccc(N(c7ccccc7)c7ccccc7)c64)CC5)ccc3c12 ; Cc1cc2c(cc1N(c1ccccc1)c1cccc3c1[C@]1(CCc4cccc(N(c5ccccc5)c5ccccc5)c41)CC3)sc1ccccc12 ; Cc1cc2c(cc1N(c1ccccc1)c1cccc3c1[C@]1(CCc4cccc(N(c5ccccc5)c5ccccc5)c41)CC3)sc1cccc(C)c12 ; Cc1cc(C)c2c(c1)sc1cc(N(c3ccccc3)c3cccc4c3[C@]3(CCc5cccc(N(c6ccccc6)c6ccccc6)c53)CC4)ccc12]

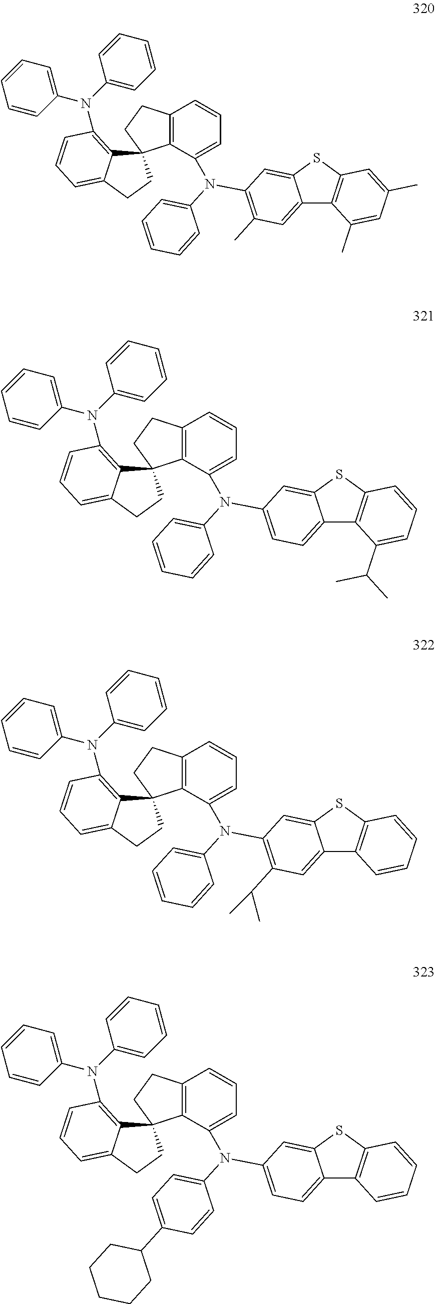 [SMILES: Cc1cc(C)c2c(c1)sc1cc(N(c3ccccc3)c3cccc4c3[C@]3(CCc5cccc(N(c6ccccc6)c6ccccc6)c53)CC4)c(C)cc12 ; CC(C)c1cccc2sc3cc(N(c4ccccc4)c4cccc5c4[C@]4(CCc6cccc(N(c7ccccc7)c7ccccc7)c64)CC5)ccc3c12 ; CC(C)c1cc2c(cc1N(c1ccccc1)c1cccc3c1[C@]1(CCc4cccc(N(c5ccccc5)c5ccccc5)c41)CC3)sc1ccccc12 ; c1ccc(N(c2ccccc2)c2cccc3c2[C@@]2(CC3)CCc3cccc(N(c4ccc(C5CCCCC5)cc4)c4ccc5c(c4)sc4ccccc45)c32)cc1]

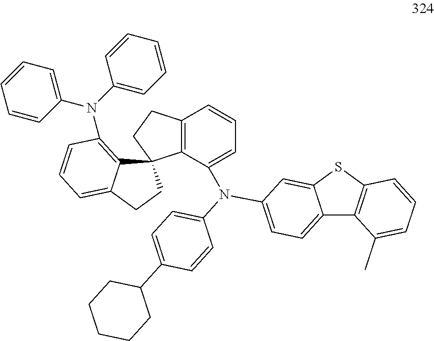 [SMILES: Cc1cccc2sc3cc(N(c4ccc(C5CCCCC5)cc4)c4cccc5c4[C@]4(CCc6cccc(N(c7ccccc7)c7ccccc7)c64)CC5)ccc3c12]

[chemical structure]

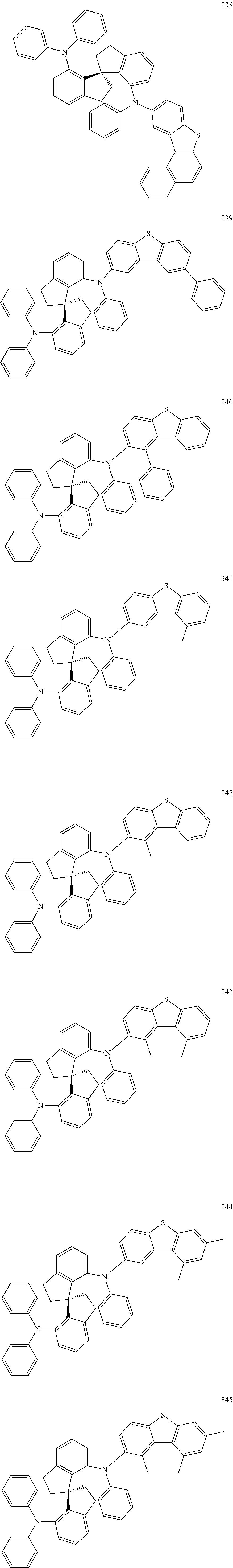

[chemical structure]

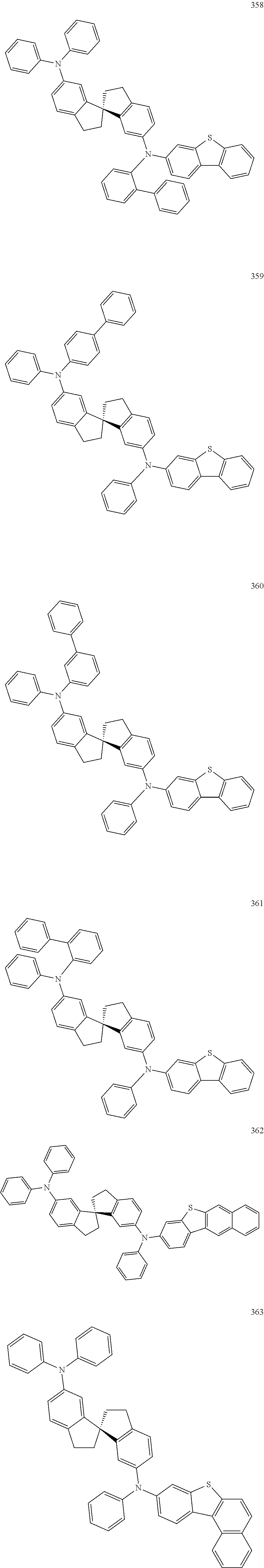

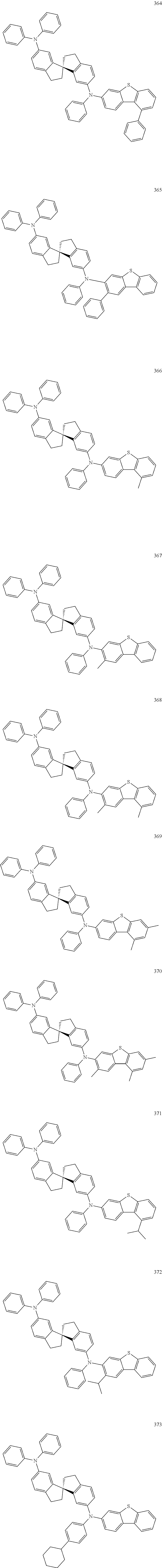

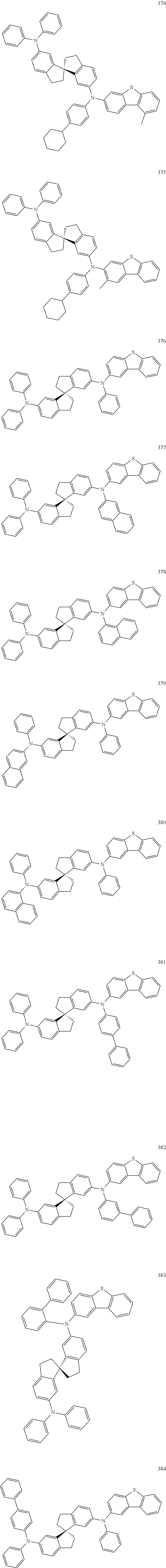

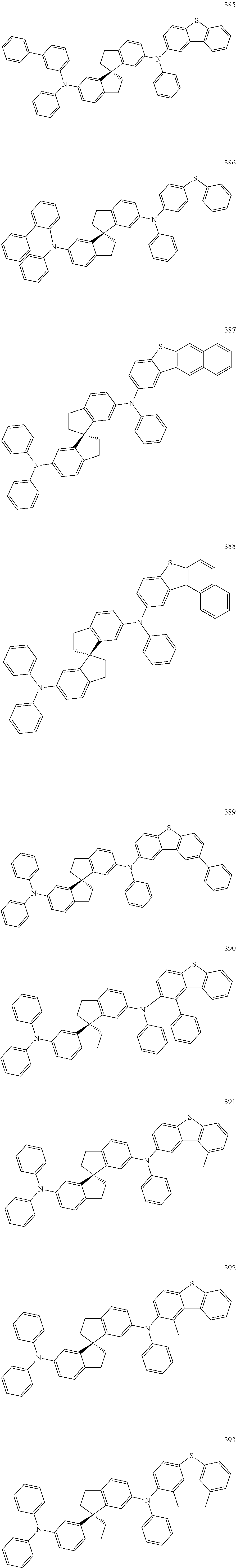

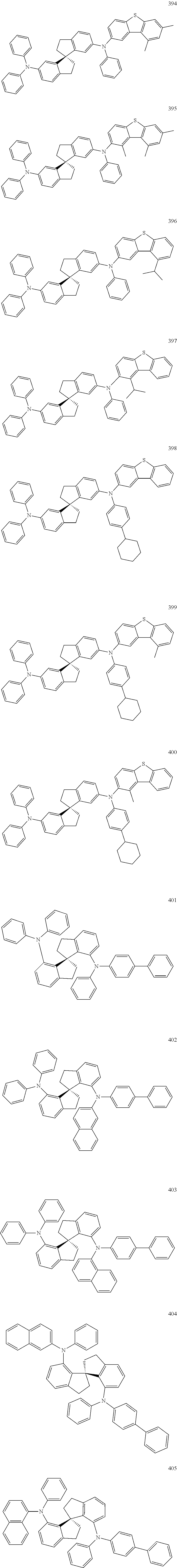

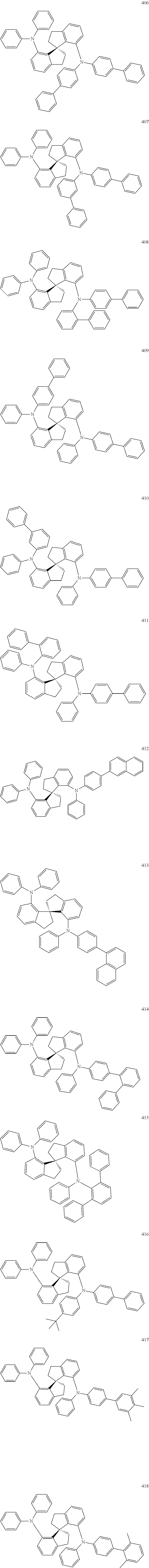

[chemical structure]

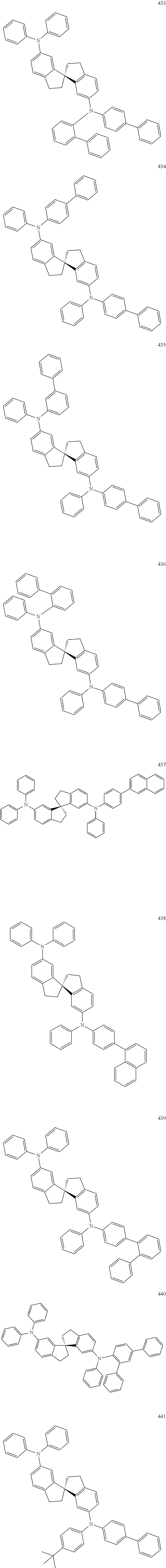

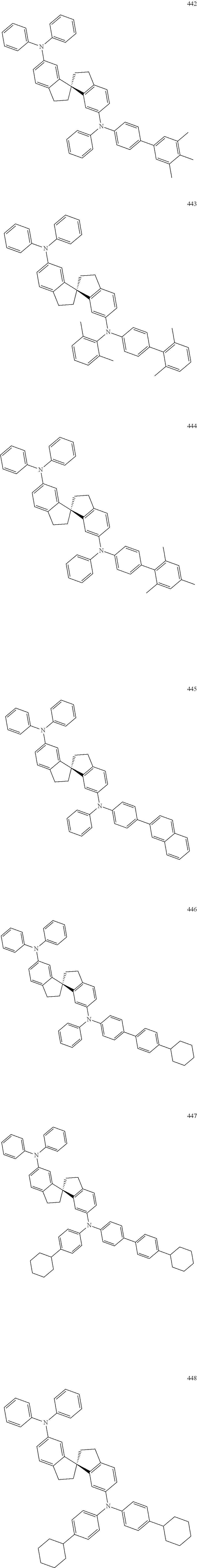

[chemical structure]

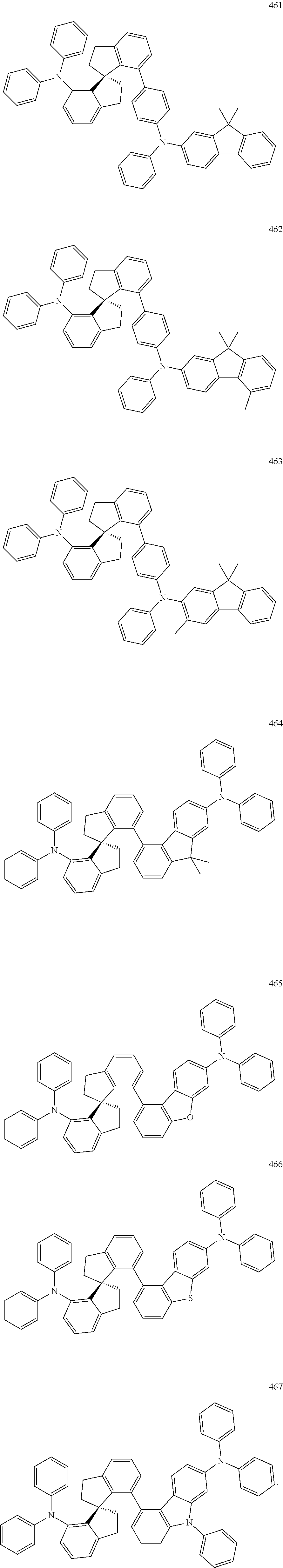

23. A display device comprising:
a base layer;
a circuit layer disposed on the base layer; and
a display element layer disposed on the circuit layer and including a light emitting element, wherein
the light-emitting element includes a first electrode, a second electrode disposed on the first electrode, and at least one functional layer between the first electrode and the second electrode, and comprising an amine compound represented by Formula 1: 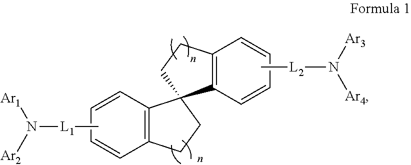
in Formula 1,
Ar₁ to Ar₄ are each independently a substituted or unsubstituted hydrocarbon ring of 6 to 30 ring-forming carbon atoms, or a substituted or unsubstituted heteroaryl group of 2 to 30 ring-forming carbon atoms,
L₁ and L₂ are each independently a direct linkage, a substituted or unsubstituted arylene group of 6 to 30 ring-forming carbon atoms, or a substituted or unsubstituted heteroarylene group of 2 to 30 ring-forming carbon atoms, and
“n” is an integer of 1 to 3.

24. The display device of claim 23, wherein
the light emitting element is a blue light emitting element.

25. The display device of claim 23, further comprising a light control layer including a quantum dot.
